(19) United States
(12) Patent Application Publication
(10) Pub. No.: US 2023/0065740 A1
(43) Pub. Date: Mar. 2, 2023

(54) CYCLIN-DEPENDENT KINASE INHIBITORS

(71) Applicant: SPV THERAPEUTICS INC., Research Triangle Park, NC (US)

(72) Inventors: Subba R. KATAMREDDY, Research Triangle Park, NC (US); Yousry SAYED, Research Triangle Park, NC (US); Naresh RAMIREDDY, Research Triangle Park, NC (US)

(21) Appl. No.: 17/416,936

(22) PCT Filed: Dec. 27, 2019
(86) PCT No.: PCT/US2019/068774

§ 371 Date: Jun. 21, 2021

Related U.S. Application Data
(60) Provisional application No. 62/785,854, filed on Dec. 28, 2018.

(51) Int. Cl. C07D 471/20; C07D 519/00

(52) U.S. Cl. CPC C07D 471/20; C07D 519/00

(57) ABSTRACT

Described herein are compounds and their pharmaceutically acceptable salts, pharmaceutical compositions thereof, methods of treatment, and medical uses. The compounds described herein are modulators of cyclin-dependent kinases, and are useful in the treatment or alleviation of protein kinase associated disorders, including cancer, infectious diseases, autoimmune diseases, or cardiovascular diseases.

### CROSS-REFERENCE TO RELATED APPLICATIONS

The present application claims the benefit of U.S. Provisional Patent Application No. 62/785,854, filed Dec. 28, 2018, which is hereby incorporated herein by reference in its entirety.

### TECHNICAL FIELD

Described herein are compounds and their pharmaceutically acceptable salts, pharmaceutical compositions thereof, methods of treatment, and medical uses. The compounds described herein are modulators of cyclin-dependent kinases and are useful in the treatment or alleviation of protein kinase associated disorders, including cancer, infectious diseases, autoimmune diseases, or cardiovascular diseases.

### BACKGROUND

The cell cycle is a complex series of events that cause a cell to divide and duplicate. The phases of the cell cycle include mitosis (M), gap 1 phase (G1), synthesis phase (S) and gap 2 phase (G2). Checkpoints in the cell cycle ensure that division only occurs after sufficient growth and faithful DNA replication and under favorable conditions. At each checkpoint, a variety of proteins engage in a series of carefully coordinated biochemical reactions. This complexity allows for precise regulation of all steps in the cell cycle.

Cyclin-dependent kinases (CDKs) are powerful protein kinase enzymes that drive cell cycle regulation in biochemical pathways by modulating the activity of various target proteins of the cell cycle. In response to a variety either intra- or extracellular cues, the enzymes function by the transfer of phosphate groups from intracellular adenosine triphosphate (ATP) to the serine or threonine amino acids of target proteins involved in a signaling pathway. These activating or inhibitory phosphorylation events act as the signals that either activate or deactivate target protein activity, and thus stimulate or inhibit the cell from entering the cell cycle and dividing.

CDKs themselves require the presence of cyclin proteins to activate. A variety of cyclin proteins exist, each of which is present at varying protein levels and act during a specific stage of the cell cycle by binding to specific CDKs. The cyclin-CDK complexes then recognize and modify multiple target substrates and thus coordinate multiple events during each phase of the cell cycle. Thus, the various activities of the cell cycle are determined by the specific combination of cyclins and CDKs that are active at each stage. For example, cyclin D and CDK4 and CDK6 have been found to be active during the G1 phase of the cell cycle and react to external signals such as growth factors and mitogens. Cyclins A and E and CDK2 have been found to be active during G1/S phase of the cell cycle and are thought to regulate centrosome duplication. Some cyclin-CDK complexes are found to be involved in transcription activities, such as cyclin A, cyclin E and CDK2 which are thought to target helicase and polymerase protein enzymes. CDK-cyclin controlled protein phosphorylation thus plays an important role in many key cellular processes including cell division, metabolism, survival, and apoptosis.

The ubiquitous nature of CDKs coupled with the highly regulated and complex nature of CDK dependent phosphorylation makes it susceptible to malfunction. Thus, CDKs are implicated in a variety of cell-cycle dependent diseases, including cancers, cardiovascular diseases, neurodegenerative disorders, autoimmune diseases and infectious diseases with the CDKs either the causative agents or as therapeutic targets.

Of particular interest are CDK4, CDK6, and CDK9 as therapeutic targets. These cyclin-dependent kinases are involved in cell cycle regulation and have been found to be deregulated and overactive in cancer cells as result of overexpression of relevant genes or loss of naturally occurring, endogenous inhibitors by gene deletion or mutation. Thus, inhibitors of CDK4/6/9 among other cyclin dependent kinases are important targets for cancer therapeutics, alone or in combination with other drugs.

Thus, there remains a need for additional cyclin dependent kinase inhibitors suitable for pharmaceuticals.

### SUMMARY

One embodiment described herein is a compound or a pharmaceutically acceptable salt thereof comprising Formula I or II:

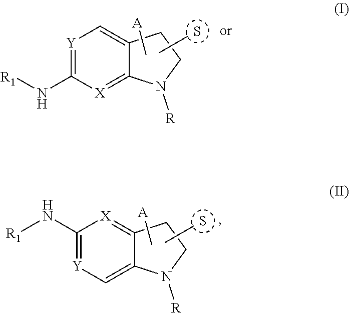

wherein
A is none, Me, CO, or gem-difluoro;
X and Y are independently, C, N, or C—F; [symbol]
is

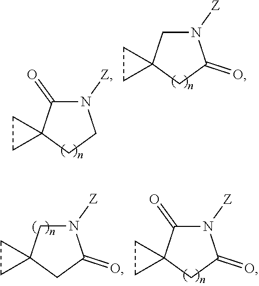

bonded in a spiro-manner,

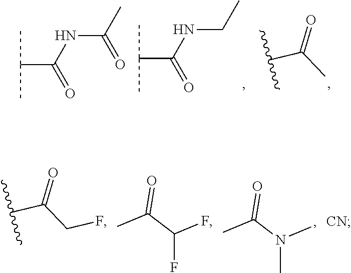

where Z is H, Me, or CH₂CH₂OH, CH₂CH₂CN, CH₂CH₂F, COCH₃, COCH═CH; and n is 0, 1, 2, or 3;
R is (CH₂)ₙ,

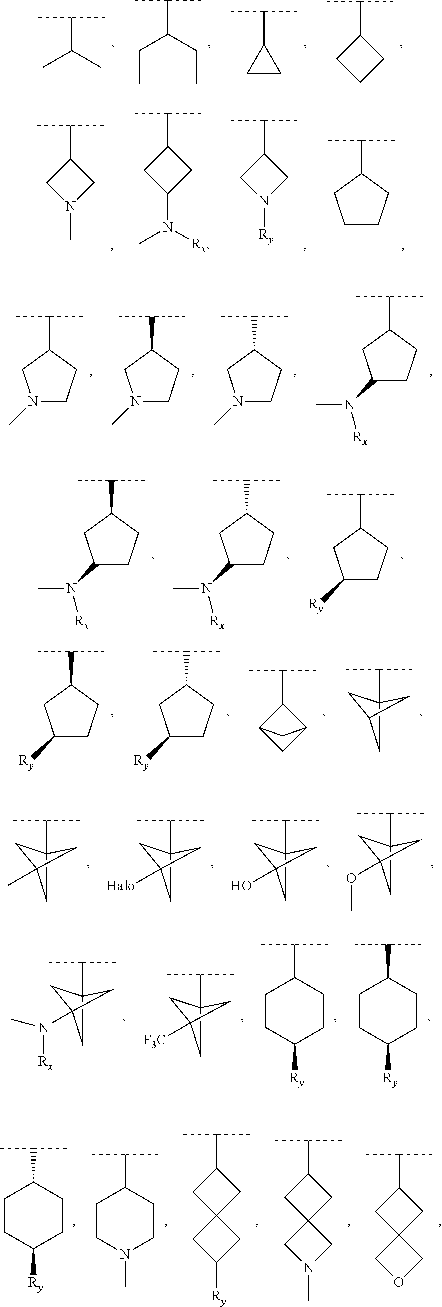

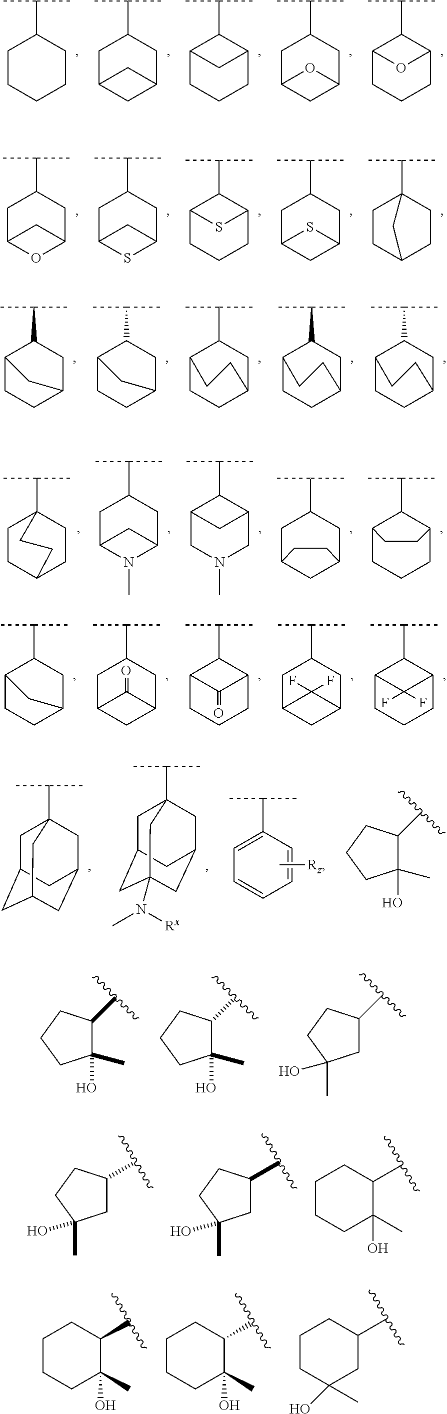

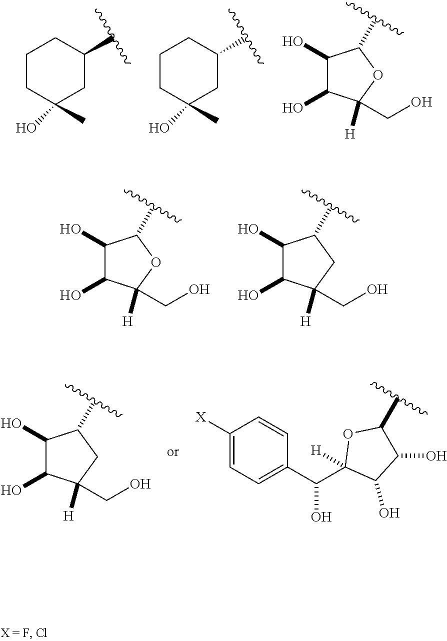

where Rₓ is H or Me, R_{y} is Me, OH, or OMe; R_{z} is H, Me, OMe, OH, N, F, or CF₃; and n is 1 to 11; and

### R₁ is:

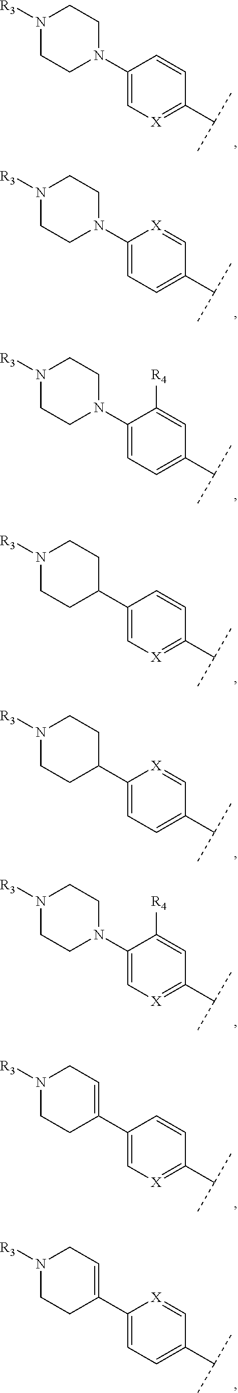

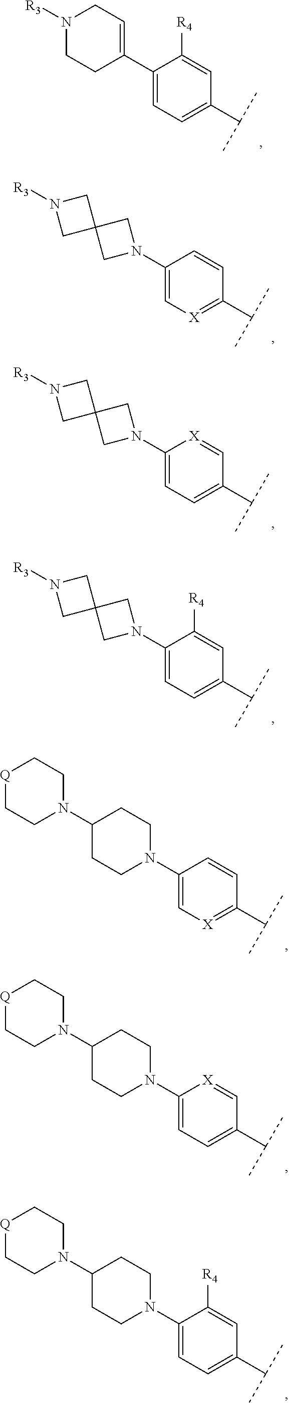

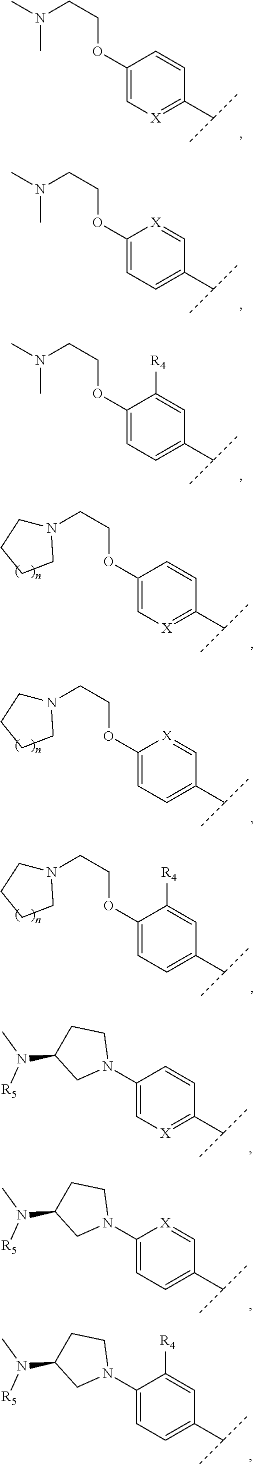

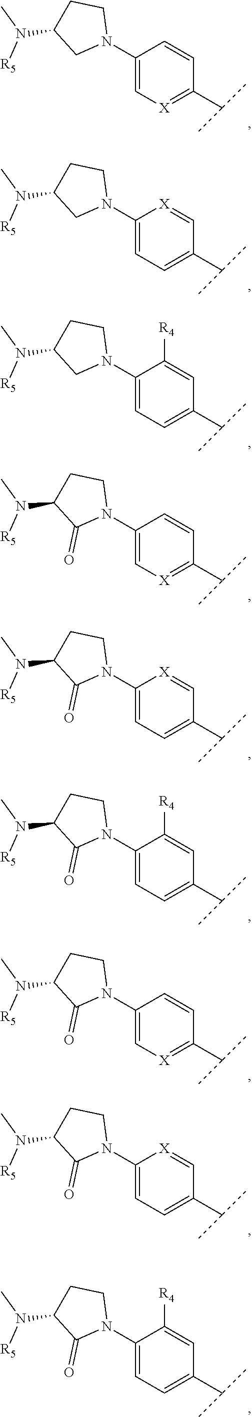

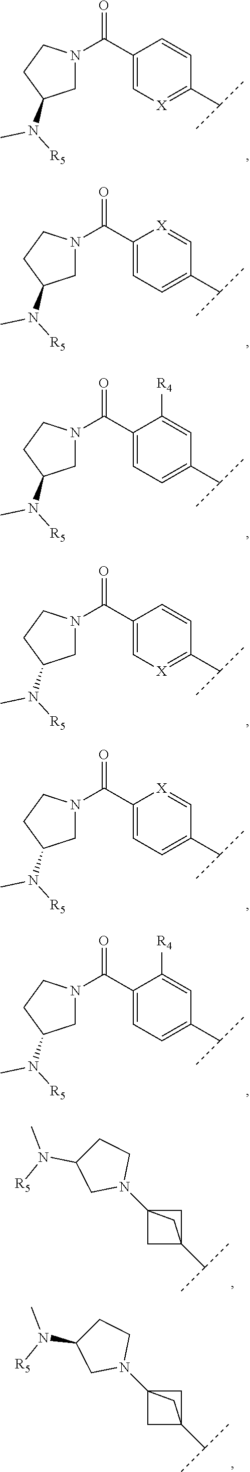

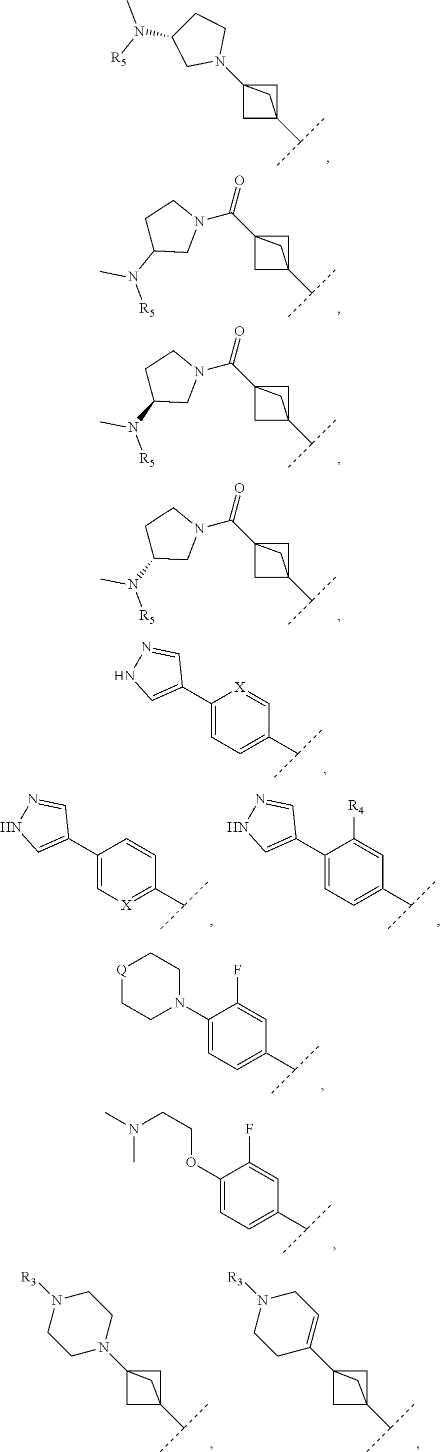

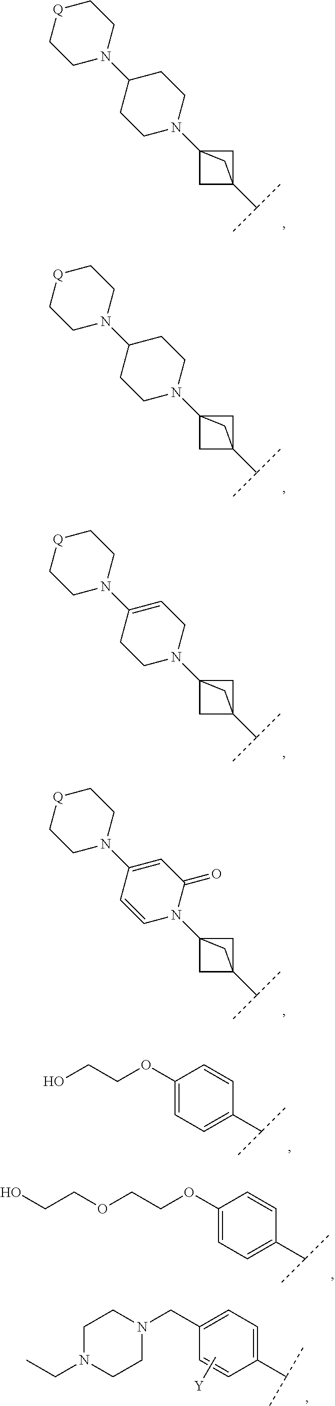

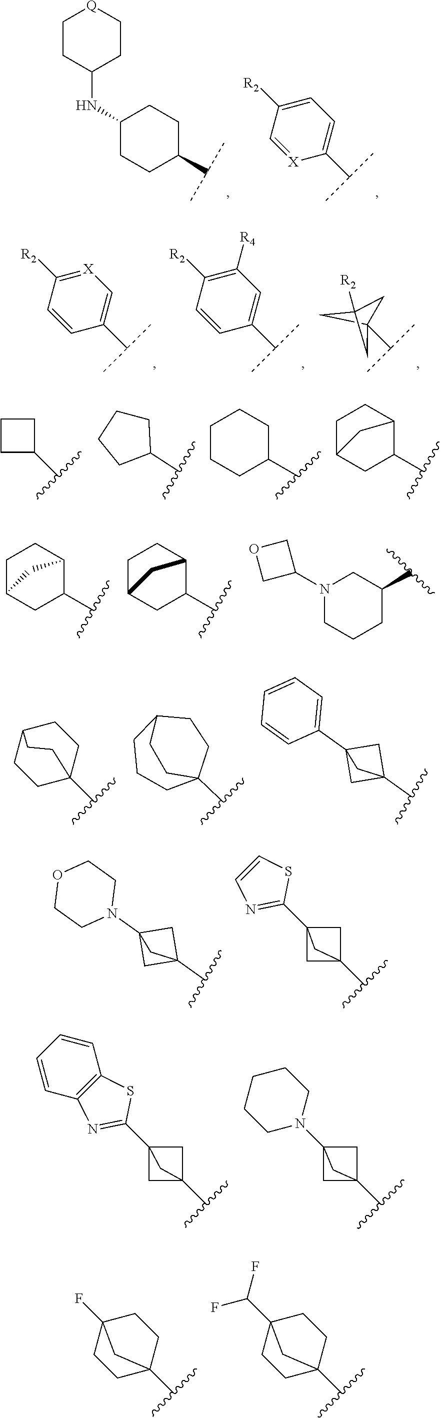

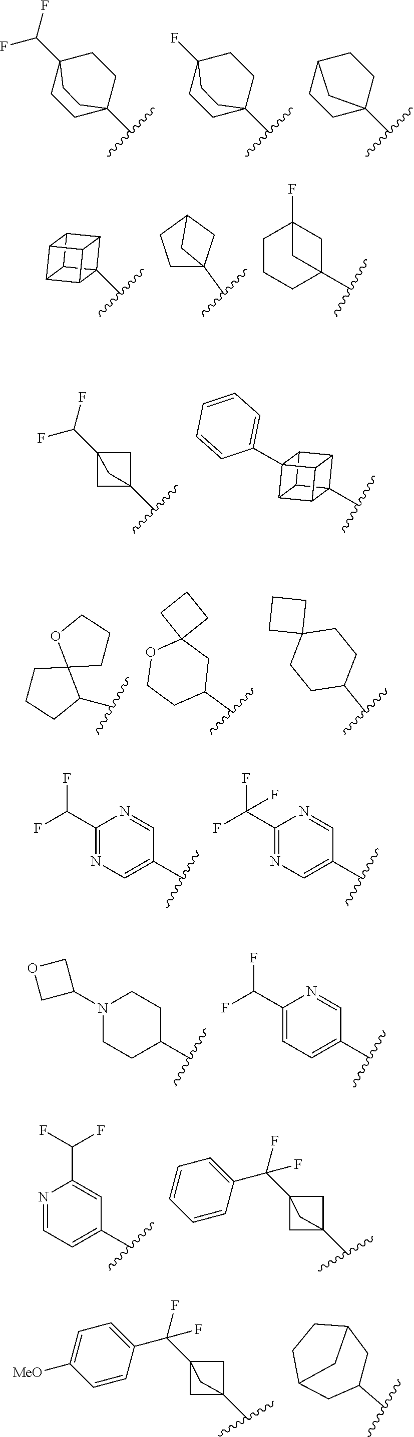

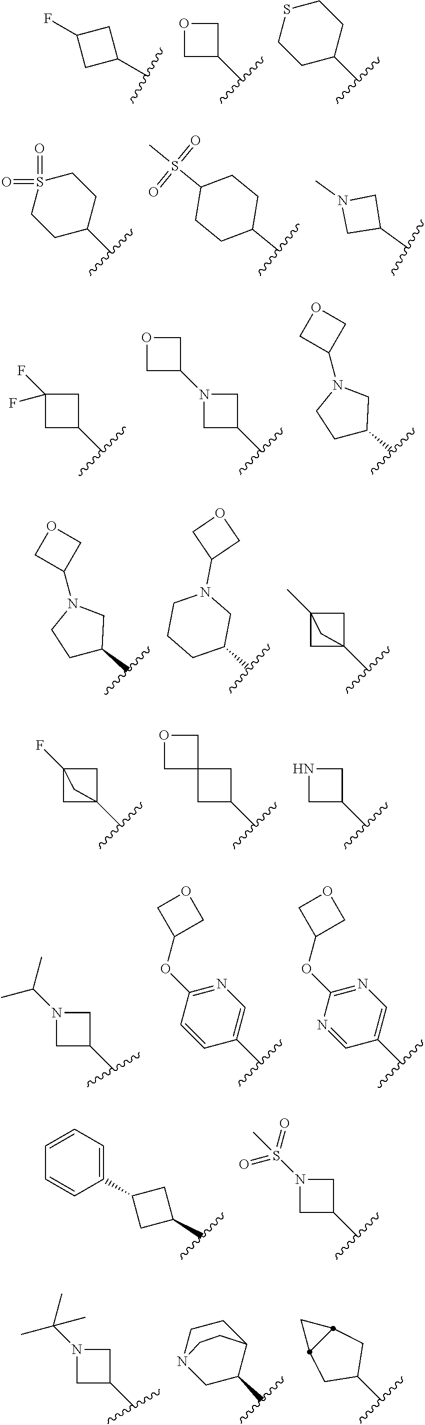

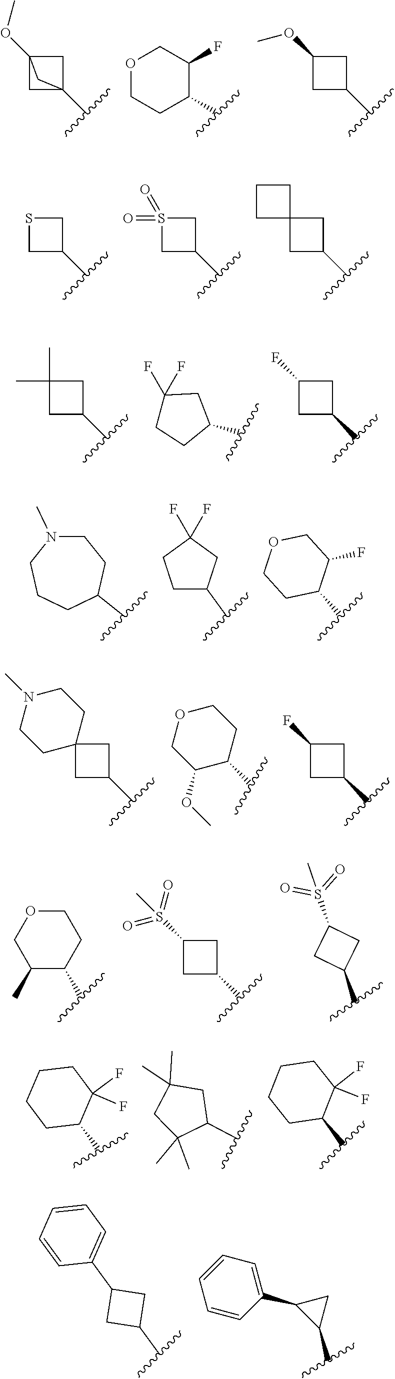

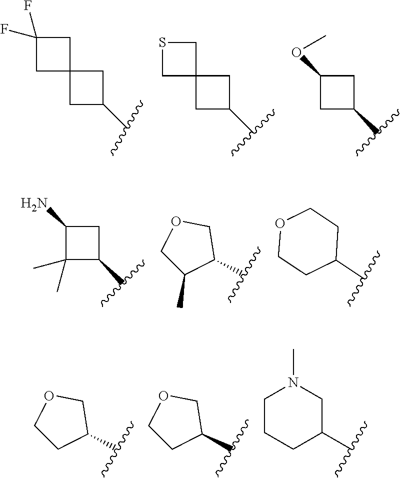

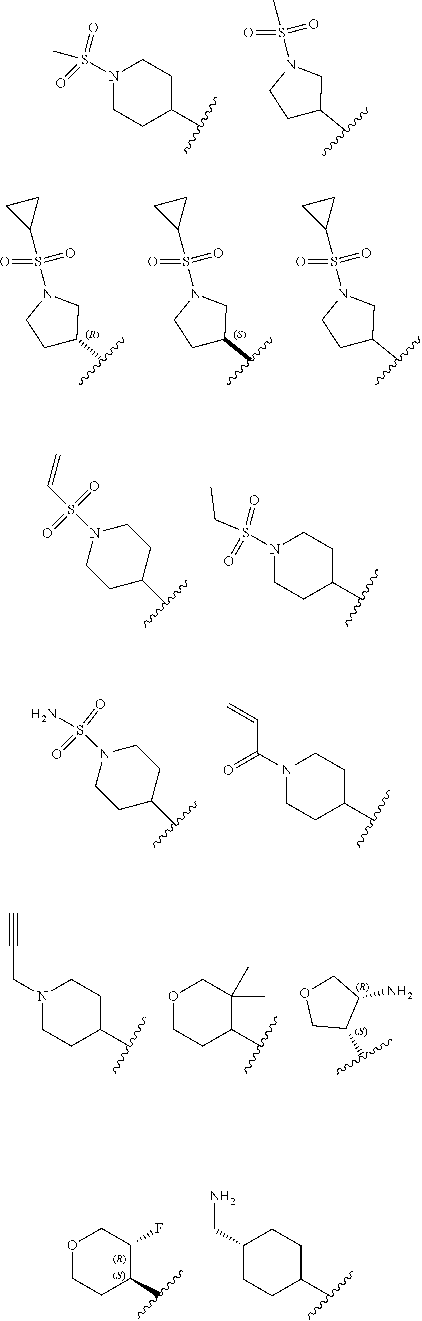

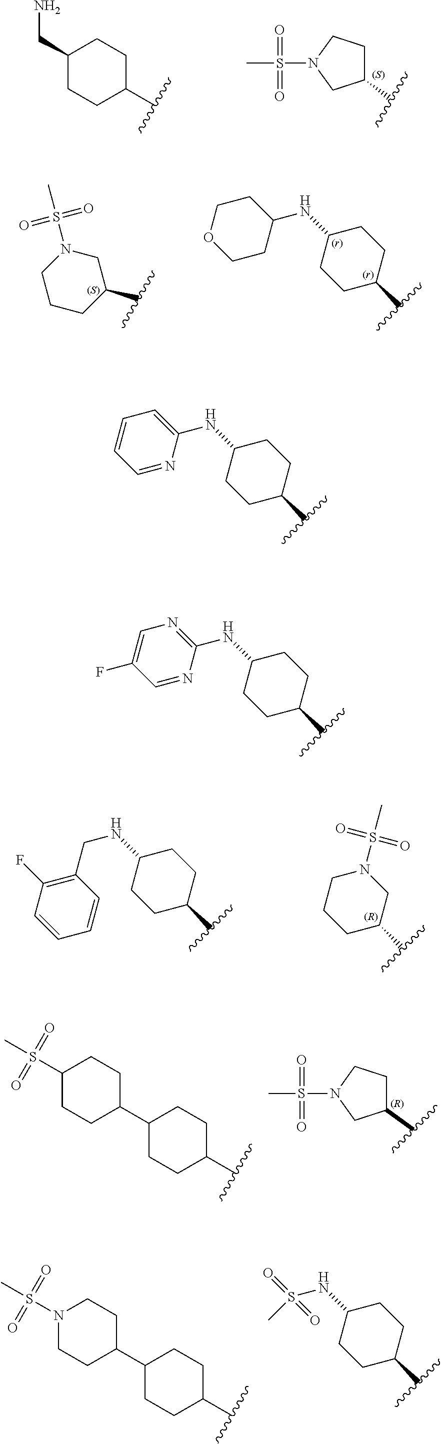

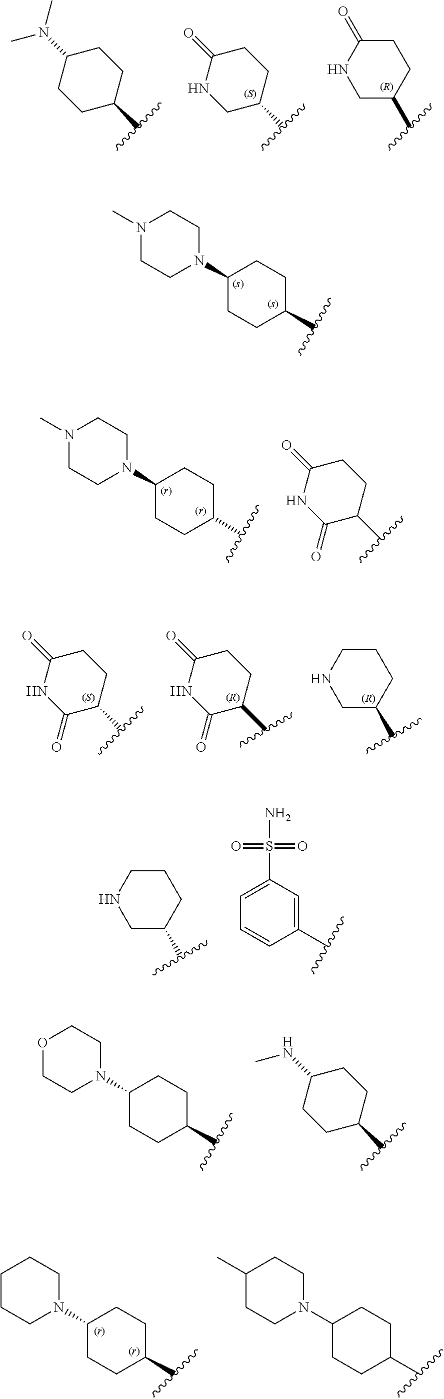

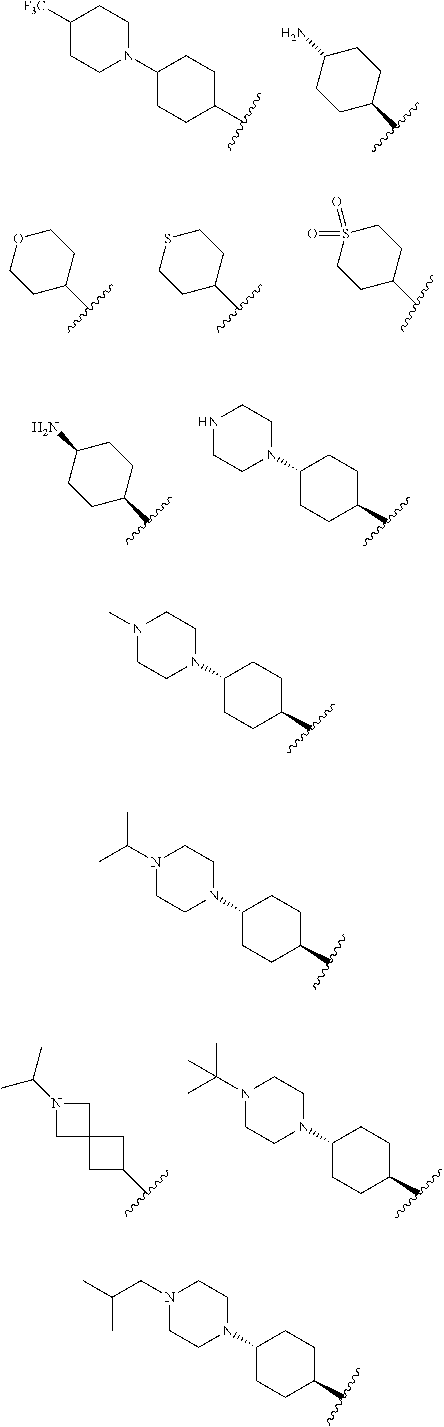

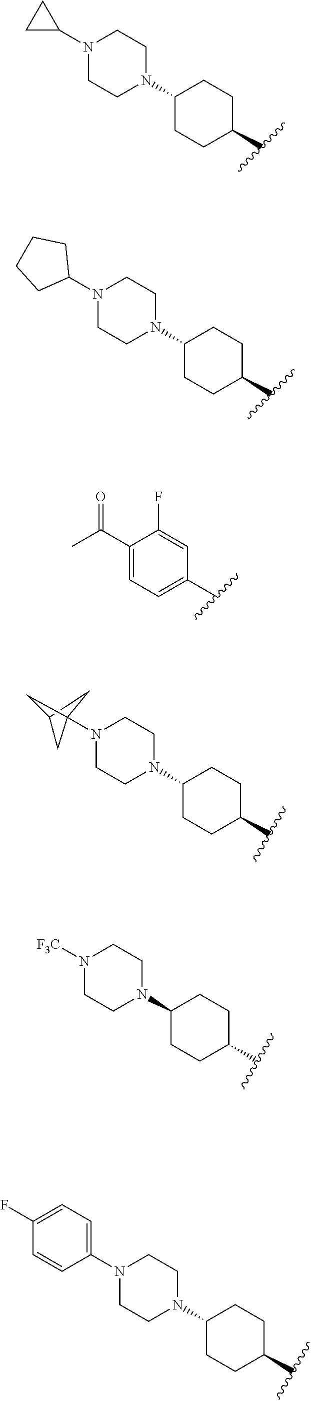

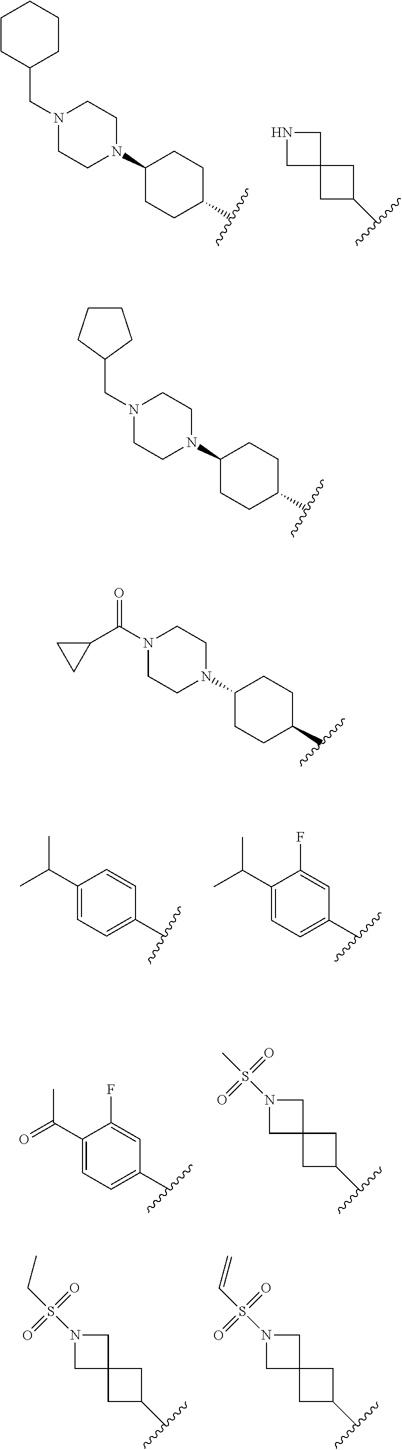

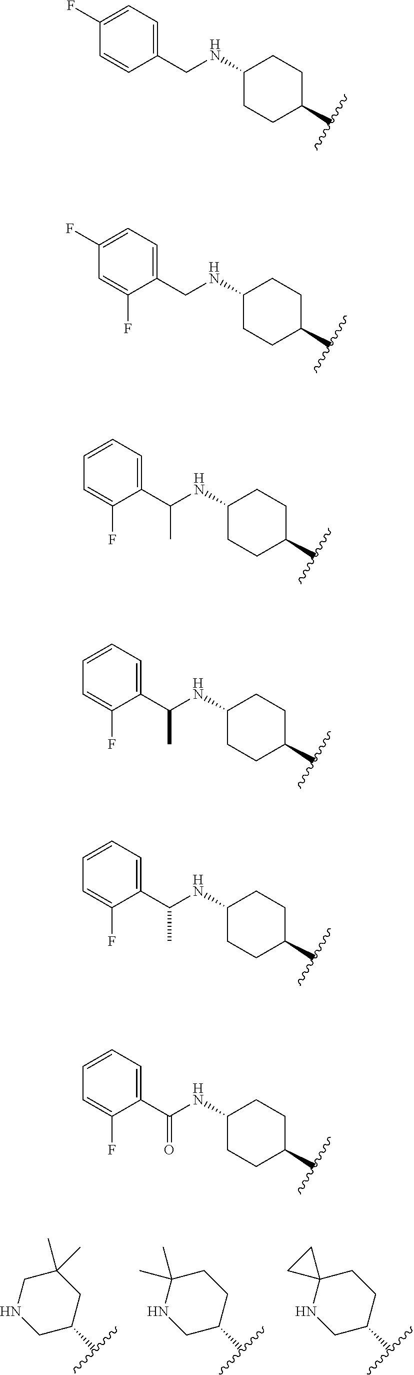

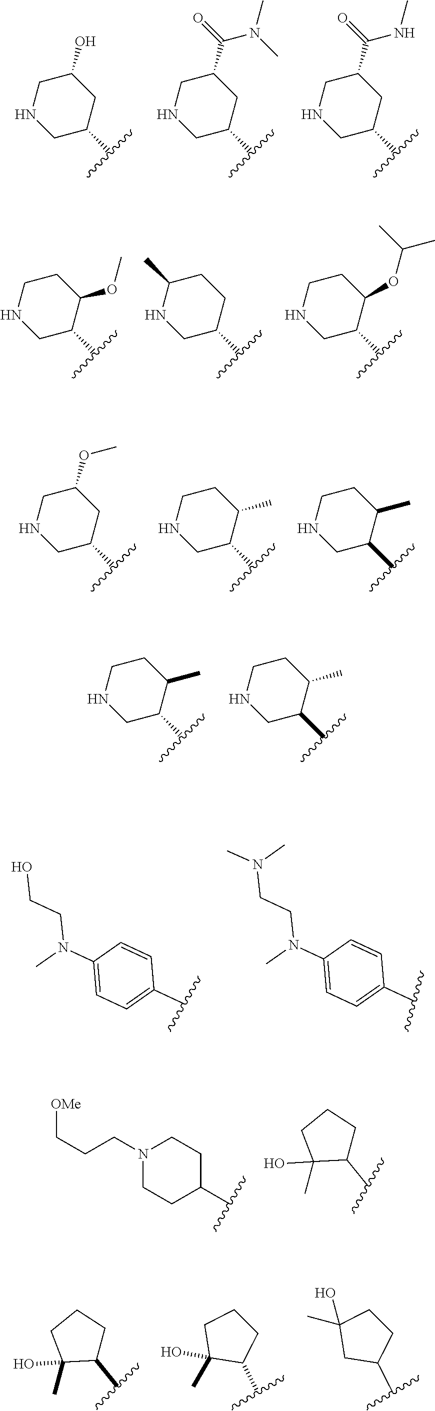

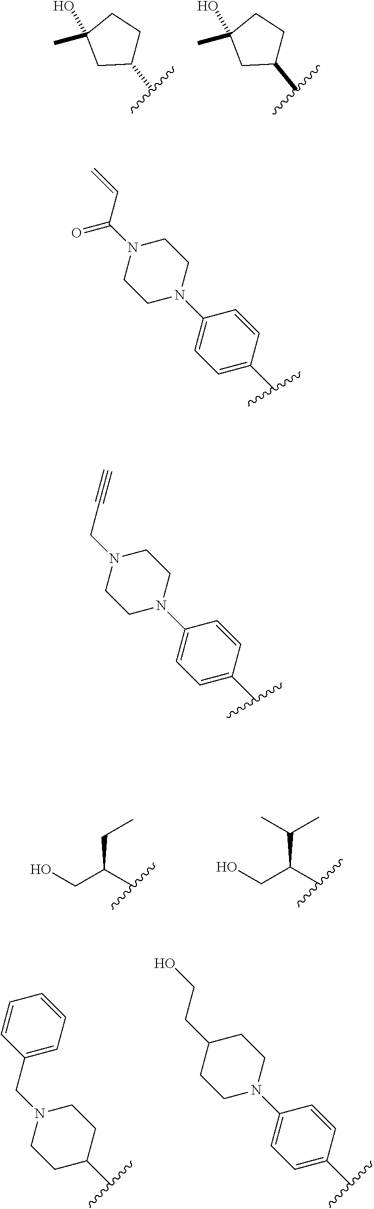

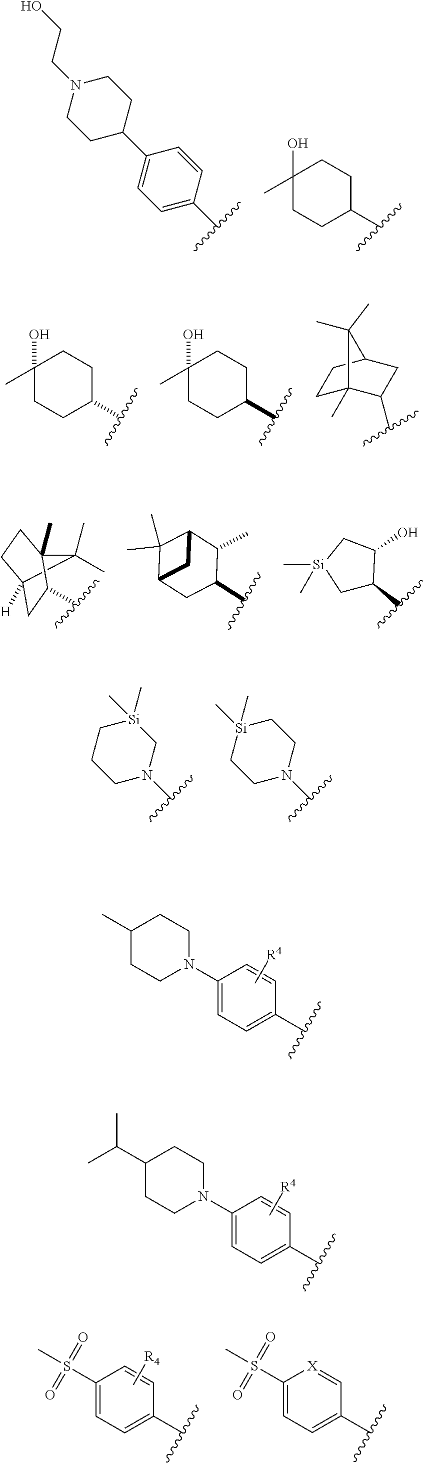

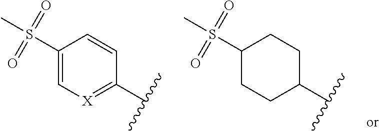

where R₃ is H, Me, CH₂CH₂OH, isopropyl, isobutyl, or bicyclo[1.1.1]pentane;
R₄ is H, Me, OMe, CF₃, or CHF₂;

### R₅ is H or Me;

### X is C—H, N, or C—F;

Y is H, N, F, Me, OMe, CF₃, or CHF₂;

### Q is O or S;

n is 1, 2, or 3; and

### R₂ is:

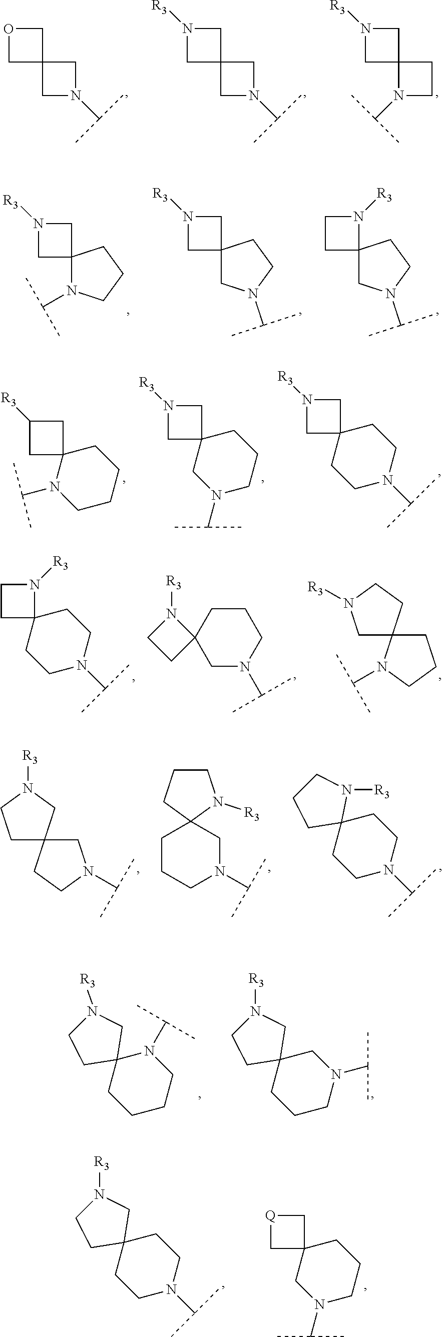

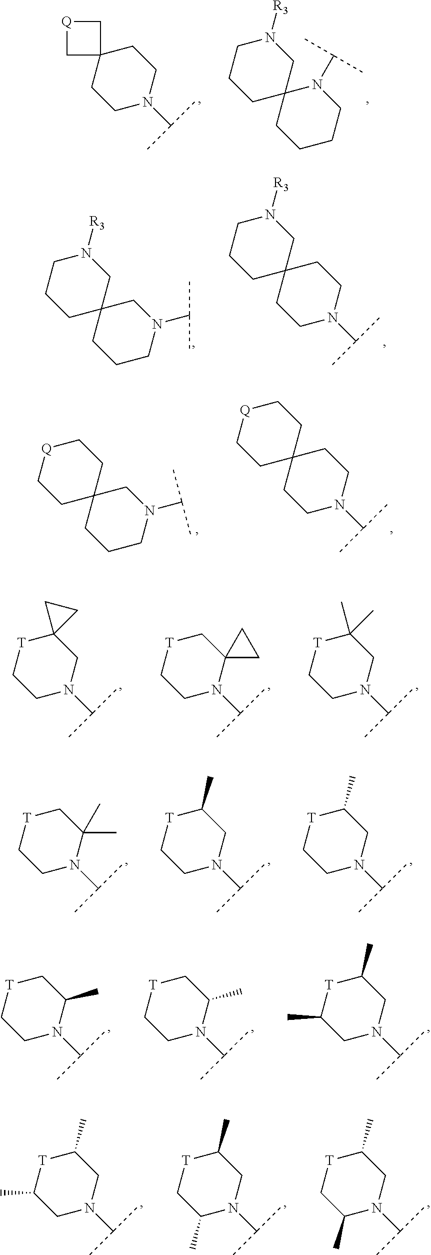

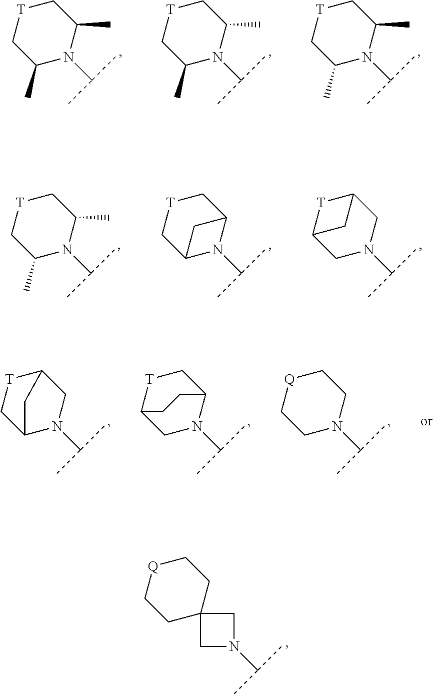

where T is O, NH, NMe, N-isopropyl, or N-isobutyl. In another aspect, [symbol] is

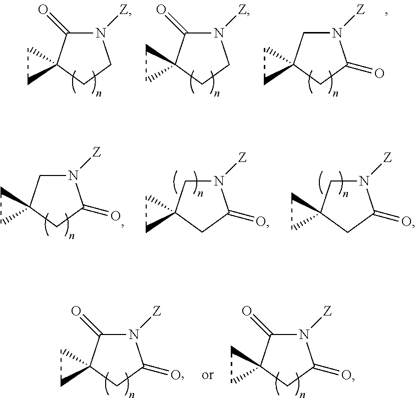

bonded in a spiro-manner,

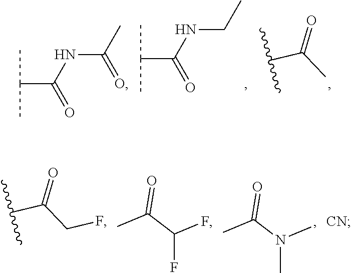

where Z is H, Me, or CH₂CH₂OH, CH₂CH₂CN, CH₂CH₂F, COCH₃, COCH═CH; and n is 0, 1, 2, or
3. In one aspect, R is

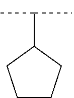

In another aspect, R₁ is:

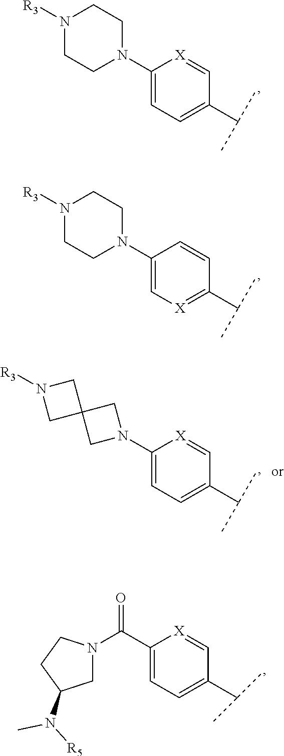

where R₃ is H, Me, isopropyl, isobutyl, CH₂CH₂OH, or bicyclo[1.1.1]pentane; R₅ is H or Me; and X is C—H, N, or C—F. In another aspect, the compound comprises Formula I:

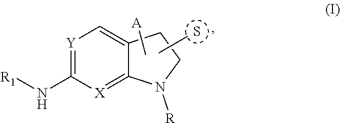

wherein A is none; and X and Y are independently, C or N. In another aspect, [symbol] is

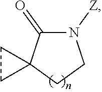

bonded in a spiro-manner; where Z is H, Me, or CH₂CH₂OH; and n is 0, 1, 2, or 3. In another aspect, R is

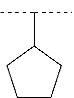

In another aspect, R₁ is:

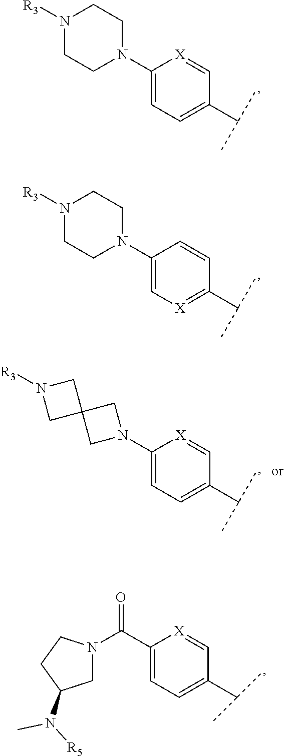

where R₃ is H, Me, isopropyl, isobutyl, CH₂CH₂OH, or bicyclo[1.1.1]pentane; R₅ is H or Me; and X is C—H, N, or C—F.

Another embodiment described herein is a compound or a pharmaceutically acceptable salt thereof comprising Formulae III-VIII:

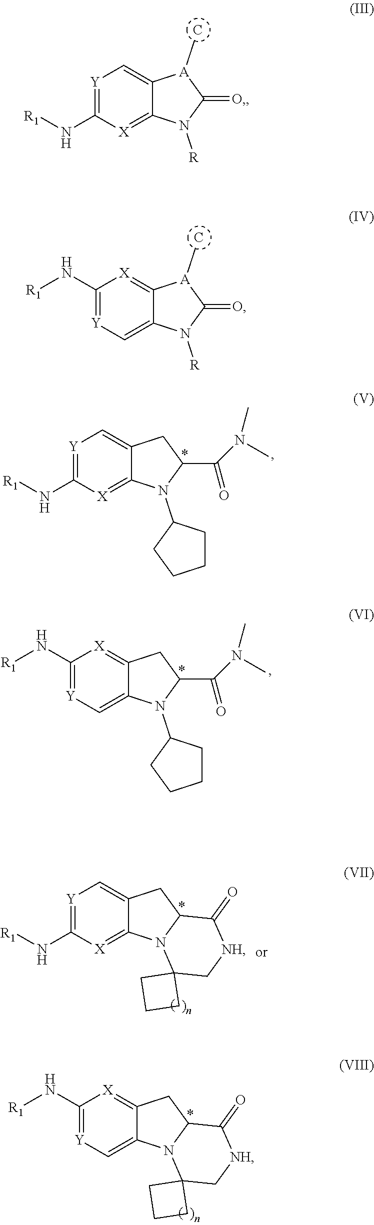

wherein: A is C or N; X and Y are independently N, C, or C—F; [symbol]
is

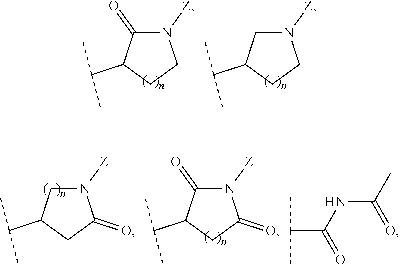

or

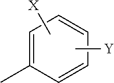

X=Y=H; X=Cl, F, Me, OMe, Y=H: X=Y=Cl, F, Me, OMe, where Z is H, Me, or CH₂CH₂OH; and n is 0, 1, 2, or 3;
R is (CH₂)ₙ,

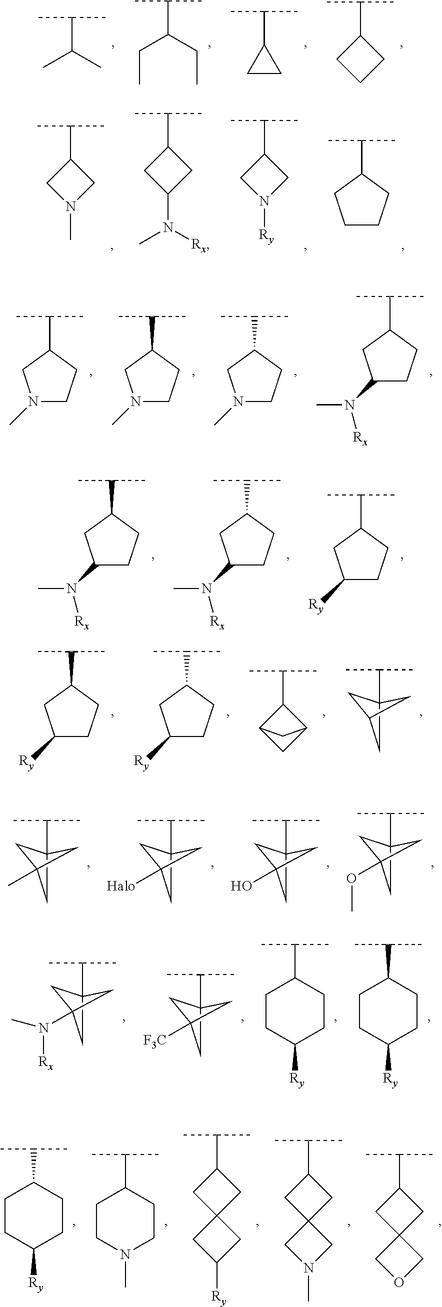

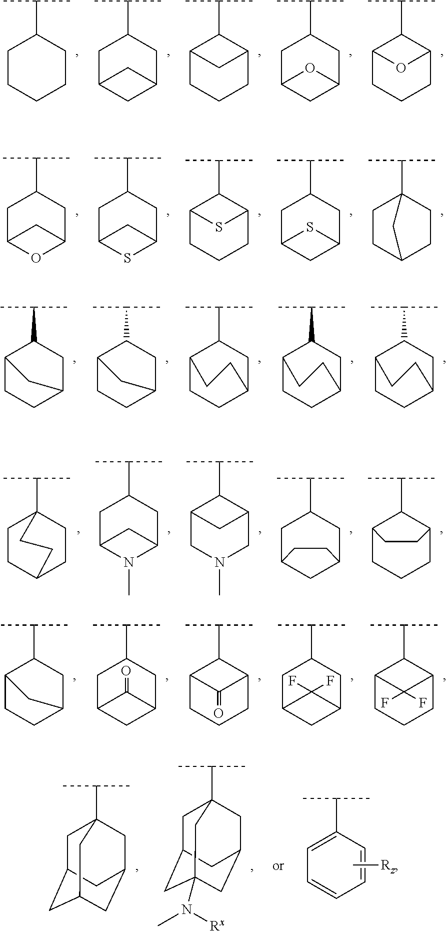

where Rₓ is H or Me, R_{y} is Me, OH, or OMe; R_{z} is H, Me, OMe, OH, N, F, or CF₃; and n is 1 to 11; and

### R₁ is:

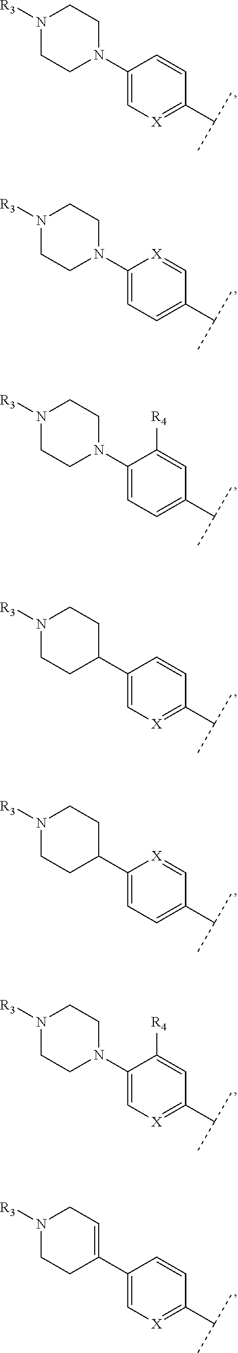

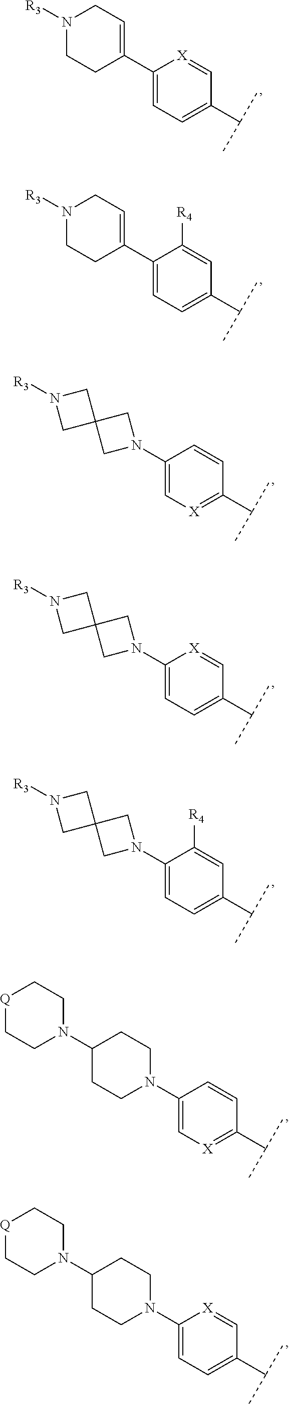

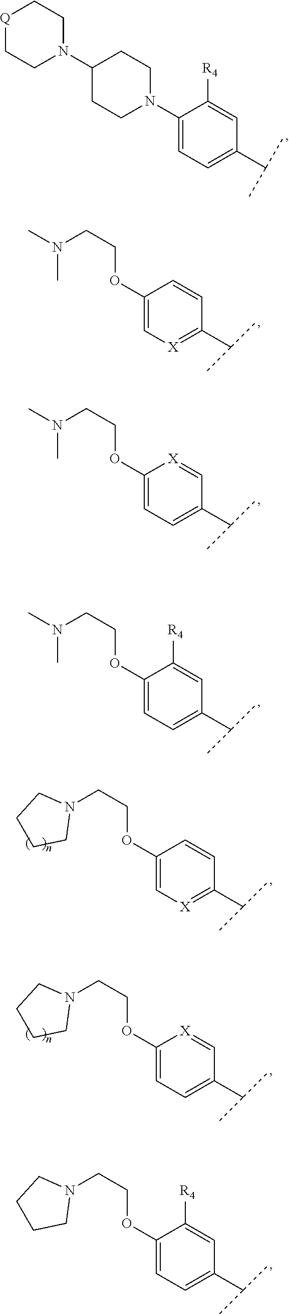

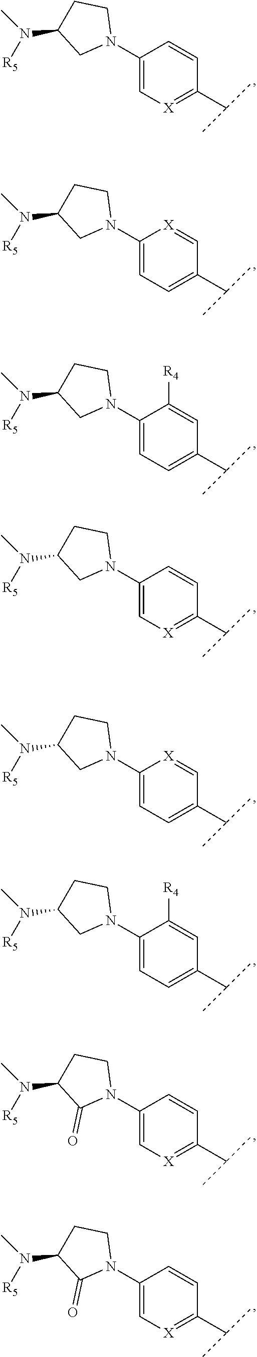

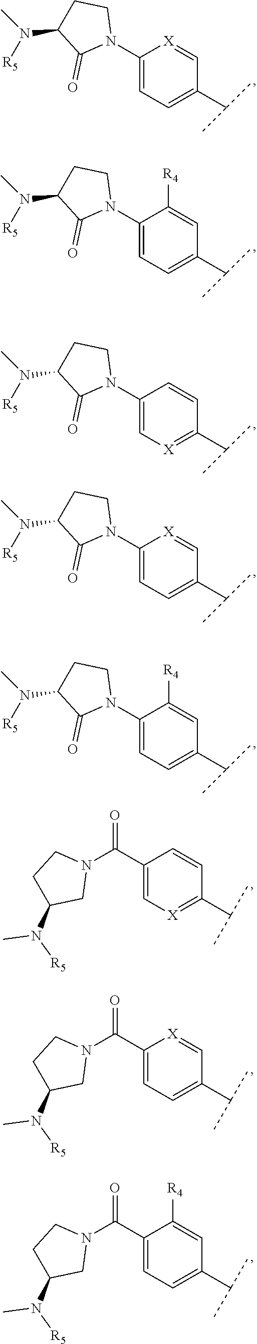

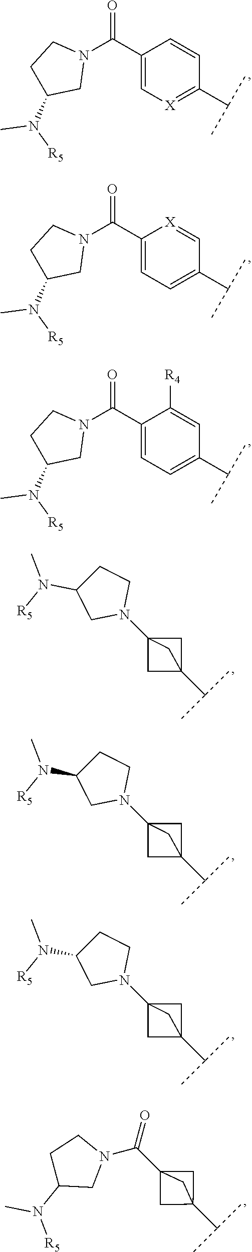

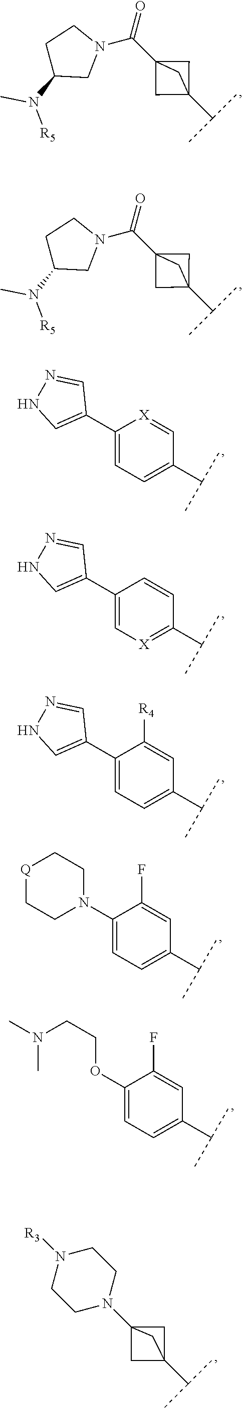

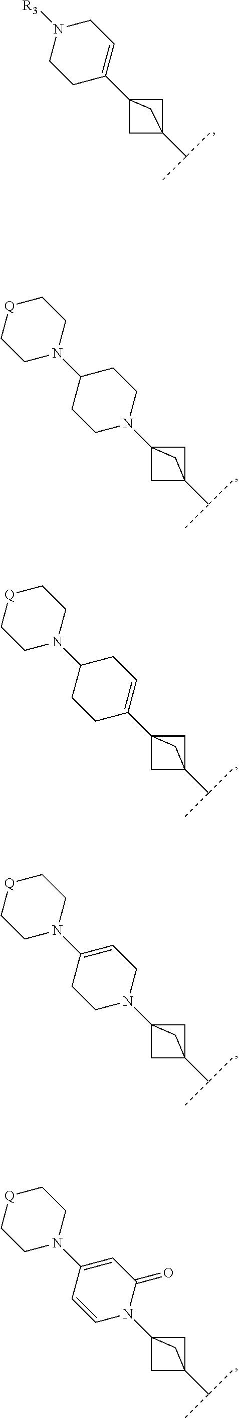

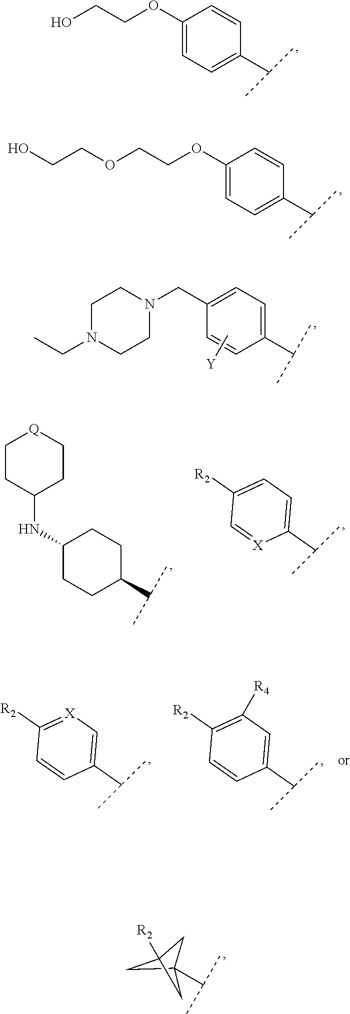

where R₃ is H, Me, isopropyl, isobutyl, CH₂CH₂OH, or bicyclo[1.1.1]pentane;
R₄ is H, Me, OMe, CF₃, or CHF₂;

### R₅ is H or Me;

### X is C—H, N, or C—F;

Y is H, N, F, Me, OMe, CF₃, or CHF₂;

### Q is O or S;

n is 1, 2, or 3; and

### R₂ is:

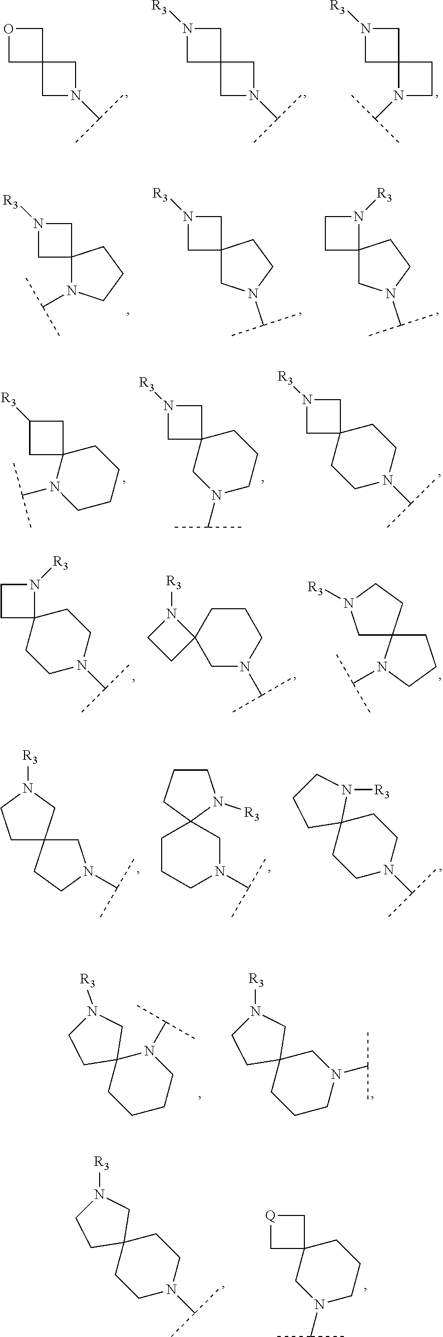

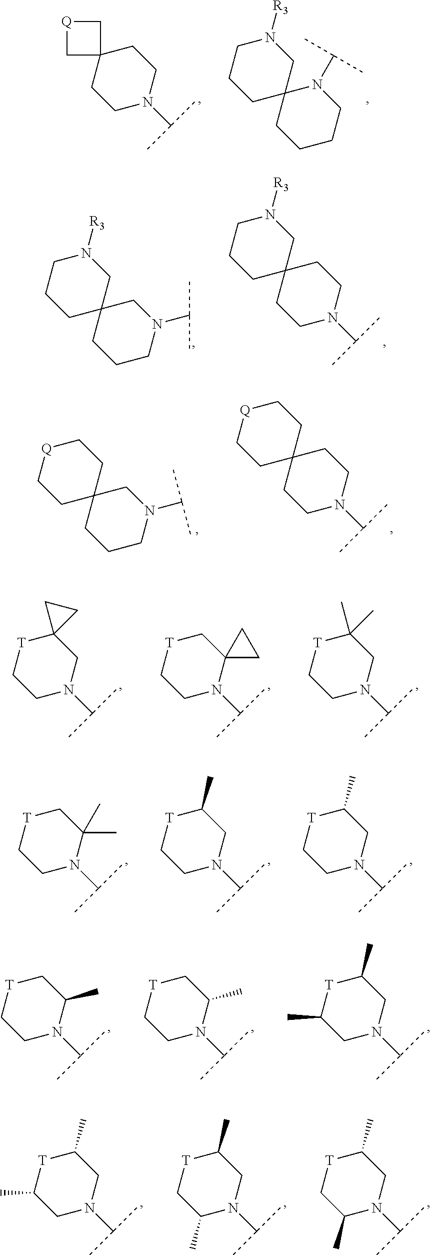

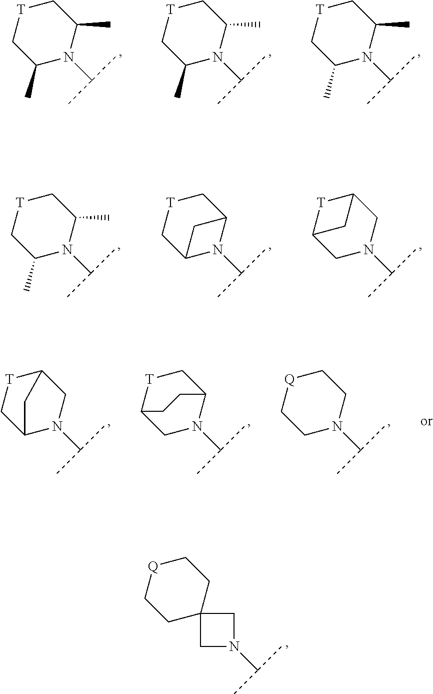

where T is O, NH, NMe, N-isopropyl, or N-isobutyl. In another aspect, compound comprises Formula (III):

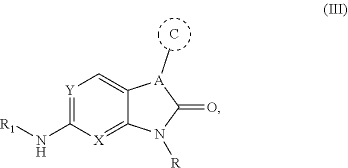

wherein: A is C or N; and X and Y are independently N, C, or C—F. In another aspect, [symbol] is:

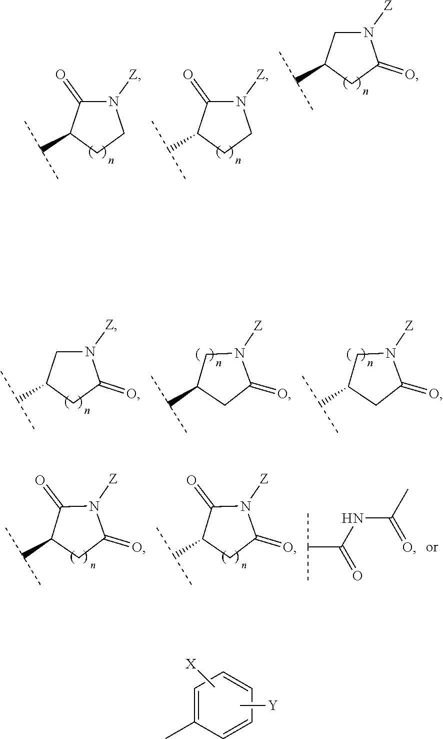

X=Y=H; X=Cl, F, Me, OMe, Y=H: X=Y=Cl, F, Me, OMe, where Z is H, Me, or CH₂CH₂OH; and n is 0, 1, 2, or 3. In another aspect, R is

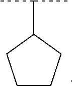

In another aspect, R₁ is:

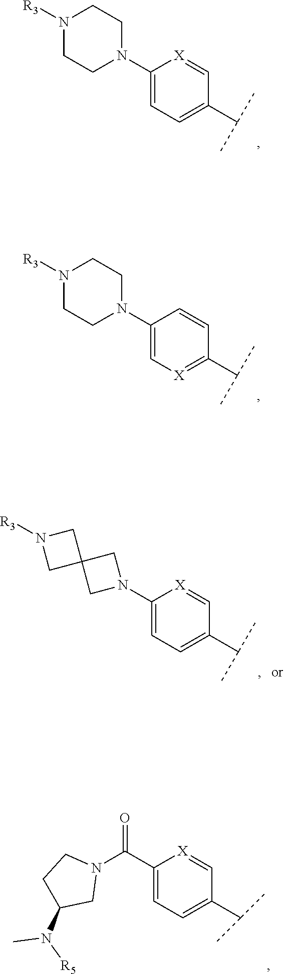

where R₃ is H, Me, isopropyl, isobutyl, CH₂CH₂OH, or bicyclo[1.1.1]pentane; R₅ is H or Me; and X is C—H, N, or C—F. In another aspect, the compound comprises Formula (III):

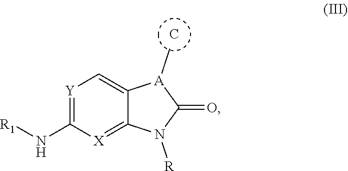

wherein: A is C;
X and Y are independently N; [symbol]
is

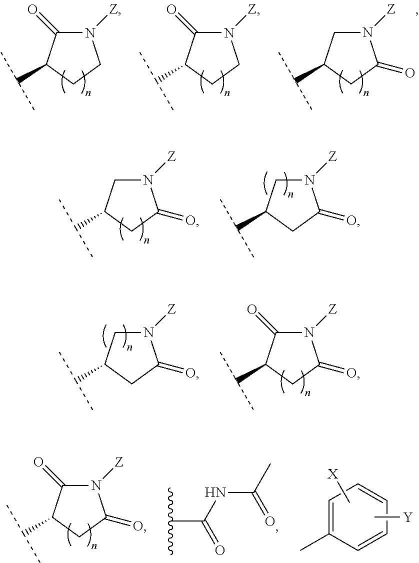

X=Y=H; X=Cl, F, Me, OMe, Y=H: X=Y=Cl, F, Me, OMe, where Z is H, Me, or CH₂CH₂OH; and n is 0, 1, 2, or 3;

### R is

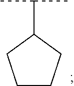

and

### R₁ is:

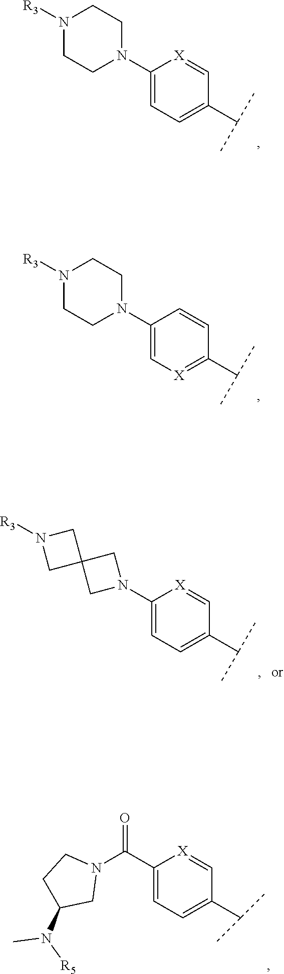

where R₃ is H, Me, isopropyl, isobutyl, CH₂CH₂OH, or bicyclo[1.1.1]pentane; R₅ is H or Me; and X is C—H, N, or C—F.

Another embodiment described herein is a compound or a pharmaceutically acceptable salt thereof comprising Formulae IX-XX:

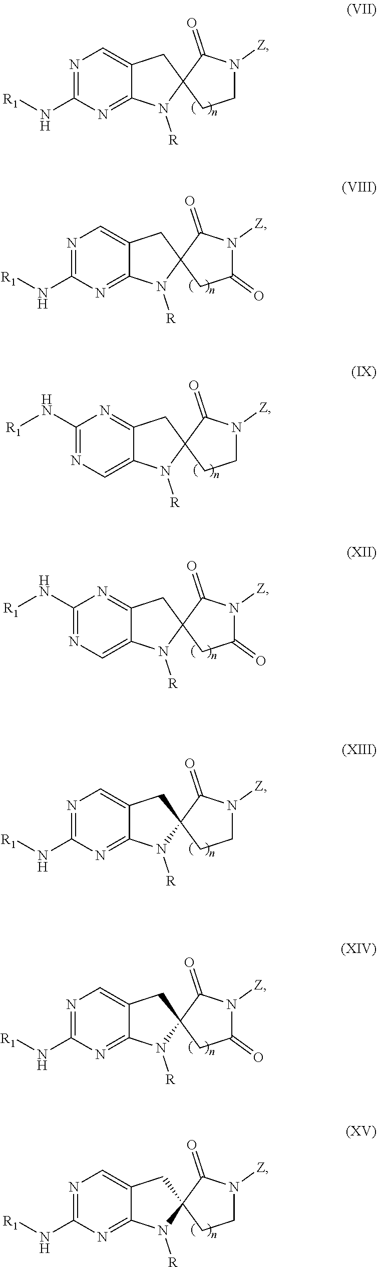

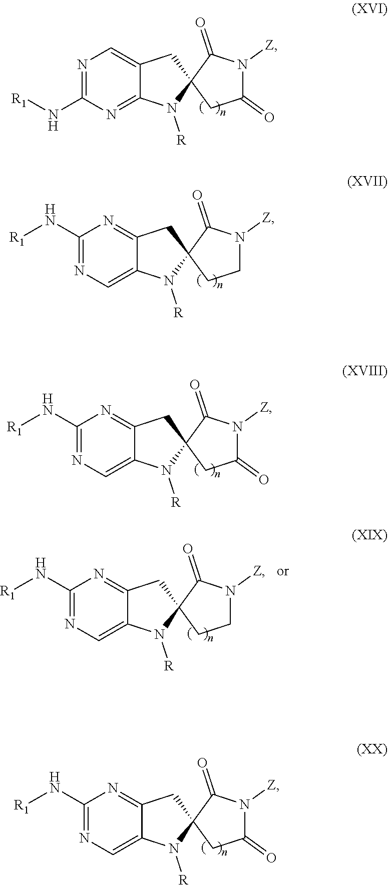

where Z is H, Me, or CH₂CH₂OH, and n is 0, 1, 2, or 3;
R is —(C₂)ₙ,

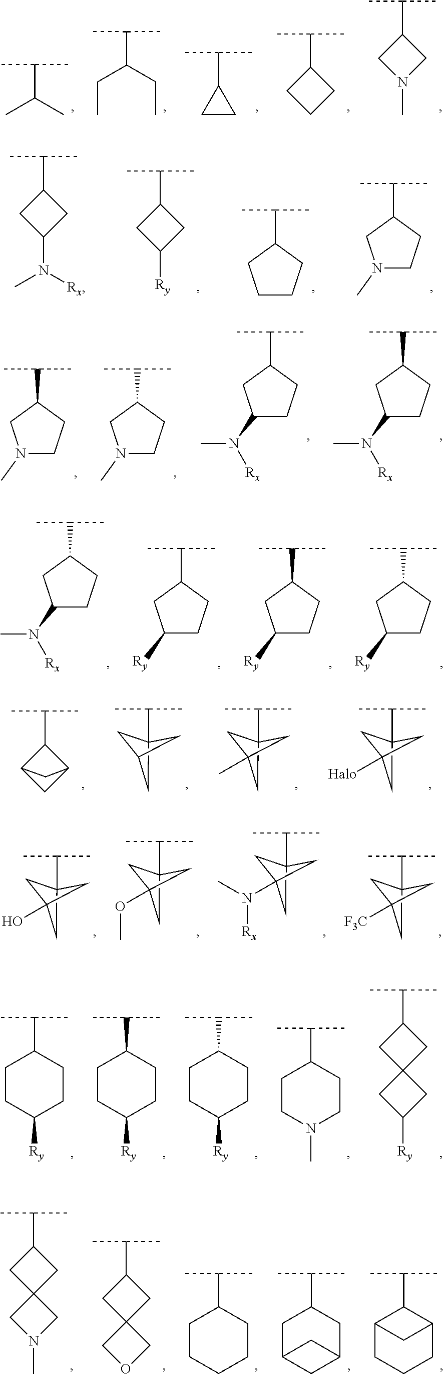

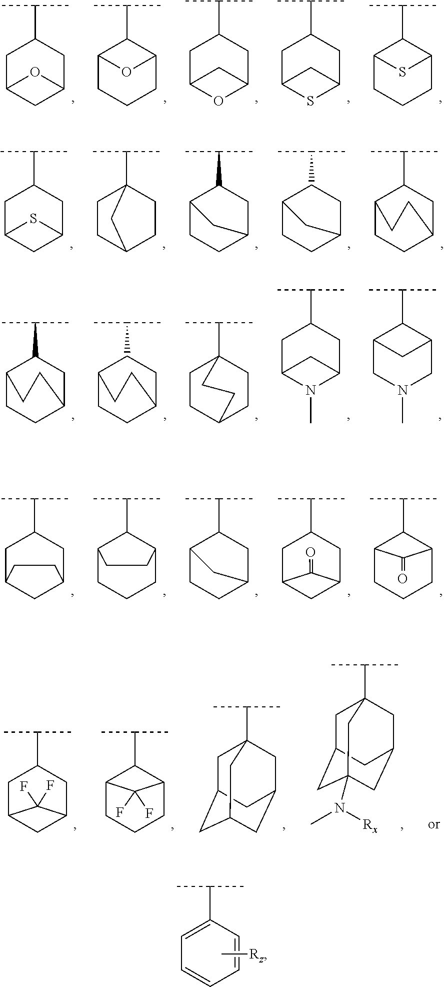

where Rₓ is H or Me, R_{y} is Me, OH, or OMe; R_{z} is H, Me, OMe, OH, N, F, or CF₃; and n is 1 to 11; and

### R₁ is:

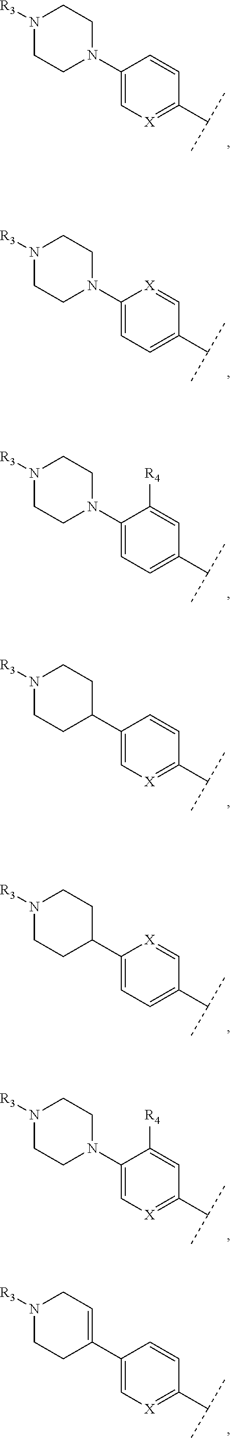

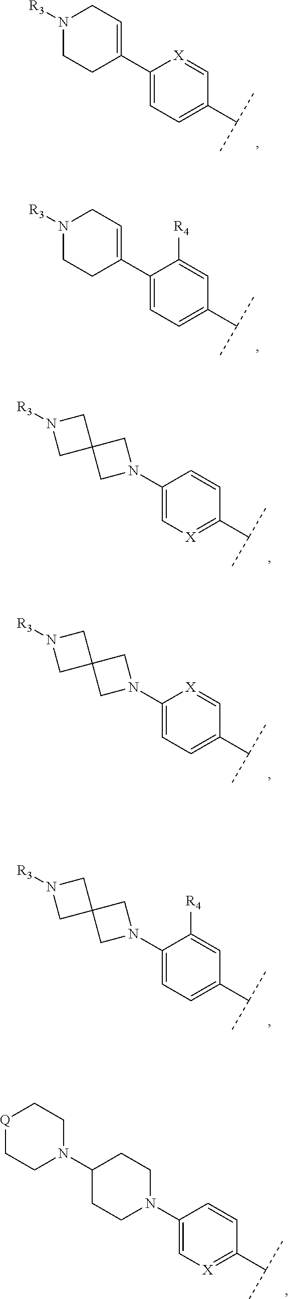

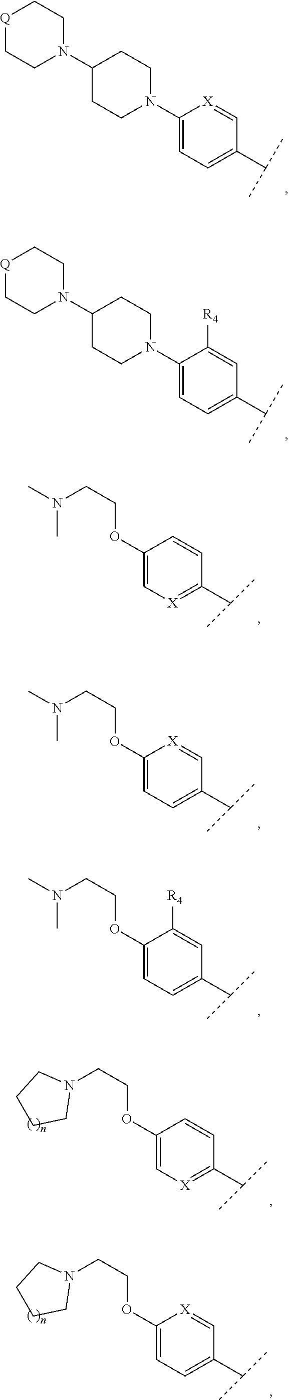

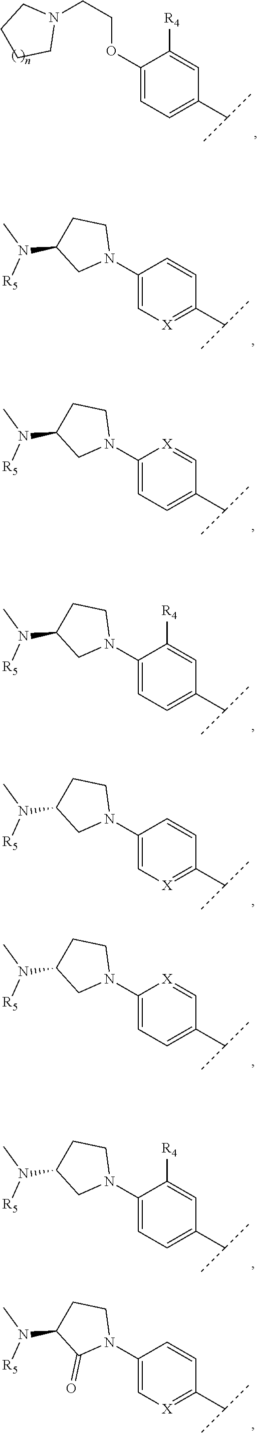

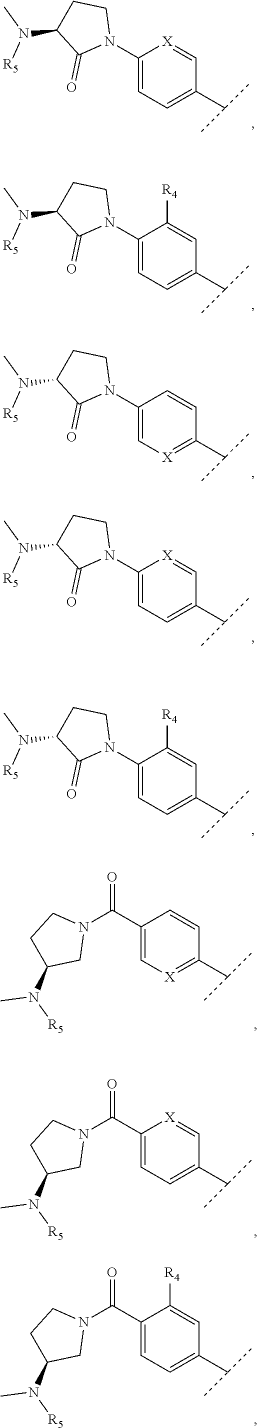

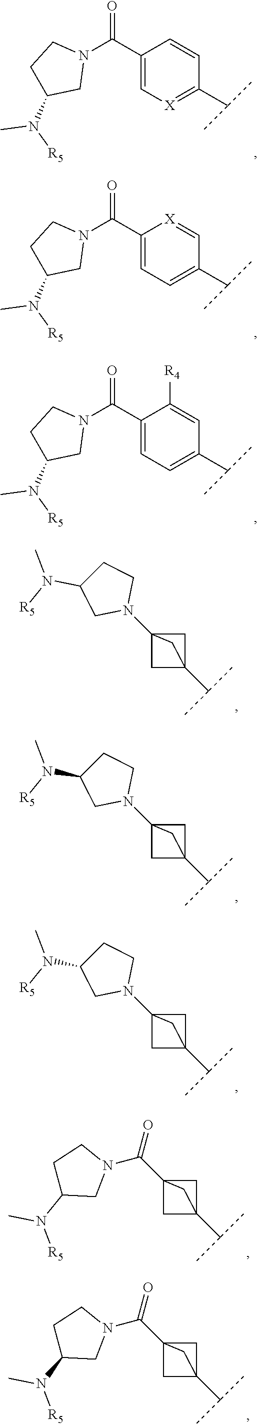

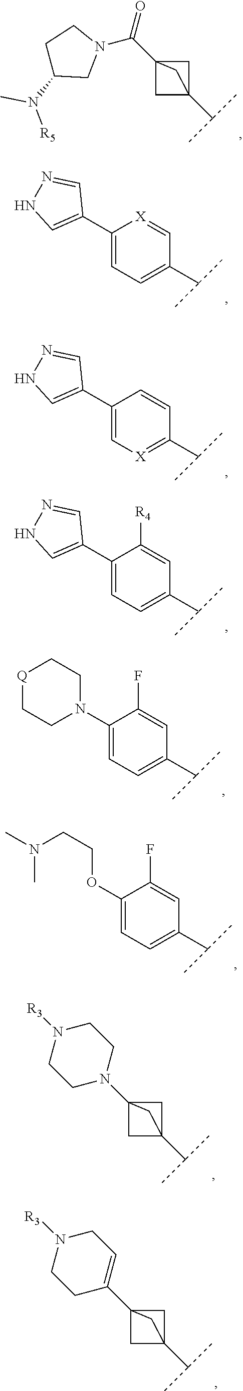

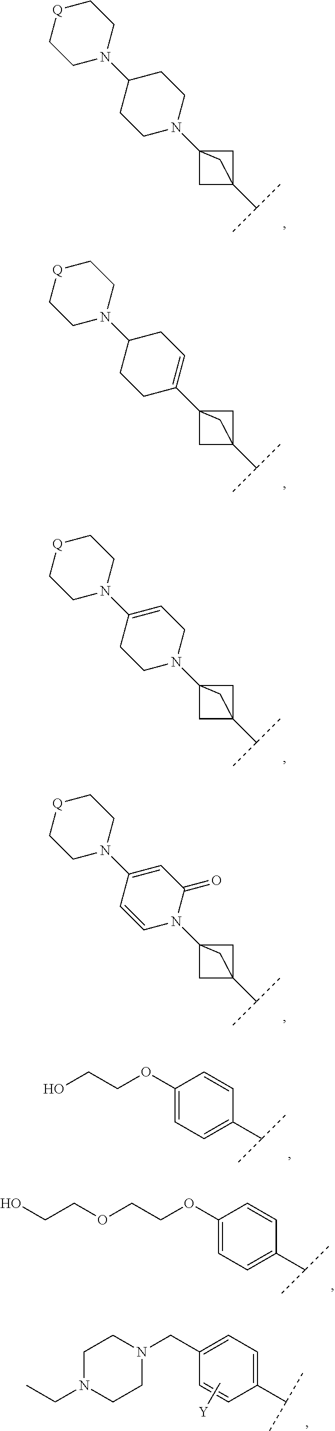

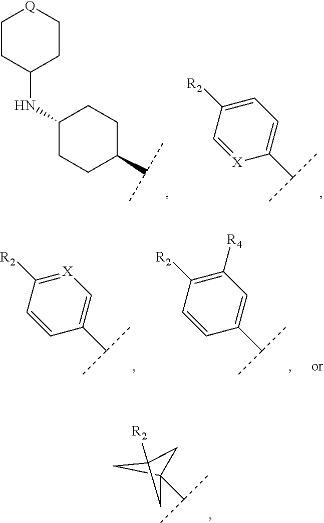

where R₃ is H, Me, isopropyl, isobutyl, CH₂CH₂OH, or bicyclo[1.1.1]pentane;
R₄ is H, Me, OMe, CF₃, or CHF₂;

### R₅ is H or Me;

### X is C—H, N, or C—F;

Y is H, N, F, Me, OMe, CF₃, or CHF₂;

### Q is O or S;

n is 1, 2, or 3; and

### R₂ is:

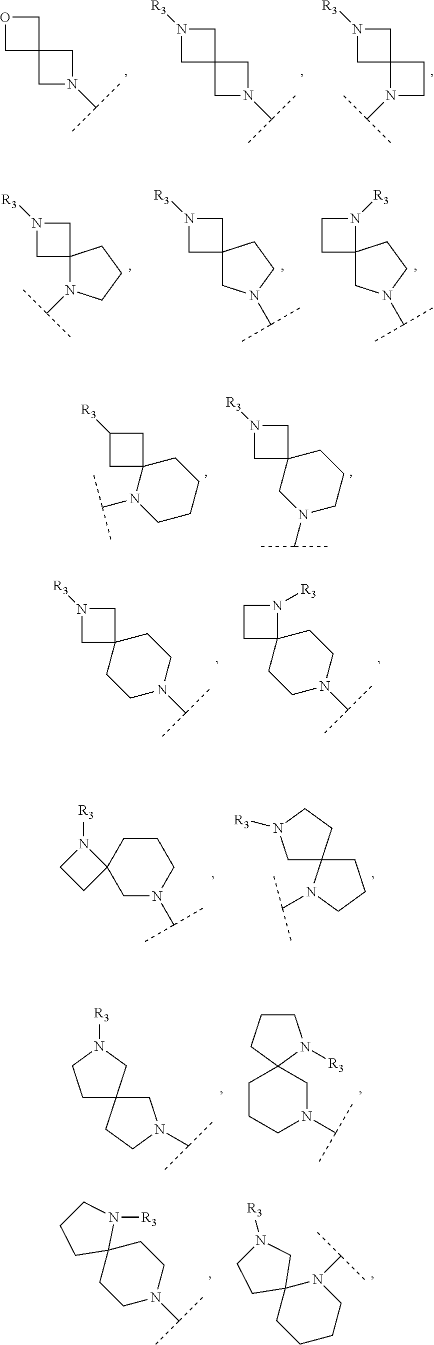

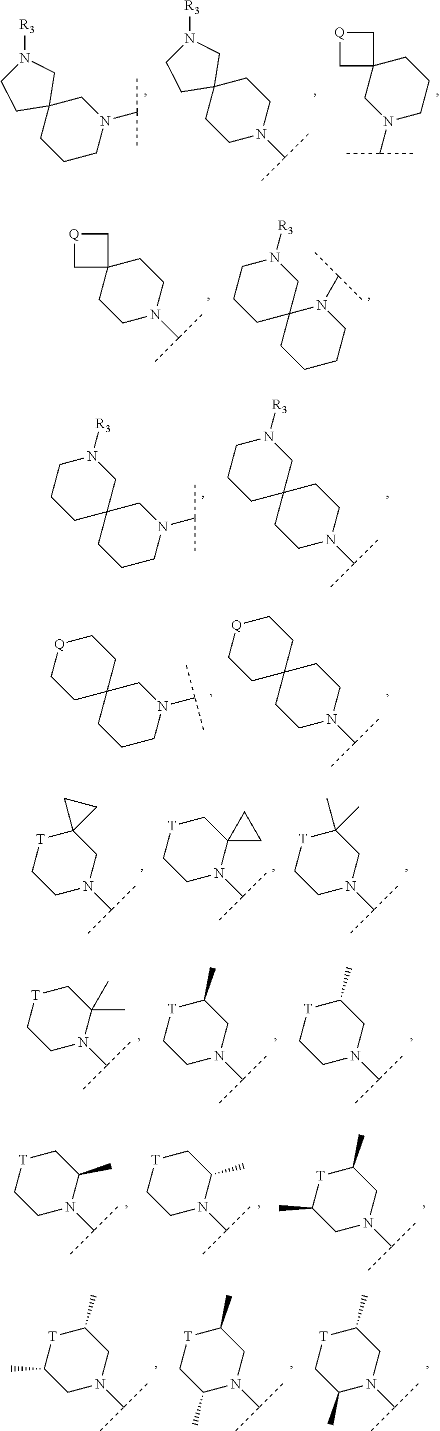

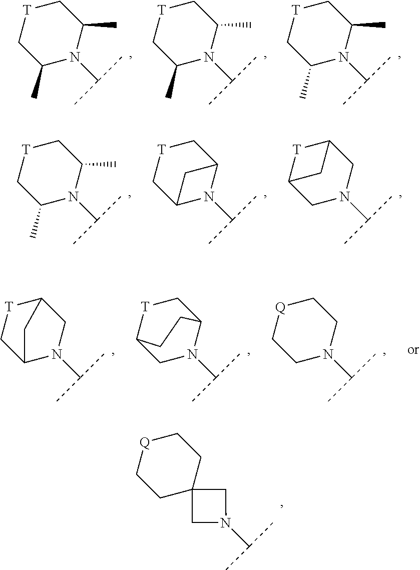

where T is O, NH, NMe, N-isopropyl, or N-isobutyl. In another aspect, the compound comprises Formulae (IX), (XIII), or (XV):

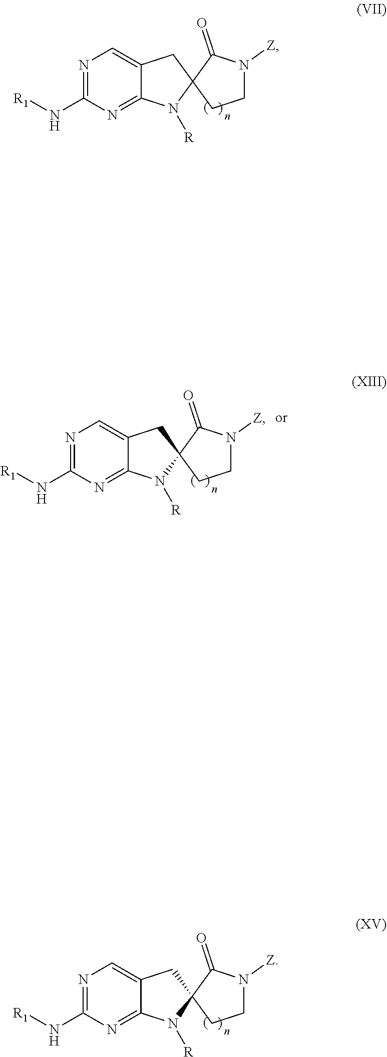

In another aspect, R is

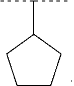

In another aspect, R₁ is:

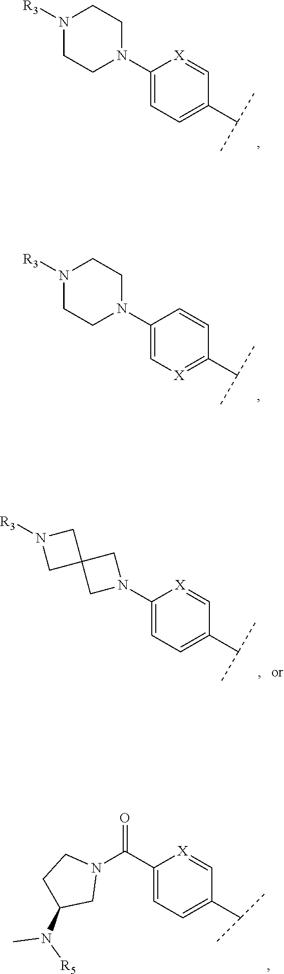

where R₃ is H, Me, isopropyl, isobutyl, CH₂CH₂OH, or bicyclo[1.1.1]pentane; R₅ is H or Me; and X is C—H, N, or C—F.

Another embodiment described herein is a compound selected from C1-C212 or pharmaceutically acceptable salts thereof:

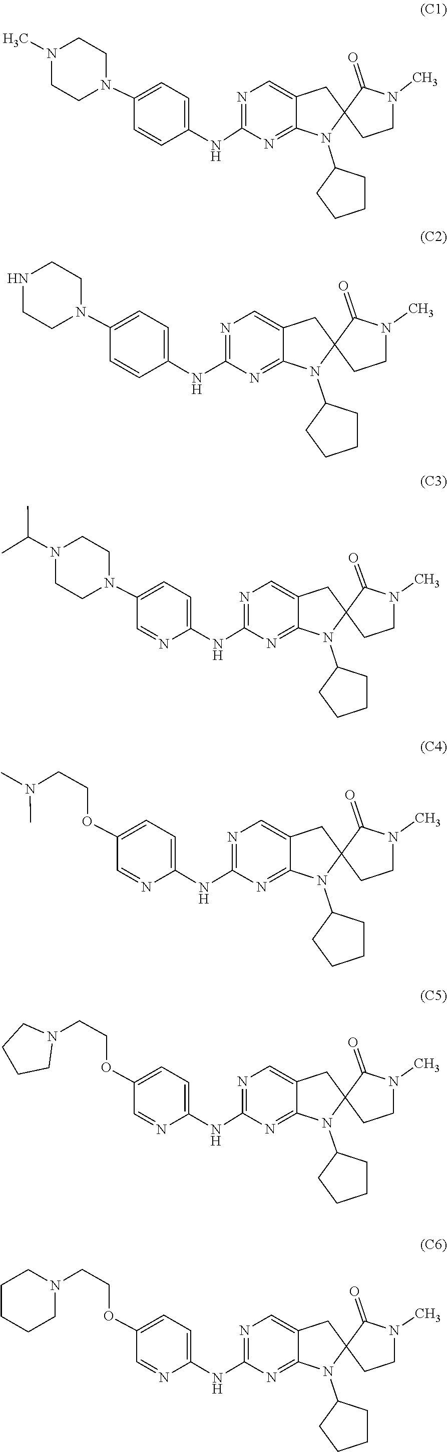

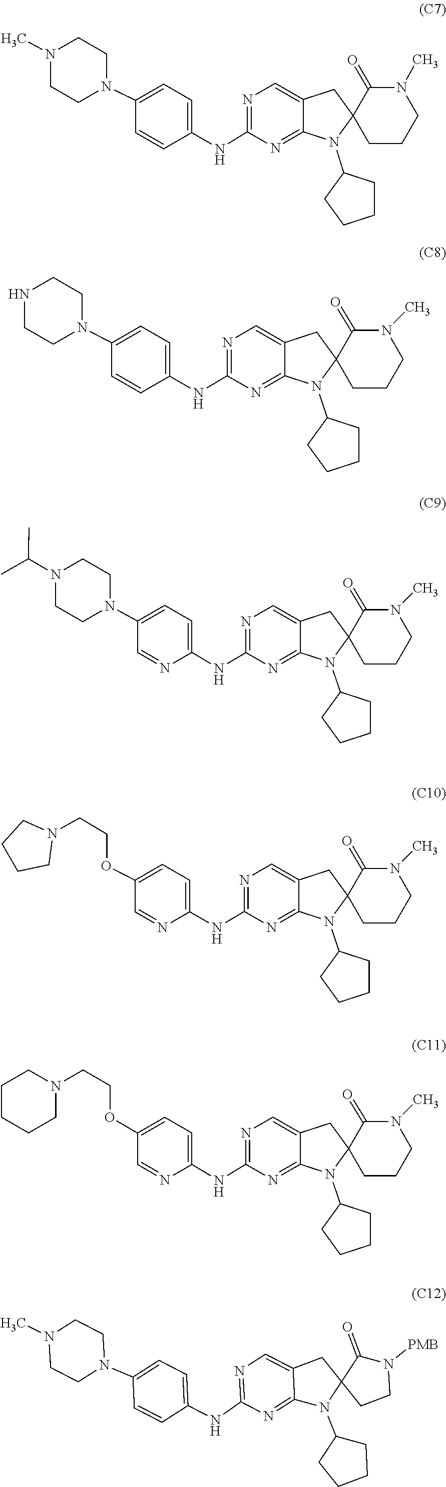

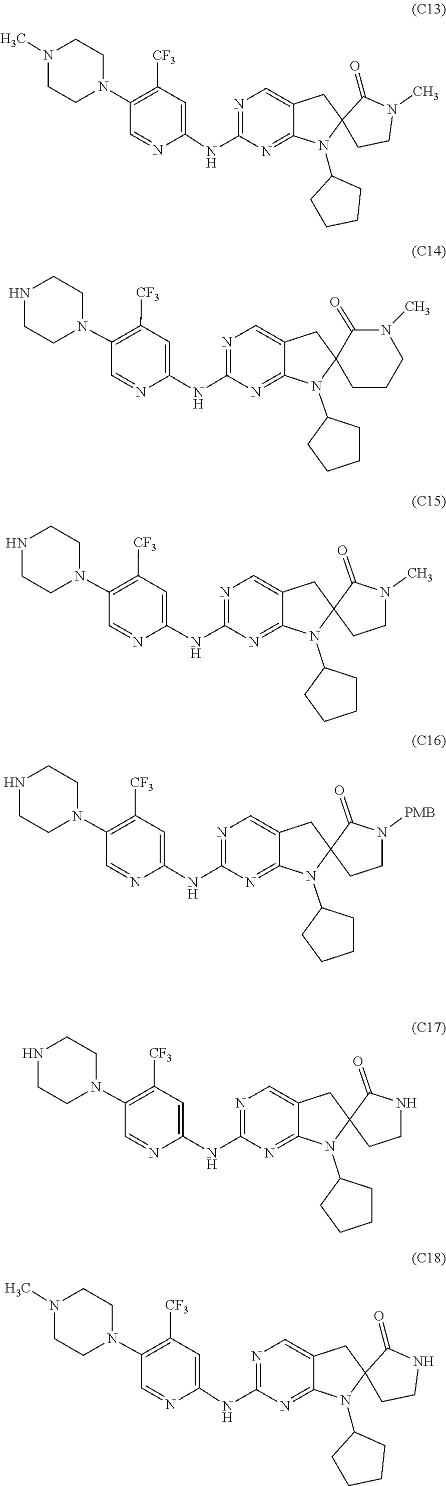

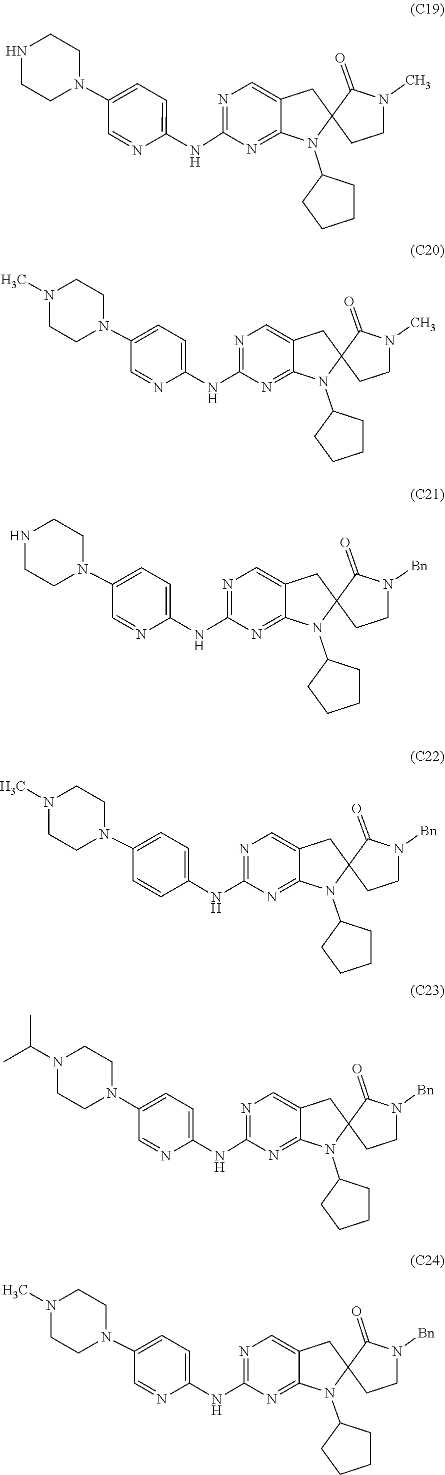

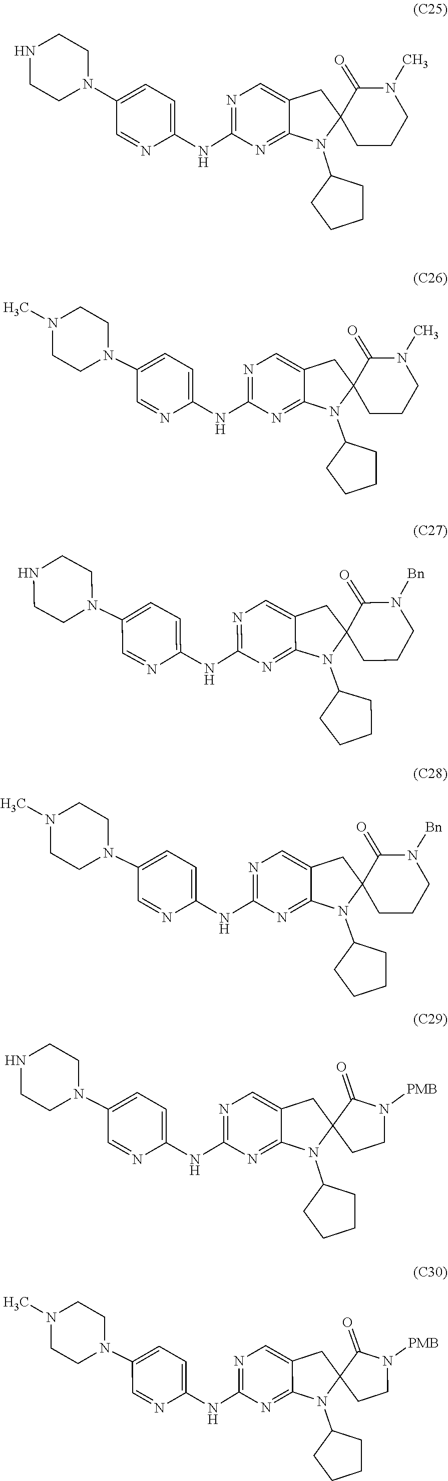

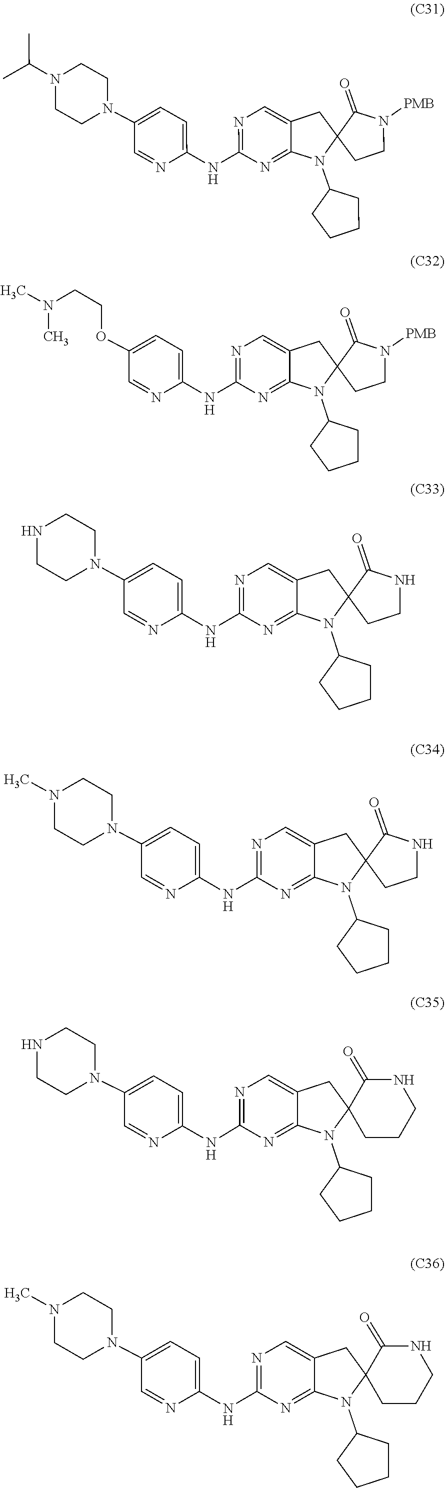

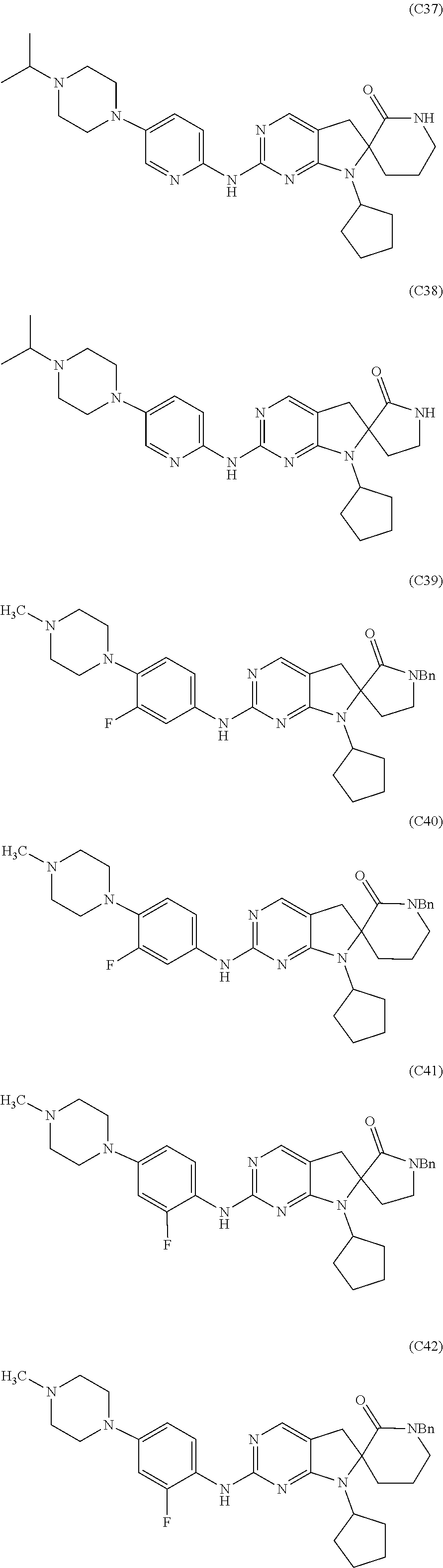 [SMILES: CC(C)N1CCN(c2ccc(Nc3ncc4c(n3)N(C3CCCC3)C3(CCCNC3=O)C4)nc2)CC1 ; CC(C)N1CCN(c2ccc(Nc3ncc4c(n3)N(C3CCCC3)C3(CCNC3=O)C4)nc2)CC1 ; CN1CCN(c2ccc(Nc3ncc4c(n3)N(C3CCCC3)C3(CCN(Cc5ccccc5)C3=O)C4)cc2F)CC1 ; CN1CCN(c2ccc(Nc3ncc4c(n3)N(C3CCCC3)C3(CCCN(Cc5ccccc5)C3=O)C4)cc2F)CC1 ; CN1CCN(c2ccc(Nc3ncc4c(n3)N(C3CCCC3)C3(CCN(Cc5ccccc5)C3=O)C4)c(F)c2)CC1 ; CN1CCN(c2ccc(Nc3ncc4c(n3)N(C3CCCC3)C3(CCCN(Cc5ccccc5)C3=O)C4)c(F)c2)CC1]

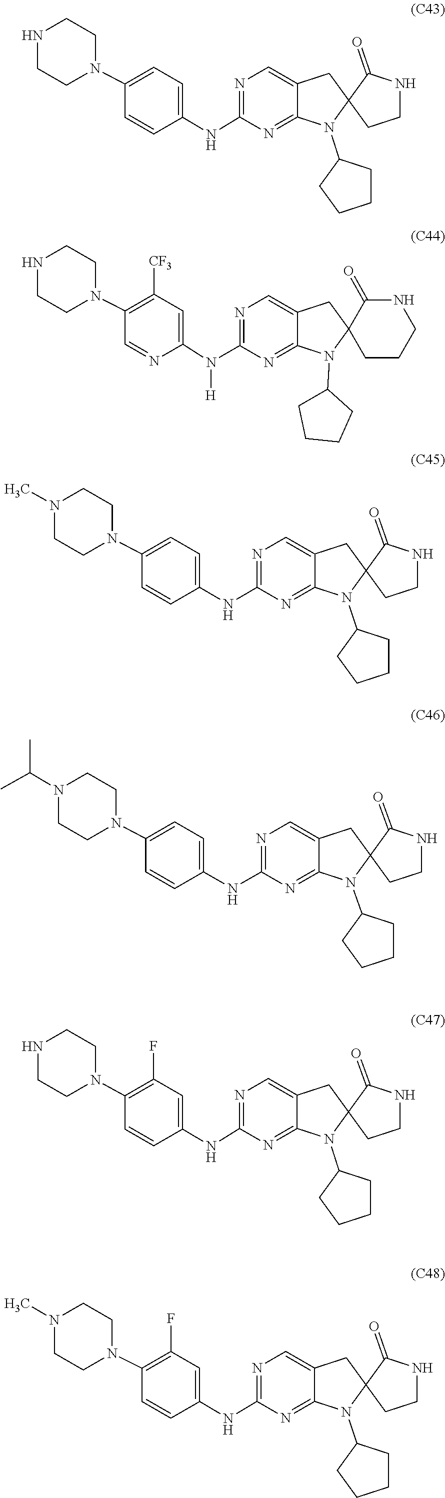 [SMILES: O=C1NCCC12Cc1cnc(Nc3ccc(N4CCNCC4)cc3)nc1N2C1CCCC1 ; O=C1NCCCC12Cc1cnc(Nc3cc(C(F)(F)F)c(N4CCNCC4)cn3)nc1N2C1CCCC1 ; CN1CCN(c2ccc(Nc3ncc4c(n3)N(C3CCCC3)C3(CCNC3=O)C4)cc2)CC1 ; CC(C)N1CCN(c2ccc(Nc3ncc4c(n3)N(C3CCCC3)C3(CCNC3=O)C4)cc2)CC1 ; O=C1NCCC12Cc1cnc(Nc3ccc(N4CCNCC4)c(F)c3)nc1N2C1CCCC1 ; CN1CCN(c2ccc(Nc3ncc4c(n3)N(C3CCCC3)C3(CCNC3=O)C4)cc2F)CC1]

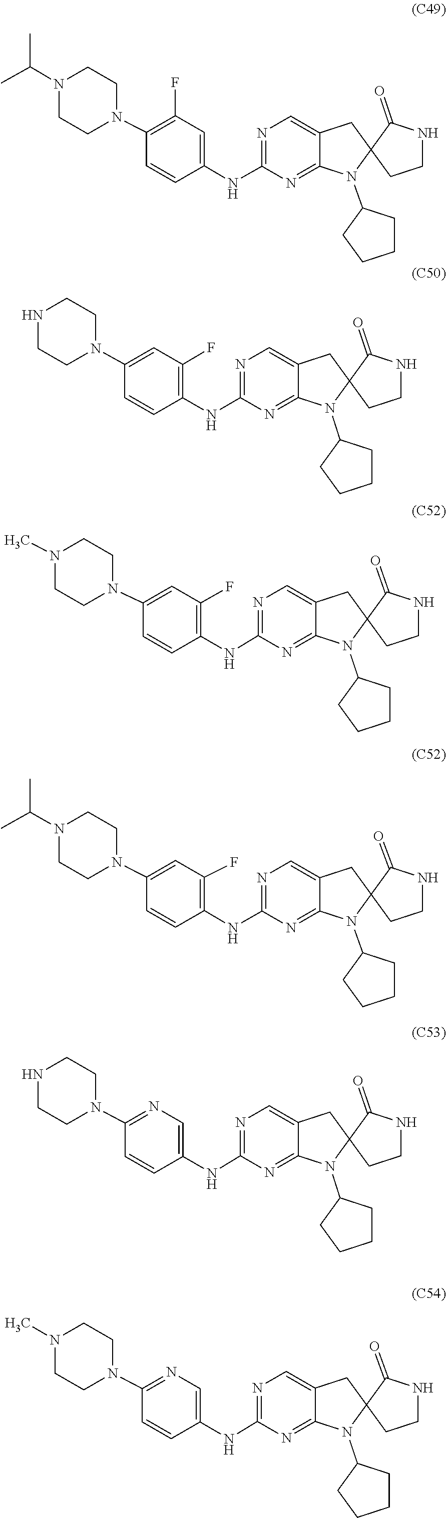 [SMILES: CC(C)N1CCN(c2ccc(Nc3ncc4c(n3)N(C3CCCC3)C3(CCNC3=O)C4)cc2F)CC1 ; O=C1NCCC12Cc1cnc(Nc3ccc(N4CCNCC4)cc3F)nc1N2C1CCCC1 ; CN1CCN(c2ccc(Nc3ncc4c(n3)N(C3CCCC3)C3(CCNC3=O)C4)c(F)c2)CC1 ; CC(C)N1CCN(c2ccc(Nc3ncc4c(n3)N(C3CCCC3)C3(CCNC3=O)C4)c(F)c2)CC1 ; O=C1NCCC12Cc1cnc(Nc3ccc(N4CCNCC4)nc3)nc1N2C1CCCC1 ; CN1CCN(c2ccc(Nc3ncc4c(n3)N(C3CCCC3)C3(CCNC3=O)C4)cn2)CC1]

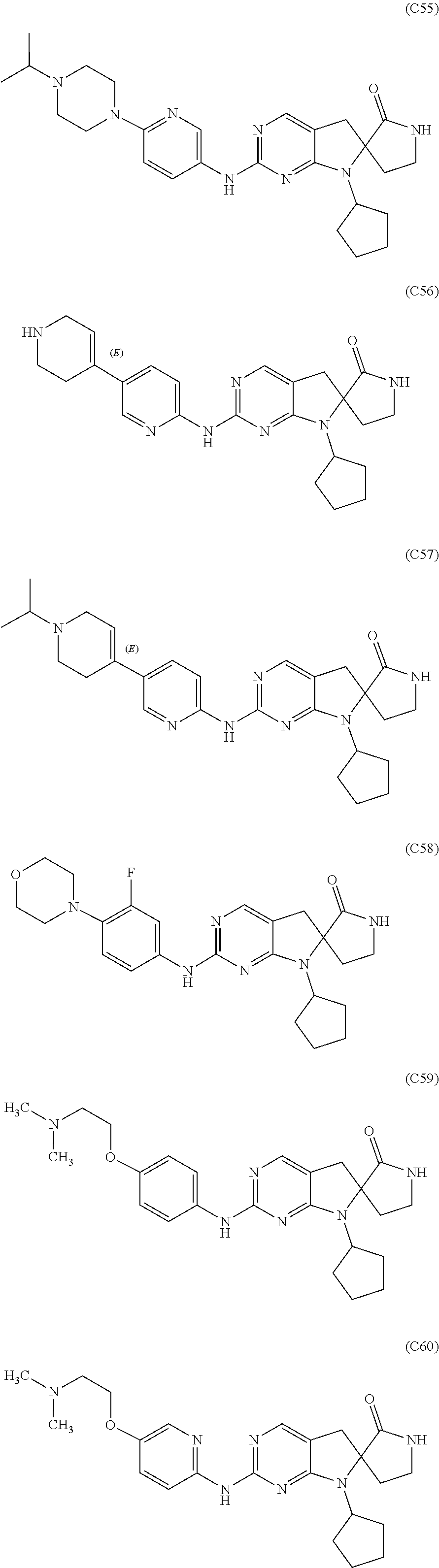 [SMILES: CC(C)N1CCN(c2ccc(Nc3ncc4c(n3)N(C3CCCC3)C3(CCNC3=O)C4)cn2)CC1 ; O=C1NCCC12Cc1cnc(Nc3ccc(C4=CCNCC4)cn3)nc1N2C1CCCC1 ; CC(C)N1CC=C(c2ccc(Nc3ncc4c(n3)N(C3CCCC3)C3(CCNC3=O)C4)nc2)CC1 ; O=C1NCCC12Cc1cnc(Nc3ccc(N4CCOCC4)c(F)c3)nc1N2C1CCCC1 ; CN(C)CCOc1ccc(Nc2ncc3c(n2)N(C2CCCC2)C2(CCNC2=O)C3)cc1 ; CN(C)CCOc1ccc(Nc2ncc3c(n2)N(C2CCCC2)C2(CCNC2=O)C3)nc1]

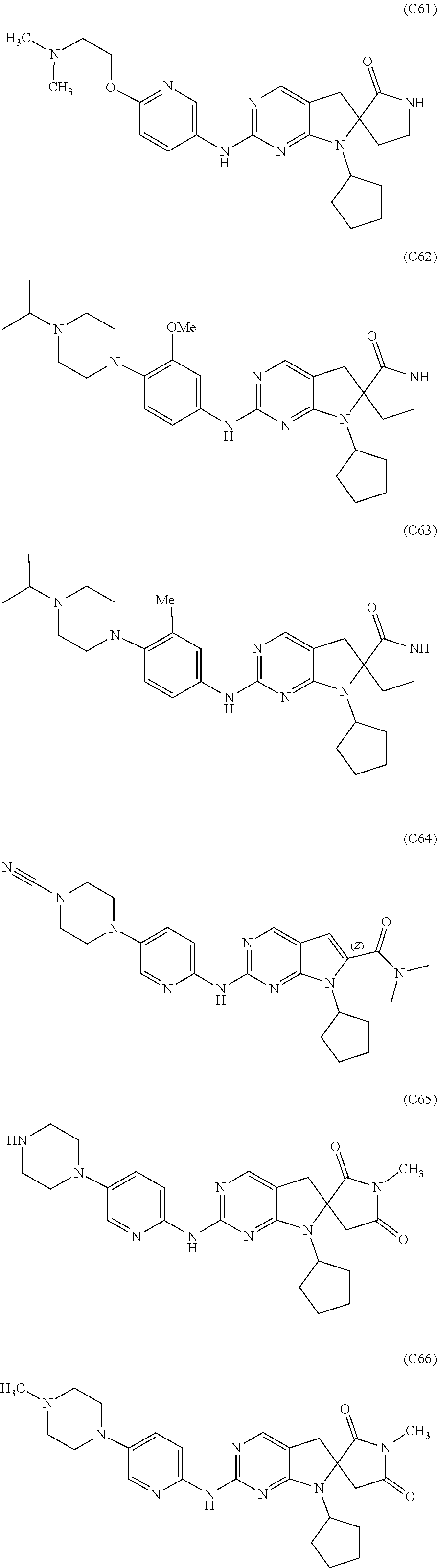

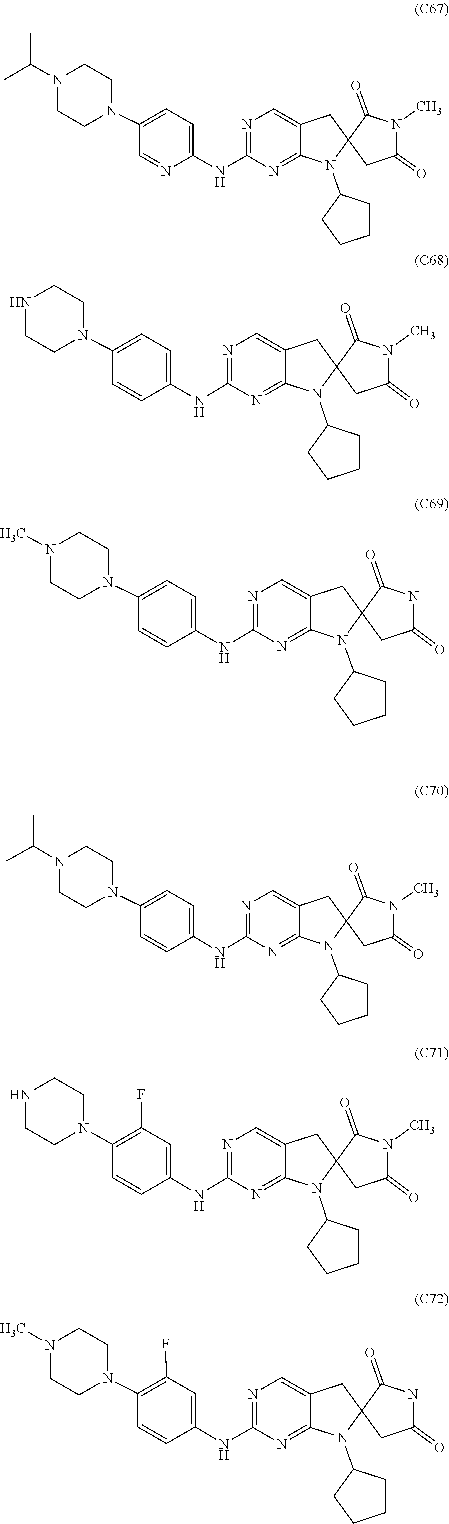

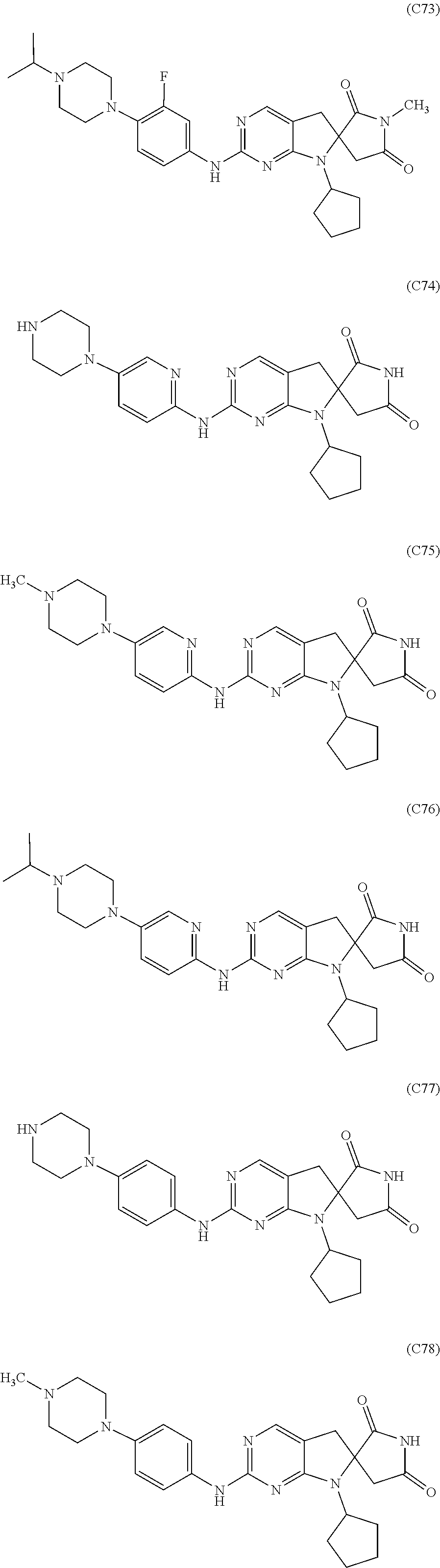

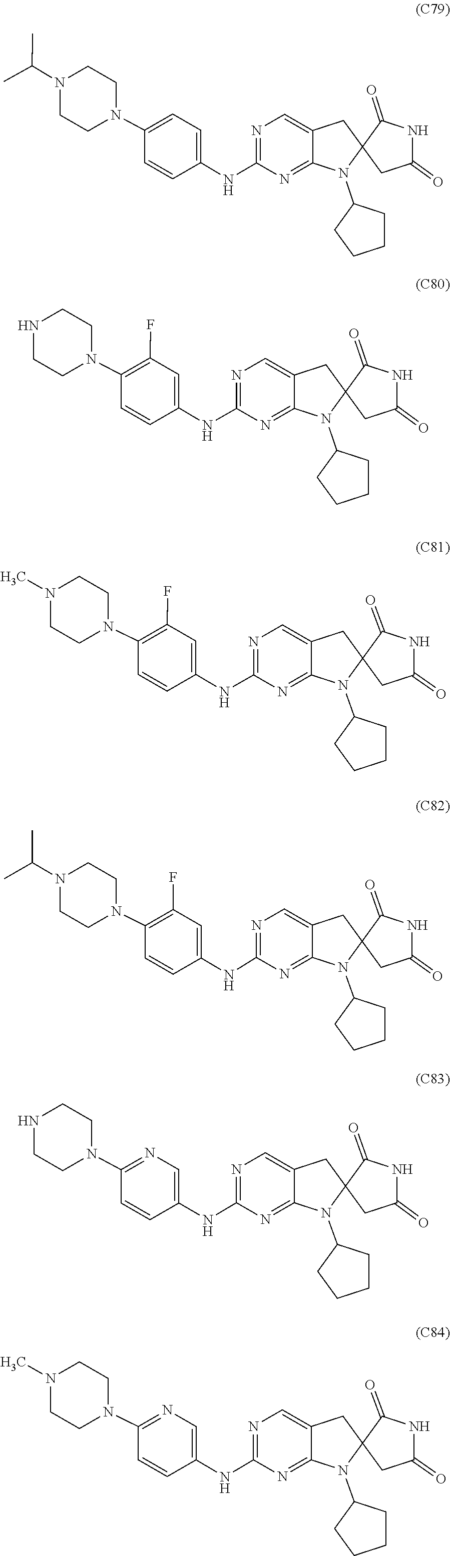 [SMILES: CC(C)N1CCN(c2ccc(Nc3ncc4c(n3)N(C3CCCC3)C3(CC(=O)NC3=O)C4)cc2)CC1 ; O=C1CC2(Cc3cnc(Nc4ccc(N5CCNCC5)c(F)c4)nc3N2C2CCCC2)C(=O)N1 ; CN1CCN(c2ccc(Nc3ncc4c(n3)N(C3CCCC3)C3(CC(=O)NC3=O)C4)cc2F)CC1 ; CC(C)N1CCN(c2ccc(Nc3ncc4c(n3)N(C3CCCC3)C3(CC(=O)NC3=O)C4)cc2F)CC1 ; O=C1CC2(Cc3cnc(Nc4ccc(N5CCNCC5)nc4)nc3N2C2CCCC2)C(=O)N1 ; CN1CCN(c2ccc(Nc3ncc4c(n3)N(C3CCCC3)C3(CC(=O)NC3=O)C4)cn2)CC1]

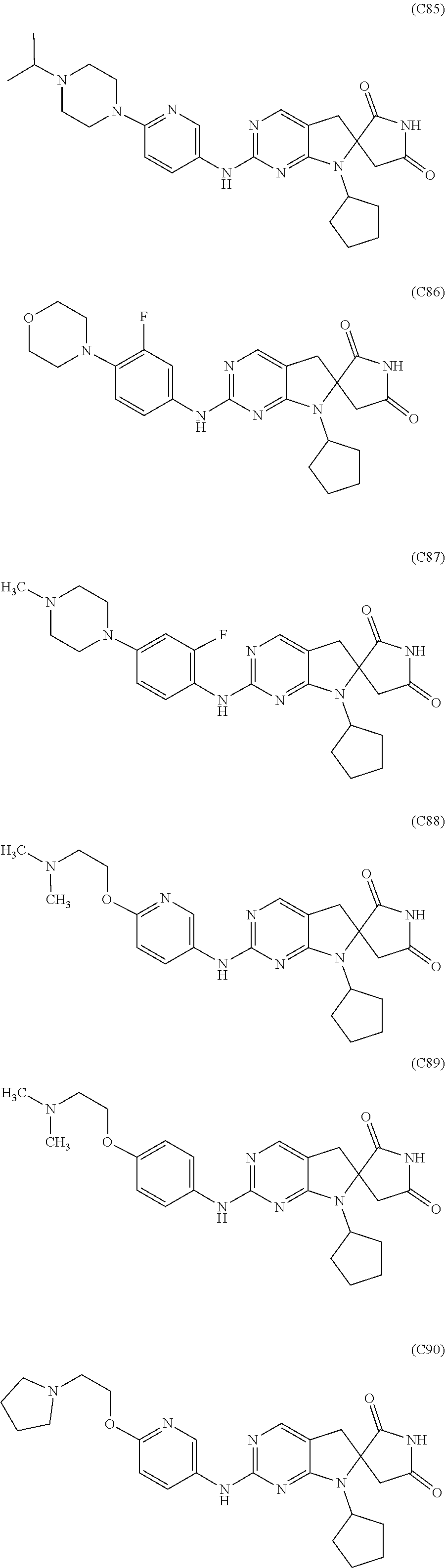 [SMILES: CC(C)N1CCN(c2ccc(Nc3ncc4c(n3)N(C3CCCC3)C3(CC(=O)NC3=O)C4)cn2)CC1 ; O=C1CC2(Cc3cnc(Nc4ccc(N5CCOCC5)c(F)c4)nc3N2C2CCCC2)C(=O)N1 ; CN1CCN(c2ccc(Nc3ncc4c(n3)N(C3CCCC3)C3(CC(=O)NC3=O)C4)c(F)c2)CC1 ; CN(C)CCOc1ccc(Nc2ncc3c(n2)N(C2CCCC2)C2(CC(=O)NC2=O)C3)cn1 ; CN(C)CCOc1ccc(Nc2ncc3c(n2)N(C2CCCC2)C2(CC(=O)NC2=O)C3)cc1 ; O=C1CC2(Cc3cnc(Nc4ccc(OCCN5CCCC5)nc4)nc3N2C2CCCC2)C(=O)N1]

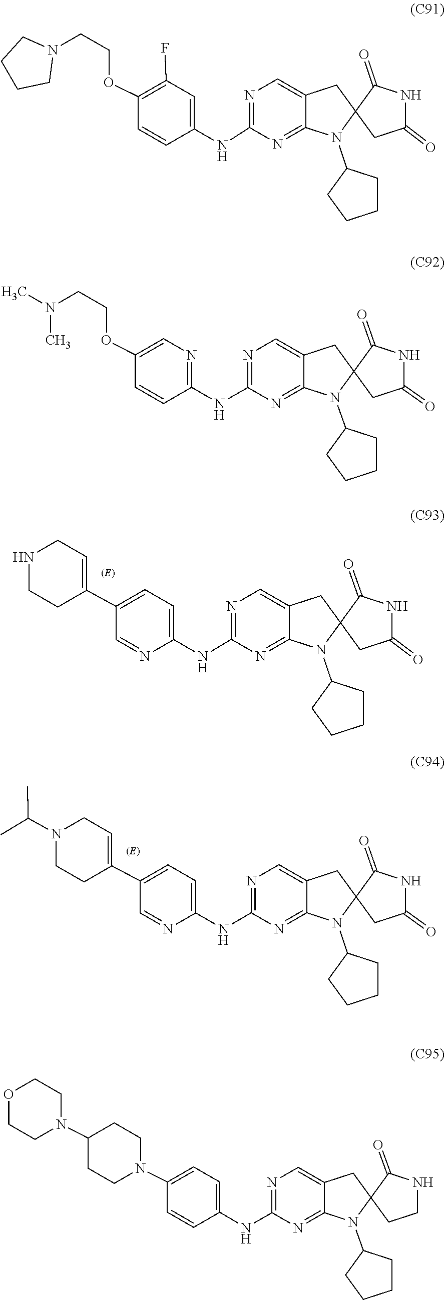 [SMILES: O=C1CC2(Cc3cnc(Nc4ccc(OCCN5CCCC5)c(F)c4)nc3N2C2CCCC2)C(=O)N1 ; CN(C)CCOc1ccc(Nc2ncc3c(n2)N(C2CCCC2)C2(CC(=O)NC2=O)C3)nc1 ; O=C1CC2(Cc3cnc(Nc4ccc(C5=CCNCC5)cn4)nc3N2C2CCCC2)C(=O)N1 ; CC(C)N1CC=C(c2ccc(Nc3ncc4c(n3)N(C3CCCC3)C3(CC(=O)NC3=O)C4)nc2)CC1 ; O=C1NCCC12Cc1cnc(Nc3ccc(N4CCC(N5CCOCC5)CC4)cc3)nc1N2C1CCCC1]

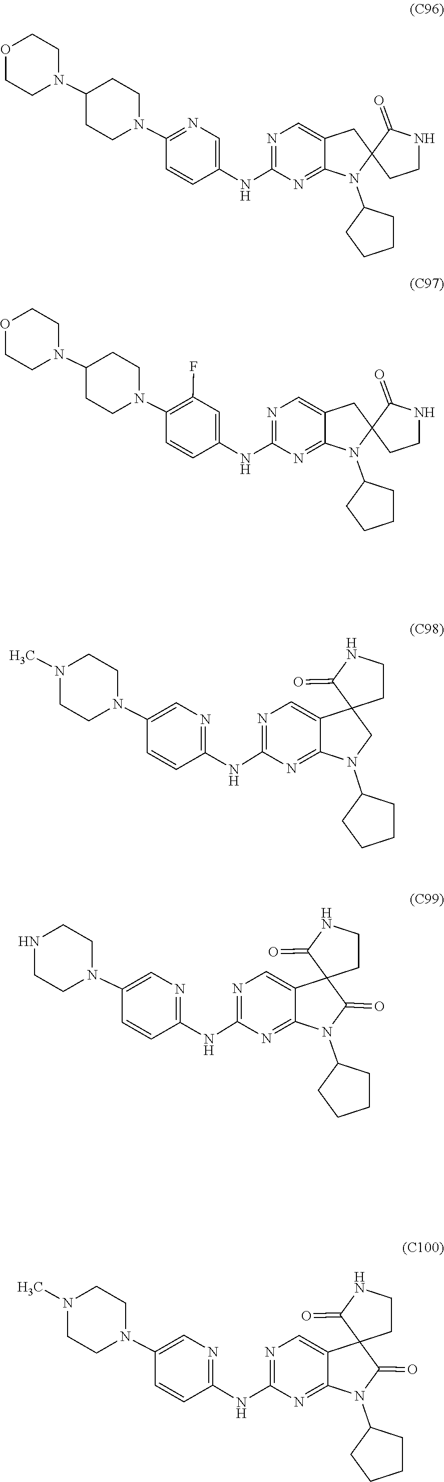 [SMILES: O=C1NCCC12Cc1cnc(Nc3ccc(N4CCC(N5CCOCC5)CC4)nc3)nc1N2C1CCCC1 ; O=C1NCCC12Cc1cnc(Nc3ccc(N4CCC(N5CCOCC5)CC4)c(F)c3)nc1N2C1CCCC1 ; CN1CCN(c2ccc(Nc3ncc4c(n3)N(C3CCCC3)CC43CCNC3=O)nc2)CC1 ; O=C1NCCC12C(=O)N(C1CCCC1)c1nc(Nc3ccc(N4CCNCC4)cn3)ncc12 ; CN1CCN(c2ccc(Nc3ncc4c(n3)N(C3CCCC3)C(=O)C43CCNC3=O)nc2)CC1]

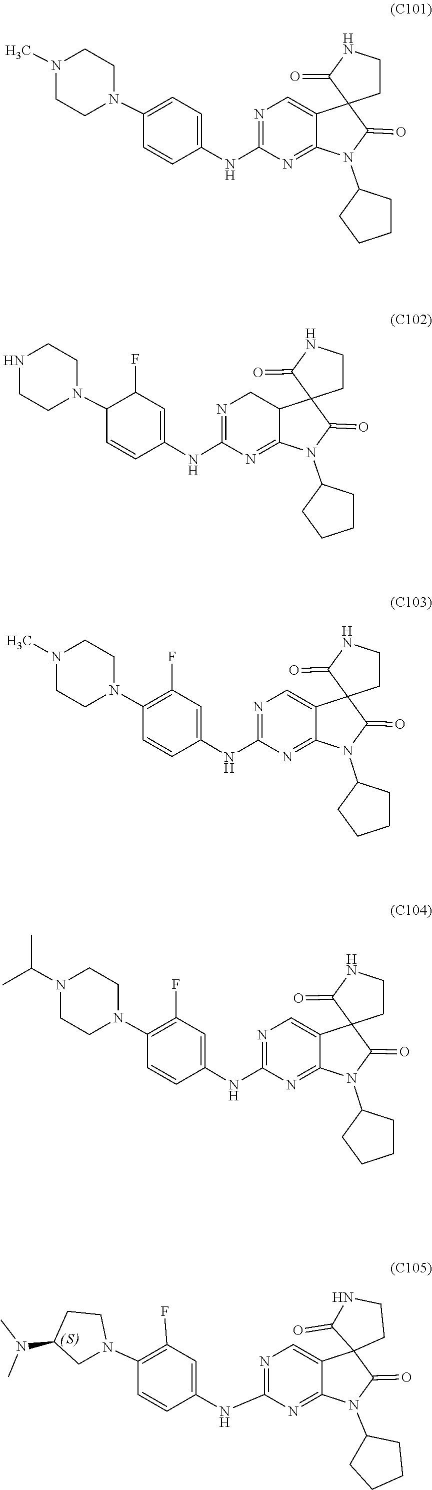

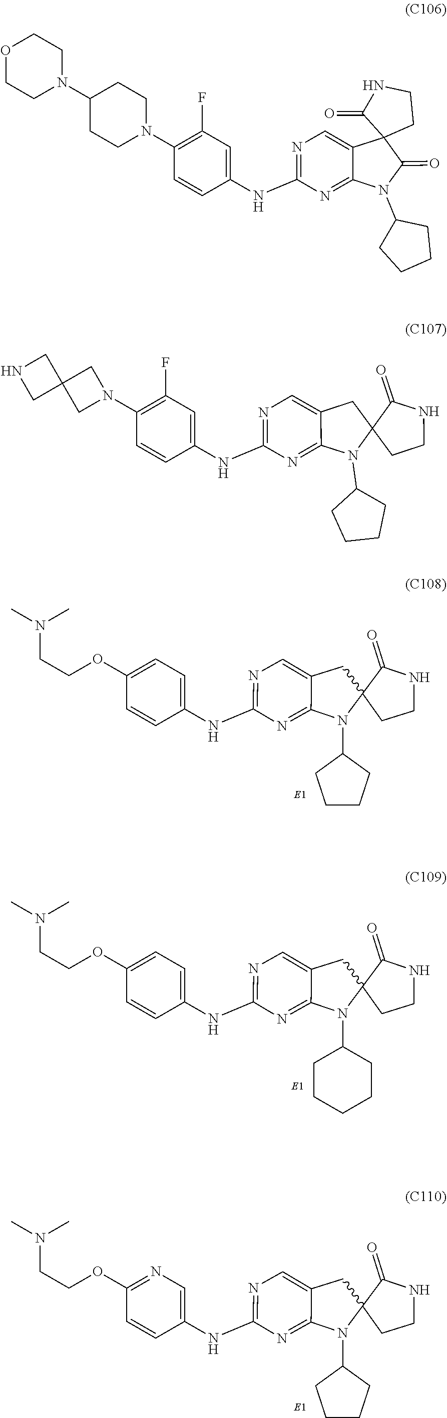 [SMILES: O=C1NCCC12C(=O)N(C1CCCC1)c1nc(Nc3ccc(N4CCC(N5CCOCC5)CC4)c(F)c3)ncc12 ; O=C1NCCC12Cc1cnc(Nc3ccc(N4CC5(CNC5)C4)c(F)c3)nc1N2C1CCCC1 ; CN(C)CCOc1ccc(Nc2ncc3c(n2)N(C2CCCC2)C2(CCNC2=O)C3)cc1 ; CN(C)CCOc1ccc(Nc2ncc3c(n2)N(C2CCCCC2)C2(CCNC2=O)C3)cc1 ; CN(C)CCOc1ccc(Nc2ncc3c(n2)N(C2CCCC2)C2(CCNC2=O)C3)cn1]

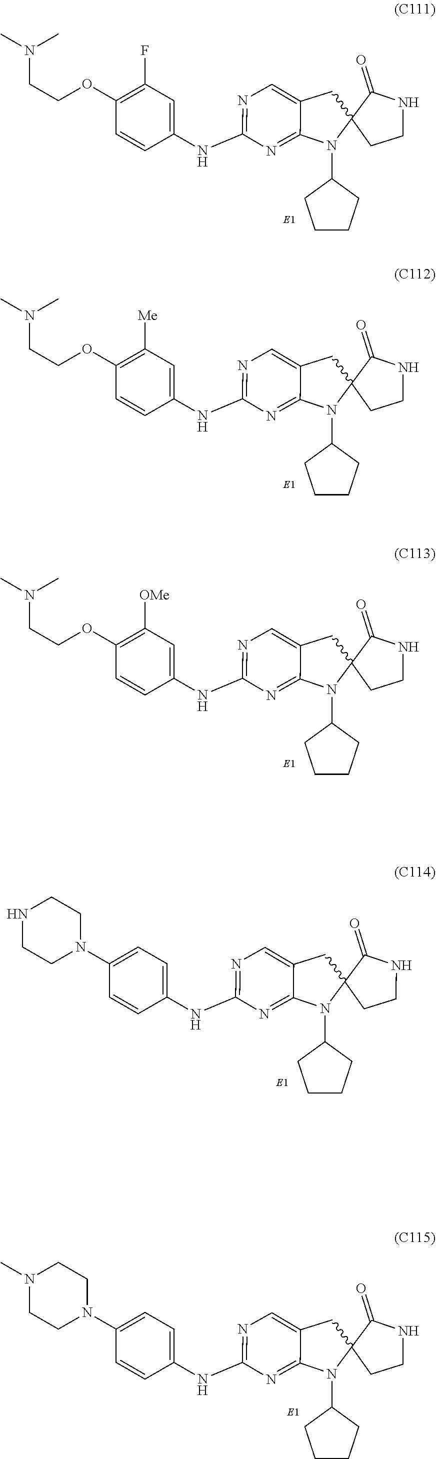 [SMILES: CN(C)CCOc1ccc(Nc2ncc3c(n2)N(C2CCCC2)C2(CCNC2=O)C3)cc1F ; Cc1cc(Nc2ncc3c(n2)N(C2CCCC2)C2(CCNC2=O)C3)ccc1OCCN(C)C ; COc1cc(Nc2ncc3c(n2)N(C2CCCC2)C2(CCNC2=O)C3)ccc1OCCN(C)C ; O=C1NCCC12Cc1cnc(Nc3ccc(N4CCNCC4)cc3)nc1N2C1CCCC1 ; CN1CCN(c2ccc(Nc3ncc4c(n3)N(C3CCCC3)C3(CCNC3=O)C4)cc2)CC1]

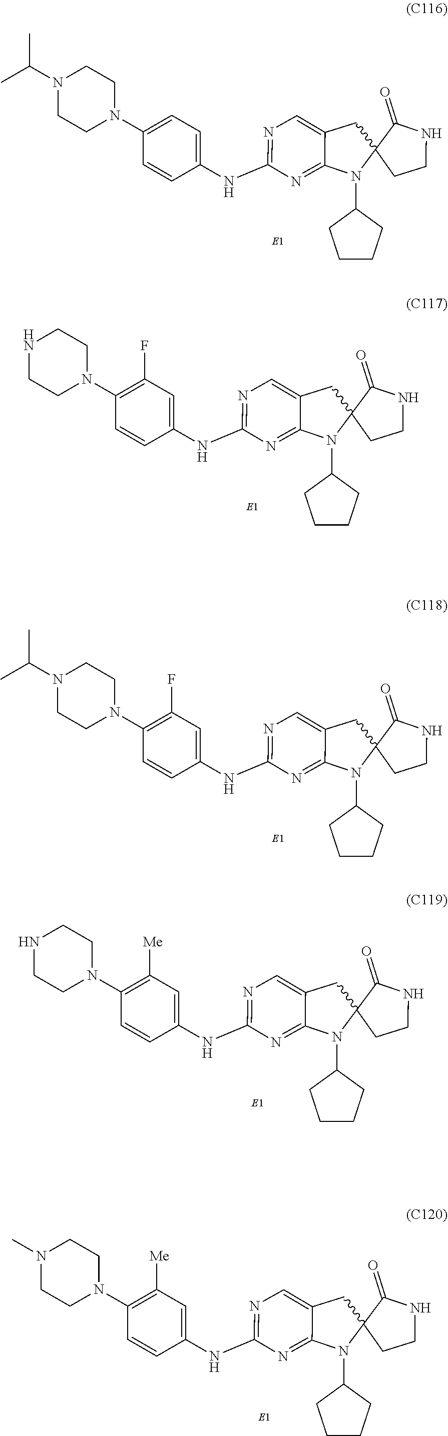 [SMILES: CC(C)N1CCN(c2ccc(Nc3ncc4c(n3)N(C3CCCC3)C3(CCNC3=O)C4)cc2)CC1 ; O=C1NCCC12Cc1cnc(Nc3ccc(N4CCNCC4)c(F)c3)nc1N2C1CCCC1 ; CC(C)N1CCN(c2ccc(Nc3ncc4c(n3)N(C3CCCC3)C3(CCNC3=O)C4)cc2F)CC1 ; Cc1cc(Nc2ncc3c(n2)N(C2CCCC2)C2(CCNC2=O)C3)ccc1N1CCNCC1 ; Cc1cc(Nc2ncc3c(n2)N(C2CCCC2)C2(CCNC2=O)C3)ccc1N1CCN(C)CC1]

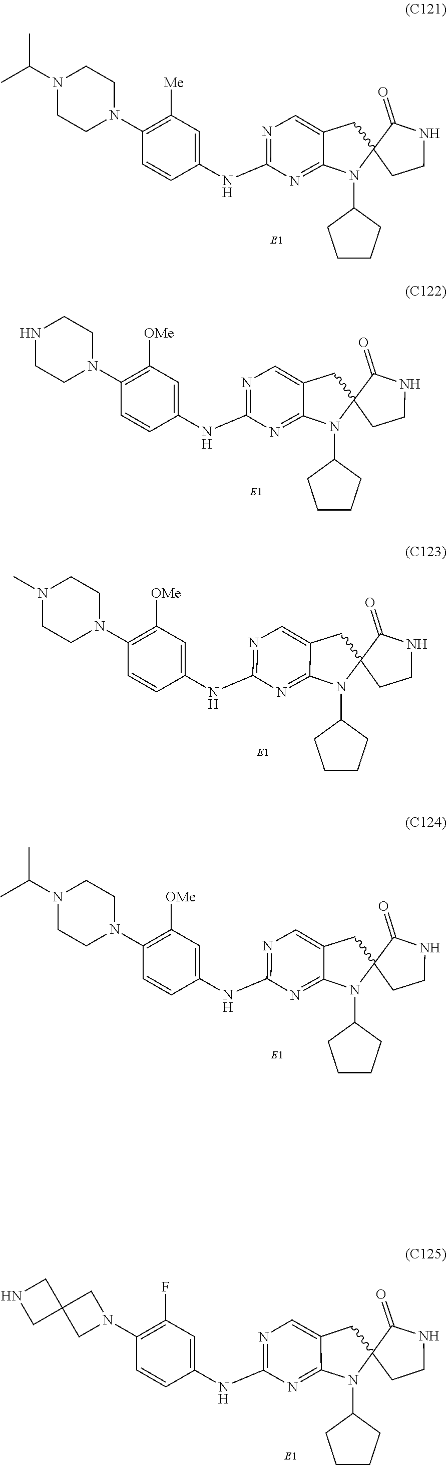 [SMILES: Cc1cc(Nc2ncc3c(n2)N(C2CCCC2)C2(CCNC2=O)C3)ccc1N1CCN(C(C)C)CC1 ; COc1cc(Nc2ncc3c(n2)N(C2CCCC2)C2(CCNC2=O)C3)ccc1N1CCNCC1 ; COc1cc(Nc2ncc3c(n2)N(C2CCCC2)C2(CCNC2=O)C3)ccc1N1CCN(C)CC1 ; COc1cc(Nc2ncc3c(n2)N(C2CCCC2)C2(CCNC2=O)C3)ccc1N1CCN(C(C)C)CC1 ; O=C1NCCC12Cc1cnc(Nc3ccc(N4CC5(CNC5)C4)c(F)c3)nc1N2C1CCCC1]

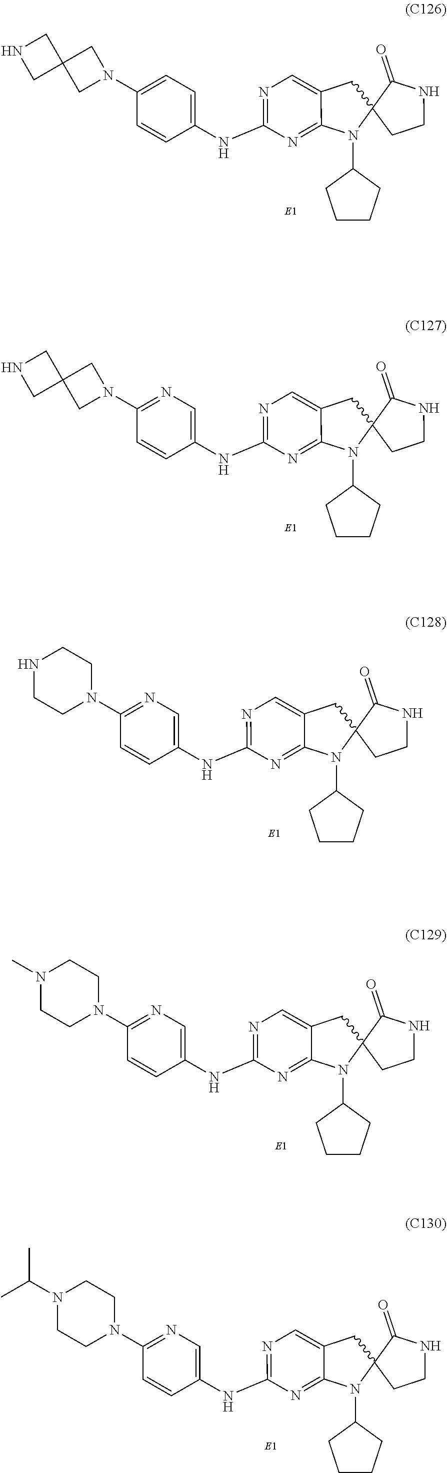 [SMILES: O=C1NCCC12Cc1cnc(Nc3ccc(N4CC5(CNC5)C4)cc3)nc1N2C1CCCC1 ; O=C1NCCC12Cc1cnc(Nc3ccc(N4CC5(CNC5)C4)nc3)nc1N2C1CCCC1 ; O=C1NCCC12Cc1cnc(Nc3ccc(N4CCNCC4)nc3)nc1N2C1CCCC1 ; CN1CCN(c2ccc(Nc3ncc4c(n3)N(C3CCCC3)C3(CCNC3=O)C4)cn2)CC1 ; CC(C)N1CCN(c2ccc(Nc3ncc4c(n3)N(C3CCCC3)C3(CCNC3=O)C4)cn2)CC1]

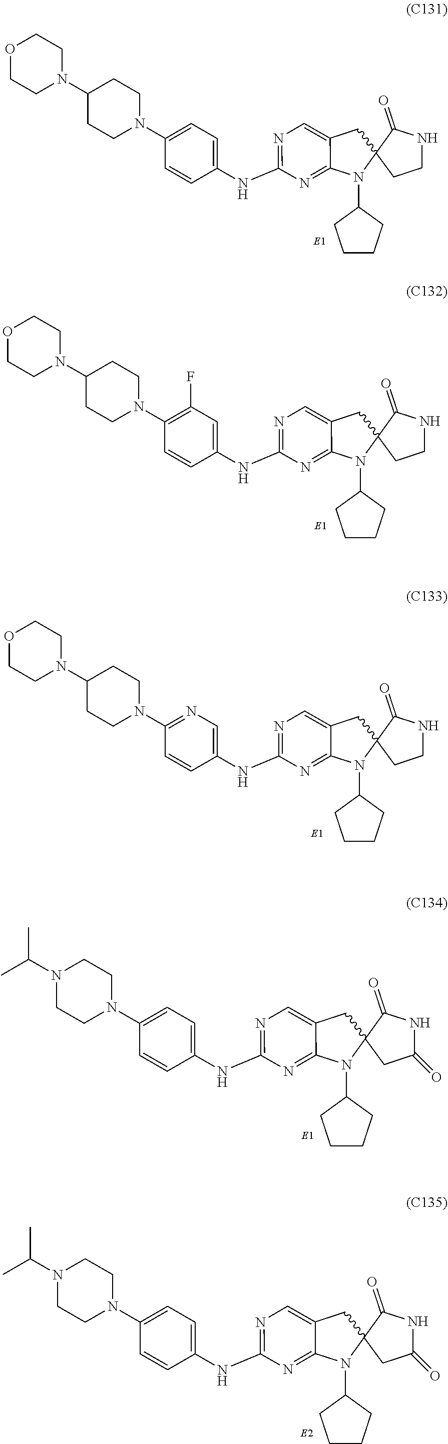 [SMILES: O=C1NCCC12Cc1cnc(Nc3ccc(N4CCC(N5CCOCC5)CC4)cc3)nc1N2C1CCCC1 ; O=C1NCCC12Cc1cnc(Nc3ccc(N4CCC(N5CCOCC5)CC4)c(F)c3)nc1N2C1CCCC1 ; O=C1NCCC12Cc1cnc(Nc3ccc(N4CCC(N5CCOCC5)CC4)nc3)nc1N2C1CCCC1 ; CC(C)N1CCN(c2ccc(Nc3ncc4c(n3)N(C3CCCC3)C3(CC(=O)NC3=O)C4)cc2)CC1 ; CC(C)N1CCN(c2ccc(Nc3ncc4c(n3)N(C3CCCC3)C3(CC(=O)NC3=O)C4)cc2)CC1]

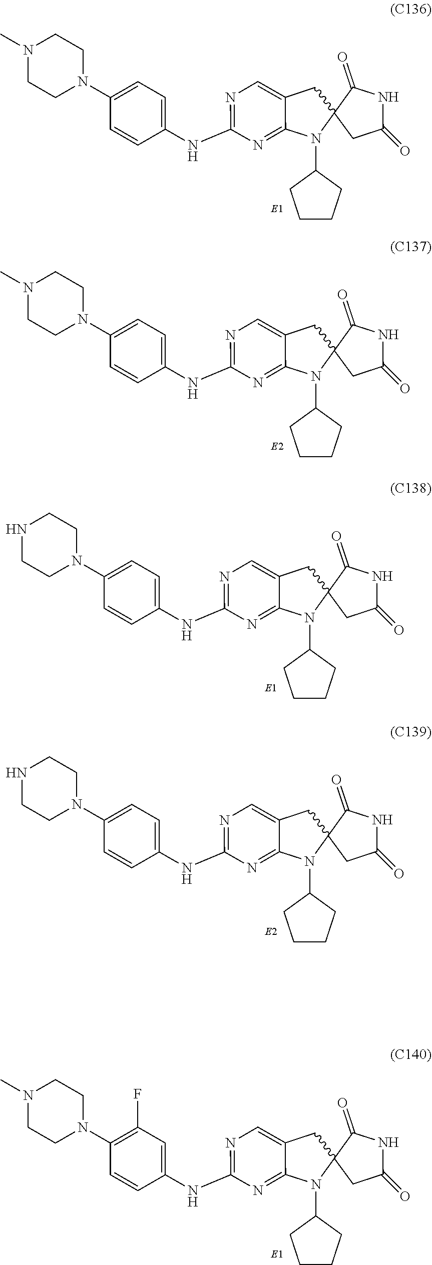 [SMILES: CN1CCN(c2ccc(Nc3ncc4c(n3)N(C3CCCC3)C3(CC(=O)NC3=O)C4)cc2)CC1 ; CN1CCN(c2ccc(Nc3ncc4c(n3)N(C3CCCC3)C3(CC(=O)NC3=O)C4)cc2)CC1 ; O=C1CC2(Cc3cnc(Nc4ccc(N5CCNCC5)cc4)nc3N2C2CCCC2)C(=O)N1 ; O=C1CC2(Cc3cnc(Nc4ccc(N5CCNCC5)cc4)nc3N2C2CCCC2)C(=O)N1 ; CN1CCN(c2ccc(Nc3ncc4c(n3)N(C3CCCC3)C3(CC(=O)NC3=O)C4)cc2F)CC1]

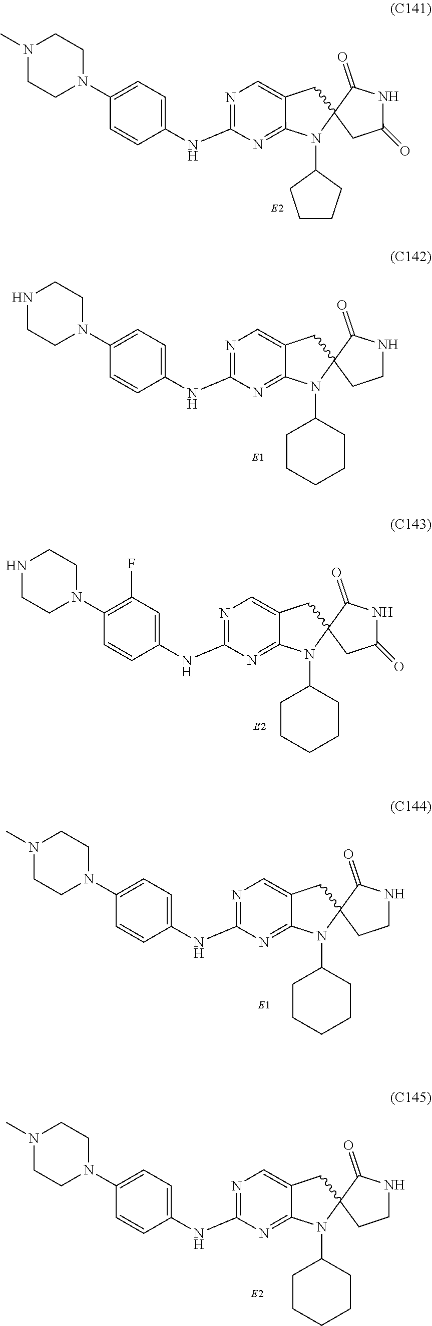 [SMILES: CN1CCN(c2ccc(Nc3ncc4c(n3)N(C3CCCC3)C3(CC(=O)NC3=O)C4)cc2)CC1 ; O=C1NCCC12Cc1cnc(Nc3ccc(N4CCNCC4)cc3)nc1N2C1CCCCC1 ; O=C1CC2(Cc3cnc(Nc4ccc(N5CCNCC5)c(F)c4)nc3N2C2CCCCC2)C(=O)N1 ; CN1CCN(c2ccc(Nc3ncc4c(n3)N(C3CCCCC3)C3(CCNC3=O)C4)cc2)CC1 ; CN1CCN(c2ccc(Nc3ncc4c(n3)N(C3CCCCC3)C3(CCNC3=O)C4)cc2)CC1]

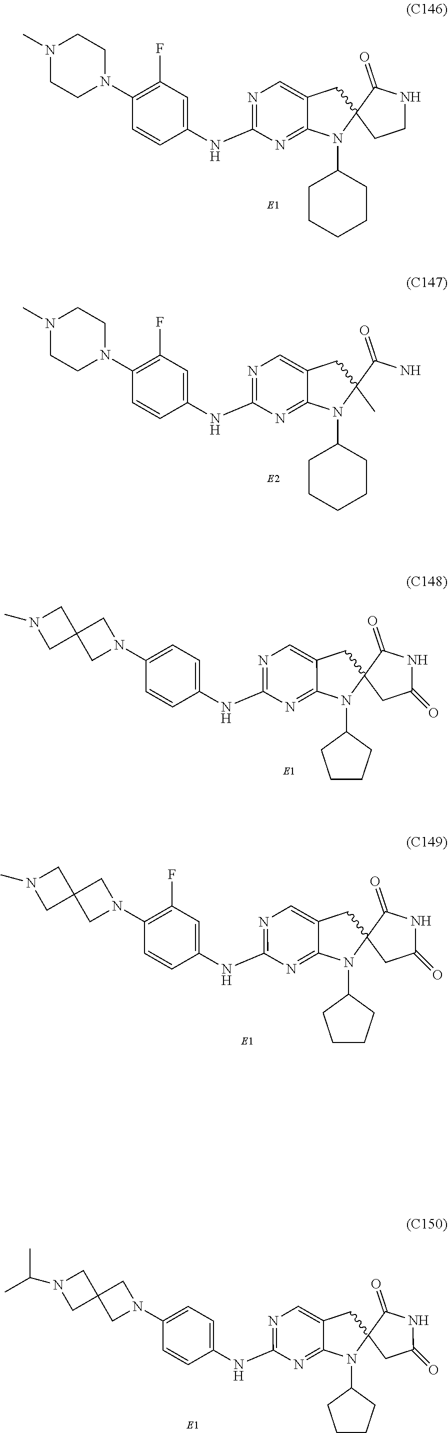 [SMILES: CN1CCN(c2ccc(Nc3ncc4c(n3)N(C3CCCCC3)C3(CCNC3=O)C4)cc2F)CC1 ; CN1CCN(c2ccc(Nc3ncc4c(n3)N(C3CCCCC3)C(C)(C(N)=O)C4)cc2F)CC1 ; CN1CC2(C1)CN(c1ccc(Nc3ncc4c(n3)N(C3CCCC3)C3(CC(=O)NC3=O)C4)cc1)C2 ; CN1CC2(C1)CN(c1ccc(Nc3ncc4c(n3)N(C3CCCC3)C3(CC(=O)NC3=O)C4)cc1F)C2 ; CC(C)N1CC2(CN(c3ccc(Nc4ncc5c(n4)N(C4CCCC4)C4(CC(=O)NC4=O)C5)cc3)C2)C1]

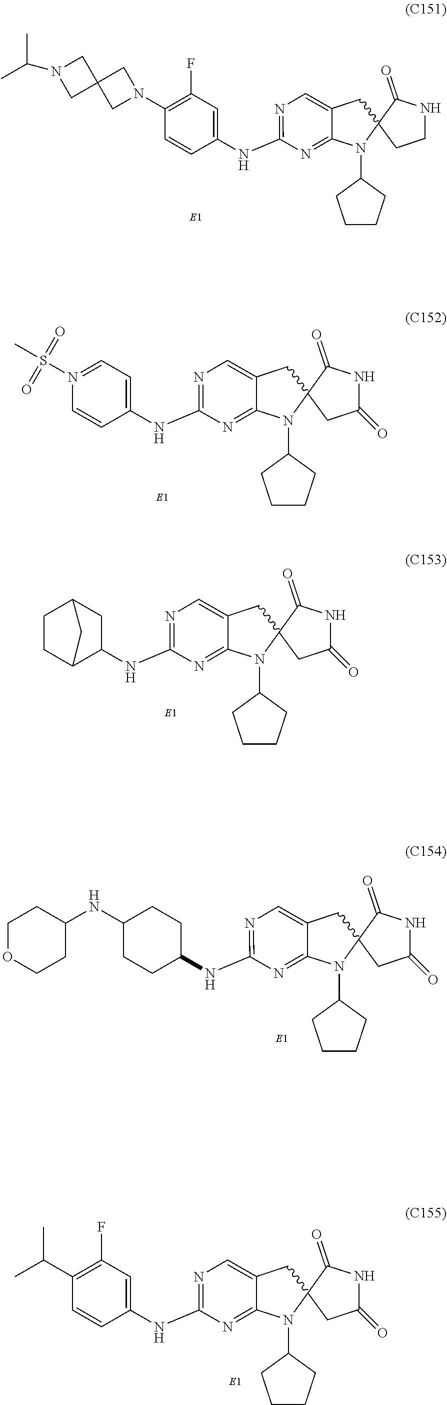

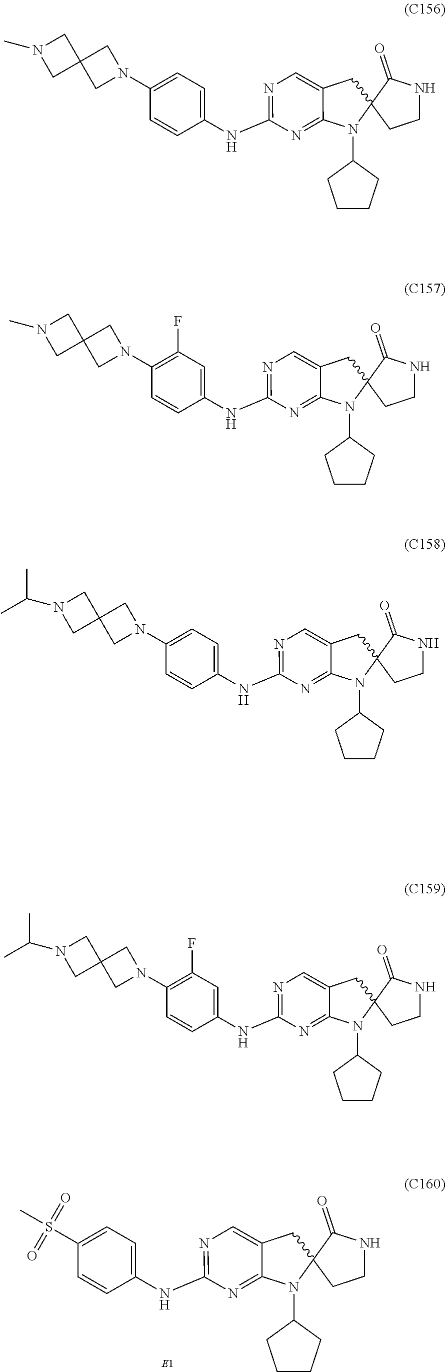 [SMILES: CN1CC2(C1)CN(c1ccc(Nc3ncc4c(n3)N(C3CCCC3)C3(CCNC3=O)C4)cc1)C2 ; CN1CC2(C1)CN(c1ccc(Nc3ncc4c(n3)N(C3CCCC3)C3(CCNC3=O)C4)cc1F)C2 ; CC(C)N1CC2(CN(c3ccc(Nc4ncc5c(n4)N(C4CCCC4)C4(CCNC4=O)C5)cc3)C2)C1 ; CC(C)N1CC2(CN(c3ccc(Nc4ncc5c(n4)N(C4CCCC4)C4(CCNC4=O)C5)cc3F)C2)C1 ; CS(=O)(=O)c1ccc(Nc2ncc3c(n2)N(C2CCCC2)C2(CCNC2=O)C3)cc1]

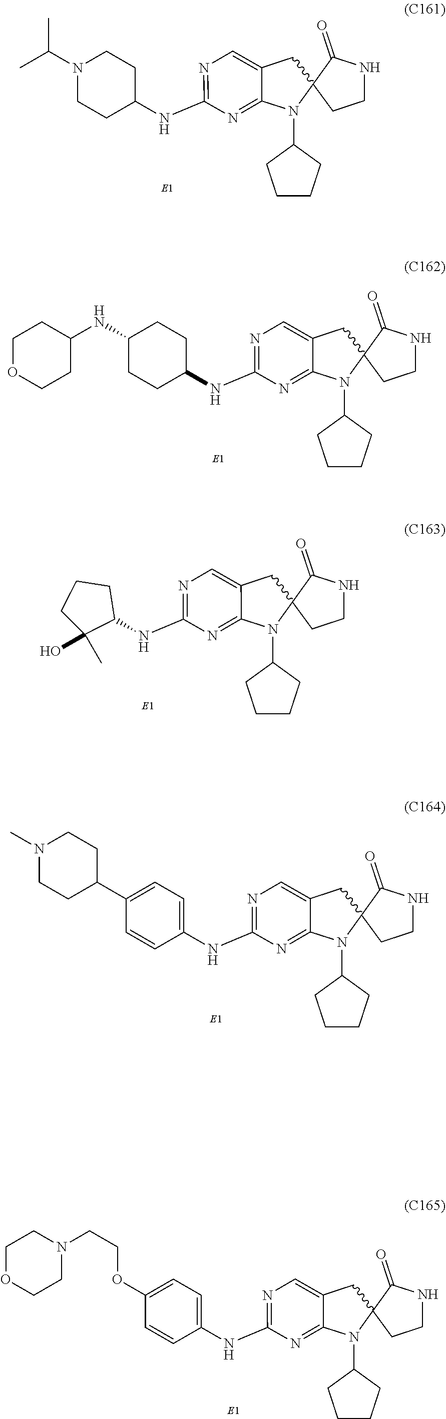

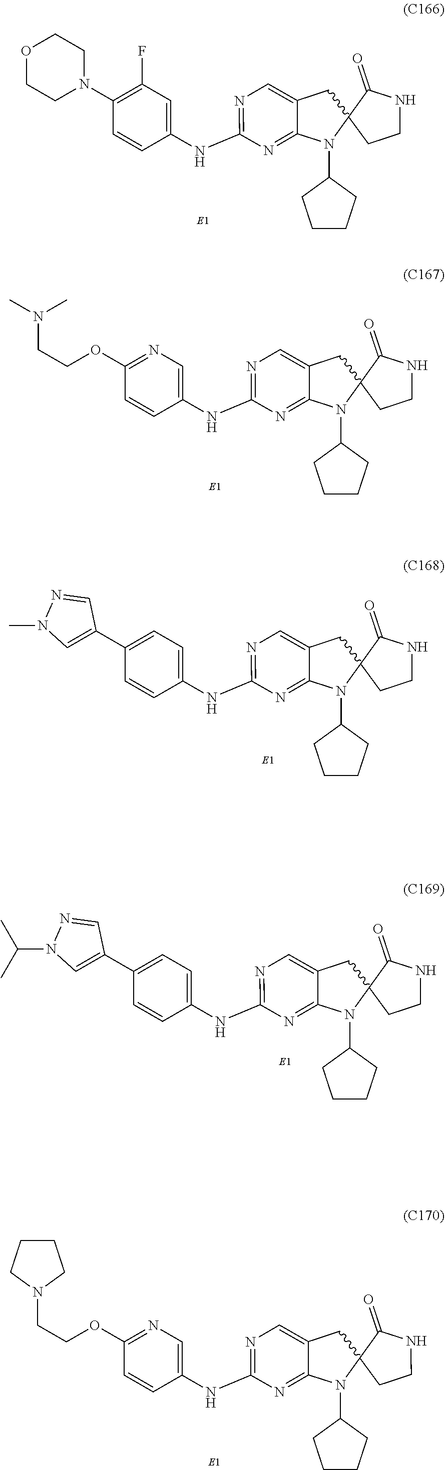 [SMILES: O=C1NCCC12Cc1cnc(Nc3ccc(N4CCOCC4)c(F)c3)nc1N2C1CCCC1 ; CN(C)CCOc1ccc(Nc2ncc3c(n2)N(C2CCCC2)C2(CCNC2=O)C3)cn1 ; Cn1cc(-c2ccc(Nc3ncc4c(n3)N(C3CCCC3)C3(CCNC3=O)C4)cc2)cn1 ; CC(C)n1cc(-c2ccc(Nc3ncc4c(n3)N(C3CCCC3)C3(CCNC3=O)C4)cc2)cn1 ; O=C1NCCC12Cc1cnc(Nc3ccc(OCCN4CCCC4)nc3)nc1N2C1CCCC1]

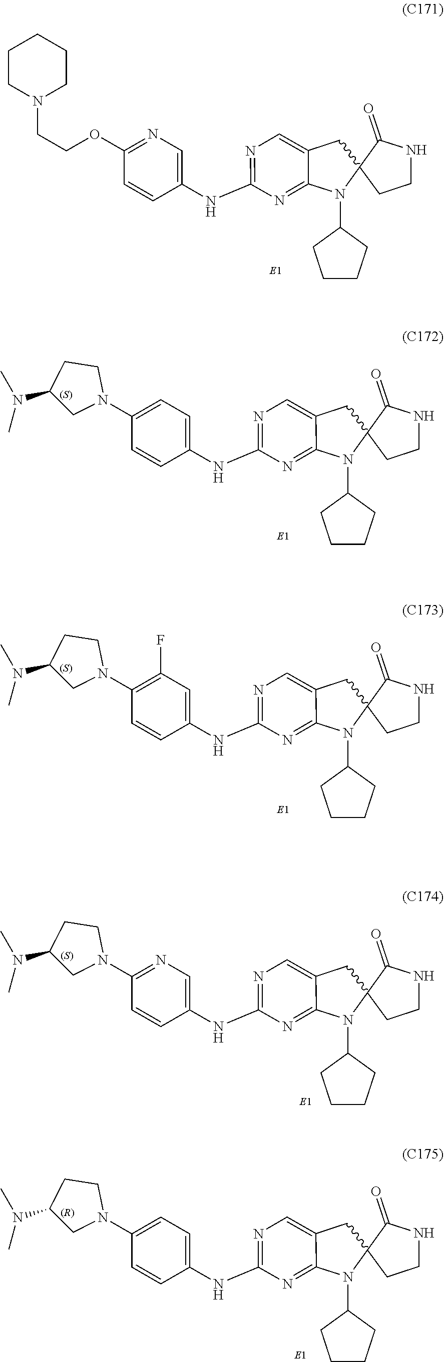

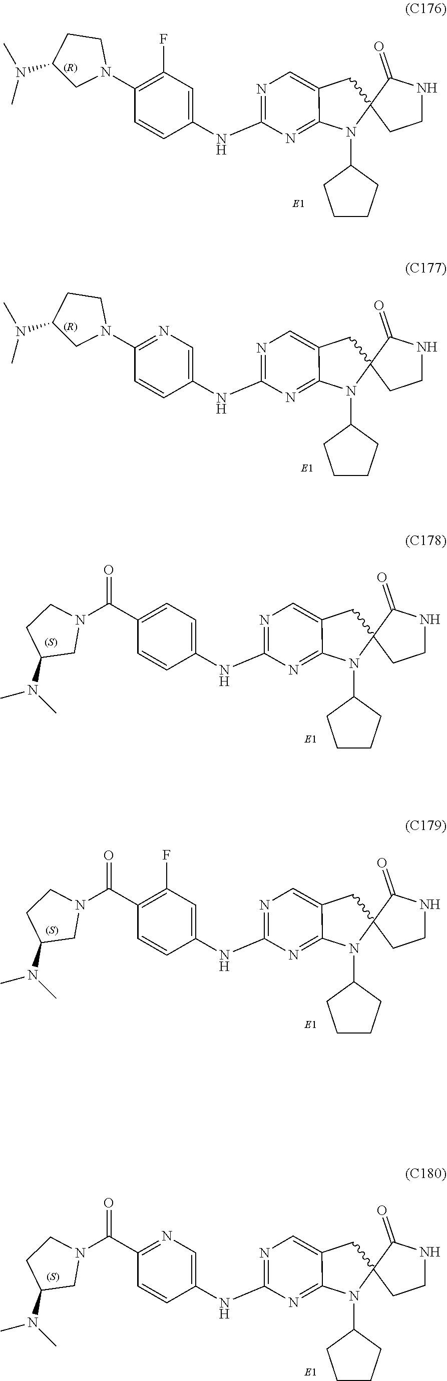

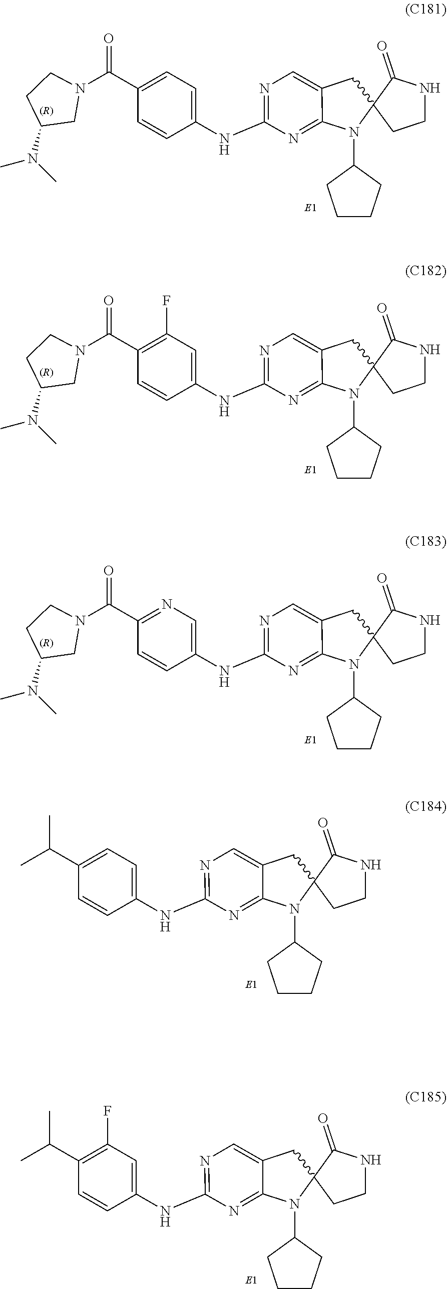

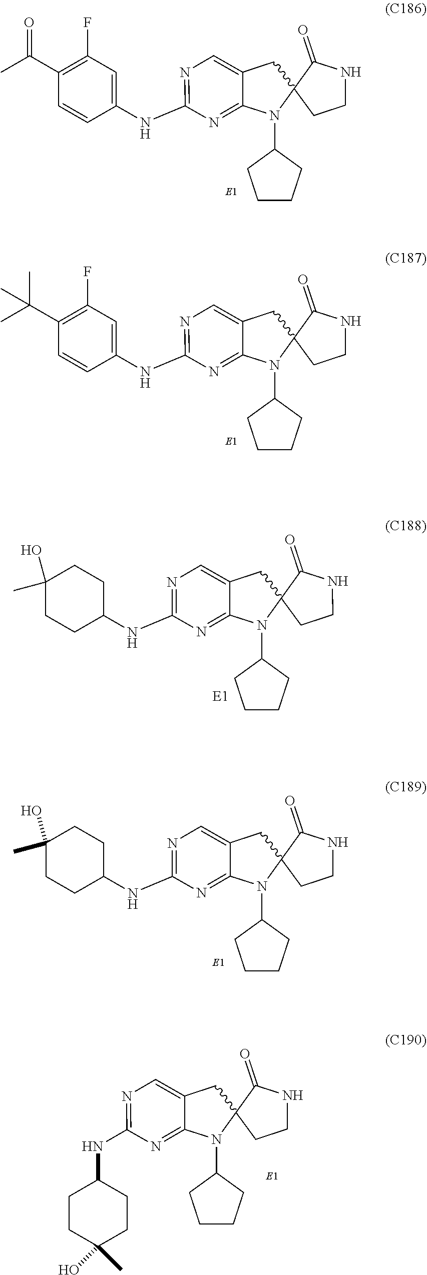

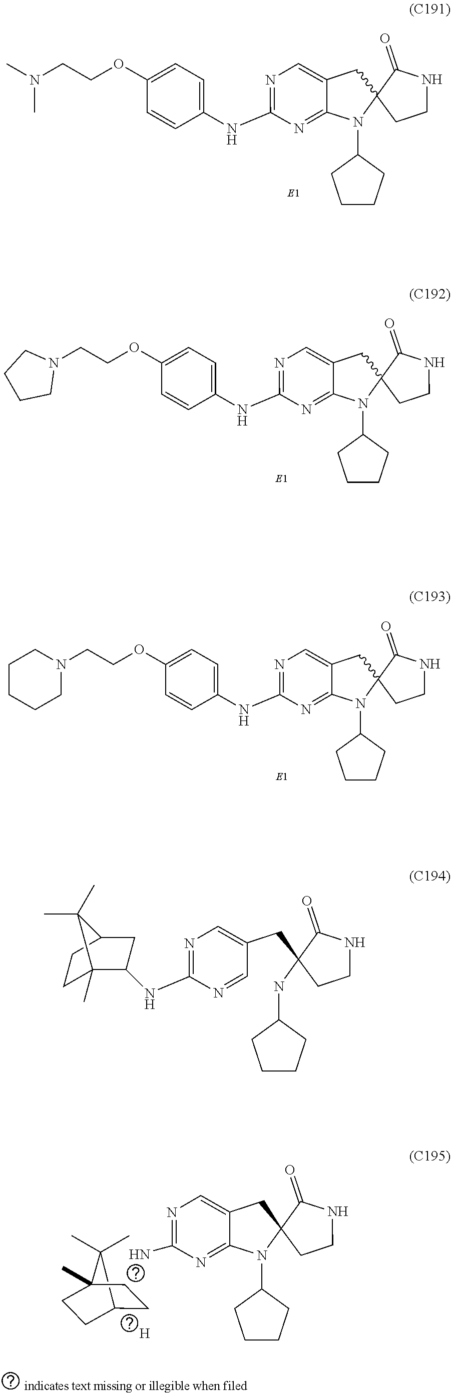

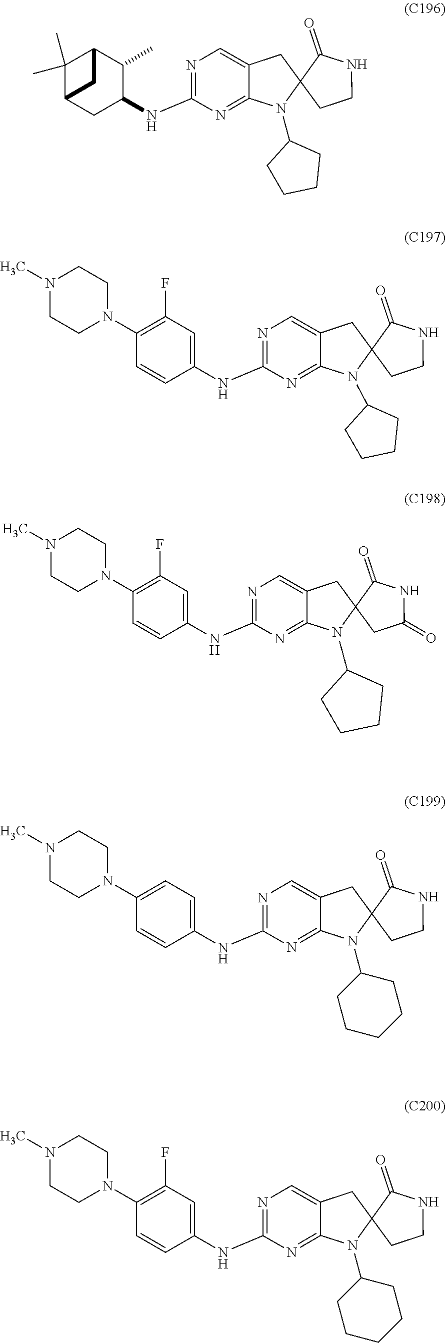

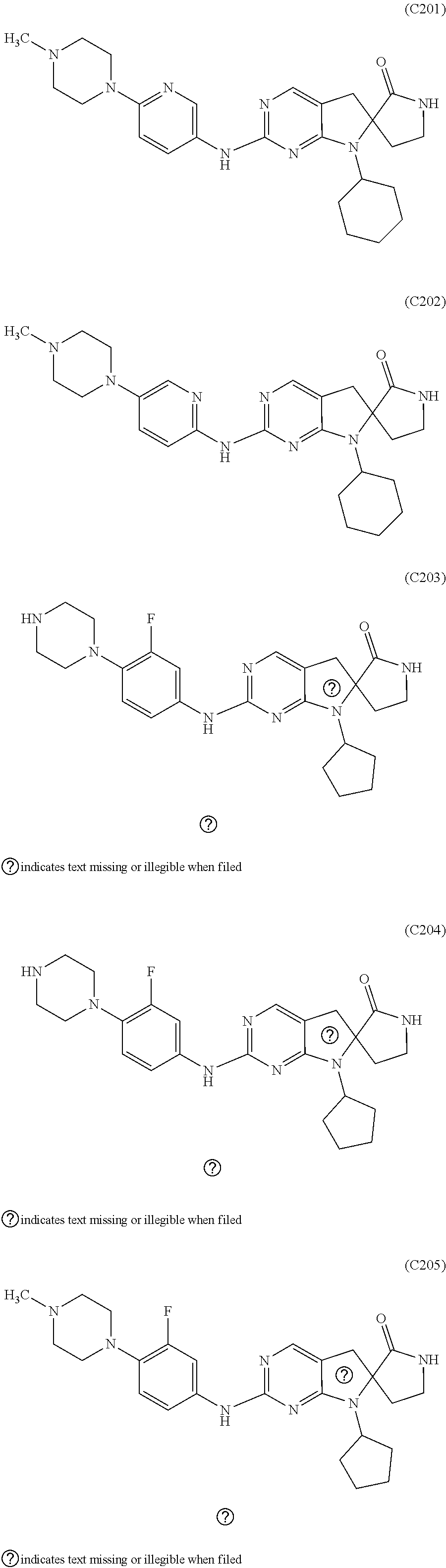 [SMILES: CN1CCN(c2ccc(Nc3ncc4c(n3)N(C3CCCCC3)C3(CCNC3=O)C4)cn2)CC1 ; CN1CCN(c2ccc(Nc3ncc4c(n3)N(C3CCCCC3)C3(CCNC3=O)C4)nc2)CC1 ; O=C1NCCC12Cc1cnc(Nc3ccc(N4CCNCC4)c(F)c3)nc1N2C1CCCC1 ; O=C1NCCC12Cc1cnc(Nc3ccc(N4CCNCC4)c(F)c3)nc1N2C1CCCC1 ; CN1CCN(c2ccc(Nc3ncc4c(n3)N(C3CCCC3)C3(CCNC3=O)C4)cc2F)CC1]

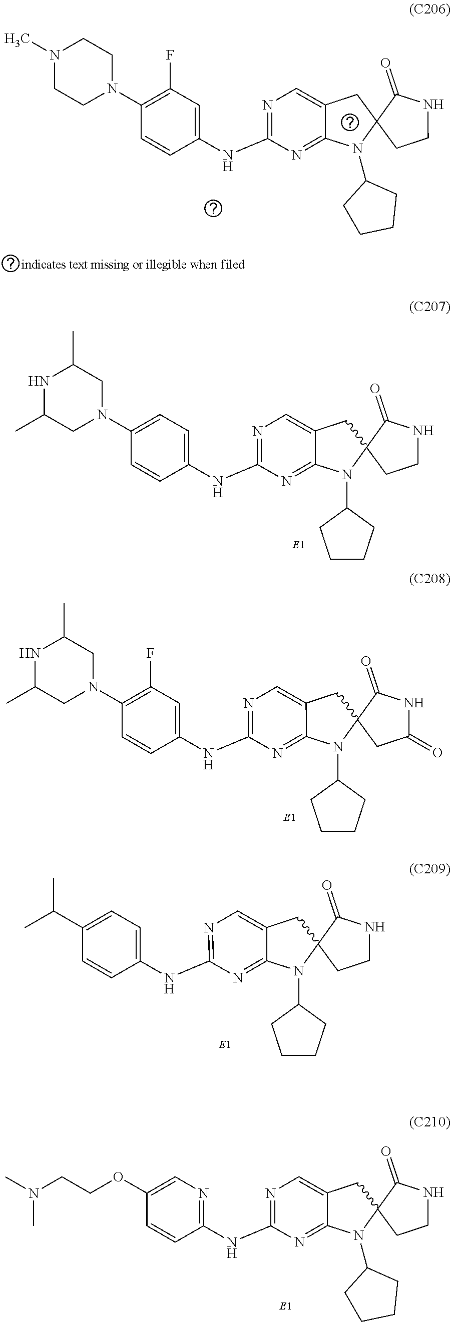 [SMILES: CN1CCN(c2ccc(Nc3ncc4c(n3)N(C3CCCC3)C3(CCNC3=O)C4)cc2F)CC1 ; CC1CN(c2ccc(Nc3ncc4c(n3)N(C3CCCC3)C3(CCNC3=O)C4)cc2)CC(C)N1 ; CC1CN(c2ccc(Nc3ncc4c(n3)N(C3CCCC3)C3(CC(=O)NC3=O)C4)cc2F)CC(C)N1 ; CC(C)c1ccc(Nc2ncc3c(n2)N(C2CCCC2)C2(CCNC2=O)C3)cc1 ; CN(C)CCOc1ccc(Nc2ncc3c(n2)N(C2CCCC2)C2(CCNC2=O)C3)nc1]

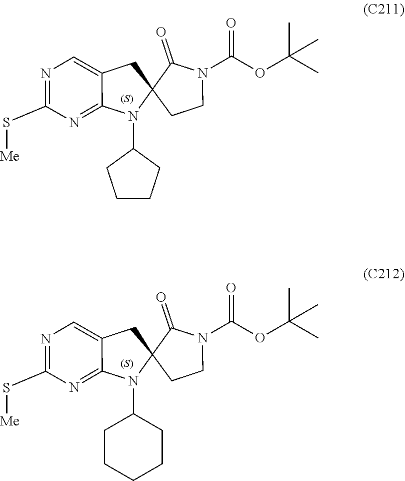

Another embodiment described herein is a pharmaceutical composition comprising one or more of Compounds 01-0212 or a pharmaceutically acceptable salt thereof and one or more pharmaceutically acceptable excipients.

Another embodiment described herein is a method of treating cancer or providing a chemotherapeutic protective effect comprising administering an effective amount of one or more of Compounds 01-0212 to a subject in need thereof.

Another embodiment described herein is a compound selected from:
7′-cyclopentyl-1-methyl-2′-((4-(4-methylpiperazin-1-yl)phenyl)amino)-5′,7′-dihydrospiro[pyrrolidine-3,6′-pyrrolo[2,3-d]pyrimidin]-2-one (C1);
7′-cyclopentyl-1-methyl-2′-((4-(piperazin-1-yl)phenyl)amino)-5′,7′-dihydrospiro[pyrrolidine-3,6′-pyrrolo[2,3-d]pyrimidin]-2-one (C2);
7′-cyclopentyl-2′-((5-(4-isopropylpiperazin-1-yl)pyridin-2-yl)amino)-1-methyl-5′,7′-dihydrospiro[pyrrolidine-3,6′-pyrrolo[2,3-d]pyrimidin]-2-one (C3);
7′-cyclopentyl-2′-((5-(2-(dimethylamino)ethoxy)pyridin-2-yl)amino)-1-methyl-5′,7′-dihydrospiro[pyrrolidine-3,6′-pyrrolo[2,3-d]pyrimidin]-2-one (C4);
7′-cyclopentyl-1-methyl-2′-((5-(2-(pyrrolidin-1-yl)ethoxy)pyridin-2-yl)amino)-5′,7′-dihydrospiro[pyrrolidine-3,6′-pyrrolo[2,3-d]pyrimidin]-2-one (C5);
7′-cyclopentyl-1-methyl-2′-((5-(2-(piperidin-1-yl)ethoxy)pyridin-2-yl)amino)-5′,7′-dihydrospiro[pyrrolidine-3,6′-pyrrolo[2,3-d]pyrimidin]-2-one (C6);
7′-cyclopentyl-1-methyl-2′-((4-(4-methylpiperazin-1-yl)phenyl)amino)-5′,7′-dihydrospiro[piperidine-3,6′-pyrrolo[2,3-d]pyrimidin]-2-one (C7);
7′-cyclopentyl-1-methyl-2′-((4-(piperazin-1-yl)phenyl)amino)-5′,7′-dihydrospiro[piperidine-3,6′-pyrrolo[2,3-d]pyrimidin]-2-one (C8);
7′-cyclopentyl-2′-((5-(4-isopropylpiperazin-1-yl)pyridin-2-yl)amino)-1-methyl-5′,7′-dihydrospiro[piperidine-3,6′-pyrrolo[2,3-d]pyrimidin]-2-one (C9);
7′-cyclopentyl-1-methyl-2′-((5-(2-(pyrrolidin-1-yl)ethoxy)pyridin-2-yl)amino)-5′,7′-dihydrospiro[piperidine-3,6′-pyrrolo[2,3-d]pyrimidin]-2-one (C10);
7′-cyclopentyl-1-methyl-2′-((5-(2-(piperidin-1-yl)ethoxy)pyridin-2-yl)amino)-5′,7′-dihydrospiro[piperidine-3,6′-pyrrolo[2,3-d]pyrimidin]-2-one (C11);
7′-cyclopentyl-1-(4-methoxybenzyl)-2′-((4-(4-methylpiperazin-1-yl)phenyl)amino)-5′,7′-dihydrospiro[pyrrolidine-3,6′-pyrrolo[2,3-d]pyrimidin]-2-one (C12);
7′-cyclopentyl-1-methyl-2′-((5-(4-methylpiperazin-1-yl)-4-(trifluoromethyl)pyridin-2-yl)amino)-5′,7′-dihydrospiro[pyrrolidine-3,6′-pyrrolo[2,3-d]pyrimidin]-2-one (C13);
7′-cyclopentyl-1-methyl-2′-((5-(piperazin-1-yl)-4-(trifluoromethyl)pyridin-2-yl)amino)-5′,7′-dihydrospiro[piperidine-3,6′-pyrrolo[2,3-d]pyrimidin]-2-one (C14);
7′-cyclopentyl-1-methyl-2′-((5-(piperazin-1-yl)-4-(trifluoromethyl)pyridin-2-yl)amino)-5′,7′-dihydrospiro[pyrrolidine-3,6′-pyrrolo[2,3-d]pyrimidin]-2-one (C15);
7′-cyclopentyl-1-(4-methoxybenzyl)-2′-((5-(piperazin-1-yl)-4-(trifluoromethyl)pyridin-2-yl)amino)-5′,7′-dihydrospiro[pyrrolidine-3,6′-pyrrolo[2,3-d]pyrimidin]-2-one (C16);
7′-cyclopentyl-2′-((5-(piperazin-1-yl)-4-(trifluoromethyl)pyridin-2-yl)amino)-5′,7′-dihydrospiro[pyrrolidine-3,6′-pyrrolo[2,3-d]pyrimidin]-2-one (C17);
7′-cyclopentyl-2′-((5-(4-methylpiperazin-1-yl)-4-(trifluoromethyl)pyridin-2-yl)amino)-5′,7′-dihydrospiro[pyrrolidine-3,6′-pyrrolo[2,3-d]pyrimidin]-2-one (C18);
7′-cyclopentyl-1-methyl-2′-((5-(piperazin-1-yl)pyridin-2-yl)amino)-5′,7′-dihydrospiro[pyrrolidine-3,6′-pyrrolo[2,3-d]pyrimidin]-2-one (C19);
7′-cyclopentyl-1-methyl-2′-((5-(4-methylpiperazin-1-yl)pyridin-2-yl)amino)-5′,7′-dihydrospiro[pyrrolidine-3,6′-pyrrolo[2,3-d]pyrimidin]-2-one (C20);
1-benzyl-7′-cyclopentyl-2′-((5-(piperazin-1-yl)pyridin-2-yl)amino)-5′,7′-dihydrospiro[pyrrolidine-3,6′-pyrrolo[2,3-d]pyrimidin]-2-one (C21);
1-benzyl-7′-cyclopentyl-2′-((5-(4-methylpiperazin-1-yl)pyridin-2-yl)amino)-5′,7′-dihydrospiro[pyrrolidine-3,6′-pyrrolo[2,3-d]pyrimidin]-2-one (C22);
1-benzyl-7′-cyclopentyl-2′-((5-(4-isopropylpiperazin-1-yl)pyridin-2-yl)amino)-5′,7′-dihydrospiro[pyrrolidine-3,6′-pyrrolo[2,3-d]pyrimidin]-2-one (C23);
1-benzyl-7′-cyclopentyl-2′-((4-(4-methylpiperazin-1-yl)phenyl)amino)-5′,7′-dihydrospiro[pyrrolidine-3,6′-pyrrolo[2,3-d]pyrimidin]-2-one (C24);
7′-cyclopentyl-1-methyl-2′-((5-(piperazin-1-yl)pyridin-2-yl)amino)-5′,7′-dihydrospiro[piperidine-3,6′-pyrrolo[2,3-d]pyrimidin]-2-one (C25);
7′-cyclopentyl-1-methyl-2′-((5-(4-methylpiperazin-1-yl)pyridin-2-yl)amino)-5′,7′-dihydrospiro[piperidine-3,6′-pyrrolo[2,3-d]pyrimidin]-2-one (C26);
1-benzyl-7′-cyclopentyl-2′-((5-(piperazin-1-yl)pyridin-2-yl)amino)-5′,7′-dihydrospiro[piperidine-3,6′-pyrrolo[2,3-d]pyrimidin]-2-one (C27);
1-benzyl-7′-cyclopentyl-2′-((5-(4-methylpiperazin-1-yl)pyridin-2-yl)amino)-5′,7′-dihydrospiro[piperidine-3,6′-pyrrolo[2,3-d]pyrimidin]-2-one (C28);
7′-cyclopentyl-1-(4-methoxybenzyl)-2′-((5-(piperazin-1-yl)pyridin-2-yl)amino)-5′,7′-dihydrospiro[pyrrolidine-3,6′-pyrrolo[2,3-d]pyrimidin]-2-one (C29);
7′-cyclopentyl-1-(4-methoxybenzyl)-2′-((5-(4-methylpiperazin-1-yl)pyridin-2-yl)amino)-5′,7′-dihydrospiro[pyrrolidine-3,6′-pyrrolo[2,3-d]pyrimidin]-2-one (C30);
7′-cyclopentyl-2′-((5-(4-isopropylpiperazin-1-yl)pyridin-2-yl)amino)-1-(4-methoxybenzyl)-5′,7′-dihydrospiro[pyrrolidine-3,6′-pyrrolo[2,3-d]pyrimidin]-2-one (C31);
7′-cyclopentyl-2′-((5-(2-(dimethylamino)ethoxy)pyridin-2-yl)amino)-1-(4-methoxybenzyl)-5′,7′-dihydrospiro[pyrrolidine-3,6′-pyrrolo[2,3-d]pyrimidin]-2-one (C32);
7′-cyclopentyl-2′-((5-(piperazin-1-yl)pyridin-2-yl)amino)-5′,7′-dihydrospiro[pyrrolidine-3,6′-pyrrolo[2,3-d]pyrimidin]-2-one (C33);
7′-cyclopentyl-2′-((5-(4-methylpiperazin-1-yl)pyridin-2-yl)amino)-5′,7′-dihydrospiro[pyrrolidine-3,6′-pyrrolo[2,3-d]pyrimidin]-2-one (C34);
7′-cyclopentyl-2′-((5-(piperazin-1-yl)pyridin-2-yl)amino)-5′,7′-dihydrospiro[piperidine-3,6′-pyrrolo[2,3-d]pyrimidin]-2-one (C35);
7′-cyclopentyl-2′-((5-(4-methylpiperazin-1-yl)pyridin-2-yl)amino)-5′,7′-dihydrospiro[piperidine-3,6′-pyrrolo[2,3-d]pyrimidin]-2-one (C36);
7′-cyclopentyl-2′-((5-(4-isopropylpiperazin-1-yl)pyridin-2-yl)amino)-5′,7′-dihydrospiro[piperidine-3,6′-pyrrolo[2,3-d]pyrimidin]-2-one (C37);
7′-cyclopentyl-2′-((5-(4-isopropylpiperazin-1-yl)pyridin-2-yl)amino)-5′,7′-dihydrospiro[pyrrolidine-3,6′-pyrrolo[2,3-d]pyrimidin]-2-one (C38);
1-benzyl-7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one (C39);
1-benzyl-7′-cyclopentyl-2′-((3-fluoro-4-(4-methylpiperazin-1-yl)phenyl)amino)-5′,7′-dihydrospiro[piperidine-3,6′-pyrrolo[2,3-d]pyrimidin]-2-one (C40);
1-benzyl-7′-cyclopentyl-2′-((2-fluoro-4-(4-methylpiperazin-1-yl)phenyl)amino)-5′,7′-dihydrospiro[pyrrolidine-3,6′-pyrrolo[2,3-d]pyrimidin]-2-one (C41);
1-benzyl-7′-cyclopentyl-2′-((2-fluoro-4-(4-methylpiperazin-1-yl)phenyl)amino)-5′,7′-dihydrospiro[piperidine-3,6′-pyrrolo[2,3-d]pyrimidin]-2-one (C42);
7′-cyclopentyl-2′-((4-(piperazin-1-yl)phenyl)amino)-5′,7′-dihydrospiro[pyrrolidine-3,6′-pyrrolo[2,3-d]pyrimidin]-2-one (C43);
7′-cyclopentyl-2′-((5-(piperazin-1-yl)-4-(trifluoromethyl)pyridin-2-yl)amino)-5′,7′-dihydrospiro[piperidine-3,6′-pyrrolo[2,3-d]pyrimidin]-2-one (C44);
7′-cyclopentyl-2′-((4-(4-methylpiperazin-1-yl)phenyl)amino)-5′,7′-dihydrospiro[pyrrolidine-3,6′-pyrrolo[2,3-d]pyrimidin]-2-one (C45);
7′-cyclopentyl-2′-((4-(4-isopropylpiperazin-1-yl)phenyl)amino)-5′,7′-dihydrospiro[pyrrolidine-3,6′-pyrrolo[2,3-d]pyrimidin]-2-one (C46);
7′-cyclopentyl-2′-((3-fluoro-4-(piperazin-1-yl)phenyl)amino)-5′,7′-dihydrospiro[pyrrolidine-3,6′-pyrrolo[2,3-d]pyrimidin]-2-one (C47);
7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one (C48);
7′-cyclopentyl-2′-((3-fluoro-4-(4-isopropylpiperazin-1-yl)phenyl)amino)-5′,7′-dihydrospiro[pyrrolidine-3,6′-pyrrolo[2,3-d]pyrimidin]-2-one (C49);
7′-cyclopentyl-2′-((2-fluoro-4-(piperazin-1-yl)phenyl)amino)-5′,7′-dihydrospiro[pyrrolidine-3,6′-pyrrolo[2,3-d]pyrimidin]-2-one (C50);
7′-cyclopentyl-2′-((2-fluoro-4-(4-methylpiperazin-1-yl)phenyl)amino)-5′,7′-dihydrospiro[pyrrolidine-3,6′-pyrrolo[2,3-d]pyrimidin]-2-one (C51);
7′-cyclopentyl-2′-((2-fluoro-4-(4-isopropylpiperazin-1-yl)phenyl)amino)-5′,7′-dihydrospiro[pyrrolidine-3,6′-pyrrolo[2,3-d]pyrimidin]-2-one (C52);
7′-cyclopentyl-2′-((6-(piperazin-1-yl)pyridin-3-yl)amino)-5′,7′-dihydrospiro[pyrrolidine-3,6′-pyrrolo[2,3-d]pyrimidin]-2-one (C53);
7′-cyclopentyl-2′-((6-(4-methylpiperazin-1-yl)pyridin-3-yl)amino)-5′,7′-dihydrospiro[pyrrolidine-3,6′-pyrrolo[2,3-d]pyrimidin]-2-one (C54);
7′-cyclopentyl-2′-((6-(4-isopropylpiperazin-1-yl)pyridin-3-yl)amino)-5′,7′-dihydrospiro[pyrrolidine-3,6′-pyrrolo[2,3-d]pyrimidin]-2-one (C55);
7′-cyclopentyl-2′-((1′,2′,3′,6′-tetrahydro-[3,4′-bipyridin]-6-yl)amino)-5′,7′-dihydrospiro[pyrrolidine-3,6′-pyrrolo[2,3-d]pyrimidin]-2-one (C56);
7′-cyclopentyl-2′-((1′-isopropyl-1′,2′,3′,6′-tetrahydro-[3,4′-bipyridin]-6-yl)amino)-5′,7′-dihydrospiro[pyrrolidine-3,6′-pyrrolo[2,3-d]pyrimidin]-2-one (C57);
7′-cyclopentyl-2′-((3-fluoro-4-morpholinophenyl)amino)-5′,7′-dihydrospiro[pyrrolidine-3,6′-pyrrolo[2,3-d]pyrimidin]-2-one (C58);
7′-cyclopentyl-2′-((4-(2-(dimethylamino)ethoxy)phenyl)amino)-5′,7′-dihydrospiro[pyrrolidine-3,6′-pyrrolo[2,3-d]pyrimidin]-2-one (C59);
7′-cyclopentyl-2′-((5-(2-(dimethylamino)ethoxy)pyridin-2-yl)amino)-5′,7′-dihydrospiro[pyrrolidine-3,6′-pyrrolo[2,3-d]pyrimidin]-2-one (C60);
7′-cyclopentyl-2′-((6-(2-(dimethylamino)ethoxy)pyridin-3-yl)amino)-5′,7′-dihydrospiro[pyrrolidine-3,6′-pyrrolo[2,3-d]pyrimidin]-2-one (C61);
7′-cyclopentyl-2′-((4-(4-isopropylpiperazin-1-yl)-3-methoxyphenyl)amino)-5′,7′-dihydrospiro[pyrrolidine-3,6′-pyrrolo[2,3-d]pyrimidin]-2-one (C62);
7′-cyclopentyl-2′-((4-(4-isopropylpiperazin-1-yl)-3-methylphenyl)amino)-5′,7′-dihydrospiro[pyrrolidine-3,6′-pyrrolo[2,3-d]pyrimidin]-2-one (C63); 2-((5-(4-cyanopiperazin-1-yl)pyridin-2-yl)amino)-7-cyclopentyl-N,N-dimethyl-7H-pyrrolo[2,3-d]pyrimidine-6-carboxamide (C64);
7′-cyclopentyl-1-methyl-2′-((5-(piperazin-1-yl)pyridin-2-yl)amino)-5′,7′-dihydrospiro[pyrrolidine-3,6′-pyrrolo[2,3-d]pyrimidine]-2,5-dione (C65);
7′-cyclopentyl-1-methyl-2′-((5-(4-methylpiperazin-1-yl)pyridin-2-yl)amino)-5′,7′-dihydrospiro[pyrrolidine-3,6′-pyrrolo[2,3-d]pyrimidine]-2,5-dione (C66);
7′-cyclopentyl-2′-((5-(4-isopropylpiperazin-1-yl)pyridin-2-yl)amino)-1-methyl-5′,7′-dihydrospiro[pyrrolidine-3,6′-pyrrolo[2,3-d]pyrimidine]-2,5-dione (C67);
7′-cyclopentyl-1-methyl-2′-((4-(piperazin-1-yl)phenyl)amino)-5′,7′-dihydrospiro[pyrrolidine-3,6′-pyrrolo[2,3-d]pyrimidine]-2,5-dione (C68);
7′-cyclopentyl-1-methyl-2′-((4-(4-methylpiperazin-1-yl)phenyl)amino)-5′,7′-dihydrospiro[pyrrolidine-3,6′-pyrrolo[2,3-d]pyrimidine]-2,5-dione (C69);
7′-cyclopentyl-2′-((4-(4-isopropylpiperazin-1-yl)phenyl)amino)-1-methyl-5′,7′-dihydrospiro[pyrrolidine-3,6′-pyrrolo[2,3-d]pyrimidine]-2,5-dione (C70);
7′-cyclopentyl-2′-((3-fluoro-4-(piperazin-1-yl)phenyl)amino)-1-methyl-5′,7′-dihydrospiro[pyrrolidine-3,6′-pyrrolo[2,3-d]pyrimidine]-2,5-dione (C72);
7′-cyclopentyl-2′-((3-fluoro-4-(4-methylpiperazin-1-yl)phenyl)amino)-1-methyl-5′,7′-dihydrospiro[pyrrolidine-3,6′-pyrrolo[2,3-d]pyrimidine]-2,5-dione (C72);
7′-cyclopentyl-2′-((3-fluoro-4-(4-isopropylpiperazin-1-yl)phenyl)amino)-1-methyl-5′,7′-dihydrospiro[pyrrolidine-3,6′-pyrrolo[2,3-d]pyrimidine]-2,5-dione (C73);
7′-cyclopentyl-2′-((5-(piperazin-1-yl)pyridin-2-yl)amino)-5′,7′-dihydrospiro[pyrrolidine-3,6′-pyrrolo[2,3-d]pyrimidine]-2,5-dione (C74);
7′-cyclopentyl-2′-((5-(4-methylpiperazin-1-yl)pyridin-2-yl)amino)-5′,7′-dihydrospiro[pyrrolidine-3,6′-pyrrolo[2,3-d]pyrimidine]-2,5-dione (C75);
7′-cyclopentyl-2′-((5-(4-isopropylpiperazin-1-yl)pyridin-2-yl)amino)-5′,7′-dihydrospiro[pyrrolidine-3,6′-pyrrolo[2,3-d]pyrimidine]-2,5-dione (C76);
7′-cyclopentyl-2′-((4-(piperazin-1-yl)phenyl)amino)-5′,7′-dihydrospiro[pyrrolidine-3,6′-pyrrolo[2,3-d]pyrimidine]-2,5-dione (C77);
7′-cyclopentyl-2′-((4-(4-methylpiperazin-1-yl)phenyl)amino)-5′,7′-dihydrospiro[pyrrolidine-3,6′-pyrrolo[2,3-d]pyrimidine]-2,5-dione (C78);
7′-cyclopentyl-2′-((4-(4-isopropylpiperazin-1-yl)phenyl)amino)-5′,7′-dihydrospiro[pyrrolidine-3,6′-pyrrolo[2,3-d]pyrimidine]-2,5-dione (C79);
7′-cyclopentyl-2′-((3-fluoro-4-(piperazin-1-yl)phenyl)amino)-5′,7′-dihydrospiro[pyrrolidine-3,6′-pyrrolo[2,3-d]pyrimidine]-2,5-dione (C80);
7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidine]-2,5-dione (C81);
7′-cyclopentyl-2′-((3-fluoro-4-(4-isopropylpiperazin-1-yl)phenyl)amino)-5′,7′-dihydrospiro[pyrrolidine-3,6′-pyrrolo[2,3-d]pyrimidine]-2,5-dione (C82);
7′-cyclopentyl-2′-((6-(piperazin-1-yl)pyridin-3-yl)amino)-5′,7′-dihydrospiro[pyrrolidine-3,6′-pyrrolo[2,3-d]pyrimidine]-2,5-dione (C83);
7′-cyclopentyl-2′-((6-(4-methylpiperazin-1-yl)pyridin-3-yl)amino)-5′,7′-dihydrospiro[pyrrolidine-3,6′-pyrrolo[2,3-d]pyrimidine]-2,5-dione (C84);
7′-cyclopentyl-2′-((6-(4-isopropylpiperazin-1-yl)pyridin-3-yl)amino)-5′,7′-dihydrospiro[pyrrolidine-3,6′-pyrrolo[2,3-d]pyrimidine]-2,5-dione (C85);
7′-cyclopentyl-2′-((3-fluoro-4-morpholinophenyl)amino)-5′,7′-dihydrospiro[pyrrolidine-3,6′-pyrrolo[2,3-d]pyrimidine]-2,5-dione (C86);
7′-cyclopentyl-2′-((2-fluoro-4-(4-methylpiperazin-1-yl)phenyl)amino)-5′,7′-dihydrospiro[pyrrolidine-3,6′-pyrrolo[2,3-d]pyrimidine]-2,5-dione (C87);
7′-cyclopentyl-2′-((6-(2-(dimethylamino)ethoxy)pyridin-3-yl)amino)-5′,7′-dihydrospiro[pyrrolidine-3,6′-pyrrolo[2,3-d]pyrimidine]-2,5-dione (C88);
7′-cyclopentyl-2′-((4-(2-(dimethylamino)ethoxy)phenyl)amino)-5′,7′-dihydrospiro[pyrrolidine-3,6′-pyrrolo[2,3-d]pyrimidine]-2,5-dione (C89);
7′-cyclopentyl-2′-((6-(2-(pyrrolidin-1-yl)ethoxy)pyridin-3-yl)amino)-5′,7′-dihydrospiro[pyrrolidine-3,6′-pyrrolo[2,3-d]pyrimidine]-2,5-dione (C90);
7′-cyclopentyl-2′-((3-fluoro-4-(2-(pyrrolidin-1-yl)ethoxy)phenyl)amino)-5′,7′-dihydrospiro[pyrrolidine-3,6′-pyrrolo[2,3-d]pyrimidine]-2,5-dione (C91);
7′-cyclopentyl-2′-((5-(2-(dimethylamino)ethoxy)pyridin-2-yl)amino)-5′,7′-dihydrospiro[pyrrolidine-3,6′-pyrrolo[2,3-d]pyrimidine]-2,5-dione (C92);
7′-cyclopentyl-2′-((1′,2′,3′,6′-tetrahydro-[3,4′-bipyridin]-6-yl)amino)-5′,7′-dihydrospiro[pyrrolidine-3,6′-pyrrolo[2,3-d]pyrimidine]-2,5-dione (C93);
7′-cyclopentyl-2′-((1′-isopropyl-1′,2′,3′,6′-tetrahydro-[3,4′-bipyridin]-6-yl)amino)-5′,7′-dihydrospiro[pyrrolidine-3,6′-pyrrolo[2,3-d]pyrimidine]-2,5-dione (C94);
7′-cyclopentyl-2′-((4-(4-morpholinopiperidin-1-yl)phenyl)amino)-5′,7′-dihydrospiro[pyrrolidine-3,6′-pyrrolo[2,3-d]pyrimidin]-2-one (C95);
7′-cyclopentyl-2′-((6-(4-morpholinopiperidin-1-yl)pyridin-3-yl)amino)-5′,7′-dihydrospiro[pyrrolidine-3,6′-pyrrolo[2,3-d]pyrimidin]-2-one (C96);
7′-cyclopentyl-2′-((3-fluoro-4-(4-morpholinopiperidin-1-yl)phenyl)amino)-5′,7′-dihydrospiro[pyrrolidine-3,6′-pyrrolo[2,3-d]pyrimidin]-2-one (C97);
7′-cyclopentyl-2′-((5-(4-methylpiperazin-1-yl)pyridin-2-yl)amino)-6′,7′-dihydrospiro[pyrrolidine-3,5′-pyrrolo[2,3-d]pyrimidin]-2-one (C98);
7′-cyclopentyl-2′-((5-(piperazin-1-yl)pyridin-2-yl)amino)spiro[pyrrolidine-3,5′-pyrrolo[2,3-d]pyrimidine]-2,6′(7′H)-dione (C99);
7′-cyclopentyl-2′-((5-(4-methylpiperazin-1-yl)pyridin-2-yl)amino)spiro[pyrrolidine-3,5′-pyrrolo[2,3-d]pyrimidine]-2,6′(7′H)-dione (C100);
7′-cyclopentyl-2′-((4-(4-methylpiperazin-1-yl)phenyl)amino)spiro[pyrrolidine-3,5′-pyrrolo[2,3-d]pyrimidine]-2,6′(7′H)-dione (C101);
7′-cyclopentyl-2′-((3-fluoro-4-(piperazin-1-yl)phenyl)amino)spiro[pyrrolidine-3,5′-pyrrolo[2,3-d]pyrimidine]-2,6′(7′H)-dione (C102);
7′-cyclopentyl-2′-((3-fluoro-4-(4-methylpiperazin-1-yl)phenyl)amino)spiro[pyrrolidine-3,5′-pyrrolo[2,3-d]pyrimidine]-2,6′(7′H)-dione (C103);
7′-cyclopentyl-2′-((3-fluoro-4-(4-isopropylpiperazin-1-yl)phenyl)amino)spiro[pyrrolidine-3,5′-pyrrolo[2,3-d]pyrimidine]-2,6′(7′H)-dione (C104);
7′-cyclopentyl-2′-((4-((S)-3-(dimethylamino)pyrrolidin-1-yl)-3-fluorophenyl)amino)spiro[pyrrolidine-3,5′-pyrrolo[2,3-d]pyrimidine]-2,6′(7′H)-dione (C105);
7′-cyclopentyl-2′-((3-fluoro-4-(4-morpholinopiperidin-1-yl)phenyl)amino)spiro[pyrrolidine-3,5′-pyrrolo[2,3-d]pyrimidine]-2,6′(7′H)-dione (C106);
7′-cyclopentyl-2′-((3-fluoro-4-(2,6-diazaspiro[3.3]heptan-2-yl)phenyl)amino)-5′,7′-dihydrospiro[pyrrolidine-3,6′-pyrrolo[2,3-d]pyrimidin]-2-one (C107);
7′-cyclopentyl-2′-((4-(2-(dimethylamino)ethoxy)phenyl)amino)-5′,7′-dihydrospiro[pyrrolidine-3,6′-pyrrolo[2,3-d]pyrimidin]-2-one (C108);
7′-cyclohexyl-2′-((4-(2-(dimethylamino)ethoxy)phenyl)amino)-5′,7′-dihydrospiro[pyrrolidine-3,6′-pyrrolo[2,3-d]pyrimidin]-2-one (C109);
7′-cyclopentyl-2′-((6-(2-(dimethylamino)ethoxy)pyridin-3-yl)amino)-5′,7′-dihydrospiro[pyrrolidine-3,6′-pyrrolo[2,3-d]pyrimidin]-2-one (C110);
7′-cyclopentyl-2′-((4-(2-(dimethylamino)ethoxy)-3-fluorophenyl)amino)-5′,7′-dihydrospiro[pyrrolidine-3,6′-pyrrolo[2,3-d]pyrimidin]-2-one (C111);
7′-cyclopentyl-2′-((4-(2-(dimethylamino)ethoxy)-3-methylphenyl)amino)-5′,7′-dihydrospiro[pyrrolidine-3,6′-pyrrolo[2,3-d]pyrimidin]-2-one (C112);
7′-cyclopentyl-2′-((4-(2-(dimethylamino)ethoxy)-3-methoxyphenyl)amino)-5′,7′-dihydrospiro[pyrrolidine-3,6′-pyrrolo[2,3-d]pyrimidin]-2-one (C113);
7′-cyclopentyl-2′-((4-(piperazin-1-yl)phenyl)amino)-5′,7′-dihydrospiro[pyrrolidine-3,6′-pyrrolo[2,3-d]pyrimidin]-2-one (C114);
7′-cyclopentyl-2′-((4-(4-methylpiperazin-1-yl)phenyl)amino)-5′,7′-dihydrospiro[pyrrolidine-3,6′-pyrrolo[2,3-d]pyrimidin]-2-one (C115);
7′-cyclopentyl-2′-((4-(4-isopropylpiperazin-1-yl)phenyl)amino)-5′,7′-dihydrospiro[pyrrolidine-3,6′-pyrrolo[2,3-d]pyrimidin]-2-one (C116);
7′-cyclopentyl-2′-((3-fluoro-4-(piperazin-1-yl)phenyl)amino)-5′,7′-dihydrospiro[pyrrolidine-3,6′-pyrrolo[2,3-d]pyrimidin]-2-one (C117);
7′-cyclopentyl-2′-((3-fluoro-4-(4-isopropylpiperazin-1-yl)phenyl)amino)-5′,7′-dihydrospiro[pyrrolidine-3,6′-pyrrolo[2,3-d]pyrimidin]-2-one (C118);
7′-cyclopentyl-2′-((3-methyl-4-(piperazin-1-yl)phenyl)amino)-5′,7′-dihydrospiro[pyrrolidine-3,6′-pyrrolo[2,3-d]pyrimidin]-2-one (C119);
7′-cyclopentyl-2′-((3-methyl-4-(4-methylpiperazin-1-yl)phenyl)amino)-5′,7′-dihydrospiro[pyrrolidine-3,6′-pyrrolo[2,3-d]pyrimidin]-2-one (C120);
7′-cyclopentyl-2′-((4-(4-isopropylpiperazin-1-yl)-3-methylphenyl)amino)-5′,7′-dihydrospiro[pyrrolidine-3,6′-pyrrolo[2,3-d]pyrimidin]-2-one (C121);
7′-cyclopentyl-2′-((3-methoxy-4-(piperazin-1-yl)phenyl)amino)-5′,7′-dihydrospiro[pyrrolidine-3,6′-pyrrolo[2,3-d]pyrimidin]-2-one (C122);
7′-cyclopentyl-2′-((3-methoxy-4-(4-methylpiperazin-1-yl)phenyl)amino)-5′,7′-dihydrospiro[pyrrolidine-3,6′-pyrrolo[2,3-d]pyrimidin]-2-one (C123);
7′-cyclopentyl-2′-((4-(4-isopropylpiperazin-1-yl)-3-methoxyphenyl)amino)-5′,7′-dihydrospiro[pyrrolidine-3,6′-pyrrolo[2,3-d]pyrimidin]-2-one (C124);
7′-cyclopentyl-2′-((3-fluoro-4-(2,6-diazaspiro[3.3]heptan-2-yl)phenyl)amino)-5′,7′-dihydrospiro[pyrrolidine-3,6′-pyrrolo[2,3-d]pyrimidin]-2-one (C125);
2′-((4-(2,6-diazaspiro[3.3]heptan-2-yl)phenyl)amino)-7′-cyclopentyl-5′,7′-dihydrospiro[pyrrolidine-3,6′-pyrrolo[2,3-d]pyrimidin]-2-one (C126);
2′-((6-(2,6-diazaspiro[3.3]heptan-2-yl)pyridin-3-yl)amino)-7′-cyclopentyl-5′,7′-dihydrospiro[pyrrolidine-3,6′-pyrrolo[2,3-d]pyrimidin]-2-one (C127);
7′-cyclopentyl-2′-((6-(piperazin-1-yl)pyridin-3-yl)amino)-5′,7′-dihydrospiro[pyrrolidine-3,6′-pyrrolo[2,3-d]pyrimidin]-2-one (C128);
7′-cyclopentyl-2′-((6-(4-methylpiperazin-1-yl)pyridin-3-yl)amino)-5′,7′-dihydrospiro[pyrrolidine-3,6′-pyrrolo[2,3-d]pyrimidin]-2-one (C129);
7′-cyclopentyl-2′-((6-(4-isopropylpiperazin-1-yl)pyridin-3-yl)amino)-5′,7′-dihydrospiro[pyrrolidine-3,6′-pyrrolo[2,3-d]pyrimidin]-2-one (C130);
7′-cyclopentyl-2′-((4-(4-morpholinopiperidin-1-yl)phenyl)amino)-5′,7′-dihydrospiro[pyrrolidine-3,6′-pyrrolo[2,3-d]pyrimidin]-2-one (C131);
7′-cyclopentyl-2′-((3-fluoro-4-(4-morpholinopiperidin-1-yl)phenyl)amino)-5′,7′-dihydrospiro[pyrrolidine-3,6′-pyrrolo[2,3-d]pyrimidin]-2-one (C132);
7′-cyclopentyl-2′-((6-(4-morpholinopiperidin-1-yl)pyridin-3-yl)amino)-5′,7′-dihydrospiro[pyrrolidine-3,6′-pyrrolo[2,3-d]pyrimidin]-2-one (C133);
7′-cyclopentyl-2′-((4-(4-isopropylpiperazin-1-yl)phenyl)amino)-5′,7′-dihydrospiro[pyrrolidine-3,6′-pyrrolo[2,3-d]pyrimidine]-2,5-dione (C134);
7′-cyclopentyl-2′-((4-(4-isopropylpiperazin-1-yl)phenyl)amino)-5′,7′-dihydrospiro[pyrrolidine-3,6′-pyrrolo[2,3-d]pyrimidine]-2,5-dione (C135);
7′-cyclopentyl-2′-((4-(4-methylpiperazin-1-yl)phenyl)amino)-5′,7′-dihydrospiro[pyrrolidine-3,6′-pyrrolo[2,3-d]pyrimidine]-2,5-dione (C136);
7′-cyclopentyl-2′-((4-(4-methylpiperazin-1-yl)phenyl)amino)-5′,7′-dihydrospiro[pyrrolidine-3,6′-pyrrolo[2,3-d]pyrimidine]-2,5-dione (C137);
7′-cyclopentyl-2′-((4-(piperazin-1-yl)phenyl)amino)-5′,7′-dihydrospiro[pyrrolidine-3,6′-pyrrolo[2,3-d]pyrimidine]-2,5-dione (C138);
7′-cyclopentyl-2′-((4-(piperazin-1-yl)phenyl)amino)-5′,7′-dihydrospiro[pyrrolidine-3,6′-pyrrolo[2,3-d]pyrimidine]-2,5-dione (C139);
7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidine]-2,5-dione (C140);
7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidine]-2,5-dione (C141);
7′-cyclohexyl-2′-((4-(piperazin-1-yl)phenyl)amino)-5′,7′-dihydrospiro[pyrrolidine-3,6′-pyrrolo[2,3-d]pyrimidin]-2-one (C142);
7′-cyclohexyl-2′-((4-(piperazin-1-yl)phenyl)amino)-5′,7′-dihydrospiro[pyrrolidine-3,6′-pyrrolo[2,3-d]pyrimidin]-2-one (C143);
7′-cyclohexyl-2′-((4-(4-methylpiperazin-1-yl)phenyl)amino)-5′,7′-dihydrospiro[pyrrolidine-3,6′-pyrrolo[2,3-d]pyrimidin]-2-one (C144);
7′-cyclohexyl-2′-((4-(4-methylpiperazin-1-yl)phenyl)amino)-5′,7′-dihydrospiro[pyrrolidine-3,6′-pyrrolo[2,3-d]pyrimidin]-2-one (C145);
7′-cyclohexyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one (C146);
7′-cyclohexyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one (C147);
7′-cyclopentyl-2′-((4-(6-methyl-2,6-diazaspiro[3.3]heptan-2-yl)phenyl)amino)-5′,7′-dihydrospiro[pyrrolidine-3,6′-pyrrolo[2,3-d]pyrimidine]-2,5-dione (C148);
7′-cyclopentyl-2′-((3-fluoro-4-(6-methyl-2,6-diazaspiro[3.3]heptan-2-yl)phenyl)amino)-5′,7′-dihydrospiro[pyrrolidine-3,6′-pyrrolo[2,3-d]pyrimidine]-2,5-dione (C149);
7′-cyclopentyl-2′-((4-(6-isopropyl-2,6-diazaspiro[3.3]heptan-2-yl)phenyl)amino)-5′,7′-dihydrospiro[pyrrolidine-3,6′-pyrrolo[2,3-d]pyrimidine]-2,5-dione (C150);
7′-cyclopentyl-2′-((3-fluoro-4-(6-isopropyl-2,6-diazaspiro[3.3]heptan-2-yl)phenyl)amino)-5′,7′-dihydrospiro[pyrrolidine-3,6′-pyrrolo[2,3-d]pyrimidine]-2,5-dione (C151);
7′-cyclopentyl-2′-((1-(methylsulfonyl)piperidin-4-yl)amino)-5′,7′-dihydrospiro[pyrrolidine-3,6′-pyrrolo[2,3-d]pyrimidine]-2,5-dione (C152);
2′-(bicyclo[2.2.1]heptan-2-ylamino)-7′-cyclopentyl-5′,7′-dihydrospiro[pyrrolidine-3,6′-pyrrolo[2,3-d]pyrimidine]-2,5-dione (C153);
7′-cyclopentyl-2′-(((1 r,4r)-4-((tetrahydro-2H-pyran-4-yl)amino)cyclohexyl)amino)-5′,7′-dihydrospiro[pyrrolidine-3,6′-pyrrolo[2,3-d]pyrimidine]-2,5-dione (C154);
7′-cyclopentyl-2′-((3-fluoro-4-isopropylphenyl)amino)-5′,7′-dihydrospiro[pyrrolidine-3,6′-pyrrolo[2,3-d]pyrimidine]-2,5-dione (C155);
7′-cyclopentyl-2′-((4-(6-methyl-2,6-diazaspiro[3.3]heptan-2-yl)phenyl)amino)-5′,7′-dihydrospiro[pyrrolidine-3,6′-pyrrolo[2,3-d]pyrimidin]-2-one (C156);
7′-cyclopentyl-2′-((3-fluoro-4-(6-methyl-2,6-diazaspiro[3.3]heptan-2-yl)phenyl)amino)-5′,7′-dihydrospiro[pyrrolidine-3,6′-pyrrolo[2,3-d]pyrimidin]-2-one (C157);
7′-cyclopentyl-2′-((4-(6-isopropyl-2,6-diazaspiro[3.3]heptan-2-yl)phenyl)amino)-5′,7′-dihydrospiro[pyrrolidine-3,6′-pyrrolo[2,3-d]pyrimidin]-2-one (C158);
7′-cyclopentyl-2′-((3-fluoro-4-(6-isopropyl-2,6-diazaspiro[3.3]heptan-2-yl)phenyl)amino)-5′,7′-dihydrospiro[pyrrolidine-3,6′-pyrrolo[2,3-d]pyrimidin]-2-one (C159);
7′-cyclopentyl-2′-((4-(methylsulfonyl)phenyl)amino)-5′,7′-dihydrospiro[pyrrolidine-3,6′-pyrrolo[2,3-d]pyrimidin]-2-one (C160);
7′-cyclopentyl-2′-((1-isopropylpiperidin-4-yl)amino)-5′,7′-dihydrospiro[pyrrolidine-3,6′-pyrrolo[2,3-d]pyrimidin]-2-one (C161);
7′-cyclopentyl-2′-(((1 r,4r)-4-((tetrahydro-2H-pyran-4-yl)amino)cyclohexyl)amino)-5′,7′-dihydrospiro[pyrrolidine-3,6′-pyrrolo[2,3-d]pyrimidin]-2-one (C162);
7′-cyclopentyl-2′-(((1 S,2S)-2-hydroxy-2-methylcyclopentyl)amino)-5′,7′-dihydrospiro[pyrrolidine-3,6′-pyrrolo[2,3-d]pyrimidin]-2-one (C163);
7′-cyclopentyl-2′-((4-(1-methylpiperidin-4-yl)phenyl)amino)-5′,7′-dihydrospiro[pyrrolidine-3,6′-pyrrolo[2,3-d]pyrimidin]-2-one (C164);
7′-cyclopentyl-2′-((4-(2-morpholinoethoxy)phenyl)amino)-5′,7′-dihydrospiro[pyrrolidine-3,6′-pyrrolo[2,3-d]pyrimidin]-2-one (C165);
7′-cyclopentyl-2′-((3-fluoro-4-morpholinophenyl)amino)-5′,7′-dihydrospiro[pyrrolidine-3,6′-pyrrolo[2,3-d]pyrimidin]-2-one (C166);
7′-cyclopentyl-2′-((6-(2-(dimethylamino)ethoxy)pyridin-3-yl)amino)-5′,7′-dihydrospiro[pyrrolidine-3,6′-pyrrolo[2,3-d]pyrimidin]-2-one (C167);
7′-cyclopentyl-2′-((4-(1-methyl-1H-pyrazol-4-yl)phenyl)amino)-5′,7′-dihydrospiro[pyrrolidine-3,6′-pyrrolo[2,3-d]pyrimidin]-2-one (C168);
7′-cyclopentyl-2′-((4-(1-isopropyl-1H-pyrazol-4-yl)phenyl)amino)-5′,7′-dihydrospiro[pyrrolidine-3,6′-pyrrolo[2,3-d]pyrimidin]-2-one (C169);
7′-cyclopentyl-2′-((6-(2-(pyrrolidin-1-yl)ethoxy)pyridin-3-yl)amino)-5′,7′-dihydrospiro[pyrrolidine-3,6′-pyrrolo[2,3-d]pyrimidin]-2-one (C170);
7′-cyclopentyl-2′-((6-(2-(piperidin-1-yl)ethoxy)pyridin-3-yl)amino)-5′,7′-dihydrospiro[pyrrolidine-3,6′-pyrrolo[2,3-d]pyrimidin]-2-one (C171);
7′-cyclopentyl-2′-((4-((S)-3-(dimethylamino)pyrrolidin-1-yl)phenyl)amino)-5′,7′-dihydrospiro[pyrrolidine-3,6′-pyrrolo[2,3-d]pyrimidin]-2-one (C172);
7′-cyclopentyl-2′-((4-((S)-3-(dimethylamino)pyrrolidin-1-yl)-3-fluorophenyl)amino)-5′,7′-dihydrospiro[pyrrolidine-3,6′-pyrrolo[2,3-d]pyrimidin]-2-one (C173);
7′-cyclopentyl-2′-((6-((S)-3-(dimethylamino)pyrrolidin-1-yl)pyridin-3-yl)amino)-5′,7′-dihydrospiro[pyrrolidine-3,6′-pyrrolo[2,3-d]pyrimidin]-2-one (C174);
7′-cyclopentyl-2′-((4-((R)-3-(dimethylamino)pyrrolidin-1-yl)phenyl)amino)-5′,7′-dihydrospiro[pyrrolidine-3,6′-pyrrolo[2,3-d]pyrimidin]-2-one (C175);
7′-cyclopentyl-2′-((4-((R)-3-(dimethylamino)pyrrolidin-1-yl)-3-fluorophenyl)amino)-5′,7′-dihydrospiro[pyrrolidine-3,6′-pyrrolo[2,3-d]pyrimidin]-2-one (C176);
7′-cyclopentyl-2′-((6-((R)-3-(dimethylamino)pyrrolidin-1-yl)pyridin-3-yl)amino)-5′,7′-dihydrospiro[pyrrolidine-3,6′-pyrrolo[2,3-d]pyrimidin]-2-one (C177);
7′-cyclopentyl-2′-((4-((S)-3-(dimethylamino)pyrrolidine-1-carbonyl)phenyl)amino)-5′,7′-dihydrospiro[pyrrolidine-3,6′-pyrrolo[2,3-d]pyrimidin]-2-one (C178);
7′-cyclopentyl-2′-((4-((S)-3-(dimethylamino)pyrrolidine-1-carbonyl)-3-fluorophenyl)amino)-5′,7′-dihydrospiro[pyrrolidine-3,6′-pyrrolo[2,3-d]pyrimidin]-2-one (C179);
7′-cyclopentyl-2′-((6-((S)-3-(dimethylamino)pyrrolidine-1-carbonyl)pyridin-3-yl)amino)-5′,7′-dihydrospiro[pyrrolidine-3,6′-pyrrolo[2,3-d]pyrimidin]-2-one (C180);
7′-cyclopentyl-2′-((4-((R)-3-(dimethylamino)pyrrolidine-1-carbonyl)phenyl)amino)-5′,7′-dihydrospiro[pyrrolidine-3,6′-pyrrolo[2,3-d]pyrimidin]-2-one (C181);
7′-cyclopentyl-2′-((4-((R)-3-(dimethylamino)pyrrolidine-1-carbonyl)-3-fluorophenyl)amino)-5′,7′-dihydrospiro[pyrrolidine-3,6′-pyrrolo[2,3-d]pyrimidin]-2-one (C182);
7′-cyclopentyl-2′-((6-((R)-3-(dimethylamino)pyrrolidine-1-carbonyl)pyridin-3-yl)amino)-5′,7′-dihydrospiro[pyrrolidine-3,6′-pyrrolo[2,3-d]pyrimidin]-2-one (C183);
7′-cyclopentyl-2′-((4-isopropylphenyl)amino)-5′,7′-dihydrospiro[pyrrolidine-3,6′-pyrrolo[2,3-d]pyrimidin]-2-one (C184);
7′-cyclopentyl-2′-((3-fluoro-4-isopropylphenyl)amino)-5′,7′-dihydrospiro[pyrrolidine-3,6′-pyrrolo[2,3-d]pyrimidin]-2-one (C185);
2′-((4-acetyl-3-fluorophenyl)amino)-7′-cyclopentyl-5′,7′-dihydrospiro[pyrrolidine-3,6′-pyrrolo[2,3-d]pyrimidin]-2-one (C186);
2′-((4-(tert-butyl)-3-fluorophenyl)amino)-7′-cyclopentyl-5′,7′-dihydrospiro[pyrrolidine-3,6′-pyrrolo[2,3-d]pyrimidin]-2-one (C187);
7′-cyclopentyl-2′-((4-hydroxy-4-methylcyclohexyl)amino)-5′,7′-dihydrospiro[pyrrolidine-3,6′-pyrrolo[2,3-d]pyrimidin]-2-one (C188);
7′-cyclopentyl-2′-(((1s,4s)-4-hydroxy-4-methylcyclohexyl)amino)-5′,7′-dihydrospiro[pyrrolidine-3,6′-pyrrolo[2,3-d]pyrimidin]-2-one (C189);
7′-cyclopentyl-2′-(((1 r,4r)-4-hydroxy-4-methylcyclohexyl)amino)-5′,7′-dihydrospiro[pyrrolidine-3,6′-pyrrolo[2,3-d]pyrimidin]-2-one (C190);
7′-cyclopentyl-2′-((5-(2-(dimethylamino)ethoxy)pyridin-2-yl)amino)-5′,7′-dihydrospiro[pyrrolidine-3,6′-pyrrolo[2,3-d]pyrimidin]-2-one (C191);
7′-cyclopentyl-2′-((5-(2-(pyrrolidin-1-yl)ethoxy)pyridin-2-yl)amino)-5′,7′-dihydrospiro[pyrrolidine-3,6′-pyrrolo[2,3-d]pyrimidin]-2-one (C192);
7′-cyclopentyl-2′-((5-(2-(piperidin-1-yl)ethoxy)pyridin-2-yl)amino)-5′,7′-dihydrospiro[pyrrolidine-3,6′-pyrrolo[2,3-d]pyrimidin]-2-one (C193);
(S)-7′-cyclopentyl-2′-(((1S,2S,4R)-1,7,7-trimethylbicyclo[2.2.1]heptan-2-yl)amino)-5′,7′-dihydrospiro[pyrrolidine-3,6′-pyrrolo[2,3-d]pyrimidin]-2-one (C194);
(S)-7′-cyclopentyl-2′-(((1R,2S,4R)-1,7,7-trimethylbicyclo[2.2.1]heptan-2-yl)amino)-5′,7′-dihydrospiro[pyrrolidine-3,6′-pyrrolo[2,3-d]pyrimidin]-2-one (C195);
(S)-7′-cyclopentyl-2′-(((1S,2S,3S,5R)-2,6,6-trimethylbicyclo[3.1.1]heptan-3-yl)amino)-5′,7′-dihydrospiro[pyrrolidine-3,6′-pyrrolo[2,3-d]pyrimidin]-2-one (C196);
7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one (C197);
7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidine]-2,5-dione (C198);
7′-cyclohexyl-2′-((4-(4-methylpiperazin-1-yl)phenyl)amino)-5′,7′-dihydrospiro[pyrrolidine-3,6′-pyrrolo[2,3-d]pyrimidin]-2-one (C199);
7′-cyclohexyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one (C200);
7′-cyclohexyl-2′-((6-(4-methylpiperazin-1-yl)pyridin-3-yl)amino)-5′,7′-dihydrospiro[pyrrolidine-3,6′-pyrrolo[2,3-d]pyrimidin]-2-one (C201);
7′-cyclohexyl-2′-((5-(4-methylpiperazin-1-yl)pyridin-2-yl)amino)-5′,7′-dihydrospiro[pyrrolidine-3,6′-pyrrolo[2,3-d]pyrimidin]-2-one (C202);
7′-cyclopentyl-2′-((3-fluoro-4-(piperazin-1-yl)phenyl)amino)-5′,7′-dihydrospiro[pyrrolidine-3,6′-pyrrolo[2,3-d]pyrimidin]-2-one (C203);
7′-cyclopentyl-2′-((3-fluoro-4-(piperazin-1-yl)phenyl)amino)-5′,7′-dihydrospiro[pyrrolidine-3,6′-pyrrolo[2,3-d]pyrimidin]-2-one (C204);
7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one (C205);
7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one (C206);
7′-cyclopentyl-2′-((4-(3,5-dimethylpiperazin-1-yl)phenyl)amino)-5′,7′-dihydrospiro[pyrrolidine-3,6′-pyrrolo[2,3-d]pyrimidin]-2-one (C207);
7′-cyclopentyl-2′-((4-(3,5-dimethylpiperazin-1-yl)phenyl)amino)-5′,7′-dihydrospiro[pyrrolidine-3,6′-pyrrolo[2,3-d]pyrimidine]-2,5-dione (C208);
7′-cyclopentyl-2′-((4-isopropylphenyl)amino)-5′,7′-dihydrospiro[pyrrolidine-3,6′-pyrrolo[2,3-d]pyrimidin]-2-one (C209);
7′-cyclopentyl-2′-((5-(2-(dimethylamino)ethoxy)pyridin-2-yl)amino)-5′,7′-dihydrospiro[pyrrolidine-3,6′-pyrrolo[2,3-d]pyrimidin]-2-one (C210);
tert-butyl(S)-7′-cyclopentyl-2′-(methylthio)-2-oxo-5′,7′-dihydrospiro[pyrrolidine-3,6′-pyrrolo[2,3-d]pyrimidine]-1-carboxylate (C211);
tert-butyl(S)-7′-cyclohexyl-2′-(methylthio)-2-oxo-5′,7′-dihydrospiro[pyrrolidine-3,6′-pyrrolo[2,3-d]pyrimidine]-1-carboxylate (C212); or
pharmaceutically acceptable salts thereof.

### DESCRIPTION OF THE DRAWINGS

FIG. 1 shows CDK4 inhibition data for Compounds C1-C107. The graph excludes all results with IC₅₀ values greater than 25 μM. The plotted data is shown in Table 2.

FIG. 2 shows CDK6 inhibition data for Compounds C1-C107. The graph excludes all results with IC₅₀ values greater than 25 μM. The plotted data is shown in Table 2.

FIG. 3 shows thermal ellipsoid plot¹ of VP-8-69E1. (¹ Dolomanov, O. V.; Bourhis, L. J; Gildea, R. J.; Howard, J. A. K; Puschman, H., OLEX2: A complete structure solution, refinement and analysis program (2009). J. Appl. Cryst., 42, 339-341).

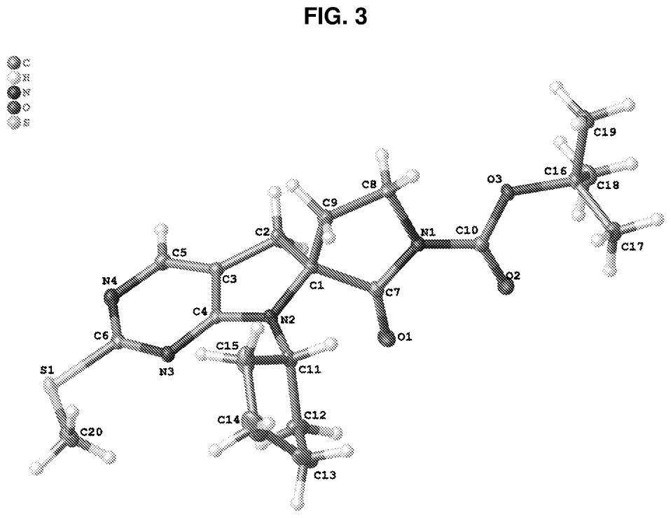

FIG. 4 shows thermal ellipsoid plot¹ of VP-7-235E1. (¹ Dolomanov, O. V.; Bourhis, L. J; Gildea, R. J.; Howard, J. A. K; Puschman, H., OLEX2: A complete structure solution, refinement and analysis program (2009). J. Appl. Cryst., 42, 339-341)

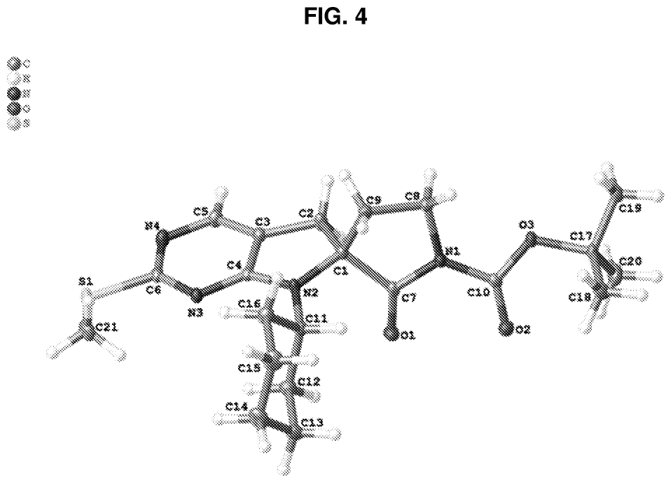

### DETAILED DESCRIPTION

Described herein are compounds that are useful for treating uncontrolled cell proliferative diseases, including, but not limited to, proliferative diseases such as cancer, restenosis, or rheumatoid arthritis. These compounds are useful for treating inflammation and inflammatory diseases. In addition, these compounds have utility as antiinfective agents. Moreover, these compounds have utility as chemoprotective agents through their ability to inhibit the cell cycle progression of normal untransformed cells. Many of the compounds described herein display unexpected improvements in selectivity for the serine/threonine kinases CDK4, CDK6, CDK9, and other CDKs. The synthesis of these compounds is described herein. These compounds can be administered to patients by a variety of methods including orally or intravenously.

Throughout the specification and claims, a given chemical formula or chemical name shall encompass all optical stereoisomers, as well as racemic mixtures where such isomers or mixtures exist, unless the specific isomer or diastereomer is noted. In addition, the disclosed compounds encompass all pharmaceutically acceptable salts, esters, amides, isotopes, prodrugs, solvates, or crystalline forms thereof.

The term “alkyl” as used herein means a straight or branched hydrocarbon radical having from 1 to 10 carbon atoms and includes, for example, methyl, ethyl, n-propyl, isopropyl, n-butyl, sec-butyl, isobutyl, tert-butyl, n-pentyl, iso-pentyl, n-hexyl, and the like.

The term “alkenyl” as used means straight and branched hydrocarbon radicals having from 2 to 8 carbon atoms and at least one double bond and includes, but is not limited to, ethenyl, 3-buten-1-yl, 2-ethenylbutyl, 3-hexen-1-yl, and the like. The term “alkenyl” includes cycloalkenyl, and heteroalkenyl in which 1 to 3 heteroatoms selected from O, S, N, or substituted nitrogen may replace carbon atoms.

The term “alkynyl” as used means straight and branched hydrocarbon radicals having from 2 to 8 carbon atoms and at least one triple bond and includes, but is not limited to, ethynyl, 3-butyn-1-yl, propynyl, 2-butyn-1-yl, 3-pentyn-1-yl, and the like.

The term “cycloalkyl” as used means a monocyclic or polycyclic hydrocarbyl group having from 3 to 8 carbon atoms, for instance, cyclopropyl, cycloheptyl, cyclooctyl, cyclodecyl, cyclobutyl, adamantyl, norpinanyl, decalinyl, norbornyl, cyclohexyl, and cyclopentyl. Such groups can be substituted with groups such as hydroxy, keto, amino, alkyl, and dialkylamino, and the like. Also included are rings in which 1 to 3 heteroatoms replace carbons. Such groups are termed “heterocyclyl,” which means a cycloalkyl group also bearing at least one heteroatom selected from O, S, N, or substituted nitrogen. Examples of such groups include, but are not limited to, oxiranyl, pyrrolidinyl, piperidyl, tetrahydropyran, and morpholine.

The term “alkoxy” as used herein means a straight or branched chain alkyl groups having 1-10 carbon atoms and linked through oxygen. Examples of such groups include, but are not limited to, methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, and 3-methylpentoxy. In addition, alkoxy refers to polyethers such as —O—(CH₂)₂—O—CH₃, and the like.

The alkyl, alkenyl, alkoxy, and alkynyl groups described herein are optionally substituted, preferably by 1 to 3 groups selected from NR4R5, phenyl, substituted phenyl, thio C₁-C₆ alkyl, C₁-C₆ alkoxy, hydroxy, carboxy, C₁-C₆ alkoxycarbonyl, halo, nitrile, cycloalkyl, and a 5- or 6-membered carbocyclic ring or heterocyclic ring having 1 or 2 heteroatoms selected from nitrogen, substituted nitrogen, oxygen, and sulfur. “Substituted nitrogen” means nitrogen bearing C1-C₆ alkyl or (CH₂)ₚPh where p is 1, 2, or 3. Perhalo and polyhalo substitution is also included.

Examples of substituted alkyl groups include, but are not limited to, 2-aminoethyl, 2-hydroxyethyl, pentachloroethyl, trifluoromethyl, 2-diethylaminoethyl, 2-dimethylaminopropyl, ethoxycarbonylmethyl, 3-phenylbutyl, methanylsulfanylmethyl, methoxymethyl, 3-hydroxypentyl, 2-carboxybutyl, 4-chlorobutyl, 3-cyclopropylpropyl, pentafluoroethyl, 3-morpholinopropyl, piperazinylmethyl, and 2-(4-methylpiperazinyl)ethyl.

Examples of substituted alkynyl groups include, but are not limited to, 2-methoxyethynyl, 2-ethylsulfanylethynyl, 4-(1-piperazinyl)-3-(butynyl), 3-phenyl-5-hexynyl, 3-diethylamino-3-butynyl, 4-chloro-3-butynyl, 4-cyclobutyl-4-hexenyl, and the like.

Typical substituted alkoxy groups include aminomethoxy, trifluoromethoxy, 2-diethylaminoethoxy, 2-ethoxycarbonylethoxy, 3-hydroxypropoxy, 6-carboxhexyloxy, and the like.

Further, examples of substituted alkyl, alkenyl, and alkynyl groups include, but are not limited to, dimethylaminomethyl, carboxymethyl, 4-dimethylamino-3-buten-1-yl, 5-ethylmethylamino-3-pentyn-1-yl, 4-morpholinobutyl, 4-tetrahydropyrinidylbutyl, 3-imidazolidin-1-ylpropyl, 4-tetrahydrothiazol-3-yl-butyl, phenylmethyl, 3-chlorophenylmethyl, and the like.

The term “anion” as used herein means a negatively charged counterion such as chloride, bromide, trifluoroacetate, and triethylammonium.

The term “acyl” as used herein means an alkyl or aryl (Ar) group having from 1-10 carbon atoms bonded through a carbonyl group, i.e., R—C(O)—. For example, acyl includes, but is not limited to, a C₁-C₆ alkanoyl, including substituted alkanoyl, wherein the alkyl portion can be substituted by an amine, amide, carboxylic, or heterocyclic group. Typical acyl groups include acetyl, benzoyl, and the like.

The term “aryl” as used herein refers to an aromatic monocyclic hydrocarbon ring system or a polycyclic ring system where at least one of the rings in the ring system is an aromatic hydrocarbon ring and any other aromatic rings in the ring system include only hydrocarbons. In some embodiments, a monocyclic aryl group can have from 6 to 14 carbon atoms and a polycyclic aryl group can have from 8 to 14 carbon atoms. The aryl group can be covalently attached to the defined chemical structure at any carbon atom(s) that result in a stable structure. In some embodiments, an aryl group can have only aromatic carbocyclic rings, e.g., phenyl, 1-naphthyl, 2-naphthyl, anthracenyl, phenanthrenyl groups, and the like. In other embodiments, an aryl group can be a polycyclic ring system in which at least one aromatic carbocyclic ring is fused (i.e., having a bond in common with) to one or more cycloalkyl or cycloheteroalkyl rings. Examples of such aryl groups include, among others, benzo derivatives of cyclopentane (i.e., an indanyl group, which is a 5,6-bicyclic cycloalkyl/aromatic ring system), cyclohexane (i.e., a tetrahydronaphthyl group, which is a 6,6-bicyclic cycloalkyl/aromatic ring system), imidazoline (i.e., a benzimidazolinyl group, which is a 5,6-bicyclic cycloheteroalkyl/aromatic ring system), and pyran (i.e., a chromenyl group, which is a 6,6-bicyclic cycloheteroalkyl/aromatic ring system). Other examples of aryl groups include benzodioxanyl, benzodioxolyl, chromanyl, indolinyl groups, and the like.

The terms “halogen” or “halo” as used herein means fluorine, bromine, chlorine, and iodine.

The term “haloalkyl” refers to an alkyl group having one or more halogen substituents. In some embodiments, a haloalkyl group can have 1 to 10 carbon atoms (e.g., from 1 to 8 carbon atoms). Examples of haloalkyl groups include CF₃, C₂F5, CHF₂, CH₂F, CCl₃, CHCl₂, CH₂Cl, C₂Cl₅, and the like. Perhaloalkyl groups, i.e., alkyl groups wherein all of the hydrogen atoms are replaced with halogen atoms (e.g., CF₃ and C₂F₅), are included within the definition of “haloalkyl.” For example, a C₁₋₁₀ haloalkyl group can have the formula —CᵢH₂ᵢ+1−jXⱼ, wherein X is F, Cl, Br, or I, i is an integer in the range of 1 to 10, and j is an integer in the range of 0 to 21, provided that j is less than or equal to 2i+1.

The term “heteroaryl” as used herein refers to an aromatic monocyclic ring system containing at least one ring heteroatom selected from O, N, and S or a polycyclic ring system where at least one of the rings in the ring system is aromatic and contains at least one ring heteroatom. A heteroaryl group, as a whole, can have from 5 to 14 ring atoms and contain 1-5 ring heteroatoms. In some embodiments, heteroaryl groups can include monocyclic heteroaryl rings fused to one or more aromatic carbocyclic rings, non-aromatic carbocyclic rings, or non-aromatic cycloheteroalkyl rings. The heteroaryl group can be covalently attached to the defined chemical structure at any heteroatom or carbon atom that results in a stable structure. Generally, heteroaryl rings do not contain O—O, S—S, or S—O bonds. However, one or more N or S atoms in a heteroaryl group can be oxidized (e.g., pyridine N-oxide, thiophene S-oxide, thiophene S,S-dioxide). Examples of such heteroaryl rings include pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzothiazolyl, benzisothiazolyl, benzisoxazolyl, benzoxadiazolyl, benzoxazolyl, cinnolinyl, 1H-indazolyl, 2H-indazolyl, indolizinyl, isobenzofuyl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyrdazinyl, thienothiazolyl, thienooxazolyl, thienoimidazolyl groups, and the like. Further examples of heteroaryl groups include 4,5,6,7-tetrahydroindolyl, tetrahydroquinolinyl, benzothienopyridinyl, benzofuropyridinyl groups, and the like.

The term “lower alkenyl” as used herein refers to alkenyl groups which contains 2 to 6 carbon atoms. An alkenyl group is a hydrocarbyl group containing at least one carbon-carbon double bond. As defined herein, it may be unsubstituted or substituted with the substituents described herein. The carbon-carbon double bonds may be between any two carbon atoms of the alkenyl group. It is preferred that it contains 1 or 2 carbon-carbon double bonds and more preferably one carbon-carbon double bond. The alkenyl group may be straight chained or branched. Examples include but are not limited to ethenyl, 1-propenyl, 2-propenyl, 1-butenyl, 2-butenyl, 2-methyl-1-propenyl, 1,3-butadienyl, and the like.

The term “lower alkynyl” as used herein, refers to an alkynyl group containing 2-6 carbon atoms. An alkynyl group is a hydrocarbyl group containing at least one carbon-carbon triple bond. The carbon-carbon triple bond may be between any two carbon atom of the alkynyl group. In an embodiment, the alkynyl group contains 1 or 2 carbon-carbon triple bonds and more preferably one carbon-carbon triple bond. The alkynyl group may be straight chained or branched. Examples include but are not limited to ethynyl, 1-propynyl, 2-propynyl, 1-butynyl, 2-butynyl and the like.

The term “carbalkoxy” as used herein refers to an alkoxycarbonyl group, where the attachment to the main chain is through the carbonyl group, e.g., —C(O)—. Examples include but are not limited to methoxy carbonyl, ethoxy carbonyl, and the like.

The term “oxo” as used herein refers to a double-bonded oxygen (i.e., ═O). It is also to be understood that the terminology C(O) refers to a —C═O group, whether it be ketone, aldehyde or acid or acid derivative. Similarly, S(O) refers to a —S═O group.

The term “cycloalkyl” as used herein refers to a non-aromatic carbocyclic group including cyclized alkyl, alkenyl, and alkynyl groups. A cycloalkyl group can be monocyclic (e.g., cyclohexyl) or polycyclic (e.g., containing fused, bridged, and/or spiro ring systems), wherein the carbon atoms are located inside or outside of the ring system. A cycloalkyl group, as a whole, can have from 3 to 14 ring atoms (e.g., from 3 to 8 carbon atoms for a monocyclic cycloalkyl group and from 7 to 14 carbon atoms for a polycyclic cycloalkyl group). Any suitable ring position of the cycloalkyl group can be covalently linked to the defined chemical structure. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcaryl, adamantyl, and spiro[4.5]decanyl groups, as well as their homologs, isomers, and the like.

The term “heteroatom” as used herein refers to an atom of any element other than carbon or hydrogen and includes, for example, nitrogen, oxygen, sulfur, phosphorus, and selenium.

The term “cycloheteroalkyl” as used herein refers to a non-aromatic cycloalkyl group that contains at least one (e.g., one, two, three, four, or five) ring heteroatom selected from O, N, and S, and optionally contains one or more (e.g., one, two, or three) double or triple bonds. A cycloheteroalkyl group, as a whole, can have from 3 to 14 ring atoms and contains from 1 to 5 ring heteroatoms (e.g., from 3-6 ring atoms for a monocyclic cycloheteroalkyl group and from 7 to 14 ring atoms for a polycyclic cycloheteroalkyl group). The cycloheteroalkyl group can be covalently attached to the defined chemical structure at any heteroatom(s) or carbon atom(s) that results in a stable structure. One or more N or S atoms in a cycloheteroalkyl ring may be oxidized (e.g., morpholine N-oxide, thiomorpholine S-oxide, thiomorpholine S,S-dioxide). Cycloheteroalkyl groups can also contain one or more oxo groups, such as phthalimidyl, piperidonyl, oxazolidinonyl, 2,4(1H,3H)-dioxo-pyrimidinyl, pyridin-2(1H)-onyl, and the like. Examples of cycloheteroalkyl groups include, among others, morpholinyl, thiomorpholinyl, pyranyl, imidazolidinyl, imidazolinyl, oxazolidinyl, pyrazolidinyl, pyrazolinyl, pyrrolidinyl, pyrrolinyl, tetrahydrofuranyl, tetrahydrothienyl, piperidinyl, piperazinyl, azetidine, and the like.

The compounds described herein may contain chiral centers and therefore may exist in different enantiomeric and diastereomeric forms. One embodiment described herein encompasses all optical isomers or stereoisomers of the compounds described herein both as racemic mixtures and as individual enantiomers or diastereoisomers, or mixtures thereof, and to all pharmaceutical compositions o methods of treatment described herein that contain or employ them, respectively. Individual isomers can be obtained by known methods, such as optical resolution, optically selective reaction, or chiral chromatographic separation in the preparation of the final product or its intermediate.

The compounds described herein can exist in unsolvated forms as well as solvated forms, including hydrated forms. In general, the solvated forms, including hydrated forms, are equivalent to unsolvated forms and are intended to be encompassed within the scope described herein.

Compounds described herein also includes isotopically labelled compounds, which are identical to those described herein, but for the fact that one or more atoms are replaced by an atom having an atomic mass or mass number different from the atomic mass or mass number usually found in nature. Examples of isotopes that can be incorporated into compounds described herein include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds described herein, prodrugs thereof, and pharmaceutically acceptable salts of said compounds or of said prodrugs which contain the aforementioned isotopes and/or other isotopes of other atoms are within the scope described herein. Certain isotopically labelled compounds described herein, for example those into which radioactive isotopes such as ³H and ¹⁴C are incorporated, are useful in drug and/or substrate tissue distribution assays. Tritiated, i.e., ³H, and carbon-14 i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds described herein and prodrugs thereof can generally be prepared by carrying out the procedures disclosed in the Schemes and/or in the Examples and Preparations below, by substituting a readily available isotopically labelled reagent for a non-isotopically labelled reagent.

The term “cancer” as used herein includes, but is not limited to, the following cancers: cancers of the breast, ovary, cervix, prostate, testis, esophagus, stomach, skin, lung, bone, colon, pancreas, thyroid, biliary passages, buccal cavity and pharynx (oral), lip, tongue, mouth, pharynx, small intestine, colon-rectum, large intestine, rectum, brain and central nervous system, glioblastoma, neuroblastoma, keratoacanthoma, epidernoid carcinoma, large cell carcinoma, adenocarcinoma, adenocarcinoma, adenoma, adenocarcinoma, follicular carcinoma, undifferentiated carcinoma, papillary carcinoma, seminoma, melanoma, sarcoma, bladder carcinoma, liver carcinoma, kidney carcinoma, myeloid disorders, lymphoid disorders, Hodgkin's, hairy cells, and leukemia.

The term “treating,” as used herein refers to reversing, alleviating, inhibiting the progress of, or preventing the disorder or condition to which such term applies, or ameliorating one or more symptoms of such condition or disorder. The term “treatment,” as used herein, refers to the act of treating, as “treating” is defined immediately above.

The phrase “pharmaceutically acceptable” refers to molecular entities and compositions that are physiologically tolerable and do not typically produce a toxic, allergic, or similar untoward reaction, such as gastric upset, dizziness and the like, when administered to a human. Preferably, as used herein, the term “pharmaceutically acceptable” means approved by a regulatory agency of the Federal or a state government or listed in the U.S. or European Pharmacopeia or other generally recognized pharmacopeia for use in animals, and more particularly in humans.

The compounds described herein are capable of further forming pharmaceutically acceptable formulations comprising salts, including acid addition or base salts, solvents, or N-oxides of any compound described herein.

The term “salts” refers to the relatively non-toxic, inorganic, or organic acid addition salts of compounds described herein. These salts can be prepared in situ during the final isolation and purification of the compounds or by separately reacting the purified compound in its free base form with a suitable organic or inorganic acid and isolating the salt thus formed. In so far as the compounds described herein are basic compounds, they are all capable of forming a wide variety of different salts with various inorganic and organic acids. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent and thereafter convert the free base to a pharmaceutically acceptable acid addition salt. The acid addition salts of the basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of this disclosure.

Pharmaceutically acceptable base addition salts are formed with metals or amines, such as alkali and alkaline earth metal hydroxides, or of organic amines. Examples of metals used as cations are sodium, potassium, magnesium, calcium, and the like. Examples of suitable amines are N,N′-dibenzylethylenediamine, chloroprocaine, choline, diethanolamine, ethylenediamine, N-methylglucamine, and procaine.

The base addition salts of acidic compounds are prepared by contacting the free acid form with a sufficient amount of the desired base to produce the salt in the conventional manner. The free acid form may be regenerated by contacting the salt form with an acid and isolating the free acid in a conventional manner. The free acid forms differ from their respective salt forms somewhat in certain physical properties such as solubility in polar solvents, but otherwise the salts are equivalent to their respective free acid for purposes described herein.

Salts may be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide such as hydrochloric, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts may also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts may include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See Berge et al., J. Pharm. Sci. 66: 1-19 (1977) which is incorporated herein by reference.

The term “pharmaceutically acceptable salts, esters, amides, and prodrugs” as used herein refers to those carboxylate salts, amino acid addition salts, esters, amides, and prodrugs of the compounds described herein which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds described herein.

Examples of pharmaceutically acceptable, non-toxic esters of the compounds described herein include C₁-C₆ alkyl esters wherein the alkyl group is a straight or branched chain. Acceptable esters also include C₅-C₇ cycloalkyl esters as well as arylalkyl esters such as, but not limited to benzyl. C1-C₄ alkyl esters are preferred. Esters of the compounds described herein may be prepared according to conventional methods “March's Advanced Organic Chemistry, 5^{th} Edition,” Smith and March, John Wiley & Sons (2001).

Examples of pharmaceutically acceptable, non-toxic amides of the compounds described herein include amides derived from ammonia, primary C₁-C₆ alkyl amines and secondary C₁-C₆ dialkyl amines wherein the alkyl groups are straight or branched chain. In the case of secondary amines the amine may also be in the form of a 5- or 6-membered heterocycle containing one nitrogen atom. Amides derived from ammonia, C₁-C₃ alkyl primary amines and C₁-C₂ dialkyl secondary amines are preferred. Amides of the compounds described herein may be prepared according to conventional methods such as “March's Advanced Organic Chemistry, 5^{th} Edition,” Smith and March, John Wiley & Sons (2001).

The phrase “room temperature,” “RT,” or “ambient temperature” indicate a temperature of about 25° C.±10%.

The term “prodrug” as used herein refers to compounds that are rapidly transformed in vivo to yield the parent compound of the above formulae, for example, by hydrolysis in blood. A thorough discussion is provided in Higuchi, “Pro-drugs as Novel Delivery Systems,” ACS Symposium Series 14 (1975), and in Roche, Edward, ed., Bioreversible Carriers in Drug Design, American Pharmaceutical Association, Pergamon Press (1987), both of which are hereby incorporated by reference.

The term “excipient” as used herein refers to a diluent, adjuvant, or vehicle with which the compound is administered. Such pharmaceutical excipients can be sterile liquids, such as water and oils, including those of petroleum, animal, vegetable or synthetic origin, such as peanut oil, soybean oil, mineral oil, sesame oil and the like. Water for injection, aqueous saline solutions, aqueous dextrose, or lactated Ringer's solution are preferably employed as excipients, particularly for injectable solutions. Suitable pharmaceutical excipients are described in “Remington's Pharmaceutical Sciences” by E. W. Martin.

The phrase “therapeutically effective amount” as used herein means an amount sufficient to reduce by at least about 10 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition/symptom in the host.

The term “analog” as used herein refers to a small organic compound, a nucleotide, a protein, or a polypeptide that possesses similar or identical activity or function(s) as the compound, nucleotide, protein or polypeptide or compound having the desired activity and therapeutic effect (e.g., inhibition of tumor growth), but need not necessarily comprise a sequence or structure that is similar or identical to the sequence or structure of the preferred embodiment.

The term “derivative” as used herein refers to either a compound, a protein or polypeptide that comprises an amino acid sequence of a parent protein or polypeptide that has been altered by the introduction of amino acid residue substitutions, deletions or additions, or a nucleic acid or nucleotide that has been modified by either introduction of nucleotide substitutions or deletions, additions or mutations. The derivative nucleic acid, nucleotide, protein, or polypeptide possesses a similar or identical function as the parent polypeptide.

The phrases “substantial portion” or “significant portion” as used herein mean at least 80%. In alternative embodiments, the portion may be at least 85%, 90%, 95%, 99%, or greater.

The phrase “selective CDK inhibitor” used in the context of the compounds described herein indicates an ability to inhibit CDK4 activity, CDK6 activity, CDK9 activity or other CDK activities at an IC₅₀ molar concentration at least about 2000 times less than the IC₅₀ molar concentration necessary to inhibit to the same degree as CDK2 activity in a standard phosphorylation assay.

The phrase “induces G1-arrest” mean that a compound described herein induces a quiescent state in a substantial portion of a cell population at the G1 phase of the cell cycle.

The phrase “hematological deficiency” mean reduced hematological cell lineage counts or the insufficient production of blood cells (i.e., myelodysplasia) and/or lymphocytes (i.e., lymphopenia, the reduction in the number of circulating lymphocytes, such as B- and T-cells).

Hematological deficiency can be observed, for example, as myelosuppression in form of anemia, reduction in platelet count (i.e., thrombocytopenia), reduction in white blood cell count (i.e., leukopenia), or the reduction in granulocytes (e.g., neutropenia).

The phrase “synchronous reentry into the cell cycle” mean that CDK4/6-replication dependent healthy cells, for example HSPCs, in G1-arrest due to the effect of a compound described herein reenter the cell-cycle within relatively the same collective timeframe or at relatively the same rate upon dissipation of the compound's effect. Comparatively, by “asynchronous reentry into the cell cycle” is meant that the healthy cells, for example HSPCs, in G1 arrest due to the effect of a CDK4/6 inhibitor compound within relatively different collective timeframes or at relatively different rates upon dissipation of the compound's effect such as pablociclib or ribociclib.

The phrases “off-cycle” or “drug holiday” mean a time period during which the subject is not administered or exposed to a chemotherapeutic. For example, in a treatment regime wherein the subject is administered the chemotherapeutic in a repeated 21 day cycle, and is not administered the chemotherapeutic at the start of the next 21-day cycle due to hematologic deficiencies, the delayed period of non-administration is considered the “off-cycle” or “drug holiday.” Off-target and drug holiday may also refer to an interruption in a treatment regime wherein the subject is not administered the chemotherapeutic for a time due to a deleterious side effect, for example, myelosuppression or other hematological deficiencies.

The phrase “CDK4/6-replication independent cancer” refers to a cancer that does not significantly require the activity of CDK4/6 for replication. Cancers of such type are often, but not always, characterized by (e.g., has cells that exhibit) an increased level of CDK2 activity or by reduced expression of retinoblastoma tumor suppressor protein or retinoblastoma family member protein(s), such as, but not limited to p107 and p130. The increased level of CDK2 activity or reduced or deficient expression of retinoblastoma tumor suppressor protein or retinoblastoma family member protein(s) can be increased or reduced, for example, compared to normal cells. In some embodiments, the increased level of CDK2 activity can be associated with (e.g., can result from or be observed along with) MYC proto-oncogene amplification or overexpression. In some embodiments, the increased level of CDK2 activity can be associated with overexpression of Cyclin E1, Cyclin E2, or Cyclin A.

The phrase “long-term hematological toxicity” is meant hematological toxicity affecting a subject for a period lasting more than one or more weeks, months, or years following administration of a chemotherapeutic agent. Long-term hematological toxicity can result in bone marrow disorders that can cause the ineffective production of blood cells (i.e., myelodysplasia) and/or lymphocytes (i.e., lymphopenia, the reduction in the number of circulating lymphocytes, such as B- and T-cells). Hematological toxicity can be observed, for example, as anemia, reduction in platelet count (i.e., thrombocytopenia) or reduction in white blood cell count (i.e., neutropenia). In some cases, myelodysplasia can result in the development of leukemia. Long-term toxicity related to chemotherapeutic agents can also damage other self-renewing cells in a subject, in addition to hematological cells. Thus, long-term toxicity can also lead to graying and frailty.

### Biological Activity

The compounds described herein are inhibitors of cyclin dependent kinases. For example, compounds described herein are inhibitors of cyclin dependent kinases, and in particular cyclin dependent kinases comprising CDK1, CDK2, CDK3, CDK4, CDK5, CDK6, CDK7, CDK8, CDK9, CDK9, CDK10, CDK11, CDK12, CDK13, or other CDKs. In one aspect, the compounds described herein are inhibitors of CDK4, CDK6, and/or CDK9.

Compounds described herein also have activity against glycogen synthase kinase-3 (GSK-3). As a consequence of their activity in modulating or inhibiting CDK and glycogen synthase kinase, they are expected to be useful in providing a means of arresting, or recovering control of, the cell cycle in abnormally dividing cells. It is therefore anticipated that the compounds will prove useful in treating or preventing proliferative disorders such as cancers. It is also envisaged that the compounds described herein will be useful in treating conditions such as viral infections, type II or non-insulin dependent diabetes mellitus, autoimmune diseases, head trauma, stroke, epilepsy, neurodegenerative diseases such as Alzheimer's, motor neuron disease, progressive supranuclear palsy, corticobasal degeneration and Pick's disease for example autoimmune diseases and neurodegenerative diseases.

One sub-group of disease states and conditions where it is envisaged that the compounds described herein will be useful consists of viral infections, autoimmune diseases and neurodegenerative diseases.

CDKs play a role in the regulation of the cell cycle, apoptosis, transcription, differentiation, and CNS function. Therefore, CDK inhibitors could be useful in the treatment of diseases in which there is a disorder of proliferation, apoptosis, or differentiation such as cancer. In particular, RB-ve tumours may be particularly sensitive to CDK inhibitors. These include tumours harbouring mutations in ras, Raf, Growth Factor Receptors or over-expression of Growth Factor Receptors. Furthermore, tumours with hypermethylated promoter regions of CDK inhibitors as well as tumours over-expressing cyclin partners of the cyclin dependent kinases may also display sensitivity. RB-ve tumours may also be sensitive to CDK inhibitors.

Examples of cancers which may be inhibited include, but are not limited to, a carcinoma, for example a carcinoma of the bladder, breast, colon (e.g., colorectal carcinomas such as colon adenocarcinoma and colon adenoma), kidney, epidermis, liver, lung, for example adenocarcinoma, small cell lung cancer and non-small cell lung carcinomas, oesophagus, gall bladder, ovary, pancreas e.g., exocrine pancreatic carcinoma, stomach, cervix, thyroid, nose, head and neck, prostate, or skin, for example squamous cell carcinoma; a hematopoietic tumour of lymphoid lineage, for example leukemia, acute lymphocytic leukemia, chronic lymphocytic leukaemia, B-cell lymphoma (such as diffuse large B cell lymphoma), T-cell lymphoma, multiple myeloma, Hodgkin's lymphoma, non-Hodgkin's lymphoma, hairy cell lymphoma, or Burkett's lymphoma; a hematopoietic tumour of myeloid lineage, for example acute and chronic myelogenous leukemias, myelodysplastic syndrome, or promyelocytic leukemia; thyroid follicular cancer; a tumour of mesenchymal origin, for example fibrosarcoma or habdomyosarcoma; a tumour of the central or peripheral nervous system, for example astrocytoma, neuroblastoma, glioma or schwannoma; melanoma; seminoma; teratocarcinoma; osteosarcoma; xeroderma pigmentosum; keratoctanthoma; thyroid follicular cancer; or Kaposi's sarcoma.

The cancers may be cancers which are sensitive to inhibition of any one or more cyclin dependent kinases comprising CDK1, CDK2, CDK3, CDK4, CDK5, CDK6, CDK7, CDK8, CDK9, CDK9, CDK10, CDK11, CDK12, CDK13, or other CDKs for example, one or more CDK kinases selected from CDK4, CDK6, and/or CDK9. Whether or not a particular cancer is one which is sensitive to inhibition by a cyclin dependent kinase inhibitor may be determined by means of a cell growth assay.

CDKs are also known to play a role in apoptosis, proliferation, differentiation and transcription and therefore CDK inhibitors could also be useful in the treatment of the following diseases other than cancer; viral infections, for example herpes virus, pox virus, Epstein-Barr virus, Sindbis virus, adenovirus, HIV, HPV, HCV and HCMV; prevention of AIDS development in HIV-infected individuals; chronic inflammatory diseases, for example systemic lupus erythematosus, autoimmune mediated glomerulonephritis, rheumatoid arthritis, psoriasis, inflammatory bowel disease, and autoimmune diabetes mellitus; cardiovascular diseases for example cardiac hypertrophy, restenosis, atherosclerosis; neurodegenerative disorders, for example Alzheimer's disease, AIDS-related dementia, Parkinson's disease, amyotrophic lateral sclerosis, retinitis pigmentosa, spinal muscular atrophy and cerebellar degeneration; glomerulonephritis; myelodysplastic syndromes, ischemic injury associated myocardial infarctions, stroke and reperfusion injury, arrhythmia, atherosclerosis, toxin-induced or alcohol related liver diseases, haematological diseases, for example, chronic anemia and aplastic anemia; degenerative diseases of the musculoskeletal system, for example, osteoporosis and arthritis, aspirin-sensitive rhinosinusitis, cystic fibrosis, multiple sclerosis, kidney diseases, ophthalmic diseases including age related macular degeneration, uveitis, and cancer pain.

It has also been discovered that some cyclin-dependent kinase inhibitors can be used in combination with other anticancer agents. For example, the cyclin-dependent kinase inhibitor flavopiridol has been used with other anticancer agents in combination therapy. Thus, in the pharmaceutical compositions, uses or methods described herein for treating a disease or condition comprising abnormal cell growth, the disease or condition comprising abnormal cell growth in one embodiment is a cancer.

One group of cancers includes human breast cancers (e.g., primary breast tumours, node-negative breast cancer, invasive duct adenocarcinomas of the breast, non-endometrioid breast cancers); and mantle cell lymphomas. In addition, other cancers are colorectal and endometrial cancers. Another sub-set of cancers includes hematopoietic tumours of lymphoid lineage, for example leukemia, chronic lymphocytic leukaemia, mantle cell lymphoma, and B-cell lymphoma (such as diffuse large B cell lymphoma). One particular cancer is chronic lymphocytic leukaemia. Another particular cancer is mantle cell lymphoma. Another particular cancer is diffuse large B cell lymphoma. Another sub-set of cancers includes breast cancer, ovarian cancer, colon cancer, prostate cancer, oesophageal cancer, squamous cancer, and non-small cell lung carcinomas. Another sub-set of cancers includes breast cancer, pancreatic cancer, colorectal cancer, lung cancer, and melanoma.

A further sub-set of cancers, namely cancers wherein compounds having CDK4 inhibitory activity may be of particular therapeutic benefit, comprises retinoblastomas, small cell lung carcinomas, non-small lung carcinomas, sarcomas, gliomas, pancreatic cancers, head, neck and breast cancers and mantle cell lymphomas.

Another sub-set of cancers wherein compounds having CDK4 inhibitory activity may be of particular therapeutic benefit comprises small cell lung cancer, non-small cell lung cancer, pancreatic cancer, breast cancer, glioblastoma multiforme, T cell acute lymphoblastic leukaemia and mantle cell lymphoma. A further subset of cancers which the compounds described herein may be useful in the treatment of includes sarcomas, leukemias, glioma, familial melanoma and melanoma.

One embodiment described herein is a method of treating a disorder or condition selected from the group consisting of cell proliferative disorders, such as cancer, vascular smooth muscle proliferation associated with atherosclerosis, postsurgical vascular stenosis, restenosis, and endometriosis; infections, including viral infections such as DNA viruses like herpes and RNA viruses like HIV, and fungal infections; autoimmune diseases such as psoriasis, inflammation like rheumatoid arthritis, lupus, type 1 diabetes, diabetic nephropathy, multiple sclerosis, and glomerulonephritis, organ transplant rejection, including host versus graft disease, in a mammal, including human, comprising administering to said mammal an amount of a compound described herein, or a pharmaceutically acceptable salt thereof, that is effective in treating such disorder or condition.

Another embodiment described herein provides compounds that are useful for treating abnormal cell proliferation such a cancer. One aspect is a method of treating the abnormal cell proliferation disorders such as a cancer selected from the group consisting of cancers of the breast, ovary, cervix, prostate, testis, esophagus, stomach, skin, lung, bone, colon, pancreas, thyroid, biliary passages, buccal cavity and pharynx (oral), lip, tongue, mouth, pharynx, small intestine, colon-rectum, large intestine, rectum, brain and central nervous system, glioblastoma, neuroblastoma, keratoacanthoma, epidermoid carcinoma, large cell carcinoma, adenocarcinoma, adenocarcinoma, adenoma, adenocarcinoma, follicular carcinoma, undifferentiated carcinoma, papillary carcinoma, seminoma, melanoma, sarcoma, bladder carcinoma, liver carcinoma, kidney carcinoma, myeloid disorders, lymphoid disorders, Hodgkin's, hairy cells, and leukemia, comprising administering a therapeutically effective amount of a compound described herein, or a pharmaceutically acceptable salt thereof, to a subject in need of such treatment.

A further embodiment described herein is a method of treating subjects suffering from diseases caused by vascular smooth muscle cell proliferation. Compounds within the scope of this disclosure effectively inhibit vascular smooth muscle cell proliferation and migration. The method comprises administering to a subject in need of treatment an amount of a compound described herein, or a pharmaceutically acceptable salt thereof, sufficient to inhibit vascular smooth muscle proliferation, and/or migration.

Another embodiment described herein is a method of treating a subject suffering from gout comprising administering to said subject in need of treatment an amount of a compound described herein, or a pharmaceutically acceptable salt thereof, sufficient to treat the condition.

Another embodiment is a method of treating a subject suffering from kidney disease, such as polycystic kidney disease, comprising administering to said subject in need of treatment an amount of a compound described herein, or a pharmaceutically acceptable salt thereof, sufficient to treat the condition.

Because of their inhibitory activity against CDKs and other kinases, the compounds described herein are also useful research tools for studying the mechanism of action of those kinases, both in vitro and in vivo.

The above-identified methods of treatment are preferably carried out by administering a therapeutically effective amount of a compound described herein (set forth below) to a subject in need of treatment. Compounds described herein are potent inhibitors of cyclin-dependent kinases. The compounds are readily synthesized and can be administered by a variety of routes, including orally and parenterally, and have little or no toxicity. The compounds described herein are members of the class of compounds described herein.

Another embodiment described herein is a pharmaceutical composition comprising a therapeutically effective amount of a compound described herein, or a pharmaceutically acceptable salt thereof, and one or more pharmaceutically acceptable excipients.

The compounds described herein are selective inhibitors of CDK4, CDK6, or CDK9, which is to say that they inhibit CDK4, CDK6, or CDK9 more potently than they inhibit tyrosine kinases and other serine-threonine kinases including other cyclin-dependent kinases such as CDK2. Despite their selectivity for CDK4, CDK6, or CDK9 inhibition, compounds described herein may inhibit other kinases, albeit at higher concentrations than those at which they inhibit CDK4, CDK6, or CDK9.

Preferred embodiments of this disclosure are compounds described herein inhibit CDK4, CDK6, or CDK9 at least about 100-fold more potently than they inhibit CDK2.

A preferred embodiment of this disclosure provides a method of inhibiting CDK4, CDK6, or CDK9 at a lower dose than is necessary to inhibit CDK2 comprising administration of a compound described herein in an effective amount that selectively inhibits CDK4, CDK6, or CDK9 over CDK2.

The compounds described herein have useful pharmaceutical and medicinal properties. Many of the compounds described herein exhibit significant selective CDK4, CDK6, or CDK9 inhibitory activity and therefore are of value in the treatment of a wide variety of clinical conditions in which CDK4, CDK6, or CDK9 kinase is abnormally elevated, or activated or present in normal amounts and activities, but where inhibition of the CDKs is desirable to treat a cellular proliferative disorder. Such disorders include, but are not limited to those enumerated in the paragraphs below.

The compounds described herein are useful for treating cancer (for example, leukemia and cancer of the lung, breast, prostate, and skin such as melanoma) and other proliferative diseases including but not limited to psoriasis, HSV, HIV, restenosis, and atherosclerosis. To utilize a compound of this disclosure to treat cancer, a patient in need of such treatment, such as one having cancer or another proliferative disease, is administered a therapeutically effective amount of a pharmaceutically acceptable composition comprising at least one compound of this disclosure.

### Methods of Diagnosis

Prior to administration of a compound described herein, a patient may be screened to determine whether a disease or condition from which the patient is or may be suffering is one which would be susceptible to treatment with a compound having activity against cyclin dependent kinases. For example, a biological sample taken from a patient may be analysed to determine whether a condition or disease, such as cancer, that the patient is or may be suffering from is one which is characterised by a genetic abnormality or abnormal protein expression which leads to over-activation of CDKs or to sensitisation of a pathway to normal CDK activity. Examples of such abnormalities that result in activation or sensitisation of the CDK2 signal include up-regulation of cyclin E or loss of p21 or p27, or presence of CDCl₄ variants. Harwell et al., J. Biol. Chem. 279(13): 12695-12705 (2004); Rajagopalan et al., Nature 428(6978): 77-81 (2004). Tumours with mutants of CDCl₄ or up-regulation, in particular over-expression, of cyclin E or loss of p21 or p27 may be particularly sensitive to CDK inhibitors. The term up-regulation includes elevated expression or over-expression, including gene amplification (i.e., multiple gene copies) and increased expression by a transcriptional effect, and hyperactivity and activation, including activation by mutations.

Thus, the patient may be subjected to a diagnostic test to detect a marker characteristic of up-regulation of cyclin E, or loss of p21 or p27, or presence of CDCl₄ variants. The term diagnosis includes screening. Markers include genetic markers such as the measurement of DNA composition to identify mutations of CDCl₄. The term marker also includes markers which are characteristic of up regulation of cyclin E, including enzyme activity, enzyme levels, enzyme state (e.g., phosphorylated or not) and mRNA levels of the aforementioned proteins. Tumours with upregulation of cyclin E, or loss of p21 or p27 may be particularly sensitive to CDK inhibitors. Tumours may preferentially be screened for upregulation of cyclin E, or loss of p21 or p27 prior to treatment. Thus, the patient may be subjected to a diagnostic test to detect a marker characteristic of up-regulation of cyclin E, or loss of p21 or p27.

The diagnostic tests are typically conducted on a biological sample selected from tumour biopsy samples, blood samples (isolation and enrichment of shed tumour cells), stool biopsies, sputum, chromosome analysis, pleural fluid, peritoneal fluid, or urine.

It has been found that there were mutations present in CDCl₄ (also known as Fbw7 or Archipelago) in human colorectal cancers and endometrial cancers. Rajagopalan et al., Nature 428(6978): 77-81 (2004); Spruck et al, Cancer Res. 62(16): 4535-4539 (2002). Identification of individual carrying a mutation in CDCl₄ may mean that the patient would be particularly suitable for treatment with a CDK inhibitor. Tumours may preferentially be screened for presence of a CDCl₄ variant prior to treatment. The screening process will typically involve direct sequencing, oligonucleotide microarray analysis, or a mutant specific antibody.

Methods of identification and analysis of mutations and up-regulation of proteins are well known to a person skilled in the art. Screening methods could include, but are not limited to, standard methods such as reverse-transcriptase polymerase chain reaction (RT-PCR) or in-situ hybridization.

In screening by RT-PCR, the level of mRNA in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel et al., Current Protocols in Molecular Biology, John Wiley & Sons Inc. (2004); Innis, M. A. et al., eds. PCR Protocols: a guide to methods and applications, Academic Press, San Diego (1990). Reactions and manipulations involving nucleic acid techniques are also described in Sambrook et al., 3^{rd} ed, Molecular Cloning: A Laboratory Manual, Cold Spring Harbor Laboratory Press (2001).

An example of an in-situ hybridization technique for assessing mRNA expression would be fluorescence in-situ hybridization (FISH). Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel et al. Current Protocols in Molecular Biology, 2004, John Wiley & Sons Inc and Fluorescence In Situ Hybridization: Technical Overview by John M. S. Bartlett in Molecular Diagnosis of Cancer, Methods and Protocols, 2^{nd} ed, 77-88 (2004).

Alternatively, the protein products expressed from the mRNAs may be assayed by immunohistochemistry of tumour samples, solid phase immunoassay with microtiter plates, Western blotting, 2-dimensional SDS-polyacrylamide gel electrophoresis, ELISA, flow cytometry and other methods known in the art for detection of specific proteins. Detection methods would include the use of site specific antibodies. The skilled person will recognize that all such well-known techniques for detection of upregulation of cyclin E, or loss of p21 or p27, or detection of CDCl₄ variants could be applicable in the present case.

Therefore, all of these techniques could also be used to identify tumours particularly suitable for treatment with the compounds described herein.

Tumours with mutants of CDCl₄ or up-regulation, in particular over-expression, of cyclin E or loss of p21 or p27 may be particularly sensitive to CDK inhibitors. Tumours may preferentially be screened for up-regulation, in particular over-expression, of cyclin E or loss of p21 or p27 or for CDCl₄ variants prior to treatment. See Harwell et al., J. Biol. Chem. 279(13): 12695-12705 (2004); Rajagopalan et al., Nature 428(6978): 77-81 (2004).

Patients with mantle cell lymphoma (MCL) could be selected for treatment with a compound described herein using diagnostic tests outlined herein. MCL is a distinct clinicopathologic entity of non-Hodgkin's lymphoma, characterized by proliferation of small to medium-sized lymphocytes with co-expression of CD5 and CD20, an aggressive and incurable clinical course, and frequent t(11;14) (q13;q32) translocation. Over-expression of cyclin D1 mRNA, found in mantle cell lymphoma (MCL), is a critical diagnostic marker. Yatabe et al., Blood 95(7): 2253-2261 (2000) proposed that cyclin D1-positivity should be included as one of the standard criteria for MCL, and that innovative therapies for this incurable disease should be explored on the basis of the new criteria. Jones et al., J. Mol. Diagn. 6(2): 84-89 (2004) developed a real-time, quantitative, reverse transcription PCR assay for cyclin D1 (CCND1) expression to aid in the diagnosis of mantle cell lymphoma (MCL). Howe et al., Clin. Chem. 50(1): 80-87 (2004) used real-time quantitative RT-PCR to evaluate cyclin D1 mRNA expression and found that quantitative RT-PCR for cyclin D1 mRNA normalized to CD 19 mRNA can be used in the diagnosis of MCL in blood, marrow, and tissue. Alternatively, patients with breast cancer could be selected for treatment with a CDK inhibitor using diagnostic tests outline above. Tumour cells commonly overexpress cyclin E and it has been shown that cyclin E is over-expressed in breast cancer. Harwell et al., Cancer Res. 60: 481-489 (2000). Breast cancer, in particular, may be treated with a CDK inhibitor as described herein.

In addition, the cancer may be analysed for INK4a and RB loss of function, and cyclin D1 or CDK4 overexpression or CDK4 mutation. RB loss and mutations inactivating p16INK4a function or hypermethylation of p16INK4a occur in many tumour types. Rb is inactivated in 100% retinoblastomas and in 90% of small cell lung carcinomas. Cyclin D1 is amplified in 40% of head and neck, over-expressed in 50% of breast cancers and 90% of mantle cell lymphomas. p16 is deleted in 60% of non-small lung carcinomas and in 40% of pancreatic cancers. CDK4 is amplified in 20% of sarcomas and in 10% of gliomas. Events resulting in RB or p16INK4a inactivation through mutation, deletion, or epigenetic silencing, or in the overexpression of cyclin D1 or Cdk4 can be identified by the techniques outlined herein. Tumours with up-regulation, in particular over-expression of cyclin D or CDK4 or loss of INK4a or RB may be particularly sensitive to CDK inhibitors. Thus, the patient may be subjected to a diagnostic test to detect a marker characteristic of over-expression of cyclin D or CDK4 or loss of INK4a or RB.

Cancers that experience INK4a and RB loss of function and cyclin D1 or CDK4 overexpression, include small cell lung cancer, non-small cell lung cancer, pancreatic cancer, breast cancer, glioblastoma multiforme, T cell ALL and mantle cell lymphoma. Therefore patients with small cell lung cancer, non-small cell lung cancer, pancreatic cancer, breast cancer, glioblastoma multiforme, T cell ALL or mantle cell lymphoma could be selected for treatment with a CDK inhibitor using diagnostic tests outlined above and may in particular be treated with a CDK inhibitor as provided herein.

Patients with specific cancers caused by aberrations in the D-Cyclin-CDK4/6-INK4-Rb pathway could be identified by using the techniques described herein and then treated with a CDK4 inhibitor as provided. Examples of abnormalities that activate or sensitise tumours to CDK4 signal include, receptor activation e.g., Her-2/Neu in breast cancer, ras mutations for example in pancreatic, colorectal or lung cancer, raf mutations for example in melanoma, p16 mutations for example in melanoma, p16 deletions for example in lung cancer, p16 methylation for example in lung cancer or cyclin D overexpression for example in breast cancer. Thus, a patient could be selected for treatment with a compound described herein using diagnostic tests as outlined herein to identify up-regulation of the D-Cyclin-CDK4/6-INK4-Rb pathway for example by overexpression of cyclin D, mutation of CDK4, mutation or depletion of pRb, deletion of p16-INK4, mutation, deletion or methylation of p16, or by activating events upstream of the CDK4/6 kinase e.g., Ras mutations or Raf mutations or hyperactive or over-expressed receptors such as Her-2/Neu.

The compounds described herein are particularly advantageous in that they are selective inhibitors of CDK4 over other cyclin dependent kinases. Compounds of this class have been previously described, but the compounds described herein have increased potency and selectivity of CDK4 over other cyclin dependent kinases. See e.g., U.S. Pat. Nos. 6,936,612; 8,685,980; 8,324,225; and U.S. Pat. Pub. No. US 20160220569.

The inhibition of protein kinase activity by the compounds described herein may be measured using a number of assays available in the art. Examples of such assays are described in the Exemplification section below.

### Pharmaceutical Compositions

The language “effective amount” of the compound is that amount necessary or sufficient to treat or prevent a protein kinase-associated disorder, e.g., prevent the various morphological and somatic symptoms of a protein kinase-associated disorder, and/or a disease or condition described herein. In an example, an effective amount of the compound described herein is the amount sufficient to treat a protein kinase-associated disorder in a subject. The effective amount can vary depending on such factors as the size and weight of the subject, the type of illness, or the particular compound described herein. For example, the choice of the compound described herein can affect what constitutes an “effective amount.” One of ordinary skill in the art would be able to study the factors contained herein and make the determination regarding the effective amount of the compounds described herein without undue experimentation.

The regimen of administration can affect what constitutes an effective amount. The compound described herein can be administered to the subject either prior to or after the onset of a protein kinase-associated disorder. Further, several divided dosages as well as staggered dosages, can be administered daily or sequentially, or the dose can be continuously infused, or can be a bolus injection. Further, the dosages of the compound(s) described herein can be proportionally increased or decreased as indicated by the exigencies of the therapeutic or prophylactic situation.

Compounds described herein may be used in the treatment of states, disorders or diseases as described herein, or for the manufacture of pharmaceutical compositions for use in the treatment of these diseases. Methods of use of the compounds described herein in the treatment of these diseases, or pharmaceutical preparations having the compounds described herein for the treatment of these diseases.

The language “pharmaceutical composition” includes preparations suitable for administration to mammals, e.g., humans. When the compounds described herein are administered as pharmaceuticals to mammals, e.g., humans, they can be given per se or as a pharmaceutical composition containing, for example, 0.1% to 99.5% (preferably, 1% to 90%) of active ingredient in combination with a pharmaceutically acceptable excipient.

The phrase “pharmaceutically acceptable excipient” includes any pharmaceutically acceptable material, composition, or vehicle, suitable for administering the compounds described herein to mammals. The excipient includes liquid or solid filler, diluent, excipient, solvent or encapsulating material, involved in carrying or transporting the subject agent from one organ, or portion of the body, to another organ, or portion of the body. Each excipient must be “acceptable” in the sense of being compatible with the other ingredients of the formulation and not injurious to the patient. Some examples of materials which can serve as pharmaceutically acceptable excipients include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers, or lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations described herein include those suitable for oral, nasal, topical, buccal, sublingual, rectal, vaginal, and/or parenteral administration. The formulations may conveniently be presented in unit dosage form and may be prepared by any methods well known in the art of pharmacy. The amount of active ingredient that can be combined with an excipient material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound as described herein with the excipient and, optionally, one or more accessory ingredients. In general, the formulations are prepared by uniformly and intimately bringing into association a compound described herein with liquid excipients, or finely divided solid excipients, or both, and then, if necessary, shaping the product.

Formulations described herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound described herein as an active ingredient. A compound described herein may also be administered as a bolus, electuary, or paste.

In solid dosage forms described herein for oral administration (capsules, tablets, pills, dragees, powders, granules and the like), the active ingredient is mixed with one or more pharmaceutically acceptable excipients, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof, and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions described herein, such as dragees, capsules, pills and granules, may optionally be scored or prepared with coatings and shells, such as enteric coatings and other coatings well known in the pharmaceutical-formulating art. They may also be formulated to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner Examples of embedding compositions that can be used include polymeric substances and waxes. The active ingredient can also be in micro-encapsulated form, if appropriate, with one or more of the above-described excipients.

Liquid dosage forms for oral administration of the compounds described herein include pharmaceutically acceptable emulsions, microemulsions, solutions, suspensions, syrups, or elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying agents, suspending agents, sweetening, flavoring, coloring, perfuming, preservative agents, or combinations thereof.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions described herein for rectal or vaginal administration may be presented as a suppository, which may be prepared by mixing one or more compounds described herein with one or more suitable nonirritating excipients or excipients comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations described herein which are suitable for vaginal administration also include pessaries, tampons, creams, gels, pastes, foams or spray formulations containing such excipients as are known in the art to be appropriate.

Dosage forms for the topical or transdermal administration of a compound described herein include powders, sprays, ointments, pastes, creams, lotions, gels, solutions, patches and inhalants. The active compound may be mixed under sterile conditions with a pharmaceutically acceptable excipient, and with any preservatives, buffers, or propellants that may be required.

The ointments, pastes, creams, and gels may contain, in addition to an active compound described herein, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound described herein, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound described herein to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this disclosure.

Pharmaceutical compositions described herein suitable for parenteral administration comprise one or more compounds described herein in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous excipients that may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, dispersing agents, or combinations thereof. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations described herein may be given orally, parenterally, topically, or rectally. They are of course given by forms suitable for each administration route. For example, they are administered in tablets or capsule form, by injection, inhalation, eye lotion, ointment, suppository, etc., administration by injection, infusion or inhalation; topical by lotion or ointment; and rectal by suppositories. Oral and/or IV administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, and intrasternal injection or infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The subject treated is typically a human subject, although it is to be understood the methods described herein are effective with respect to other animals, such as mammals and vertebrate species. More particularly, the term subject can include animals used in assays such as those used in preclinical testing including but not limited to mice, rats, monkeys, dogs, pigs and rabbits; as well as domesticated swine (pigs and hogs), ruminants, equine, poultry, felines, bovines, murines, canines, and the like.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds described herein, which may be used in a suitable hydrated form, and/or the pharmaceutical compositions described herein, are formulated into pharmaceutically acceptable dosage forms by conventional methods known to those of skill in the art.

Actual dosage levels of the active ingredients in the pharmaceutical compositions described herein may be varied to obtain an amount of the active ingredient that is effective to achieve the desired therapeutic response for a particular patient, composition, and mode of administration, without being toxic to the patient.

The selected dosage level will depend upon a variety of factors including the activity of the particular compound described herein employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In general, a suitable daily dose of a compound described herein will be that amount of the compound that is the lowest dose effective to produce a therapeutic effect. Such an effective dose will generally depend upon the factors described herein. Generally, intravenous and subcutaneous doses of the compounds described herein for a patient, when used for the indicated analgesic effects, will range from about 0.0001 to about 200 mg per kilogram of body weight per day, more preferably from about 0.01 to about 50 mg per kg per day, and still more preferably from about 1.0 to about 200 mg per kg per day. An effective amount is that amount treats a protein kinase-associated disorder.

When used in the context of a dosage amount, e.g., mg/m², the numerical weight refers to the weight of a compound described herein, exclusive of any salt, counterion, and so on. Therefore, to obtain the equivalent of 100 mg/m² of a compound described herein, it would be necessary to utilize more than 100 mg/m² of its salt, due to the additional weight of the salt.

If desired, the effective daily dose of the active compound may be administered as two, three, four, five, six or more sub-doses administered separately at appropriate intervals throughout the day, optionally, in unit dosage forms.

While it is possible for a compound described herein to be administered alone, it is preferable to administer the compound as a pharmaceutical composition.

### Synthetic Procedures

The compounds described herein are prepared from commonly available compounds using procedures known to those skilled in the art, including any one or more of the following conditions without limitation:

Within the scope of this disclosure, only a readily removable group that is not a constituent of the particular desired end product of the compounds described herein is designated a “protecting group,” unless the context indicates otherwise. The protection of functional groups by such protecting groups, the protecting groups themselves, and their cleavage reactions are described for example in standard reference works, such as e.g., Science of Synthesis: Houben-Weyl Methods of Molecular Transformation. Georg Thieme Verlag, Stuttgart, Germany (2005); McOmie, “Protective Groups in Organic Chemistry,” Plenum Press, London and New York (1973); Greene and Wuts, “Protective Groups in Organic Synthesis,” Third edition, Wiley, New York (1999); Gross and Meienhofer, eds., “The Peptides”; Vol. 3 Academic Press, London and New York (1981). A characteristic of protecting groups is that they can be removed readily (i.e., without the occurrence of undesired secondary reactions) for example by solvolysis, reduction, photolysis or alternatively under physiological conditions (e.g., by enzymatic cleavage).

Salts of the compounds described herein having at least one salt-forming group may be prepared in a manner known per se. For example, salts of the compounds described herein having acid groups may be formed, for example, by treating the compounds with metal compounds, such as alkali metal salts of suitable organic carboxylic acids, e.g., the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of the compounds described herein are obtained in customary manner, e.g., by treating the compounds with an acid or a suitable anion exchange reagent. Internal salts of the compounds described herein containing acid and basic salt-forming groups, e.g., a free carboxy group and a free amino group, may be formed, e.g., by the neutralisation of salts, such as acid addition salts, to the isoelectric point, e.g., with weak bases, or by treatment with ion exchangers.

Salts can be converted in customary manner into the free compounds; metal and ammonium salts can be converted, for example, by treatment with suitable acids, and acid addition salts, for example, by treatment with a suitable basic agent.

Mixtures of isomers obtainable as described herein can be separated in a manner known per se into the individual isomers; diastereoisomers can be separated, for example, by partitioning between polyphasic solvent mixtures, recrystallisation and/or chromatographic separation, for example over silica gel or by, e.g., medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials. Intermediates and final products can be worked up and/or purified according to standard methods, e.g., using chromatographic methods, distribution methods, (re-) crystallization, and the like.

### General Process Conditions

The following applies in general to all processes mentioned throughout this disclosure. The process steps to synthesize the compounds described herein can be carried out under reaction conditions that are known per se, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g., in the H+ form, depending on the nature of the reaction and/or of the reactants at reduced, normal or elevated temperature, for example in a temperature range of from about −100° C. to about 190° C., including, for example, from approximately −80° C. to approximately 150° C., for example at from −80° C. to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described in Science of Synthesis: Houben-Weyl Methods of Molecular Transformation. Georg Thieme Verlag, Stuttgart, Germany 2005.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of those solvents, for example aqueous solutions, unless otherwise indicated in the description of the processes. Such solvent mixtures may also be used in working up, for example by chromatography or partitioning.

The compounds, including their salts, may also be obtained in the form of hydrates, or their crystals may, for example, include the solvent used for crystallization. Different crystalline forms may be present.

Other embodiments are forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process as described herein is produced under the process conditions and processed further in situ.

### Prodrugs

Also described are pharmaceutical compositions containing, and methods of treating protein kinase-associated disorders through administering, pharmaceutically acceptable prodrugs of compounds of the compounds described herein. For example, compounds described herein having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds described herein. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews 19: 115 (1996). Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described herein, are also encompassed. Prodrugs of this type are described by TenBrink et al., J. Med. Chem., 39: 10 (1996). Free amines can also be derivatized as amides, sulfonamides, or phosphonamides. All of these prodrug moieties may incorporate groups including but not limited to ether, amine, or carboxylic acid functionalities.

Any reference to a compound described herein is therefore to be understood as referring also to the corresponding pro-drugs of the compound described herein, as appropriate and expedient.

### Combinations

The compounds described herein may also be used in combination with other agents, e.g., an additional protein kinase inhibitor that is or is not a compound described herein, for treatment of a protein kinase-associated disorder in a subject.

By the term “combination” is meant either a fixed combination in one dosage unit form, or a kit of parts for the combined administration where a compound described herein and a combination partner may be administered independently at the same time or separately within time intervals that especially allow that the combination partners show a cooperative, e.g., synergistic, effect, or any combination thereof.

The compounds described herein may be administered, simultaneously or sequentially, with an antiinflammatory, antiproliferative, chemotherapeutic agent, immunosuppressant, anti-cancer, cytotoxic agent or kinase inhibitor other than a compound described herein or salt thereof.

Further examples of agents that may be administered in combination with the compounds described herein include, but are not limited to, a PTK inhibitor, cyclosporin A, CTLA4-Ig, antibodies selected from anti-ICAM-3, anti-IL-2 receptor, anti-CD45RB, anti-CD2, anti-CD3, anti-CD4, anti-CD80, anti-CD86, and monoclonal antibody OKT3, agents blocking the interaction between CD40 and gp39, fusion proteins constructed from CD40 and gp39, inhibitors of NF-kappa B function, non-steroidal antiinflammatory drugs, steroids, gold compounds, antiproliferative agents, FK506, mycophenolate mofetil, cytotoxic drugs, TNF-α inhibitors, anti-TNF antibodies or soluble TNF receptor, rapamycin, leflunomide, cyclooxygenase-2 inhibitors, paclitaxel, cisplatin, carboplatin, doxorubicin, carminomycin, daunorubicin, aminopterin, methotrexate, methopterin, mitomycin C, ecteinascidin 743, porfiromycin, 5-fluorouracil, 6-mercaptopurine, gemcitabine, cytosine arabinoside, podophyllotoxin, etoposide, etoposide phosphate, teniposide, melphalan, vinblastine, vincristine, leurosidine, epothilone, vindesine, leurosine, or derivatives thereof.

The compound described herein and any additional agent may be formulated in separate dosage forms. Alternatively, to decrease the number of dosage forms administered to a patient, the compound described herein and any additional agent may be formulated together in any combination. For example, the compound described herein inhibitor may be formulated in one dosage form and the additional agent may be formulated together in another dosage form. Any separate dosage forms may be administered at the same time or different times.

Alternatively, a composition described herein comprises an additional agent as described herein. Each component may be present in individual compositions, combination compositions, or in a single composition.

### Chemotherapeutic Agents

As contemplated herein, the compounds described herein are orally or intravenously administered to a subject undergoing an anti-cancer therapeutic treatment regimen, for example a chemotherapeutic treatment regimen, prior to the subject receiving the anti-cancer therapy. As used herein the term “chemotherapy” or “chemotherapeutic agent” refers to treatment with a cytostatic or cytotoxic agent (i.e., a compound) to reduce or eliminate the growth or proliferation of undesirable cells, for example cancer cells. Thus, as used herein, “chemotherapy” or “chemotherapeutic agent” refers to a cytotoxic or cytostatic agent used to treat a proliferative disorder, for example cancer. The cytotoxic effect of the agent can be, but is not required to be, the result of one or more of nucleic acid intercalation or binding, DNA or RNA alkylation, inhibition of RNA or DNA synthesis, the inhibition of another nucleic acid-related activity (e.g., protein synthesis), or any other cytotoxic effect. In one embodiment, the chemotherapeutic agent is selected from etoposide, carboplatin, cisplatin, and topotecan, or a combination thereof. In one embodiment, the chemotherapeutic agent is topotecan. In one embodiment, the chemotherapeutic agent is cisplatin. In one embodiment, the chemotherapeutic agent is carboplatin. In one embodiment, the chemotherapeutic agent is etoposide.

Thus, a “cytotoxic agent” can be any one or any combination of compounds also described as “antineoplastic” agents or “chemotherapeutic agents.” Such compounds include, but are not limited to, DNA damaging compounds and other chemicals that can kill cells. “DNA damaging chemotherapeutic agents” include, but are not limited to, alkylating agents, DNA intercalators, protein synthesis inhibitors, inhibitors of DNA or RNA synthesis, DNA base analogs, topoisomerase inhibitors, and telomerase inhibitors or telomeric DNA binding compounds. For example, alkylating agents include alkyl sulfonates, such as busulfan, improsulfan, and piposulfan; aziridines, such as a benzodizepin, carboquone, meturedepa, and uredepa; ethyleneimines and methylmelamine, such as altretamine, triethylenemelamine, triethylenephosphoramide, triethylenethiophosphoramide, and trimethylolmelamine; nitrogen mustards such as chlorambucil, chlornaphazine, cyclophosphamide, estramustine, iphosphamide, mechlorethamine, mechlorethamine oxide hydrochloride, melphalan, novembichine, phenesterine, prednimustine, trofosfamide, and uracil mustard; and nitroso ureas, such as carmustine, chlorozotocin, fotemustine, lomustine, nimustine, and ranimustine.

Antibiotics used in the treatment of cancer include dactinomycin, daunorubicin, doxorubicin, idarubicin, bleomycin sulfate, mytomycin, plicamycin, and streptozocin. Chemotherapeutic antimetabolites include mercaptopurine, thioguanine, cladribine, fludarabine phosphate, fluorouracil (5-FU), floxuridine, cytarabine, pentostatin, methotrexate, and azathioprine, acyclovir, adenine β-1-D-arabinoside, amethopterin, aminopterin, 2-aminopurine, aphidicolin, 8-azaguanine, azaserine, 6-azauracil, 2′-azido-2′-deoxynucleosides, 5-bromodeoxycytidine, cytosine β-1-D-arabinoside, diazooxonorleucine, dideoxynucleosides, 5-fluorodeoxycytidine, 5-fluorodeoxyuridine, and hydroxyurea.

Chemotherapeutic protein synthesis inhibitors include abrin, aurintricarboxylic acid, chloramphenicol, colicin E3, cycloheximide, diphtheria toxin, edeine A, emetine, erythromycin, ethionine, fluoride, 5-fluorotryptophan, fusidic acid, guanylyl methylene diphosphonate and guanylyl imidodiphosphate, kanamycin, kasugamycin, kirromycin, and O-methyl threonine. Additional protein synthesis inhibitors include modeccin, neomycin, norvaline, pactamycin, paromomycine, puromycin, ricin, shiga toxin, showdomycin, sparsomycin, spectinomycin, streptomycin, tetracycline, thiostrepton, and trimethoprim. Inhibitors of DNA synthesis, include alkylating agents such as dimethyl sulfate, mitomycin C, nitrogen and sulfur mustards; intercalating agents, such as acridine dyes, actinomycins, adriamycin, anthracenes, benzopyrene, ethidium bromide, propidium diiodide-intertwining; and other agents, such as distamycin and netropsin. Topoisomerase inhibitors, such as coumermycin, nalidixic acid, novobiocin, and oxolinic acid; inhibitors of cell division, including colcemide, colchicine, vinblastine, and vincristine; and RNA synthesis inhibitors including actinomycin D, α-amanitine and other fungal amatoxins, cordycepin (3′-deoxyadenosine), dichlororibofuranosyl benzimidazole, rifampicine, streptovaricin, and streptolydigin also can be used as the DNA damaging compound.

Current chemotherapeutic agents whose toxic effects can be mitigated by the presently disclosed dosages of the compounds described herein include, but are not limited to, adriamycin, 5-fluorouracil (5FU), 6-mercaptopurine, gemcitabine, melphalan, chlorambucil, mitomycin, irinotecan, mitoxantrone, etoposide, camptothecin, topotecan, irinotecan, exatecan, lurtotecan, actinomycin-D, mitomycin, cisplatin, hydrogen peroxide, carboplatin, procarbazine, mechlorethamine, cyclophosphamide, ifosfamide, melphalan, chlorambucil, busulfan, nitrosurea, dactinomycin, daunorubicin, doxorubicin, bleomycin, plicomycin, tamoxifen, taxol, transplatinum, vinblastine, vinblastin, carmustine, cytarabine, mechlorethamine, chlorambucil, streptozocin, lomustine, temozolomide, thiotepa, altretamine, oxaliplatin, campothecin, topotecan, and methotrexate, and the like, and similar acting-type agents. In one embodiment, the DNA damaging chemotherapeutic agent is selected from the group consisting of cisplatin, carboplatin, camptothecin, doxorubicin, and etoposide. In one embodiment, the DNA damaging chemotherapeutic agent is topotecan. In one embodiment, the DNA-damaging chemotherapeutic agent is etoposide. In one embodiment, the DNA damaging chemotherapeutic agent is carboplatin. In one embodiment, the DNA damaging chemotherapeutic agent is a combination of etoposide and carboplatin.

In certain alternative embodiments, compounds described herein in a dosage described herein is also used for an anti-cancer or anti-proliferative effect in combination with a chemotherapeutic to treat a CDK4/6 replication independent, such as an Rb-negative cancer or proliferative disorder. Compounds described herein, under certain conditions, may provide an additive or synergistic effect to the chemotherapeutic, resulting in a greater anti-cancer effect than seen with the use of the chemotherapeutic alone. In one embodiment, the compounds described herein can be combined with one or more of the chemotherapeutic compounds described herein. In one embodiment, the compounds described herein can be combined with a chemotherapeutic selected from, but not limited to, tamoxifen, midazolam, letrozole, bortezomib, anastrozole, goserelin, an mTOR inhibitor, a PI3 kinase inhibitor, a dual mTOR-PI3K inhibitor, a Bruton's tyrosine kinase (BTK) inhibitor, a spleen tyrosine kinase (Syk) inhibitor, a MEK inhibitor, a RAS inhibitor, an ALK inhibitor, an HSP inhibitor (for example, an HSP70 or an HSP 90 inhibitor, or a combination thereof), a BCL-2 inhibitor, an apoptotic inducing compound, an AKT inhibitor, including but not limited to, MK-2206, GSK690693, Perifosine, (KRX-0401), GDC-0068, Triciribine, AZD5363, Honokiol, PF-04691502, and Miltefosine, a PD-1 inhibitor including but not limited to, nivolumab, CT-011, MK-3475, BMS936558, and AMP-514 or a FLT-3 inhibitor, including but not limited to, P406, dovitinib, quizartinib (AC220), amuvatinib (MP-470), tandutinib (MLN518), ENMD-2076, and KW-2449, or combinations thereof. Examples of mTOR inhibitors include but are not limited to rapamycin and its analogs, everolimus (Afinitor), temsirolimus, ridaforolimus, sirolimus, and deforolimus.

PI3k inhibitors that may be used in this disclosure are well known. Examples of PI3 kinase inhibitors include but are not limited to wortmannin, demethoxyviridin, perifosine, idelalisib, Pictilisib, Palomid 529, ZSTK474, PWT33597, CUDC-907, and AEZS-136, duvelisib, GS-9820, GDC-0032 (2-[4-[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen (S)-methylphosphonate; or Methyl(oxo) {[(2R)-1-phenoxy-2-butanyl]oxy}phosphonium)), BYL-719 ((2S)-N1-[4-Methyl-5-[2-(2,2,2-trifluoro-1,1-dimethylethyl)-4-pyridinyl]-2-thiazolyl]-1,2-pyrrolidinedicarboxamide), GSK2126458 (2,4-Difluoro-N-{2-(methyloxy)-5-[4-(4-pyridazinyl)-6-quinolinyl]-3-pyridinyl}benzenesulfonamide), TGX-221 ((±)-7-Methyl-2-(morpholin-4-yl)-9-(1-phenylaminoethyl)-pyrido[1,2-a]-pyrimidin-4-one), GSK2636771 (2-Methyl-1-(2-methyl-3-(trifluoromethyl)benzyl)-6-morpholino-1H-benzo[d]imidazole-4-carboxylic acid dihydrochloride), KIN-193 ((R)-2-((1-(7-methyl-2-morpholino-4-oxo-4H-pyrido[1,2-a]pyrimidin-9-yl)ethyl)amino)benzoic acid), TGR-1202/RP5264, GS-9820 ((S)-1-(4-((2-(2-aminopyrimidin-5-yl)-7-methyl-4-mohydroxypropan-1-one), GS-1101 (5-fluoro-3-phenyl-2-([S)]-1-[9H-purin-6-ylamino]-propyl)-3H-quinazolin-4-one), AMG-319, GSK-2269557, SAR245409 (N-(4-(N-(3-((3,5-dimethoxyphenyl)amino)quinoxalin-2-yl)sulfamoyl)phenyl)-3-methoxy-4 methylbenzamide), BAY80-6946 (2-amino-N-(7-methoxy-8-(3-morpholinopropoxy)-2,3-dihydroimidazo[1,2-c]quinaz), AS 252424 (5-[1-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-a]pyridin-6-yl)-N-tert-butylpyridine-3-sulfonamide), buparlisib (5-[2,6-Di(4-morpholinyl)-4-pyrimidinyl]-4-(trifluoromethyl)-2-pyridinamine), GDC-0941 (2-(1H-Indazol-4-yl)-6-[[4-(methylsulfonyl)-1-piperazinyl]methyl]-4-(4-morpholinyl)thieno[3,2-d]pyrimidine), GDC-0980 ((S)-1-(4-((2-(2-aminopyrimidin-5-yl)-7-methyl-4-morpholinothieno[3,2-d]pyrimidin-6yl)methyl)piperazin-1-yl)-2-hydroxypropan-1-one (also known as RG7422)), SF1126 ((8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), PF-05212384 (N-[4-[[4-(Dimethylamino)-1-piperidinyl]carbonyl]phenyl]-N′-[4-(4,6-di-4-morpholinyl-1,3,5-triazin-2-yl)phenyl]urea), LY3023414, BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile), XL-765 (N-(3-(N-(3-(3,5-dimethoxyphenylamino)quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3 aR,6E,9S,9aR,10R,11 aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5h]isochromen-10-yl]acetate (also known as sonolisib)). In one embodiment, the compounds described herein are combined in a single dosage form with the Plk3 inhibitor.

BTK inhibitors for use as described herein are well known. Examples of BTK inhibitors include ibrutinib (also known as PCI-32765)(Imbruvica™)(1-[(3R)-3-[4-amino-3-(4-phenoxy-phenyl)pyrazolo[3,4-d]pyrimidin-1-yl]piperidin-1-yl]prop-2-en-1-one), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl)acrylamide) (Avila Therapeutics) (see US Patent Publication No 20110117073), Dasatinib ([N-(2-chloro-6-methylphenyl)-2-(6-(4-(2-hydroxyethyl)piperazin-1-yl)-2-methylpyrimidin-4-ylamino)thiazole-5-carboxamide], LFM-A13 (alpha-cyano-beta-hydroxy-beta-methyl-N-(2,5-ibromophenyl) propenamide), GDC-0834 ([R—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide], CGI-560 4-(tert-butyl)-N-(3-(8-(phenylamino)imidazo[1,2-a]pyrazin-6-yl)phenyl)benzamide, CGI-1746 (4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide), CNX-774 (4-(4-((4-((3-acrylamidophenyl)amino)-5-fluoropyrimidin-2-yl)amino)phenoxy)-N-methylpicolinamide), CTA056 (7-benzyl-1-(3-(piperidin-1-yl)propyl)-2-(4-(pyridin-4-yl)phenyl)-1H-imidazo[4,5-g]quinoxalin-6(5H)-one), GDC-0834 ((R)-N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 ((R)-N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-1H-pyrazol-4-yl)benzo[h][1,6]naphthyridin-2(1H)-one), and RN486 (6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one), and other molecules capable of inhibiting BTK activity, for example those BTK inhibitors disclosed in Akinleye et al, J. Hemat. Oncol. 6: 59 (2013), the entirety of which is incorporated herein by reference. In one embodiment, the compounds described herein are combined in a single dosage form with the BTK inhibitor.

Syk inhibitors for use as described herein are well known, and include, for example, cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium(6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), RO9021 (6-[(1R,2S)-2-Amino-cyclohexylamino]-4-(5,6-dimethyl-pyridin-2-ylamino)-pyridazine-3-carboxylic acid amide), imatinib (Gleevac; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′-((5-fluoropyrimidine-2,4-diyl)bis(azanediyl))diphenol), R348 (3-Ethyl-4-methylpyridine), R406 (6-((5-fluoro-2-((3,4,5-trimethoxyphenyl)amino)pyrimidin-4-yl)amino)-2,2-dimethyl-2H-pyrido[3,2-b][1,4]oxazin-3(4H)-one), YM193306, 7-azaindole, piceatannol, ER-27319, Compound D, PRT060318, luteolin, apigenin, quercetin, fisetin, myricetin, or morin. See Singh et al. J. Med. Chem. 55: 3614-3643 (2012). In one embodiment, the compounds described herein are combined in a single dosage form with the Syk inhibitor.

MEK inhibitors for use as described herein are well known, and include, for example, tametinib/GSK1 120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl)}phenyl)acetamide), selumetinob (6-(4-bromo-2-chloroanilin)-7-fluoro-N-(2-hydroxyethoxy)-3-methylbenzimidazole-5-carboxamide), pimasertib/AS703026/MSC 1935369 ((S)-N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol), refametinib/BAY869766/RDEAI 19 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamin)-8-methylpyrido[2,3-d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6-carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2yl)methyl)benzamide), or AZD8330 (2-((2-fluoro-4-iodophenyl)amino)-N-(2 hydroxyethoxy)-1,5-dimethyl-6-oxo-1,6-dihydropyridine-3-carboxamide). In one embodiment, the compounds described herein are combined in a single dosage form with the MEK inhibitor.

Raf inhibitors for use as described herein are well known, and include, for example, Vemurafinib (N-[3-[[5-(4-Chlorophenyl)-1H-pyrrolo[2,3-b]pyridin-3-yl]carbonyl]-2,4-difluorophenyl]-1-propanesulfonamide), sorafenib tosylate (4-[4-[[4-chloro-3-(trifluoromethyl)phenyl]carbamoylamino]phenoxy]-N-methylpyridine-2-carboxamide; 4-methylbenzenesulfonate), AZ628 (3-(2-cyanopropan-2-yl)-N-(4-methyl-3-(3-methyl-4-oxo-3,4-dihydroquinazolin-6-ylamino)phenyl)benzamide), NVP-BHG712 (4-methyl-3-(1-methyl-6-(pyridin-3-yl)-1H-pyrazolo[3,4-d]pyrimidin-4-ylamino)-N-(3-(trifluoromethyl)phenyl)benzamide), RAF-265 (1-methyl-5-[2-[5-(trifluoromethyl)-1H-imidazol-2-yl]pyridin-4-yl]oxy-N-[4-(trifluoromethyl)phenyl]benzimidazol-2-amine), 2-Bromoaldisine (2-Bromo-6,7-dihydro-1H,5H-pyrrolo[2,3-c]azepine-4,8-dione), Raf Kinase Inhibitor IV (2-chloro-5-(2-phenyl-5-(pyridin-4-yl)-1H-imidazol-4-yl)phenol), and Sorafenib N-Oxide (4-[4-[[[[4-Chloro-3(trifluoroMethyl)phenyl]aMino]carbonyl]aMino]phenoxy]-N-Methyl-2pyridinecarboxaMide 1-Oxide). In one embodiment, the compounds described herein are combined in a single dosage form with the Raf inhibitor. In one embodiment, the at least one additional chemotherapeutic agent combined or alternated with the compounds described herein is a protein cell death-1 (PD-1) inhibitor. PD-1 inhibitors are known in the art, and include, for example, nivolumab (BMS), pembrolizumab (Merck), pidilizumab (CureTech/Teva), AMP-244 (Amplimmune/GSK), BMS-936559 (BMS), and MED14736 (Roche/Genentech). In one embodiment, the compounds described herein are combined in a single dosage form with the PD-1 inhibitor.

In one embodiment, the at least one additional chemotherapeutic agent combined or alternated with a selected compound disclosed herein is a B-cell lymphoma 2 (Bcl-2) protein inhibitor. BCL-2 inhibitors are known in the art, and include, for example, ABT-199 (4-[4-[[2-(4-Chlorophenyl)-4,4-dimethylcyclohex-1-en-1-yl]methyl]piperazin-1-yl]-N-[[3-nitro-4-[[(tetrahydro-2H-pyran-4-yl)methyl]amino]phenyl]sulfonyl]-2-[(1H-pyrrolo[2,3-b]pyridin-5-yl)oxy]benzamide), ABT-737 (4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide), ABT-263 ((R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3((trifluoromethyl)sulfonyl)phenyl)sulfonyl)benzamide), GX15-070 (obatoclax mesylate, (2Z)-2-[(5Z)-5-[(3,5-dimethyl-1H-pyrrol-2-yl)methylidene]-4-methoxypyrrol-2-ylidene]indole; methanesulfonic acid))), 2-methoxy-antimycin A3, YC137 (4-(4,9-dioxo-4,9-dihydronaphtho[2,3-d]thiazol-2-ylamino)-phenyl ester), pogosin, ethyl 2-amino-6-bromo-4-(1-cyano-2-ethoxy-2-oxoethyl)-4H-chromene-3-carboxylate, Nilotinib-d3, TW-37 (N-[4-[[2-(1,1-Dimethylethyl)phenyl]sulfonyl]phenyl]-2,3,4-trihydroxy-5-[[2-(1-methylethyl)phenyl]methyl]benzamide), Apogossypolone (ApoG2), or G3139 (Oblimersen). In one embodiment, the compounds described herein are administered in the dosage described herein and combined in a single dosage form with the at least one BCL-2 inhibitor.

Examples of RAS inhibitors include but are not limited to Reolysin and siG12D LODER. Examples of ALK inhibitors include but are not limited to Crizotinib, AP26113, and LDK378. HSP inhibitors include but are not limited to Geldanamycin or 17-N-Allylamino-17-demethoxygeldanamycin (17AAG), and Radicicol.

In one embodiment described herein, the compounds described herein are administered in the dosage described herein in combination with a topoisomerase inhibitor. In one aspect, an advantageous treatment of select Rb-negative cancers is disclosed using the compounds described herein a in combination with a topoisomerase inhibitor. In one embodiment, the topoisomerase inhibitor is a topoisomerase I inhibitor or a topoisomerase I and dual inhibitor. In one embodiment, the topoisomerase inhibitor is a topoisomerase II inhibitor.

In one embodiment, the topoisomerase inhibitor is selected from a topoisomerase I inhibitor. Known topoisomerase I inhibitors useful as described herein include (S)-10-[(dimethylamino)methyl]-4-ethyl-4,9-dihydroxy-1H-pyrano[3′,4′:6,7]indolizino[1,2-b]quinoline-3,14(4H,12H)-dione monohydrochloride (topotecan), (S)-4-ethyl-4-hydroxy-1H-pyrano[3′,4′:6,7]indolizino[1,2-b]quinoline-3,14-(4H,12H)-dione (camptothecin), (1 S,9 S)-1-Amino-9-ethyl-5-fluoro-1,2,3,9,12,15-hexahydro-9-hydroxy-4-methyl-10H,13H-benzo(de)pyrano(3′,4′:6,7)indolizino(1,2-b)quinoline-10,13-dione (exatecan), (7-(4-methylpiperazinomethylene)-10,11-ethylenedioxy-20(S)-camptothecin (lurtotecan), or (S)-4,11-diethyl-3,4,12,14-tetrahydro-4-hydroxy-3,14-dioxo1H-pyrano[3′,4′:6,7]-indolizino[1,2-b]quinolin-9-yl-[1,4′bipiperidine]-1′-carboxylate (irinotecan), (R)-5-ethyl-9,10-difluoro-5-hydroxy-4,5-dihydrooxepino[3′,4′:6,7]indolizino[1,2-b]quinoline-3,15(1H,13H)-dione (diflomotecan), (4S)-11-((E)-((1-Dimethylethoxy)imino)methyl)-4-ethyl-4-hydroxy-1,12-dihydro-14H-pyrano(3′,4′:6,7)indolizino(1,2-b)quinoline-3,14(4H)-dione (gimatecan), (S)-8-ethyl-8-hydroxy-15-((4-methylpiperazin-1-yl)methyl)-11,14-dihydro-2H-[1,4]dioxino[2,3-g]pyrano[3′,4′:6,7]indolizino[1,2-b]quinoline-9,12(3H,8H)-dione (lurtotecan), (4S)-4-Ethyl-4-hydroxy-11-[2-[(1-methylethyl)amino]ethyl]-1H-pyrano[3′,4′:6,7]indolizino[1,2-b]quinoline-3,14(4H,12H)-dione (belotecan), 6-((1,3-dihydroxypropan-2-yl)amino)-2,10-dihydroxy-12-((2R,3R,4S,5S,6R)-3,4,5-trihydroxy-6-(hydroxymethyl)tetrahydro-2H-pyran-2-yl)-12,13-dihydro-5H-indolo[2,3-a]pyrrolo[3,4-c]carbazole-5,7(6H)-dione (edotecarin), 8,9-dimethoxy-5-(2-N,N-dimethylaminoethyl)-2,3-methylenedioxy-5H-dibenzo(c,h)(1,6)naphthyridin-6-one (topovale), benzo[6,7]indolizino[1,2-b]quinolin-11(13H)-one (rosettacin), (S)-4-ethyl-4-hydroxy-11-(2-(trimethylsilyl)ethyl)-1H-pyrano[3′,4′:6,7]indolizino[1,2-b]quinoline-3,14(4H, 12H)-dione (cositecan), tetrakis{(4S)-9-[([1,4′-bipiperidinyl]-1′-carbonyl)oxy]-4,11-diethyl-3,14-dioxo-3,4,12,14-tetrahydro-1H-pyrano[3′,4′:6,7]indolizino[1,2-b]quinolin-4-yl}N,N′,N′,N″-{{methanetetrayltetrakis[methylenepoly(oxyethylene)oxy(1-oxoethylene)]}tetraglycinate tetrahydrochloride (etirinotecan pegol), 10-hydroxy-camptothecin (HOCPT), 9-nitrocamptothecin (rubitecan), SN38 (7-ethyl-10-hydroxycamptothecin), and 10-hydroxy-9-nitrocamptothecin (CPT109), (R)-9-chloro-5-ethyl-5-hydroxy-1 O-methyl-12-((4-methylpiperidin-1-yl)methyl)-4,5-dihydrooxepino[3′,4′:6,7]indolizino[1,2-b]quinoline-3,15(1H, 13H)-dione (elmotecan).

In a particular embodiment, the topoisomerase inhibitor is the topoisomerase I inhibitor (S)-10-[(dimethylamino)methyl]-4-ethyl-4,9-dihydroxy-1H pyrano[3′,4′:6,7]indolizino[1,2-b]quinoline-3,14(4H,12H)-dione monohydrochloride (topotecan hydrochloride). In one non-limiting example, the compounds described herein are administered in the dosage described herein in combination with a topoisomerase I inhibitor selected from the group consisting of (S)-10-[(dimethylamino)methyl]-4-ethyl-4,9-dihydroxy-1H-pyrano[3′,4′:6,7]indolizino[1,2-b]quinoline-3,14(4H,12H)-dione monohydrochloride (topotecan), (S)-4-ethyl-4-hydroxy-1H-pyrano[3′,4′:6,7]indolizino[1,2-b]quinoline-3,14-(4H, 12H)-dione (camptothecin), (1S,9S)-1-Amino-9-ethyl-5-fluoro-1,2,3,9,12,15-hexahydro-9-hydroxy-4-methyl-10H,13H-benzo(de)pyrano(3′,4′:6,7)indolizino(1,2-b)quinoline-10,13-dione (exatecan), (7-(4-methylpiperazinomethylene)-10,11-ethylenedioxy-20(S)-camptothecin (lurtotecan), or (S)-4,11-diethyl-3,4,12,14-tetrahydro-4-hydroxy-3,14-dioxo1H-pyrano[3′,4′:6,7]-indolizino[1,2-b]quinolin-9-yl-[1,4′bipiperidine]-1′-carboxylate (irinotecan). In one embodiment, the topoisomerase I inhibitor is topotecan.

### PROTACS

In addition to inhibiting the catalytic function of CDK proteins, another strategy also focuses on their level of expression. Thus the present disclosure also contemplates the use of the novel compounds described herein as Proteolysis Targeting Chimeras or “PROTACS” useful for the suppression of CDK proteins. PROTACS are bivalent inhibitors where the first moiety binds a protein targeted for ubiquitination and the second moiety for binding E3 ubiquitin ligase, thus inducing proximity that leads to ubiquitination of targets and subsequent proteolysis of the target protein in the proteasome. Thus the compounds described herein may readily be made into these chimeras for selective degradation and disruption in the cell. Thus the present disclosure contemplates bivalent compounds or PROTACs, comprising a CDK ligand (or targeting moiety) conjugated to a degradation tag.

As used herein, the term “CDK ligand” encompasses a wide variety of molecules associated with or binding to a CDK protein, including but not limited to CDK1, CDK2, CDK4, CDK6, CDK7 and CDK9. The CDK ligand may be a CDK inhibitor, capable of interfering with the enzymatic activity of the CDK proteins. CDK inhibitors include, but are not limited to, abemaciclib, palbociclib, ribociclib, trilaciclib (GIT28), GIT38 and SHR6390. As used herein, an “inhibitor” may refer to an agent that restrains, retards, or otherwise inhibits enzyme activity. Inhibitors can refer to a drug, compound, or agent that prevents or reduces the expression, transcription, or translation of a gene or protein.

### CDK-Replication Dependent Cells and Cyclin-Dependent Kinase Inhibitors

Tissue-specific stem cells and subsets of other resident proliferating cells are capable of self-renewal, meaning that they are capable of replacing themselves throughout the adult mammalian lifespan through regulated replication. Additionally, stem cells divide asymmetrically to produce “progeny” or “progenitor” cells that in turn produce various components of a given organ. For example, in the hematopoietic system, the hematopoietic stem cells give rise to progenitor cells which in turn give rise to all the differentiated components of blood (e.g., white blood cells, red blood cells, and platelets).

Certain proliferating cells, such as HSPCs, require the enzymatic activity of the proliferative kinases cyclin-dependent kinase 4 (CDK4) and/or cyclin-dependent kinase 6 (CDK6) for cellular replication. In contrast, the majority of proliferating cells in adult mammals (e.g., the more differentiated blood-forming cells in the bone marrow) do not require the activity of CDK4 and/or CDK6 (i.e., CDK4/6). These differentiated cells can proliferate in the absence of CDK4/6 activity by using other proliferative kinases, such as cyclin-dependent kinase 2 (CDK2) or cyclin-dependent kinase 1 (CDK1).

The compounds described herein show a marked selectivity for the inhibition of CDK4 and/or CDK6 in comparison to other CDKs, for example CDK2. For example, the compounds described herein provide for a dose-dependent G1-arresting effect on a subject's CDK4/6-replication dependent healthy cells, for example HSPCs or renal epithelial cells, and the methods provided for herein are sufficient to afford chemoprotection to targeted CDK4/6-replication dependent healthy cells during chemotherapeutic agent exposure, for example, during the time period that a DNA-damaging chemotherapeutic agent is capable of DNA-damaging effects on CDK4/6-replication dependent healthy cells in the subject, while allowing for the synchronous and rapid reentry into the cell-cycle by these cells shortly after the chemotherapeutic agent dissipates due to the time-limited CDK4/6 inhibitory effect provided by the compound compared to, e.g., ribociclib, palbociclib, or abemaciclib.

In some embodiments, a CDK4/6-replication dependent healthy cell is a hematopoietic stem progenitor cell. Hematopoietic stem and progenitor cells include, but are not limited to, long term hematopoietic stem cells (LT-HSCs), short term hematopoietic stem cells (ST-HSCs), multipotent progenitors (MPPs), common myeloid progenitors (CMPs), common lymphoid progenitors (CLPs), granulocyte-monocyte progenitors (GMPs), and megakaryocyte-erythroid progenitors (MEPs). Administration of the compounds described herein provides temporary, transient pharmacologic quiescence of hematopoietic stem and/or hematopoietic progenitor cells in the subject.

In some embodiments, the CDK4/6-replication dependent healthy cell may be a cell in a non-hematopoietic tissue, such as, but not limited to, the liver, kidney, pancreas, brain, lung, adrenals, intestine, gut, stomach, skin, auditory system, bone, bladder, ovaries, uterus, testicles, gallbladder, thyroid, heart, pancreatic islets, blood vessels, and the like. In some embodiments, the CDK4/6-replication dependent healthy cell is a renal cell, and in particular a renal epithelial cell, for example, a renal proximal tubule epithelial cell. In some embodiments, a CDK4/6-replication dependent healthy cell is a hematopoietic stem progenitor cell. In some embodiments, the CDK4/6-replication dependent healthy cell may be a cell in a non-hematopoietic tissue, such as, but not limited to, the liver, kidney, pancreas, brain, lung, adrenals, intestine, gut, stomach, skin, auditory system, bone, bladder, ovaries, uterus, testicles, gallbladder, thyroid, heart, pancreatic islets, blood vessels, and the like.

Likewise, the compounds described herein provides for a dose-dependent mitigating effect on CDK4/6-replication dependent healthy cells that have been exposed to toxic levels of chemotherapeutic agents, allowing for repair of DNA damage associated with chemotherapeutic agent exposure and synchronous, rapid reentry into the cell-cycle following dissipation of the CDK4/6 inhibitory effect compared to, e.g., ribociclib, palbociclib, or abemaciclib. In one embodiment, the use of the compounds described herein results in the G1-arresting effect on the subject's CDK4/6-replication dependent healthy cells dissipating following administration of the compounds described herein so that the subject's healthy cells return to or approach their pre-administration baseline cell-cycle activity within less than about 24 hours, 30 hours, 36 hours, or 40 hours, of administration. In one embodiment, the G1-arresting effect dissipates such that the subject's CDK4/6-replication dependent healthy cells return to their pre-administration baseline cell-cycle activity within less than about 24 hours, 30 hours, 36 hours, or 40 hours.

In one embodiment, the use of the compounds described herein results in the G1-arresting effect dissipating such that the subject's CDK4/6-dependent healthy cells return to or approach their pre-administration baseline cell-cycle activity within less than about 24 hours, 30 hours, 36 hours, or 40 hours of the chemotherapeutic agent effect. In one embodiment, the G1-arresting effect dissipates such that the subject's CDK4/6-replication dependent cells return to their pre-administration baseline cell-cycle activity within less than about 24 hours, 30 hours, 36 hours, or 40 hours, or within about 48 hours of the cessation of the chemotherapeutic agent administration. In one embodiment, the CDK4/6-replication dependent healthy cells are HSPCs. In one embodiment, the CDK4/6-dependent healthy cells are renal epithelial cells.

In one embodiment, the use of the compounds described herein results in the G1-arresting effect dissipating so that the subject's CDK4/6-replication dependent healthy cells return to or approach their pre-administration baseline cell-cycle activity within less than about 24 hours, 30 hours, 36 hours, 40 hours, or within less than about 48 hours from the point in which the CDK4/6 inhibitor's concentration level in the subject's blood drops below a therapeutic effective concentration.

In one embodiment, the compounds described herein are used to protect renal epithelium cells during exposure to a chemotherapeutic agent, for example, a DNA damaging chemotherapeutic agent, wherein the renal epithelial cells are transiently prevented from entering S-phase in response to chemotherapeutic agent induced renal tubular epithelium damage for no more than about 24 hours, about 30 hours, about 36 hours, about 40 hours, or about 48 hours from the point in which the compounds described herein concentration level in the subject's blood drops below a therapeutic effective concentration or biological effective concentration, from the cessation of the chemotherapeutic agent effect, or from administration of the compounds described herein.

The compounds described herein may be synchronous in their off-effect, that is, upon dissipation of the G1 arresting effect, CDK4/6-replication dependent healthy cells exposed to the compounds described herein in the concentrations described herein reenter the cell-cycle in a similarly timed fashion. CDK4/6-replication dependent healthy cells that reenter the cell-cycle do so such that the normal proportion of cells in G1 and S are reestablished quickly and efficiently, within less than about 24 hours, 30 hours, 36 hours, 40 hours, or within about 48 hours of the from the point in which the compounds described herein concentration level in the subject's blood drops below a therapeutic effective concentration. This advantageously allows for a larger number of healthy cells to begin replicating upon dissipation of the G1 arrest compared with asynchronous CDK4/6 inhibitors such as ribociclib, palbociclib, or abemaciclib.

In addition, synchronous cell-cycle reentry following G1 arrest using the compounds described herein in concentrations and dosages described herein provides for the ability to time the administration of hematopoietic growth factors to assist in the reconstitution of hematopoietic cell lines to maximize the growth factor effect. In one embodiment described herein, the compounds described herein can be administered in a concerted regimen with a blood growth factor agent. As such, in one embodiment, the use of the compounds described herein and methods described herein are combined with the use of hematopoietic growth factors including, but not limited to, granulocyte colony stimulating factor (G-CSF, for example, sold as Neupogen (filgrastin), Neulasta (peg-filgrastin), or lenograstin), granulocyte-macrophage colony stimulating factor (GM-CSF, for example sold as molgramostim and sargramostim (Leukine)), M-CSF (macrophage colony stimulating factor), thrombopoietin (megakaryocyte growth development factor (MGDF), for example sold as Romiplostim and Eltrombopag) interleukin (IL)-12, interleukin-3, interleukin-11 (adipogenesis inhibiting factor or oprelvekin), SCF (stem cell factor, steel factor, kit-ligand, or KL) and erythropoietin (EPO), and their derivatives (sold as for example epoetin-α as Darbopoetin, Epocept, Nanokine, Epofit, Epogin, Eprex and Procrit; epoetin-β sold as for example NeoRecormon, Recormon and Micera), epoetin-delta (sold as for example Dynepo), epoetin-omega (sold as for example Epomax), epoetin zeta (sold as for example Silapo and Reacrit) as well as for example Epocept, EPOTrust, Erypro Safe, Repoeitin, Vintor, Epofit, Erykine, Wepox, Espogen, Relipoeitin, Shanpoietin, Zyrop and EPIAO). In one embodiment, the compounds described herein are administered prior to administration of the hematopoietic growth factor. In one embodiment, the hematopoietic growth factor administration is timed so that the compounds described herein inhibitory effect on HSPCs has dissipated.

### CDK 4/6-Replication Independent Proliferative Disorders

One aspect described herein is a dosing regimen comprising the administration of the compounds described herein that provides a specific PK and/or PD blood profile followed by the administration of a chemotherapeutic agent for the treatment of the CDK 4/6-replication independent cellular proliferation disorder. The subject treated as described herein may be undergoing therapeutic chemotherapy for the treatment of a proliferative disorder that is CDK4/6 replication independent.

CDK 4/6-replication independent cellular proliferation disorders, for example as seen in certain types of cancer, can be characterized by one or a combination of increased activity of cyclin-dependent kinase 1 (CDK1), increased activity of cyclin-dependent kinase 2 (CDK2), loss, deficiency, or absence of retinoblastoma tumor suppressor protein (Rb)(Rb-null), high levels of MYC expression, increased cyclin E1, E2, and increased cyclin A. The cancer may be characterized by reduced expression of the retinoblastoma tumor suppressor protein or a retinoblastoma family member protein or proteins (such as, but not limited to p107 and p130). In one embodiment, the subject is undergoing chemotherapeutic treatment for the treatment of an Rb-null or Rb-deficient cancer, including but not limited to small cell lung cancer, triple-negative breast cancer, HPV-positive head and neck cancer, retinoblastoma, Rb-negative bladder cancer, Rb negative prostate cancer, osteosarcoma, or cervical cancer. Administration of the compounds described herein may allow for a higher dose of a chemotherapeutic agent to be used to treat the disease than the standard dose that would be safely used in the absence of administration of the compounds described herein.

The host or subject, including a human, may be undergoing chemotherapeutic treatment of a non-malignant proliferative disorder, or other abnormal cellular proliferation, such as a tumor, multiple sclerosis, lupus, or arthritis.

Proliferative disorders that are treated with chemotherapy include cancerous and non-cancer diseases. In a typical embodiment, the proliferative disorder is a CDK4/6-replication independent disorder. The compounds described herein are effective in protecting healthy CDK4/6-replication dependent cells, for example HSPCs, during chemotherapeutic treatment of a broad range of tumor types, including but not limited to the following: breast, prostate, ovarian, skin, lung, colorectal, brain (i.e., glioma) and renal. Preferably, the compounds described herein should not compromise the efficacy of the chemotherapeutic agent or arrest G1 arrest the cancer cells. Many cancers do not depend on the activities of CDK4/6 for proliferation as they can use the proliferative kinases promiscuously (e.g., can use CDK 1/2/4/or 6) or lack the function of the retinoblastoma tumor suppressor protein (Rb), which is inactivated by the CDKs. The potential sensitivity of certain tumors to CDK4/6 inhibition can be deduced based on tumor type and molecular genetics using standard techniques. Cancers that are not typically affected by the inhibition of CDK4/6 are those that can be characterized by one or more of the group including, but not limited to, increased activity of CDK1 or CDK2, loss, deficiency, or absence of retinoblastoma tumor suppressor protein (Rb), high levels of MYC expression, increased cyclin E (e.g., E1 or E2) and increased cyclin A, or expression of a Rb-inactivating protein (such as HPV-encoded E7). Such cancers can include, but are not limited to, small cell lung cancer, retinoblastoma, HPV positive malignancies like cervical cancer and certain head and neck cancers, MYC amplified tumors such as Burkitts' Lymphoma, and triple negative breast cancer; certain classes of sarcoma, certain classes of non-small cell lung carcinoma, certain classes of melanoma, certain classes of pancreatic cancer, certain classes of leukemia, certain classes of lymphoma, certain classes of brain cancer, certain classes of colon cancer, certain classes of prostate cancer, certain classes of ovarian cancer, certain classes of uterine cancer, certain classes of thyroid and other endocrine tissue cancers, certain classes of salivary cancers, certain classes of thymic carcinomas, certain classes of kidney cancers, certain classes of bladder cancers, and certain classes of testicular cancers.

The loss or absence of retinoblastoma (Rb) tumor suppressor protein (Rb-null) can be determined through any of the standard assays known to one of ordinary skill in the art, including but not limited to Western Blot, ELISA (enzyme linked immunoadsorbent assay), IHC (immunohistochemistry), and FACS (fluorescent activated cell sorting). The selection of the assay will depend upon the tissue, cell line or surrogate tissue sample that is utilized e.g., for example Western Blot and ELISA may be used with any or all types of tissues, cell lines or surrogate tissues, whereas the IHC method would be more appropriate wherein the tissue utilized in the methods described herein was a tumor biopsy. FACs analysis would be most applicable to samples that were single cell suspensions such as cell lines and isolated peripheral blood mononuclear cells. See e.g., US 20070212736.

Alternatively, molecular genetic testing may be used for determination of retinoblastoma gene status. Molecular genetic testing for retinoblastoma includes the following as described in Lohmann and Gallie “Retinoblastoma. Gene Reviews” (2010) or Parsam et al., J. Genetics 88(4): 517-527 (2009).

Increased activity of CDK1 or CDK2, high levels of MYC expression, increased cyclin E and increased cyclin A can be determined through any of the standard assays known to one of ordinary skill in the art, including but not limited to Western Blot, ELISA (enzyme linked immunoadsorbent assay), IHC (immunohistochemistry), and FACS (fluorescent activated cell sorting). The selection of the assay will depend upon the tissue, cell line, or surrogate tissue sample that is utilized e.g., for example Western Blot and ELISA may be used with any or all types of tissues, cell lines, or surrogate tissues, whereas the IHC method would be more appropriate wherein the tissue utilized in the methods described herein was a tumor biopsy. FACs analysis would be most applicable to samples that were single cell suspensions such as cell lines and isolated peripheral blood mononuclear cells.

In some embodiments, the cancer is selected from a small cell lung cancer, retinoblastoma, and triple negative (ER/PR/Her2 negative) or “basal-like” breast cancer, which almost always have inactivate retinoblastoma tumor suppressor proteins (Rb), and therefore do not require CDK4/6 activity to proliferate. Triple negative (basal-like) breast cancer is also almost always genetically or functionally Rb-null. Also, certain virally induced cancers (e.g., cervical cancer and subsets of Head and Neck cancer) express a viral protein (E7) which inactivates Rb making these tumors functionally Rb-null. Some lung cancers are also believed to be caused by HPV. In one particular embodiment, the cancer is small cell lung cancer, and the patient is treated with a DNA-damaging agent selected from the group consisting of etoposide, carboplatin, and cisplatin, or a combination thereof.

The compounds described herein can also be used in protecting healthy CDK4/6-replication dependent cells during chemotherapeutic treatments of abnormal tissues in non-cancer proliferative diseases, including but not limited to: psoriasis, lupus, arthritis (notably rheumatoid arthritis), hemangiomatosis in infants, multiple sclerosis, myelodegenerative disease, neurofibromatosis, ganglioneuromatosis, keloid formation, Paget's Disease of the bone, fibrocystic disease of the breast, Peyronie's and Duputren's fibrosis, restenosis, and cirrhosis. Further, the compounds described herein can be used to ameliorate the effects of chemotherapeutic agents in the event of accidental exposure or overdose (e.g., methotrexate overdose).

### Chemoprotective Regimes

In certain embodiments, the compounds described herein or pharmaceutically acceptable compositions, salts, isotopic analogs, or prodrugs thereof, are administered at a dose described herein so that the protection afforded by the compound is short term and transient in nature, allowing a significant portion of the cells to synchronously renter the cell-cycle quickly following the cessation of the chemotherapeutic agent's effect, for example within less than about 24, 30, 36, or 40 hours. Cells that are quiescent within the G1 phase of the cell cycle are more resistant to the damaging effect of chemotherapeutic agents than proliferating cells.

The compounds described herein can be administered to the subject prior to treatment with a chemotherapeutic agent, during treatment with a chemotherapeutic agent, after exposure to a chemotherapeutic agent, or a combination thereof. The compounds described herein are typically administered in a manner that allows the drug facile access to the blood stream, for example via intravenous injection. In one embodiment, the compound is administered to the subject less than about 24 hours, 20 hours, 16 hours, 12 hours, 8 hours, or 4 hours, 2.5 hours, 2 hours, 1 hour, 1/2 hour or less prior to treatment with the chemotherapeutic agent. In an alternative embodiment, the compound is administered to the subject less than about 48 hours, 40 hours, 36 hours, or 32 hours or less prior to treatment with the chemotherapeutic agent.

Typically, the compound described herein is administered to the subject prior to treatment with the chemotherapeutic agent such that the compound reaches peak serum levels before or during treatment with the chemotherapeutic agent. In one embodiment, are administered to the subject about 30 minutes prior to administration of the chemotherapeutic agent. In one embodiment, the compounds described herein are administered to the subject over about a 30 minute period and then the subject is administered a chemotherapeutic agent. In one embodiment, the Compound is administered concomitantly, or closely thereto, with the chemotherapeutic agent exposure. If desired, the compound can be administered multiple times during the chemotherapeutic agent treatment to maximize inhibition, especially when the chemotherapeutic drug is administered over a long period or has a long half-life. In an alternative embodiment, the compounds described herein can be administered following exposure to the chemotherapeutic agent if desired to mitigate healthy cell damage associated with chemotherapeutic agent exposure. In certain embodiments, the compound is administered up to about ½ hour, up to about 1 hour, up to about 2 hours, up to about 4 hours, up to about 8 hours, Up to about 10 hours, up to about 12 hours, up to about 14 hours, up to about 16 hours, or up to about 20 hours or greater following the chemotherapeutic agent exposure. In a particular embodiment, the compound is administered up to between about 12 hours and 20 hours following exposure to the chemotherapeutic agent.

Importantly, at the dosing ranges provided herein, the compounds described herein can be used in a multi-day chemotherapeutic regimen without concomitant accumulation in the subject. Accordingly, the PK and/or PD levels provided herein are not significantly altered, that is, by no more than about 10%, across a multi-day dosing regimen. Because of this, the compounds described herein are ideal chemoprotectants in chemotherapeutic treatment regimens that require multi-day chemotherapeutic agent administration, for example as seen in small cell lung cancer, triple negative breast cancer, bladder cancer, and HPV-positive head and neck and cervical cancer.

In one aspect, the use of the compounds described herein at the PK and PD parameters described herein allows for a chemo-protective regimen for use during standard chemotherapeutic dosing schedules or regimens common in many anti-cancer treatments. For example, the compounds described herein can be administered so that CDK4/6-replication dependent healthy cells are G1 arrested during chemotherapeutic agent exposure wherein, due to the rapid dissipation of the G1-arresting effect of the compounds, a significant number of healthy cells reenter the cell-cycle and are capable of replicating shortly after chemotherapeutic agent exposure, for example, within less than about 24, 30, 40, or 48 hours, and continue to replicate until administration of the compounds described herein in anticipation of the next chemotherapeutic treatment. In one embodiment, the compounds described herein are administered to allow for the cycling of the CDK4/6-replication dependent healthy cells between G1-arrest and reentry into the cell-cycle to accommodate a repeated-dosing chemotherapeutic treatment regimen, for example including but not limited to a treatment regimen wherein the chemotherapeutic agent is administered: on day 1-3 every 21 days; on days 1-3 every 28 days; on day 1 every 3 weeks; on day 1, day 8, and day 15 every 28 days, on day 1 and day 8 every 28 days; on days 1 and 8 every 21 days; on days 1-5 every 21 days; 1 day a week for 6-8 weeks; on days 1, 22, and 43; days 1 and 2 weekly; days 1-4 and 22-25; 1-4; 22-25, and 43-46; and similar type-regimens, wherein the CDK4/6-replication dependent cells are G1 arrested during chemotherapeutic agent exposure. In one embodiment, the compounds described herein can be administered so that the subject's CDK4/6-replication dependent cells are G1-arrested during daily chemotherapeutic agent exposure, for example a contiguous multi-day chemotherapeutic regimen. In one embodiment, the compounds described herein can be administered so that the subject's CDK4/6-replication dependent cells are G1-arrested during chemotherapeutic agent exposure, for example a contiguous multi-day regimen, but a significant portion of healthy cells reenter the cell-cycle and replicate during the off periods before starting the next cycle of chemotherapeutic agent exposure, for example cycle 2, cycle 3, cycle 4, etc. In one embodiment, the compounds described herein are administered so that a subject's CDK4/6-replication dependent cells' G1-arrest is provided during a daily chemotherapeutic agent treatment regimen, for example, a contiguous multi-day treatment regimen, and the arrested cells are capable of reentering the cell-cycle shortly after the multi-day regimen ends.

In one embodiment, the subject has small cell lung cancer and the compounds described herein are administered intravenously over about a 30 minute period about 30 minutes prior to administration of either etoposide or carboplatin on day 1, and etoposide on days 2 and 3 during a 21-day treatment cycle, wherein the subject is administered both etoposide and carboplatin on day 1 and etoposide on day 2 and 3 during a 21-day cycle first line treatment protocol. In one embodiment, the dose of etoposide administered is 100 mg/m² administered intravenously over about 60 minutes daily on days 1, 2, and 3 of each 21-day cycle. In one embodiment, the dose of carboplatin administered to the subject is calculated using the Calvert formula with a target AUC of 5 (maximum dose of 750 mg) administered intravenously over 30 minutes on day 1 of each 21-day cycle.

In one embodiment, the subject has small cell lung cancer and the compounds described herein are administered intravenously over about a 30 minute period about 30 minutes prior to administration of topotecan during a 21-day treatment cycle, wherein the subject is administered topotecan on days 1, 2, 3, 4, and 5 during a 21-day cycle second or third line treatment protocol. In one embodiment, the dose of topotecan administered is 1.5 mg/m² administered intravenously over about 30 minutes daily on days 1, 2, 3, 4, and 5 of each 21-day cycle. In one embodiment, the dose of topotecan administered is 1.25 mg/m² administered intravenously over about 30 minutes daily on days 1, 2, 3, 4, and 5 of each 21-day cycle. In one embodiment, the dose of topotecan administered is 0.75 mg/m² administered intravenously over about 30 minutes daily on days 1, 2, 3, 4, and 5 of each 21-day cycle.

In one embodiment, the subject has small cell lung cancer and the compounds described herein are administered intravenously over about a 30 minute period about 30 minutes prior to administration of topotecan during a 21-day treatment cycle, wherein the subject is administered topotecan on days 1, 2, and 3 during a 21-day cycle second or third line treatment protocol. In one embodiment, the dose of topotecan administered is 1.25 mg/m² administered intravenously over about 30 minutes daily on days 1, 2, and 3 of each 21-day cycle.

Administration of the compounds described herein in the doses described herein can result in reduced anemia, reduced lymphopenia, reduced thrombocytopenia, or reduced neutropenia compared to that typically expected after, common after, or associated with treatment with chemotherapeutic agents in the absence of administration of the compounds described herein. The use of the compounds described herein results in a faster recovery from bone marrow suppression associated with long-term use of CDK4/6 inhibitors, such as myelosuppression, anemia, lymphopenia, thrombocytopenia, or neutropenia, following the cessation of use of the compounds described herein. In some embodiments, the use of the compounds described herein results in reduced or limited bone marrow suppression associated with long-term use of CDK4/6 inhibitors, such as myelosuppression, anemia, lymphopenia, thrombocytopenia, or neutropenia.

In one embodiment, the compounds described herein, at the concentrations and doses described herein, is used in a CDK4/6-replication dependent healthy cell cycling strategy wherein a subject is exposed to regular, repeated chemotherapeutic treatments, wherein the healthy cells are G1-arrested when chemotherapeutic agent exposed and allowed to reenter the cell-cycle before the subject's next chemotherapeutic treatment. Such cycling allows CDK4/6-replication dependent cells to regenerate damaged blood cell lineages between regular, repeated treatments, for example those associated with standard chemotherapeutic treatments for cancer, and reduces the risk associated with long term CDK4/6 inhibition. This cycling between a state of G1-arrest and a state of replication is not feasible in limited time-spaced, repeated chemotherapeutic agent exposures using longer acting CDK4/6 inhibitors such as ribociclib, palbociclib, or abemaciclib, as the lingering G1-arresting effects of the compound prohibit significant and meaningful reentry into the cell-cycle before the next chemotherapeutic agent exposure or delay the healthy cells from entering the cell cycle and reconstituting damaged tissues or cells following treatment cessation.

The compounds described herein can be administered to a subject on any chemotherapeutic treatment schedule and in any dose consistent with the prescribed course of treatment. The compounds described herein can be administered prior to, during, or following the administration of the chemotherapeutic agent. In one embodiment, the compounds described herein can be administered to the subject during the time period ranging from 24 hours prior to chemotherapeutic treatment until 24 hours following exposure. This time period, however, can be extended to time earlier that 24 hour prior to exposure to the agent (e.g., based upon the time it takes the chemotherapeutic agent used to achieve suitable plasma concentrations and/or the compound's plasma half-life). Further, the time period can be extended longer than 24 hours following exposure to the chemotherapeutic agent so long as later administration of the compounds described herein leads to at least some protective effect. Such post-exposure treatment can be especially useful in cases of accidental exposure or overdose. In an alternative embodiment, the compounds described herein can be administered to the subject during the time period ranging from 48 hours prior to chemotherapeutic treatment until 48 hours following exposure.

In some embodiments, the compounds described herein can be administered to the subject at a time period prior to the administration of the chemotherapeutic agent, so that plasma levels of the compounds described herein are peaking at the time of administration of the chemotherapeutic agent. If convenient, the compounds described herein can be administered at the same time as the chemotherapeutic agent, in order to simplify the treatment regimen. In some embodiments, the chemoprotectant and chemotherapeutic can be provided in a single formulation.

In some embodiments, the compounds described herein can be administered to the subject such that the chemotherapeutic agent can be administered either at higher doses (increased chemotherapeutic dose intensity) or more frequently (increased chemotherapeutic dose density) or at a dose that achieves equivalent AUC therapeutic levels as seen when the chemotherapeutic agent is administered alone.

Dose-dense chemotherapy is a chemotherapy treatment plan in which drugs are given with less time between treatments than in a standard chemotherapy treatment plan. Chemotherapy dose intensity represents unit dose of chemotherapy administered per unit time. Dose intensity can be increased or decreased through altering dose administered, time interval of administration, or both. Myelosuppression continues to represent the major dose-limiting toxicity of cancer chemotherapy, resulting in considerable morbidity and mortality along with frequent reductions in chemotherapy dose intensity, which may compromise disease control and survival. The compounds and their use as described herein represent a way of increasing chemotherapy dose density and/or dose intensity while mitigating adverse events such as, but not limited to, myelosuppression.

If desired, multiple doses of the compounds described herein can be administered to the subject. Alternatively, the subject can be given a single dose of the compounds described herein. For example, the compounds described herein can be administered so that CDK4/6-replication dependent healthy cells are G1 arrested during chemotherapeutic agent exposure wherein, due to the rapid dissipation of the G1-arresting effect of the compounds, a significant number of healthy cells reenter the cell-cycle and are capable of replicating shortly after chemotherapeutic agent exposure, for example, within about 24-48 hours or less, and continue to replicate until administration of the CDK4/6-inhibitor in anticipation of the next chemotherapeutic treatment. In one embodiment, the compounds described herein are administered to allow for the cycling of the CDK4/6-replication dependent healthy cells between G1-arrest and reentry into the cell-cycle to accommodate a repeated-dosing chemotherapeutic treatment regimen, for example, including but not limited to a treatment regimen wherein the chemotherapeutic agent is administered: on day 1-3 every 21 days; on days 1-3 every 28 days; on day 1 every 3 weeks; on day 1, day 8, and day 15 every 28 days, on day 1 and day 8 every 28 days; on days 1 and 8 every 21 days; on days 1-5 every 21 days; 1 day a week for 6-8 weeks; on days 1, 22, and 43; days 1 and 2 weekly; days 1-4 and 22-25; 1-4; 22-25, and 43-46; and similar type-regimens, wherein the CDK4/6-replication dependent cells are G1 arrested during chemotherapeutic agent exposure and a significant portion of the cells reenter the cell-cycle in between chemotherapeutic agent exposure.

As contemplated herein, the compounds described herein can be used as a chemoprotectant in conjunction with a number of standard of care chemotherapeutic treatment regimens used to provide chemoprotection to a subject's CDK4/6-replication dependent healthy cells during a CDK4/6-replication independent cancer treatment protocol. In alternative embodiments, for example, the compounds described herein can be administered to provide chemoprotection in a small cell lung cancer therapy protocol such as, but not limited to: cisplatin 60 mg/m² IV on day 1 plus etoposide 120 mg/m² IV on days 1-3 every 21 d for 4 cycles; cisplatin 80 mg/m² IV on day 1 plus etoposide 100 mg/m² IV on days 1-3 every 28 d for 4 cycles; cisplatin 60-80 mg/m² IV on day 1 plus etoposide 80-120 mg/m² IV on days 1-3 every 21-28 d (maximum of 4 cycles); carboplatin AUC 5-6 min-mg/mL IV on day 1 plus etoposide 80-100 mg/m² IV on days 1-3 every 28 d (maximum of 4 cycles); Cisplatin 60-80 mg/m² IV on day 1 plus etoposide 80-120 mg/m² IV on days 1-3 every 21-28 d; carboplatin AUC 5-6 min-mg/mL IV on day 1 plus etoposide 80-100 mg/m² IV on days 1-3 every 28 d (maximum 6 cycles); cisplatin 60 mg/m² IV on day 1 plus irinotecan 60 mg/m² IV on days 1, 8, and 15 every 28 d (maximum 6 cycles); cisplatin 30 mg/m² IV on days 1 and 8 or 80 mg/m² IV on day 1 plus irinotecan 65 mg/m² IV on days 1 and 8 every 21 d (maximum 6 cycles); carboplatin AUC 5 min-mg/mL IV on day 1 plus irinotecan 50 mg/m² IV on days 1, 8, and 15 every 28 d (maximum 6 cycles); carboplatin AUC 4-5 IV on day 1 plus irinotecan 150-200 mg/m² IV on day 1 every 21 d (maximum 6 cycles); cyclophosphamide 800-1000 mg/m² IV on day 1 plus doxorubicin 40-50 mg/m² IV on day 1 plus vincristine 1-1.4 mg/m² IV on day 1 every 21-28 d (maximum 6 cycles); Etoposide 50 mg/m² P0 daily for 3 wk every 4 wk; topotecan 2.3 mg/m² PO on days 1-5 every 21 d; topotecan 1.5 mg/m² IV on days 1-5 every 21 d; carboplatin AUC 5 min-mg/mL IV on day 1 plus irinotecan 50 mg/m² IV on days 1, 8, and 15 every 28 d; carboplatin AUC 4-5 IV on day 1 plus irinotecan 150-200 mg/m² IV on day 1 every 21 d; cisplatin 30 mg/m² IV on days 1, 8, and 15 plus irinotecan 60 mg/m² IV on days 1, 8, and 15 every 28 d; cisplatin 60 mg/m² IV on day 1 plus irinotecan 60 mg/m² IV on days 1, 8, and 15 every 28 d; cisplatin 30 mg/m² IV on days 1 and 8 or 80 mg/m² IV on day 1 plus irinotecan 65 mg/m² IV on days 1 and 8 every 21 d; paclitaxel 80 mg/m² IV weekly for 6 wk every 8 wk; paclitaxel 175 mg/m² IV on day 1 every 3 wk; etoposide 50 mg/m² PO daily for 3 wk every 4 wk; topotecan 2.3 mg/m² PO on days 1-5 every 21 d; topotecan 1.5 mg/m² IV on days 1-5 every 21 d; carboplatin AUC 5 min-mg/mL IV on day 1 plus irinotecan 50 mg/m² IV on days 1, 8, and 15 every 28 d; carboplatin AUC 4-5 IV on day 1 plus irinotecan 150-200 mg/m² IV on day 1 every 21 d; cisplatin 30 mg/m² IV on days 1, 8, and 15 plus irinotecan 60 mg/m² IV on days 1, 8, and 15 every 28 d; cisplatin 60 mg/m² IV on day 1 plus irinotecan 60 mg/m² IV on days 1, 8, and 15 every 28 d; cisplatin 30 mg/m² IV on days 1 and 8 or 80 mg/m² IV on day 1 plus irinotecan 65 mg/m² IV on days 1 and 8 every 21 d; paclitaxel 80 mg/m² IV weekly for 6 wk every 8 wk; and paclitaxel 175 mg/m² IV on day 1 every 3 wk. In alternative embodiments, the compounds described herein are administered to provide chemoprotection in a small cell lung cancer therapy protocol such as, but not limited to: topotecan 2.0 mg/m² PO on days 1-5 every 21 d; topotecan 1.5-2.3 mg/m² PO on days 1-5 every 21 d; etoposide 100 mg/m² intravenously (IV) on days 1 through 3 plus cisplatin 50 mg/m² IV on days 1 and 2 (treatment cycles administered every 3 weeks to a maximum of six cycles); etoposide 100 mg/m² intravenously (IV) on days 1 through 3 plus carboplatin 300 mg/m² IV on day 1 (treatment cycles administered every 3 weeks to a maximum of six cycles); carboplatin (300 mg/m² IV on day 1) and escalating doses of etoposide starting with 80 mg/m² IV on days 1-3; carboplatin 125 mg/m²/day combined with etoposide 200 mg/m²/day administered for 3 days; etoposide 80-200 mg/m² intravenously (IV) on days 1 through 3 plus carboplatin 125-450 mg/m² IV on day 1 (treatment cycles administered every 21-28 days); carboplatin AUC 5-6 min-mg/mL IV on day 1 plus etoposide 80-200 mg/m² IV on days 1-3 every 28 d (maximum of 4 cycles).

In one embodiment, the compounds described herein are administered in a dosage describe herein to a subject with small cell lung cancer on days 1, 2, and 3 of a treatment protocol wherein the DNA damaging agent selected from the group consisting of carboplatin, etoposide, and cisplatin, or a combination thereof, is administered on days 1, 2, and 3 every 21 days.

In one embodiment, the compounds described herein are used to provide chemoprotection to a subject's CDK4/6-replication dependent healthy cells during a CDK4/6-replication independent head and neck cancer treatment protocol. In one embodiment, the compounds described herein are administered to provide chemoprotection in a CDK4/6-replication independent head and neck cancer therapy protocol such as, but not limited to: cisplatin 100 mg/m² IV on days 1, 22, and 43 or 40-50 mg/m² IV weekly for 6-7 wk; cetuximab 400 mg/m² IV loading dose 1 wk before the start of radiation therapy, then 250 mg/m² weekly (premedicate with dexamethasone, diphenhydramine, and ranitidine); cisplatin 20 mg/m² IV on day 2 weekly for up to 7 wk plus paclitaxel 30 mg/m² IV on day 1 weekly for up to 7 wk; cisplatin 20 mg/m²/day IV on days 1-4 and 22-25 plus 5-FU 1000 mg/m²/day by continuous IV infusion on days 1-4 and 22-25; 5-FU 800 mg/m² by continuous IV infusion on days 1-5 given on the days of radiation plus hydroxyurea 1 g PO q12 h (11 doses per cycle); chemotherapy and radiation given every other week for a total of 13 wk; carboplatin 70 mg/m²/day IV on days 1-4, 22-25, and 43-46 plus 5-FU 600 mg/m²/day by continuous IV infusion on days 1-4, 22-25, and 43-46; carboplatin AUC 1.5 IV on day 1 weekly plus paclitaxel 45 mg/m² IV on day 1 weekly; cisplatin 100 mg/m² IV on days 1, 22, and 43 or 40-50 mg/m² IV weekly for 6-7 wk; docetaxel 75 mg/m² IV on day 1 plus cisplatin 100 mg/m² IV on day 1 plus 5-FU 100 mg/m²/day by continuous IV infusion on days 1-4 every 3 wk for 3 cycles, then 3-8 wk later, carboplatin AUC 1.5 IV weekly for up to 7 wk during radiation therapy; docetaxel 75 mg/m² IV on day 1 plus cisplatin 75 mg/m² IV on day 1 plus 5-FU 750 mg/m²/day by continuous IV infusion on days 1-4 every 3 wk for 4 cycles; cisplatin 100 mg/m² IV on day 1 every 3 wk for 6 cycles plus 5-FU 1000 mg/m²/day by continuous IV infusion on days 1-4 every 3 wk for 6 cycles plus cetuximab 400 mg/m² IV loading dose on day 1, then 250 mg/m² IV weekly until disease progression (premedicate with dexamethasone, diphenhydramine, and ranitidine); carboplatin AUC 5 min-mg/mL IV on day 1 every 3 wk for 6 cycles plus 5-FU 1000 mg/m²/day by continuous IV infusion on days 1-4 every 3 wk for 6 cycles plus cetuximab 400 mg/m² IV loading dose on day 1, then 250 mg/m² IV weekly until disease progression (premedicate with dexamethasone, diphenhydramine, and ranitidine); cisplatin 75 mg/m² IV on day 1 plus docetaxel 75 mg/m² IV on day 1 every 3 wk; cisplatin 75 mg/m² IV on day 1 plus paclitaxel 175 mg/m² IV on day 1 every 3 wk; carboplatin AUC 6 IV on day 1 plus docetaxel 65 mg/m² IV on day 1 every 3 wk; carboplatin AUC 6 IV on day 1 plus paclitaxel 200 mg/m² IV on day 1 every 3 wk; cisplatin 75-100 mg/m² IV on day 1 every 3-4 wk plus cetuximab 400 mg/m² IV loading dose on day 1, then 250 mg/m² IV weekly (premedicate with dexamethasone, diphenhydramine, and ranitidine); cisplatin 100 mg/m² IV on day 1 plus 5-FU 1000 mg/m²/day by continuous IV infusion on days 1-4 every 3 wk; methotrexate 40 mg/m² IV weekly (3 wk equals 1 cycle); paclitaxel 200 mg/m² IV every 3 wk; docetaxel 75 mg/m² IV every 3 wk; cetuximab 400 mg/m² IV loading dose on day 1, then 250 mg/m² IV weekly until disease progression (premedicate with dexamethasone, diphenhydramine, and ranitidine); cisplatin 100 mg/m² IV on day 1 every 3 wk for 6 cycles plus 5-FU 1000 mg/m²/day by continuous IV infusion on days 1-4 every 3 wk for 6 cycles plus cetuximab 400 mg/m² IV loading dose on day 1, then 250 mg/m² IV weekly (premedicate with dexamethasone, diphenhydramine, and ranitidine); carboplatin AUC 5 min-mg/mL IV on day 1 every 3 wk for 6 cycles plus 5-FU 1000 mg/m²/day by continuous IV infusion on days 1-4 every 3 wk for 6 cycles plus cetuximab 400 mg/m² IV loading dose on day 1, then 250 mg/m² IV weekly (premedicate with dexamethasone, diphenhydramine, and ranitidine); cisplatin 75 mg/m² IV on day 1 plus docetaxel 75 mg/m² IV on day 1 every 3 wk; cisplatin 75 mg/m² IV on day 1 plus paclitaxel 175 mg/m² IV on day 1 every 3 wk; carboplatin AUC 6 IV on day 1 plus docetaxel 65 mg/m² IV on day 1 every 3 wk; carboplatin AUC 6 IV on day 1 plus paclitaxel 200 mg/m² IV on day 1 every 3 wk; cisplatin 75-100 mg/m² IV on day 1 every 3-4 wk plus cetuximab 400 mg/m² IV loading dose on day 1, then 250 mg/m² IV weekly (premedicate with dexamethasone, diphenhydramine, and ranitidine); cisplatin 100 mg/m² IV on day 1 plus 5-FU 1000 mg/m²/day by continuous IV infusion on days 1-4 every 3 wk; methotrexate 40 mg/m² IV weekly (3 wk equals 1 cycle); paclitaxel 200 mg/m² IV every 3 wk; docetaxel 75 mg/m² IV every 3 wk; cetuximab 400 mg/m² IV loading dose on day 1, then 250 mg/m² IV weekly until disease progression (premedicate with dexamethasone, diphenhydramine, and ranitidine); cisplatin 100 mg/m² IV on days 1, 22, and 43 with radiation, then cisplatin 80 mg/m² IV on day 1 plus 5-FU 1000 mg/m²/day by continuous IV infusion on days 1-4 every 4 wk for 3 cycles; cisplatin 75 mg/m² IV on day 1 plus docetaxel 75 mg/m² IV on day 1 every 3 wk; cisplatin 75 mg/m² IV on day 1 plus paclitaxel 175 mg/m² IV on day 1 every 3 wk; carboplatin AUC 6 IV on day 1 plus docetaxel 65 mg/m² IV on day 1 every 3 wk; carboplatin AUC 6 IV on day 1 plus paclitaxel 200 mg/m² IV on day 1 every 3 wk; cisplatin 100 mg/m² IV on day 1 plus 5-FU 1000 mg/m²/day by continuous IV infusion on days 1-4 every 3 wk; cisplatin 50-70 mg/m² IV on day 1 plus gemcitabine 1000 mg/m² IV on days 1, 8, and 15 every 4 wk; gemcitabine 1000 mg/m² IV on days 1, 8, and 15 every 4 wk or gemcitabine 1250 mg/m² IV on days 1 and 8 every 3 wk; methotrexate 40 mg/m² IV weekly (3 wk equals 1 cycle); paclitaxel 200 mg/m² IV every 3 wk; docetaxel 75 mg/m² IV every 3 wk; cisplatin 75 mg/m² IV on day 1 plus docetaxel 75 mg/m² IV on day 1 every 3 wk; cisplatin 75 mg/m² IV on day 1 plus paclitaxel 175 mg/m² IV on day 1 every 3 wk; carboplatin AUC 6 IV on day 1 plus docetaxel 65 mg/m² IV on day 1 every 3 wk; carboplatin AUC 6 IV on day 1 plus paclitaxel 200 mg/m² IV on day 1 every 3 wk; cisplatin 100 mg/m² IV on day 1 plus 5-FU 1000 mg/m²/day by continuous IV infusion on days 1-4 every 3 wk; cisplatin 50-70 mg/m² IV on day 1 plus gemcitabine 1000 mg/m² IV on days 1, 8, and 15 every 4 wk; gemcitabine 1000 mg/m² IV on days 1, 8, and 15 every 4 wk or gemcitabine 1250 mg/m² IV on days 1 and 8 every 3 wk; methotrexate 40 mg/m² IV weekly (3 wk equals 1 cycle); paclitaxel 200 mg/m² IV every 3 wk; and docetaxel 75 mg/m² IV every 3 wk.

In one embodiment, the compounds described herein are used to provide chemoprotection to a subject's CDK4/6-replication dependent healthy cells during a CDK4/6-replication independent triple negative breast cancer treatment protocol. In one embodiment, the compounds described herein are administered to provide a blood plasma concentration described herein to provide chemoprotection in a CDK4/6-replication independent triple negative breast cancer therapy protocol such as, but not limited to: dose-dense doxorubicin (adriamycin) and cyclophosphamide (cytoxan) every two weeks for four cycles followed by dose-dense paclitaxel (Taxol) every two weeks for four cycles; adriamycin/paclitaxel/cyclophosphomide every three weeks for a total of four cycles; adriamycin/paclitaxel/cyclophosphomide every two weeks for a total of four cycles; adriamycin/cyclophosphomide followed by paclitaxel (Taxol) every three weeks for four cycles each; and adriamycin/cyclophosphomide followed by paclitaxel (Taxol) every two weeks for four cycles each.

In one embodiment, the compounds described herein are used to provide chemoprotection to a subject's CDK4/6-replication dependent healthy cells during a CDK4/6-replication independent bladder cancer treatment protocol. In one embodiment, the compounds described herein are administered to provide a blood plasma concentration described herein to provide chemoprotection in a CDK4/6-replication independent bladder cancer therapy protocol such as, but not limited to: postoperative adjuvant intravesical chemotherapy for non-muscle invasive bladder cancer, first-line chemotherapy for muscle-invasive bladder cancer, and second-line chemotherapy for muscle invasive bladder cancer. Non-limiting examples of postoperative chemotherapy for bladder cancer include one dose or mitomycin (40 mg), epirubicin (80 mg), thiotepa (30 mg), or doxorubicin (50 mg). Non-limiting examples of first-line chemotherapy for bladder cancer include: gemcitabine 1000 mg/m² on days 1, 8, and 15 plus cisplatin 70 mg/m² on day 1 or 2 repeating cycle every 28 days for a total of four cycles; dosing methotrexate 30 mg/m² IV on days 1, 15, and 22 plus vinblastine 3 mg/m² IV on days 2, 15, and 22 plus doxorubicin 30 mg/m² IV on day 2 plus cisplatin 70 mg/m² IV on day 2, repeat cycle every 28 d for a total of 3 cycles; and dose-dense regimens of the above administered along with doses of growth factor stimulants.

In one embodiment, the compounds described herein are used to provide chemoprotection to a subject's CDK4/6-replication dependent healthy cells during a CDK4/6-replication independent retinoblastoma treatment protocol. In one embodiment, the compounds described herein are administered to provide a blood plasma concentration described herein to provide chemoprotection in a CDK4/6-replication independent retinoblastoma therapy protocol such as, but not limited to the administration of carboplatin, vincristine, or etoposide in conjunction with surgery, radiotherapy, cryotherapy, thermotherapy, or other local therapy techniques.

In one embodiment, the compounds described herein are used to provide chemoprotection to a subject's CDK4/6-replication dependent healthy cells during a CDK4/6-replication independent cervical cancer treatment protocol. In one embodiment, the compounds described herein are administered to provide a blood plasma concentration described herein to provide chemoprotection in a CDK4/6-replication independent cervical cancer therapy protocol such as, but not limited to the administration of cisplatin 40 mg/m² IV once weekly, cisplatin 50-75 mg/m² IV on day 1 plus 5-fluorouracil (5-FU) 1000 mg/m² continuous IV infusion on days 2-5 and days 30-33, cisplatin 50-75 mg/m² IV on day 1 plus 5-FU 1000 mg/m² IV infusion over 24 hour on days 1-4 every 3 weeks for 3-4 cycles, bevacizumab 15 mg/kg IV over 30-90 minutes plus cisplatin on day 1 or 2 plus paclitaxel on day 1 every 3 weeks, bevacizumab plus paclitaxel on day 1 plus topotecan on days 1-3 every 3 weeks, paclitaxel followed by cisplatin on day 1 every 3 weeks, topotecan on days 1-3 followed by cisplatin on day 1 every 3 weeks, and paclitaxel on day 1 every 3 weeks. In another embodiment, the cervical cancer therapy protocol is as above in addition to radiation, surgery, or another procedure.

Triple-negative breast cancer (TNBC) is defined as the absence of staining for estrogen receptor, progesterone receptor, and HER2/neu. TNBC is insensitive to some of the most effective therapies available for breast cancer treatment including HER2-directed therapy such as trastuzumab and endocrine therapies such as tamoxifen or the aromatase inhibitors. Combination cytotoxic chemotherapy administered in a dose-dense or metronomic schedule remains the standard therapy for early-stage TNBC. Platinum agents have recently emerged as drugs of interest for the treatment of TNBC with carboplatin added to paclitaxel and adriamycin plus cyclophosphamide chemotherapy in the neoadjuvant setting. The poly (ADP-ribose) polymerase (PARP) inhibitors are emerging as promising therapeutics for the treatment of TNBC. PARPs are a family of enzymes involved in multiple cellular processes, including DNA repair.

As a nonlimiting illustration, the subject is exposed to chemotherapeutic agent at least 5 times a week, at least 4 times a week, at least 3 times a week, at least 2 times a week, at least 1 time a week, at least 3 times a month, at least 2 times a month, or at least 1 time a month, wherein the subject's CDK4/6-replication dependent healthy cells are G1 arrested during treatment and allowed to cycle in between chemotherapeutic agent exposure, for example during a treatment break. In one embodiment, the subject is undergoing 5 times a week chemotherapeutic treatment, wherein the subject's CDK4/6-replication dependent healthy cells are G1 arrested during the chemotherapeutic agent exposure and allowed to reenter the cell-cycle during the 2 day break, for example, over the weekend.

In one embodiment, using the compounds described herein at the dosage described herein, the subject's CDK4/6-replication dependent healthy cells are arrested during the entirety of the chemotherapeutic agent exposure time-period, for example, during a contiguous multi-day regimens, the cells are arrested over the time period that is required to complete the contiguous multi-day course, and then allowed to recycle at the end of the contiguous multi-day course. In one embodiment, using the compounds described herein at the dosage described herein, the subject's CDK4/6-replication dependent healthy cells are arrested during the entirety of the chemotherapeutic regimen, for example, in a daily chemotherapeutic exposure for three weeks, and rapidly reenter the cell-cycle following the completion of the therapeutic regimen.

In one embodiment, the subject has been exposed to a chemotherapeutic agent, and, using the compounds described herein at the dosage described herein, the subject's CDK4/6-replication dependent healthy cells are placed in G1 arrest following exposure in order to mitigate, for example, DNA damage. In one embodiment, the compounds described herein at the dosage described herein is administered at least ½ hour, at least 1 hour, at least 2 hours, at least 3 hours, at least 4 hours, at least 5 hours, at least 6 hours, at least 7 hours, at least 8 hours, at least 10 hours, at least 12 hours, at least 14 hours, at least 16 hours, at least 18 hours, at least 20 hours or more post chemotherapeutic agent exposure.

In some embodiments, the CDK4/6-replication dependent healthy cells can be arrested for longer periods to allow for intensified chemotherapeutic treatment, for example, over a period of hours, days, and/or weeks, through multiple, time separated administrations of a CDK4/6 inhibitor described herein. Because of the rapid and synchronous reentry into the cell cycle by CDK4/6-replication dependent healthy cells, for example HSPCs, upon dissipation of the CDK4/6 inhibitors intra-cellular effects, the cells are capable of reconstituting the cell lineages faster than CDK4/6 inhibitors with longer G1 arresting profiles, for example, ribociclib, palbociclib, or abemaciclib.

The reduction in chemotoxicity afforded by the compounds described herein at the dosage described herein can allow for dose intensification (e.g., more therapy can be given in a fixed period of time) in medically related chemotherapies, which will translate to better efficacy. Therefore, the presently disclosed methods can result in chemotherapy regimens that are less toxic and more effective.

The use of the compounds described herein at the dosage described herein can induce selective G1 arrest in CDK4/6-dependent cells (e.g., as measured in a cell-based in vitro assay).

In one embodiment, the compounds described herein at the dosage described herein is capable of increasing the percentage of CDK4/6-dependent cells in the G1 phase, while decreasing the percentage of CDK4/6-dependent cells in the G2/M phase and S phase. In one embodiment, the compounds described herein at the dosage described herein induces substantially pure (i.e., “clean”) G1 cell cycle arrest in the CDK4/6-dependent cells (e.g., wherein treatment with the compounds described herein induces cell cycle arrest such that the majority of cells are arrested in G1 as defined by standard methods (e.g., propidium iodide (PI) staining or others) with the population of cells in the G2/M and S phases combined being less than about 30%, about 25%, about 20%, about 15%, about 10%, about 5%, about 3% or less of the total cell population.

Methods of assessing the cell phase of a population of cells are known in the art (see, for example, in U.S. Patent Application Publication No. 20020224522) and include cytometric analysis, microscopic analysis, gradient centrifugation, elutriation, fluorescence techniques including immunofluorescence, and combinations thereof. Cytometric techniques include exposing the cell to a labeling agent or stain, such as DNA-binding dyes, e.g., PI, and analyzing cellular DNA content by flow cytometry. Immunofluorescence techniques include detection of specific cell cycle indicators such as, for example, thymidine analogs (e.g., 5-bromo-2-deoxyuridine (BrdU) or an iododeoxyuridine), with fluorescent antibodies.

In some embodiments, the use of the compounds described herein at the dosage described herein results in reduced or substantially free off-target effects, particularly related to inhibition of kinases other than CDK4 and or CDK6 such as CDK2, as the compounds described herein at the dosage described herein is a poor inhibitor (e.g., >1 μM IC₅₀) of CDK2. Furthermore, because of the high selectivity for CDK4/6, the use of the compounds described herein should not induce cell cycle arrest in CDK4/6-independent cells. In addition, because of the short transient nature of the G1-arrest effect, the CDK4/6-replication dependent cells more quickly reenter the cell-cycle than, comparatively, use of palbociclib provides, resulting in the reduced risk of, in one embodiment, hematological toxicity development during long term treatment regimens due to the ability of HSPCs to replicate between chemotherapeutic treatments.

In some embodiments, the use of the compounds described herein at the dosage described herein reduces the risk of undesirable off-target effects including, but not limited to, long term toxicity, anti-oxidant effects, and estrogenic effects. Anti-oxidant effects can be determined by standard assays known in the art. For example, a compound with no significant anti-oxidant effects is a compound that does not significantly scavenge free-radicals, such as oxygen radicals. The anti-oxidant effects of a compound can be compared to a compound with known anti-oxidant activity, such as genistein. Thus, a compound with no significant anti-oxidant activity can be one that has less than about 2, 3, 5, 10, 30, or 100-fold anti-oxidant activity relative to genistein. Estrogenic activities can also be determined via known assays. For instance, a non-estrogenic compound is one that does not significantly bind and activate the estrogen receptor. A compound that is substantially free of estrogenic effects can be one that has less than about 2, 3, 5, 10, 20, or 100-fold estrogenic activity relative to a compound with estrogenic activity, e.g., genistein.

### Dosing Profiles

One embodiment described herein is the particular dosing and blood profile ranges of the CDK4/6 inhibitor compounds described herein, and methods using said dosages, for treating a subject undergoing DNA-damaging chemotherapeutic therapy for the treatment of a CDK 4/6-replication independent cellular proliferation disorder.

As contemplated herein and for purposes of the disclosed ranges herein, all ranges described herein include any and all numerical values occurring within the identified ranges. For example, a range of 1 to 10, as contemplated herein, would include the numerical values 1, 2, 3, 4, 5, 6, 7, 8, 9, 10, as well as fractions thereof.

The term “AUC” (amount-time/volume) as used herein means the area under the plasma concentration-time curve.

The term “AUC_{inf}” (amount-time/volume) as used herein means the area under the plasma concentration-time curve from time zero to infinity.

The term “AUCₜ” (amount-time/volume) as used herein means the area under the plasma concentration time curve from time zero to time t.

The term “AUC_{τ},” (amount-time/volume) as used herein means the area under the plasma concentration-time curve during a dosage interval (τ).

The term “AUCₗₐₛₜ” (amount-time/volume) as used herein means the area under the plasma concentration-time curve from time zero to time of the last measurable concentration.

The term “Cₘₐₓ” (amount/volume) as used herein means the maximum (peak) plasma drug concentration.

The term “CL” (volume/time or volume/time/kg) as used herein means the apparent total body clearance of the drug from plasma.

The term “CL/F” (volume/time or volume/time/kg) as used herein means the apparent total clearance of the drug from plasma after administration.

The term “κ” (time⁻¹) as used herein means first-order rate constant.

The term “κ₁₂” (time⁻¹) as used herein means the transfer rate constant (first-order) from the central (1) to the peripheral (2) compartment.

The term “κ₂₁” (time⁻¹) as used herein means the transfer rate constant (first-order) from the peripheral (2) to the central (1) compartment.

The term “κ₃₁” (time⁻¹) as used herein means the transfer rate constant (first-order) from the deep peripheral (3) to the central (1) compartment.

The term “κ_{Z}” (time⁻¹) as used herein means the terminal disposition rate constant/terminal rate constant.

The term “MRT_{inf}” (time) as used herein means mean residence time.

The term “Tₘₐₓ” (time) as used herein means time to reach maximum (peak) plasma concentration following drug administration.

The term “T½” (time) as used herein means the elimination half-life as used in one or non-compartmental models.

The term “T½β” (time) as used herein means the terminal elimination half-life as used in two-compartmental models.

The term “T½γ” (time) as used herein means the terminal or elimination half-life as used in three compartmental models.

The term “Vₛₛ” (volume or volume/kg) as used herein means the apparent volume of distribution at steady state.

In one embodiment, the compounds described herein are orally or intravenously administered to a subject prior to administration of a chemotherapeutic agent so that a single dose of the compounds described herein provides a specific PK and/or PD blood profile as described herein. In one embodiment, the dose administered to the subject is between about 180 and about 215 mg/m². In one embodiment, the dose is between about 180 and about 280 mg/m². In one embodiment, the dose administered is between about 170 to about 215 mg/m². In one embodiment, the dose administered is between about 170 to about 280 mg/m². For example, the dose is about 170, 175, 180, 185, 190, 195, 200, 205, 210, 215, 220, 225, 230, 235, 240, 245, 250, 255, 260, 265, 270, 275, or 280 mg/m². In one embodiment, the dose is about 190 mg/m². In one embodiment, the dose is about 200 mg/m². In one embodiment, the dose is about 240 mg/m². In one embodiment, the dose administered provides for a mean AUCₗₐₛₜ measured at 24.5 hours or a mean Cₘₐₓ as described below.

In another embodiment, the compounds described herein are orally or intravenously administered to a subject prior to administration of a chemotherapeutic agent so that a single dose of the compounds described herein provides a blood plasma level profile dosage-corrected mean Cₘₐₓ ((ng/mL)/(mg/m²)) of between about 1 (ng/mL)/(mg/m²) and 20 (ng/mL)/(mg/m²), between about 2.5 (ng/mL)/(mg/m²) and 15 (ng/mL)/(mg/m²), or of between about 4 (ng/mL)/(mg/m²) and 12 (ng/mL)/(mg/m²). In one embodiment, the dosage-corrected mean Cₘₐₓ((ng/mL)/(mg/m²) is about 1, 2, 3, 4, 5, 6, 7, 8, 9, 10, 11, 12, 13, 14, 15, 16, 17, 18, 19, or 20 ((ng/mL)/(mg/m²). In one embodiment, the dosage-corrected mean Cₘₐₓ ((ng/mL)/(mg/m²)) is about 8.0 (ng/mL)/(mg/m²)±3.5 (ng/mL)/(mg/m²), about 8.5 (ng/mL)/(mg/m²)±2.5 (ng/mL)/(mg/m²), about 9.5 (ng/mL)/(mg/m²)±2.0 (ng/mL)/(mg/m²), or about 10.2 (ng/mL)/(mg/m²)±1.5 (ng/mL)/(mg/m²). In one embodiment, the dosage-corrected mean Cₘₐₓ is about 6.0±20%. The dosage corrected mean Cₘₐₓ is mean Cₘₐₓ divided by the number of milligrams/m² of the compounds described herein in the formulation. In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m².

Another embodiment is a method of treating a subject undergoing chemotherapy for the treatment of an CDK 4/6-replication independent cellular proliferation disorder by providing an intravenously administered formulation of the compounds described herein wherein a single-dose provides a blood plasma level profile dosage-corrected mean Cₘₐₓ ((ng/mL)/(mg/m²)) of between about 4.6 (ng/mL)/(mg/m²) and about 17.1 (ng/mL)/(mg/m²) or about 1.8 (ng/mL)/(mg/m²) to about 16.8 (ng/mL)/(mg/m²). In one embodiment, the dosage-corrected mean Cₘₐₓ ((ng/mL)/(mg/m²)) is about at least 8.5 (ng/mL)/(mg/m²) or about at least 3.8 (ng/mL)/(mg/m²).

In another embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a dosage-corrected mean Cₘₐₓ (ng/mL)/(mg/m²) of between of between about 1 (ng/mL)/(mg/m²) and 20 (ng/mL)/(mg/m²), between about 2.5 (ng/mL)/(mg/m²) and 15 (ng/mL)/(mg/m²), or of between about 4 (ng/mL)/(mg/m²) and 14 (ng/mL)/(mg/m²). In one embodiment, the dosage-corrected mean Cₘₐₓ ((ng/mL)/(mg/m²) is about 1, 2, 3, 4, 5, 6, 7, 8, 9, 10, 11, 12, 13, 14, 15, 16, 17, 18, 19, or 20 ((ng/mL)/(mg/m²). In one embodiment, the dosage-corrected mean Cₘₐₓ ((ng/mL)/(mg/m²)) is about 9.5 (ng/mL)/(mg/m²)±1.5 (ng/mL)/(mg/m²). In an alternative embodiment the dosage-corrected mean Cₘₐₓ is about 9.5 (ng/mL)/(mg/m²)±1.9 (ng/mL)/(mg/m²) or 9.5 (ng/mL)/(mg/m²)±about 20%. In one embodiment, the mean dose-corrected Cₘₐₓ ((ng/mL)/(mg/m²)) is about 10.45 (ng/mL)/(mg/m²)±about 20%. In one embodiment, the dosage-corrected mean Cₘₐₓ is about 6.0 ((ng/mL)/(mg/m²))±20%. In one embodiment, the dosage-corrected mean Cₘₐₓ is about 6.5 ((ng/mL)/(mg/m²))±20%. In one embodiment, the compounds described herein are administered on days 1 and 2 of the treatment regime. In one embodiment, the compounds described herein are administered on days 1, 2, and 3 of the treatment regime. In one embodiment, the compounds described herein are administered on days 1, 2, 3, and 4 of the treatment regime. In one embodiment, the compounds described herein are administered on days 1, 2, 3, 4, and 5 of the treatment regime.

In another embodiment, the compounds described herein are orally or intravenously administered to a subject prior to administration of a chemotherapeutic agent so that a single dose of the compounds described herein provides a blood plasma level profile dosage-corrected with a mean Cₘₐₓ (ng/mL) of between about 1000 ng/mL and about 3500 ng/mL, or between about 1400 ng/mL and about 3100 ng/mL, or between about 1700 ng/mL and about 2500 ng/mL, or between about 1900 ng/mL and about 2150 ng/mL. In one embodiment, the mean Cₘₐₓ (ng/mL) is about 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, or 3500 (ng/mL). In one embodiment, the mean Cₘₐₓ (ng/mL) is about 2030 ng/mL±555 ng/mL. In one embodiment, the mean Cₘₐₓ (ng/mL) is about 1900 ng/mL, about 1950 ng/mL, about 1975 ng/mL, about 2000 ng/mL, about 2025 ng/mL, about 2030 ng/mL, about 2040 ng/mL, about 2050 ng/mL, about 2075 ng/mL, or about 2100 ng/mL. In one embodiment, the maximum mean concentration occurs at the end of the infusion period of the formulation. In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m².

In another embodiment, the compounds described herein are orally or intravenously administered to a subject prior to administration of a chemotherapeutic agent so that a single dose of the compounds described herein provides a blood plasma level profile dosage-corrected a mean Cₘₐₓ (ng/mL) of between about 885 ng/mL and about 3280 ng/mL, or between about 355 ng/mL and about 3360 ng/mL. In one embodiment, the mean Cₘₐₓ (ng/mL) is about at least 1705 ng/mL. In one embodiment, the Cₘₐₓ (ng/mL) is about at least 752 ng/mL.

In another embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a mean Cₘₐₓ (ng/mL) of between about 1000 ng/mL and 3500 ng/mL. In one embodiment, the mean Cₘₐₓ (ng/mL) is between about 1400 ng/mL and about 3100 ng/mL. In one embodiment, the mean Cₘₐₓ (ng/mL) of about 1000, 1100, 1200, 1300, 1400, 1450, 1500, 1550, 1600, 1650, 1700, 1750, 1800, 1850, 1900, 1950, 2000, 2050, 2100, 2150, 2200, 2250, 2300, 2400, 2450, 2500, 2600, 2650, 2700, 2750, 2800, 2850, 2900, 2950, 3000, 3050, 3100, 3200, 3300, 3400, 3500 (ng/mL). In one embodiment, the mean Cₘₐₓ (ng/mL) is about 2030 ng/mL±555 ng/mL. In an alternative embodiment, the mean Cₘₐₓ is about 2030 ng/mL±406 ng/mL or about 2030 ng/mL about 20%. In an alternative embodiment, the mean Cₘₐₓ is about 2230 ng/mL±about 20%. In one embodiment, the mean Cₘₐₓ is at least about 1020 ng/mL. In one embodiment, the maximum mean concentration occurs at the end of the infusion period of the compounds described herein. In one embodiment, the compounds described herein are administered on days 1 and 2 of the treatment regime. In one embodiment, the compounds described herein are administered on days 1, 2, and 3 of the treatment regime. In one embodiment, the compounds described herein are administered on days 1, 2, 3, and 4 of the treatment regime. In one embodiment, the compounds described herein are administered on days 1, 2, 3, 4, and 5 of the treatment regime.

In another embodiment, provided is a method of treating a subject undergoing chemotherapy for the treatment of a CDK 4/6-replication independent cellular proliferation disorder by providing an intravenous administered formulation of the compounds described herein wherein a single-dose provides a blood plasma level profile with a mean Tₘₐₓ (h) of between about 0.10 hrs and about 1.0 hrs, of between about 0.20 hrs and about 0.6 hrs, or of between about 0.30 hrs and about 0.5 hrs. In one embodiment, the Tₘₐₓ (h) is about 0.10, 0.15, 0.20, 0.25, 0.30, 0.35, 0.40, 0.45, 0.50, 0.55, 0.60, 0.65, 0.70, 0.85, 0.90, 0.95, or 1.0 (h). In one embodiment, the mean Tₘₐₓ (h) is about 0.417 hrs 0.129 hrs. In one embodiment, the mean Tₘₐₓ (h) is about 0.3 hrs, about 0.35 hrs, about 0.375 hrs, about 0.40 hrs, about 0.415 hrs, about 0.425 hrs, about 0.45 hrs, about 0.475 hrs, or about 0.5 hrs. In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a Tₘₐₓ (h) as described herein.

Another embodiment is a method of treating a subject undergoing chemotherapy for the treatment of a CDK 4/6-replication independent cellular proliferation disorder by providing an intravenous administered formulation of the compounds described herein wherein a single-dose provides a blood plasma level profile with a mean Tₘₐₓ (h) of between about 0.25 hrs and about 0.48 hrs. In one embodiment, the mean Tₘₐₓ (h) is about at least 0.47 hrs.

In another embodiment, the compounds described herein are orally or intravenously administered to a subject prior to administration of a chemotherapeutic agent so that a single dose of the compounds described herein provides a blood plasma level profile mean AUC_{inf}(h·ng/mL) measured over 24.5 hours after administration of between about 2000 h·ng/mL to about 4500 h·ng/mL, of between about 2300 h·ng/mL to about 4000 h·ng/mL, of between about 2500 h·ng/mL to about 3500 h·ng/mL, or of between about 2700 h·ng/mL to about 3200 h·ng/mL. In one embodiment, the mean AUC_{inf} (h·ng/mL) is about 2000, 2100, 2200, 2300, 2400, 2500, 2600, 2700, 2800, 2900, 3000, 3100, 3200, 3300, 3400, 3500, 3600, 3700, 3800, 3900, or 4000 (h·ng/mL). In one embodiment, the mean AUC_{inf} (h·ng/mL) measured over 24.5 hours after administration is about 3050 h·ng/mL±513 h·ng/mL. In one embodiment, the mean AUC_{inf}(h·ng/mL) measured over 24.5 hours after administration is about 2500 h·ng/mL, is about 2750 h·ng/mL, about 2900 h·ng/mL, about 3000 h·ng/mL, about 3050 h·ng/mL, about 3100 h·ng/mL, about 3250 h·ng/mL, about 3300 h·ng/mL. In one embodiment, the mean AUC_{inf} (h·ng/mL) measured over 72.5 hours after administration is between about 2000 h·ng/mL to about 4500 h·ng/mL, of between about 2300 h·ng/mL to about 4000 h·ng/mL, of between about 2500 h·ng/mL to about 3500 h·ng/mL, or of between about 2700 h·ng/mL to about 3200 h·ng/mL. In one embodiment, the mean AUC_{inf} (h·ng/mL) measured over 72.5 hours after administration is about 2000, 2100, 2200, 2300, 2400, 2500, 2600, 2700, 2800, 2900, 3000, 3100, 3200, 3300, 3400, 3500, 3600, 3700, 3800, 3900, or 4000 (h·ng/mL). In one embodiment, the mean AUC_{inf}(h·ng/mL) measured over 72.5 hours after administration is about 3160 h·ng/mL±522 h·ng/mL. In one embodiment, the mean AUC_{inf} (h·ng/mL) measured over 72.5 hours after administration is about 2500 h·ng/mL, is about 2600 h·ng/mL, about 2900 h·ng/mL, about 3000 h·ng/mL, about 3050 h·ng/mL, about 3100 h·ng/mL, about 3250 h·ng/mL, about 3300 h·ng/mL, about 3500 h·ng/mL, about 3600 h·ng/mL, about 3700 h·ng/mL, or about 3800 h·ng/mL. In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a AUC_{inf} (h·ng/mL) as described herein.

In another embodiment, the compounds described herein are orally or intravenously administered to a subject prior to administration of a chemotherapeutic agent so that a single dose of the compounds described herein provides a blood plasma level profile with a mean AUC_{inf}(h·ng/mL) measured over 24.5 hours after administration of between about 2379 h·ng/mL to about 3762 h·ng/mL or about 1530 h·ng/mL to about 3300 h·ng/mL. In one embodiment, the mean AUC_{inf} h·ng/mL measured over 24.5 hours after administration is about at least 2991 h·ng/mL or about at least 2140 h·ng/mL. In one embodiment, the mean AUC_{inf} h·ng/mL measured over 72.5 hours after administration of between about 2379 h·ng/mL to about 3762 h·ng/mL or about 1530 h·ng/mL to about 3300 h·ng/mL. In one embodiment, the mean AUC_{inf} h·ng/mL measured over 72.5 hours after administration is about at least 2991 h·ng/mL or about at least 2140 h·ng/mL.

In another embodiment, the compounds described herein are orally or intravenously administered to a subject prior to administration of a chemotherapeutic agent so that a single dose of the compounds described herein provides a blood plasma level profile with a mean AUCₜ (ng·hr/mL) measured over 24.5 hours after administration of between about 2000 h·ng/mL to about 4500 h·ng/mL, between about 2600 h·ng/mL to about 3700 h·ng/mL, between about 2800 h·ng/mL to about 3500 h·ng/mL, or between about 3000 h·ng/mL to about 3200 h·ng/mL. In one embodiment, the mean AUCₜ(ng·hr/mL) measured over 24.5 hours after administration is about 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, or 4500 (h·ng/mL). In one embodiment, the mean AUCₜ(ng·hr/mL) measured over 24.5 hours after administration is about 2830 (ng·hr/mL)±474 (ng·hr/mL). In one embodiment, the mean AUCₜ(ng·hr/mL) measured over 72.5 hours after administration is between about 2000 h·ng/mL to about 4500 h·ng/mL, between about 2600 h·ng/mL to about 3700 h·ng/mL, between about 2800 h·ng/mL to about 3500 h·ng/mL, or between about 3000 h·ng/mL to about 3200 h·ng/mL. In one embodiment, the mean AUCₜ(ng·hr/mL) measured over about 72.5 hours after administration is about 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, or 4500 (ng·hr/mL). In one embodiment, the mean AUCₜ(ng·hr/mL) measured over 72.5 hours after administration is about 3110 (ng·hr/mL)±515 (ng·hr/mL). In one embodiment, the mean AUCₜ(ng·hr/mL) measured over 72.5 hours after administration is about 3000 (ng·hr/mL), is about 3050 (ng·hr/mL), is about 3100 (ng·hr/mL), is about 3110 (ng·hr/mL), is about 3150 (ng·hr/mL), is about 3200 (ng·hr/mL), or is about 3250 (ng·hr/mL). In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m².

In another embodiment, the compounds described herein are orally or intravenously administered to a subject prior to administration of a chemotherapeutic agent so that a single dose of the compounds described herein provides a blood plasma level profile with a mean AUCₜ (ng·hr/mL) of between about 2360 h·ng/mL to about 3750 h·ng/mL or about 1530 h·ng/mL to about 3300 h·ng/mL. In one embodiment, the mean AUCₜ(ng·hr/mL) is about at least 2991 h·ng/mL or about at least 2140 h·ng/mL.

In another embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a mean AUCₜ(ng·hr/mL) measured over about 24.5 hours after administration of between about 2300 h·ng/mL to about 4000 h·ng/mL. In one embodiment, the mean AUCₜ (ng·hr/mL) measured over about 24.5 hours after administration is about 2300, 2400, 2500, 2600, 2700, 2800, 2900, 3000, 3100, 3200, 3300, 3400, 3500, 3600, 3700, 3800, 3900, or 4000 (ng·hr/mL). In one embodiment, the mean AUCₜ(ng·hr/mL) measured over about 24.5 hours after administration is about 2830 (ng·hr/mL)±550 (ng·hr/mL). In an alternative embodiment, the mean AUCₜ(ng·hr/mL) measured over about 24.5 hours after administration is about 2830 (ng·hr/mL)±560 (ng·hr/mL) or about 2830 (ng·hr/mL)±about 20%. In one embodiment, the mean AUCₜ(ng·hr/mL) measured over about 24.5 hours after administration is about 3020 (ng·hr/mL) about 20%. In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a mean AUCₜ(ng·hr/mL) measured over about 24.5 hours after administration of at least about 2040 ng·hr/mL. In one embodiment, the mean AUCₜ(ng·hr/mL) measured over about 72.5 hours after administration is between about 2300 h·ng/mL to about 4100 h·ng/mL. In one embodiment, the mean AUCₜ(ng·hr/mL) measured over about 72.5 hours after administration is about 2300, 2400, 2500, 2600, 2700, 2800, 2900, 3000, 3100, 3200, 3300, 3400, 3500, 3600, 3700, 3800, 3900, 4000, or 4100 (ng·hr/mL). In an alternative embodiment, the mean AUCₜ(ng·hr/mL) measured over about 72.5 hours after administration is about 3100 (ng·hr/mL) 620 (ng·hr/mL) or about 3100 (ng·hr/mL)±about 20%. In one embodiment, the mean AUCₜ(ng·hr/mL) measured over about 72.5 hours after administration is about 3410 (ng·hr/mL)±about 20%. In one embodiment, the compounds described herein are administered on days 1 and 2 of the treatment regime. In one embodiment, the compounds described herein are administered on days 1, 2, and 3 of the treatment regime. In one embodiment, the compounds described herein are administered on days 1, 2, 3, and 4 of the treatment regime. In one embodiment, the compounds described herein are administered on days 1, 2, 3, 4, and 5 of the treatment regime.

In another embodiment, the compounds described herein are orally or intravenously administered to a subject prior to administration of a chemotherapeutic agent so that a single dose of the compounds described herein provides a blood plasma level profile with a dosage-corrected mean AUCₜ(h·ng/mL)/(mg/m²) of between about 6 (h·ng/mL)/(mg/m²) and 20 (h·ng/mL)/(mg/m²), of between about 8 (h·ng/mL)/(mg/m²) and 15 (h·ng/mL)/(mg/m²), of between about 10 (h·ng/mL)/(mg/m²) and 13 (h·ng/mL)/(mg/m²). In one embodiment, the dosage-corrected mean AUCₜ(h·ng/mL)/(mg/m²) is about 6, 7, 8, 9, 10, 11, 12, 13, 14, 15, 16, 17, 18, 19, or 20 (h·ng/mL)/(mg/m²). In one embodiment, the dosage-corrected mean AUCₜ (h·ng/mL)/(mg/m²) is about 12.5 (h·ng/mL)/(mg/m²)±2.2 (h·ng/mL)/(mg/m²). In one embodiment, the dosage-corrected mean AUCₜ(h·ng/mL)/(mg/m²) is about 10.5 (h·ng/mL)/(mg/m²), about 11.0 (h·ng/mL)/(mg/m²), about 11.5 (h·ng/mL)/(mg/m²), about 12.0 (h·ng/mL)/(mg/m²), about 12.5 (h·ng/mL)/(mg/m²), or about 13.0 (h·ng/mL)/(mg/m²). The dosage corrected mean AUCₜ is mean AUCₜ divided by the number of milligrams/m² of the compounds described herein in the formulation. In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m².

In another embodiment, the compounds described herein are orally or intravenously administered to a subject prior to administration of a chemotherapeutic agent so that a single dose of the compounds described herein provides a blood plasma level profile with a dosage-corrected mean AUCₜ(h·ng/mL)/(mg/m²) of between about 12.3 (h·ng/mL)/(mg/m²) to about 19.5 (h·ng/mL)/(mg/m²) or about 7.6 (h·ng/mL)/(mg/m²) to about 16.5 (h·ng/mL)/(mg/m²). In one embodiment, the dosage-corrected mean AUCₜ(h·ng/mL)/(mg/m²) is about at least 15.6 (h·ng/mL)/(mg/m²) or about at least 10.7 (h·ng/mL)/(mg/m²).

In another embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a dosage-corrected mean AUCₜ(h·ng/mL)/(mg/m²) of between about 6 (h·ng/mL)/(mg/m²) and 20 (h·ng/mL)/(mg/m²). In one embodiment, the dosage-corrected mean AUCₜ(h·ng/mL)/(mg/m²) is about 15.0 (h·ng/mL)/(mg/m²)±3.0 (h·ng/mL)/(mg/m²) or about 15.0 (h·ng/mL)/(mg/m²)±about 20%. In one embodiment, the dosage-corrected mean AUCₜ (h·ng/mL)/(mg/m²) is at least about 8.35 (h·ng/mL)/(mg/m²). In one embodiment, the dosage-corrected mean AUCₜ(h·ng/mL)/(mg/m²) is about 16.5 (h·ng/mL)/(mg/m²)±about 20%. In one embodiment, the dosage-corrected mean AUCₜ(h·ng/mL)/(mg/m²) is at least about 10.0 (h·ng/mL)/(m g/m²). The dosage corrected AUCₜ is AUCₜ divided by the number of milligrams/m² of the compounds described herein in the formulation. In one embodiment, the compounds described herein are administered on days 1 and 2 of the treatment regime. In one embodiment, the compounds described herein are administered on days 1, 2, and 3 of the treatment regime. In one embodiment, the compounds described herein are administered on days 1, 2, 3, and 4 of the treatment regime. In one embodiment, the compounds described herein are administered on days 1, 2, 3, 4, and 5 of the treatment regime.

In another embodiment, the compounds described herein are orally or intravenously administered to a subject prior to administration of a chemotherapeutic agent so that a single dose of the compounds described herein provides a blood plasma level profile with a dosage-corrected mean AUC_{inf} (h·ng/mL)/(mg/m²) of between about 6 (h·ng/mL)/(mg/m²) and 20 (h·ng/mL)/(mg/m²), of between about 8 (h·ng/mL)/(mg/m²) and 15 (h·ng/mL)/(mg/m²), of between about 10 (h·ng/mL)/(mg/m²) and 13 (h·ng/mL)/(mg/m²). In one embodiment, the dosage-corrected mean AUC_{inf} (h·ng/mL)/(mg/m²) is about 6, 7, 8, 9, 10, 11, 12, 13, 14, 15, 16, 17, 18, 19, or 20 (h·ng/mL)/(mg/m²). In one embodiment, the dosage-corrected mean AUC_{inf}(h·ng/mL)/(mg/m²) is about 12.7 (h·ng/mL)/(mg/m²)±2.5 (h·ng/mL)/(mg/m²). In one embodiment, the dosage-corrected mean AUC_{inf} (h·ng/mL)/(mg/m²) is about 10.5 (h·ng/mL)/(mg/m²), about 11.0 (h·ng/mL)/(mg/m²), about 11.5 (h·ng/mL)/(mg/m²), about 12.0 (h·ng/mL)/(mg/m²), about 12.5 (h·ng/mL)/(mg/m²), about 13.0 (h·ng/mL)/(mg/m²), or about 13.5 (h·ng/mL)/(mg/m²). The dosage corrected mean AUC_{inf} is mean AUC_{inf} divided by the number of milligrams/m² of the compounds described herein in the formulation. In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m².

In another embodiment, the compounds described herein are orally or intravenously administered to a subject prior to administration of a chemotherapeutic agent so that a single dose of the compounds described herein provides a blood plasma level profile with a dosage-corrected mean AUC_{inf} (h·ng/mL)/(mg/m²) measured over 24.5 hours after administration of between about 12.4 (h·ng/mL)/(mg/m²) to about 19.6 (h·ng/mL)/(mg/m²) or about 7.6 (h·ng/mL)/(mg/m²) to about 16.5 (h·ng/mL)/(mg/m²). In one embodiment, the mean AUC_{inf}(h·ng/mL)/(mg/m²) measured over 24.5 hours after administration is about at least 15. (h·ng/mL)/(mg/m²) or about at least 10.7 (h·ng/mL)/(mg/m²). In one embodiment, the mean AUC_{inf}(h·ng/mL)/(mg/m²) measured over 72.5 hours after administration of between about 12.4 (h·ng/mL)/(mg/m²) to about 19.6 (h·ng/mL)/(mg/m²) or about 7.6 (h·ng/mL)/(mg/m²) to about 16.5 (h·ng/mL)/(mg/m²). In one embodiment, the mean AUC_{inf} (h·ng/mL)/(mg/m²) measured over 72.5 hours after administration is about at least 15.6 h-(h·ng/mL)/(mg/m²) or about at least 10.7 (h·ng/mL)/(mg/m²).

In another embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a dosage-corrected mean AUCₜ(h·ng/mL)/(mg/m²) of between about 6 (h·ng/mL)/(mg/m²) and 20 (h·ng/mL)/(mg/m²). In one embodiment, the dosage-corrected mean AUCₜ (h·ng/mL)/(mg/m²) is 6, 7, 8, 9, 10, 11, 12, 13, 14, 15, 16, 17, 18, 19, or 20 (h·ng/mL)(mg/m²). In one embodiment, the dosage-corrected mean AUCₜ(h·ng/mL)/(mg/m²) is about 15.0 (h·ng/mL)/(mg/m²)±3.0 (h·ng/mL)/(mg/m²) or about 15.0 (h·ng/mL)/(mg/m²)±about 20%. In one embodiment, the dosage-corrected mean AUCₜ(h·ng/mL)/(mg/m²) is about 8.35 (h·ng/mL)/(mg/m²)±about 20%. In one embodiment, the dosage-corrected mean AUCₜ (h·ng/mL)/(mg/m²) is about 16.5 (h·ng/mL)/(mg/m²)±about 20%. In one embodiment, the dosage-corrected mean AUCₜ(h·ng/mL)/(mg/m²) is at least about 10.0 (h·ng/mL)/(mg/m²). The dosage corrected AUCₜ is AUCₜ divided by the number of milligrams/m² of the compounds described herein in the formulation. In one embodiment, the compounds described herein are administered on days 1 and 2 of the treatment regime. In one embodiment, the compounds described herein are administered on days 1, 2, and 3 of the treatment regime. In one embodiment, the compounds described herein are administered on days 1, 2, 3, and 4 of the treatment regime. In one embodiment, the compounds described herein are administered on days 1, 2, 3, 4, and 5 of the treatment regime.

In another embodiment, the compounds described herein are orally or intravenously administered to a subject prior to administration of a chemotherapeutic agent so that a single dose of the compounds described herein provides a blood plasma level profile with a with a mean CL (L/h/m²) measured over 24.5 hours after administration of between about 45 L/h/m² and about 85 L/h/m². In one embodiment, the mean CL (L/h/m²) measured over 24.5 hours after administration is 45, 50, 55, 60, 65, 70, 75, 80, or 85 (L/h/m²). In one embodiment, the mean CL (L/h/m²) measured over 24.5 hours after administration is about 65 (L/h/m²)±15 (L/h/m²). In one embodiment, the mean CL (L/h/m²) measured over 24.5 hours after administration is about 64.4 (L/h/m²)±10.6 (L/h/m²). In one embodiment, the mean CL (L/h/m²) measured over 72.5 hours after administration is between about 45 (L/h/m²) to about 80 (L/h/m²). In one embodiment, the mean CL (L/h/m²) measured over 72.5 hours after administration is about 60 (L/h/m²)±15 (L/h/m²). In one embodiment, the mean CL (L/h/m²) measured over 72.5 hours after administration is about 62.1 (L/h/m²)±10.3 (L/h/m²). In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a CL (L/h/m²) as described herein.

In another embodiment, provided is a method of treating a subject undergoing chemotherapy for the treatment of a CDK 4/6-replication independent cellular proliferation disorder by providing an intravenous administered formulation of the compounds described herein wherein a single-dose provides a blood plasma level profile with a mean Vₛₛ (L/m²) measured over 24.5 hours after final administration of between about 320 (L/m²) and about 630 (L/m²). In one embodiment, the mean Vₛₛ (L/m²) measured over 24.5 hours is 320, 370, 400, 420, 470, 500, 520, 570, 600, or 630 (L/m²). In one embodiment, the mean Vₛₛ (L/m²) measured over 24.5 hours is about 425 (L/m²)±150 (L/m²). In one embodiment, the mean Vₛₛ (L/m²) measured over 24.5 hours in about 421 (L/m²)±101 (L/m²). In one embodiment, the mean Vₛₛ (L/m²) measured over 72.5 hours after final administration of between about 390 (L/m²) and about 825 (L/m²). In one embodiment, the mean Vₛₛ (L/m²) measured over 72.5 hours after final administration is 400, 450, 500, 550, 600, 650, 700, 750, 800, or 825 (L/m²). In one embodiment, the mean Vₛₛ (L/m²) measured over 72.5 hours after final administration is about 550 (L/m²)±175 (L/m²). In one embodiment, the mean Vₛₛ (L/m²) measured over 72.5 hours after final administration is about 547 (L/m²)±147 (L/m²). In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a Vₛₛ (L/m²) as described herein.

In another embodiment, the compounds described herein are orally or intravenously administered to a subject prior to administration of a chemotherapeutic agent so that a single dose of the compounds described herein provides a blood plasma level profile with a mean MRTs (h) measured over 24.5 hours of between about 4.75 (h) and about 9.25 (h). In one embodiment, the mean MRT_{inf} (h) measured over 24.5 hours is 4.75, 5.25, 5.75, 6.25, 6.75, 7.25, 7.75, 8.25, 8.75, or 9.25 (h). In one embodiment, the mean MRT_{inf} (h) measured over 24.5 hours is about 6.5 (h)±1.75 (h). In one embodiment, the mean MRT_{inf} (h) measured over 24.5 hours is about 6.59 (h)±1.33 (h). In one embodiment, the mean MRT_{inf} (h) measured over 72.5 hours is between about 6 (h) and about 13 (h). In one embodiment, the mean MRT_{inf} (h) measured over 72.5 hours is about 9 (h)±2.5 (h). In one embodiment, the mean MRT_{inf} (h) measured over 72.5 hours is about 8.86 (h)±2.12 (h). In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a Vₛₛ (L/m²) as described herein.

In another embodiment, the compounds described herein are orally or intravenously administered to a subject prior to administration of a chemotherapeutic agent so that a single dose of the compounds described herein provides a blood plasma level profile with a mean λz (1/h) measured over 24.5 hours of between about 0.07 and 0.15. In one embodiment, the mean λz (1/h) measured over 24.5 hours is 0.07, 0.08, 0.09, 0.10, 0.11, 0.12, 0.13, 0.14, or 0.15 (1/h). In one embodiment, the mean λz (1/h) measured over 24.5 hours is about 0.09±0.025. In one embodiment, the λz mean (1/h) measured over 24.5 hours is about 0.0899±0.0157. In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a λz (1/h) measured over 24.5 hours as described herein.

In another embodiment, the compounds described herein are orally or intravenously administered to a subject prior to administration of a chemotherapeutic agent so that a single dose of the compounds described herein provides a blood plasma level profile with a mean t½ (h) measured over 24.5 hours of between about 5 h and 9.5 h. In one embodiment, the mean t½ (h) measured over 24.5 hours is 5, 5.5, 6, 6.5, 7, 7.5, 8, 8.5, 9, or 9.5 h. In one embodiment, the mean t½ (h) measured over 24.5 hours is about 8±1.5 (h). In one embodiment, the mean t½ (h) measured over 24.5 hours is about 7.87±1.14 (h). In one embodiment, the mean t½β (h) measured over 72.5 hours is between about 5.5 (h) and about 9 (h). In one embodiment, the mean t½β (h) measured over 24.5 hours is 5, 5.5, 6, 6.5, 7, 7.5, 8, 8.5, or 9 h. In one embodiment, the mean t½β (h) measured over 72.5 hours is about 8 (h)±1.5 (h). In one embodiment, the mean t½β (h) measured over 72.5 hours is about 7.87 (h)±1.14 (h). In one embodiment, the mean t½γ (h) measured over 72.5 hours is between about 15 (h) and about 22 (h). In one embodiment, the mean t½γ (h) measured over 72.5 hours is 15, 16, 17, 18, 19, 20, 21, or 22 (h). In one embodiment, the mean t½γ (h) measured over 72.5 hours is about 18 (h)±2.25 (h). In one embodiment, the mean t½γ (h) measured over 72.5 hours is about 18.0 (h)±1.92 (h). In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a t½ (h), t1/2β (h), and/or t½γ (h) measured over 24.5 hours and/or 72.5 hours as described herein.

In another embodiment, the compounds described herein are orally or intravenously administered to a subject prior to administration of a chemotherapeutic agent so that a single dose of the compounds described herein provides a blood plasma level profile with a mean t½ (h) of between about 11.9 h and 17.3 h.

In one embodiment, the compounds described herein are orally or intravenously administered to a subject prior to administration of a chemotherapeutic agent so that a single dose of the compounds described herein provides a blood plasma level profile with a mean concentration (ng/mL) at 24.5 hours after the end of administration of between about 5 (ng/mL) and about 35 (ng/mL). In one embodiment, the mean concentration at 24.5 hours after the end of administration is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 (ng/mL). In one embodiment, the mean concentration at 24.5 hours after the end of administration is about 19 (ng/mL)±5.24 (ng/mL). In one embodiment, the mean concentration at 24.5 hours after the end of administration is about 20 (ng/mL)±7.5 (ng/mL). In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a with a mean concentration (ng/mL) at 24.5 hours after the end of administration as described herein.

In another embodiment, the compounds described herein are orally or intravenously administered to a subject prior to administration of a chemotherapeutic agent so that a single dose of the compounds described herein provides a blood plasma level profile with a mean concentration (ng/mL) at 72.5 hours after the end of administration of between about 0.7 (ng/mL) and about 3 (ng/mL). In one embodiment, the mean concentration at 72.5 hours after the end of administration is about 0.7, 0.9, 1.0, 1.2, 1.4, 1.6, 1.8, 2.0, 2.2, 2.4, 2.6, 2.8, or 3 (ng/mL). In one embodiment, the mean concentration at 72.5 hours after the end of administration is about 2.25 (ng/mL)±1.5 (ng/mL). In one embodiment, the mean concentration at 72.5 hours after the end of administration is about 1.79 (ng/mL)±0.731 (ng/mL). In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a with a mean concentration (ng/mL) at 72.5 hours after the end of administration as described herein.

In another embodiment, the compounds described herein are orally or intravenously administered to a subject prior to administration of a chemotherapeutic agent so that a single dose of the compounds described herein provides a primary pharmacokinetic blood plasma level profile with a mean α (1/h) of between about 1 (1/h) and 15 (1/h). In one embodiment, a single-dose provides a primary pharmacokinetic blood plasma level profile with a mean α (1/h) of 1, 2, 3, 4, 5, 6, 7, 8, 9, 10, 11, 12, 13, 14, 15 (1/h). In one embodiment, a single-dose provides a primary pharmacokinetic blood plasma level profile with a mean α (1/h) of about 11 (1/h)±9 (1/h). In one embodiment, a single-dose provides a primary pharmacokinetic blood plasma level profile with a mean α (1/h) of about 11.3 (1/h)±7.06 (1/h). In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a with a mean α (1/h) as described herein.

In another embodiment, the compounds described herein are orally or intravenously administered to a subject prior to administration of a chemotherapeutic agent so that a single dose of the compounds described herein provides a primary pharmacokinetic blood plasma level profile with a mean β (1/h) of about 0.4 (1/h)±0.3 (1/h). In one embodiment, provided is a primary pharmacokinetic blood plasma level profile with a mean β (1/h) of about 0.362 (1/h)±0.110 (1/h). In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a with a mean β (1/h) as described herein.

In another embodiment, the compounds described herein are orally or intravenously administered to a subject prior to administration of a chemotherapeutic agent so that a single dose of the compounds described herein provides a primary pharmacokinetic blood plasma level profile with a mean γ (1/h) of about 0.05 (1/h)±0.01 (1/h). In one embodiment, provided is a primary pharmacokinetic blood plasma level profile with a mean γ (1/h) of about 0.0497 (1/h)±0.00442 (1/h). In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a with a mean γ (1/h) as described herein.

In another embodiment, the compounds described herein are orally or intravenously administered to a subject prior to administration of a chemotherapeutic agent so that a single dose of the compounds described herein provides a 3-compartment primary pharmacokinetic blood plasma level profile with a mean K21 (1/h) of about 1 (1/h)±0.6. In one embodiment, a single-dose provides a 3-compartment primary pharmacokinetic blood plasma level profile with a mean K21 (1/h) of about 0.993 (1/h)±0.439. In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a with a mean K21 (1/h) as described herein.

In another embodiment, the compounds described herein are orally or intravenously administered to a subject prior to administration of a chemotherapeutic agent so that a single dose of the compounds described herein provides a 3-compartment primary pharmacokinetic blood plasma level profile with a mean K31 (1/h) of about 0.08 (1/h)±0.03 (1/h). In one embodiment, a single-dose provides a 3-compartment primary pharmacokinetic blood plasma level profile with a mean K31 (1/h) of about 0.0750 (1/h)±0.0160 (1/h). In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a with a mean K31 (1/h) as described herein.

In another embodiment, the compounds described herein are orally or intravenously administered to a subject prior to administration of a chemotherapeutic agent so that a single dose of the compounds described herein provides a 3-compartment primary pharmacokinetic blood plasma level profile with a mean V1 (L/m²) of about 25±15. In one embodiment, a single-dose provides a 3-compartment primary pharmacokinetic blood plasma level profile with a mean V1 (L/m²) of about 25.6±9.51. In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a with a mean V1 (L/m²) as described herein.

In another embodiment, the compounds described herein are orally or intravenously administered to a subject prior to administration of a chemotherapeutic agent so that a single dose of the compounds described herein provides a 3-compartment primary pharmacokinetic blood plasma level profile with a mean Cₘₐₓ (ng/mL) of about 2000 (ng/mL)±650 (ng/mL). In one embodiment, a single-dose provides a 3-compartment primary pharmacokinetic blood plasma level profile with a mean Cₘₐₓ (ng/mL) of about 2020 (ng/mL)±505 (ng/mL). In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a with a mean Cₘₐₓ(ng/mL) as described herein.

In another embodiment, the compounds described herein are orally or intravenously administered to a subject prior to administration of a chemotherapeutic agent so that a single dose of the compounds described herein provides a primary pharmacokinetic blood plasma level profile with a mean t½α of about 0.1 (h)±0.05 (h). In one embodiment, a single-dose provides a primary pharmacokinetic blood plasma level profile with a mean t % a of about 0.0776 (h)±0.0329 (h). In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a with a mean t½α (h) as described herein.

In another embodiment, the compounds described herein are orally or intravenously administered to a subject prior to administration of a chemotherapeutic agent so that a single dose of the compounds described herein provides a 2-compartment primary pharmacokinetic blood plasma level profile with a mean t½β of about 2 (h)±0.75 (h). In one embodiment, a single-dose provides a 2-compartment primary pharmacokinetic blood plasma level profile with a mean t½β of about 2.03 (h)±0.444 (h). In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a with a mean t1/2β (h) as described herein.

In another embodiment, the compounds described herein are orally or intravenously administered to a subject prior to administration of a chemotherapeutic agent so that a single dose of the compounds described herein provides a 3-compartment primary pharmacokinetic blood plasma level profile with a mean t½γ of about 15 (h)±3 (h). In one embodiment, a single-dose provides a 3-compartment primary pharmacokinetic blood plasma level profile with a mean t½γ of about 14.0 (h)±1.35 (h). In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a with a mean t½γ (h) as described herein.

In another embodiment, the compounds described herein are orally or intravenously administered to a subject prior to administration of a chemotherapeutic agent so that a single dose of the compounds described herein provides a 3-compartment primary pharmacokinetic blood plasma level profile with a mean AUC (h·ng/mL) of about 3200 (h·ng/mL)±750 (h·ng/mL).

In one embodiment, a single-dose provides a 3-compartment primary pharmacokinetic blood plasma level profile with a mean AUC (h·ng/mL) of about 3220 (h·ng/mL)±559 (h·ng/mL). In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a with a mean AUC (h·ng/mL) as described herein.

In another embodiment, the compounds described herein are orally or intravenously administered to a subject prior to administration of a chemotherapeutic agent so that a single dose of the compounds described herein provides a 3-compartment primary pharmacokinetic blood plasma level profile with a mean CL (L/h/m²) of about 60 (L/h/m²)±15 (L/h/m²). In one embodiment, a 3-compartment primary pharmacokinetic blood plasma level profile with a mean CL (L/h/m²) of about 61.1 (L/h/m²)±10.6 (L/h/m²). In one embodiment, the single dose is between about 170 mg/m² and 240 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a with a mean CL (L/h/m²) as described herein.

In another embodiment, the compounds described herein are orally or intravenously administered to a subject prior to administration of a chemotherapeutic agent so that a single dose of the compounds described herein provides a 3-compartment primary pharmacokinetic blood plasma level profile with a mean Vₛₛ (L/m²) of about 500 (L/m²)±200 (L/m²). In one embodiment, a 3-compartment primary pharmacokinetic blood plasma level profile with a mean Vₛₛ (L/m²) of about 508 (L/m²)±131 (L/m²). In one embodiment, the single dose is between about 170 mg/m² and 280 mg/m² or 170 mg/m² and 215 mg/m². In one embodiment, the single dose is about 190 mg/m². In one embodiment, the single dose is about 200 mg/m². In one embodiment, the single dose is about 240 mg/m². In one embodiment, the compounds described herein are administered to a subject prior to administration of a chemotherapeutic agent in a multi-day chemotherapeutic treatment regime, for example, 2 days, 3 days, 4 days, or 5 days, wherein the compounds described herein, following administration on any day of the multi-day chemotherapeutic treatment regime, for example day 2, 3, 4, or 5, provides a blood plasma level profile of the compounds described herein with a with a mean Vₛₛ (L/m²) as described herein.

In another embodiment, the compounds described herein at the dosages described about is administered daily for more than 1, more than 2, more than 3, more than 4, more than 5, more than 6, more than 7, more than 8, more than 9, more than 10, more than 11, more than 12, more than 13, more than 14, more than 15, more than 16, more than 17, more than 18, more than 19, more than 20, more than 21, more than 22, more than 23, more than 24, more than 25 more than 26, more than 27, or more than 28 days. In one embodiment, provided is a method of treating a subject undergoing chemotherapy for the treatment of a CDK 4/6-replication independent cellular proliferation disorder by providing an intravenous administered formulation of the compounds described herein at a dosage described herein daily for 1, 2, 3, 4, 5, 6, 7, 8, 9, 10, 11, 12, 13, 14, 15, 16, 17, 18, 19, 20, 21, 22, 23, 24, 25, 26, 27, or 28 days or more.

In another embodiment, provided is a method of treating a subject undergoing chemotherapy for the treatment of a CDK 4/6-replication independent cellular proliferation disorder by providing an intravenous administered formulation of the compounds described herein wherein a single-dose of the compounds described herein followed by a single-dose of Topotecan at 1.5 mg/m² provides a mean Cₘₐₓ (ng/mL) of Topotecan between about 30 ng/mL to about 150 ng/mL. In one embodiment, the mean Cₘₐₓ (ng/mL) of Topotecan at 1.5 mg/m² is about 30 ng/mL, about 40 ng/mL, about 50 ng/mL, about 60 ng/mL, about 70 ng/mL, about 80 ng/mL, about 90 ng/mL, about 100 ng/mL. about 110 ng/mL, about 120 ng/mL, about 130 ng/mL, about 140 ng/mL or about 150 ng/mL. In one embodiment, a single-dose of the compounds described herein followed by a single-dose of Topotecan at 1.25 mg/m² provides a mean Cₘₐₓ (ng/mL) of Topotecan between about 20 ng/mL and 120 ng/mL. In one embodiment, the mean Cₘₐₓ (ng/mL) of Topotecan at 1.25 mg/m² is about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 110 ng/mL, or about 120 ng/mL. In one embodiment, a single-dose of the compounds described herein followed by a single-dose of Topotecan at 0.75 mg/m² provides a mean Cₘₐₓ (ng/mL) of Topotecan between about 10 ng/mL and 70 ng/mL. In one embodiment, the mean Cₘₐₓ (ng/mL) of Topotecan at 0.75 mg/m² is about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, or about 70 ng/mL.

In another embodiment, provided is a method of treating a subject undergoing chemotherapy for the treatment of a CDK 4/6-replication independent cellular proliferation disorder by providing an intravenous administered formulation of the compounds described herein wherein a single-dose of the compounds described herein followed by a single-dose of Topotecan at 1.5 mg/m² provides a mean Cₘₐₓ (ng/mL) of Topotecan between about 40.2 ng/mL and about 122 ng/mL. In one embodiment, a single-dose of the compounds described herein followed by a single-dose of Topotecan at 1.25 mg/m² provides a mean Cₘₐₓ (ng/mL) of Topotecan between about 33.1 ng/mL and 104 ng/mL. In one embodiment, a single-dose of the compounds described herein followed by a single-dose of Topotecan at 0.75 mg/m² provides a mean Cₘₐₓ (ng/mL) of Topotecan between about 17.9 ng/mL and 38.5 ng/mL.

In another embodiment, provided is a method of treating a subject undergoing chemotherapy for the treatment of a CDK 4/6-replication independent cellular proliferation disorder by providing an intravenous administered formulation of the compounds described herein wherein a single-dose of the compounds described herein followed by a single-dose of Topotecan at 1.5 mg/m² provides a mean AUC, (h·ng/mL) of Topotecan between about 100 h·ng/mL and about 300 h·ng/mL. In one embodiment, the mean AUC, (h·ng/mL) of Topotecan at 1.5 mg/m² is about 100 h·ng/mL, about 110 h·ng/mL, about 120 h·ng/mL, about 130 h·ng/mL, about 140 h·ng/mL, about 150 h·ng/mL, about 160 h·ng/mL, about 170 h·ng/mL, about 180 h·ng/mL, about 190 h·ng/mL, about 200 h·ng/mL, about 220 h·ng/mL, about 240 h·ng/mL, about 260 h·ng/mL, about 280 h·ng/mL, or about 300 h·ng/mL. In one embodiment, a single-dose of the compounds described herein followed by a single-dose of Topotecan at 1.25 mg/m² provides a mean AUC, (h·ng/mL) of Topotecan between about 80 h·ng/mL and about 300 h·ng/mL. In one embodiment, the mean AUC, (h·ng/mL) of Topotecan at 1.25 mg/m² is about 80 h·ng/mL, about 90 h·ng/mL, about 100 h·ng/mL, about 110 h·ng/mL, about 120 h·ng/mL, about 130 h·ng/mL, about 140 h·ng/mL, about 150 h·ng/mL, about 160 h·ng/mL, about 170 h·ng/mL, about 180 h·ng/mL, about 190 h·ng/mL, about 200 h·ng/mL, about 220 h·ng/mL, about 240 h·ng/mL, about 260 h·ng/mL, about 280 h·ng/mL, or about 300 h·ng/mL. In one embodiment, a single-dose of the compounds described herein followed by a single-dose of Topotecan at 0.75 mg/m² provides a mean AUC, (h·ng/mL) of Topotecan between about 50 h·ng/mL and about 200 h·ng/mL. In one embodiment, the mean AUC, (h·ng/mL) of Topotecan at 0.75 mg/m² is about 50 h·ng/mL, about 60 h·ng/mL, 70 h·ng/mL, about 80 h·ng/mL, about 90 h·ng/mL, about 100 h·ng/mL, about 110 h·ng/mL, about 120 h·ng/mL, about 130 h·ng/mL, about 140 h·ng/mL, about 150 h·ng/mL, about 160 h·ng/mL, about 170 h·ng/mL, about 180 h·ng/mL, about 190 h·ng/mL, or about 200 h·ng/mL.

In another embodiment, provided is a method of treating a subject undergoing chemotherapy for the treatment of a CDK 4/6-replication independent cellular proliferation disorder by providing an intravenous administered formulation of the compounds described herein wherein a single-dose of the compounds described herein followed by a single-dose of Topotecan at 1.5 mg/m² provides a mean AUC_{τ} (h·ng/mL) of Topotecan between about 132 h·ng/mL and about 181 h·ng/mL. In one embodiment, a single-dose of the compounds described herein followed by a single-dose of Topotecan at 1.25 mg/m² provides a mean AUC_{τ} (h·ng/mL) of Topotecan between about 121 h·ng/mL and about 254 h·ng/mL. In one embodiment, a single-dose of the compounds described herein followed by a single-dose of Topotecan at 0.75 mg/m² provides a mean AUC_{τ} (h·ng/mL) of Topotecan between about 74.4 h·ng/mL and about 120 h·ng/mL.

As provided herein, the compounds described herein can be administered wherein any one or more of the described PK or PD blood profile parameters described herein is reached to treat a subject undergoing chemotherapy for the treatment of any CDK-replication independent cellular proliferation disorder.

In another embodiment, provided is a method of treating a subject having a CDK-replication dependent cellular proliferation disorder by providing an intravenous administered formulation of the compounds described herein in a dosage providing a blood plasma profile as described herein.

In another embodiment, provided is method of treating a subject having a CDK-replication independent cellular proliferation disorder by providing an intravenous administered formulation of the compounds described herein in a dosage providing a combination of two or more blood plasma parameters at levels described herein. Non-limiting examples of parameters that can be provided in combinations of two or more at levels described herein include: mean Cₘₐₓ, mean dosage-corrected Cₘₐₓ, mean Tₘₐₓ, mean AUC_{inf}, dosage-corrected mean AUC_{inf}, mean AUCₜ, dosage-corrected mean AUCₜ, and mean t½.

Another embodiment described herein is a kit for dispensing a pharmaceutical dosage form comprising any of the compounds described herein, the kit comprising: (a) at least one dosage form comprising a compound described herein; (b) at least one moisture proof dispensing receptacle comprising blister or strip packs, an aluminum blister, a transparent or opaque polymer blister with pouch, polypropylene tubes, colored blister materials, tubes, bottles, and bottles optionally containing a child-resistant feature, optionally comprising a desiccant, such as a molecular sieve or silica gel; and optionally (c) an insert comprising instructions or prescribing information for the compound 1 comprised by the oral pharmaceutical composition; or (d) directions for administration or any contraindications.

Another embodiment is a kit comprising one or more pre-filled syringes comprising a solution or suspension of one or more compounds described herein. In one embodiment, such a kit comprises a pre-filled syringe comprising compounds described herein in a blister pack or a sealed sleeve. The blister pack or sleeve may be sterile on the inside. In one aspect, pre-filled syringes as described herein may be placed inside such blister packs or sleeves prior to undergoing sterilization, for example terminal sterilization.

Such a kit may further comprise one or more needles for administration of the compounds described herein. Such kits may further comprise instructions for use, a drug label, contraindications, warnings, or other relevant information. One embodiment described herein is a carton or package comprising one or more pre-filled syringes comprising one or more compounds as described herein contained within a blister pack, a needle, and optionally instructions for administration, a drug label, contraindications, warnings, or other relevant information.

A terminal sterilization process may be used to sterilize the syringe and such a process may use a known process such as an ethylene oxide or a hydrogen peroxide (H₂O₂) sterilization process. Needles to be used with the syringe may be sterilised by the same method, as may kits described herein. In one aspect, a package is exposed to the sterilising gas until the outside of the syringe is sterile. Following such a process, the outer surface of the syringe may remain sterile (whilst in its blister pack) for up to 6 months, 9 months, 12 months, 15 months, 18 months, 24 months or longer. Thus, in one embodiment, a pre-filed syringe as described herein (in its blister pack) may have a shelf life of up to 6 months, 9 months, 12 months, 15 months, 18 months, 24 months, or even longer. In one embodiment, less than one syringe in a million has detectable microbial presence on the outside of the syringe after 18 months of storage. In one aspect, the pre-filled syringe has been sterilised using ethylene oxide with a Sterility Assurance Level of at least 10⁻⁶. In another aspect, the pre-filled syringe has been sterilised using hydrogen peroxide with a Sterility Assurance Level of at least 10⁻⁶. Significant amounts of the sterilising gas should not enter the variable volume chamber of the syringe. The term “significant amounts” As used herein, refers to an amount of gas that would cause unacceptable modification of the compound solution or suspension within the variable volume chamber. In one embodiment, the pre-filled syringe has been sterilised using ethylene oxide, but the outer surface of the syringe has ≤1 ppm, preferably ≤0.2 ppm ethylene oxide residue. In one embodiment, the pre-filled syringe has been sterilised using hydrogen peroxide, but the outer surface of the syringe has ≤1 ppm, preferably ≤0.2 ppm hydrogen peroxide residue. In another embodiment, the pre-filled syringe has been sterilised using ethylene oxide, and the total ethylene oxide residue found on the outside of the syringe and inside of the blister pack is ≤0.1 mg. In another embodiment, the pre-filled syringe has been sterilised using hydrogen peroxide, and the total hydrogen peroxide residue found on the outside of the syringe and inside of the blister pack is ≤0.1 mg.

Another aspect is a kit of parts. For liquid and suspension compositions, and when the administration device is simply a hypodermic syringe, the kit may comprise the syringe, a needle and a container comprising the compounds described herein for use with the syringe. In case of a dry composition, the container may have one chamber containing the dry compound, and a second chamber comprising a reconstitution solution. In one embodiment, the injection device is a hypodermic syringe adapted so the separate container with compound can engage with the injection device such that in use the liquid or suspension or reconstituted dry composition in the container is in fluid connection with the outlet of the injection device. Examples of administration devices include but are not limited to hypodermic syringes and pen injector devices. Particularly preferred injection devices are the pen injectors, in which case the container is a cartridge, preferably a disposable cartridge.

Another embodiment comprises a kit comprising a needle and a container containing the compounds described herein and optionally further containing a reconstitution solution, the container being adapted for use with the needle. In one aspect, the container is a pre-filled syringe. In another aspect, the container is dual chambered syringe. Another embodiment is a cartridge containing a composition of the compounds described herein for use with a pen injector device. The cartridge may contain a single dose or plurality of doses of drug delivery system.

In another embodiment composition comprises a compound described herein and one or more excipients, and also other biologically active agents, either in their free form or as drugs or combined with other drug delivery systems such as pegylated drugs or hydrogel linked drugs. In one aspect, such additional one or more biologically active agents is a free form drug or a second drug delivery system. In another embodiment, one or more compounds described herein are simultaneously administered, with each compound having either separate or related biological activities or targets.

In an alternative embodiment, the compound is combined with a second biologically active compound in such way that the composition is administered to a subject in need thereof first, followed by the administration of the second compound. Alternatively, the compounds described herein are administered to a subject in need thereof after another compound has been administered to the same subject.

It will be apparent to one of ordinary skill in the relevant art that suitable modifications and adaptations to the compositions, formulations, methods, processes, and applications described herein can be made without departing from the scope of any embodiments or aspects thereof. The compositions, methods, and experiments provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. All patents and publications cited herein are incorporated by reference herein for the specific teachings thereof.

The present disclosure includes duplicates. Duplicates are intended to be supplementary and additive.

### EXAMPLES

### Example 1

### Synthesis of R₁ Groups

The following amines were purchased from commercial sources:

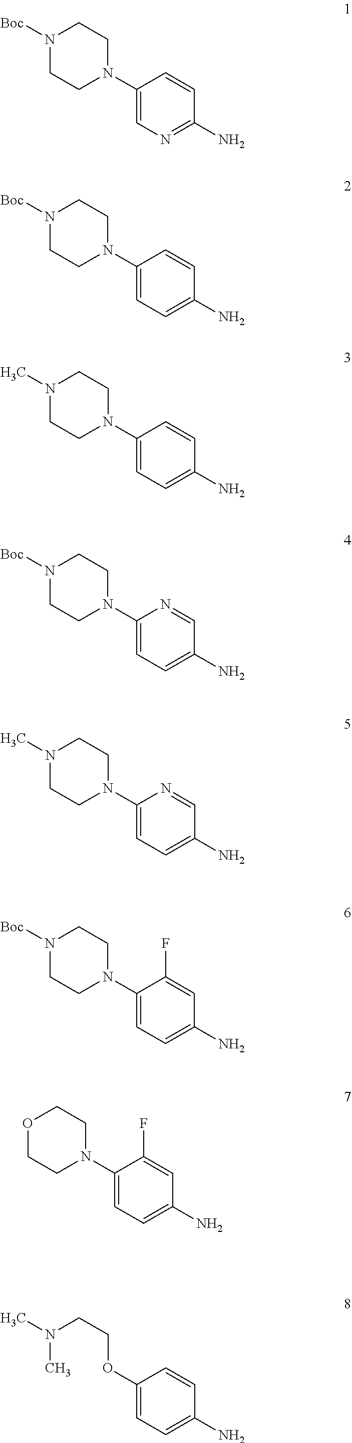

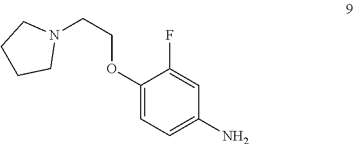 [SMILES: Nc1ccc(OCCN2CCCC2)c(F)c1]

The following amines were synthesized using reported methods.

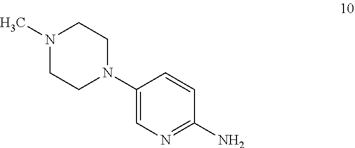

Compound 10 was synthesized using the procedure reported in Int. Pat. App. Publication No. WO 2009156284.

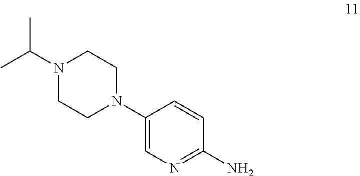 [SMILES: CC(C)N1CCN(c2ccc(N)nc2)CC1]

Compound 11 was synthesized using the reported procedures reported in Int. Pat. App. Publication No. WO 2011112995 and U.S. Pat. No. 7,713,994.

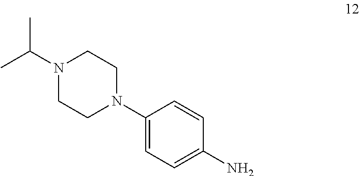 [SMILES: CC(C)N1CCN(c2ccc(N)cc2)CC1]

Compound 12 was synthesized using the procedure reported in Int. Pat. App. Publication No. WO 2007131991.

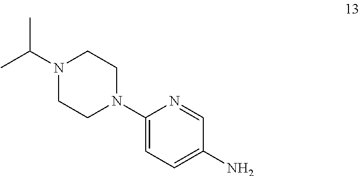 [SMILES: CC(C)N1CCN(c2ccc(N)cn2)CC1]

Compound 13 was synthesized using the procedure reported by Li et al., ACS Med. Chem. Lett., 4(7): 647-650 (2013).

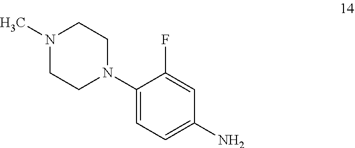

Compound 14 was synthesized using the procedures reported in Int. Pat. App. Publication No. WO 2014012360 and Tsou et al., J. Med. Chem. 51(12): 3507-3525 (2008).

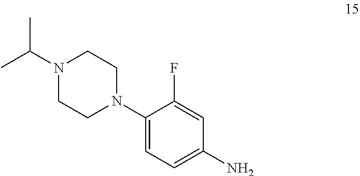 [SMILES: CC(C)N1CCN(c2ccc(N)cc2F)CC1]

Compound 15 was synthesized using the procedures reported in Int. Pat. App. Publication No. WO 2014012360 and Tsou et al., J. Med. Chem. 51(12): 3507-3525 (2008).

### Synthesis of 2-fluoro-4-(4-methylpiperazin-1-yl)aniline (16)

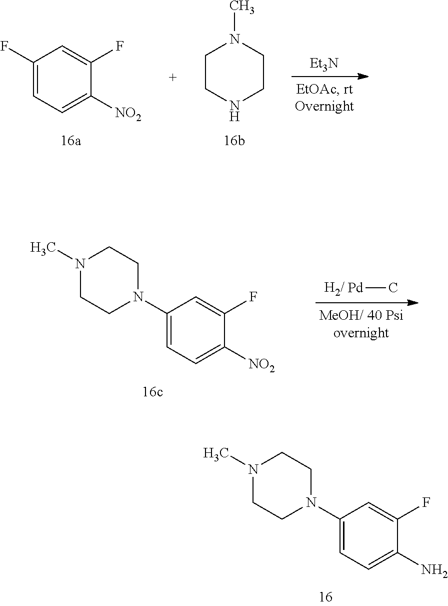

Step 1: Synthesis of 16c: To a solution of 16a (1.4 g, 13.9 mmol) and Et₃N (1.4 g, 13.9 mmol) in EtOAc (15 mL) was added 1,3-difluoro-4-nitrobenzene 16a (2 g, 12.6 mmol) dropwise with stirring and the mixture was stirred at RT overnight. The reaction mixture was diluted with EtOAc (100 mL), washed with brine, dried over anhydrous Na₂SO₄ and concentrated in vacuo to give the title compound 16c as a yellow solid (3.0 g, quantitative). This product is used as such in the next step.

Step 2: Synthesis of 16: To a solution of Compound 16c (3 g, 12.9 mmol) in methanol (100 mL) was added catalytic amount of 10% Pd/C-50% wet (2 g). The mixture was hydrogenated on a Parr apparatus at 35-40 psi for 3 h at RT. The reaction mixture was filtered through a pad of Celite and the filtrate was concentrated under reduced pressure to afford 16 as a solid (2.4 g, 78% yield). MS(ESI) m/z 210.2 [M+H]⁺. ¹H NMR (600 MHz, DMSO-d₆) δ 6.71 (dd, J=10.7, 2.5 Hz, 1H), 6.65-6.61 (m, 2H), 4.53 (s, 2H), 2.80 (s, 4H), 2.52-2.49 (m, 2H), 2.23 (s, 3H).

### Synthesis of 2-fluoro-4-(4-isopropylpiperazin-1-yl)aniline Hydrochloride (17)

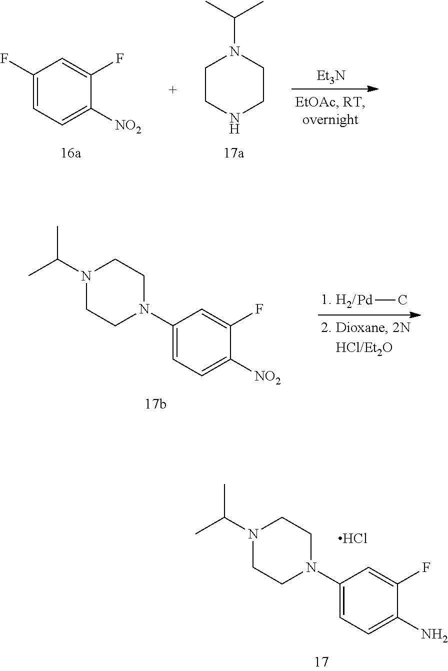

Step 1: Synthesis of 17b: Prepared according to the procedure described for compound 16c (quantitative yield).

Step 1: Synthesis of 17: 30 g (112 mmol) of 17b was hydrogenated following the procedure described for the preparation of compound 16. The resultant crude amine was dissolved in dioxane and treated with 2 N HCl in Et₂O. After stirring for 1 h at RT, the solid is filtered and dried to give 12 g (34%) of 17 as white solid. MS(ESI) m/z 238.2 [M+H]⁺. ¹H NMR (600 MHz, Chloroform-d) δ 6.74 (dt, J=10.2, 1.6 Hz, 1H), 6.63 (dd, J=6.9, 1.6 Hz, 2H), 3.78 (s, 2H), 2.93 (s, 4H), 2.67 (s, 3H), 1.09 (d, J=6.6 Hz, 6H).

### Synthesis of 6-(2-(dimethylamino)ethoxy)pyridin-3-amine (18)

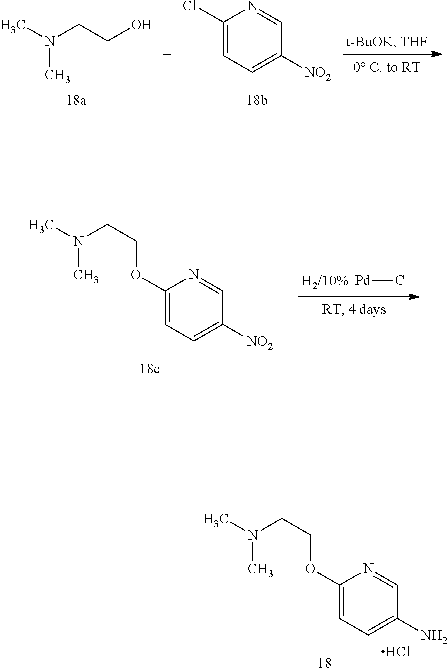

Step 1: Synthesis of 18c: To a solution of 2-(dimethylamino)ethanol 18a (18.55 g, 208 mmol) in THF (200 mL) was added potassium tert-butoxide (18.6 g, 166.6 mmol) portion wise at 0° C. After stirring at room temperature for 1.5 h, 2-chloro-5-nitropyridine 18b (29.8 g, 187.2 mmol) was added in portion wise 0° C. The reaction was warmed to room temperature and stirred for 16 h. The reaction mixture was concentrated under reduced pressure, diluted with water (250 mL), and then extracted with Dichloromethane (3×250 mL). The organic extracts were combined, washed with water, brine, dried over (Na₂SO₄) and concentrated to afford crude material which was further purified by column chromatography on silica gel using 0-7% methanol/methylene chloride to give 2-(5-nitropyridin-2-yloxy)-N, N-dimethylethanamine 18 as a light-yellow solid (20 g, 58%, purity 80%). MS(ESI) m/z 212.2 (M+H)⁺.

Step 1: Synthesis of 18: To a solution of Compound 18c (20 g, 96.1 mmol) in a 1:2 mixture (200 mL) of ethanol and ethyl acetate, catalytic amount of 10% palladium on carbon-50% H₂O (10 g) was added carefully under an argon atmosphere. The flask was flushed three times with hydrogen (standard hydrogenation procedure) and then stirred at room temperature under a hydrogen atmosphere (balloon of H₂ was connected) for 4 days. The reaction mixture was filtered through a pad of Celite and washed with EtOAc. The filtrate was concentrated under reduced pressure to afford the crude product (15 g) as a brown oil. Purification by column chromatography on silica gel using 0-20% percent methanol/methylene chloride (containing 0.25% NH₃ in MeOH) gave 2-(dimethylamino)ethoxy]pyridin-3-amine 18 as a syrup (5.3 g, 31%). MS(ESI) m/z 182.2 [M+H]⁺. ¹H NMR (600 MHz, DMSO-d₆) δ 11.01 (s, 2H), 8.31 (d, J=2.5 Hz, 1H), 7.86 (dd, J=8.8, 2.6 Hz, 1H), 7.03 (d, J=8.8 Hz, 1H), 4.72-4.60 (m, 2H), 3.56-3.46 (m, 2H), 2.82 (d, J=4.0 Hz, 6H).

LC-MS of this compound showed two peaks. 6-[2-(dimethylamino)ethoxy]pyridin-3-amine was converted to di-Boc derivative using standard reaction conditions. Purification by column chromatography on silica gel using 0-4% percent methanol/methylene chloride and then di de-Boc with methanolic hydrochloride gave ˜3 g of 6[2-(dimethylamino)ethoxy]pyridin-3-amine hydrochloride salt 18.

### Synthesis of 6-(2-(pyrrolidin-1-yl)ethoxy)pyridin-3-amine (19)

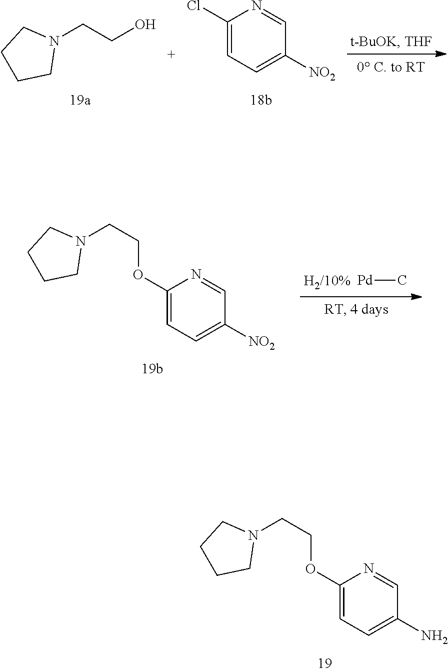

Step 1: Synthesis of 19b: Prepared according to the procedure described for compound 18c (16 g, 32% yield). MS(ESI) m/z 238.2 [M+H]⁺.

Step 1: Synthesis of 19: Prepared according to the procedure described for compound of 18 (4 g, 29% yield). MS(ESI) m/z 208.2 [M+H]⁺. ¹H NMR (600 MHz, Chloroform-d) δ 7.63 (d, J=2.9 Hz, 1H), 7.02 (dd, J=8.7, 3.0 Hz, 1H), 6.63 (d, J=8.7 Hz, 1H), 4.36 (t, J=5.9 Hz, 2H), 3.35 (s, 2H), 2.88 (t, J=5.9 Hz, 2H), 2.68-2.53 (m, 4H), 1.81 (p, J=3.2 Hz, 4H).

### Synthesis of 6-(2-(piperidin-1-yl)ethoxy)pyridin-3-amine (20)

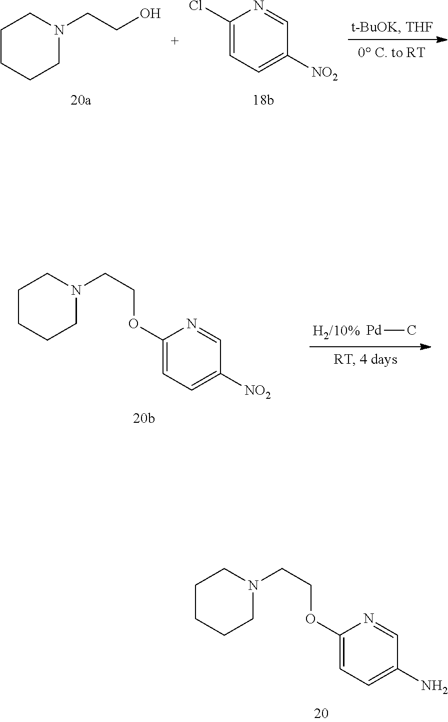

Step 1: Synthesis of 20b: Prepared according to the procedure described for compound 18c (18 g, 34% yield). MS(ESI) m/z 252.2 [M+H]⁺

Step 1: Synthesis of 20: Prepared according to the procedure described for compound 18c (8 g, 51% yield). MS(ESI) m/z 222.2 [M+H]⁺. ¹H NMR (600 MHz, Chloroform-d) δ 7.64 (d, J=2.9 Hz, 1H), 7.01 (dd, J=8.7, 2.9 Hz, 1H), 6.61 (d, J=8.7 Hz, 1H), 4.34 (t, J=6.1 Hz, 2H), 3.36 (s, 2H), 2.73 (t, J=6.1 Hz, 2H), 2.49 (s, 4H), 1.60 (p, J=5.6 Hz, 4H), 1.49-1.38 (m, 2H).

### Synthesis of 5-(2-(dimethylamino)ethoxy)pyridin-2-amine (21)

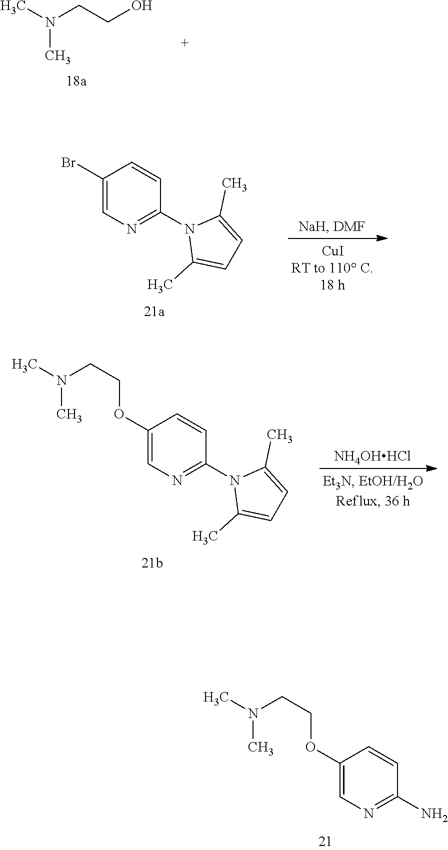

Step 1: Synthesis of 21b: To a solution of 2-(dimethylamino)ethanol 18a (68.8 g, 772 mmol) in DMF (400 mL) was added sodium hydride (60% dispersion in mineral oil) (18.5 g, 454.1 mmol) portion wise at 0° C. The mixture was stirred at room temperature for 1.5 h, cooled to 0° C. again, CuI (7.35 g, 38.6 mmol) and 2-(2,5-dimethyl-1H-pyrrol-1-yl)-5-bromo-pyridine 21a (55 g, 219.0 mmol) were added. The reaction mixture was stirred at 110° C. for overnight. The Reaction mixture was cooled to RT and the solids were filtered over Celite and the filtrate was diluted with water (1.5 lit.) and extracted five times (4×200 mL) with dichloromethane. The combined organic phases were washed with 10% aqueous NH₄OH solution, dried with Na₂SO₄ and concentrated under reduced pressure to yield the crude product which was purified by flash chromatography (silica gel, 0-5% methanol/dichloromethane) to afford the desired product 21 b (45 g, 64%, purity: 70-80%). MS(ESI) m/z 260.3 [M+H]⁺.

Step 2: Synthesis of 21: A mixture of 21b (45 g, 179.2 mmol), hydroxylamine hydrochloride (78.17 g, 1.12 mol), triethylamine (47.11 mL, 335.3 mmol), ethanol (350 mL) and water (150 mL) was refluxed for 36 h. The solution was cooled to RT and the solvent was removed under reduced pressure. The pH was adjusted to 9-10 with 6 M NaOH solution and the resulting mixture was extracted (4×200 mL) with dichloromethane. The combined organic phases were dried over Na₂SO₄, filtered and the solvent was removed in vacuo. The oily residue was purified by column chromatography on silica gel (gradient elution: dichloromethane/methanol) to give 5-(2-(dimethylamino)ethoxy)pyridin-2-amine 21 (6 g, 19%). MS(ESI) m/z 182.2 [M+H]⁺. ¹H NMR (600 MHz, Chloroform-d) δ 7.79 (d, J=2.9 Hz, 1H), 7.12 (dd, J=8.8, 3.0 Hz, 1H), 6.47 (d, J=8.8 Hz, 1H), 4.21 (bs, 2H), 4.01 (t, J=5.7 Hz, 2H), 2.70 (t, J=5.7 Hz, 2H), 2.33 (s, 6H).

### Synthesis of 5-(2-(pyrrolidin-1-yl)ethoxy)pyridin-2-amine (22)

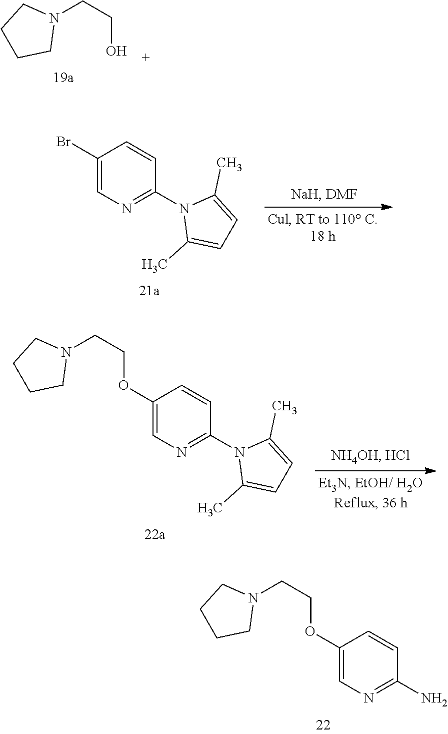 [SMILES: OCCN1CCCC1 ; Cc1ccc(C)n1-c1ccc(Br)cn1 ; Cc1ccc(C)n1-c1ccc(OCCN2CCCC2)cn1 ; Nc1ccc(OCCN2CCCC2)cn1]

Step 1: Synthesis of 22a: Prepared according to the procedure described for compound 21 b (2 g, 67%).

Step 2: Synthesis of 22: Prepared according to the procedure described for compound 21 (6 g, 33% yield). MS(ESI) m/z 208.2 [M+H]⁺. ¹H NMR (600 MHz, Chloroform-d) δ 7.79 (d, J=2.9 Hz, 1H), 7.12 (dd, J=8.8, 2.9 Hz, 1H), 6.47 (d, J=8.8 Hz, 1H), 4.23 (s, 2H), 4.05 (t, J=5.9 Hz, 2H), 2.86 (t, J=5.9 Hz, 2H), 2.66-2.57 (m, 4H), 1.81 (p, J=3.1 Hz, 4H).

### Synthesis of 5-(2-(piperidin-1-yl)ethoxy)pyridin-2-amine (23)

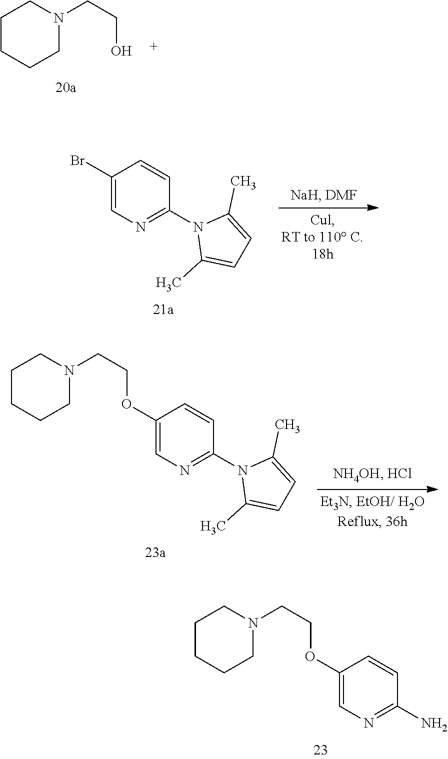 [SMILES: OCCN1CCCCC1 ; Cc1ccc(C)n1-c1ccc(Br)cn1 ; Cc1ccc(C)n1-c1ccc(OCCN2CCCCC2)cn1 ; Nc1ccc(OCCN2CCCCC2)cn1]

Step 1: Synthesis of 23a: Prepared according to the procedure described for compound 21b (45 g, 81% yield).

Step 1: Synthesis of 23: Prepared according to the procedure described for compound 21 (8.5 g, 38% yield). MS(ESI) m/z 222.2 [M+H]⁺. ¹H NMR (600 MHz, Chloroform-d) δ 7.78 (d, J=2.9 Hz, 1H), 7.10 (dd, J=8.8, 2.9 Hz, 1H), 6.46 (d, J=8.8 Hz, 1H), 4.25 (s, 2H), 4.04 (t, J=6.0 Hz, 2H), 2.72 (t, J=6.0 Hz, 2H), 2.48 (s, 4H), 1.60 (p, J=5.6 Hz, 4H), 1.50-1.38 (m, 2H).

### Synthesis of tert-butyl 6-amino-3′,6′-dihydro-[3,4′-bipyridine]-1′(2′H)-carboxylate (24)

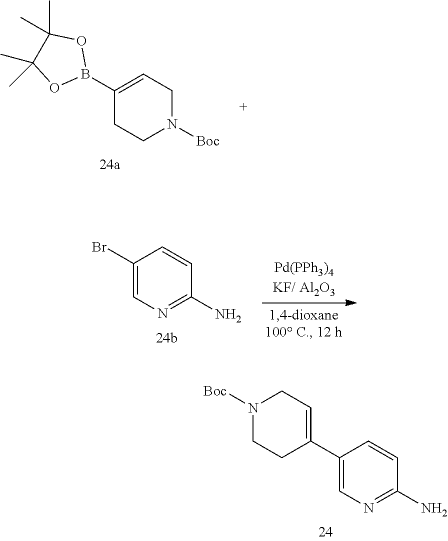 [SMILES: CC(C)(C)OC(=O)N1CC=C(B2OC(C)(C)C(C)(C)O2)CC1 ; Nc1ccc(Br)cn1 ; CC(C)(C)OC(=O)N1CC=C(c2ccc(N)nc2)CC1]

Step 1: Synthesis of 24: To a mixture of 5-bromo-2-aminopyridine 24b (100 g, 0.578 mol) and 4-(4,4,5,5-tetramethyl-[1,3,2]dioxaborolan-2-yl)-3,6-dihydro-2H-pyridine-1-carboxylic acid tertbutylester 24a (121.3 g, 0.578 mol) in 1,4-dioxane (2 L) were added Pd(PPh₃)₄(10 g) and KF/Al₂O₃(200 g, 1:1 ratio) The mixture was degassed for 0.5 h and then heated at 100° C. for 12 h under argon. The reaction mixture was concentrated, and the crude was diluted with EtOAc (2 L), filtered through a pad of Celite, and the filter cake was rinsed with EtOAc (2×250 mL). The combined filtrates were dried over Na₂SO₄, filtered, and concentrated in vacuo. Purification by SiO₂ column chromatography (EtOAc/n-hexanes) provided 24 (35 g, 22% yield) as a brownish solid. MS(ESI) m/z 276.2 [M+H]⁺. ¹H NMR (600 MHz, Chloroform-d) δ 8.08 (s, 1H), 7.47 (dd, J=8.6, 2.3 Hz, 1H), 6.48 (d, J=8.6 Hz, 1H), 5.90 (s, 1H), 4.54 (s, 2H), 4.05 (s, 2H), 3.68-3.53 (m, 2H), 2.45 (s, 2H), 1.49 (s, 9H).

### Synthesis of 1′-isopropyl-1′,2′,3′,6′-tetrahydro-[3,4′-bipyridin]-6-amine (25)

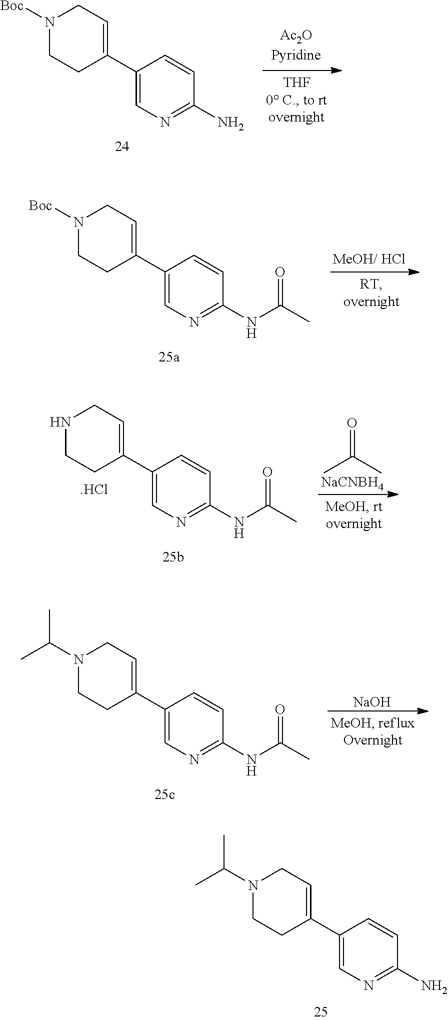

Step 1: Synthesis of 25a: To a solution of tert-butyl 6-amino-3′,6′-dihydro-[3,4′-bipyridine]-1′(2′H)-carboxylate 24 (10 g, 36.2 mmol) and 5 mL pyridine in THF (100 mL) was added acetic anhydride (4.4 g, 43.6 mmol) dropwise at 0° C. The mixture was stirred at room temperature for overnight. The solvent was removed, reaction mixture partitioned between water (50 mL) and dichloromethane (100 mL). The two layers were separated, and the aqueous layer further extracted with dichloromethane (2×100 mL). The organic extracts were combined, washed with brine, dried over sodium sulfate, and concentrated to give the crude material as a brown syrup. Purification by column chromatography on silica gel using 0-3% methanol/methylene chloride yielded 25a as a light-yellow solid (9.1 g, purity 80%). MS(ESI) m/z 318.2 [M+H]⁺.

Step 2: Synthesis of 25b: tert-butyl 6-acetamido-3′,6′-dihydro-[3,4′-bipyridine]-1′(2′H)-carboxylate 25a (9.12 g, 14.3 mmol) was dissolved in 3 N methanolic hydrochloride (200 mL) and stirred for overnight. The reaction mixture was concentrated to afford crude N-(1′,2′,3′,6′-tetrahydro-[3,4′-bipyridin]-6-yl)acetamide hydrochloride salt, 25b (8.2 g).

Step 3: Synthesis of 25c: A solution of N-(1′,2′,3′,6′-tetrahydro-[3,4′-bipyridin]-6-y1) acetamide hydrochloride 25b (4.0 g, 15.8 mmol) in 50 mL of methanol was added to a mixture of acetone (2 mL) and sodium cyanoborohydride (3.92 g, 63.2 mmol) and the resultant mixture was stirred at room temperature for overnight. The reaction mixture was concentrated and evaporated to dryness. The residue was partitioned between dichloromethane (100 mL) and saturated sodium bicarbonate (50 mL). The layers were separated, and the aqueous layer was extracted with methylene chloride (4×100 mL). The organic phases were combined and washed with brine, dried (Na₂SO4) and concentrated. The residue was purified by column chromatography (dichloromethane:methanol=95:5, v/v) to afford of 25c as a light-yellow syrup (2.1 g, 53% yield).

Step 4: Synthesis of 25: To a solution of compound 25c (1.8 g, 6.9 mmol) in MeOH (100 mL), NaOH (2.78 g, 69 mmol) was added and the resultant mixture was stirred at reflux temperature for 18 h. The standard work-up and chromatography purification (see Preston et al., Dalton Transactions, 45(19): 8050-8060 (2016)) afforded compound 25 (0.888 g, 60%) as a light-yellow solid. MS(ESI) m/z 218.2 [M+H]⁺. ¹H NMR (600 MHz, DMSO-d₆) δ 7.95 (s, 1H), 7.46 (d, J=8.6 Hz, 1H), 6.40 (d, J=8.6 Hz, 1H), 5.93 (s, 1H), 5.89 (s, 2H), 3.09 (s, 2H), 2.77-2.65 (m, 1H), 2.65-2.54 (m, 2H), 2.35 (s, 2H), 1.01 (d, J=6.4 Hz, 6H).

### Synthesis of tert-butyl 6-(4-amino-2-fluorophenyl)-2,6-diazaspiro[3.3]heptane-2-carboxylate (26)

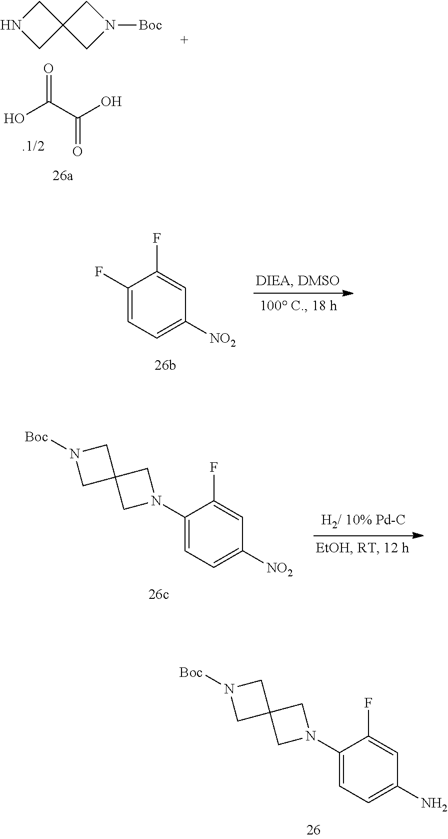

Step 1: Synthesis of 26c: To a mixture of tert-butyl 2,6-diazaspiro[3.3]heptane-2-carboxylate hemioxalate 26a (1.5 g, 6.17 mmol) and DIEA (3.3 mL, 19 mmol) in DMSO (15 mL) was added 1,3-difluoro-4-nitrobenzene 26b (1 g, 6.3 mmol) dropwise over a period of 5 minutes. The resultant reaction mixture was stirred at 100° C. for 18 h and then cooled to room temperature. The reaction mixture was and then poured into H₂O (100 mL) and extracted with EtOAc (3×50 mL). Th combined organic layer was washed with water (2×25 mL), brine (1×25 mL), dried (Na₂SO₄) and concentrated to afford the product 26c as a yellow solid (˜1.8 g) which was used as such for the next step.

Step 2: Synthesis of 26: The Parr-hydrogenator flask (500 mL) was charged with nitro compound 26c (1.8 g, 5.6 mmol) and ethanol (100 mL). A 10% Pd—C (1 g, 50% H₂O) was introduced carefully under argon. The resultant mixture was hydrogenated at ˜33 Psi 12 h at RT. The reaction mixture was filtered through pad of Celite and washed with EtOAc. The filtrate was concentrated under reduced pressure to afford the title product 26 as a gray solid (1.36 g 72% yield). MS(ESI) m/z 308.2 [M+H]⁺. ¹H NMR (600 MHz, Chloroform-d) δ 6.43-6.29 (m, 3H), 4.07 (s, 4H), 3.92 (d, J=1.5 Hz, 4H), 1.44 (s, 9H).

### Synthesis of tert-butyl 6-(5-aminopyridin-2-yl)-2,6-diazaspiro[3.3]heptane-2-carboxylate (27)

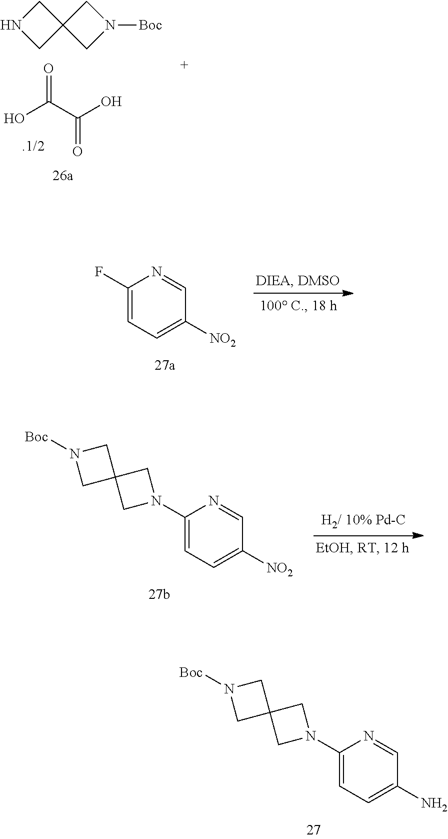

Step 1: Synthesis of 27b: Prepared according to the procedure described for compound 26c.

Step 2: Synthesis of 27: Prepared according to the procedure described for compound 26 (1.36 g, 76% yield). MS(ESI) m/z 291.2 [M+H]⁺. ¹H NMR (600 MHz, Chloroform-d) δ 7.73 (d, J=2.6 Hz, 1H), 6.97 (dd, J=8.6, 2.7 Hz, 1H), 6.24 (d, J=8.6 Hz, 1H), 4.08 (s, 4H), 4.02 (s, 4H), 1.44 (s, 9H).

### Synthesis of tert-butyl 6-(4-aminophenyl)-2,6-diazaspiro[3.3]heptane-2-carboxylate (28)

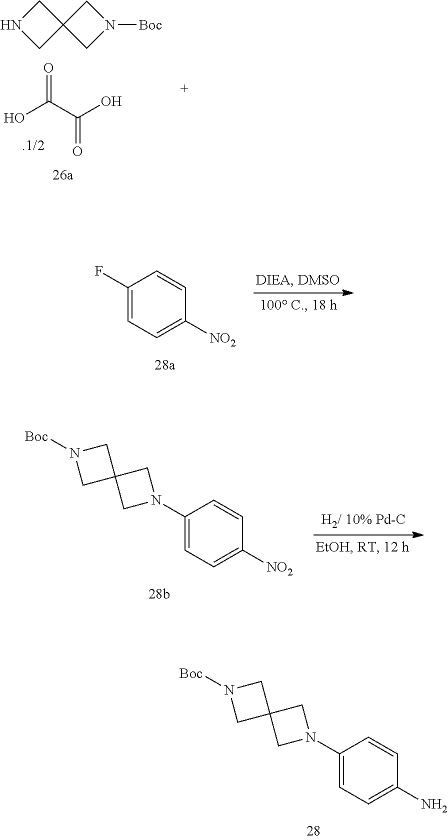

Step 1: Synthesis of 28b: Prepared according to the procedure described for compound 26c.

Step 2: Synthesis of 28: Prepared according to the procedure described for compound 26 (7.12 g, 98% yield). MS(ESI) m/z 290.2 [M+H]⁺. ¹H NMR (600 MHz, Chloroform-d) δ 6.63 (d, J=8.5 Hz, 2H), 6.35 (d, J=8.5 Hz, 2H), 3.33 (S, 2H), 4.06 (s, 4H), 1.44 (s, 9H).

### Synthesis of tert-butyl 4-(4-amino-3-fluorophenyl)piperazine-1-carboxylate (29)

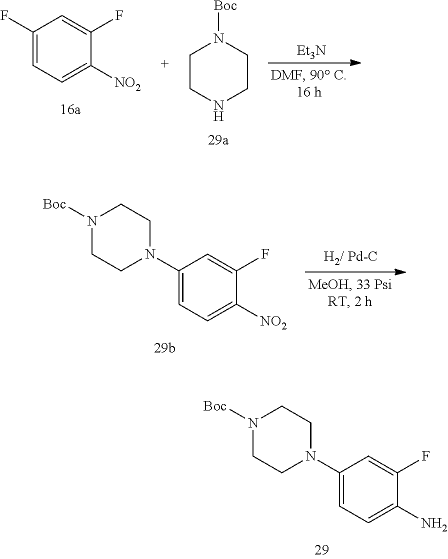 [SMILES: "Et3N" ; O=[N+]([O-])c1ccc(F)cc1F ; CC(C)(C)OC(=O)N1CCNCC1 ; CC(C)(C)OC(=O)N1CCN(c2ccc([N+](=O)[O-])c(F)c2)CC1 ; CC(C)(C)OC(=O)N1CCN(c2ccc(N)c(F)c2)CC1]

Step 1: Synthesis of 29b: (Ref: US 20090093497): A mixture of 2,4-difluoro-nitro-benzene 16a (20 g, 126 mmol), piperazine-1-carboxylic acid tert-butyl ester 29a (23.4 g, 126 mmol), and triethylamine (63 mL, 378 mmol) in anhydrous DMF (200 mL) was stirred at 90° C. for 16 h. The reaction mixture was partitioned between water and ethyl acetate, layers were separated, and the aqueous layer was extracted with ethyl acetate twice. Organic layers were collected, combined, washed with brine, dried over sodium sulfate, filtered, and concentrated. The residue was purified by silica-gel column with ethyl acetate and hexanes as eluting solvents to give the product 29b as a light-yellow solid (25.1 g, 62% yield). MS(ESI) m/z 326.1 [M+H]⁺.

Synthesis of 29: (Ref: WO 2015038417): The solution of compound 29b (25 g, 89.2 mmol) in Methanol (300 mL) in the presence of 10% palladium on carbon (12.5 g, 50% wet) was shaken under the hydrogen with a pressure of 33 psi at room temperature for 12 hr. The reaction mixture was filtered through a plug of Celite and the filtration pad was washed with methanol. The organic layer was collected, concentrated, and dried to give compound 29 (26 g, 99%) as a light red gummy material, which was used as such without further purification. MS(ESI) m/z 296.2 [M+H]⁺. ¹H NMR (600 MHz, Chloroform-d) δ 6.74-6.70 (m, 1H), 6.66-6.63 (m, 1H), 6.59-6.55 (m, 1H), 3.56 (t, J=5.1 Hz, 4H), 2.96 (d, J=5.6 Hz, 4H), 1.48 (s, 9H).

### Synthesis of (S)-1-(4-amino-2-fluorophenyl)-N,N-dimethylpyrrolidin-3-amine (30)

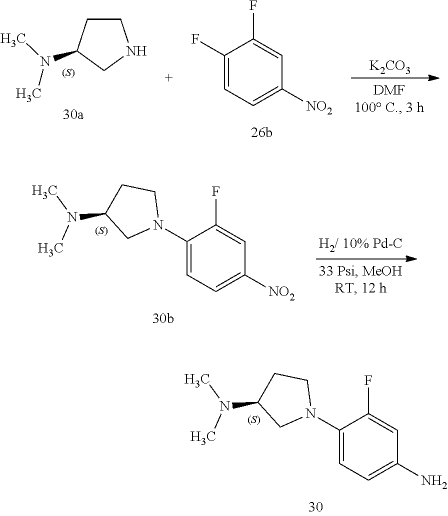

Step 1: Synthesis of 30b: (Ref: WO 2015038417): To a stirred mixture of compound 30a (2.0 g, 16.9 mmol) in DMF (50 mL) at room temperature were added 26b (3.0 g, 18.9 mmol) and K₂CO3 (13.1 g, 94.5 mmol). The resulting mixture was stirred at 110° C. for 16 h. After cooling down to room temperature, the reaction mixture was diluted with water (200 mL) and extracted with EtOAc (3×150 mL). The organic layer was washed with brine solution (50 mL), dried (Na₂SO₄), and concentrated to provide crude residue. The crude was triturated with n-hexane and filtered to give compound 30b as a yellow solid (3.8 g, 80% yield). MS(ESI) m/z 254.2 [M+H]⁺.

Step 2: Synthesis of 30: (Ref: WO 2015038417): The solution of compound 30b (3.75 g 15 mmol) in Methanol (200 mL) in the presence of 10% palladium on carbon (2 g, 50% H₂O) was shaken under the hydrogen with a pressure of 33 psi at room temperature for 12 hr. The reaction mixture was filtered through a plug of Celite and the filtration pad was washed with methanol. The organic layer was collected, concentrated, and dried to give compound 30 (3.2 g, 96%) as a light red solid, which was directly used in the next step reaction without further purification. MS(ESI) m/z 224.2 [M+H]⁺. ¹H NMR (600 MHz, Chloroform-d) δ 6.62-6.52 (m, 1H), 6.49-6.34 (m, 2H), 3.49-3.31 (m, 4H), 3.31-3.18 (m, 1H), 3.18-3.09 (m, 1H), 2.83 (p, J=7.6 Hz, 1H), 2.28 (s, 6H), 2.21-2.07 (m, 1H), 1.92-1.80 (m, 1H).

### Synthesis of (S)-6-(3-(dimethylamino)pyrrolidin-1-yl)pyridin-3-amine (31)

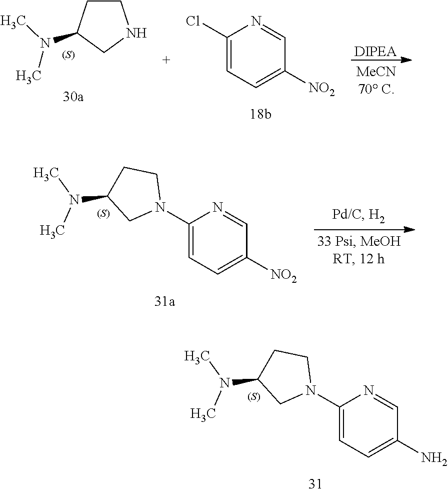

Step 1: Synthesis of 31a: (Ref: WO 20050261307): To a stirred mixture of compound 30a (1.5 g, 12.5 mmol) in MeCN (25 mL) at room temperature, nitro compound 18b (1.8 g, 11.4 mmol) and diisopropylethylamine (4 mL, 23 mmol) were added and stirred at 70° C. for overnight. The reaction mixture was concentrated under reduced pressure and the residue was dissolved in EtOAc (150 mL) and then washed with H₂O (2×30 mL), brine, dried over sodium sulfate and concentrated to give 31a as yellow solid (2.3 g, 85% yield). MS(ESI) m/z 237.2 [M+H]⁺.

Step 2: Synthesis of 31: Prepared according to the procedure described for compound 30 (1.85 g, 92%). MS(ESI) m/z 207.2 [M+H]⁺. ¹H NMR (600 MHz, DMSO-d₆) δ 7.60 (d, J=2.6 Hz, 1H), 7.34 (s, 2H), 7.01 (dd, J=8.7, 2.7 Hz, 1H), 6.40 (d, J=8.8 Hz, 1H), 3.85 (q, J=7.4 Hz, 1H), 3.69 (dd, J=10.6, 7.6 Hz, 1H), 3.55-3.47 (m, 2H), 3.24 (q, J=8.5 Hz, 2H), 2.73 (s, 6H), 2.38-2.30 (m, 1H), 2.29-2.20 (m, 1H).

### Synthesis of (S)-5-(3-(dimethylamino)pyrrolidin-1-yl)pyridin-2-amine (32)

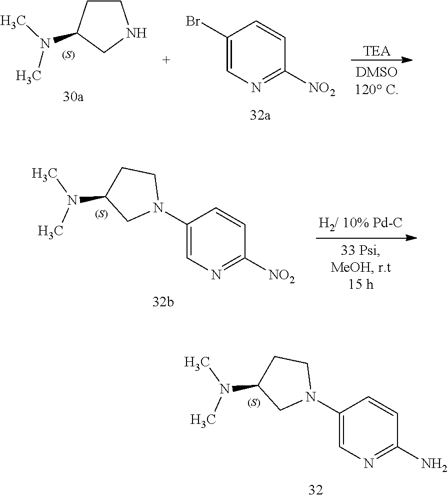

Step 1: Synthesis of 32b: (Ref: WO 20180072707): To a stirred mixture of compound 30a (1.5 g, 12.5 mmol) and nitro compound 32a (1.8 g, 11.4 mmol) in DMSO (15 mL) was added triethylamine (4 mL, 23 mmol) at room temperature and stirred at 120° C. for overnight. The reaction mixture was diluted with water (200 mL) and extracted with EtOAc (3×150 mL). The organic layer was washed with brine solution (50 mL), dried (Na₂SO₄), and concentrated to provide crude residue. The crude residue was further purified by silica-gel column using 0-20% MeOH in DCM to give 32b as yellow solid (3.3 g, 80% yield). MS(ESI) m/z 237.2 [M+H]⁺.

Step 2: Synthesis of 32: Prepared according to the procedure described for compound 30 (2.8 g, quantitative yield). MS(ESI) m/z 207.2 [M+H]⁺. ¹H NMR (600 MHz, Chloroform-d) δ 7.48 (d, J=2.9 Hz, 1H), 6.84 (dd, J=8.8, 3.0 Hz, 1H), 6.50 (d, J=8.7 Hz, 1H), 3.43-3.37 (m, 1H), 3.37-3.30 (m, 1H), 3.30-3.24 (m, 1H), 3.10 (t, J=8.2 Hz, 1H), 2.91-2.84 (m, 1H), 2.31 (s, 6H), 2.23-2.15 (m, 1H), 1.97-1.88 (m, 1H).

### Synthesis of (S)-1-(4-aminophenyl)-N,N-dimethylpyrrolidin-3-amine (33)

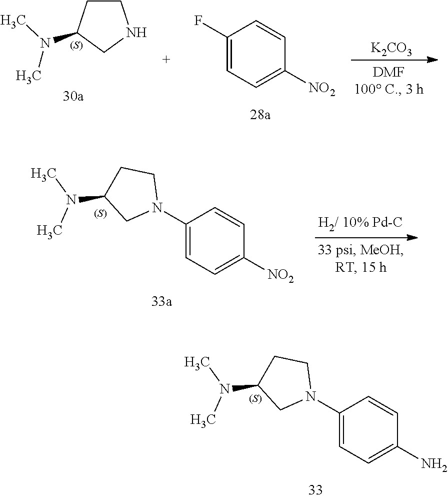

Step 1: Synthesis of 33a: Prepared according to the procedure described for compound 30b. (3.8 g, 81% yield) as yellow solid. MS(ESI) m/z 236.2 [M+H]⁺.

Step 1: Synthesis of 33: Prepared according to the procedure described for compound 30 (3.23 g, 99% yield). MS(ESI) m/z 206.2 [M+H]⁺. ¹H NMR (600 MHz, Chloroform-d) δ 6.67 (d, J=8.6 Hz, 2H), 6.45 (d, J=8.6 Hz, 2H), 3.41 (t, J=7.9 Hz, 1H), 3.38-3.23 (m, 2H), 3.09 (t, J=8.2 Hz, 1H), 2.89-2.80 (m, 1H), 2.30 (s, 6H), 2.22-2.13 (m, 1H), 1.97-1.84 (m, 1H).

### Synthesis of 3-fluoro-4-(4-morpholinopiperidin-1-yl)aniline (34)

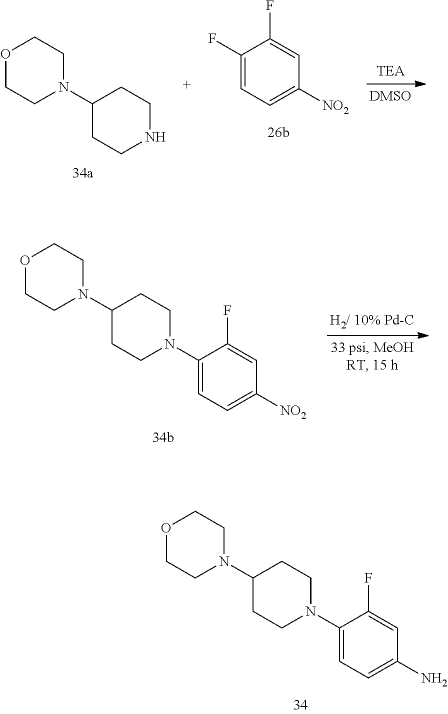 [SMILES: C1CC(N2CCOCC2)CCN1 ; O=[N+]([O-])c1ccc(F)c(F)c1 ; O=[N+]([O-])c1ccc(N2CCC(N3CCOCC3)CC2)c(F)c1 ; Nc1ccc(N2CCC(N3CCOCC3)CC2)c(F)c1]

Step 1: Synthesis of 34b: (Ref: WO 20160400858): To a stirred mixture of compound 34a (4.0 g, 23.5 mmol) and compound 26b (3.4 g, 21.4 mmol) in DMSO (20 mL) at room temperature was added triethylamine (9.8 mL, 71 mmol) and stirred at 120° C. for overnight. After TLC showed completion of starting material the mixture was diluted with water (100 mL) and the solid formed was filtered and dried under vacuum to give the product 34b (6.3 g, 84% yield) as yellow solid which is used in the next step without further purification. MS(ESI) m/z 310.1 [M+H]⁺.

Step 1: Synthesis of 34: (Ref: WO 2015038417): The solution of compound 34b (6.3 g, 0.4 mmol) in Methanol (mL) in the presence of 10% palladium on carbon was shaken under the hydrogen with a pressure of 50 psi at room temperature for 2 hr. The reaction mixture was filtered through a plug of Celite and the filtration pad was washed with methanol. The organic layer was collected, concentrated, and dried to give compound 34 (4.9 g, 86%) as a light red solid, which was directly used in the next step reaction without further purification. MS(ESI) m/z 280.2 [M+H]⁺. ¹H NMR (600 MHz, Chloroform-d) δ 6.80 (t, J=8.9 Hz, 1H), 6.49-6.34 (m, 2H), 3.77-3.69 (m, 3H), 3.53 (s, 2H), 3.34 (d, J=11.6 Hz, 2H), 2.67-2.53 (m, 5H), 2.38-2.27 (m, 1H), 1.94-1.85 (m, 2H), 1.81-1.61 (m, 3H).

### Synthesis of 6-(4-morpholinopiperidin-1-yl)pyridin-3-amine (35)

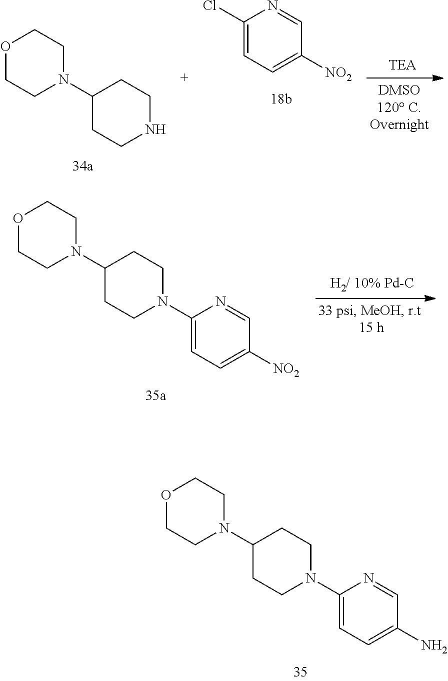 [SMILES: C1CC(N2CCOCC2)CCN1 ; O=[N+]([O-])c1ccc(Cl)nc1 ; O=[N+]([O-])c1ccc(N2CCC(N3CCOCC3)CC2)nc1 ; Nc1ccc(N2CCC(N3CCOCC3)CC2)nc1]

Synthesis of 35a: Prepared according to the procedure described for compound 34b (3.6 g, 69% yield). MS(ESI) m/z 293.2 [M+H]⁺.

Synthesis of 35: Prepared according to the procedure described for compound 34 (1.25 g, 40% yield). MS(ESI) m/z 263.2 [M+H]⁺. ¹H NMR (600 MHz, Chloroform-d) δ 7.78 (d, J=2.7 Hz, 1H), 6.97 (dd, J=8.8, 2.9 Hz, 1H), 6.60 (d, J=8.8 Hz, 1H), 4.10 (d, J=12.8 Hz, 2H), 3.75-3.71 (m, 3H), 3.28 (s, 2H), 2.73 (td, J=12.6, 2.0 Hz, 2H), 2.64-2.51 (m, 4H), 2.41-2.30 (m, 1H), 1.92 (d, J=12.5 Hz, 2H), 1.64-1.48 (m, 2H).

### Synthesis of 5-(4-morpholinopiperidin-1-yl)pyridin-2-amine (36)

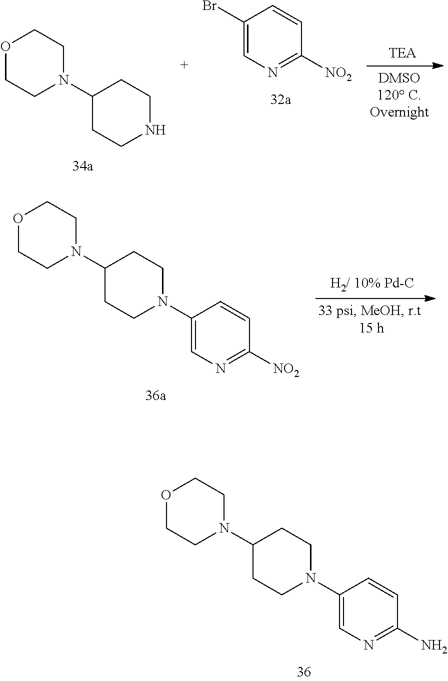 [SMILES: C1CC(N2CCOCC2)CCN1 ; O=[N+]([O-])c1ccc(Br)cn1 ; O=[N+]([O-])c1ccc(N2CCC(N3CCOCC3)CC2)cn1 ; Nc1ccc(N2CCC(N3CCOCC3)CC2)cn1]

Synthesis of 36a: Prepared according to the procedure described for compound 34b (3.5 g, 68% yield). MS(ESI) m/z 293.2 [M+H]⁺.

Synthesis of 37: Prepared according to the procedure described for compound 34 (1.25 g, 40% yield). MS(ESI) m/z 263.2 [M+H]⁺. ¹H NMR (600 MHz, Chloroform-d) δ 7.78 (d, J=2.8 Hz, 1H), 7.18 (dd, J=8.8, 2.9 Hz, 1H), 6.47 (d, J=8.8 Hz, 1H), 3.76-3.72 (m, 5H), 3.47 (d, J=12.3 Hz, 2H), 2.67-2.61 (m, 2H), 2.61-2.56 (m, 4H), 2.32-2.26 (m, 1H), 1.94 (d, J=12.5 Hz, 2H), 1.72-1.63 (m, 2H).

### Synthesis of 4-(4-morpholinopiperidin-1-yl)aniline (37)

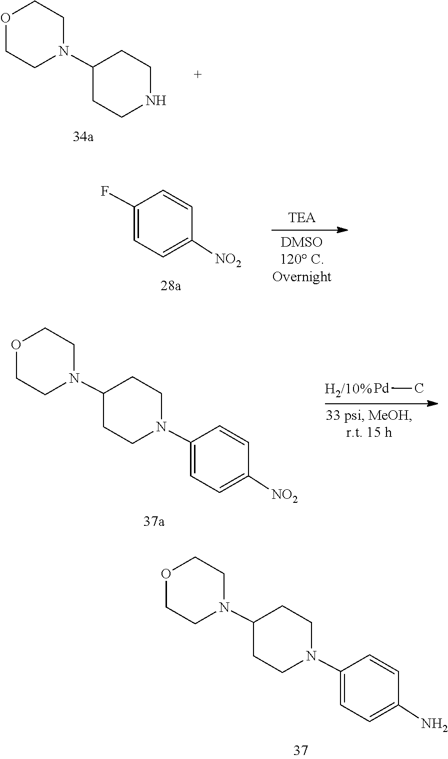

Synthesis of 37a: Prepared according to the procedure described for compound 34b (3.6 g, 70% yield). MS(ESI) m/z 292.2 [M+H]⁺.

Synthesis of 37: Prepared according to the procedure described for compound 34 (1.25 g, 40% yield). MS(ESI) m/z 262.2 [M+H]⁺. ¹H NMR (600 MHz, Chloroform-d) δ 6.86-6.78 (m, 2H), 6.70-6.58 (m, 2H), 3.77-3.68 (m, 3H), 3.52 (d, J=12.3 Hz, 2H), 3.42 (s, 2H), 2.63-2.60 (m, 1H), 2.60-2.56 (m, 4H), 2.34-2.24 (m, 1H), 1.96-1.88 (m, 2H), 1.77-1.62 (m, 3H).

### Synthesis of (S)-(6-aminopyridin-3-yl)(3-(dimethylamino)pyrrolidin-1-yl)methanone (38)

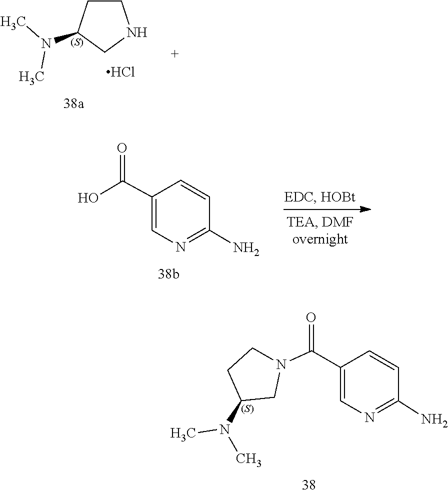

Synthesis of 38: (Ref: WO 2016015605): To a solution of compound 38b (1.1 g, 8.0 mmol) in DMF (25 mL) were added EDC (1.53 g, 8 mmol), HOBt (1.22 g, 8 mmol) and triethylamine (5.5 mL, 40 mmol). After stirring the mixture for 10 min, compound 38a (1.5 g, 8 mmol) was added. The resulting mixture was stirred at room temperature for overnight. The reaction mixture was concentrated in vacuo and the residue was purified by silica-gel chromatography using 0-10% MeOH in DCM to give 38 as a light-yellow solid (0.61 g, 34% yield). MS(ESI) m/z 235.2 [M+H]⁺. ¹H NMR (600 MHz, Chloroform-d) δ 8.31 (s, 1H), 7.74-7.63 (m, 1H), 6.49 (d, J=8.5 Hz, 1H), 4.81 (s, 2H), 3.82-3.54 (m, 2H), 3.45-3.37 (m, 1H), 2.79-2.61 (m, 1H), 2.26 (d, J=40.6 Hz, 6H), 2.20-2.05 (m, 2H), 1.88-1.79 (m, 1H).

### Synthesis of (S)-(5-aminopyridin-2-yl)(3-(dimethylamino)pyrrolidin-1-yl)methanone (39)

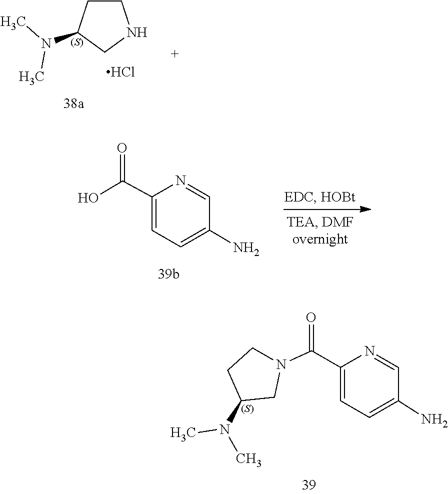

Synthesis of 39: Prepared according to the procedure described for compound 38 (0.95 g, 50% yield). MS(ESI) m/z 235.2 [M+H]⁺. ¹H NMR (600 MHz, Chloroform-d) δ 8.00 (dd, J=11.5, 2.7 Hz, 1H), 7.75 (d, J=8.4 Hz, 1H), 7.00 (dd, J=8.5, 2.7 Hz, 1H), 4.14-4.10 (m, 0.5H), 4.05-4.00 (m, 0.5H), 3.98 (d, J=12.2 Hz, 2H), 3.91-3.83 (m, 1H), 3.65-3.59 (m, 1H), 3.45-3.38 (m, 1H), 2.76-2.67 (m, 1H), 2.30 (s, 3H), 2.26 (s, 3H), 2.16-2.08 (m, 1H), 1.87-1.74 (m, 3H).

### Synthesis of (S)-(4-amino-2-fluorophenyl)(3-(dimethylamino)pyrrolidin-1-yl)methanone (40)

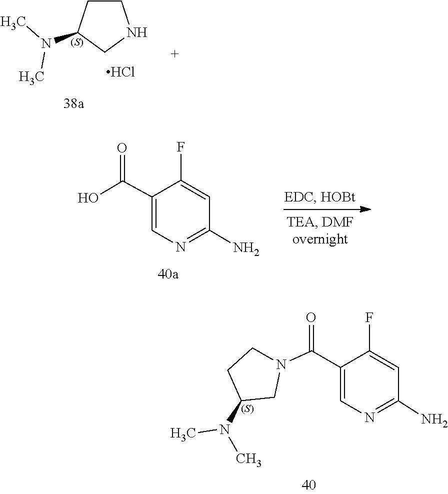

Synthesis of 40: Prepared according to the procedure described for compound 38 (1.0 g, 50% yield). MS(ESI) m/z 252.2 [M+H]⁺. ¹H NMR (600 MHz, Chloroform-d) δ 7.22-7.15 (m, 1H), 6.46-6.40 (m, 1H), 6.35-6.28 (m, 1H), 4.15 (d, J=14.1 Hz, 2H), 3.94-3.86 (m, 0.5H), 3.84-3.75 (m, 0.5H), 3.63-3.40 (m, 2H), 3.40-3.33 (m, 0.5H), 3.33-3.24 (m, 0.5H), 2.81-2.71 (m, 0.5H), 2.72-2.64 (m, 0.5H), 2.28 (s, 3H), 2.21 (s, 3H), 2.19-2.12 (m, 0.5H), 2.10-2.02 (m, 0.5H), 1.87-1.73 (m, 1H).

### Synthesis of (S)-(4-aminophenyl)(3-(dimethylamino)pyrrolidin-1-yl)methanone (41)

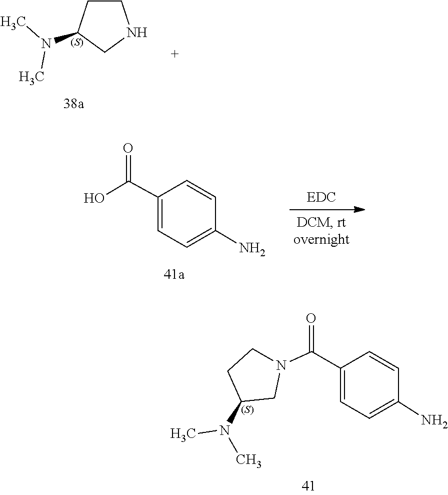

Synthesis of 41: Prepared according to the procedure described for compound 38 (0.63 g, 45% yield). MS(ESI) m/z 234.2 [M+H]⁺. ¹H NMR (600 MHz, Chloroform-d) δ 7.39 (d, J=8.0 Hz, 2H), 6.64 (d, J=8.3 Hz, 2H), 3.89 (s, 3H), 3.83-3.50 (m, 3H), 3.45-3.30 (m, 1H), 2.68 (d, J=68.0 Hz, 1H), 2.26 (d, J=44.2 Hz, 6H), 1.83-1.77 (m, 1H).

### Synthesis of 4-(4-isopropylpiperazin-1-yl)-3-methoxyaniline (42)

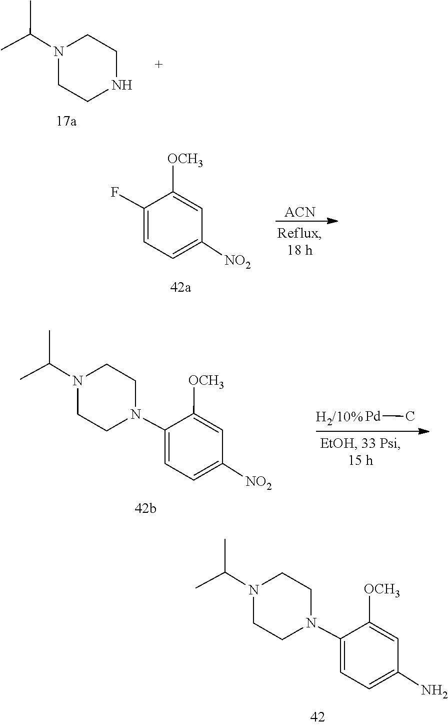

Following the above scheme, the title compound 42 (4.75 g, 64% for two steps) was synthesized. MS(ESI) m/z 250.2 [M+H]⁺. ¹H NMR (600 MHz, Chloroform-d) δ 6.78 (d, J=8.2 Hz, 1H), 6.31-6.15 (m, 2H), 3.81 (s, 3H), 3.48 (s, 2H), 3.01 (s, 4H), 2.81-2.65 (m, 5H), 1.10 (d, J=6.5 Hz, 6H).

### Synthesis of 4-(4-isopropylpiperazin-1-yl)-3-methylaniline (43)

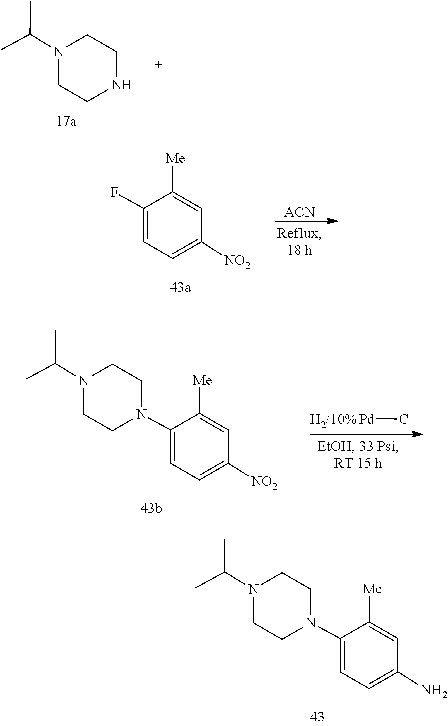

Following the scheme, the title compound 43, (4.46 g, 64% for two steps), was synthesized. MS(ESI) m/z 234.3 [M+H]⁺. ¹H NMR (600 MHz, Chloroform-d) δ 6.89 (d, J=8.3 Hz, 1H), 6.55 (d, J=2.6 Hz, 1H), 6.50 (dd, J=8.3, 2.7 Hz, 1H), 3.44 (s, 2H), 2.91-2.81 (m, 4H), 2.75-2.67 (m, 2H), 2.65 (s, 3H), 2.23 (s, 3H), 1.09 (d, J=6.5 Hz, 6H).

The amines 44, 45, and 46 were prepared by following the reported methods. See Walia et al., J. Org. Chem. 78(21): 10931-10937 (2013).

### Synthesis of 5-(4-isopropylpiperazin-1-yl)-4-(trifluoromethyl)pyridin-2-amine (44)

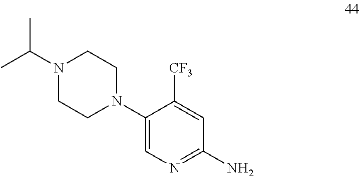 [SMILES: CC(C)N1CCN(c2cnc(N)cc2C(F)(F)F)CC1]

MS(ESI) m/z 289.3 [M+H]⁺.

### Synthesis of 5-(4-methylpiperazin-1-yl)-4-(trifluoromethyl)pyridin-2-amine (45)

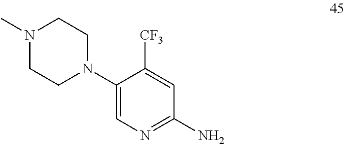 [SMILES: CN1CCN(c2cnc(N)cc2C(F)(F)F)CC1]

MS(ESI) m/z 261.3 [M+H]⁺.

### Synthesis of tert-butyl 4-(6-amino-4-(trifluoromethyl)pyridin-3-yl)piperazine-1-carboxylate (46)

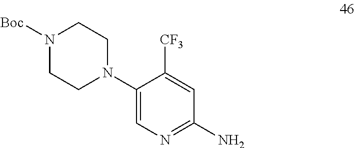 [SMILES: CC(C)(C)OC(=O)N1CCN(c2cnc(N)cc2C(F)(F)F)CC1]

MS(ESI) m/z 347.3 [M+H]⁺.

### Synthesis of 5-(1,4-diazabicyclo[3.2.2]nonan-4-yl)pyridin-2-amine (47)

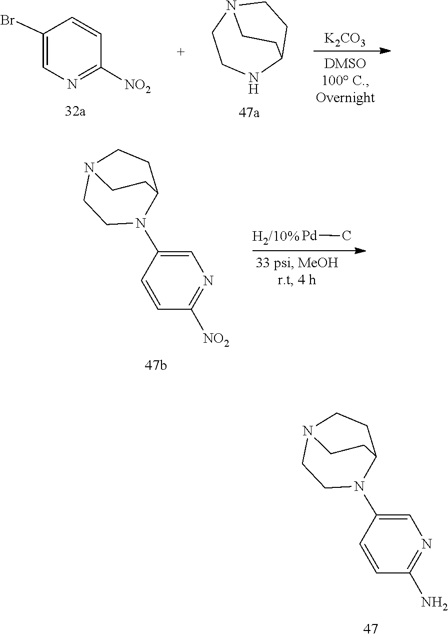

Step 1: Synthesis of 47b: Prepared according to the procedure described for the preparation of compound 30b (12 g, 52% yield).

Step 2: Synthesis of 47: Prepared according to the procedure described for the preparation of compound 32 (4 g, 66% yield). MS(ESI) m/z 219.3 [M+H]⁺.

Compounds 48 and 49 were purchased from commercial sources:

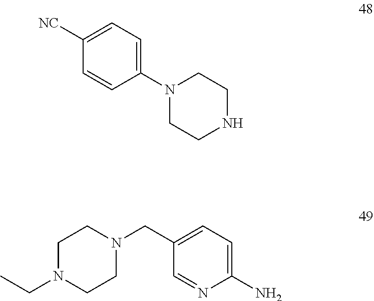 [SMILES: N#Cc1ccc(N2CCNCC2)cc1 ; CCN1CCN(Cc2ccc(N)nc2)CC1]

The Following Amines were Synthesized Using a Reported Procedure:

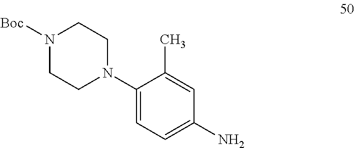 [SMILES: Cc1cc(N)ccc1N1CCN(C(=O)OC(C)(C)C)CC1]

Compound 50 was synthesized using procedure reported in WO2001068643.

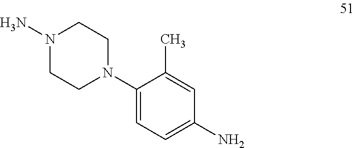

Compound 51 was synthesized using procedure reported in WO2012059932.

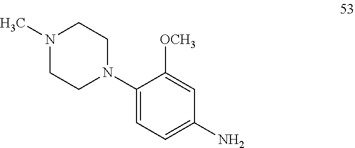 [SMILES: COc1cc(N)ccc1N1CCN(C)CC1]

Compound 52 was synthesized using procedure reported in WO2008051547.

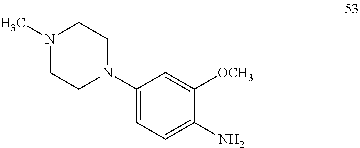 [SMILES: COc1cc(N2CCN(C)CC2)ccc1N]

Compound 53 was synthesized using procedure reported in WO2017076355 and US20180208564

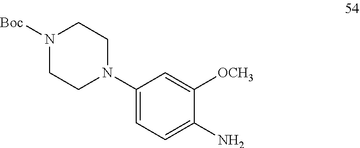 [SMILES: COc1cc(N2CCN(C(=O)OC(C)(C)C)CC2)ccc1N]

Compound 54 was synthesized using procedure reported in WO2017143842 and JMC 2019, 62, 4401-4410

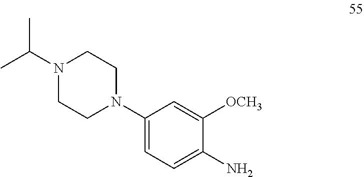 [SMILES: COc1cc(N2CCN(C(C)C)CC2)ccc1N]

Compound 55 was synthesized using procedure reported in WO2004080980

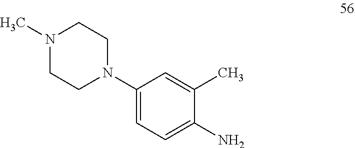

Compound 56 was synthesized using procedure reported in WO2008051547

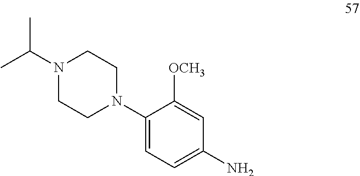 [SMILES: COc1cc(N)ccc1N1CCN(C(C)C)CC1]

Compound 57 was synthesized using procedure reported in WO2005009443

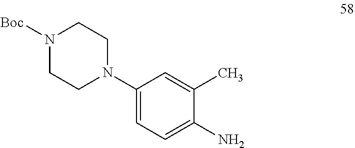 [SMILES: Cc1cc(N2CCN(C(=O)OC(C)(C)C)CC2)ccc1N]

Compound 58 was synthesized using procedure reported in WO2007089768 and WO2006004200

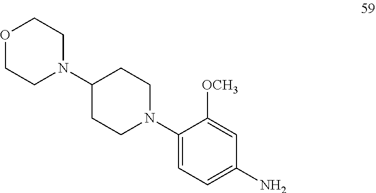 [SMILES: COc1cc(N)ccc1N1CCC(N2CCOCC2)CC1]

Compound 59 was synthesized using procedure reported in WO2016049211

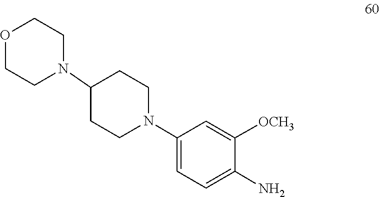 [SMILES: COc1cc(N2CCC(N3CCOCC3)CC2)ccc1N]

Compound 60 was synthesized using procedure reported in WO2018183112

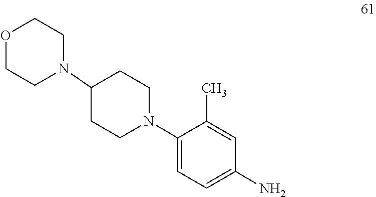 [SMILES: Cc1cc(N)ccc1N1CCC(N2CCOCC2)CC1]

Compound 61 was synthesized using procedure reported in WO2012053606

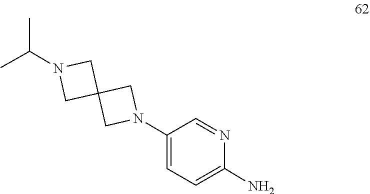 [SMILES: CC(C)N1CC2(CN(c3ccc(N)nc3)C2)C1]

Synthesis of 62: Prepared according to the procedure described for the preparation of compound 91. MS (ESI) m/z 233.2 [M+H]⁺.

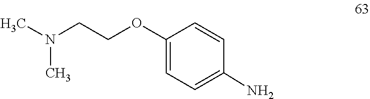

Compound 63 was synthesized using procedure reported in WO2008152013

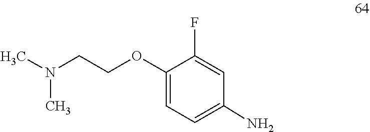

Compound 64 was synthesized using procedure reported in WO2009076140 and BMCL 2015, 23, 1044-1054

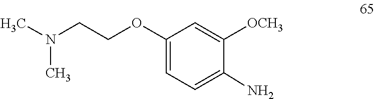

Compound 65 was synthesized using procedure reported in WO2014134308

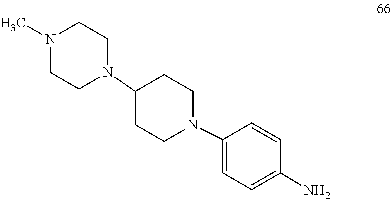 [SMILES: CN1CCN(C2CCN(c3ccc(N)cc3)CC2)CC1]

Compound 66 was synthesized using procedure reported in eJMC 2019, 163, 690-709

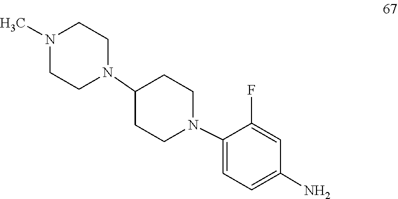 [SMILES: CN1CCN(C2CCN(c3ccc(N)cc3F)CC2)CC1]

Compound 67 was synthesized using procedure reported in WO2012053605

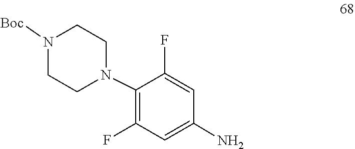 [SMILES: CC(C)(C)OC(=O)N1CCN(c2c(F)cc(N)cc2F)CC1]

Compound 68 was synthesized using procedure reported in WO20120220572

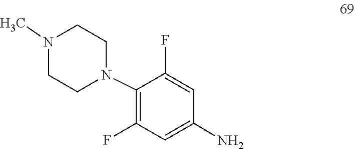 [SMILES: CN1CCN(c2c(F)cc(N)cc2F)CC1]

Compound 69 was synthesized using procedure reported in WO2017066428

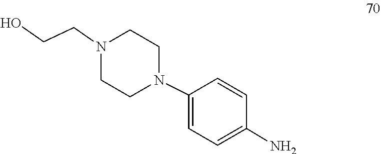 [SMILES: Nc1ccc(N2CCN(CCO)CC2)cc1]

Compound 70 was synthesized using procedure reported in JMC 2005, 48, 2371-2387 and WO2003037872

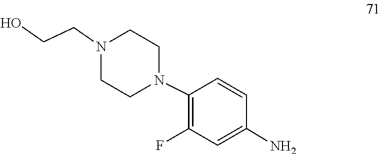 [SMILES: Nc1ccc(N2CCN(CCO)CC2)c(F)c1]

Compound 71 was synthesized using procedure reported in WO2010090764

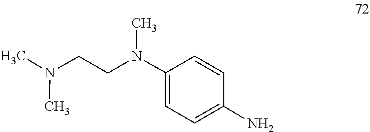

Compound 72 was synthesized using procedure reported in WO2004011438 and WO2008152013

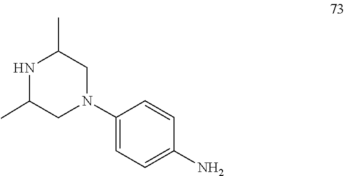

Compound 73 was synthesized using procedure reported in WO2015092431 and JMC 2008, 51, 3507-3525

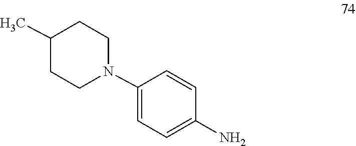

Compound 74 was synthesized using procedure reported in JMC 2012, 55, 10685-10699

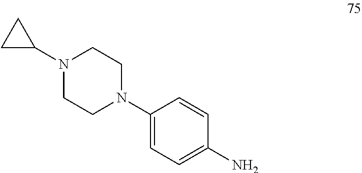 [SMILES: Nc1ccc(N2CCN(C3CC3)CC2)cc1]

Compound 75 was synthesized using procedure reported in WO2017211216

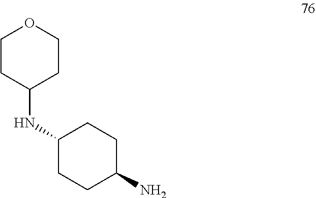 [SMILES: N[C@H]1CC[C@H](NC2CCOCC2)CC1]

Compound 76 was synthesized using procedure reported in WO2012058392

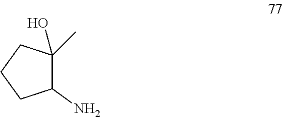

Compound 77 was synthesized using procedure reported in JMC 2016, 59, 750-755

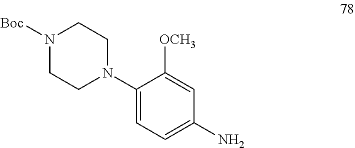 [SMILES: COc1cc(N)ccc1N1CCN(C(=O)OC(C)(C)C)CC1]

Compound 78 was synthesized using procedure reported in Part in WO2009079597 and WO2002010146

### Synthesis of 4-4-(isopropylpiperazin-1-yl)-2-methylaniline (79)

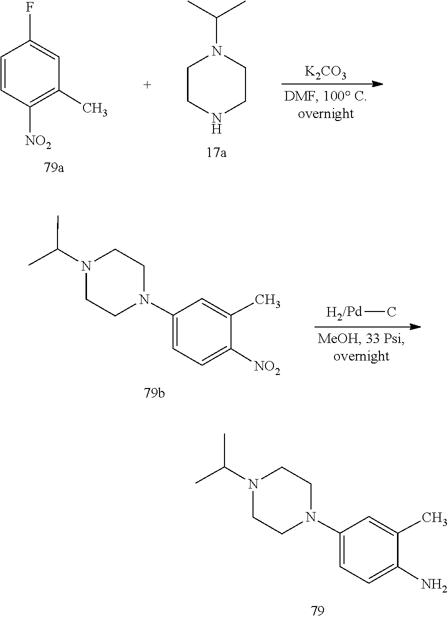

Step 1: Synthesis of 79b: (Part in WO2009079597) To a stirred mixture of compound 79a (3.6 g, 23.4 mmol) in DMF (25 mL) at room temperature were added 17a (3.0 g, 23.4 mmol) and K₂CO3 (9.7 g, 70.2 mmol). The resulting mixture was stirred at 100° C. for overnight. After cooling to room temperature, the reaction mixture was diluted with water (200 mL) and the solid obtained was filtered, washed with water (3×25 mL), and dried under vacuum to give compound 79b as a yellow solid (5.1 g, 75% yield). MS (ESI) m/z 264.2 [M+H]⁺. This product was used as such in the next step.

Step 2: Synthesis of 79: (Part in WO2002010146) To a solution of compound 79b (4.5 g, 17.1 mmol) in methanol (200 mL) was added a 10% Pd/C-50% wet (2.3 g). The mixture was hydrogenated on a par apparatus at 33 psi for overnight at RT. The reaction mixture was filtered through a pad of Celite and the filtrate was concentrated to afford 79 as a solid (3.5 g, 88% yield). MS (ESI) m/z 233.2 [M+H]⁺. ¹H NMR (600 MHz, Chloroform-d) δ 6.73 (d, J=2.8 Hz, 1H), 6.70 (dd, J=8.4, 2.8 Hz, 1H), 6.62 (d, J=8.4 Hz, 1H), 3.09-3.03 (m, 4H), 2.74-2.66 (m, 5H), 2.17 (s, 2H), 2.16 (s, 3H), 1.09 (d, J=6.5 Hz, 6H).

### Synthesis of 5-(1-methyl-1H-pyrazol-4-yl)pyridine-2-amine (80)

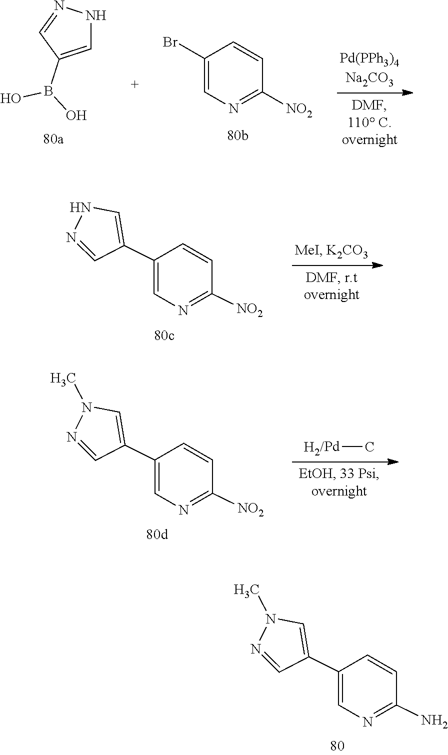

Step 1: Synthesis of 80c: (Part in WO2012082817) To a mixture of compound 80a (5.0 g, 45 mmol) and compound 80b (9.1 g, 45 mmol) in DMF (100 mL) was added 2M Na₂CO₃ solution (10 mL) and the reaction mixture was degassed for 5 min and then tetrakis(triphenylphosphine)palladium(0) (0.52 g, 0.45 mmol) was added at room temperature under Argon. The resulting mixture was stirred at 110° C. for overnight. Reaction mixture was cooled and poured onto H₂O (1000 mL). The solid obtained was filtered and washed with methanol to give compound 80c as a solid (2.1 g, 25% yield). MS (ESI) m/z 191.2 [M+H]⁺

Step 2: Synthesis of 80d: (Part in WO2018124001) To a stirred mixture of compound 80c (1.0 g, 5.3 mmol) in DMF (20 mL) at room temperature were added Mel (1.5 g, 10.5 mmol) and K₂CO3 (2.2 g, 16 mmol). The resulting mixture was stirred at room temperature for overnight. Reaction mixture was cooled and poured onto H₂O (200 mL). The solid obtained was filtered and dried to give compound 80d as a solid (0.75 g, 75% yield). MS (ESI) m/z 205.1 [M+H]⁺.

Step 3: Synthesis of 80: (Part in WO2002010146) Following the standard hydrogenation conditions and isolation techniques gave 80 as a solid (240 mg, 38% yield). MS (ESI) m/z 175.1 [M+H]⁺.

### Synthesis of 5-(1-isopropyll-1H-pyrazol-4-yl)pyridine-2-amine (81)

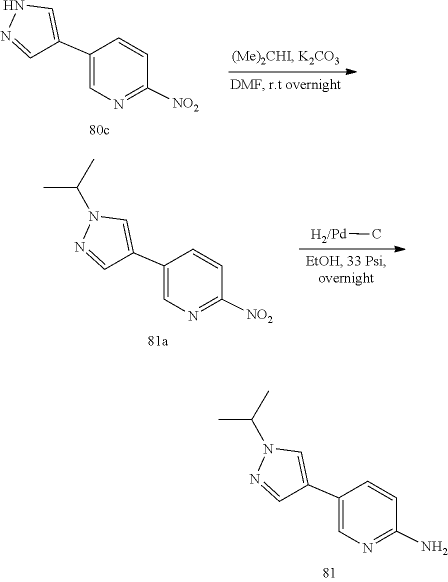

Step 1: Synthesis of 81b: (Part in WO2018124001) To a stirred mixture of compound 80c (1.0 g, 5.3 mmol) in DMF (20 mL) at room temperature were added (Me)₂CH1 (1.8 g, 10.5 mmol) and K₂CO₃ (2.2 g, 16 mmol). The resulting mixture was stirred at room temperature for overnight. The reaction mixture was diluted with water (100 mL) and extracted with EtOAc (3×100 mL). The organic layer was washed with brine solution (50 mL), dried (Na₂SO₄), and concentrated to provide crude residue. The crude residue was further purified by silica-gel column using 0-20% MeOH in DCM to give 81b as a solid (0.9 g, 75% yield). MS (ESI) m/z 233.1 [M+H]⁺.

Step 2: Synthesis of 81: (Part in WO2002010146) Following the standard hydrogenation conditions and isolation techniques gave 81 as a solid (670 mg, 86% yield). MS (ESI) m/z 203.2 [M+H]⁺. 5 H NMR (600 MHz, Chloroform-d) δ 8.22 (dd, J=2.4, 0.8 Hz, 1H), 7.69-7.66 (m, 1H), 7.58 (d, J=0.8 Hz, 1H), 7.54 (dd, J=8.4, 2.4 Hz, 1H), 7.27 (s, 1H), 6.53 (dd, J=8.5, 0.9 Hz, 1H), 4.62-4.47 (m, 1H), 4.41 (s, 2H), 1.81 (s, 1H), 1.54 (d, J=6.7 Hz, 6H).

### Synthesis of 5-(1H-Pyrazol-4-yl)pyridin-2-amine (82)

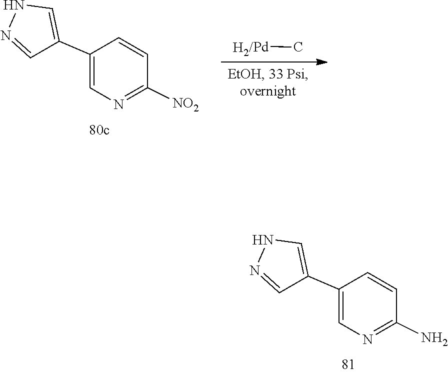

Step 1: Synthesis of 82: (Part in WO2002010146) To a solution of compound 80c (1.1 g, 3.8 mmol) in ethanol (100 mL) was added catalytic amount of 10% Pd/C-50% wet (600 mg). The mixture was hydrogenated on a par apparatus at 33 psi for overnight at RT. Following standard isolation techniques gave 81 as a solid (660 mg, 76% yield). MS (ESI) m/z 161.2 [M+H]⁺.

### Synthesis of tert-butyl-4-(6-aminopheyl)-1H-pyrazole-1carboxylate (82)

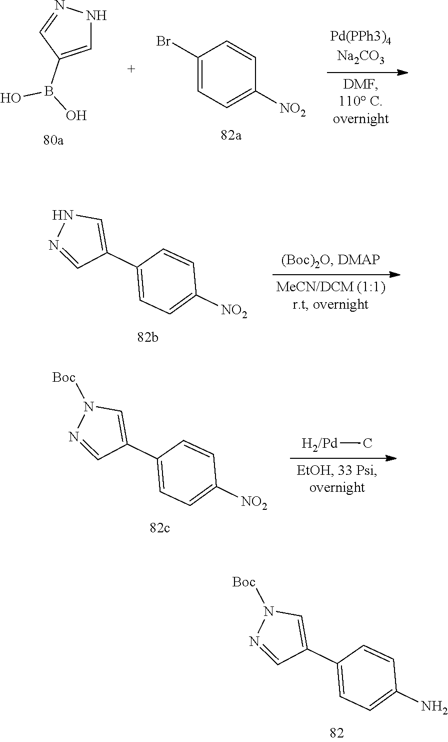

Step 1: Synthesis of 82b: (Part in WO2012082817) The procedure used for making 80c was used to isolate 82b as a solid (3.7 g, 44% yield). MS (ESI) m/z 190.1 [M+H]⁺. Step 2: Synthesis of 82c: (Part in WO2018005193) To a stirred mixture of compound 82b (1.0 g, 5.3 mmol) in MeCN/DCM (1:1) (50 mL) at room temperature were added (Boc)₂O (5.8 g, 27 mmol) and DMAP (324 mg, 2.7 mmol). The resulting mixture was stirred at room temperature for overnight. The work-up and column chromatography gave 82c as a solid (1.1 g, 66% yield). MS (ESI) m/z 291.1 [M+H]⁺.

Step 3: Synthesis of 82: (Part in WO2002010146) The hydrogenation of 82c (1.1 g, 0.8 mmol) in ethanol (100 mL) using catalytic amount of 10% Pd/C-50% wet (600 mg) gave 82 as a solid (950 mg, 96% yield). MS (ESI) m/z 260.1 [M+H]⁺. 6 ¹H NMR (600 MHz, Chloroform-d) δ 8.17 (d, J=0.9 Hz, 1H), 7.91 (d, J=0.9 Hz, 1H), 7.32 (d, J=8.5 Hz, 2H), 6.71 (d, J=8.5 Hz, 2H), 1.67 (s, 9H).

### Synthesis of tert-butyl-4-(1-methyl-1H-pyrazol-4-yl)aniline (83)

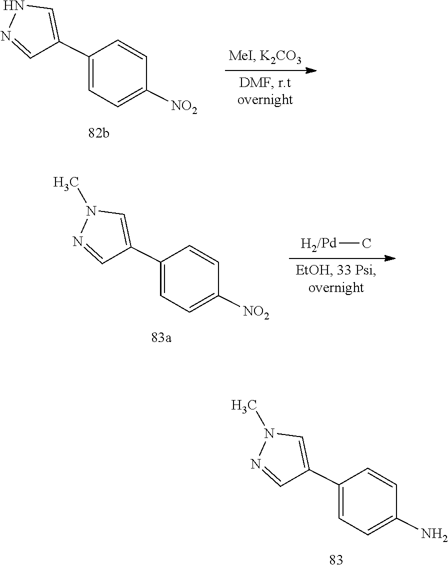

Step 1: Synthesis of 83a: (Part in WO2018124001) To a stirred mixture of compound 82b (1.0 g, 5.3 mmol) in DMF (20 mL) at room temperature were added Mel (1.5 g, 10.5 mmol) and K₂CO3 (2.2 g, 16 mmol). The resulting mixture was stirred at room temperature for overnight. The work-up and purification gave 83a as a solid (0.8 g, 73% yield). MS (ESI) m/z 204.1 [M+H]⁺.

Step 2: Synthesis of 83: (Part in WO2002010146) Following the standard hydrogenation technique and isolation procedure gave 83 as a gray solid (660 mg, 97% yield). MS (ESI) m/z 174.1 [M+H]⁺. 5 ¹H NMR (600 MHz, Chloroform-d) δ 7.66 (s, 1H), 7.49 (s, 1H), 7.28-7.25 (m, 2H), 6.69 (d, J=8.4 Hz, 2H), 3.91 (s, 3H), 3.66 (s, 2H).

### Synthesis of 4-(1-isopropyl-1H-pyrazol-4-yl)aniline (84)

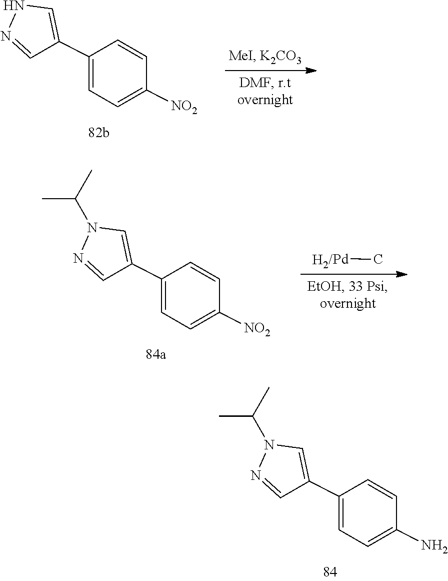

Step 1: Synthesis of 84a: (Part in WO2018124001) The procedure described for 81a was employed to isolate 84a as a solid (0.9 g, 75% yield). MS (ESI) m/z 233.2 [M+H]⁺.

Step 2: Synthesis of 84: (Part in WO2002010146) (750 mg, 96% yield). Following the standard hydrogenation technique and isolation procedure gave 84 as a gray solid MS (ESI) m/z 203.1 [M+H]⁺. ¹H NMR (600 MHz, Chloroform-d) δ 7.68 (s, 1H), 7.55 (s, 1H), 7.27 (dd, J=8.9, 6.9 Hz, 2H), 6.72-6.66 (m, 2H), 4.55-4.46 (m, 1H), 1.53 (d, J=6.7 Hz, 6H).

### Synthesis of 4-(2-(dimethylamino)ethoxy)-3-methoxyaniline (85)

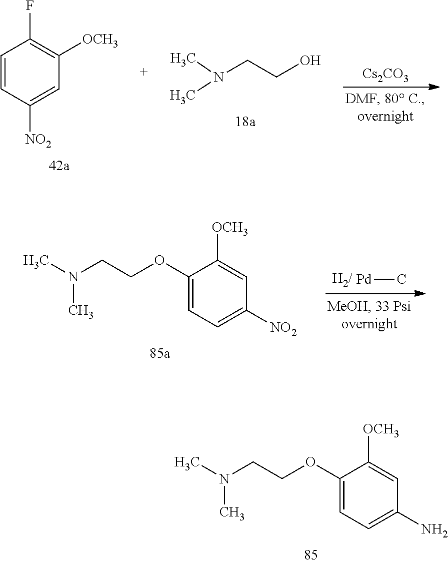

Step 1: Synthesis of 85a: (Part in 2009076140) To a stirred mixture of compound 42a (5.0 g, 29 mmol) in DMF (50 mL) at room temperature were added 18a (3.9 g, 44 mmol) and Cs₂CO₃ (29 g, 188 mmol). The resulting mixture was stirred at 80° C. for overnight. Reaction mixture was cooled, diluted with water (200 mL) and extracted with EtOAc (3×200 mL). The organic layer was washed with brine solution (100 mL), dried (Na₂SO₄), and concentrated to provide crude residue. The crude residue was further purified by silica-gel column using 0-20% MeOH in DCM to give 85a as a solid (5.0 g, 71% yield). MS (ESI) m/z 241.1 [M+H]⁺.

Step 2: Synthesis of 85: (Part in WO2002010146) To a solution of compound 85a (5.0 g, 21 mmol) in methanol (200 mL) was added catalytic amount of 10% Pd/C-50% wet (2.5 g). The mixture was hydrogenated on a par apparatus at 40 psi for overnight at RT. The reaction mixture was filtered and concentrated to afford 85 as a solid (3.9 g, 89% yield). MS (ESI) m/z 211.2 [M+H]+. ¹H NMR (600 MHz, DMSO-d₆) δ 7.95 (s, 1H), 6.64 (d, J=8.4 Hz, 1H), 6.26 (d, J=2.6 Hz, 1H), 6.04 (dd, J=8.4, 2.5 Hz, 1H), 3.84 (t, J=6.1 Hz, 2H), 3.67 (s, 3H), 2.55-2.48 (m, 3H), 2.19 (s, 6H).

### Synthesis of 4-(2-(dimethylamino)ethoxy)-3-methylaniline (86)

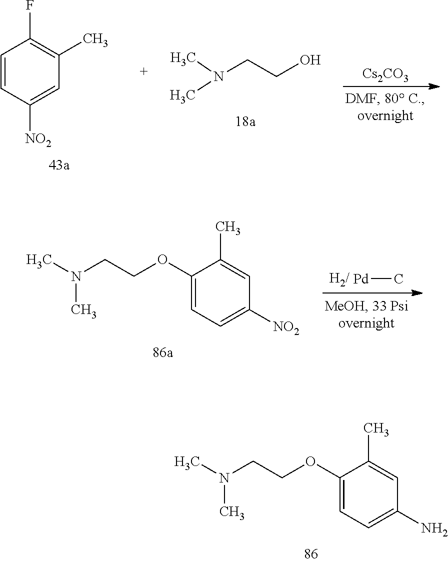

Step 1: Synthesis of 86a: (Part in 2009076140) The procedure to make 85a was used to isolate 86a as a solid (7.0 g, 97% yield). MS (ESI) 225.1 m/z [M+H]⁺.

Step 2: Synthesis of 86: (Part in WO2002010146) The standard hydrogenation technique and isolation procedure was used to isolate 86 as a solid (6.0 g, 99% yield). MS (ESI) m/z 195.2 [M+H]+. 6 ¹H NMR (600 MHz, DMSO-d₆) δ 7.95 (s, 1H), 6.62 (s, 1H), 6.38 (s, 1H), 6.33 (s, 1H), 3.86 (s, 2H), 2.21 (s, 6H), 2.03 (s, 3H).

### Synthesis of (R)-5-(3-(dimethylamino)pyrrolidin-1-yl)pyridin-2-amine (87)

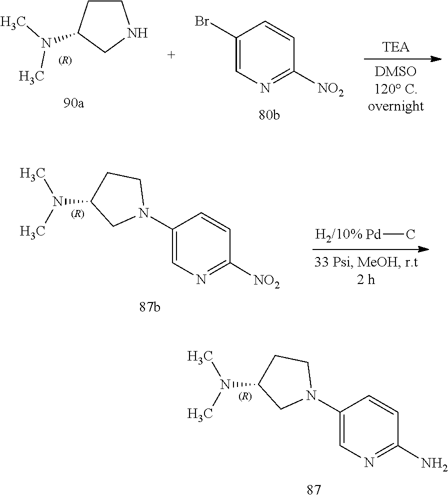

Step 1: Synthesis of 87b: (Part in Ref: WO 20180072707 A1): To a stirred mixture of compound 90a (5.0 g, 44 mmol) and bromo compound 80b (8.4 g, 42 mmol) in DMSO (25 mL) was added triethylamine (31 mL, 220 mmol) at room temperature and stirred at 120° C. for overnight. The reaction mixture was cooled and diluted with water (300 mL) and extracted with EtOAc (3×150 mL). The organic layer was washed with brine solution (100 mL), dried (Na₂SO₄), and concentrated to provide crude residue. The crude residue was further purified by silica-gel column using 0-20% MeOH in DCM to give 87bc as yellow solid (8.0 g, 78% yield). MS (ESI) m/z 237.2 [M+H]⁺.

Step 2: Synthesis of 87: (Part in WO 2015038417 A1) Hydrogenation of 87b by following standard hydrogenation technique and isolation method afforded 90 as a solid (6.8 g, 97% yield). MS (ESI) m/z 207.2 [M+H]⁺. ¹H NMR (600 MHz, Chloroform-d) δ 7.48 (d, J=3.0 Hz, 1H), 6.84 (dd, J=8.8, 3.0 Hz, 1H), 6.50 (d, J=8.7 Hz, 1H), 3.42-3.36 (m, 1H), 3.32 (dd, J=8.8, 2.7 Hz, 1H), 3.30-3.23 (m, 1H), 3.10 (t, J=8.2 Hz, 1H), 2.90-2.84 (m, 1H), 2.31 (s, 6H), 2.23-2.15 (m, 1H), 1.96-1.87 (m, 1H).

### Synthesis of 4-(2-(dimethylamino)ethoxy)-2-methylaniline (88)

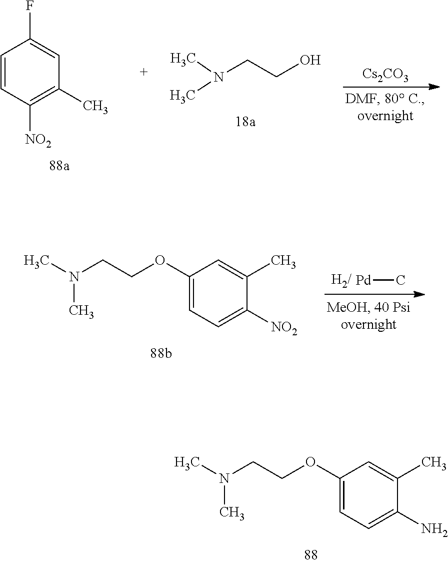

Step 1: Synthesis of 88b: (Part in WO2014134308) The procedure to make 85b was used to isolate 88b as a solid (4.1 g, 57% yield). MS (ESI) m/z 225.2 [M+H]⁺.

Step 2: Synthesis of 88: (Part in WO2002010146) Hydrogenation and then filtered through a pad of Celite and the filtrate was concentrated under reduced pressure to afford 91 as a solid (3.0 g, 83% yield). MS (ESI) m/z 195.1[M+H]⁺. ¹H NMR (600 MHz, Chloroform-d) δ 6.69 (d, J=2.8 Hz, 1H), 6.63 (d, J=2.8 Hz, 1H), 6.61 (s, 1H), 3.99 (t, J=5.8 Hz, 2H), 2.68 (t, J=5.8 Hz, 2H), 2.32 (s, 6H), 2.15 (s, 3H)

### Synthesis of 4-(2-(dimethylamino)ethoxy)-2-fluoroaniline (89)

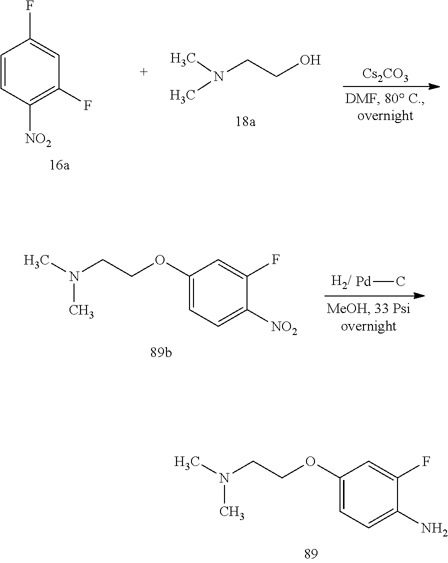

Step 1: Synthesis of 89b: (Part in WO2009076140) The procedure to make 85a was used to isolate 89b as a solid (6.3 g, 44% yield). MS (ESI) 229.1 m/z [M+H]⁺.

Step 2: Synthesis of 89: (Part in WO2002010146) Standard hydrogenation of 89b was followed. The reaction mixture was filtered through a pad of Celite and the filtrate was concentrated under reduced pressure to afford 89 as a solid (5.1 g, 94% yield). MS (ESI) m/z 199.1 [M+H]⁺. ¹H NMR (600 MHz, Chloroform-d) δ 6.62 (dd, J=8.5, 5.7 Hz, 1H), 6.57 (dd, J=10.2, 2.7 Hz, 1H), 6.51 (td, J=8.5, 2.7 Hz, 1H), 4.07 (t, J=5.7 Hz, 2H), 2.76 (t, J=5.7 Hz, 2H), 2.35 (s, 6H).

### Synthesis of 4-(6-methyl-2,6-diazaspiro[3,3]heptan-2-yl)amine (90)

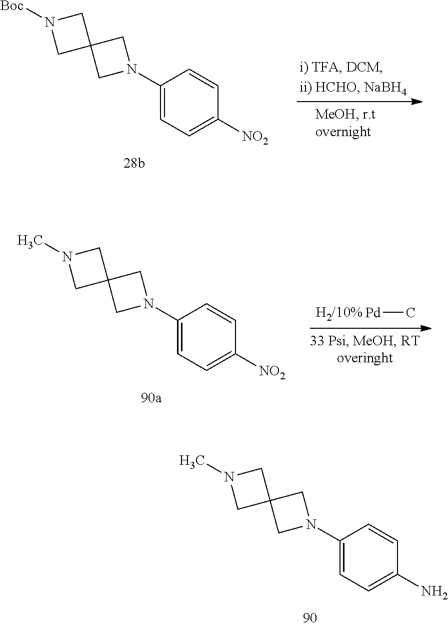

Step 2: Synthesis of 90a: To compound 28b (1.5 g, 5 mmol) was added 25% TFA in DCM (100 mL) and stirred at room temperature for 1 h. After completion of the reaction, removed solvent under vacuum and used as such for the next step. The crude reaction mixture and formaldehyde (2 mL, 25 mmol, 37% aqueous solution) were added to methanol (30 mL), stirred at room temperature for 1 h, then sodium borohydride (946 mg, 25 mmol) was added to the reaction mixture in portions and reaction was continued for overnight. After the reaction is completed, removed solvents under vacuum and the crude residue was purified by silica-gel column using 0-20% MeOH in DCM to give 90a as liquid (701 mg, 60% yield). MS (ESI) 234.1 m/z Step 3: Synthesis of 90: (WO2002010146) To a solution of compound 97c (0.7 g, 3 mmol) in methanol (100 mL) was added catalytic amount of 10% Pd/C-50% wet (0.4 g). The mixture was hydrogenated on a par apparatus at 40 psi for overnight. The reaction mixture was filtered through a pad of Celite and the filtrate was concentrated under reduced pressure to afford 90 as a solid (560 mg, 93% yield). MS (ESI) m/z 204.1 ¹H NMR (600 MHz, Chloroform-d) δ 6.62 (d, J=8.6 Hz, 2H), 6.35 (d, J=8.6 Hz, 2H), 3.84 (s, 3H), 3.39 (s, 4H), 2.33 (s, 4H).

### Synthesis of 3-fluoro-4-(6-methyl-2,6-diazaspiro[3,3]heptan-2-yl)amine (91)

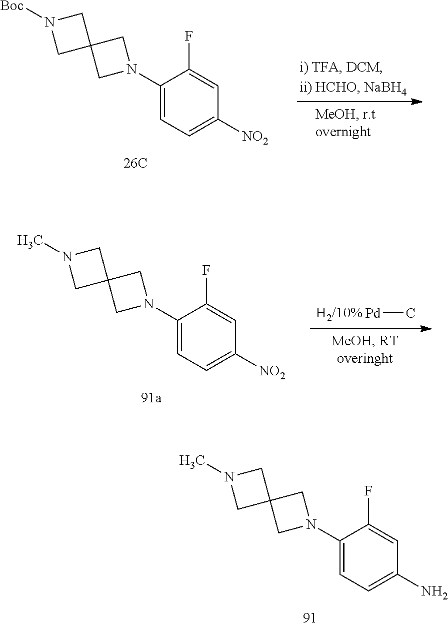

Step 2: Synthesis of 91a: The procedure described for making 90a was used to isolate 91 (601 mg, 48% yield). MS (ESI) m/z 252.1 (VP-HB5-227A) Synthesis of 3-fluoro-4-(6-isopropyl-2,6-diazaspiro[3,3]heptan-2-yl)amine (92):

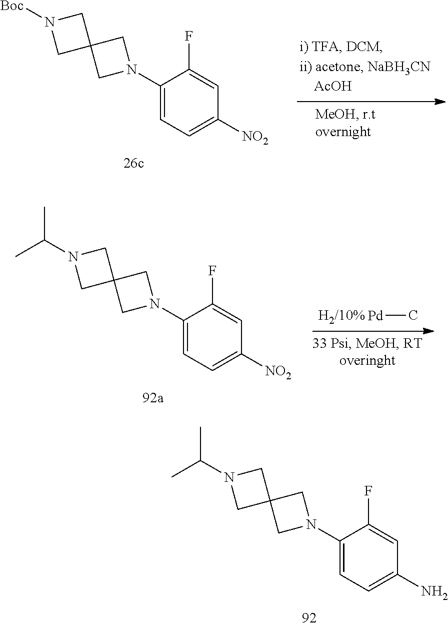

Step 1: Synthesis of 92a: To compound 26c (1.7 g, 5.1 mmol) was added 25% TFA in DCM (100 mL) and stirred at room temperature for 1 h. After completion of the reaction, removed solvent under vacuum and used as such for the next step. The crude reaction mixture, acetic acid (2.5 mL) and acetone (2.5 mL, 51 mmol, 37% aqueous solution) were added to methanol (30 mL), stirred at room temperature for 1 h, then sodium cyanoborohydride (3.2 g, 51 mmol) was added to the reaction mixture in portions and reaction was continued for overnight. After the reaction is completed, removed solvents under vacuum and the crude residue was purified by silica-gel column using 0-20% MeOH in DCM to give 92a as liquid (0.85 g, 61% yield). MS (ESI) m/z 280.1

Step 2: Synthesis of 92: (WO2002010146) To a solution of compound 92a (0.85 g, 3 mmol) in methanol (100 mL) was added catalytic amount of 10% Pd/C-50% wet (0.5 g). The mixture was hydrogenated on a par apparatus at 40 psi for overnight. The reaction mixture was filtered through a pad of Celite and the filtrate was concentrated under reduced pressure to afford 92 as a solid (0.71 g, 93% yield). MS (ESI) m/z 250.2 [M+H]⁺. ¹H NMR (600 MHz, Chloroform-d) δ 6.42-6.30 (m, 3H), 3.90 (s, 4H), 3.33 (s, 4H), 0.93 (d, J=6.2 Hz, 6H).

### Synthesis of 4-(6-isopropyl-2,6-diazaspiro[3.3]heptan-2-yl)amine (93)

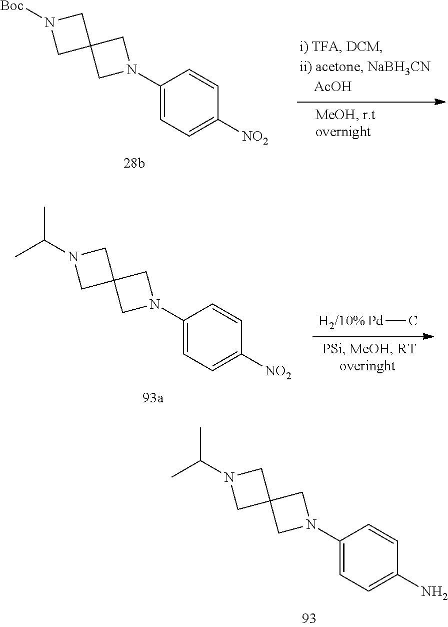

Step 1: Synthesis of 93a: To compound 28b (1.5 g, 5 mmol) was added 25% TFA in DCM (100 mL) and stirred at room temperature for 1 h. After completion of the reaction, removed solvent under vacuum and used as such for the next step. The crude reaction mixture, acetic acid (2.5 mL) and acetone (2.5 mL, 50 mmol, 37% aqueous solution) were added to methanol (30 mL), stirred at room temperature for 1 h, then sodium cyanoborohydride (3.1 g, 50 mmol) was added to the reaction mixture in portions and reaction was continued for overnight. After the reaction is completed, removed solvents under vacuum and the crude residue was purified by silica-gel column using 0-20% MeOH in DCM to give 93a as liquid (0.86 g, 66% yield). MS (ESI) m/z 262.1

Step 2: Synthesis of 93: (WO2002010146) To a solution of compound 93a (0.86 g, 3.3 mmol) in methanol (100 mL) was added catalytic amount of 10% Pd/C-50% wet (0.5 g). The mixture was hydrogenated on a par apparatus at 40 psi for overnight. The reaction mixture was filtered through a pad of Celite and the filtrate was concentrated under reduced pressure to afford 93 as a solid (0.74 g, 97% yield). MS (ESI) m/z 232.2 [M+H]⁺. ¹H NMR (600 MHz, Chloroform-d) δ 6.63 (d, J=8.6 Hz, 2H), 6.35 (d, J=8.6 Hz, 2H), 3.84 (s, 4H), 3.32 (s, 4H), 2.24 (dt, J=12.4, 6.2 Hz, 1H), 0.93 (d, J=6.2 Hz, 6H).

### Synthesis of 4-(4-isopropylpiperidin-1yl)aniline (94)

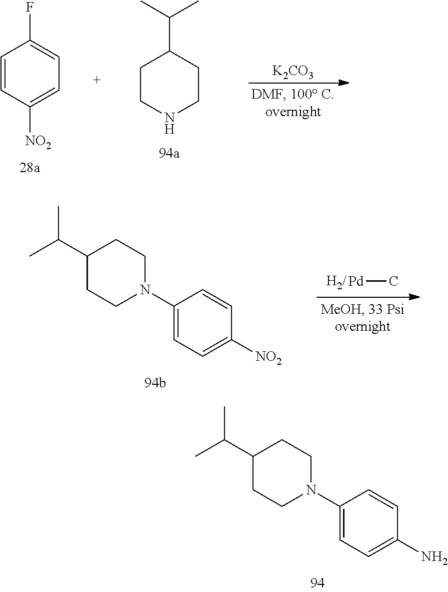

Step 1: Synthesis of 94: (Part in WO2009079597) To a stirred mixture of compound 28a (1.1 g, 8 mmol) in DMF (25 mL) at room temperature were added 101a (1.0 g, 8 mmol) and K₂CO₃ (3.3 g, 24 mmol). The resulting mixture was stirred at 80° C. for overnight. Reaction mixture was cooled and diluted with water (200 mL) and the solid obtained was filtered and washed with water (3×25 mL) and dried under vacuum to give compound 94a as a yellow solid (1.7 g, 87% yield). MS (ESI) m/z 249.1 [M+H]⁺. This product is used as such in the next step.

Step 2: Synthesis of 94: (WO2002010146) The standard hydrogenation technique and isolation procedure afforded 94 as a solid (1.3 g, 87% yield). MS (ESI) m/z 219.2 [M+H]⁺. ¹H NMR (600 MHz, Chloroform-d) δ 6.83 (d, J=8.7 Hz, 2H), 6.64 (d, J=8.7 Hz, 2H), 3.52-3.44 (m, 2H), 2.58-2.46 (m, 2H), 1.80-1.70 (m, 2H), 1.54-1.37 (m, 3H), 1.09 (dddd, J=15.5, 11.9, 6.8, 3.7 Hz, 1H), 0.91 (d, J=6.8 Hz, 6H).

The Following Amines were Purchased from Commercial Sources:

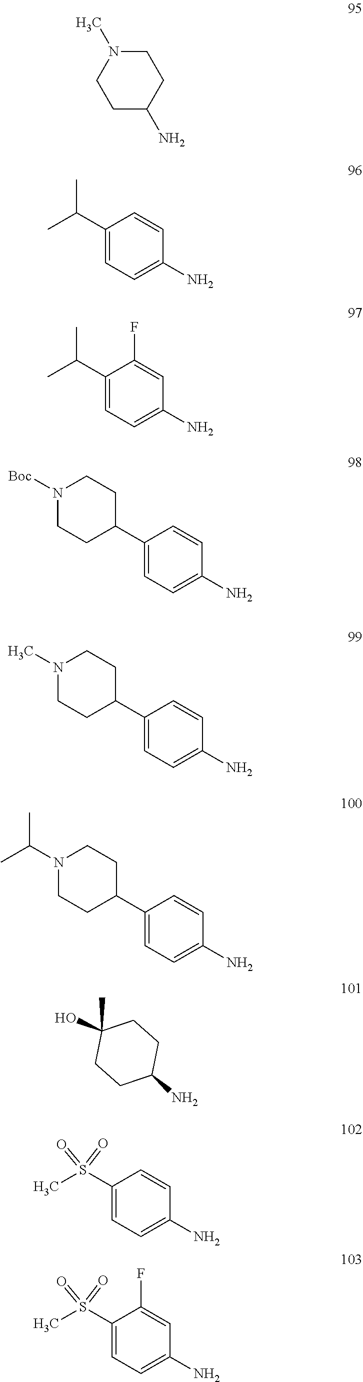

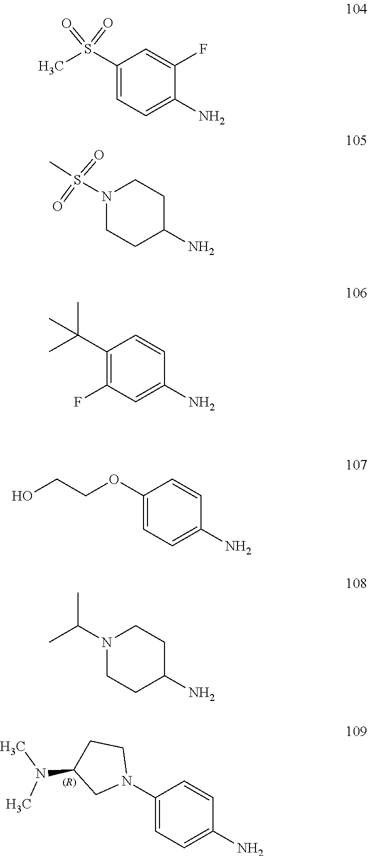

Synthesis of 109: Prepared according to the procedure described for the preparation of compound 33. MS (ESI) m/z 206.2 [M+H]⁺.

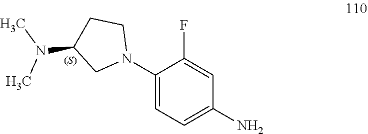

Synthesis of 110: Prepared according to the procedure described for the preparation of compound 30. MS (ESI) m/z 224.2 [M+H]⁺.

Some of the amines that are not mentioned here are commercially available and might have used to make the final target molecules.

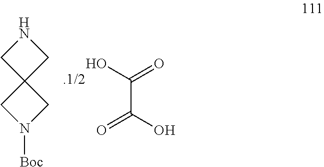 [SMILES: CC(C)(C)OC(=O)N1CC2(CNC2)C1 ; O=C(O)C(=O)O]

Procured from a commercial source.

### Example 2

### General Synthesis and Schemes

General Methods. General reaction conditions are described in International Pat. App. Publication No. WO 2018009625, which is incorporated by reference for such teachings. The reactions were generally carried out under a dry argon atmosphere using anhydrous solvents (Sigma-Aldrich, St. Louis, Mo.). All common chemicals were purchased from commercial sources. The purification of the molecules was carried out using Combi Flash Rf from Teledyne ISCO, regular silica gel columns, and C18 columns. Chiral HPLC columns (Chiral Technologies) and lyophilization techniques (VirTis Genesis 25L Pilot Lyophilizer) were used wherever necessary to isolate the desired products. The isolated target products are excellent powder/fluffy material and possess the purity, in general, of >97%. ¹H NMR spectra were recorded on a 600 MHz Fourier Transform Brücker Spectrometer. Spectra were obtained from samples prepared in 5 mm diameter tubes in CDC₃, CD₃OD or DMSO-d₆. The spin multiplicities are indicated by the symbols, s (singlet), d (doublet), t (Triplet), m (multiplet) and, br (broad). Coupling constants (J) are reported in Hz. MS spectra were obtained using electrospray ionization (ESI) on an Agilent Technologies' 6120 quadrupole LCMS system.

The compounds described herein may be synthesized by many methods available to those skilled in the art of organic chemistry. Maffrand et al., Heterocycles 16(1):35-37 (1981). General synthetic schemes for preparing compounds described herein are described below. These schemes are illustrative and are not meant to limit the possible techniques one skilled in the art may use to prepare the compounds disclosed herein. Different methods to prepare the compounds described herein will be evident to those skilled in the art. Additionally, the various steps in the synthesis may be performed in an alternate sequence in order to give the desired compound or compounds. Examples of compounds described herein prepared by methods described in the general schemes are shown below.

Preparation of homochiral examples may be carried out by techniques known to one having ordinary skill in the art. For example, homochiral compounds may be prepared by separation of racemic products by chiral phase preparative HPLC. Alternatively, the example compounds may be prepared by methods known to give enantiomerically enriched products such as asymmetric synthesis and enantioselective synthesis. These include, but are not limited to, the incorporation of chiral auxiliary functionalities into racemic intermediates that serve to control the diastereoselective of transformations, providing enantio-enriched products upon cleavage of the chiral auxiliary. The compounds described herein can be prepared in a number of ways known to one skilled in the art of organic synthesis. The compounds described herein can be synthesized using the methods described below, together with synthetic methods known in the art of synthetic organic chemistry, or by variations thereon as appreciated by those skilled in the art. Preferred methods include, but are not limited to, those described below. The reactions are performed in a solvent or solvent mixture appropriate to the reagents and materials employed and suitable for the transformations being affected. It will be understood by those skilled in the art of organic synthesis that the functionality present on the molecule should be consistent with the transformations proposed. This will sometimes require a judgment to modify the order of the synthetic steps or to select one process scheme over another in order to obtain a desired compound described herein. It will also be recognized that another major consideration in the planning of any synthetic route in this field is the judicious choice of the protecting group used for protection of the reactive functional groups present in the compounds described herein. An authoritative account describing the many alternatives to the trained practitioner is Greene et al., Protective Groups in Organic Synthesis, 4^{th} ed, Wiley-Interscience (2006).

### General Scheme for Compounds

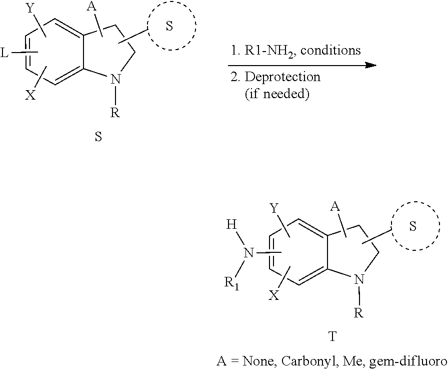

Scheme T can be followed to make the final target molecules. The synthesis of variously substituted starting materials (S) can be made using one or more of the schemes shown below. It may be modified the proposed synthetic plan to affect the desired transformation. R₁NH₂ can be obtained from the commercial source, or it can be made using the standard reactions (Scheme A) that are appreciated by those skilled in the art. Group L may be changed to L1 (e.g., SMe to SOMe/SO₂Me) before reacting with R₁NH₂ in the sequence.

General Scheme for Making R₁NH₂

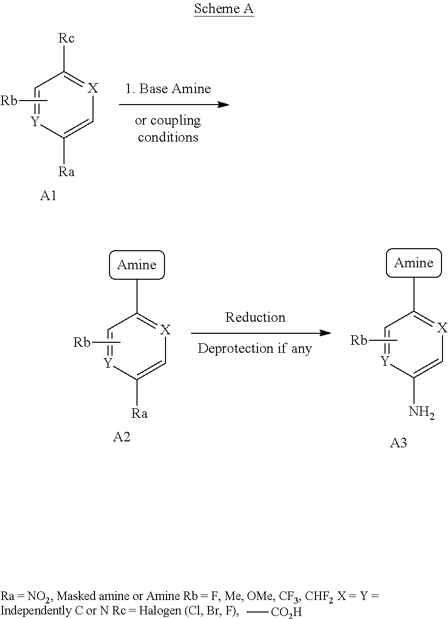

The R₁NH₂ required for making the final targets would be synthesized via a sequence shown in the scheme A. The appropriately substituted A1 can give the intermediate or the final amine A2 via a reaction(s) such as displacement of halogen atom or amide bond formation reactions that are appreciated by those skilled in the art. The desired A3 (R₁NH₂) can be obtained from A2 by reduction of a nitro group or de-masking the amine as needed.

### General Schemes to Make Building Blocks and Final Target Molecules

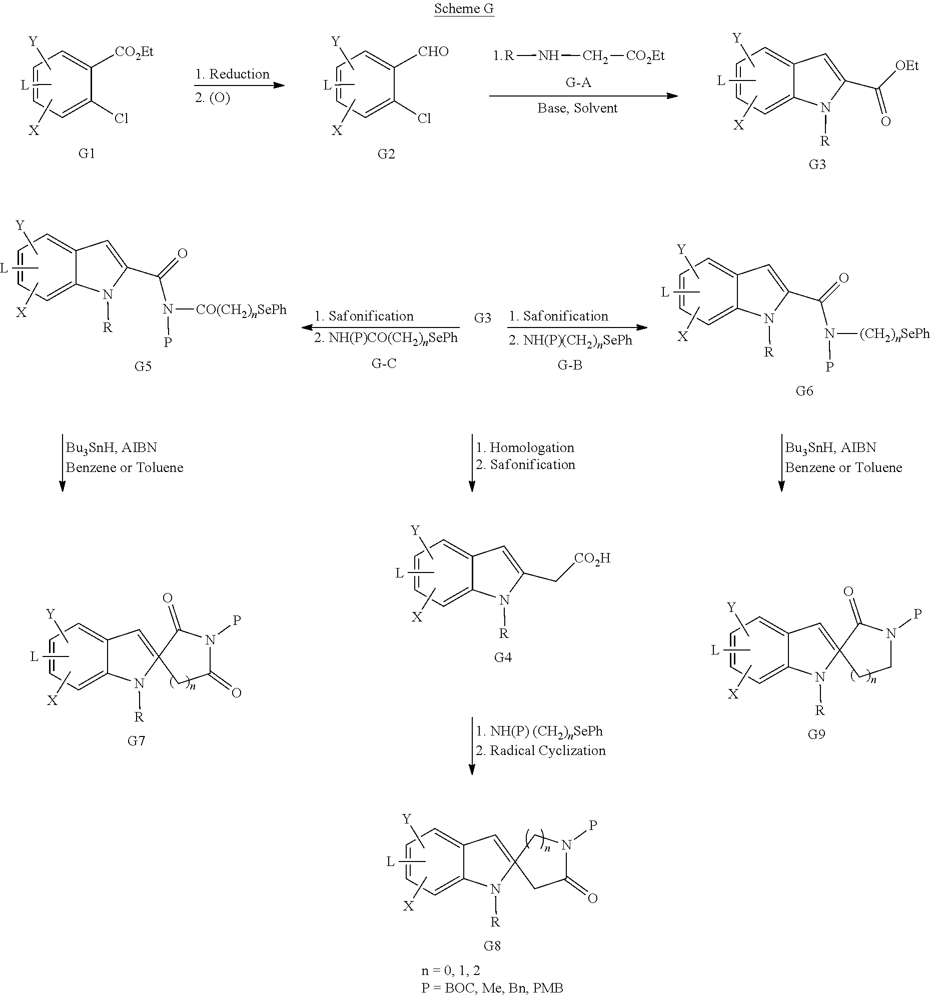

As shown in Scheme G, compounds depicted in the general claim may be synthesized from a readily available starting material. The required intermediates and the target molecules can be made using the standard synthetic organic chemistry procedures. The reported standard reaction conditions on a substrate can be extended to perform the reactions on analogs substrates to effect the transformation. The bicyclic compound G3 can be obtained by reaction between G2 and a counter partner G-A under necessary conditions. See e.g., Zhu et al., Eur. J. Org. Chem. 2014(3): 511-514 (2014). The ester G3 upon saponification and coupling with the required selenide moiety G-B (prepared from the appropriately substituted halide using the known procedure in the art) gave the substrate G6, which upon a radical cyclization (see e.g., Kyei et al., Tetrahedron Lett. 45(48): 8931-8934 (2004)) to afford the tricyclic spiro compound G9. Likewise, the compound G7 can be prepared via an intermediacy of G5. The compound G3 can be transformed into higher homolog G4 via Arndt-Eistert Synthesis. The tricyclic compound G8 can be obtained by a two-step reaction starting from G4, coupling with selenide G-B followed by radical cyclization. The tricyclic compounds G7, G8, and G9 will serve as penultimate compounds to synthesize the final target molecules.

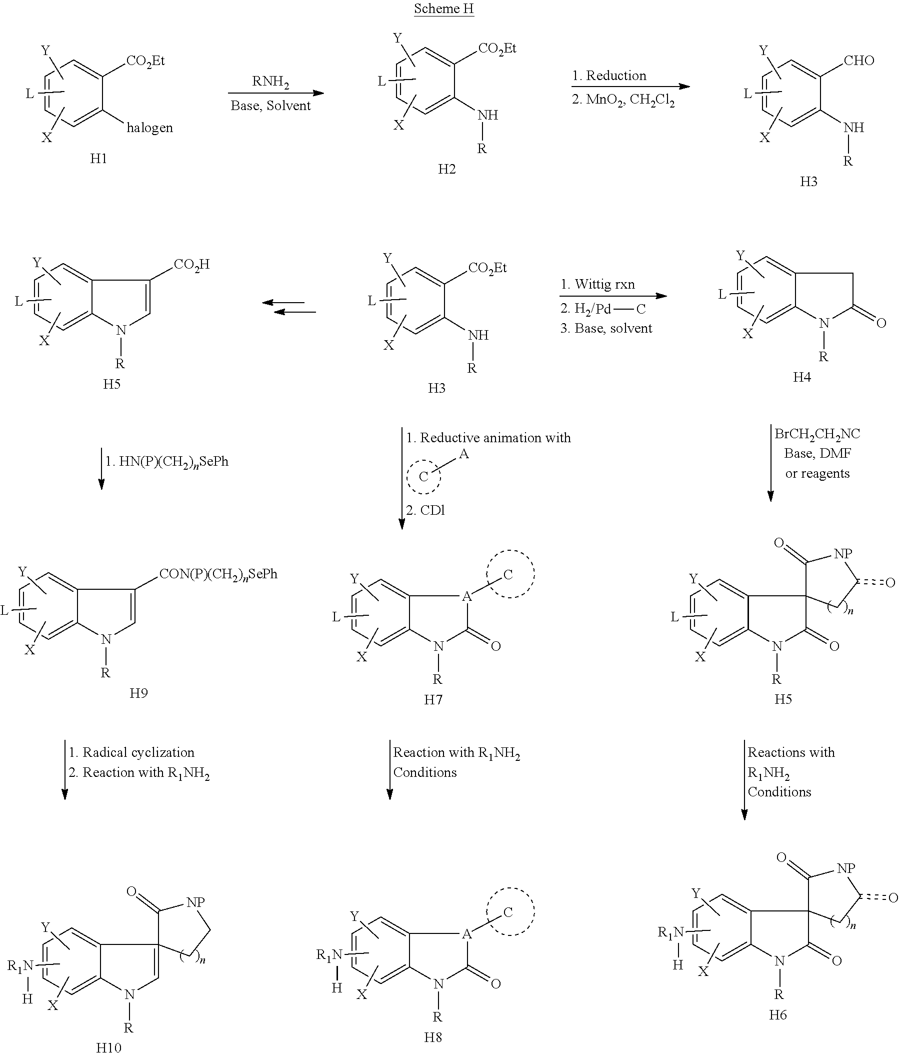

Scheme H gives the detailed synthetic avenues to make key building blocks as well as the target molecules. Compound H1 can be transformed to the key intermediate H3 following the standard reactions. The bicyclic compound H4 can be synthesized starting from an aldehyde H3 or an appropriately substituted compound (see WO 2001055148). The compound type H4 can also be made by following alternative literature reports. See e.g., WO 2005047289; WO 2016171755; Putey et al., J. Med. Chem. 52(19): 5916-5925 (2009). The compound H4 upon a reaction with appropriate isocyanate in a single step or using a sequence of reactions with appropriate reagents gives the tricyclic spirocompound of the type H5. See e.g., WO 2006125784; Makkay et al., Tetrahedron 56 (32): 5893-5903 (2000); Crosignani et al., J. Med. Chem. 51(7): 2227-2243(2008). Compound H7 can be achieved from H3 employing reductive amination with the appropriate amine followed by a reaction with carbonyl diimidazole or oxalyl chloride. See WO 2014144737; WO 2000024744. Compound H3 can also serve as a starting material to obtain the acid H5. See WO 2005047289; WO 2016171755; Putey et al., J. Med. Chem. 52(19): 5916-5925 (2009). The compound H5 upon coupling with a selenide moiety provided the intermediate H9 and subsequent radical cyclization of it gives the the penultimate spiro substrate to react with R₁NH₂ to afford the final targets of the type H10. The proposed routes or slightly modified schemes may work to afford the desired target molecules and below are some illustrated schemes.

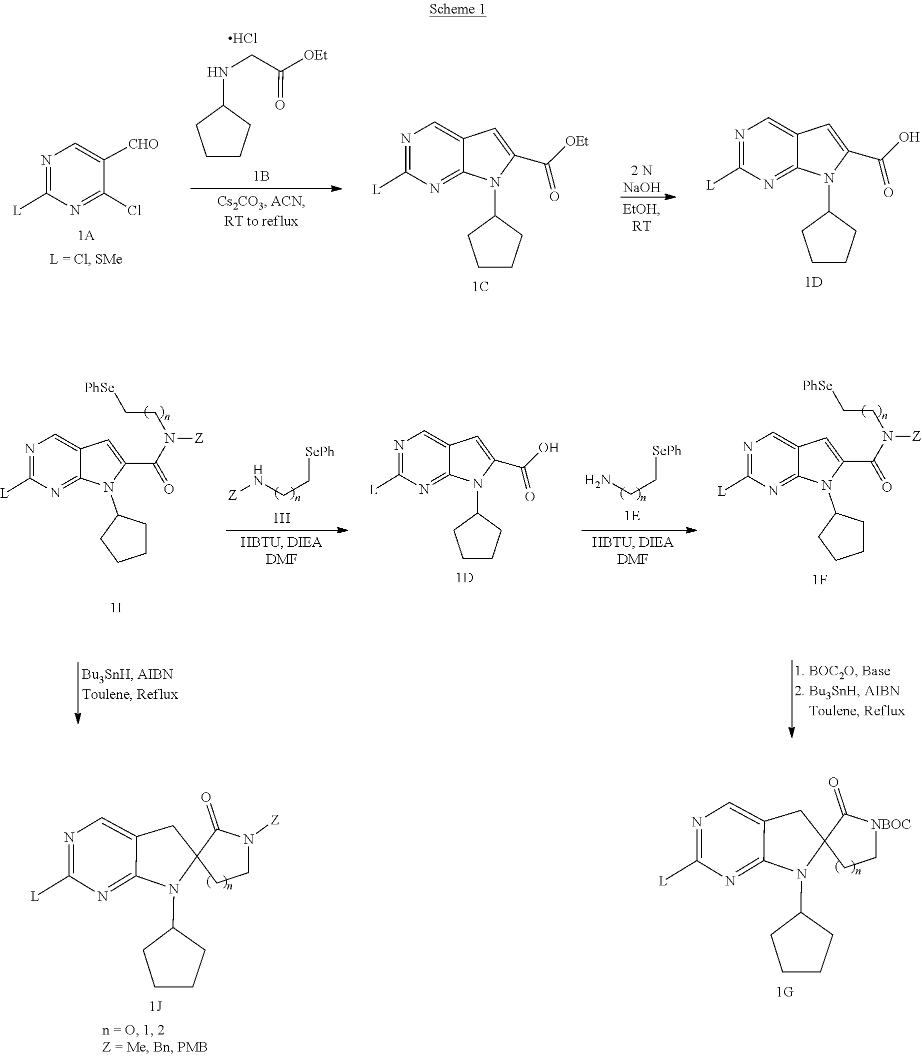

Scheme I provides synthetic routes of making the tricyclic 2-spiro compounds. The aldehyde 1A was converted to bicyclic compound 1C via Knoevenagel condensation with amino ester 1B (synthesized from a reaction between cyclopentyl amine and bromoethylacetate) followed by cyclization. See e.g., Zhu et al., Eur. J. Org. Chem. 2014(3): 511-514 (2014). The acid 1 D can be coupled with selenide 1E under standard conditions gives an advanced intermediate 1F. The compound 1F was protected with BOC and then subjected for radical cyclization to afford 1G. A similar sequence was used to make tricyclic 1J via an intermediacy of 11. The scheme can be used to make the penultimate variously tricyclic 2-spiro compounds 1J and 1G.

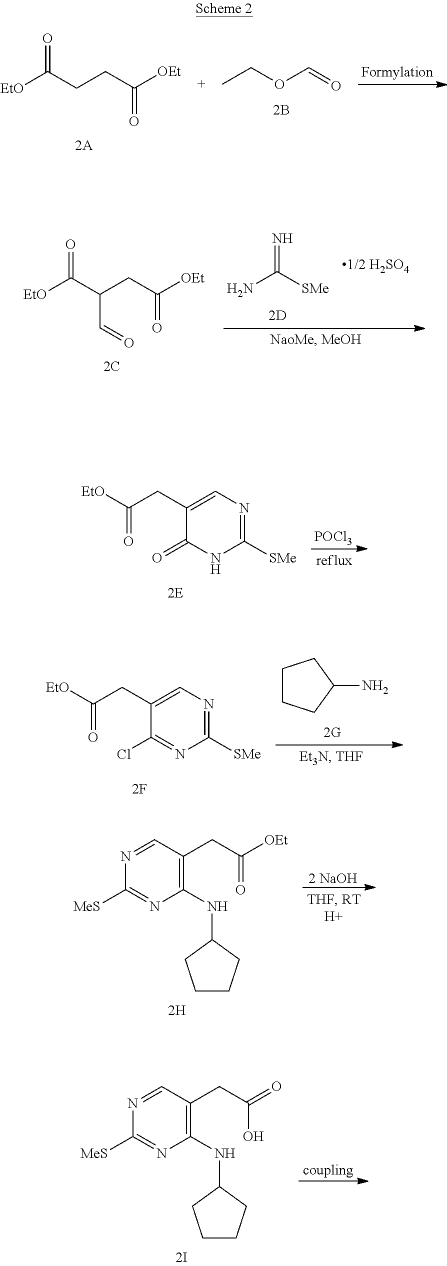

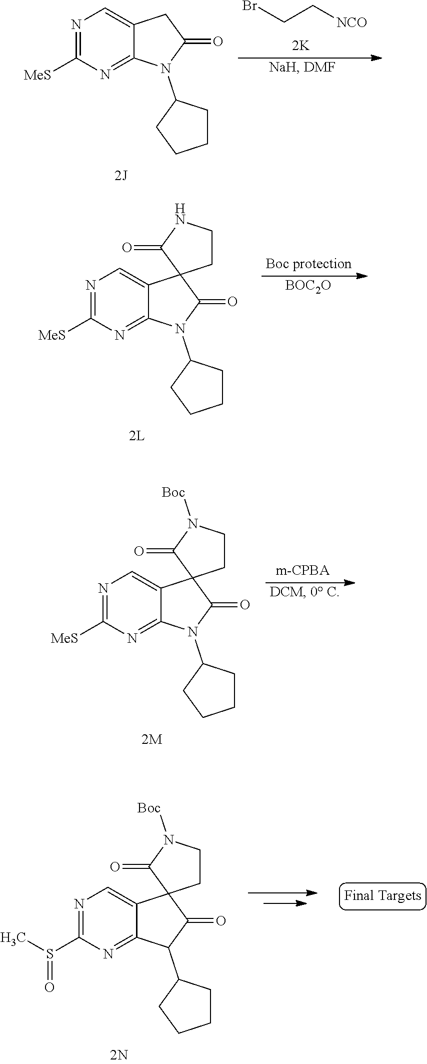

Scheme 2 provides synthetic routes of making the tricyclic 3-spiro compound 2N. The key compound 2J can be made by following the methods known in the art. See WO 2009152027. The analogous procedure reported for making 2M type compounds was followed. See Crosignani et al., J. Med. Chem. 51(7): 2227-2243 (2008).

The following schemes show enantioselective synthesis of spirocyclic compounds:

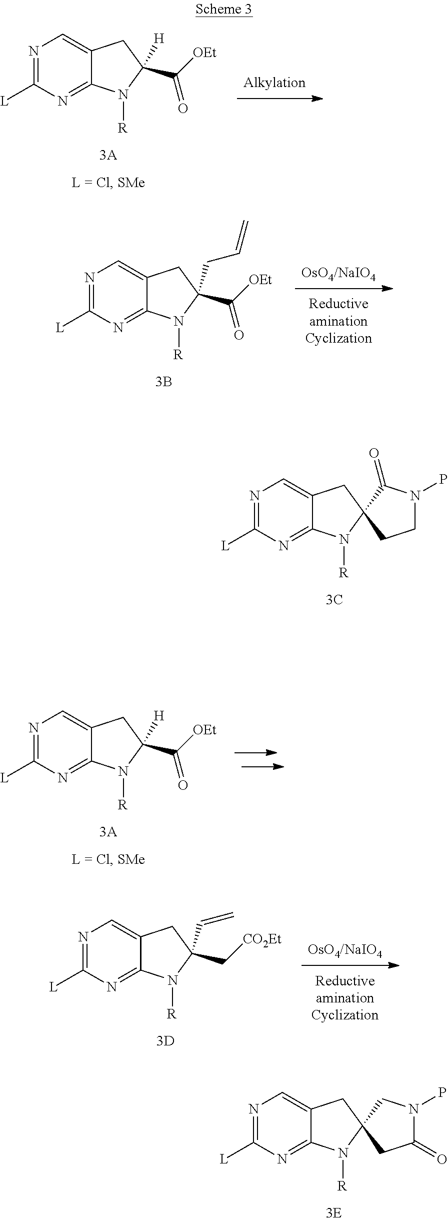

Scheme 3 describes the synthesis of enantiopure 2-spiro compounds. The sequence of steps shown in this scheme is known in the art. The choice of regents will dictate the outcome of the reaction (racemic or enantiopure). Many of the transformations and the general reagents needed to affect the transformation will be disclosed in the general Scheme G along with the associated literature reports.

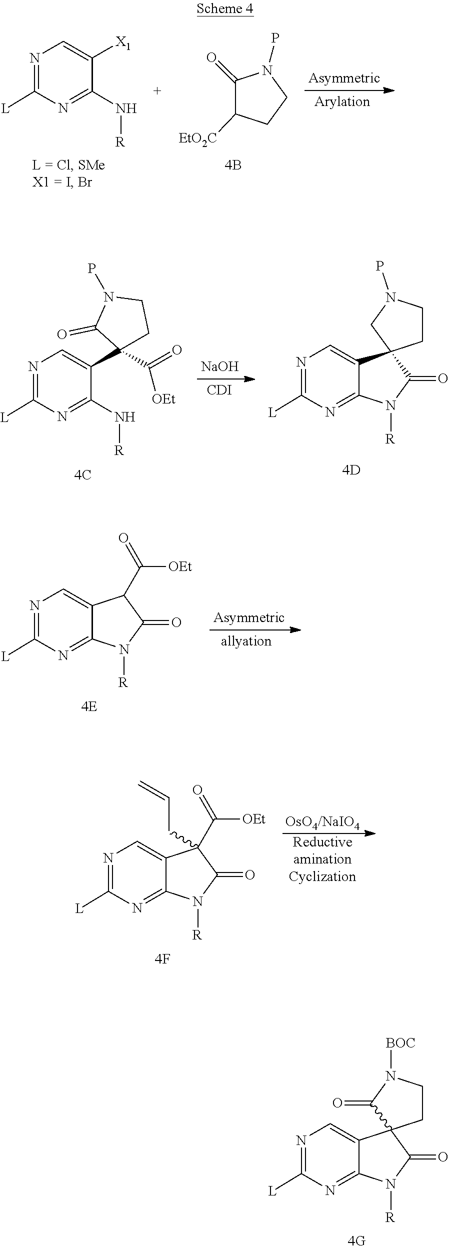

Scheme 4 describes the synthesis of 3-spiro tricyclic compounds that serve as starting material to make the final targets in the enantiopure molecules. Many reports describe the synthesis of spiro compounds, for example: Benabdallah et al., Curr. Med. Chem. 25(31): 3748-3767 (2018); Rios, Chem. Soc. Rev. Rev. 41(3): 1060-1074 (2012); Ding et al., Chem. Soc. Rev. 47 (15): 5946-5996 (2018); Singh et al., Beilstein J. Org. Chem. 14: 1778-1805 (2018).

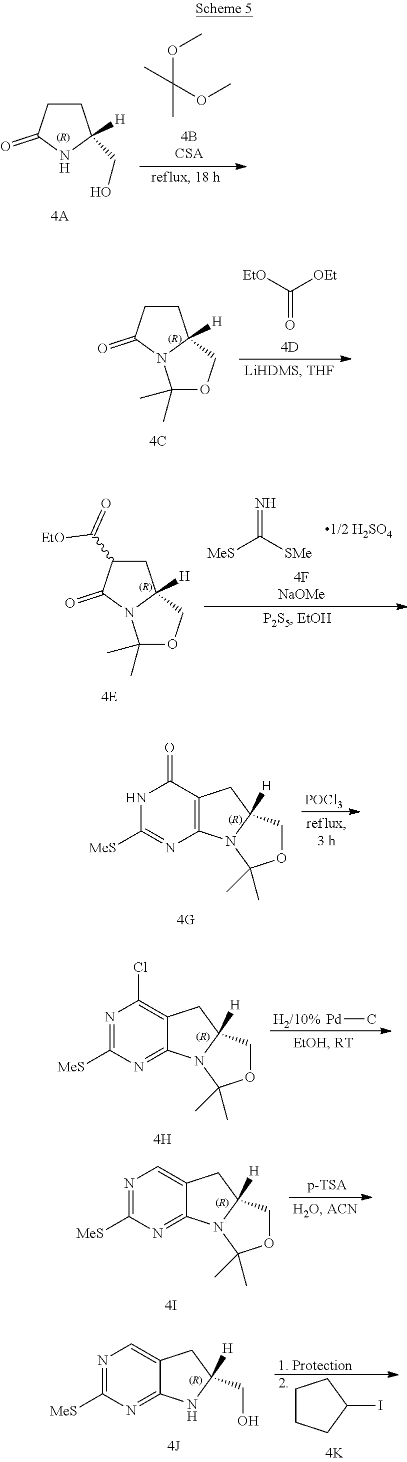

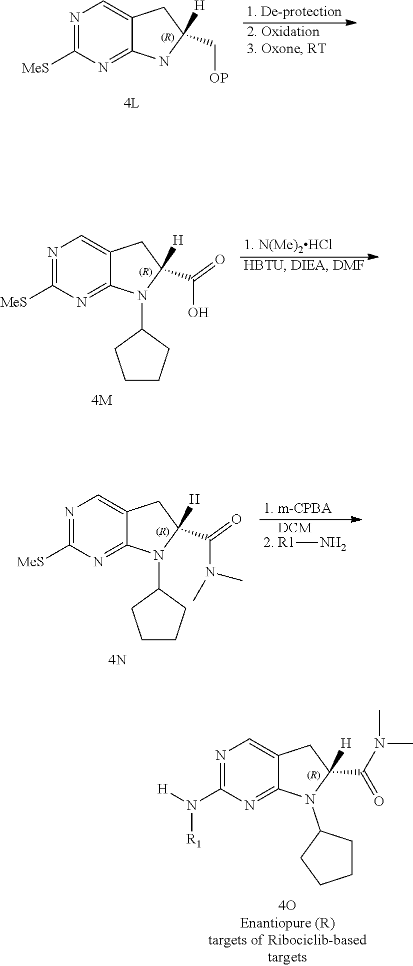

### Synthesis of Enantiopure Ribociclib-Based Targets

Scheme 5 shows the steps that can be used to synthesize enantiopure key intermediates and subsequently the final target molecules of the type 40. The synthesis starts with an enantiopure commercially available (R)-5-(hydroxymethyl)-5-methylpyrrolidin-2-one, 4A. A diastereomeric mixture 4E can be obtained from the compound 4C. The compound 4E can be converted to an advanced intermediate 4G by following the known reaction conditions in the art. Conversition of 4G to 41 can be achieved via an intermediacy of 4H. The compound 41 upon treatment with p-TSA gives the alcohol 4J. The compounds of the type 4J can also be obtained from a different sequence. N-alkylation with cyclopentyl iodide upon protecting the alcohol can give the compound 4L. The de-protection of 4L followed by oxidation of the resulting alcohol gives the corresponding aldehyde. The oxidation of an intermediate aldehyde with oxone provides the carboxylic acid 4M. The desired amide 4N can be obtained using dimethylamine and coupling reagents. The compound 4N can be used to make the final targets of the type 40 via an intermediacy sulfoxide of 4N. Many of the steps are straightforward in that one can make enantiopure targets following the sequence. Shown are the scheme details to make R-enantiomer, and the same sequence can be employed to make S-enantiomer of 40. It can be accomplished by starting with (S)-5-(hydroxymethyl)-5-methylpyrrolidin-2-one. See WO 2009107767 WO 2012101013; WO 2015009991; WO 2015129926; Davies, et al., Tetrahedron: Asymmetry 13(6): 647-658 (2002); Moss et al., Synlett. 23(16): 2408-2412 (2012); Chavda et al., Eur. J. Org. Chem. 2014(1):129-139 (2014).

### Synthesis of Enantiopure Trilaciclib-based Targets

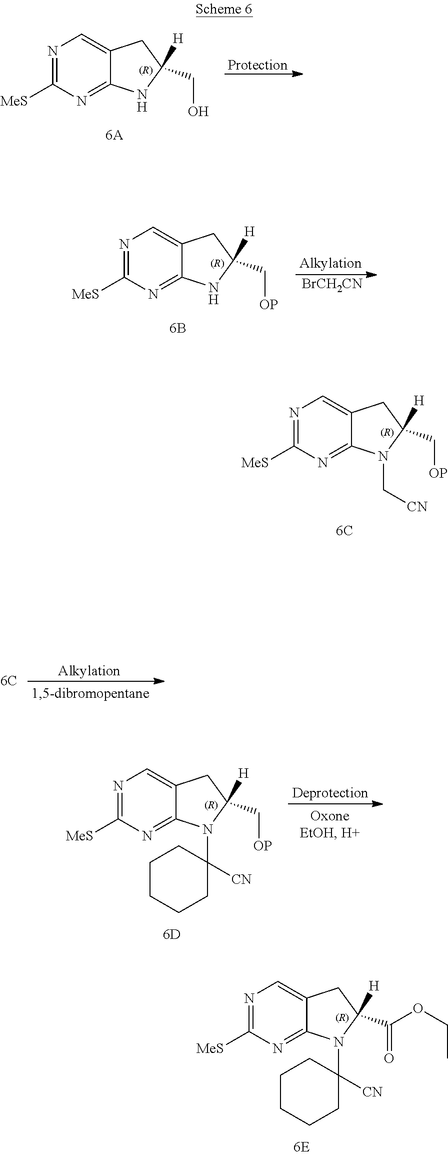

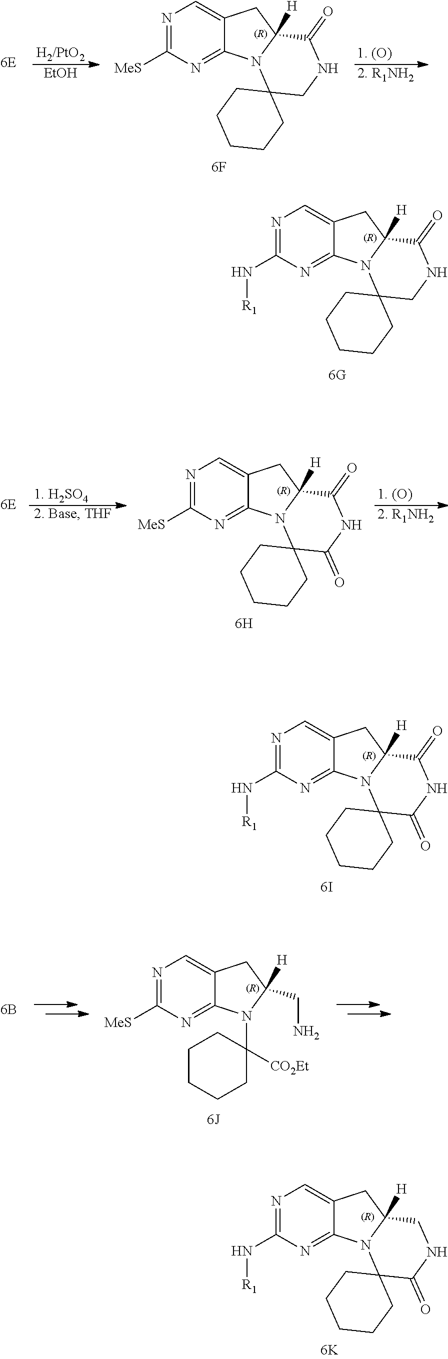

The steps shown in Scheme 6 show the synthesis of enantiopure tetracyclic spiro compound 6J and the target molecule of the type 6K. The sequence of steps described in Scheme 7 can affords enantiomeric pure targets of type 6K. Scheme 6 will produce both R- and S-enantiomers of the target molecules depending on the starting material. The compound R-isomer 6A can give the R-eantiomer of the type 6G, 6I, and 6K. The corresponding S-enantiomers of the type 6G, 6I, and 6K can also be obtained from the S-enantiomer of 6A. Related chemistry of achiral substrtates can be extended to use on the enantiopure substrates. See WO 2018005860.

### Example 3

### General Procedure for the Synthesis of Compounds C1-C107

### General Procedure A

Parts of the procedure were described in WO 2010020675, which is incorporated by reference herein for such teachings. A mixture of corresponding amine (1.5 eq), amide (1 eq), BINAP (20 mole percent) and sodium-tert-butoxide (2 eq) in dioxane was degassed and Pd₂(dba)₃ (10 mole percent) was added under argon. The reaction mixture was heated to 100° C. for 2 h in an oil bath. After completion, the reaction mixture was quenched with MeOH, concentrated and purified using silica gel chromatography (0 to 100% MeOH in dichloromethane). The obtained residue was dissolved and stirred in 5 mL of 30% TFA/DCM solution for 2 h at RT. The mixture was concentrated to dryness and treated with 7 N NH₃/MeOH solution. The mixture was concentrated again and purified using a C18 column (0 to 100% CH₃CN in H₂O with 0.025% AcOH). The fractions containing desired compound were combined and lyophilized.

### General Procedure B

Parts of the procedure were described in WO 2010020675, which is incorporated by reference herein for such teachings. A mixture of corresponding amine (1.5 eq), amide (1 eq), BINAP (20 mole percent) and sodium-tert-butoxide (2 eq) in dioxane was degassed and Pd₂(dba)₃ (10 mole percent) was added under argon. The reaction mixture was heated to 100° C. for 2 h in an oil bath. After completion, the reaction mixture was quenched with MeOH, concentrated, and purified using silica gel chromatography (0 to 100% MeOH in dichloromethane). The fractions containing required compound were collected, concentrated and further purified on a C18 column (0 to 100% ACN in H₂O) to give the desired compound. The fractions containing desired compound were combined and lyophilized.

### General Procedure C

Parts of the procedure were described in U.S. Pat. No. 7,776,869, which is incorporated by reference herein for such teachings. A mixture of amide (1 eq), corresponding amine (1.5 eq), TFA (10 eq.) in 2-propanol were heated at 100° C. in a sealed tube for 24 h. After completion, the reaction mixture was concentrated and purified using silica gel chromatography (0 to 100% Methanol in dichloromethane). The fractions containing required compound were collected and concentrated under reduced pressure. The obtained residue was dissolved and stirred in 5 mL of 30% TFA/DCM solution for 2 h at RT. The mixture was concentrated to dryness and treated with 7 N NH₃/MeOH solution. The mixture was concentrated again and purified using a C18 column (0 to 100% CH₃CN in H₂O with 0.025% AcOH). The fractions containing desired compound were combined and lyophilized.

### General Procedure D

Parts of the procedure were described in Chinese Patent Application No. CN 106749259, published as CN 201510800951, both of which are incorporated by reference herein for such teachings. To a solution of corresponding amine (3 eq) in anhydrous toluene was added 1 M LiHMDS in toluene (3.6 eq) dropwise at RT. After stirring for 30 minutes, a solution of amide (1 eq) in anhydrous toluene was added dropwise and stirring was continued for 1 h. The reaction mixture is then quenched with AcOH, concentrated and purified using silica gel chromatography (0 to 100% methanol in dichloromethane). The fractions containing required compound were collected and concentrated under reduced pressure. The obtained residue was dissolved and stirred in 5 mL of 30% TFA/DCM solution for 2 h at RT. The mixture was concentrated to dryness and treated with 7 N NH₃/MeOH solution. The mixture was concentrated again and purified using a C18 column (0 to 100% CH₃CN in H₂O with 0.025% AcOH). The fractions containing desired compound were combined and lyophilized.

### General Procedure E

Parts of the procedure were described in WO 2018005865, which is incorporated by reference herein for such teachings. To a stirred solution of corresponding amine (3 eq) in anhydrous THF was added LiHMDS (1.2 eq) dropwise at 0° C. After stirring for 15 minutes at RT, corresponding amide (1 eq) in anhydrous THF was added dropwise at RT and stirred for 3 h. After completion, the reaction mixture was quenched with AcOH, concentrated and purified using silica gel chromatography (0 to 100% methanol in dichloromethane). The fractions containing required compound were collected and concentrated under reduced pressure. The obtained residue was dissolved and stirred in 5 mL of 30% TFA/DCM solution for 2 h at RT. The mixture was concentrated to dryness and treated with 7 N NH₃/MeOH solution. The mixture was concentrated again and purified using a C18 column (0 to 100% CH₃CN in H₂O with 0.025% AcOH). The fractions containing desired compound were combined and lyophilized.

### General Procedure F

A mixture of amide (1 eq), corresponding amine (1 eq) in toluene was heated at reflux for 24 h. After completion, the reaction mixture was concentrated and purified using silica gel chromatography (0 to 100% methanol in dichloromethane). The fractions containing required compound were collected and concentrated under reduced pressure. The obtained residue was dissolved and stirred in 5 mL of 30% TFA/DCM solution for 2 h at RT. The mixture was concentrated to dryness and treated with 7 N NH₃/MeOH solution. The mixture was concentrated again and purified using a C18 column (0 to 100% CH₃CN in H₂O with 0.025% AcOH). The fractions containing desired compound were combined and lyophilized.

### General Procedure G

Parts of the procedure were described in WO 2010065721, which is incorporated by reference herein for such teachings. A mixture of amide (1 eq), corresponding amine (1.5 eq), CSA (3 eq) in N, N-Dimethyl acetamide were heated in a RB flask at 140° C. for 1.5 h. After completion, the reaction mixture was concentrated and purified using silica gel chromatography (0 to 100% Methanol in dichloromethane). The fractions containing required compound were collected and concentrated under reduced pressure. The obtained residue was dissolved and stirred in 5 mL of 30% TFA/DCM solution for 2 h at RT. The mixture was concentrated to dryness and treated with 7 N NH₃/MeOH solution. The mixture was concentrated again and purified using a C18 column (0 to 100% CH₃CN in H₂O with 0.025% AcOH). The fractions containing desired compound were combined and lyophilized.

### General Procedure H

Parts of the procedure were described in WO 2010020675, which is incorporated by reference herein for such teachings. A mixture of corresponding amine (1.5 eq), amide (1 eq), BINAP (20 mole percent) and sodium-tert-butoxide (2 eq) in dioxane was degassed and Pd₂(dba)₃ (10 mole percent) was added under argon. The reaction mixture was heated to 100° C. for 2 h in an oil bath. After completion, the reaction mixture was quenched with MeOH, concentrated and purified using silica gel chromatography (0 to 100% methanol in dichloromethane). The fractions containing required compound were collected and concentrated under reduced pressure. The obtained residue was dissolved and stirred in 5 mL of 30% TFA/DCM solution for 2 h at RT. The mixture was concentrated to dryness and treated with 7 N NH₃/MeOH solution. The mixture was concentrated again and purified using a C18 column (0 to 100% CH₃CN in H₂O with 0.025% AcOH). The fractions containing desired compound were combined and lyophilized.

General Procedure I

Parts of the procedure were described in WO 2016105564, which is incorporated by reference herein for such teachings. Amide (1 eq.), corresponding amine (1.5 eq), N, N-diisopropylethylamine (8 eq.), Xantphos (30 mole percent) and palladium (II) acetate (20 mole percent) were combined under argon in NMP. The reaction mixture was heated at 150° C. in a sealed vessel for 4 h. The reaction mixture was then cooled to RT, absorbed on silica gel and purified using silica gel chromatography (0 to 100% methanol/dichloromethane). The fractions containing required compound were collected and concentrated under reduced pressure. The obtained residue was dissolved and stirred in 5 mL of 30% TFA/DCM solution for 2 h at RT. The mixture was concentrated to dryness and treated with 7 N NH₃/MeOH solution. The mixture was concentrated again and purified using a C18 column (0 to 100% CH₃CN in H₂O with 0.025% AcOH). The fractions containing desired compound were combined and lyophilized.

### Example 1: Compound C33

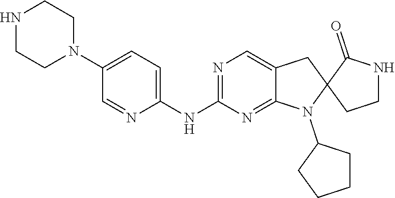 [SMILES: O=C1NCCC12Cc1cnc(Nc3ccc(N4CCNCC4)cn3)nc1N2C1CCCC1]

### Step 1: Synthesis of 2-(phenylselanyl)ethan-1-amine (1-B)

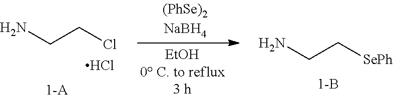

(Ref: Kyei et al., Tetrahedron Lett. 45(48): 8931-8934 (2004)): To a stirred solution of diphenyl selenide (20 g, 65 mmol) in ethanol (200 mL) at 0° C. was added NaBH₄(12 g, 322.5 mmol) portion wise under argon. After stirring for 15 minutes at the same temperature, compound 1-A (15 g, 129 mmol) in ethanol (200 mL) was added dropwise at 0° C. The reaction mixture was stirred at 0° C. for 30 min and at reflux temperature for 3 h. After completion, the reaction mixture was quenched with brine and the solvents were removed under reduced pressure. The crude is partitioned between ethyl acetate (500 mL) and saturated NaHCO₃ (300 mL). After the layers were separated, the organic layer was washed with brine, dried over sodium sulfate, concentrated and purified by column chromatography (0-10% MeOH in DCM) to get compound 1-B (20 g, 78% yield). MS(ESI) m/z 202.1 (M+H)⁺. ¹H NMR (600 MHz, Chloroform-d) δ 7.55-7.50 (m, 2H), 7.26 m, 3H), 3.01 (m, 6H).

Step 2: Synthesis of tert-butyl 2′-chloro-7′-cyclopentyl-2-oxo-5′,7′-dihydrospiro[pyrrolidine-3,6′-pyrrolo[2,3-d]pyrimidine]-1-carboxylate (1-F)

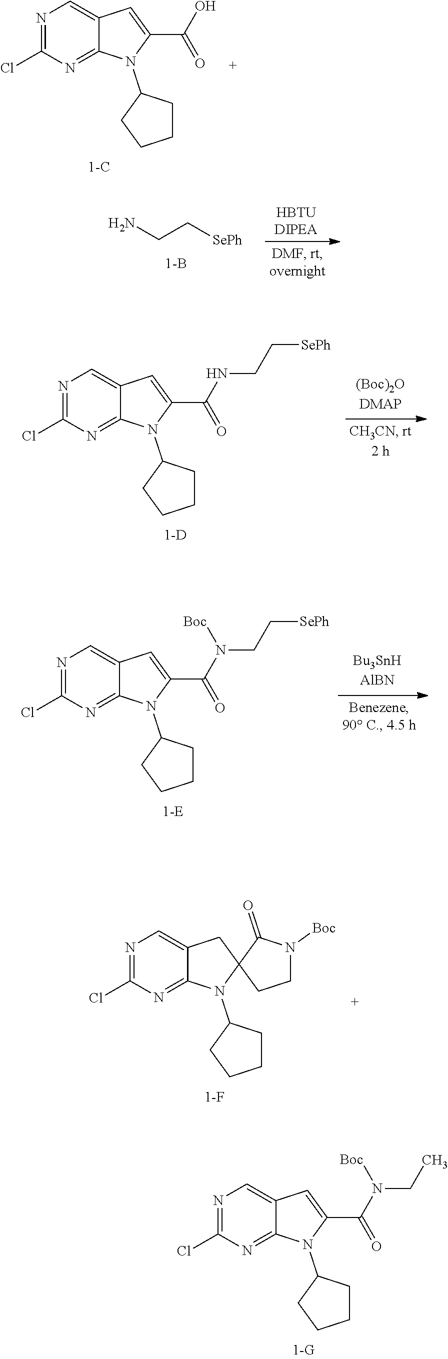

Synthesis of 1-D: (Ref: WO 2010020675): To a solution of Compound 1-C (6 g, 22.6 mmol), HBTU (12.9 g, 33.7 mmol) and diisopropylethylamine (11.8 mL, 67.8 mmol) in DMF (50 mL) was added compound 1-B (5.4 g, 27.1 mmol) and the reaction mixture was stirred for 2 h at room temperature. After completion, the reaction mixture was diluted with ethyl acetate (200 mL) and washed sequentially with saturated aqueous sodium bicarbonate (150 mL), water (100 mL), then 4× brine (100 mL). The organic phase is dried on Na₂SO₄ and concentrated to get the crude compound which is further purified by column chromatography (0 to 40% ethyl acetate in hexanes) to give compound 1-D (9.5 g, 94% yield) as a light-yellow solid. MS(ESI) m/z 449.1 (M+H)⁺. ¹H NMR (600 MHz, Chloroform-d) δ 8.79 (s, 1H), 7.58-7.54 (m, 2H), 7.31-7.28 (m, 3H), 6.61 (s, 1H), 6.58 (s, 1H), 5.46-5.40 (m, 1H), 3.73 (q, J=6.2 Hz, 2H), 3.16 (t, J=6.4 Hz, 2H), 2.42-236 (m, 2H), 2.11-2.03 (m, 4H), 1.70-1.66 (m, 2H).

Synthesis of 1-E: (Ref: Putey et al., J. Med. Chem. 52(19): 5916-5925 (2009)): A solution of amide 1-D (9.5 g, 21.2 mmol), Boc₂O (7.4 g, 33.9 mmol), and a catalytic amount of DMAP (520 mg, 4.24 mmol) in MeCN (200 mL) was stirred at room temperature for 2 h. After evaporation of the solvent, the residue was partitioned between EtOAc (100 mL) and H₂O (25 mL). The two phases were separated, and the aqueous phase was extracted with EtOAc (2×50 mL). The combined organic phases were dried over Na₂SO₄ and concentrated in vacuo. The crude residue was purified by flash chromatography (Hexane/EtOAc 4:1) to give 1-E (11 g, 95%). MS(ESI) m/z 549.1 (M+H)⁺. ¹H NMR (600 MHz, Chloroform-d) δ 8.81 (s, 1H), 7.60-7.53 (m, 2H), 7.35-7.21 (m, 3H), 6.59 (s, 1H), 5.10-4.96 (m, 1H), 4.15-4.05 (m, 2H), 3.28-3.16 (m, 2H), 2.46-2.34 (m, 2H), 2.17-2.01 (m, 4H), 1.75-1.64 (m, 2H), 1.17 (s, 9H).

Synthesis of 1-F and 1-G: (Ref: Kyei et al., Tetrahedron Lett. 45(48): 8931-8934 (2004)): A solution of tributyltin hydride (4.4 mL, 16.3 mmol) and 2,2′-azobis(isobutyronitrile) (361 mg, 20 mole percent) in benzene (100 mL) was added drop wise to a solution of 1-E (6 g, 10.9 mmol) in benzene (100 mL) at 90° C. over 1.5 hours. The reaction solution was stirred at the same temperature for three hours. The reaction solution was returned to room temperature and poured into water, followed by extraction with ethyl acetate. The combined organic extract was washed with brine, dried over sodium sulfate, and concentrated under vacuum. The residue was taken in 9:1 Hexane:Ethyl acetate solution and filtered to get pure 1-F (2.9 g). The filtrate was concentrated and purified on column (to get Compound 1-F. (3.25 g, 76%). MS(ESI) m/z 393.2 (M+H)⁺. ¹H NMR (600 MHz, Chloroform-d) δ 7.69 (s, 1H), 3.93-3.88 (m, 1H), 3.63-3.48 (m, 2H), 3.24-3.21 (m, 1H), 2.99-2.94 (m, 1H), 2.38-2.30 (m, 2H), 2.20-2.09 (m, 2H), 1.97-1.89 (m, 3H), 1.76-1.68 (m, 1H), 1.56 (s, 9H), 1.54-1.51 (m, 2H). Compound 1-G. (50 mg, 1%). MS(ESI) m/z 393.2 (M+H)⁺. ¹H NMR (600 MHz, Chloroform-d) δ 8.81 (s, 1H), 6.55 (s, 1H), 5.08-4.99 (m, 1H), 3.91-3.85 (m, 2H), 2.46-2.34 (m, 2H), 2.19-2.01 (m, 4H), 1.72-1.66 (m, 2H), 1.30 (t, J=7.1 Hz, 3H), 1.20 (s, 9H).

### Step 3: Synthesis of 7′-cyclopentyl-2′-((5-(piperazin-1-yl)pyridin-2-yl)amino)-5′,7′-dihydrospiro[pyrrolidine-3,6′-pyrrolo[2,3-d]pyrimidin]-2-one (Compound C33)

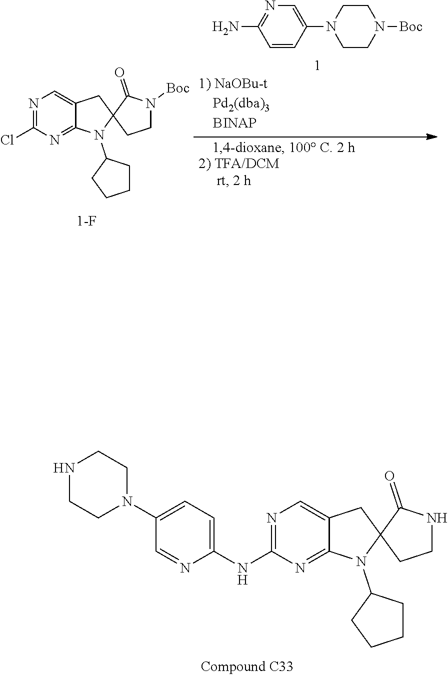 [SMILES: CC(C)(C)OC(=O)N1CCN(c2ccc(N)nc2)CC1 ; CC(C)(C)OC(=O)N1CCC2(Cc3cnc(Cl)nc3N2C2CCCC2)C1=O ; O=C1NCCC12Cc1cnc(Nc3ccc(N4CCNCC4)cn3)nc1N2C1CCCC1]

Compound C33 was synthesized following the general procedure A, 3 mg (3% yield). MS(ESI) m/z 435.3 (M+H)⁺.

### Example 2: Compound C34

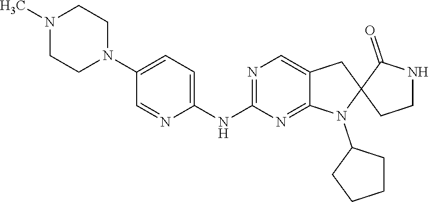 [SMILES: CN1CCN(c2ccc(Nc3ncc4c(n3)N(C3CCCC3)C3(CCNC3=O)C4)nc2)CC1]

### Synthesis of 7′-cyclopentyl-2′-((5-(4-methylpiperazin-1-yl)pyridin-2-yl)amino)-5′,7′-dihydrospiro[pyrrolidine-3,6′-pyrrolo[2,3-d]pyrimidin]-2-one (Compound C34)

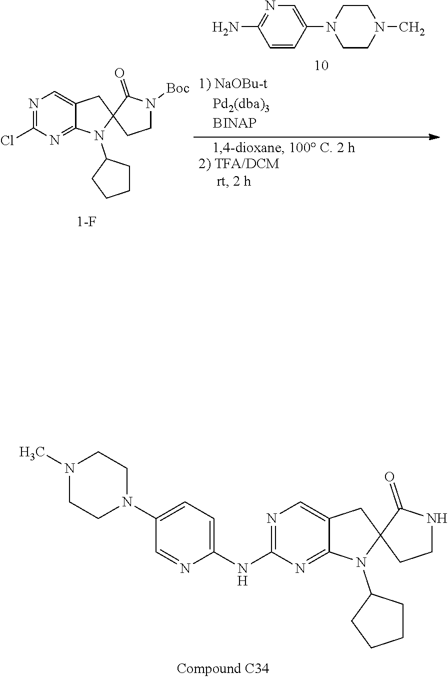 [SMILES: CN1CCN(c2ccc(N)nc2)CC1 ; CC(C)(C)OC(=O)N1CCC2(Cc3cnc(Cl)nc3N2C2CCCC2)C1=O ; CN1CCN(c2ccc(Nc3ncc4c(n3)N(C3CCCC3)C3(CCNC3=O)C4)nc2)CC1]

Compound C34 was synthesized following the general procedure A, 1 mg (1% yield). MS(ESI) m/z 449.3 (M+H)⁺.

### Example 3: Compound C38

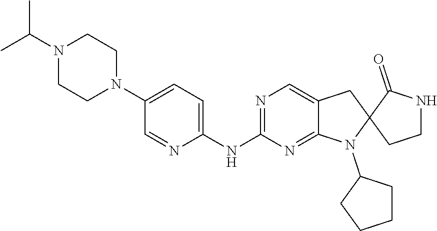 [SMILES: CC(C)N1CCN(c2ccc(Nc3ncc4c(n3)N(C3CCCC3)C3(CCNC3=O)C4)nc2)CC1]

### Synthesis of 7′-cyclopentyl-2′-((5-(4-isopropylpiperazin-1-yl)pyridin-2-yl)amino)-5′,7′-dihydrospiro[pyrrolidine-3,6′-pyrrolo[2,3-d]pyrimidin]-2-one (Compound C38)

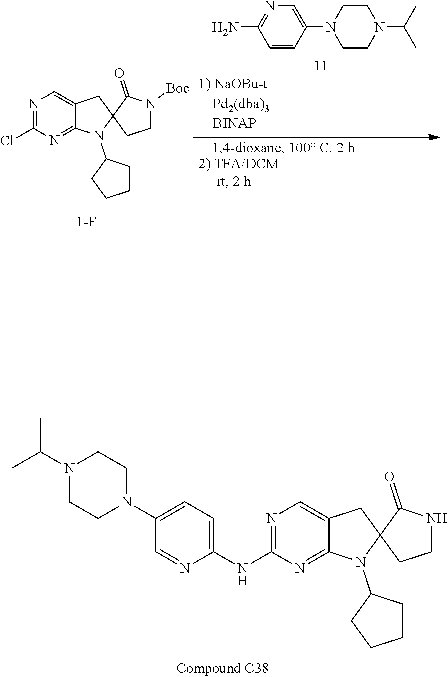 [SMILES: CC(C)N1CCN(c2ccc(N)nc2)CC1 ; CC(C)(C)OC(=O)N1CCC2(Cc3cnc(Cl)nc3N2C2CCCC2)C1=O ; CC(C)N1CCN(c2ccc(Nc3ncc4c(n3)N(C3CCCC3)C3(CCNC3=O)C4)nc2)CC1]

Compound C38 was synthesized following the general procedure A, 7 mg (2% yield). MS(ESI) m/z 477.3 (M+H)⁺.

### Example 4: Compound C43

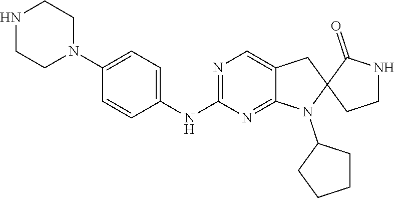 [SMILES: O=C1NCCC12Cc1cnc(Nc3ccc(N4CCNCC4)cc3)nc1N2C1CCCC1]

### Synthesis of 7′-cyclopentyl-2′-((4-(piperazin-1-yl)phenyl)amino)-5′,7′-dihydrospiro[pyrrolidine-3,6′-pyrrolo[2,3-d]pyrimidin]-2-one (Compound C43)

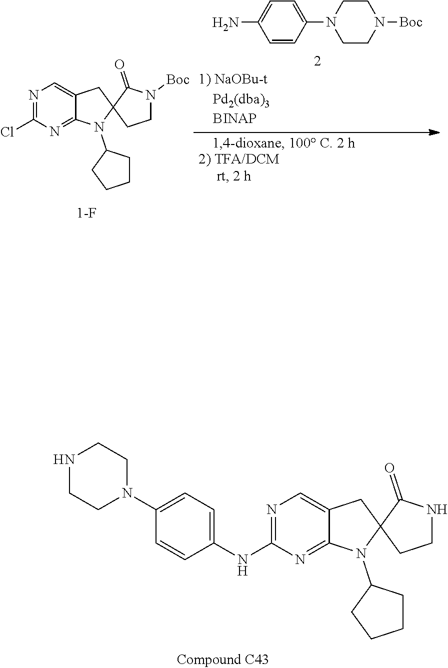 [SMILES: CC(C)(C)OC(=O)N1CCN(c2ccc(N)cc2)CC1 ; CC(C)(C)OC(=O)N1CCC2(Cc3cnc(Cl)nc3N2C2CCCC2)C1=O ; O=C1NCCC12Cc1cnc(Nc3ccc(N4CCNCC4)cc3)nc1N2C1CCCC1]

Compound C43 was synthesized following the general procedure A, 6 mg (7% yield). MS(ESI) m/z 434.3 (M+H)⁺.

### Example 5: Compound C45

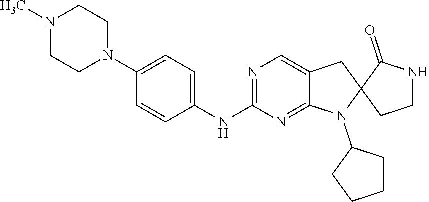 [SMILES: CN1CCN(c2ccc(Nc3ncc4c(n3)N(C3CCCC3)C3(CCNC3=O)C4)cc2)CC1]

### Synthesis of 7′-cyclopentyl-2′-((4-(4-methylpiperazin-1-yl)phenyl)amino)-5′,7′-dihydrospiro[pyrrolidine-3,6′-pyrrolo[2,3-d]pyrimidin]-2-one (Compound C45)

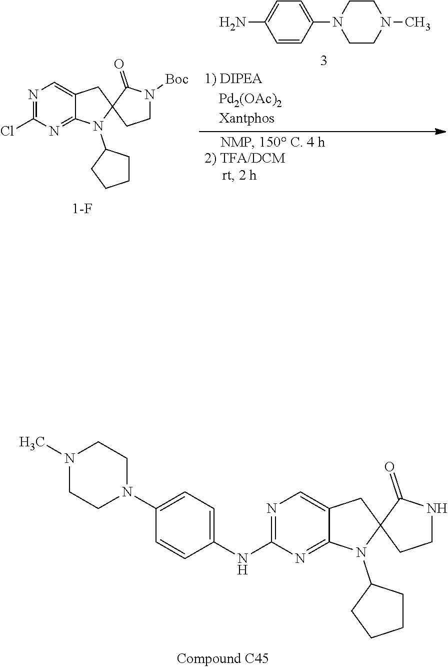 [SMILES: CN1CCN(c2ccc(N)cc2)CC1 ; CC(C)(C)OC(=O)N1CCC2(Cc3cnc(Cl)nc3N2C2CCCC2)C1=O ; CN1CCN(c2ccc(Nc3ncc4c(n3)N(C3CCCC3)C3(CCNC3=O)C4)cc2)CC1]

Compound C45 was synthesized following the general procedure 1, 11 mg (12% yield). MS(ESI) m/z 448.3 (M+H)⁺. ¹H NMR (600 MHz, Chloroform-d) δ 11.47 (s, 1H), 7.44 (d, J=9.0 Hz, 2H), 7.25 (s, 1H), 6.88 (d, J=9.0 Hz, 2H), 6.02 (s, 1H), 3.55-3.42 (m, 3H), 3.33 (s, 4H), 3.23 (dd, J=16.3, 1.7 Hz, 1H), 3.01 (s, 3H), 2.95 (d, J=17.2 Hz, 1H), 2.65 (s, 3H), 2.56-2.47 (m, 1H), 2.38-2.27 (m, 3H), 2.25-2.17 (m, 2H), 1.91-1.66 (m, 3H), 1.53-1.44 (m, 2H).

### Example 6: Compound C46

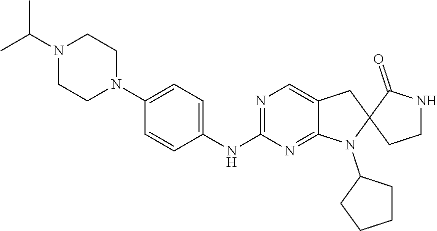 [SMILES: CC(C)N1CCN(c2ccc(Nc3ncc4c(n3)N(C3CCCC3)C3(CCNC3=O)C4)cc2)CC1]

### Synthesis of 7′-cyclopentyl-2′-((4-(4-isopropylpiperazin-1-yl)phenyl)amino)-5′,7′-dihydrospiro[pyrrolidine-3,6′-pyrrolo[2,3-d]pyrimidin]-2-one (Compound C46)

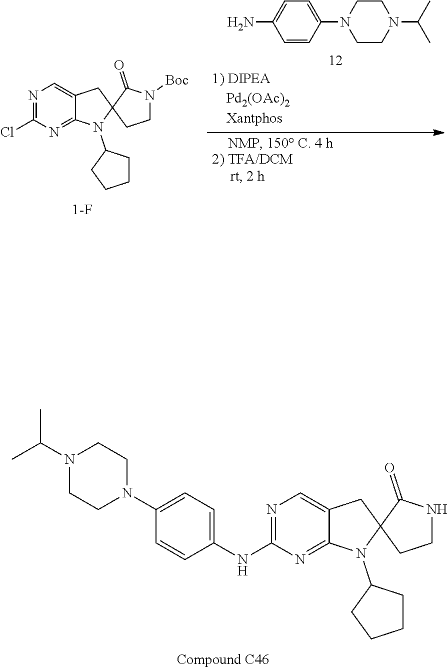 [SMILES: CC(C)N1CCN(c2ccc(N)cc2)CC1 ; CC(C)(C)OC(=O)N1CCC2(Cc3cnc(Cl)nc3N2C2CCCC2)C1=O ; CC(C)N1CCN(c2ccc(Nc3ncc4c(n3)N(C3CCCC3)C3(CCNC3=O)C4)cc2)CC1]

Compound C46 was synthesized following the general procedure I, 7 mg (7% yield). MS(ESI) m/z 476.3 (M+H)⁺. ¹H NMR (600 MHz, Chloroform-d) δ 7.44 (d, J=8.9 Hz, 2H), 7.33 (s, 1H), 6.88 (d, J=9.0 Hz, 2H), 5.95 (s, 1H), 3.52-3.39 (m, 3H), 3.25-3.17 (m, 4H), 2.92 (d, J=16.0 Hz, 1H), 2.81 (s, 3H), 2.53-2.37 (m, 3H), 2.31-2.24 (m, 4H), 1.91-1.84 (m, 1H), 1.80-1.67 (m, 3H), 1.54-1.43 (m, 2H), 1.16 (d, J=6.4 Hz, 6H).

### Example 7: Compound C47

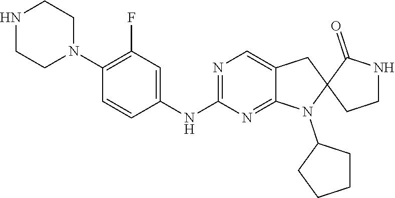 [SMILES: O=C1NCCC12Cc1cnc(Nc3ccc(N4CCNCC4)c(F)c3)nc1N2C1CCCC1]

### Synthesis of 7′-cyclopentyl-2′-((3-fluoro-4-(piperazin-1-yl)phenyl)amino)-5′,7′-dihydrospiro[pyrrolidine-3,6′-pyrrolo[2,3-d]pyrimidin]-2-one (Compound C47)

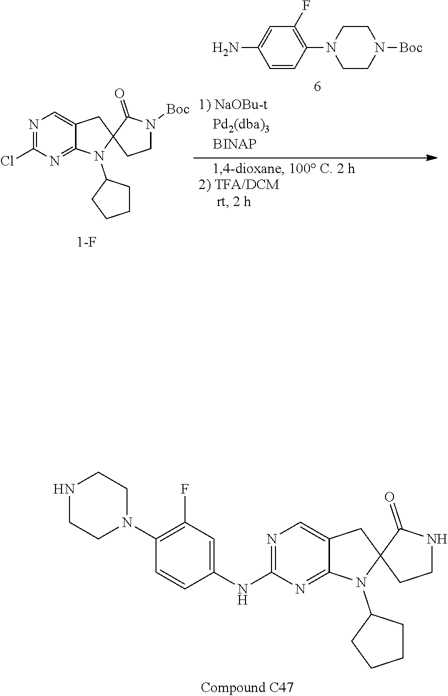 [SMILES: CC(C)(C)OC(=O)N1CCN(c2ccc(N)cc2F)CC1 ; CC(C)(C)OC(=O)N1CCC2(Cc3cnc(Cl)nc3N2C2CCCC2)C1=O ; O=C1NCCC12Cc1cnc(Nc3ccc(N4CCNCC4)c(F)c3)nc1N2C1CCCC1]

Compound C47 was synthesized following the general procedure A, 2 mg (1% yield). MS(ESI) m/z 452.3 (M+H)⁺.

### Example 8: Compound C48

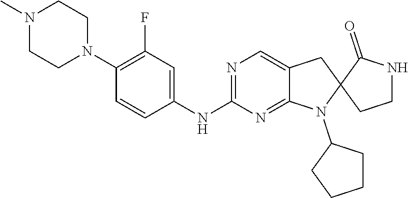 [SMILES: CN1CCN(c2ccc(Nc3ncc4c(n3)N(C3CCCC3)C3(CCNC3=O)C4)cc2F)CC1]

### Synthesis of 7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one (Compound C48)

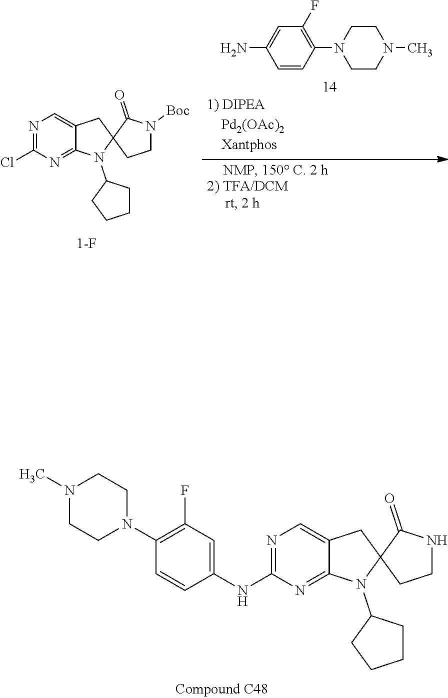 [SMILES: CN1CCN(c2ccc(N)cc2F)CC1 ; CC(C)(C)OC(=O)N1CCC2(Cc3cnc(Cl)nc3N2C2CCCC2)C1=O ; CN1CCN(c2ccc(Nc3ncc4c(n3)N(C3CCCC3)C3(CCNC3=O)C4)cc2F)CC1]

Compound C48 was synthesized following the general procedure I, 7 mg (8% yield). MS(ESI) m/z 466.3 (M+H)⁺. ¹H NMR (600 MHz, Chloroform-d) δ 9.44 (s, 1H), 7.66 (dd, J=14.7, 2.4 Hz, 1H), 7.45 (s, 1H), 7.11-7.03 (m, 1H), 6.88 (t, J=9.1 Hz, 1H), 5.94 (s, 1H), 3.52-3.39 (m, 2H), 3.20 (dd, J=15.7, 1.4 Hz, 1H), 3.09 (s, 3H), 2.92 (d, J=16.0 Hz, 1H), 2.64 (s, 3H), 2.55-2.38 (m, 7H), 2.37 (s, 3H), 2.28-2.22 (m, 1H), 1.96-1.87 (m, 2H), 1.78-1.69 (m, 1H), 1.62-1.49 (m, 2H).

### Example 9: Compound C49

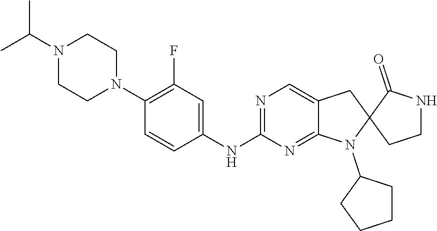 [SMILES: CC(C)N1CCN(c2ccc(Nc3ncc4c(n3)N(C3CCCC3)C3(CCNC3=O)C4)cc2F)CC1]

### Synthesis of 7′-cyclopentyl-2′-((3-fluoro-4-(4-isopropylpiperazin-1-yl)phenyl)amino)-5′,7′-dihydrospiro[pyrrolidine-3,6′-pyrrolo[2,3-d]pyrimidin]-2-one (Compound C49)

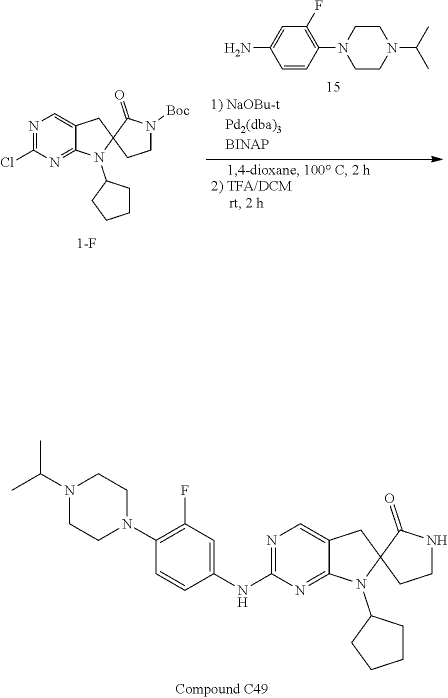 [SMILES: CC(C)N1CCN(c2ccc(N)cc2F)CC1 ; CC(C)(C)OC(=O)N1CCC2(Cc3cnc(Cl)nc3N2C2CCCC2)C1=O ; CC(C)N1CCN(c2ccc(Nc3ncc4c(n3)N(C3CCCC3)C3(CCNC3=O)C4)cc2F)CC1]

Compound C49 was synthesized following the general procedure A, 4 mg (2% yield). MS(ESI) m/z 494.3 (M+H)⁺. ¹H NMR (600 MHz, Chloroform-d) δ 7.54 (d, J=12.3 Hz, 1H), 7.34 (s, 1H), 7.15 (d, J=8.7 Hz, 1H), 6.91 (t, J=9.1 Hz, 1H), 5.87 (s, 1H), 3.55-3.41 (m, 3H), 3.30-3.20 (m, 5H), 3.03 (s, 3H), 2.96 (d, J=16.8 Hz, 2H), 2.58-2.48 (m, 1H), 2.48-2.38 (m, 1H), 2.34-2.26 (m, 2H), 1.96-1.88 (m, 2H), 1.87-1.80 (m, 3H), 1.76-1.51 (m, 2H), 1.25 (s, 6H).

### Example 10: Compound C53

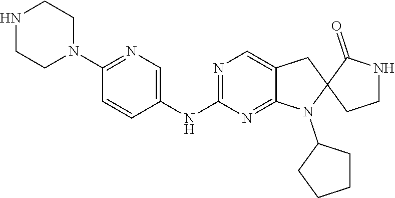 [SMILES: O=C1NCCC12Cc1cnc(Nc3ccc(N4CCNCC4)nc3)nc1N2C1CCCC1]

### Synthesis of 7′-cyclopentyl-2′-((6-(piperazin-1-yl)pyridin-3-yl)amino)-5′,7′-dihydrospiro[pyrrolidine-3,6′-pyrrolo[2,3-d]pyrimidin]-2-one (Compound C53)

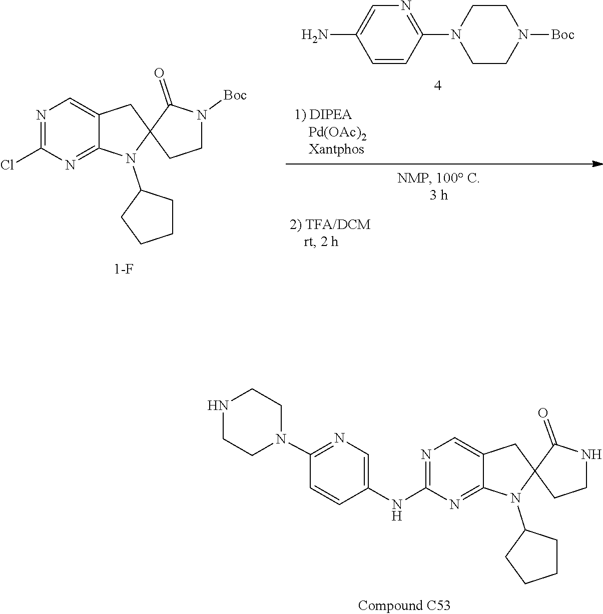 [SMILES: CC(C)(C)OC(=O)N1CCN(c2ccc(N)cn2)CC1 ; CC(C)(C)OC(=O)N1CCC2(Cc3cnc(Cl)nc3N2C2CCCC2)C1=O ; O=C1NCCC12Cc1cnc(Nc3ccc(N4CCNCC4)nc3)nc1N2C1CCCC1]

Compound C53 was synthesized following the general procedure I, 2 mg (2% yield). MS(ESI) m/z 435.3 (M+H)⁺.

### Example 11: Compound C54

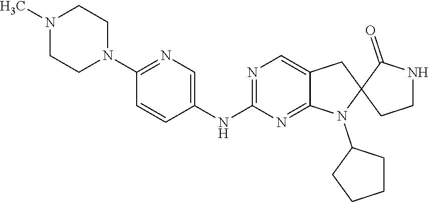 [SMILES: CN1CCN(c2ccc(Nc3ncc4c(n3)N(C3CCCC3)C3(CCNC3=O)C4)cn2)CC1]

### Synthesis of 7′-cyclopentyl-2′-((6-(4-methylpiperazin-1-yl)pyridin-3-yl)amino)-5′,7′-dihydrospiro[pyrrolidine-3,6′-pyrrolo[2,3-d]pyrimidin]-2-one (Compound C54)

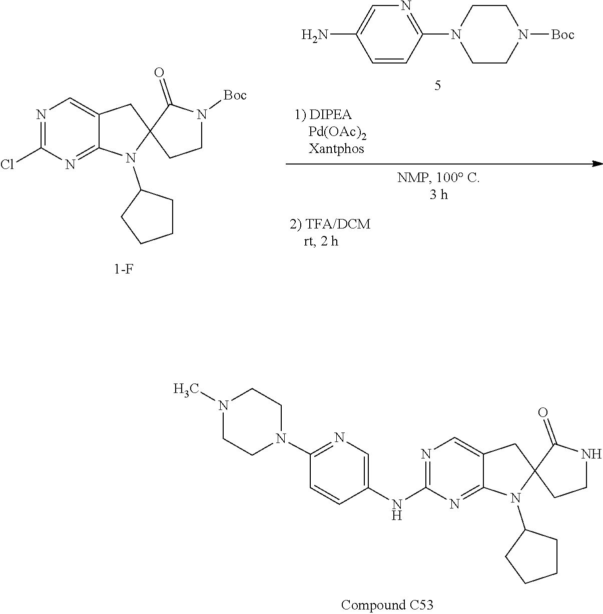 [SMILES: CC(C)(C)OC(=O)N1CCN(c2ccc(N)cn2)CC1 ; CC(C)(C)OC(=O)N1CCC2(Cc3cnc(Cl)nc3N2C2CCCC2)C1=O ; CN1CCN(c2ccc(Nc3ncc4c(n3)N(C3CCCC3)C3(CCNC3=O)C4)cn2)CC1]

Compound C54 was synthesized following the general procedure I, 4 mg. MS(ESI) m/z 449.3 (M+H)⁺.

### Example 12: Compound C55

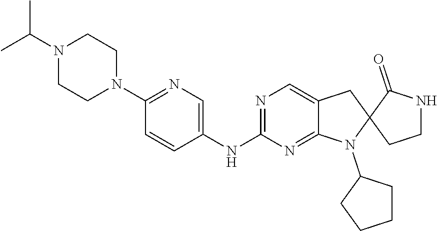 [SMILES: CC(C)N1CCN(c2ccc(Nc3ncc4c(n3)N(C3CCCC3)C3(CCNC3=O)C4)cn2)CC1]

### Synthesis of 7′-cyclopentyl-2′-((6-(4-isopropylpiperazin-1-yl)pyridin-3-yl)amino)-5′,7′-dihydrospiro[pyrrolidine-3,6′-pyrrolo[2,3-d]pyrimidin]-2-one (Compound C55)

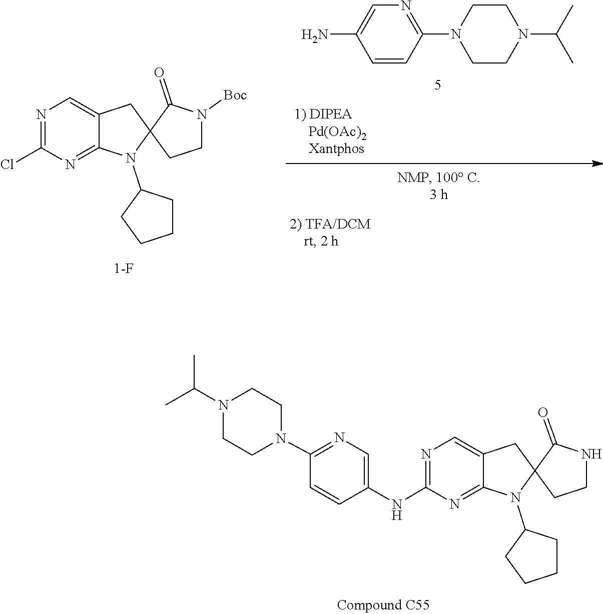 [SMILES: CC(C)N1CCN(c2ccc(N)cn2)CC1 ; CC(C)(C)OC(=O)N1CCC2(Cc3cnc(Cl)nc3N2C2CCCC2)C1=O ; CC(C)N1CCN(c2ccc(Nc3ncc4c(n3)N(C3CCCC3)C3(CCNC3=O)C4)cn2)CC1]

Compound C55 was synthesized following the general procedure 1, 14 mg (15% yield). MS(ESI) m/z 477.3 (M+H)⁺. ¹H NMR (600 MHz, Chloroform-d) δ 11.62 (s, 1H), 8.37 (d, J=2.4 Hz, 1H), 7.60 (dd, J=8.9, 2.3 Hz, 1H), 7.25 (s, 1H), 6.65 (d, J=8.9 Hz, 1H), 5.89 (s, 1H), 3.54-3.41 (m, 4H), 3.24 (dd, J=16.3, 1.7 Hz, 1H), 2.99-2.93 (m, 1H), 2.56-2.48 (m, 1H), 2.34-2.20 (m, 2H), 2.17-2.08 (m, 1H), 1.91-1.81 (m, 1H), 1.78-1.69 (m, 2H), 1.58 (s, 8H), 1.49-1.41 (m, 3H), 1.34 (d, J=5.3 Hz, 6H).

### Example 13: Compound C50

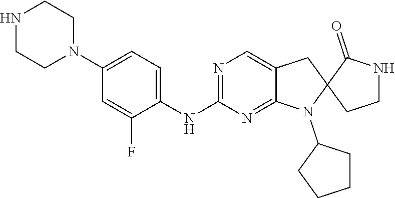 [SMILES: O=C1NCCC12Cc1cnc(Nc3ccc(N4CCNCC4)cc3F)nc1N2C1CCCC1]

### Synthesis of 7′-cyclopentyl-2′-((2-fluoro-4-(piperazin-1-yl)phenyl)amino)-5′,7′-dihydrospiro[pyrrolidine-3,6′-pyrrolo[2,3-d]pyrimidin]-2-one (Compound C50)

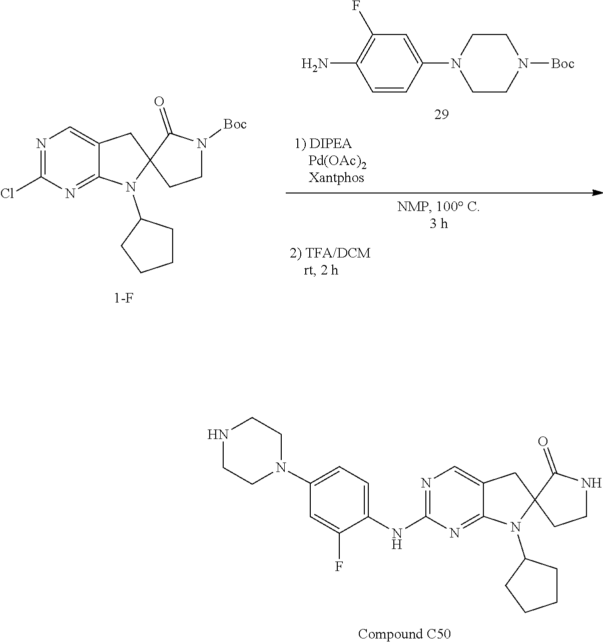 [SMILES: CC(C)(C)OC(=O)N1CCN(c2ccc(N)c(F)c2)CC1 ; CC(C)(C)OC(=O)N1CCC2(Cc3cnc(Cl)nc3N2C2CCCC2)C1=O ; O=C1NCCC12Cc1cnc(Nc3ccc(N4CCNCC4)cc3F)nc1N2C1CCCC1]

Compound C50 was synthesized following the general procedure I, 3 mg (3% yield). MS(ESI) m/z 452.3 (M+H)⁺.

### Example 14: Compound C51

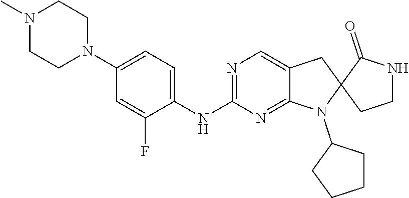 [SMILES: CN1CCN(c2ccc(Nc3ncc4c(n3)N(C3CCCC3)C3(CCNC3=O)C4)c(F)c2)CC1]

### Synthesis of 7′-cyclopentyl-2′-((2-fluoro-4-(4-methylpiperazin-1-yl)phenyl)amino)-5′,7′-dihydrospiro[pyrrolidine-3,6′-pyrrolo[2,3-d]pyrimidin]-2-one (Compound C51)

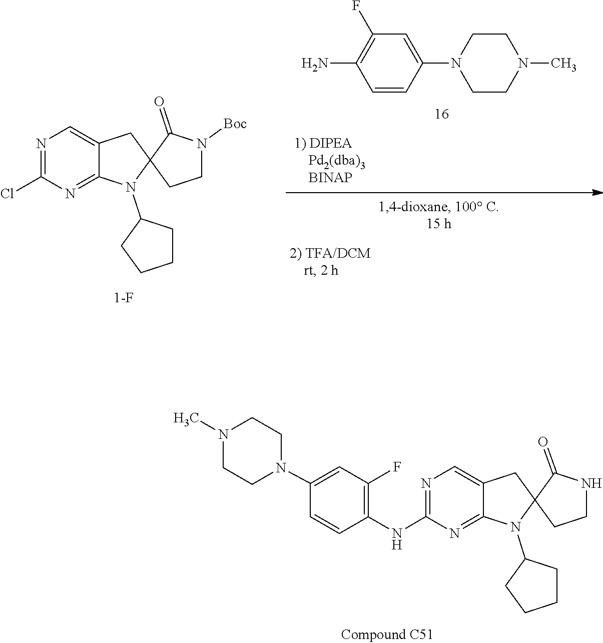 [SMILES: CN1CCN(c2ccc(N)c(F)c2)CC1 ; CC(C)(C)OC(=O)N1CCC2(Cc3cnc(Cl)nc3N2C2CCCC2)C1=O ; CN1CCN(c2ccc(Nc3ncc4c(n3)N(C3CCCC3)C3(CCNC3=O)C4)c(F)c2)CC1]

Compound C51 was synthesized following the general procedure A, 13 mg (14% yield). MS(ESI) m/z 466.3 (M+H)⁺. ¹H NMR (600 MHz, Chloroform-d) δ 10.03 (s, 1H), 7.80 (dd, J=8.5, 5.9 Hz, 1H), 7.35 (s, 1H), 6.88 (dd, J=9.4, 2.7 Hz, 1H), 6.85-6.78 (m, 1H), 6.44 (s, 1H), 3.58-3.36 (m, 7H), 3.27 (dd, J=16.4, 1.5 Hz, 1H), 3.17 (s, 3H), 2.99 (d, J=16.4 Hz, 1H), 2.81 (s, 3H), 2.57-2.49 (m, 1H), 2.35-2.23 (m, 2H), 2.23-2.14 (m, 1H), 1.86-1.62 (m, 5H), 1.58-1.49 (m, 1H), 1.50-1.41 (m, 1H).

### Example 15: Compound C52

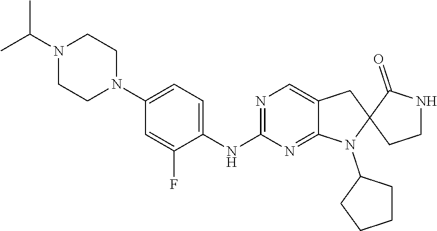 [SMILES: CC(C)N1CCN(c2ccc(Nc3ncc4c(n3)N(C3CCCC3)C3(CCNC3=O)C4)c(F)c2)CC1]

### Synthesis of 7′-cyclopentyl-2′-((2-fluoro-4-(4-isopropylpiperazin-1-yl)phenyl)amino)-5′,7′-dihydrospiro[pyrrolidine-3,6′-pyrrolo[2,3-d]pyrimidin]-2-one (Compound C52)

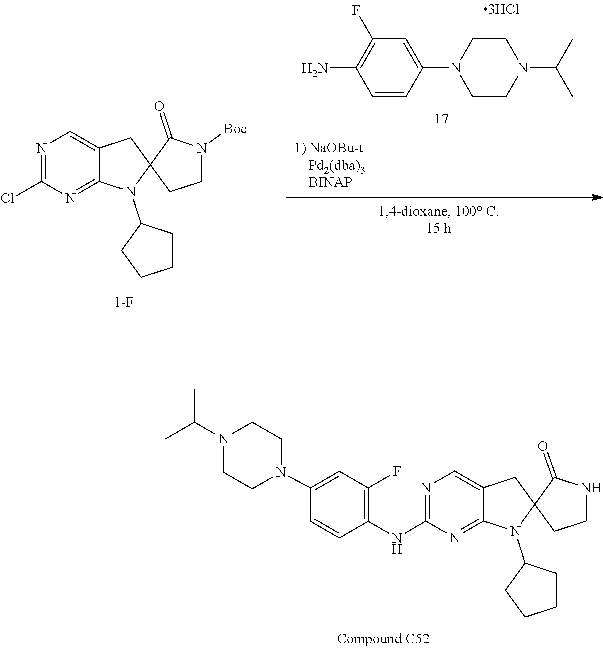 [SMILES: CC(C)N1CCN(c2ccc(N)c(F)c2)CC1 ; CC(C)(C)OC(=O)N1CCC2(Cc3cnc(Cl)nc3N2C2CCCC2)C1=O ; CC(C)N1CCN(c2ccc(Nc3ncc4c(n3)N(C3CCCC3)C3(CCNC3=O)C4)c(F)c2)CC1]

Compound C52 was synthesized following the general procedure A, 11 mg (5% yield). MS(ESI) m/z 494.3 (M+H)⁺. ¹H NMR (600 MHz, Chloroform-d) δ 10.35 (s, 1H), 7.63 (dd, J=8.8, 5.8 Hz, 1H), 7.30 (s, 1H), 6.87 (dd, J=9.5, 2.7 Hz, 1H), 6.84-6.77 (m, 1H), 6.02 (s, 1H), 3.60-3.33 (m, 10H), 3.28 (dd, J=16.5, 1.6 Hz, 1H), 3.15 (s, 2H), 3.00 (d, J=16.5 Hz, 1H), 2.61-2.50 (m, 1H), 2.37-2.30 (m, 1H), 2.29-2.18 (m, 1H), 2.16-2.07 (m, 1H), 1.91-1.82 (m, 1H), 1.80-1.71 (m, 2H), 1.70-1.63 (m, 3H), 1.42-1.35 (m, 6H).

### Example 16: Compound C56

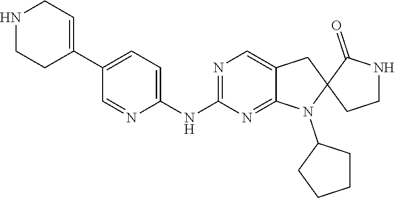 [SMILES: O=C1NCCC12Cc1cnc(Nc3ccc(C4=CCNCC4)cn3)nc1N2C1CCCC1]

### 7′-cyclopentyl-2′-((1′,2′,3′,6′-tetrahydro-[3,4′-bipyridin]-6-yl)amino)-5′,7′-dihydrospiro[pyrrolidine-3,6′-pyrrolo[2,3-d]pyrimidin]-2-one (Compound C56)

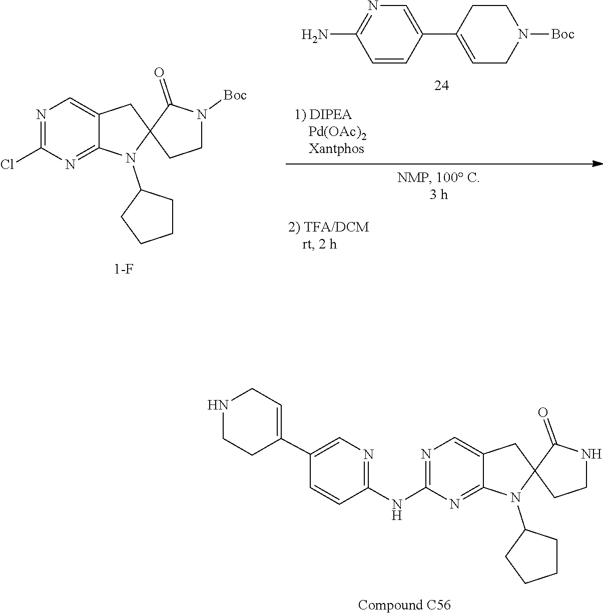 [SMILES: CC(C)(C)OC(=O)N1CC=C(c2ccc(N)nc2)CC1 ; CC(C)(C)OC(=O)N1CCC2(Cc3cnc(Cl)nc3N2C2CCCC2)C1=O ; O=C1NCCC12Cc1cnc(Nc3ccc(C4=CCNCC4)cn3)nc1N2C1CCCC1]

Compound C56 was synthesized following the general procedure I, 19 mg (22% yield). MS(ESI) m/z 432.3 (M+H)⁺.

### Example 17: Compound C57

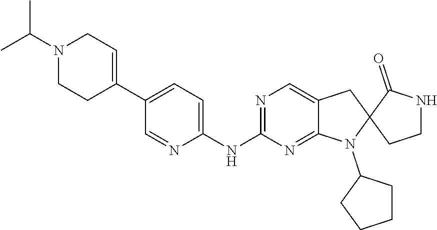 [SMILES: CC(C)N1CC=C(c2ccc(Nc3ncc4c(n3)N(C3CCCC3)C3(CCNC3=O)C4)nc2)CC1]

### Synthesis of 7′-cyclopentyl-2′-((1′-isopropyl-1′,2′,3′,6′-tetrahydro-[3,4′-bipyridin]-6-yl)amino)-5′,7′-dihydrospiro[pyrrolidine-3,6′-pyrrolo[2,3-d]pyrimidin]-2-one (Compound C57)

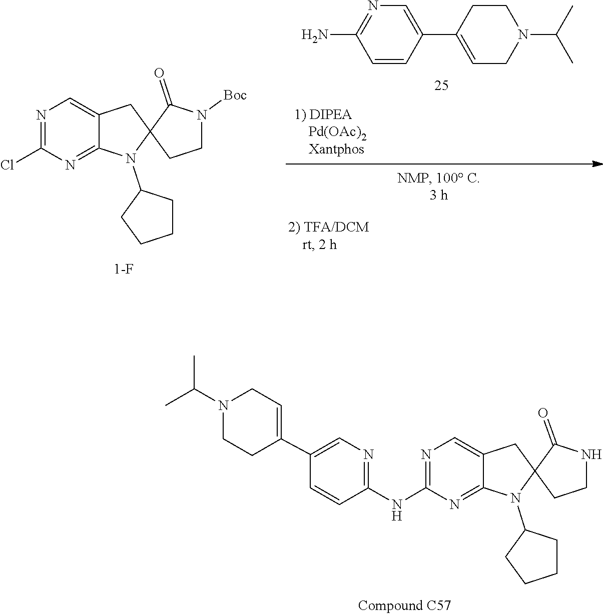 [SMILES: CC(C)N1CC=C(c2ccc(N)nc2)CC1 ; CC(C)(C)OC(=O)N1CCC2(Cc3cnc(Cl)nc3N2C2CCCC2)C1=O ; CC(C)N1CC=C(c2ccc(Nc3ncc4c(n3)N(C3CCCC3)C3(CCNC3=O)C4)nc2)CC1]

Compound C57 was synthesized following the general procedure I, 26 mg (27% yield). MS(ESI) m/z 474.3 (M+H)⁺. ¹H NMR (600 MHz, Chloroform-d) δ 8.30 (s, 1H), 7.63 (d, J=7.1 Hz, 1H), 7.46 (s, 1H), 7.26 (s, 1H), 6.05 (s, 1H), 3.74-3.62 (m, 2H), 3.61-3.43 (m, 4H), 3.22 (d, J=16.7 Hz, 1H), 2.99 (d, J=16.6 Hz, 1H), 2.59-2.50 (m, 1H), 2.41-2.21 (m, 3H), 1.91 (s, 4H), 1.84-1.72 (m, 2H), 1.59 (s, 4H), 1.43 (d, J=6.4 Hz, 6H).

### Example 18: Compound C58

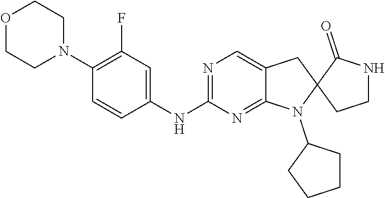 [SMILES: O=C1NCCC12Cc1cnc(Nc3ccc(N4CCOCC4)c(F)c3)nc1N2C1CCCC1]

### Synthesis of 7′-cyclopentyl-2′-((3-fluoro-4-morpholinophenyl)amino)-5′,7′-dihydrospiro[pyrrolidine-3,6′-pyrrolo[2,3-d]pyrimidin]-2-one (Compound C58)

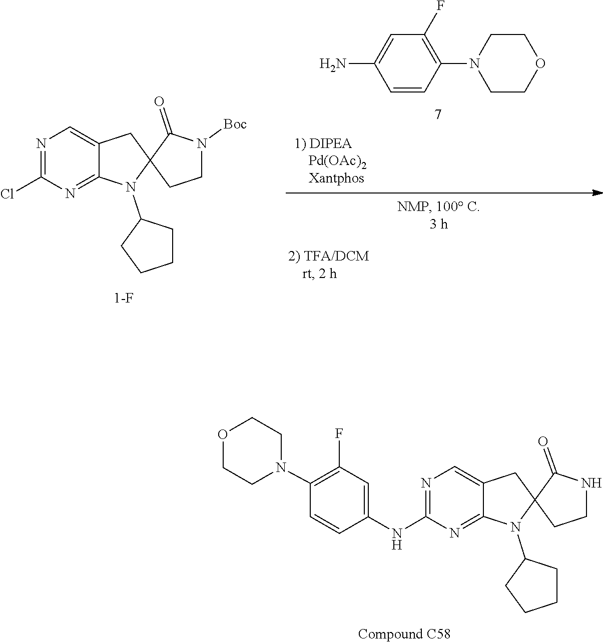 [SMILES: Nc1ccc(N2CCOCC2)c(F)c1 ; CC(C)(C)OC(=O)N1CCC2(Cc3cnc(Cl)nc3N2C2CCCC2)C1=O ; O=C1NCCC12Cc1cnc(Nc3ccc(N4CCOCC4)c(F)c3)nc1N2C1CCCC1]

Compound C58 was synthesized following the general procedure 1, 12 mg (13% yield). MS(ESI) m/z 453.3.3 (M+H)⁺. ¹H NMR (600 MHz, Chloroform-d) δ 11.36 (s, 1H), 7.55 (dd, J=14.3, 2.4 Hz, 1H), 7.29 (s, 1H), 7.20-7.12 (m, 1H), 6.89 (t, J=9.1 Hz, 1H), 6.01 (s, 1H), 3.94-3.83 (m, 4H), 3.58-3.41 (m, 3H), 3.24 (dd, J=16.2, 1.6 Hz, 1H), 3.09-3.02 (m, 4H), 2.97 (d, J=17.1 Hz, 1H), 2.58-2.48 (m, 1H), 2.49-2.36 (m, 1H), 2.36-2.25 (m, 2H), 1.97-1.88 (m, 1H), 1.88-1.81 (m, 2H), 1.81-1.73 (m, 1H), 1.59-1.47 (m, 2H).

### Example 19: Compound C59

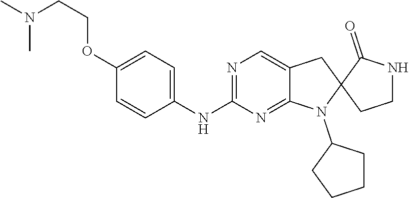 [SMILES: CN(C)CCOc1ccc(Nc2ncc3c(n2)N(C2CCCC2)C2(CCNC2=O)C3)cc1]

### Synthesis of 7′-cyclopentyl-2′-((4-(2-(dimethylamino)ethoxy)phenyl)amino)-5′,7′-dihydrospiro[pyrrolidine-3,6′-pyrrolo[2,3-d]pyrimidin]-2-one (Compound C59)

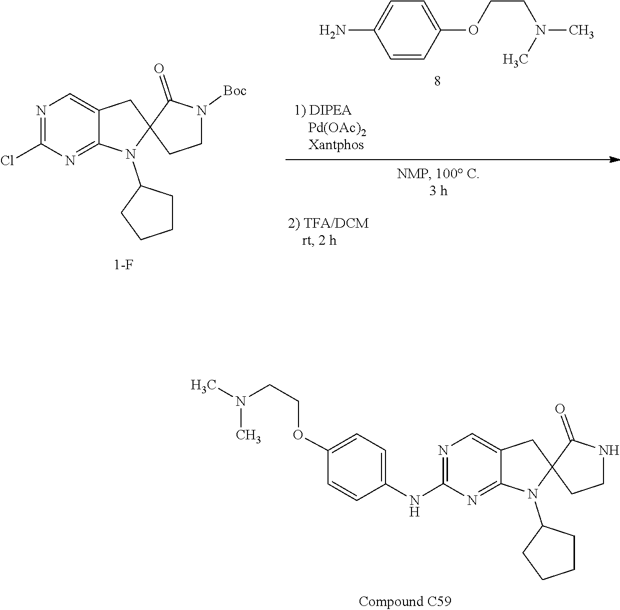 [SMILES: CN(C)CCOc1ccc(N)cc1 ; CC(C)(C)OC(=O)N1CCC2(Cc3cnc(Cl)nc3N2C2CCCC2)C1=O ; CN(C)CCOc1ccc(Nc2ncc3c(n2)N(C2CCCC2)C2(CCNC2=O)C3)cc1]

Compound C59 was synthesized following the general procedure I, 9 mg (10% yield). MS(ESI) m/z 437.3 (M+H)⁺. ¹H NMR (600 MHz, Chloroform-d) δ 11.26 (s, 1H), 7.45 (d, J=8.9 Hz, 2H), 7.30 (s, 1H), 6.86 (d, J=9.0 Hz, 2H), 6.33 (s, 1H), 4.17 (t, J=5.3 Hz, 2H), 3.53-3.40 (m, 3H), 3.25-3.18 (m, 1H), 3.07-2.99 (m, 2H), 2.94 (d, J=16.5 Hz, 1H), 2.56 (s, 6H), 2.53-2.44 (m, 1H), 2.40-2.32 (m, 1H), 2.32-2.18 (m, 2H), 1.91-1.81 (m, 1H), 1.78-1.67 (m, 3H), 1.55-1.41 (m, 2H).

### Example 20: Compound C60

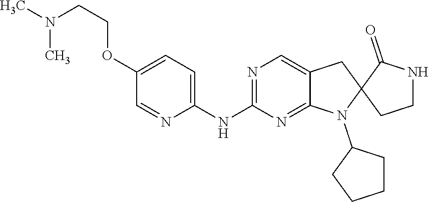 [SMILES: CN(C)CCOc1ccc(Nc2ncc3c(n2)N(C2CCCC2)C2(CCNC2=O)C3)nc1]

### Synthesis of 7′-cyclopentyl-2′-((5-(2-(dimethylamino)ethoxy)pyridin-2-yl)amino)-5′,7′-dihydrospiro[pyrrolidine-3,6′-pyrrolo[2,3-d]pyrimidin]-2-one (Compound C60)

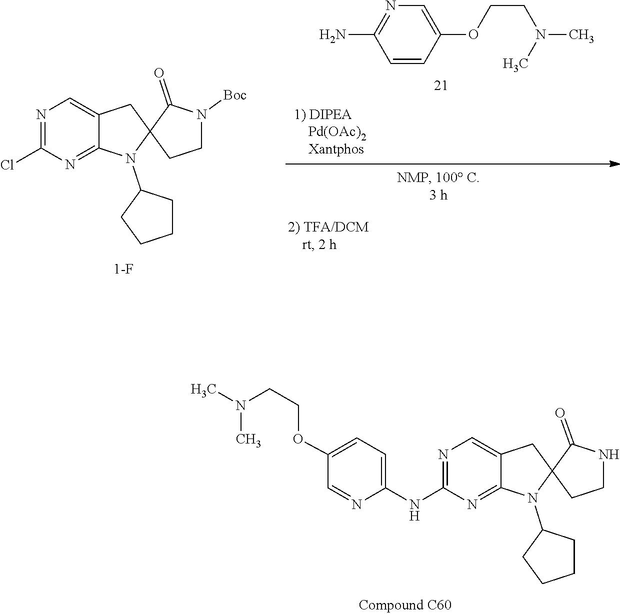 [SMILES: CN(C)CCOc1ccc(N)nc1 ; CC(C)(C)OC(=O)N1CCC2(Cc3cnc(Cl)nc3N2C2CCCC2)C1=O ; CN(C)CCOc1ccc(Nc2ncc3c(n2)N(C2CCCC2)C2(CCNC2=O)C3)nc1]

Compound C60 was synthesized following the general procedure I, 17 mg (19% yield). MS(ESI) m/z 438.3 (M+H)⁺.

### Example 21: Compound C61

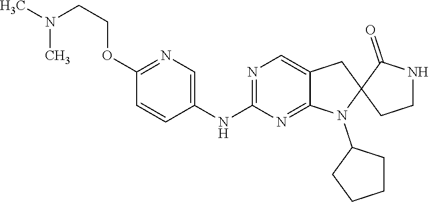 [SMILES: CN(C)CCOc1ccc(Nc2ncc3c(n2)N(C2CCCC2)C2(CCNC2=O)C3)cn1]

### Synthesis of 7′-cyclopentyl-2′-((6-(2-(dimethylamino)ethoxy)pyridin-3-yl)amino)-5′,7′-dihydrospiro[pyrrolidine-3,6′-pyrrolo[2,3-d]pyrimidin]-2-one_(Compound C61)

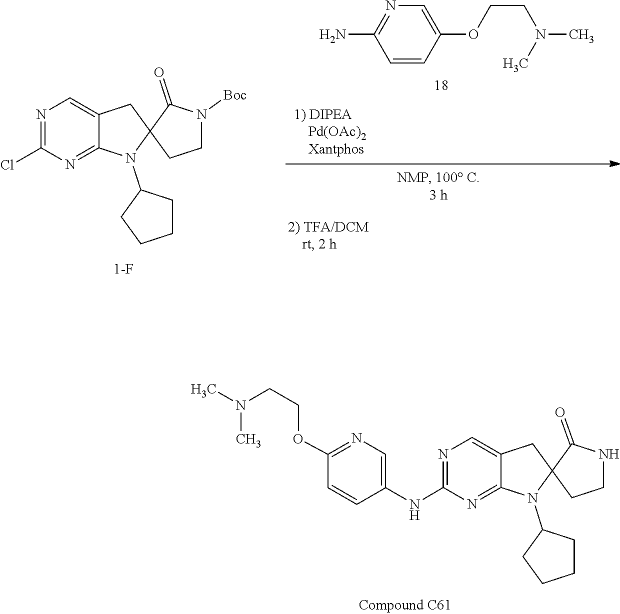 [SMILES: CN(C)CCOc1ccc(N)nc1 ; CC(C)(C)OC(=O)N1CCC2(Cc3cnc(Cl)nc3N2C2CCCC2)C1=O ; CN(C)CCOc1ccc(Nc2ncc3c(n2)N(C2CCCC2)C2(CCNC2=O)C3)cn1]

Compound C61 was synthesized following the general procedure I, 7 mg (8% yield). MS(ESI) m/z 438.3 (M+H)⁺.

### Example 22: Compound C62

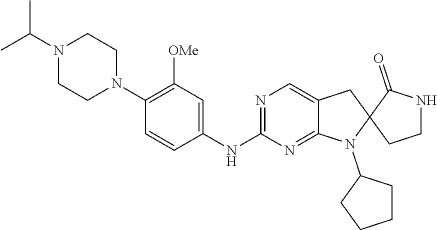 [SMILES: COc1cc(Nc2ncc3c(n2)N(C2CCCC2)C2(CCNC2=O)C3)ccc1N1CCN(C(C)C)CC1]

### Synthesis of 7′-cyclopentyl-2′-((4-(4-isopropylpiperazin-1-yl)-3-methoxyphenyl)amino)-5′,7′-dihydrospiro[pyrrolidine-3,6′-pyrrolo[2,3-d]pyrimidin]-2-one (Compound C62)

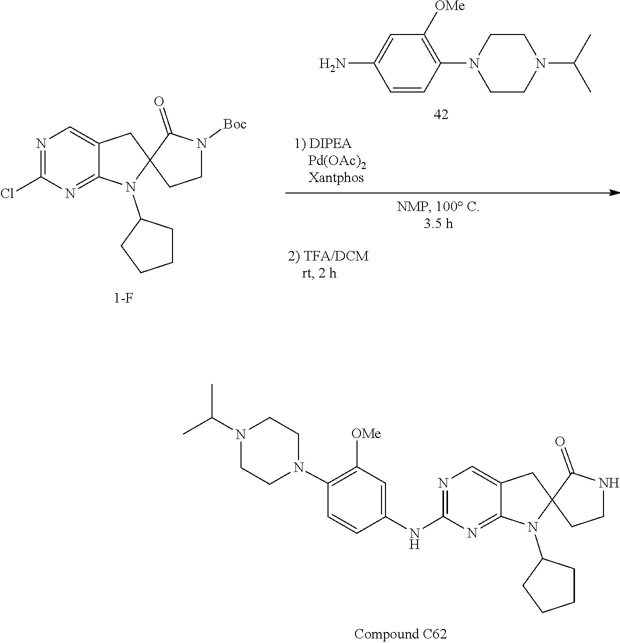 [SMILES: COc1cc(N)ccc1N1CCN(C(C)C)CC1 ; CC(C)(C)OC(=O)N1CCC2(Cc3cnc(Cl)nc3N2C2CCCC2)C1=O ; COc1cc(Nc2ncc3c(n2)N(C2CCCC2)C2(CCNC2=O)C3)ccc1N1CCN(C(C)C)CC1]

Compound C62 was synthesized following the general procedure I, 24 mg (24% yield). MS(ESI) m/z 506.3 (M+H)⁺. ¹H NMR (600 MHz, Chloroform-d) δ 11.36 (s, 1H), 7.27 (s, 1H), 7.20 (dd, J=8.5, 2.1 Hz, 1H), 6.91-6.85 (m, 2H), 6.00 (s, 1H), 3.87 (s, 3H), 3.56-3.41 (m, 5H), 3.34 (s, 4H), 3.23 (dd, J=16.3, 1.7 Hz, 2H), 2.96 (d, J=17.2 Hz, 1H), 2.56-2.48 (m, 1H), 2.34-2.24 (m, 2H), 2.21-2.10 (m, 1H), 1.91-1.83 (m, 2H), 1.79-1.69 (m, 3H), 1.50-1.40 (m, 3H), 1.38 (d, J=6.6 Hz, 6H).

### Example 23: Compound C63

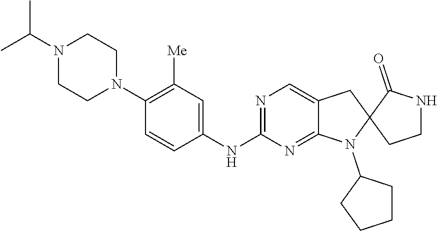 [SMILES: Cc1cc(Nc2ncc3c(n2)N(C2CCCC2)C2(CCNC2=O)C3)ccc1N1CCN(C(C)C)CC1]

### Synthesis of 7′-cyclopentyl-2′-((4-(4-isopropylpiperazin-1-yl)-3-methylphenyl)amino)-5′,7′-dihydrospiro[pyrrolidine-3,6′-pyrrolo[2,3-d]pyrimidin]-2-one (Compound C63)

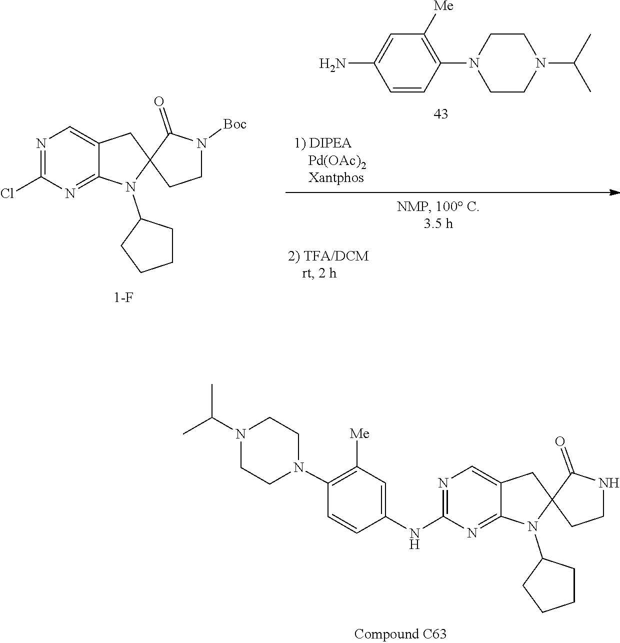 [SMILES: Cc1cc(N)ccc1N1CCN(C(C)C)CC1 ; CC(C)(C)OC(=O)N1CCC2(Cc3cnc(Cl)nc3N2C2CCCC2)C1=O ; Cc1cc(Nc2ncc3c(n2)N(C2CCCC2)C2(CCNC2=O)C3)ccc1N1CCN(C(C)C)CC1]

Compound C63 was synthesized following the general procedure I, 32 mg (33% yield). MS(ESI) m/z 490.3 (M+H)⁺. ¹H NMR (600 MHz, Chloroform-d) δ 10.93 (s, 1H), 7.39 (dd, J=8.5, 2.0 Hz, 1H), 7.30 (s, 1H), 7.25 (d, J=2.2 Hz, 1H), 7.00 (d, J=8.6 Hz, 1H), 6.06 (s, 1H), 3.54-3.40 (m, 3H), 3.22 (dd, J=16.1, 1.5 Hz, 1H), 3.12 (s, 5H), 2.94 (d, J=16.8 Hz, 1H), 2.56-2.46 (m, 1H), 2.39-2.29 (m, 2H), 2.28 (s, 3H), 2.25-2.16 (m, 2H), 1.93-1.80 (m, 2H), 1.78-1.62 (m, 4H), 1.54-1.40 (m, 3H), 1.31 (d, J=6.2 Hz, 6H).

### Example 24: Compound C17

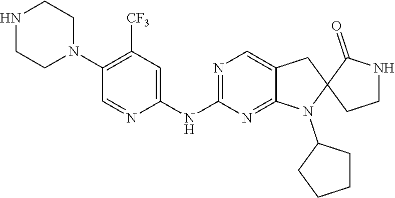 [SMILES: O=C1NCCC12Cc1cnc(Nc3cc(C(F)(F)F)c(N4CCNCC4)cn3)nc1N2C1CCCC1]

### Synthesis of 7′-cyclopentyl-2′-((5-(piperazin-1-yl)-4-(trifluoromethyl)pyridin-2-yl)amino)-5′,7′-dihydrospiro[pyrrolidine-3,6′-pyrrolo[2,3-d]pyrimidin]-2-one_(Compound C17)

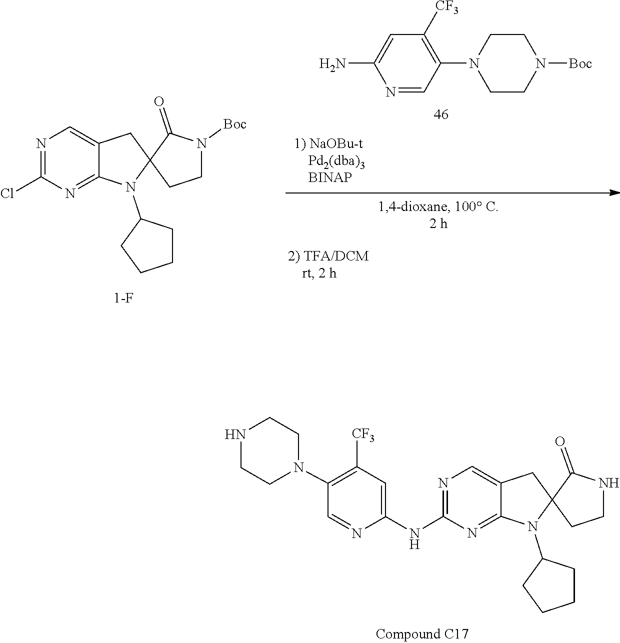 [SMILES: CC(C)(C)OC(=O)N1CCN(c2cnc(N)cc2C(F)(F)F)CC1 ; CC(C)(C)OC(=O)N1CCC2(Cc3cnc(Cl)nc3N2C2CCCC2)C1=O ; O=C1NCCC12Cc1cnc(Nc3cc(C(F)(F)F)c(N4CCNCC4)cn3)nc1N2C1CCCC1]

Compound C17 was synthesized following the general procedure A, 16 mg (13% yield). MS(ESI) m/z 503.3 (M+H)⁺.

### Example 25: Compound C18

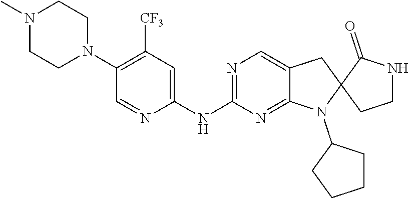 [SMILES: CN1CCN(c2cnc(Nc3ncc4c(n3)N(C3CCCC3)C3(CCNC3=O)C4)cc2C(F)(F)F)CC1]

### Synthesis of 7′-cyclopentyl-2′-((5-(4-methylpiperazin-1-yl)-4-(trifluoromethyl)pyridin-2-yl)amino)-5′,7′-dihydrospiro[pyrrolidine-3,6′-pyrrolo[2,3-d]pyrimidin]-2-one (Compound C18)

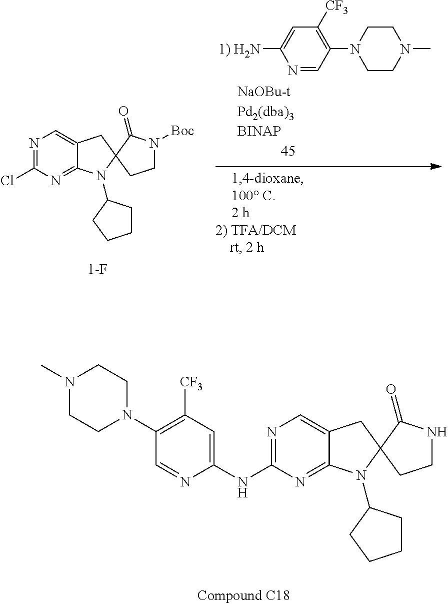

Compound C18 was synthesized following the general procedure A, 14 mg (12% yield). MS(ESI) m/z 517.3 (M+H)⁺.

### Example 26: Compound C95

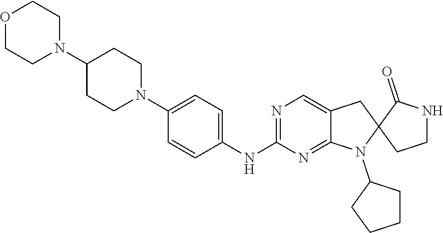 [SMILES: O=C1NCCC12Cc1cnc(Nc3ccc(N4CCC(N5CCOCC5)CC4)cc3)nc1N2C1CCCC1]

### Synthesis of 4-chloro-2-(methylthio)pyrimidine-5-carbaldehyde (26-C)

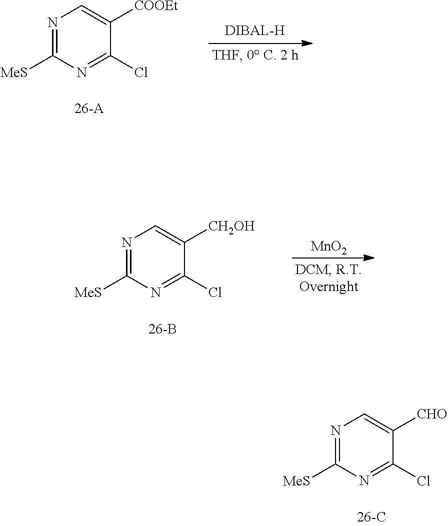 [SMILES: CCOC(=O)c1cnc(SC)nc1Cl ; CSc1ncc(CO)c(Cl)n1 ; "MnO2" ; CSc1ncc(C=O)c(Cl)n1]

Synthesis of 27-B: (Ref: WO 2015161285): To a stirred solution of compound 26-A (40 g, 172 mmol) in anhydrous THF (500 mL) at 0° C. was added DIBAL-H (516 mL, 3 mmol) dropwise and stirred at 0° C. for 1 h and quenched with 600 mL of saturated NH₄Cl at 0° C. and warmed to RT and added 600 mL of saturated Rochelle Salt. To this, added 600 mL of ethyl acetate and stirred for 30 minutes. Two layers separated, and aqueous layer was washed with ethyl acetate and combined organic layers dried on Na₂SO₄ and concentrated to get the crude compound which is purified by column chromatography (0 to 40% ethyl acetate in hexanes) to give compound 26-B (27 g, 67% yield). MS(ESI) m/z 191.1 [M+H]⁺.

Synthesis of 26-C: (Ref: Reddy et al., J. Med. Chem. 57(3): 578-599 (2014)): The compound 26-B (20 g, 105 mmol) was dissolved in dichloromethane to which manganese dioxide (MnO₂) (137 g, 1572 mmol) was added and stirred for 48 h. The solids were removed by filtration through a Celite pad and washed with dichloromethane. Solvent evaporated under reduced pressure to get crude compound, which was purified by column chromatography (0 to 20% ethyl acetate in hexanes) to give compound 26-C (13.3 g, 68% yield) as yellow solid. MS(ESI) m/z 189.0 [M+H]⁺.

### Step 2: Synthesis of ethyl 2-(cyclopentylamino)acetate hydrochloride (26-G)

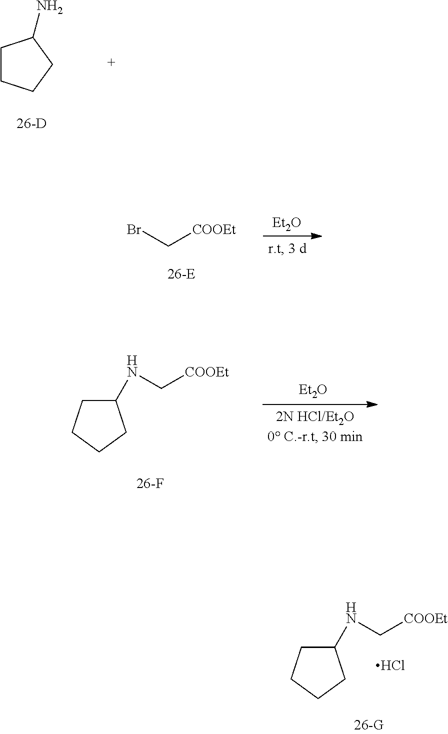

Synthesis of 26-F: (Ref: WO 199948858): To a solution of compound 26-D (6 g, 70.46 mmol) in ether (100 mL) was added compound 26-E (5.9 g, 35.23 mmol) and stirred at room temperature for 3 days. The solid was filtered and the filtrate was concentrated to get the crude product 26-F.

Synthesis of 26-G: Compound 26-F was dissolved in ether and treated with 2 N HCl/Et₂O (2 eq). The obtained solid is filtered and dried to get 26-G (6.4 g, 89% yield) MS(ESI) m/z 172.2 [M+H]⁺. ¹H NMR (600 MHz, DMSO-d₆) δ 9.37 (s, 2H), 4.22 (q, J=7.1 Hz, 2H), 3.95 (s, 2H), 3.46 (p, J=7.3 Hz, 1H), 2.04-1.86 (m, 2H), 1.79-1.59 (m, 4H), 1.59-1.42 (m, 2H), 1.25 (t, J=7.1 Hz, 3H).

Step 3: Synthesis of 7-cyclopentyl-2-(methylthio)-7H-pyrrolo[2,3-d]pyrimidine-6-carboxylic acid (26-1)

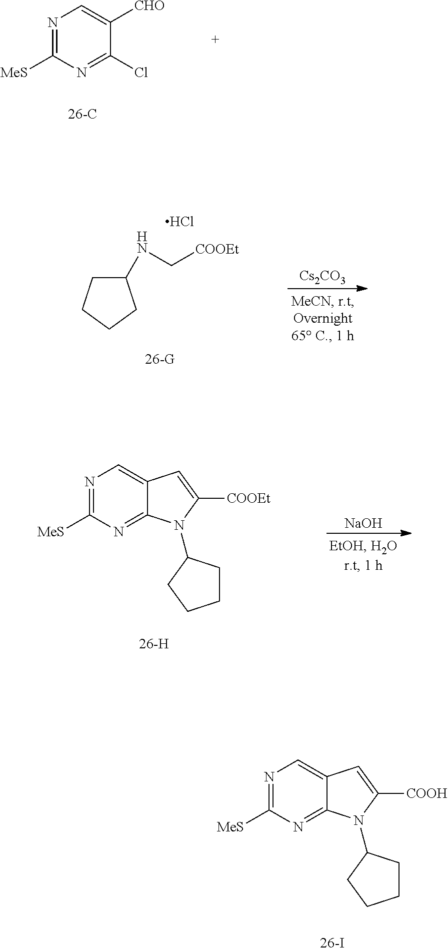

Synthesis of 26-H: To a solution of compound 26-C (2.5 g, 8 mmol) and Cs₂CO₃ (7.9 g, 24 mmol) in acetonitrile (100 mL) was added compound 26-G (1.5 g, 8 mmol) under argon and the reaction mixture was stirred for overnight at RT and then at 65° C. for 1 h. After cooling to RT, solvent is removed under reduced pressure and partitioned between with ethyl acetate (50 mL) and water (30 mL). The organic layer is separated, and aqueous layer was washed 3 times with ethyl acetate. Combined organic layers were dried on Na₂SO₄ and concentrated to get the crude compound which is purified by column chromatography (0 to 10% ethyl acetate in hexanes) to give compound 26-H (1.6 g, 67% yield) as a white solid. MS(ESI) m/z 306.1 [M+H]⁺. ¹HNMR (600 MHz, Chloroform-d) δ 8.81 (s, 1H), 7.22 (s, 1H), 5.82 (p, J=8.7 Hz, 1H), 4.38 (q, J=7.1 Hz, 2H), 2.62 (s, 3H), 2.56-2.46 (m, 2H), 2.13-2.01 (m, 4H), 1.77-1.66 (m, 2H), 1.41 (t, J=7.1 Hz, 3H).

Synthesis of 26-I: To a solution of compound 26-H (1.6 g, 5.2 mmol) in ethanol (20 mL) was added 10 mL of aqueous 2N NaOH and stirred at room temperature for 1 h. The Solvent removed under reduced pressure, diluted with 10 mL of water and acidified with 2 N HCl. The solid was filtered and dried to give 26-1 as a white solid (1.4 g, 98% yield). MS(ESI) m/z 278.1 [M+H]⁺. ¹H NMR (600 MHz, DMSO-d₆) δ 13.35 (s, 1H), 8.96 (s, 1H), 7.28 (s, 1H), 5.81 (p, J=8.7 Hz, 1H), 2.57 (s, 3H), 2.45-2.36 (m, 2H), 2.07-1.95 (m, 4H), 1.72-1.61 (m, 2H).

### Step 4: Synthesis of tert-butyl 2′-chloro-7′-cyclopentyl-2-oxo-5′,7′-dihydrospiro[pyrrolidine-3,6′-pyrrolo[2,3-d]pyrimidine]-1-carboxylate (26-L)

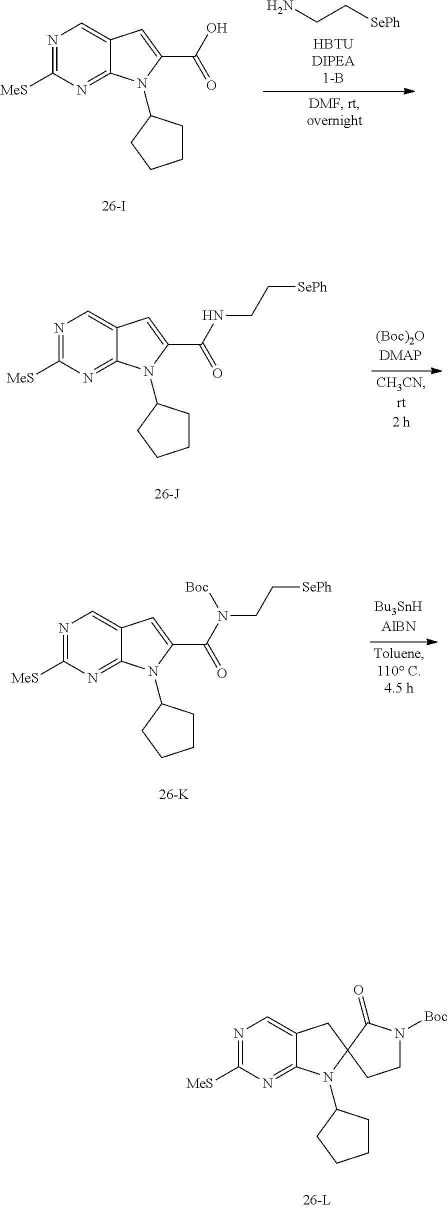

Synthesis of 26-J: Prepared using the procedure described for the synthesis of compound 1-D. (20.5 g, 94% yield). MS(ESI) m/z 462.2 (M+H)⁺. ¹HNMR (600 MHz, Chloroform-d) δ 8.73 (s, 1H), 7.60-7.55 (m, 2H), 7.32-7.27 (m, 3H), 6.53 (s, 1H), 5.47 (p, J=8.7 Hz, 1H), 3.72 (q, J=6.2 Hz, 2H), 3.15 (q, J=6.2 Hz, 2H), 2.61 (s, 3H), 2.55-2.43 (m, 2H), 2.12-1.94 (m, 4H), 1.72-1.63 (m, 2H).

Synthesis of 26-K: Prepared using the procedure described for the synthesis of compound 1-E. (21 g, 80% yield). MS(ESI) m/z 561.1 (M+H)⁺. ¹HNMR (600 MHz, Chloroform-d) δ 8.75 (d, J=1.3 Hz, 1H), 7.60-7.52 (m, 2H), 7.32-7.25 (m, 3H), 6.56 (s, 1H), 4.99 (p, J=8.8 Hz, 1H), 4.12-4.04 (m, 2H), 3.25-3.19 (m, 2H), 2.62 (s, 3H), 2.56-2.45 (m, 2H), 2.15-2.01 (m, 4H), 1.75-1.64 (m, 2H), 1.18 (s, 9H).

Synthesis of 26-L: Prepared using the procedure described for the synthesis of compound 1-F and 1-G. (8.4 g, 58% yield). MS(ESI) m/z 405.1 (M+H)⁺. ¹H NMR (600 MHz, Chloroform-d) δ 7.71 (s, 1H), 3.94-3.83 (m, 1H), 3.64-3.52 (m, 1H), 3.51-3.42 (m, 1H), 3.19 (dd, J=16.0, 1.2 Hz, 1H), 2.93 (d, J=16.1 Hz, 1H), 2.48 (s, 3H), 2.47-2.43 (m, 1H), 2.33-2.23 (m, 2H), 2.12-2.07 (m, 1H), 1.94-1.86 (m, 3H), 1.73-1.65 (m, 1H), 1.57 (s, 9H), 1.54-1.48 (m, 2H).

### Step 5: Synthesis of tert-butyl 2′-chloro-7′-cyclopentyl-2-oxo-5′,7′-dihydrospiro[pyrrolidine-3,6′-pyrrolo[2,3-d]pyrimidine]-1-carboxylate (26-M)

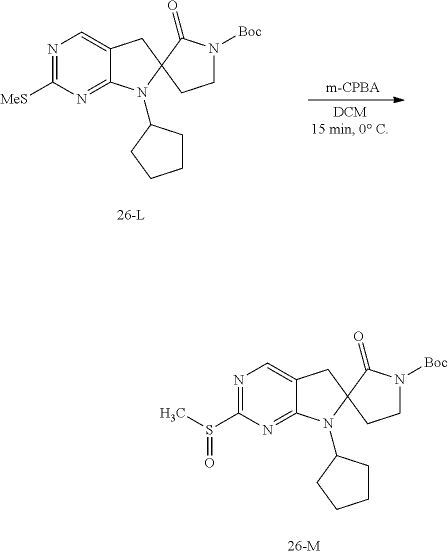 [SMILES: CSc1ncc2c(n1)N(C1CCCC1)C1(CCN(C(=O)OC(C)(C)C)C1=O)C2 ; CS(=O)c1ncc2c(n1)N(C1CCCC1)C1(CCN(C(=O)OC(C)(C)C)C1=O)C2]

To a solution of 26-L (2 g, 4.9 mmol) in DCM (400 mL) at 0° C. was added m-CPBA in one portion. The resulting mixture was stirred at same temperature for 15 min. The reaction mixture was washed with saturated NaHCO₃ solution and brine, respectively. The organic layer is then dried over sodium sulfate, filtered and concentrated under reduced pressure to give 26-M, which was used as such for next step (2 g, quantitative). MS(ESI) m/z 421.2 (M+H)⁺. ¹H NMR (600 MHz, Chloroform-d) δ 7.98 (d, J=10.1 Hz, 1H), 3.99-3.87 (m, 1H), 3.67-3.49 (m, 2H), 3.30 (d, J=17.3 Hz, 1H), 3.05 (dd, J=16.8, 3.0 Hz, 1H), 2.86 (s, 3H), 2.44-2.23 (m, 2H), 2.21-2.10 (m, 2H), 2.00-1.85 (m, 3H), 1.81-1.69 (m, 1H), 1.66 (s, 2H), 1.56 (s, 9H).

### Step 6: Synthesis of 7′-cyclopentyl-2′-((4-(4-morpholinopiperidin-1-yl)phenyl)amino)-5′,7′-dihydrospiro[pyrrolidine-3,6′-pyrrolo[2,3-d]pyrimidin]-2-one (Compound C95)

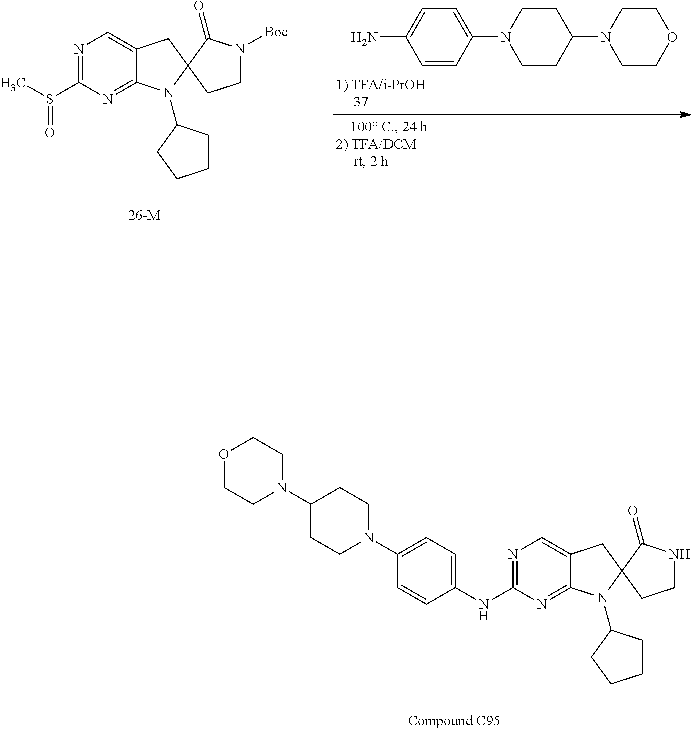 [SMILES: CS(=O)c1ncc2c(n1)N(C1CCCC1)C1(CCN(C(=O)OC(C)(C)C)C1=O)C2 ; Nc1ccc(N2CCC(N3CCOCC3)CC2)cc1 ; O=C1NCCC12Cc1cnc(Nc3ccc(N4CCC(N5CCOCC5)CC4)cc3)nc1N2C1CCCC1]

Compound C95 was synthesized following the general procedure C, 15 mg (8% yield). MS(ESI) m/z 518.3 (M+H)⁺.

### Example 27: Compound C96

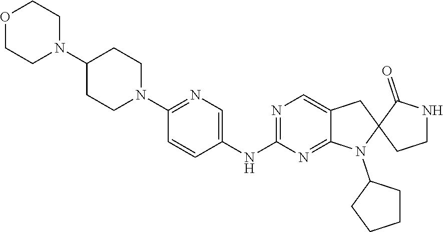 [SMILES: O=C1NCCC12Cc1cnc(Nc3ccc(N4CCC(N5CCOCC5)CC4)nc3)nc1N2C1CCCC1]

### Synthesis of 7′-cyclopentyl-2′-((6-(4-morpholinopiperidin-1-yl)pyridin-3-yl)amino)-5′,7′-dihydrospiro[pyrrolidine-3,6′-pyrrolo[2,3-d]pyrimidin]-2-one (Compound C96)

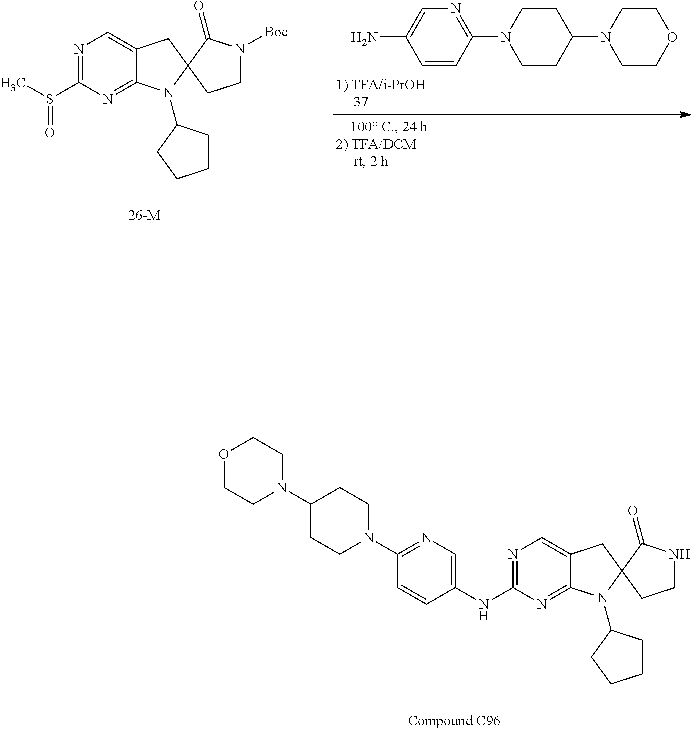 [SMILES: CS(=O)c1ncc2c(n1)N(C1CCCC1)C1(CCN(C(=O)OC(C)(C)C)C1=O)C2 ; Nc1ccc(N2CCC(N3CCOCC3)CC2)nc1 ; O=C1NCCC12Cc1cnc(Nc3ccc(N4CCC(N5CCOCC5)CC4)nc3)nc1N2C1CCCC1]

Compound C96 was synthesized following the general procedure C, 3 mg (5% yield). MS(ESI) m/z 519.3 (M+H)⁺.

### Example 28: Compound C97

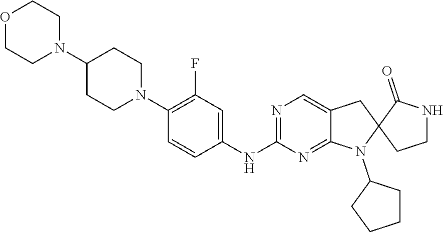 [SMILES: O=C1NCCC12Cc1cnc(Nc3ccc(N4CCC(N5CCOCC5)CC4)c(F)c3)nc1N2C1CCCC1]

### Synthesis of 7′-cyclopentyl-2′-((3-fluoro-4-(4-morpholinopiperidin-1-yl)phenyl)amino)-5′,7′-dihydrospiro[pyrrolidine-3,6′-pyrrolo[2,3-d]pyrimidin]-2-one (Compound C97)

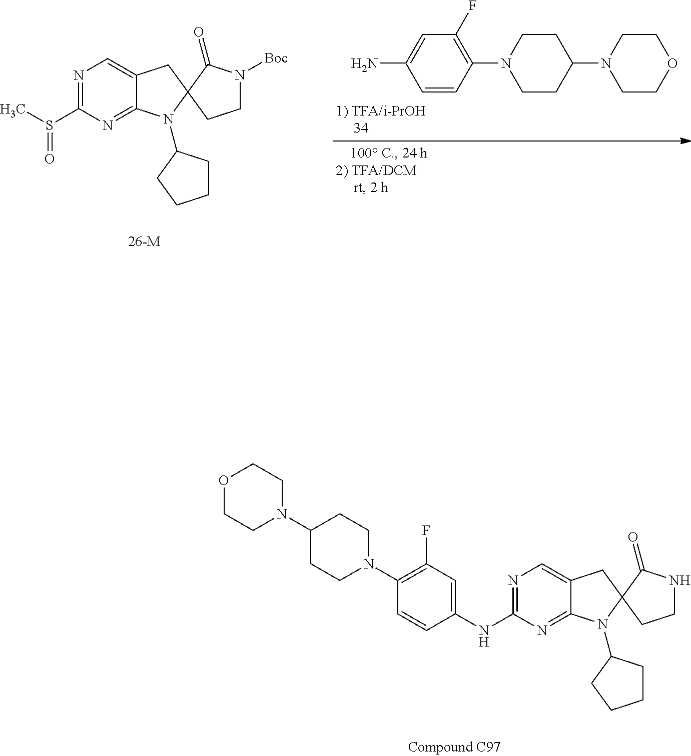 [SMILES: CS(=O)c1ncc2c(n1)N(C1CCCC1)C1(CCN(C(=O)OC(C)(C)C)C1=O)C2 ; Nc1ccc(N2CCC(N3CCOCC3)CC2)c(F)c1 ; O=C1NCCC12Cc1cnc(Nc3ccc(N4CCC(N5CCOCC5)CC4)c(F)c3)nc1N2C1CCCC1]

Compound C97 was synthesized following the general procedure C, 6 mg (9% yield). MS(ESI) m/z 536.3 (M+H)⁺.

### Example 29: Compound C107

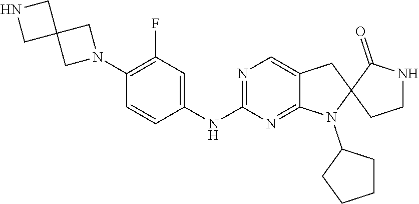 [SMILES: O=C1NCCC12Cc1cnc(Nc3ccc(N4CC5(CNC5)C4)c(F)c3)nc1N2C1CCCC1]

### Synthesis of 7′-cyclopentyl-2′-((3-fluoro-4-(2,6-diazaspiro[3.3]heptan-2-yl)phenyl)amino)-5′,7′-dihydrospiro[pyrrolidine-3,6′-pyrrolo[2,3-d]pyrimidin]-2-one (Compound C107)

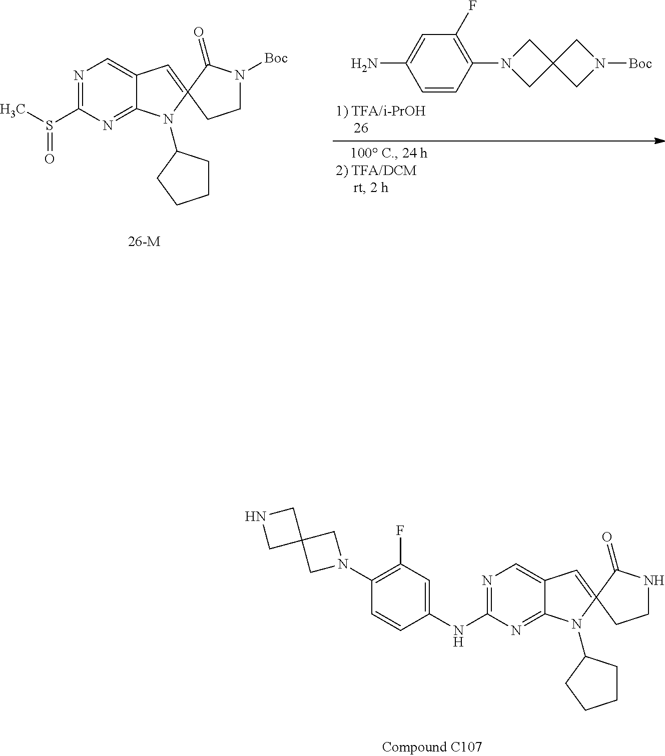

Compound C107 was synthesized following the general procedure C, 2 mg (4% yield). MS(ESI) m/z 464.3 (M+H)⁺.

### Example 30: Compound C35

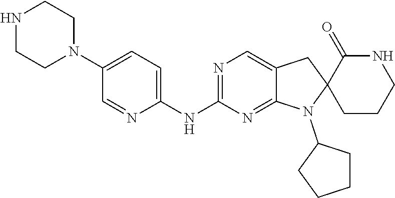 [SMILES: O=C1NCCCC12Cc1cnc(Nc3ccc(N4CCNCC4)cn3)nc1N2C1CCCC1]

### Step 1: Synthesis of 3-(phenylselanyl)propan-1-amine (30B)

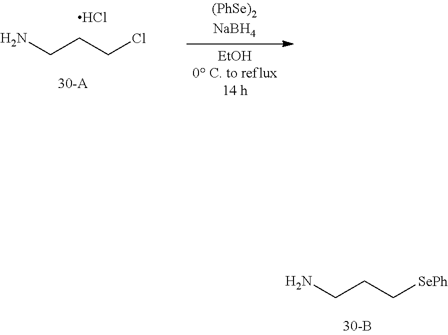

Synthesis of 30-B: Prepared using the procedure described for the synthesis of compound 1-B. (22 g, 89% yield). MS(ESI) m/z 216.1 (M+H)⁺. ¹H NMR (600 MHz, Chloroform-d) δ 7.52-7.48 (m, 2H), 7.30-7.19 (m, 3H), 2.96 (t, J=7.3 Hz, 2H), 2.81 (t, J=6.9 Hz, 2H), 1.82-1.80 (m, 2H), 1.80 (s, 2H).

### Step 2: Synthesis of tert-butyl 2′-chloro-7′-cyclopentyl-2-oxo-5′,7′-dihydrospiro[piperidine-3,6′-pyrrolo[2,3-d]pyrimidine]-1-carboxylate (30-E)

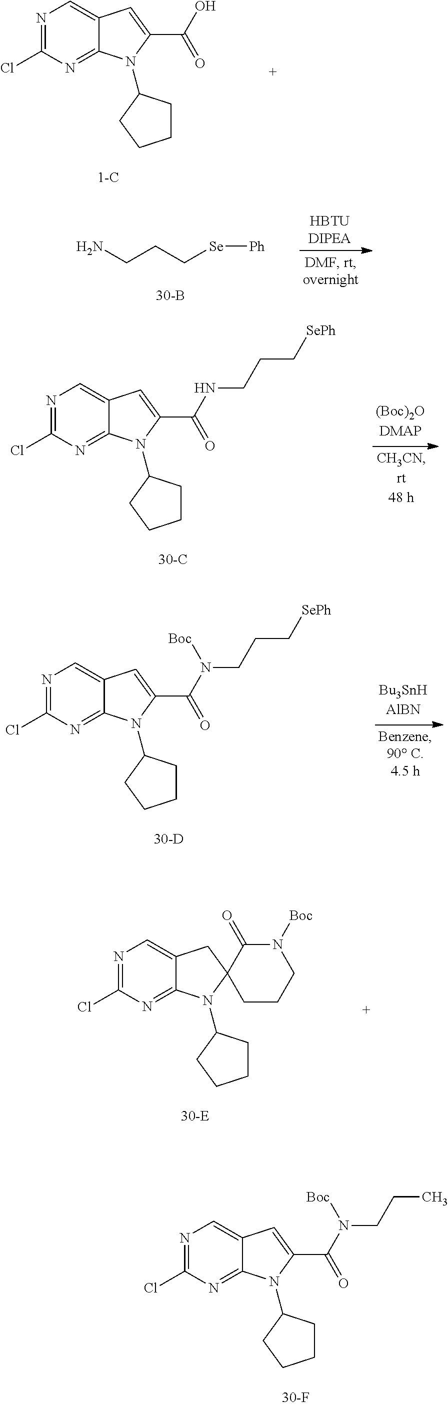

Synthesis of 30-C: Prepared using the procedure described for the synthesis of compound 1-D. (9.5 g, 91% yield). MS(ESI) m/z 463.1 (M+H)⁺. ¹H NMR (600 MHz, Chloroform-d) δ 8.80 (s, 1H), 7.57-7.49 (m, 2H), 7.35-7.22 (m, 3H), 6.68 (s, 1H), 6.35 (s, 1H), 5.50-5.39 (m, 1H), 3.58 (m, 2H), 3.00 (t, J=7.0 Hz, 2H), 2.45-2.34 (m, 2H), 2.13-2.02 (m, 6H), 1.66-1.64 (m, 2H).

Synthesis of 30-D: Prepared using the procedure described for the synthesis of compound 1-E. (10 g, 87%). MS(ESI) m/z 563.1 (M+H)⁺. ¹H NMR (600 MHz, Chloroform-d) δ 8.81 (s, 1H), 7.51-7.49 (m, 2H), 7.33-7.17 (m, 3H), 6.52 (s, 1H), 5.02 (t, J=8.8 Hz, 1H), 3.92 (t, J=7.1 Hz, 2H), 2.95 (t, J=7.4 Hz, 2H), 2.45-2.33 (m, 2H), 2.17-1.97 (m, 6H), 1.72-1.63 (m, 2H), 1.17 (s, 9H).

Synthesis of 30-E and 30-F: Prepared using the procedure described for the synthesis of compound 1-F and 1-G.

Example 30-E: (300 mg, 8%). MS(ESI) m/z 407.2 (M+H)⁺. ¹H NMR (600 MHz, Chloroform-d) δ 7.64 (s, 1H), 3.93-3.85 (m, 1H), 3.52-3.49 (m, 2H), 3.26 (d, J=16.6 Hz, 1H), 2.95 (d, J=16.6 Hz, 1H), 2.41-2.17 (m, 2H), 2.16-1.84 (m, 7H), 1.75-1.72 (m, 1H), 1.57-1.54 (m, 2H), 1.52 (s, 9H). Compound 30-F: (3.2 g, 80%). MS(ESI) m/z 407.2 (M+H)⁺. ¹H NMR (600 MHz, Chloroform-d) δ 8.81 (s, 1H), 6.56 (s, 1H), 5.06-5.03 (m, 1H), 3.87-3.72 (m, 2H), 2.41-2.38 (m, 2H), 2.19-1.99 (m, 5H), 1.79-1.63 (m, 5H), 1.42-1.27 (m, 1H), 1.19 (s, 9H)

### Step 3: Synthesis of 7′-cyclopentyl-2′-((5-(piperazin-1-yl)pyridin-2-yl)amino)-5′,7′-dihydrospiro[piperidine-3,6′-pyrrolo[2,3-d]pyrimidin]-2-one (Compound C35)

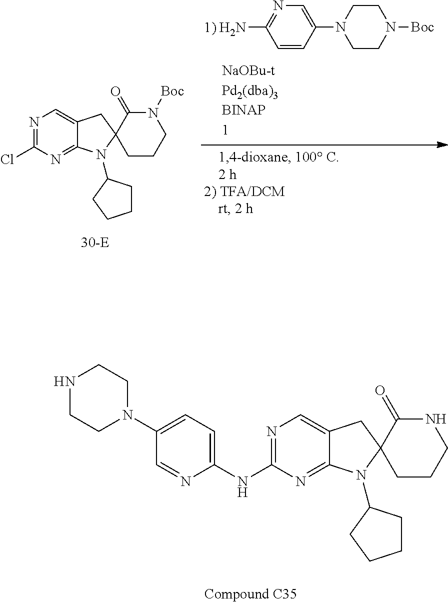

Compound C35 was synthesized following the general procedure A, 4 mg (7% yield). MS(ESI) m/z 449.3 (M+H)⁺.

### Example 31: Compound C36

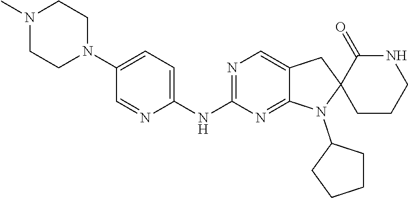 [SMILES: CN1CCN(c2ccc(Nc3ncc4c(n3)N(C3CCCC3)C3(CCCNC3=O)C4)nc2)CC1]

### Synthesis of 7′-cyclopentyl-2′-((5-(4-methylpiperazin-1-yl)pyridin-2-yl)amino)-5′,7′-dihydrospiro[piperidine-3,6′-pyrrolo[2,3-d]pyrimidin]-2-one (Compound C36)

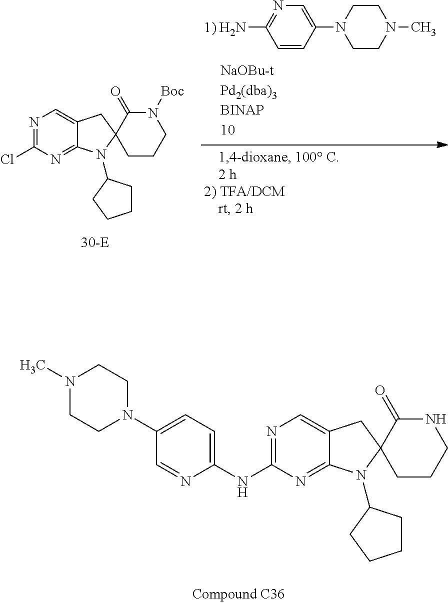

Compound C36 was synthesized following the general procedure A, 2 mg (4% yield). MS(ESI) m/z 463.3 (M+H)⁺.

### Example 32: Compound C37

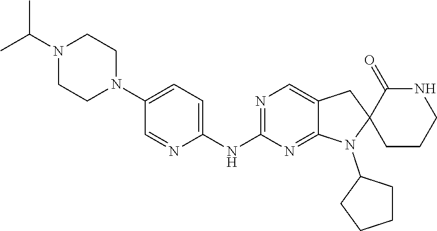 [SMILES: CC(C)N1CCN(c2ccc(Nc3ncc4c(n3)N(C3CCCC3)C3(CCCNC3=O)C4)nc2)CC1]

### Synthesis of 7′-cyclopentyl-2′-((5-(4-isopropylpiperazin-1-yl)pyridin-2-yl)amino)-5′,7′-dihydrospiro[piperidine-3,6′-pyrrolo[2,3-d]pyrimidin]-2-one (Compound C37)

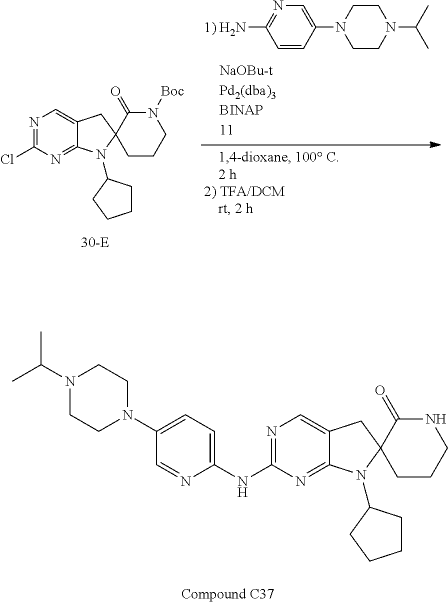

Compound C37 was synthesized following the general procedure A, 1 mg (2% yield). MS(ESI) m/z 491.3 (M+H)⁺.

### Example 33: Compound C74

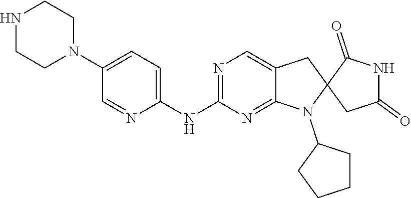 [SMILES: O=C1CC2(Cc3cnc(Nc4ccc(N5CCNCC5)cn4)nc3N2C2CCCC2)C(=O)N1]

### Step 1: Synthesis of tert-butyl (2-(phenylselanyl) acetyl)carbamate 33-C

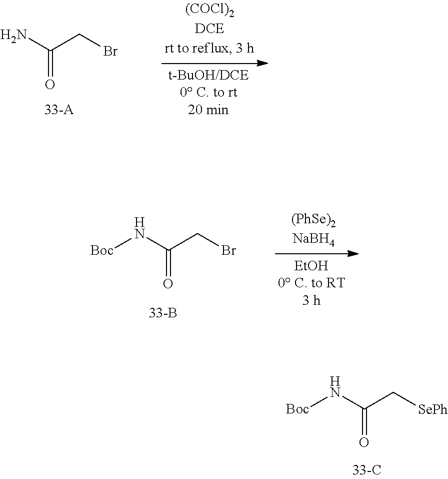

Synthesis of 33-B: (Ref: WO 2009096435): To the suspension of 2-bromoacetamide 33-A (15 g, 108.6 mmol) and 1,2-dichloroethane (75 mL), oxalyl chloride (11.5 mL, 321 mmol) was added dropwise at 0° C. The reaction mixture was heated under reflux for 3 hours, cooled to 0° C., tert-butyl alcohol/1,2-dichloroethane (30 mL, 1/1) was added and stirred for 20 minutes. To the reaction solution, saturated aqueous sodium bicarbonate solution was added and extracted with 1,2-dichloroethane. After washing with saturated aqueous sodium bicarbonate solution and water, the solution was dried over anhydrous sodium sulfate and filtered. The solvent was evaporated under reduced pressure to obtain 33-B (15 g, 58%) which was used without further purification for the next step. ¹H NMR (600 MHz, Chloroform-d) δ 7.70 (s, 1H), 4.30 (s, 2H), 1.51 (s, 9H).

Synthesis of 33-C: Prepared using the procedure described for the synthesis of compound 1-B. (5.5 g, 49% yield). MS(ESI) m/z 338.0 (M+Na)+. ¹H NMR (600 MHz, Chloroform-d) δ 7.67-7.51 (m, 3H), 7.29-7.25 (m, 2H), 3.93 (s, 1H), 2.39 (s, 2H), 1.49 (s, 9H).

### Step 2: Synthesis of tert-butyl 2′-chloro-7′-cyclopentyl-2,5-dioxo-5′,7′-dihydrospiro[pyrrolidine-3,6′-pyrrolo[2,3-d]pyrimidine]-1-carboxylate (33-F)

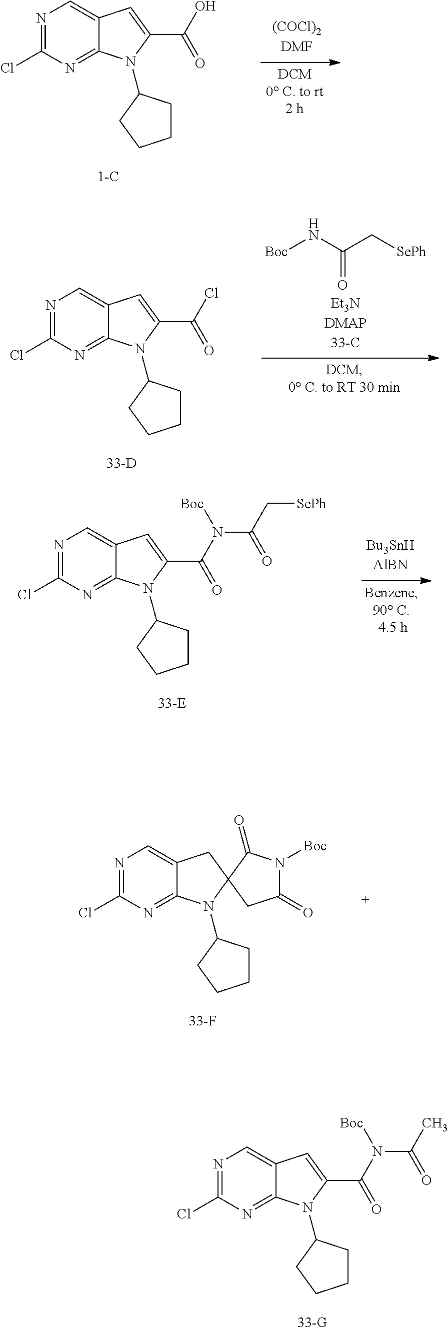

Synthesis of 33-D: (Ref: Kyei et al., Tetrahedron Lett. 45(48): 8931-8934 (2004)): To a cold (0° C.) solution of acid 1-C (8 g, 30.1 mmol) in dichloromethane (100 mL) was added DMF (0.5 mL) followed by dropwise addition of oxalyl chloride (10.3 mL, 120.4 mmol). The mixture was allowed to warm to room temperature and stirred for a period of 2 hours. The reaction mixture is concentrated in vacuo and is used for the next step without further purification.

Synthesis of 33-E: (Ref: Meng and Szostak, Org. Lett. 18(4): 796-799 (2016)): An oven-dried round-bottomed flask equipped with a stir bar was charged with 33-C (1 g, 3.4 mmol), triethylamine (1 mL, 7.5 mmol), 4-dimethylaminopyridine (115 mg, 25 mole percent) and dichloromethane (15 mL) was placed under a positive pressure of argon and subjected to three evacuation/backfilling cycles under high vacuum. Compound 33-D (1 g, 3.7 mmol) in 15 mL DCM was added dropwise to the reaction mixture with vigorous stirring at 0° C., and the reaction mixture was stirred at same temperature for 30 min. After the indicated time, the reaction mixture was diluted with DCM (50 mL) and was washed with 1 N HCl, brine, dried over sodium sulfate and concentrated. The crude product was purified by column chromatography (hexane:ethyl acetate, 4:1) to give 33-E (800 mg, 42%). MS(ESI) m/z 563.1 (M+H)⁺. ¹H NMR (600 MHz, Chloroform-d) δ 8.83 (s, 1H), 7.60-7.56 (m, 2H), 7.29 (s, 1H), 7.29-7.27 (m, 3H), 5.70-5.60 (m, 1H), 4.12 (s, 2H), 2.42-2.29 (m, 2H), 2.12 (s, 4H), 1.75-1.72 (m, 2H), 1.41 (s, 9H).

Synthesis of 33-F and 33-G: Prepared using the procedure described for the synthesis of compounds 1-F and 1-G.

Compound 33-F: (210 mg, 36%). MS(ESI) m/z 407.1 (M+H)⁺. ¹H NMR (600 MHz, Chloroform-d) δ 7.78 (s, 1H), 3.57-3.46 (m, 2H), 3.17-3.05 (m, 2H), 2.94 (d, J=18.6 Hz, 1H), 2.24-2.15 (m, 2H), 2.00-1.90 (m, 2H), 1.89-1.76 (m, 2H), 1.60 (s, 9H), 1.59-1.52 (m, 2H). Compound 33-G: (60 mg, 10%). MS(ESI) m/z 407.1 (M+H)⁺. ¹H NMR (600 MHz, Chloroform-d) δ 8.93 (s, 1H), 7.15 (s, 1H), 5.68-5.56 (m, 1H), 2.58 (s, 3H), 2.43-2.32 (m, 2H), 2.16-2.05 (m, 5H), 1.79-1.69 (m, 2H), 1.39 (s, 9H).

### Step 3: Synthesis of 7′-cyclopentyl-2′-((5-(piperazin-1-yl)pyridin-2-yl)amino)-5′,7′-dihydrospiro[pyrrolidine-3,6′-pyrrolo[2,3-d]pyrimidine]-2,5-dione (Compound C74)

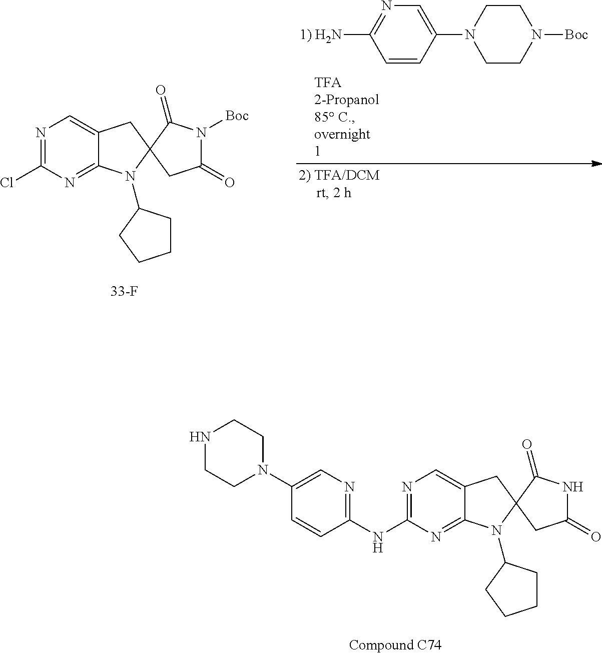

Compound C74 was synthesized following the general procedure C, 2 mg (6% yield). MS(ESI) m/z 449.3 (M+H)⁺. ¹H NMR (600 MHz, Chloroform-d) δ 7.69 (s, 1H), 7.50 (s, 2H), 6.66 (s, 1H), 3.95-3.88 (m, 4H), 3.50-3.41 (m, 2H), 3.07-3.03 (m, 4H), 3.00 (d, J=16.1 Hz, 1H), 2.91 (d, J=18.7 Hz, 1H), 2.38-2.26 (m, 3H), 1.94-1.76 (m, 6H).

### Example 34: Compound C75

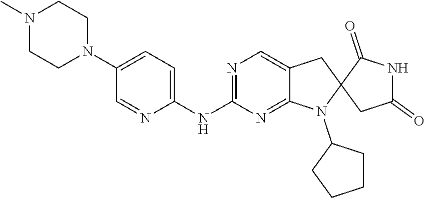 [SMILES: CN1CCN(c2ccc(Nc3ncc4c(n3)N(C3CCCC3)C3(CC(=O)NC3=O)C4)nc2)CC1]

### Synthesis of 7′-cyclopentyl-2′-((5-(4-methylpiperazin-1-yl)pyridin-2-yl)amino)-5′,7′-dihydrospiro[pyrrolidine-3,6′-pyrrolo[2,3-d]pyrimidine]-2,5-dione (Compound C75)

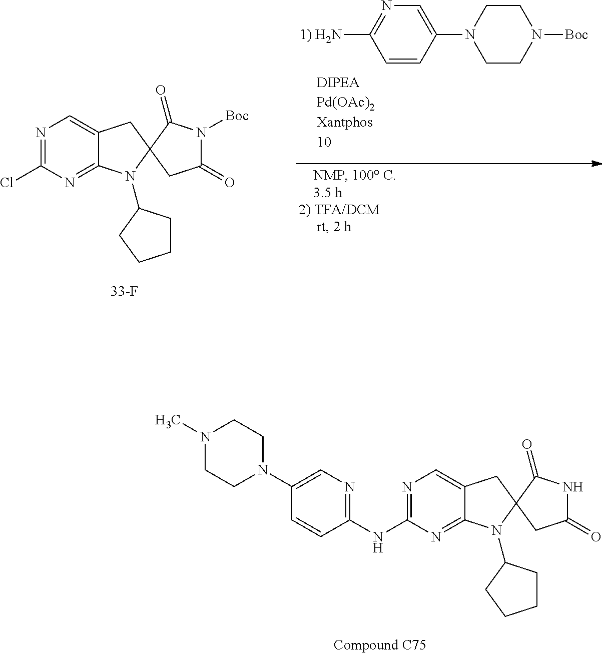

Compound C75 was synthesized following the general procedure I, 2 mg (4% yield). MS(ESI) m/z 463.3 (M+H)⁺. ¹H NMR (600 MHz, Chloroform-d) δ 8.59 (s, 1H), 8.17 (d, J=9.0 Hz, 1H), 8.03 (d, J=2.7 Hz, 1H), 7.62 (s, 1H), 7.34 (dd, J=9.1, 2.9 Hz, 1H), 6.68 (s, 1H), 5.99-5.89 (m, 1H), 5.31 (s, 1H), 3.51 (s, 2H), 3.24 (s, 4H), 2.70 (s, 4H), 2.43 (s, 3H), 2.40-2.31 (m, 3H), 2.07 (s, 3H), 1.95-1.86 (m, 3H).

### Example 35 Compound C76

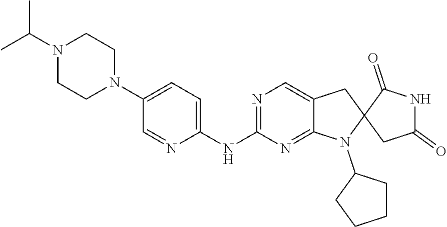 [SMILES: CC(C)N1CCN(c2ccc(Nc3ncc4c(n3)N(C3CCCC3)C3(CC(=O)NC3=O)C4)nc2)CC1]

### Synthesis of 7′-cyclopentyl-2′-((5-(4-isopropylpiperazin-1-yl)pyridin-2-yl)amino)-5′,7′-dihydrospiro[pyrrolidine-3,6′-pyrrolo[2,3-d]pyrimidine]-2,5-dione (Compound C76)

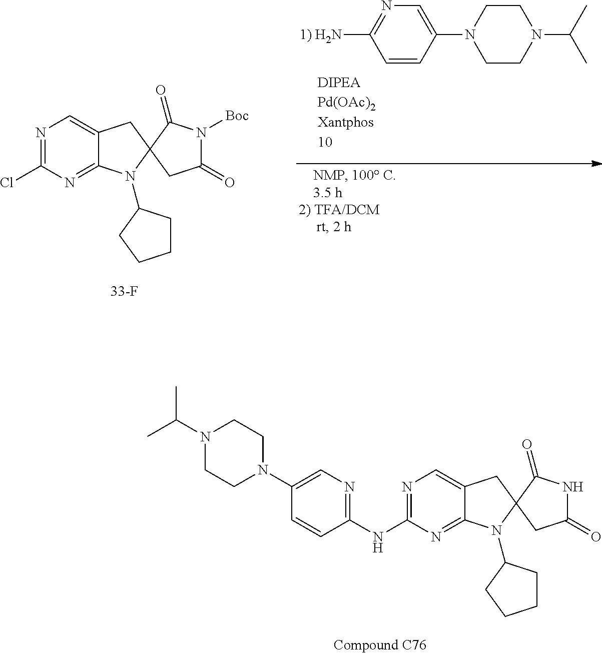

Compound C76 was synthesized following the general procedure I, 2 mg (4% yield). MS(ESI) m/z 491.3 (M+H)⁺.

### Example 36: Compound C77

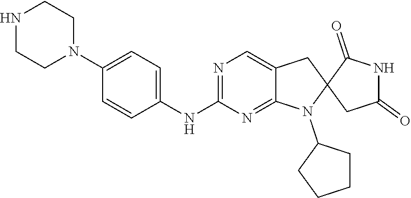 [SMILES: O=C1CC2(Cc3cnc(Nc4ccc(N5CCNCC5)cc4)nc3N2C2CCCC2)C(=O)N1]

### Synthesis of 7′-cyclopentyl-2′-((4-(piperazin-1-yl)phenyl)amino)-5′,7′-dihydrospiro[pyrrolidine-3,6′-pyrrolo[2,3-d]pyrimidine]-2,5-dione (Compound-C77)

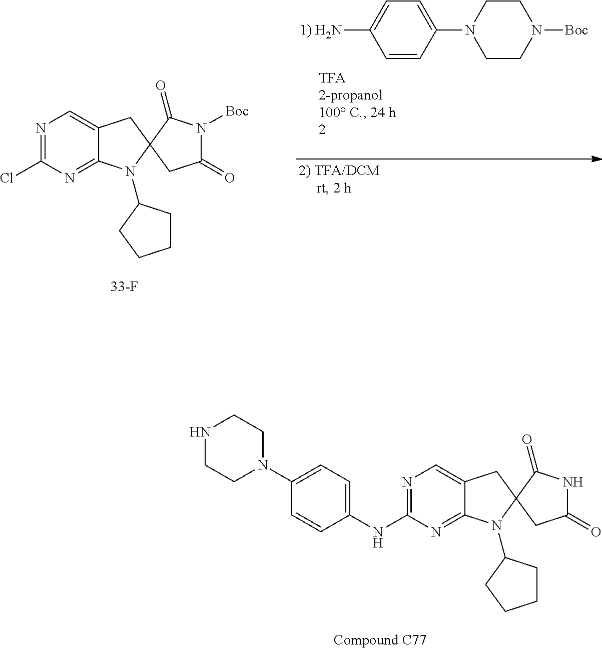

Compound C77 was synthesized following the general procedure C, 2 mg (6% yield). MS(ESI) m/z 448.3 (M+H)⁺.

### Example 37: Compound C78

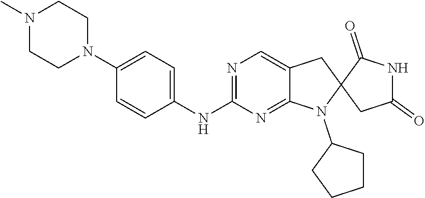 [SMILES: CN1CCN(c2ccc(Nc3ncc4c(n3)N(C3CCCC3)C3(CC(=O)NC3=O)C4)cc2)CC1]

### Synthesis of 7-cyclopentyl-2′-((4(4-methylpiperazin-1-yl)phenyl)amino)-5′,7′-dihydrospiro[pyrrolidine-3,6′-pyrrolo[2,3-d]pyrimidine]-2,5-dione (Compound C78)

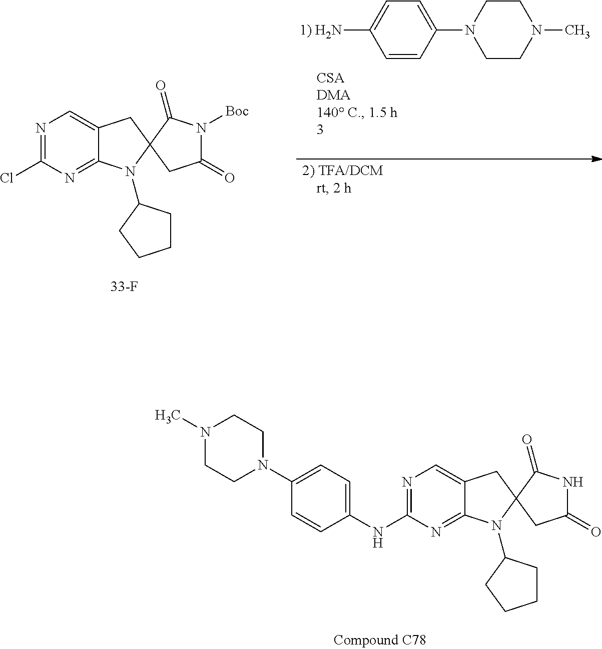

Compound C78 was synthesized following the general procedure G, 3 mg (9% yield). MS(ESI) m/z 462.3 (M+H)⁺.

### Example 38: Compound C79

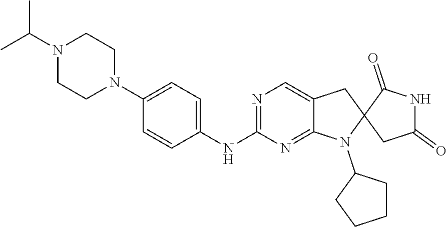 [SMILES: CC(C)N1CCN(c2ccc(Nc3ncc4c(n3)N(C3CCCC3)C3(CC(=O)NC3=O)C4)cc2)CC1]

### Synthesis of 7′-cyclopentyl-2′-((4-(4-isopropylpiperazin-1-yl)phenyl)amino)-5′,7′-dihydrospiro[pyrrolidine-3,6′-pyrrolo[2,3-d]pyrimidine]-2,5-dione (Compound C79)

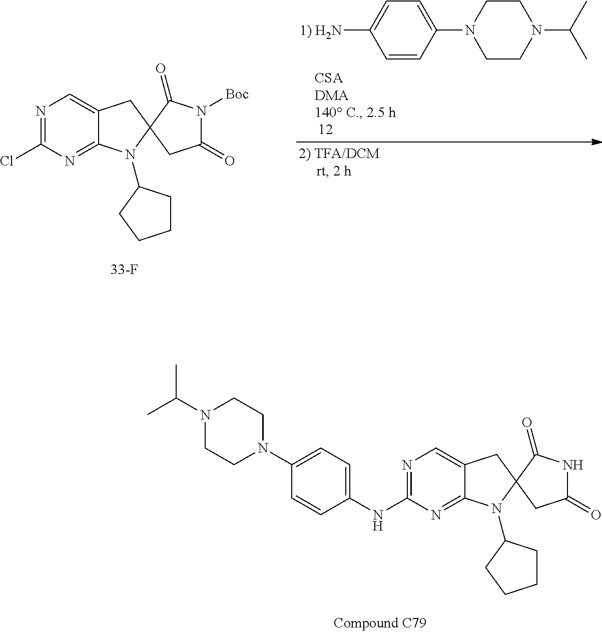

Compound C79 was synthesized following the general procedure G, 3 mg (5% yield). MS(ESI) m/z 490.3 (M+H)⁺.

### Example 39: Compound C80

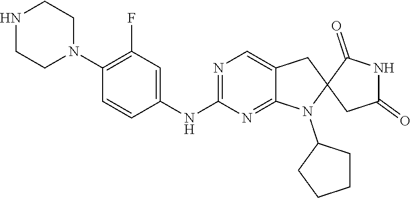 [SMILES: O=C1CC2(Cc3cnc(Nc4ccc(N5CCNCC5)c(F)c4)nc3N2C2CCCC2)C(=O)N1]

### Synthesis of 7′-cyclopentyl-2′-((3-fluoro-4-(piperazin-1-yl)phenyl)amino)-5′,7′-dihydrospiro[pyrrolidine-3,6′-pyrrolo[2,3-d]pyrimidine]-2,5-dione (Compound C80)

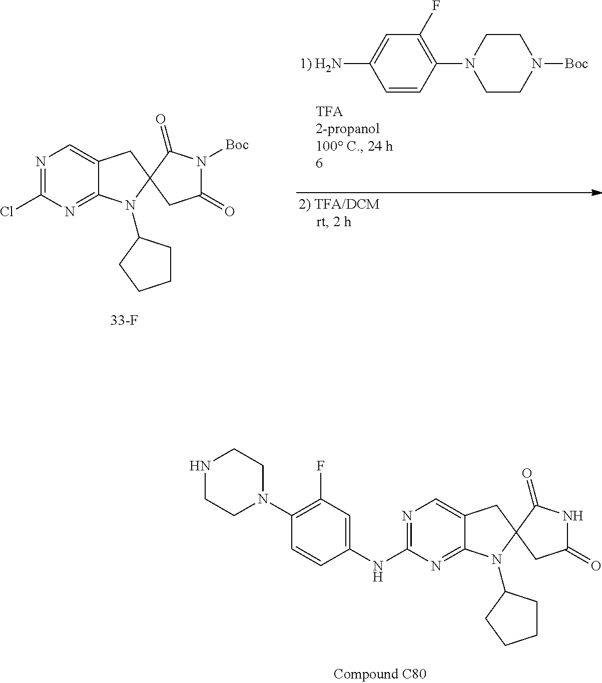

Compound C80 was synthesized following the general procedure C, 6 mg (17% yield). MS(ESI) m/z 466.3 (M+H)⁺.

### Example 40: Compound C81

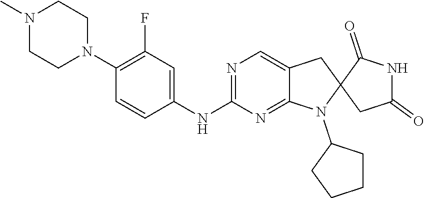 [SMILES: CN1CCN(c2ccc(Nc3ncc4c(n3)N(C3CCCC3)C3(CC(=O)NC3=O)C4)cc2F)CC1]

### Synthesis of 7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidine]-2,5-dione (Compound C81)

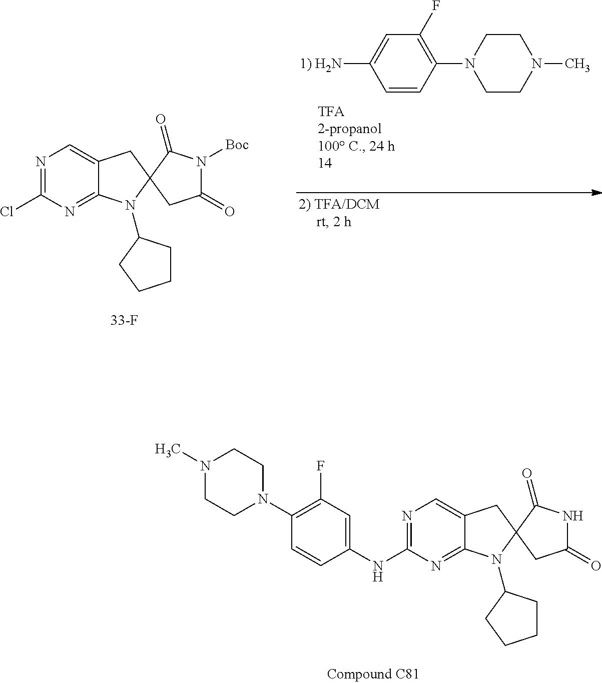

Compound C81 was synthesized following the general procedure C, 4 mg (8% yield). MS(ESI) m/z 480.3 (M+H)⁺. ¹H NMR (600 MHz, Chloroform-d) δ 7.76 (s, 1H), 7.69-7.57 (m, 2H), 7.07-6.99 (m, 1H), 6.89 (t, J=9.1 Hz, 1H), 3.48 (d, J=16.7 Hz, 1H), 3.45-3.39 (m, 1H), 3.10 (s, 1H), 3.08 (s, 2H), 3.07 (s, 2H), 3.02 (d, J=16.1 Hz, 1H), 2.92 (d, J=18.7 Hz, 1H), 2.64 (s, 4H), 2.44-2.38 (m, 2H), 2.37 (s, 3H), 1.97-1.88 (m, 2H), 1.85-1.75 (m, 2H), 1.61-1.48 (m, 2H).

### Example 41: Compound C82

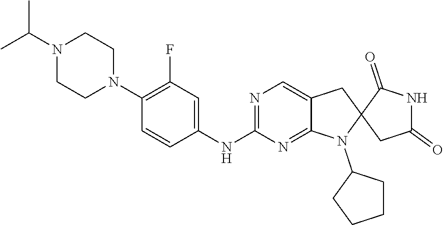 [SMILES: CC(C)N1CCN(c2ccc(Nc3ncc4c(n3)N(C3CCCC3)C3(CC(=O)NC3=O)C4)cc2F)CC1]

### Synthesis of 7′-cyclopentyl-2′-((3-fluoro-4-(4-isopropylpiperazin-1-yl)phenyl)amino)-5′,7′-dihydrospiro[pyrrolidine-3,6′-pyrrolo[2,3-d]pyrimidine]-2,5-dione (Compound C82)

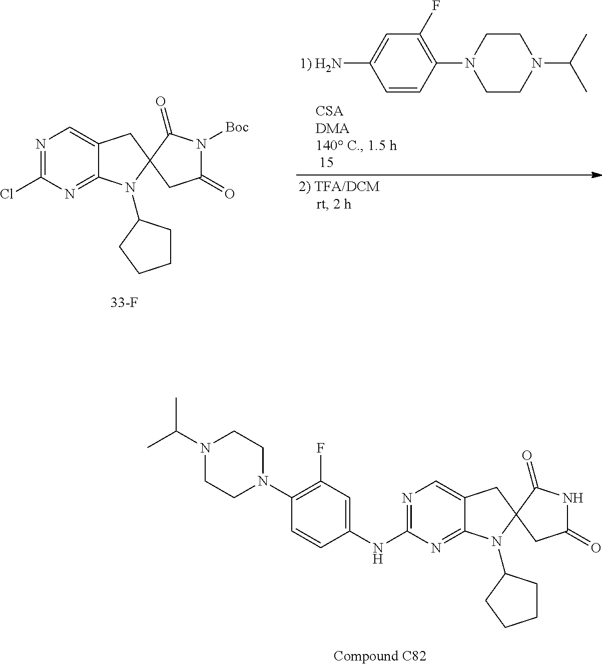

Compound C82 was synthesized following the general procedure G, 3 mg (5% yield). MS(ESI) m/z 508.3 (M+H)⁺. ¹H NMR (600 MHz, Chloroform-d) δ 7.66 (s, 1H), 7.63 (dd, J=14.8, 2.1 Hz, 1H), 7.12 (s, 1H), 6.99 (d, J=6.9 Hz, 1H), 6.89 (t, J=9.1 Hz, 1H), 3.47 (d, J=16.5 Hz, 1H), 3.45-3.39 (m, 1H), 3.12-3.10 (m, 1H), 3.10-3.06 (m, 4H), 3.02 (d, J=15.8 Hz, 1H), 2.92 (d, J=18.8 Hz, 1H), 2.73 (s, 5H), 2.44-2.32 (m, 3H), 1.92 (s, 2H), 1.86-1.74 (m, 3H), 1.10 (d, J=6.5 Hz, 6H).

### Example 42: Compound C83

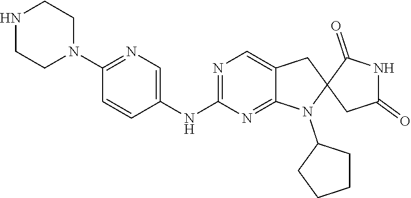 [SMILES: O=C1CC2(Cc3cnc(Nc4ccc(N5CCNCC5)nc4)nc3N2C2CCCC2)C(=O)N1]

### Synthesis of 7′-cyclopentyl-2′-((6-(piperazin-1-yl)pyridin-3-yl)amino)-5′,7′-dihydrospiro[pyrrolidine-3,6′-pyrrolo[2,3-d]pyrimidine]-2,5-dione (Compound C83)

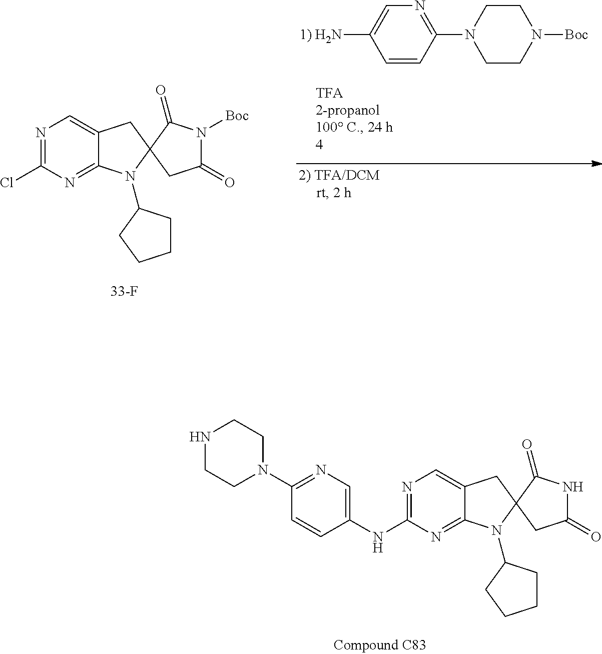

Compound C83 was synthesized following the general procedure C, 3 mg (9% yield). MS(ESI) m/z 449.2 (M+H)⁺.

### Example 43: Compound C84

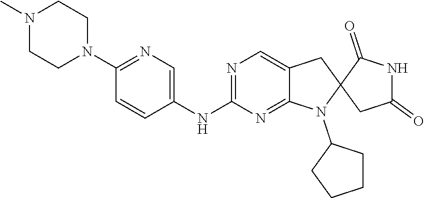 [SMILES: CN1CCN(c2ccc(Nc3ncc4c(n3)N(C3CCCC3)C3(CC(=O)NC3=O)C4)cn2)CC1]

### Synthesis of 7′-cyclopentyl-2′-((6-(4-methylpiperazin-1-yl)pyridin-3-yl)amino)-5′,7′-dihydrospiro[pyrrolidine-3,6′-pyrrolo[2,3-d]pyrimidine]-2,5-dione (Compound C84)

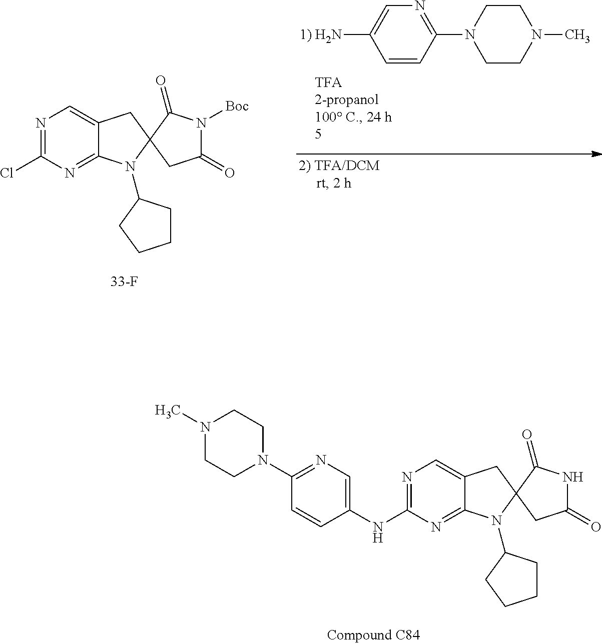

Compound C84 was synthesized following the general procedure C, 5 mg (15% yield). MS(ESI) m/z 463.3 (M+H)⁺. ¹H NMR (600 MHz, Chloroform-d) δ 8.27 (d, J=2.4 Hz, 1H), 7.68 (d, J=7.1 Hz, 1H), 7.51 (s, 1H), 6.64 (d, J=9.0 Hz, 1H), 3.57 (s, 4H), 3.47 (dd, J=16.0, 1.2 Hz, 1H), 3.44-3.36 (m, 1H), 3.08 (d, J=18.7 Hz, 1H), 3.02 (d, J=16.8 Hz, 1H), 2.92 (d, J=18.8 Hz, 1H), 2.65 (s, 3H), 2.42 (s, 4H), 2.29-2.19 (m, 3H), 1.80-1.69 (m, 2H), 1.52-1.41 (m, 3H).

### Example 44: Compound C85

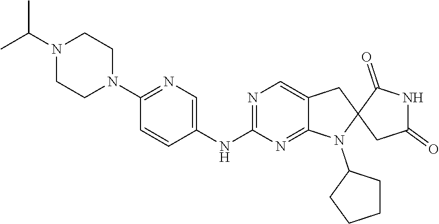 [SMILES: CC(C)N1CCN(c2ccc(Nc3ncc4c(n3)N(C3CCCC3)C3(CC(=O)NC3=O)C4)cn2)CC1]

### Synthesis of 7′-cyclopentyl-2′-((6-(4-isopropylpiperazin-1-yl)pyridin-3-yl)amino)-5′,7′-dihydrospiro[pyrrolidine-3,6′-pyrrolo[2,3-d]pyrimidine]-2,5-dione (Compound C85)

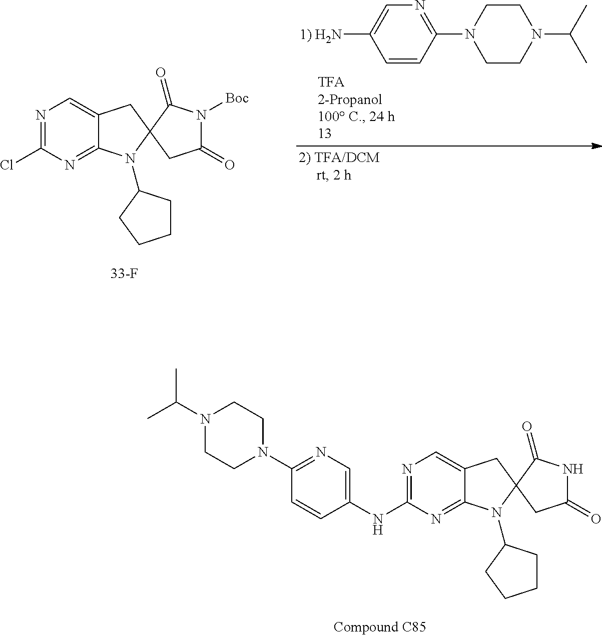

Compound C85 was synthesized following the general procedure C, 3 mg (8% yield). MS(ESI) m/z 491.3 (M+H)⁺.

### Example 45: Compound C86

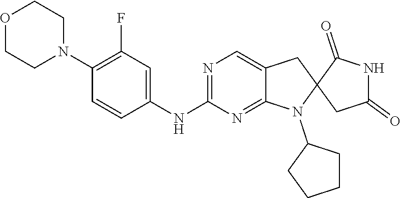 [SMILES: O=C1CC2(Cc3cnc(Nc4ccc(N5CCOCC5)c(F)c4)nc3N2C2CCCC2)C(=O)N1]

### Synthesis of 7′-cyclopentyl-2′-((3-fluoro-4-morpholinophenyl)amino)-5′,7′-dihydrospiro[pyrrolidine-3,6′-pyrrolo[2,3-d]pyrimidine]-2,5-dione (Compound C86)

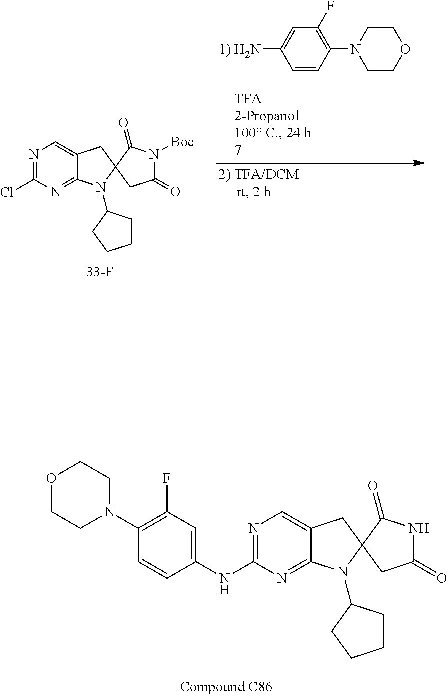

Compound C86 was synthesized following the general procedure C, 14 mg (41% yield). MS(ESI) m/z 467.2 (M+H)⁺. ¹H NMR (600 MHz, Chloroform-d) δ 7.91 (s, 1H), 7.67 (dd, J=14.8, 2.4 Hz, 1H), 7.63 (s, 1H), 7.07-6.99 (m, 1H), 6.87 (t, J=9.1 Hz, 1H), 3.90-3.83 (m, 4H), 3.48 (dd, J=15.8, 1.1 Hz, 1H), 3.46-3.39 (m, 1H), 3.10 (d, J=18.7 Hz, 1H), 3.06-3.00 (m, 5H), 2.93 (d, J=18.7 Hz, 1H), 2.45-2.34 (m, 2H), 2.00-1.89 (m, 2H), 1.89-1.61 (m, 2H), 1.61-1.48 (m, 2H).

### Example 46: Compound C87

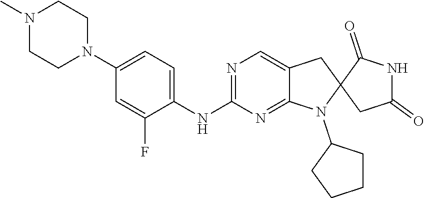 [SMILES: CN1CCN(c2ccc(Nc3ncc4c(n3)N(C3CCCC3)C3(CC(=O)NC3=O)C4)c(F)c2)CC1]

### Synthesis of 7′-cyclopentyl-2′-((2-fluoro-4-(4-methylpiperazin-1-yl)phenyl)amino)-5′,7′-dihydrospiro[pyrrolidine-3,6′-pyrrolo[2,3-d]pyrimidine]-2,5-dione (Compound C87)

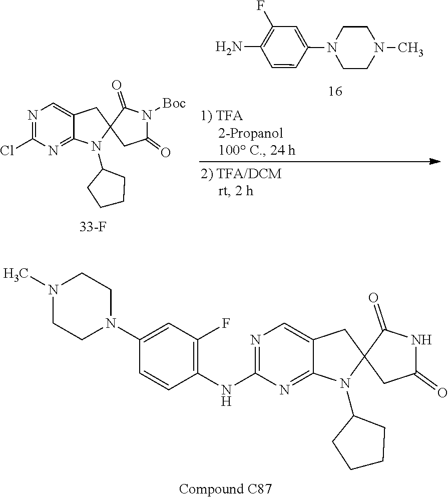 [SMILES: CN1CCN(c2ccc(N)c(F)c2)CC1 ; CC(C)(C)OC(=O)N1C(=O)CC2(Cc3cnc(Cl)nc3N2C2CCCC2)C1=O ; CN1CCN(c2ccc(Nc3ncc4c(n3)N(C3CCCC3)C3(CC(=O)NC3=O)C4)c(F)c2)CC1]

Compound C87 was synthesized following the general procedure C, 3 mg (8% yield). MS(ESI) m/z 480.2 (M+H)⁺.

### Example 47: Compound C88

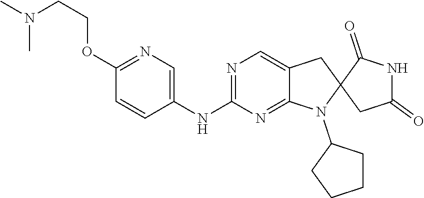 [SMILES: CN(C)CCOc1ccc(Nc2ncc3c(n2)N(C2CCCC2)C2(CC(=O)NC2=O)C3)cn1]

### Synthesis of 7′-cyclopentyl-2′-((6-(2-(dimethylamino)ethoxy)pyridin-3-yl)amino)-5′,7′-dihydrospiro[pyrrolidine-3,6′-pyrrolo[2,3-d]pyrimidine]-2,5-dione (Compound C88)

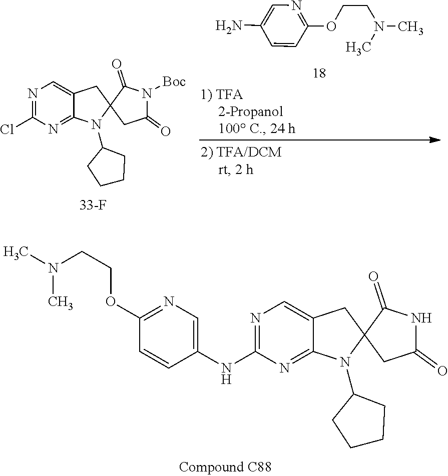 [SMILES: CN(C)CCOc1ccc(N)cn1 ; CC(C)(C)OC(=O)N1C(=O)CC2(Cc3cnc(Cl)nc3N2C2CCCC2)C1=O ; CN(C)CCOc1ccc(Nc2ncc3c(n2)N(C2CCCC2)C2(CC(=O)NC2=O)C3)cn1]

Compound C88 was synthesized following the general procedure C, 15 mg (45% yield). MS(ESI) m/z 452.2 (M+H)⁺.

### Example 48: Compound C89

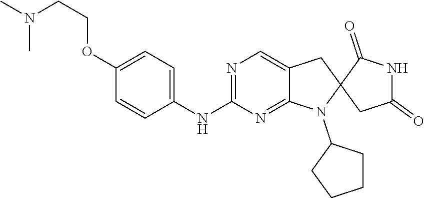 [SMILES: CN(C)CCOc1ccc(Nc2ncc3c(n2)N(C2CCCC2)C2(CC(=O)NC2=O)C3)cc1]

### Synthesis of 7′-cyclopentyl-2′-((4-(2-(dimethylamino)ethoxy)phenyl)amino)-5′,7′-dihydrospiro[pyrrolidine-3,6′-pyrrolo[2,3-d]pyrimidine]-2,5-dione (Compound C89)

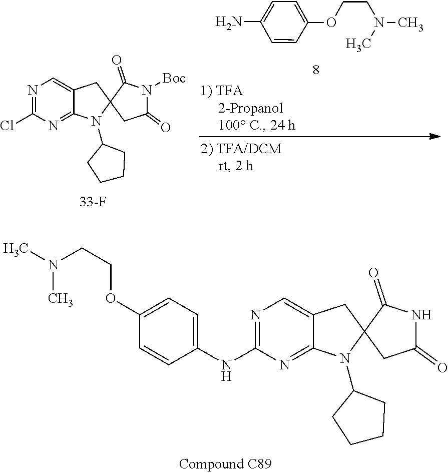 [SMILES: CN(C)CCOc1ccc(N)cc1 ; CC(C)(C)OC(=O)N1C(=O)CC2(Cc3cnc(Cl)nc3N2C2CCCC2)C1=O ; CN(C)CCOc1ccc(Nc2ncc3c(n2)N(C2CCCC2)C2(CC(=O)NC2=O)C3)cc1]

Compound C89 was synthesized following the general procedure C, 8 mg (24% yield). MS(ESI) m/z 451.2 (M+H)⁺.

### Example 49: Compound C90

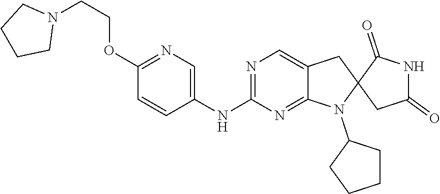 [SMILES: O=C1CC2(Cc3cnc(Nc4ccc(OCCN5CCCC5)nc4)nc3N2C2CCCC2)C(=O)N1]

### Synthesis of 7′-cyclopentyl-2′-((6-(2-(pyrrolidin-1-yl)ethoxy)pyridin-3-yl)amino)-5′,7′-dihydrospiro[pyrrolidine-3,6′-pyrrolo[2,3-d]pyrimidine]-2,5-dione (Compound C90)

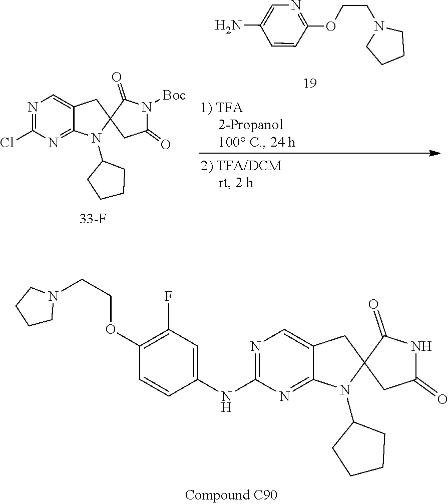 [SMILES: Nc1ccc(OCCN2CCCC2)nc1 ; CC(C)(C)OC(=O)N1C(=O)CC2(Cc3cnc(Cl)nc3N2C2CCCC2)C1=O ; O=C1CC2(Cc3cnc(Nc4ccc(OCCN5CCCC5)c(F)c4)nc3N2C2CCCC2)C(=O)N1]

Compound C90 was synthesized following the general procedure C, 8 mg (23% yield). MS(ESI) m/z 478.3 (M+H)⁺.

### Example 50: Compound C91

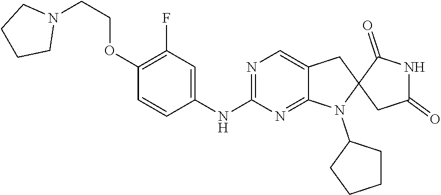 [SMILES: O=C1CC2(Cc3cnc(Nc4ccc(OCCN5CCCC5)c(F)c4)nc3N2C2CCCC2)C(=O)N1]

### Synthesis of 7′-cyclopentyl-2′-((3-fluoro-4-(2-(pyrrolidin-1-yl)ethoxy)phenyl)amino)-5′,7′-dihydrospiro[pyrrolidine-3,6′-pyrrolo[2,3-d]pyrimidine]-2,5-dione (Compound C91)

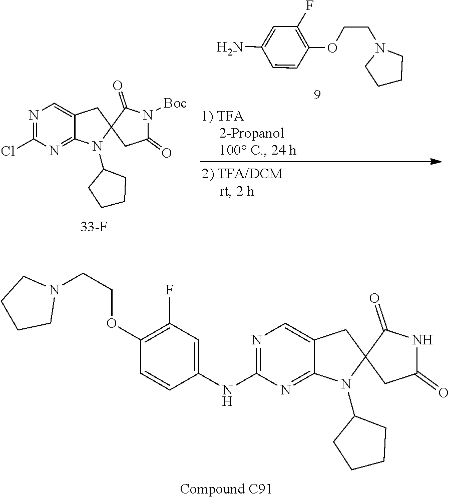 [SMILES: CC(C)(C)OC(=O)N1C(=O)CC2(Cc3cnc(Cl)nc3N2C2CCCC2)C1=O ; Nc1ccc(OCCN2CCCC2)c(F)c1 ; O=C1CC2(Cc3cnc(Nc4ccc(OCCN5CCCC5)c(F)c4)nc3N2C2CCCC2)C(=O)N1]

Compound C91 was synthesized following the general procedure C, 6 mg (16% yield). MS(ESI) m/z 495.3 (M+H)⁺. ¹H NMR (600 MHz, Chloroform-d) δ 7.56 (dd, J=12.9, 2.2 Hz, 1H), 7.46 (s, 1H), 7.16 (d, J=8.7 Hz, 1H), 6.93 (t, J=9.0 Hz, 1H), 4.43-4.35 (m, 2H), 3.55-3.39 (m, 5H), 3.09 (dd, J=36.5, 18.0 Hz, 2H), 2.96 (d, J=18.8 Hz, 2H), 2.35-2.24 (m, 3H), 2.14-2.07 (m, 6H), 1.89-1.78 (m, 3H), 1.61-1.48 (m, 2H).

### Example 51: Compound C92

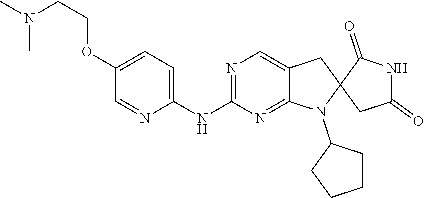 [SMILES: CN(C)CCOc1ccc(Nc2ncc3c(n2)N(C2CCCC2)C2(CC(=O)NC2=O)C3)nc1]

### Synthesis of 7′-cyclopentyl-2′-((5-(2-(dimethylamino)ethoxy)pyridin-2-yl)amino)-5′,7′-dihydrospiro[pyrrolidine-3,6′-pyrrolo[2,3-d]pyrimidine]-2,5-dione (Compound C92)

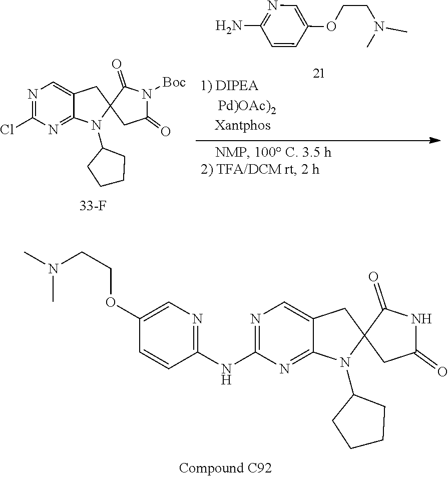 [SMILES: CN(C)CCOc1ccc(N)nc1 ; CC(C)(C)OC(=O)N1C(=O)CC2(Cc3cnc(Cl)nc3N2C2CCCC2)C1=O ; CN(C)CCOc1ccc(Nc2ncc3c(n2)N(C2CCCC2)C2(CC(=O)NC2=O)C3)nc1]

Compound C92 was synthesized following the general procedure I, 2 mg (4% yield). MS(ESI) m/z 452.2 (M+H)⁺. ¹H NMR (600 MHz, Chloroform-d) δ 8.59 (s, 1H), 8.25 (d, J=9.1 Hz, 1H), 8.04 (d, J=2.8 Hz, 1H), 7.63 (s, 1H), 7.34 (dd, J=9.1, 3.0 Hz, 1H), 6.66 (s, 1H), 6.00-5.87 (m, 1H), 5.28 (s, 1H), 4.27 (s, 2H), 3.51 (s, 2H), 3.10 (s, 2H), 2.63 (s, 6H), 2.40-2.30 (m, 2H), 1.98-1.85 (m, 3H), 1.82-1.70 (m, 6H).

### Example 52: Compound C93

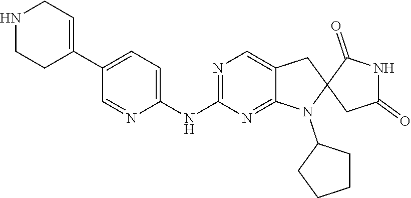 [SMILES: O=C1CC2(Cc3cnc(Nc4ccc(C5=CCNCC5)cn4)nc3N2C2CCCC2)C(=O)N1]

### Synthesis of 7′-cyclopentyl-2′-((1′,2′,3′,6′-tetrahydro-[3,4′-bipyridin]-6-yl)amino)-5′,7′-dihydrospiro[pyrrolidine-3,6′-pyrrolo[2,3-d]pyrimidine]-2,5-dione (Compound C93)

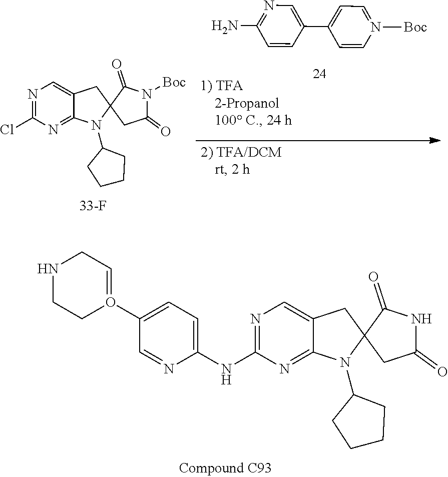

Compound C93 was synthesized following the general procedure C, 3 mg (9% yield). MS(ESI) m/z 446.2 (M+H)⁺.

### Example 53: Compound C94

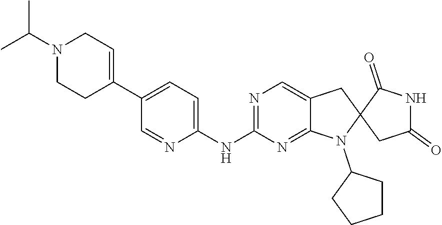 [SMILES: CC(C)N1CC=C(c2ccc(Nc3ncc4c(n3)N(C3CCCC3)C3(CC(=O)NC3=O)C4)nc2)CC1]

### Synthesis of 7′-cyclopentyl-2′-((1′-isopropyl-1′,2′,3′,6′-tetrahydro-[3,4′-bipyridin]-6-yl)amino)-5′,7′-dihydrospiro[pyrrolidine-3,6′-pyrrolo[2,3-d]pyrimidine]-2,5-dione (Compound C94)

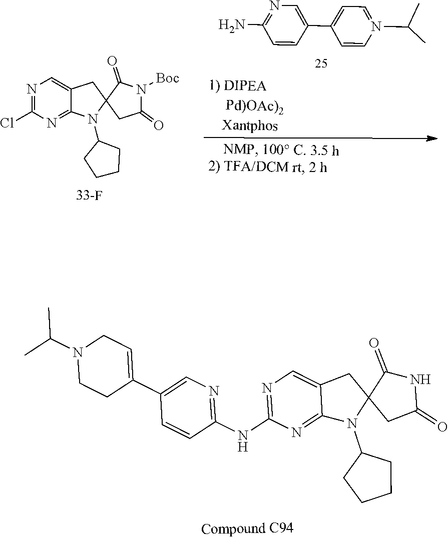

Compound C94 was synthesized following the general procedure I, 3 mg (6% yield). MS(ESI) m/z 488.3 (M+H)⁺.

### Example 54: Compound C19

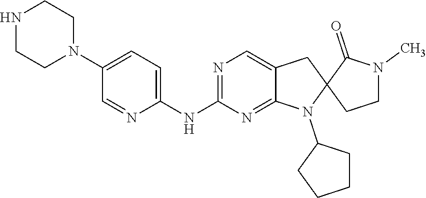 [SMILES: CN1CCC2(Cc3cnc(Nc4ccc(N5CCNCC5)cn4)nc3N2C2CCCC2)C1=O]

Step-1: Synthesis of N-methyl-2-(phenylselanyl)ethan-1-amine (54-C)

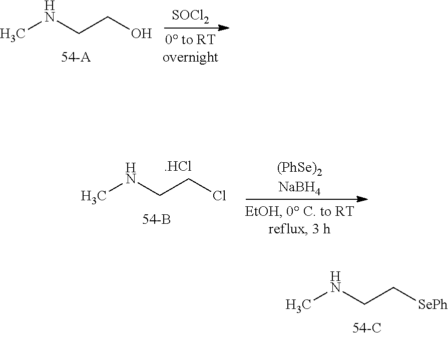

Synthesis of 54-B: (Ref: Shaikh et al., Organometallics 31(21): 7427-7433 (2012)): 54-A (23 g, 306 mmol) was dissolved in 200 mL CH₂Cl₂ and cooled to 0° C. SOCl₂ (28 mL in 25 mL CH₂Cl₂) was slowly added dropwise and the reaction mixture was stirred for overnight at RT The reaction mixture was evaporated to dryness on a rotary evaporator and the resulting crude product 54-B was used as such for next step (36 g, 90%). ¹H NMR (600 MHz, Chloroform-d) 3.99 (t, J=6.2 Hz, 2H), 3.35 (t, J=6.2 Hz, 3H), 2.81 (s, 3H).

Synthesis of 54-C: (Ref: Kyei et al., Tetrahedron Lett. 45(48): 8931-8934 (2004)): NaBH₄ (18.5 g, 500 mmol) was added portion wise to a stirred solution of diphenyldiselenide (35 g, 112 mmol) in absolute EtOH (150 mL) under argon at 0° C. After complete addition of NaBH₄, within 15 min the reaction medium became colorless. When the evolution of hydrogen ceased, a solution of 54-B (29 g, 223 mmol) in absolute EtOH (150 mL) was added dropwise at 0° C. The reaction mixture was then heated at reflux for 3 h. Upon completion of the reaction, brine (10 mL) was added and the solvent was evaporated under vacuum. The residue was dissolved in EtOAc (500 mL) and washed with saturated aqueous sodium hydrogen carbonate, water, and then brine. The organic layer was dried over Na₂SO₄, evaporated under vacuum and purified by silica gel column chromatography (DCM:MeOH, 98:2) to give 54-C (31 g, 65% yield). MS(ESI) m/z 216.1 (M+H)⁺. ¹HNMR: ¹H NMR (600 MHz, Chloroform-d) δ 7.51 (m, 2H), 7.32-7.20 (m, 3H), 3.05 (t, J=6.7 Hz, 2H), 2.85 (t, J=6.7 Hz, 2H), 2.42 (s, 3H), 2.18 (s, 1H).

### Step-2: Synthesis of 2′-chloro-7′-cyclopentyl-1-methyl-5′,7′-dihydrospiro[pyrrolidine-3,6′-pyrrolo[2,3-d]pyrimidin]-2-one (6)

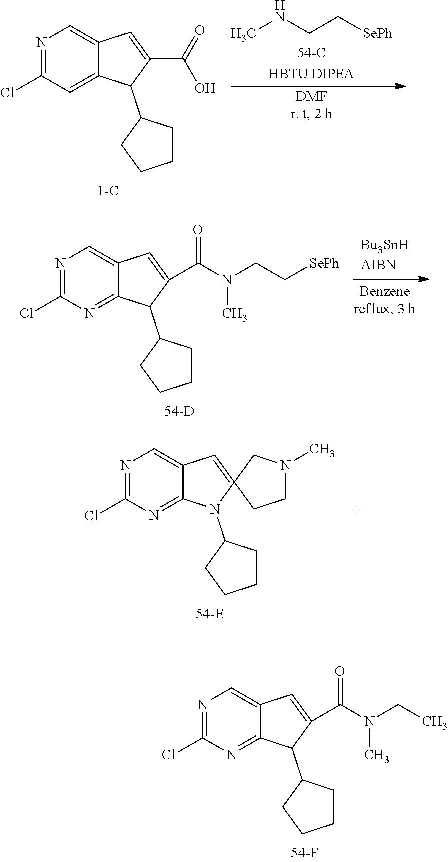

Synthesis of 54-D: Prepared according to the procedure described for 1-D (7.0 g, 81% yield). MS(ESI) m/z 463.1 [M+H]⁺. ¹H NMR (600 MHz, Chloroform-d) δ 8.79 (s, 0.5H), 8.67 (s, 0.5H), 7.58 (d, J=6.1 Hz, 1H), 7.31 (d, J=6.9 Hz, 1H), 7.23 (d, J=7.1 Hz, 1H), 7.01 (m, 2H), 6.31 (s, 1H), 4.94-4.91 (m, 0.5H), 4.79-4.65 (m, 0.5H), 3.85 (s, 1H), 3.64-3.53 (m, 1H), 3.23 (t, J=6.4 Hz, 1H), 3.09 (m, 3H), 2.95-2.86 (m, 1H), 2.44-2.25 (m, 2H), 2.07 (m, 4H), 1.67 (s, 2H).

Synthesis of 54-E and 54-F: Prepared according to the procedure described for 1-F and 1-G Compound 54-E: (1.9 g, 41% yield). MS(ESI) m/z 307.1 [M+H]⁺. ¹H NMR (600 MHz, Chloroform-d) δ 7.67 (s, 1H), 3.49-3.34 (m, 3H), 3.19 (dd, J=16.3, 1.6 Hz, 1H), 2.95 (s, 3H), 2.90-2.86 (m, 1H), 2.40-2.27 (m, 2H), 2.19 (m, 2H), 1.94 (m, 2H), 1.89-1.81 (m, 1H), 1.73 (m, 1H), 1.59-1.46 (m, 2H).

Compound 54-F: (1.2 g, 26% yield). MS(ESI) m/z 307.1 [M+H]⁺. ¹H NMR (600 MHz, Chloroform-d) δ 8.79 (s, 1H), 6.51 (s, 1H), 4.95-4.79 (m, 1H), 3.69-3.57 (m, 1H), 3.42-3.39 (m, 1H), 3.13-3.04 (m, 3H), 2.37 (s, 2H), 2.21-1.93 (m, 4H), 1.70-1.63 (m, 2H), 1.24 (m, 3H).

### Step-3: Synthesis of 7′-cyclopentyl-1-methyl-2′-((5-(piperazin-1-yl)pyridin-2-yl)amino)-5′,7′-dihydrospiro[pyrrolidine-3,6′-pyrrolo[2,3-d]pyrimidin]-2-one (Compound C19)

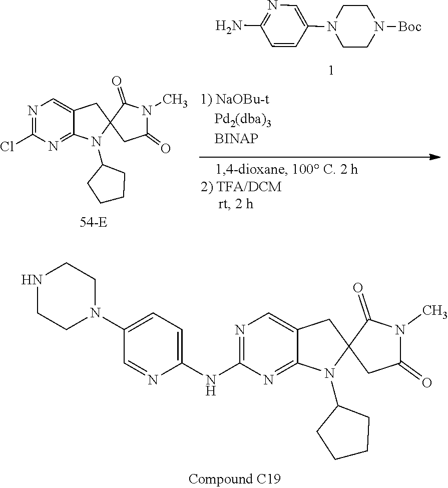 [SMILES: CC(C)(C)OC(=O)N1CCN(c2ccc(N)nc2)CC1 ; CN1C(=O)CC2(Cc3cnc(Cl)nc3N2C2CCCC2)C1=O ; CN1C(=O)CC2(Cc3cnc(Nc4ccc(N5CCNCC5)cn4)nc3N2C2CCCC2)C1=O]

Compound C19 was synthesized following the general procedure A, 33 mg (28% yield). MS(ESI) m/z 449.3 (M+H)⁺. ¹H NMR (600 MHz, DMSO-d₆) δ 8.90 (bs, 2H), 8.01 (s, 1H), 7.72 (s, 1H), 7.69 (dd, J=9.1, 2.7 Hz, 1H), 7.32 (s, 1H), 3.68-3.55 (m, 1H), 3.47-3.40 (m, 1H), 3.40-3.36 (m, 5H), 3.30-3.24 (m, 4H), 3.09 (dd, 2H), 2.83 (s, 3H), 2.45-2.37 (m, 1H), 2.32-2.25 (m, 1H), 2.25-2.12 (m, 2H), 1.89 (s, 2H), 1.85-1.78 (m, 1H), 1.78-1.69 (m, 1H), 1.60-1.47 (m, 2H).

### Example 55: Compound C20

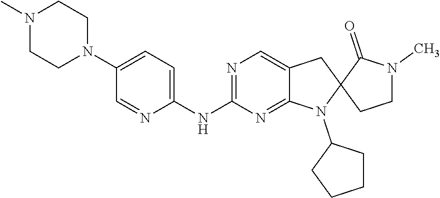

### Synthesis of 7′-cyclopentyl-1-methyl-2′-((5-(4-methylpiperazin-1-yl)pyridin-2-yl)amino)-5′,7′-dihydrospiro[pyrrolidine-3,6′-pyrrolo[2,3-d]pyrimidin]-2-one (Compound C20)

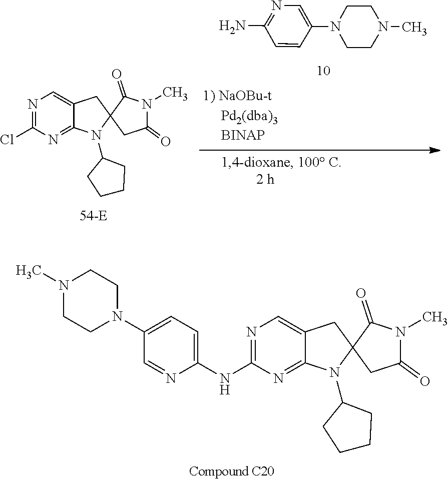

Compound C20 was synthesized following the general procedure B, 35 mg (29% yield). MS(ESI) m/z 463.3 (M+H)⁺. ¹H NMR (600 MHz, DMSO-d₆) δ 8.55 (s, 1H), 8.01 (s, 1H), 7.92 (d, J=2.9 Hz, 1H), 7.62 (s, 1H), 7.36 (dd, J=9.1, 2.5 Hz, 1H), 3.46-3.33 (m, 4H), 3.09 (s, 4H), 2.93 (q, J=16.6 Hz, 2H), 2.80 (s, 3H), 2.48 (s, 4H), 2.35-2.24 (m, 2H), 2.23 (s, 3H), 2.19-2.12 (m, 1H), 1.89-1.80 (m, 2H), 1.74-1.62 (m, 2H), 1.57-1.41 (m, 2H).

### Example 56: Compound C3

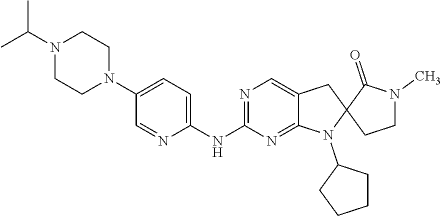 [SMILES: CC(C)N1CCN(c2ccc(Nc3ncc4c(n3)N(C3CCCC3)C3(CCN(C)C3=O)C4)nc2)CC1]

### Synthesis of 7′-cyclopentyl-2′-((5-(4-isopropylpiperazin-1-yl)pyridin-2-yl)amino)-1-methyl-5′,7′-dihydrospiro[pyrrolidine-3,6′-pyrrolo[2,3-d]pyrimidin]-2-one (Compound C3)

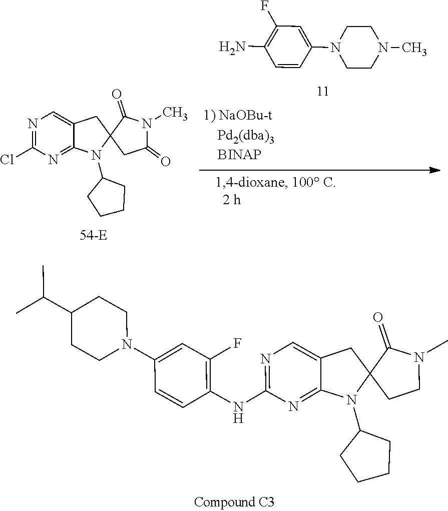 [SMILES: CN1CCN(c2ccc(N)c(F)c2)CC1 ; CN1C(=O)CC2(Cc3cnc(Cl)nc3N2C2CCCC2)C1=O ; CC(C)C1CCN(c2ccc(Nc3ncc4c(n3)N(C3CCCC3)C3(CCN(C)C3=O)C4)c(F)c2)CC1]

Compound C3 was synthesized following the general procedure B, 33 mg (27% yield). MS(ESI) m/z 491.3 (M+H)⁺.

### Example 57: Compound C2

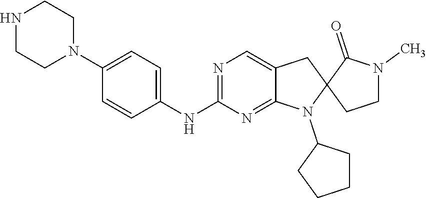 [SMILES: CN1CCC2(Cc3cnc(Nc4ccc(N5CCNCC5)cc4)nc3N2C2CCCC2)C1=O]

### Synthesis of 7′-cyclopentyl-1-methyl-2′-((4-(piperazin-1-yl)phenyl)amino)-5′,7′-dihydrospiro[pyrrolidine-3,6′-pyrrolo[2,3-d]pyrimidin]-2-one (Compound C2)

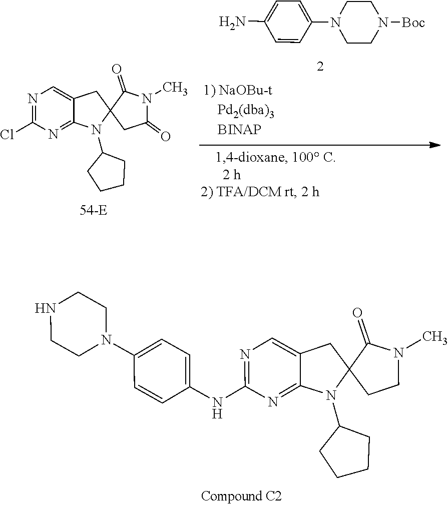 [SMILES: CC(C)(C)OC(=O)N1CCN(c2ccc(N)cc2)CC1 ; CN1C(=O)CC2(Cc3cnc(Cl)nc3N2C2CCCC2)C1=O ; CN1CCC2(Cc3cnc(Nc4ccc(N5CCNCC5)cc4)nc3N2C2CCCC2)C1=O]

Compound C2 was synthesized following the general procedure A, 32 mg (44% yield). MS(ESI) m/z 448.3 (M+H)⁺.

### Example 58: Compound C1

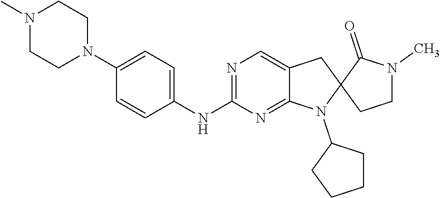

### Synthesis of 7′-cyclopentyl-1-methyl-2′-((4-(4-methylpiperazin-1-yl)phenyl)amino)-5′,7′-dihydrospiro[pyrrolidine-3,6′-pyrrolo[2,3-d]pyrimidin]-2-one (Compound C1)

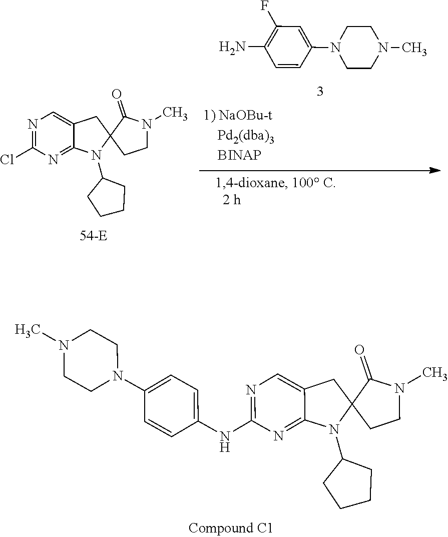

Compound C1 was synthesized following the general procedure B, 4 mg (5% yield). MS(ESI) m/z 462.3 (M+H)⁺.

### Example 59: Compound C4

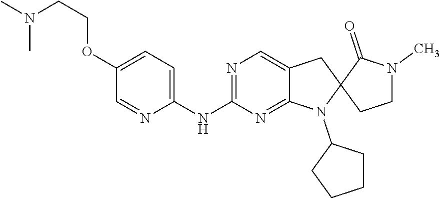

### Synthesis of 7′-cyclopentyl-2′-((5-(2-(dimethylamino)ethoxy)pyridin-2-yl)amino)-1-methyl-5′,7′-dihydrospiro[pyrrolidine-3,6′-pyrrolo[2,3-d]pyrimidin]-2-one (Compound C4)

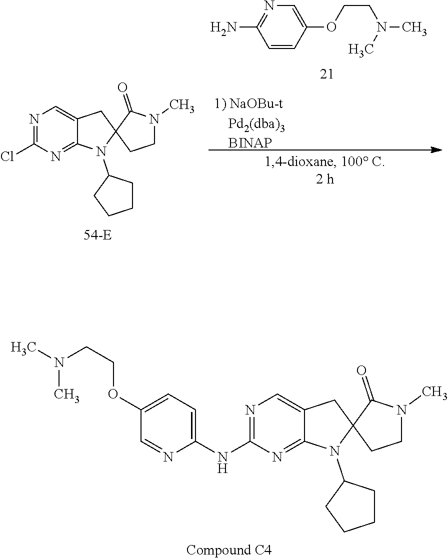

Compound C4 was synthesized following the general procedure B, 14 mg (12% yield). MS(ESI) m/z 452.2 (M+H)⁺.

### Example 60: Compound C5

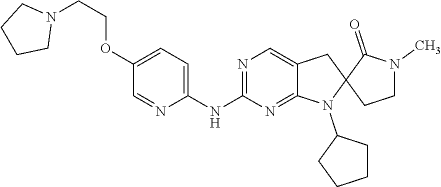 [SMILES: CN1CCC2(Cc3cnc(Nc4ccc(OCCN5CCCC5)cn4)nc3N2C2CCCC2)C1=O]

### Synthesis of 7′-cyclopentyl-1-methyl-2′-((5-(2-(pyrrolidin-1-yl)ethoxy)pyridin-2-yl)amino)-5′,7′-dihydrospiro[pyrrolidine-3,6′-pyrrolo[2,3-d]pyrimidin]-2-one (Compound C5)

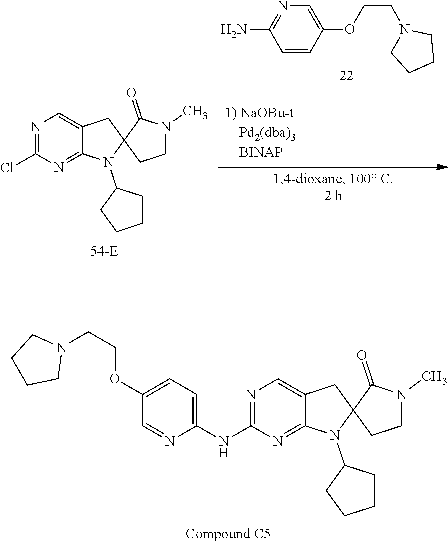 [SMILES: Nc1ccc(OCCN2CCCC2)cn1 ; CN1CCC2(Cc3cnc(Cl)nc3N2C2CCCC2)C1=O ; CN1CCC2(Cc3cnc(Nc4ccc(OCCN5CCCC5)cn4)nc3N2C2CCCC2)C1=O]

Compound C5 was synthesized following the general procedure B, 43 mg (36% yield). MS(ESI) m/z 478.2 (M+H)⁺.

### Example 61: Compound C6

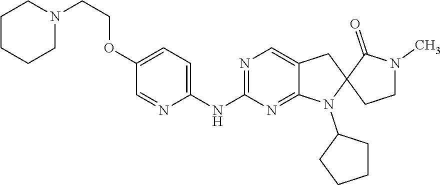 [SMILES: CN1CCC2(Cc3cnc(Nc4ccc(OCCN5CCCCC5)cn4)nc3N2C2CCCC2)C1=O]

### Synthesis of 7′-cyclopentyl 1-methyl-2′-((5-(2-(piperidin-1-yl)ethoxy)pyridin-2-yl)amino)-5′,7′-dihydrospiro[pyrrolidine-3,6′-pyrrolo[2,3-d]pyrimidin]-2-one (Compound C6)

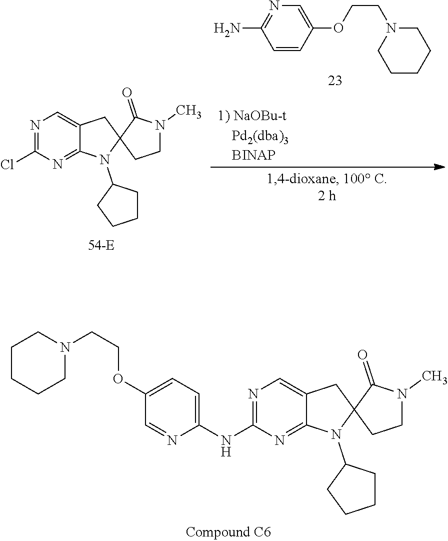 [SMILES: Nc1ccc(OCCN2CCCCC2)cn1 ; CN1CCC2(Cc3cnc(Cl)nc3N2C2CCCC2)C1=O ; CN1CCC2(Cc3cnc(Nc4ccc(OCCN5CCCCC5)cn4)nc3N2C2CCCC2)C1=O]

Compound C6 was synthesized following the general procedure B, 19 mg (15% yield). MS(ESI) m/z 492.2 (M+H)⁺.

### Example 62: Compound C15

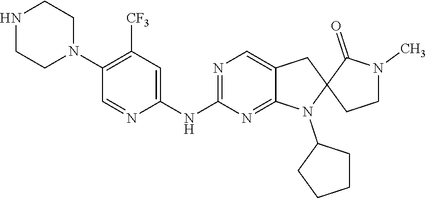 [SMILES: CN1CCC2(Cc3cnc(Nc4cc(C(F)(F)F)c(N5CCNCC5)cn4)nc3N2C2CCCC2)C1=O]

### Synthesis of 7′-cyclopentyl-1-methyl-2′-((5-(piperazin-1-yl)-4-(trifluoromethyl)pyridin-2-yl)amino)-5′,7′-dihydrospiro[pyrrolidine-3,6′-pyrrolo[2,3-d]pyrimidin]-2-one (Compound C15)

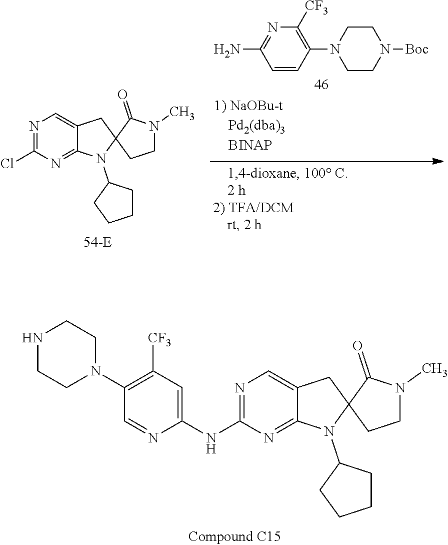 [SMILES: CC(C)(C)OC(=O)N1CCN(c2ccc(N)nc2C(F)(F)F)CC1 ; CN1CCC2(Cc3cnc(Cl)nc3N2C2CCCC2)C1=O ; CN1CCC2(Cc3cnc(Nc4cc(C(F)(F)F)c(N5CCNCC5)cn4)nc3N2C2CCCC2)C1=O]

Compound C15 was synthesized following the general procedure A, 51 mg (40% yield). MS(ESI) m/z 517.3 (M+H)⁺.

### Example 63: Compound C13

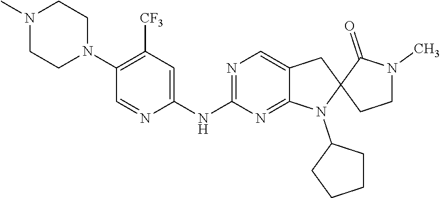

### Synthesis of 7′-cyclopentyl-1-methyl-2′-((5-(4-methylpiperazin-1-yl)-4-(trifluoromethyl)pyridin-2-yl)amino)-5′,7′-dihydrospiro[pyrrolidine-3,6′-pyrrolo[2,3-d]pyrimidin]-2-one (Compound C13)

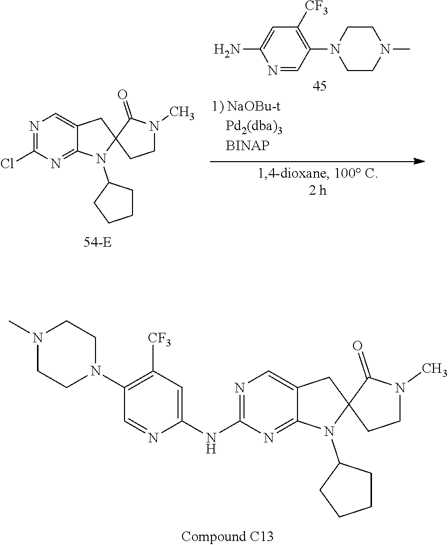

Compound C13 was synthesized following the general procedure B, 17 mg (20% yield). MS(ESI) m/z 531.2 (M+H)⁺.

### Example 64: Compound C8

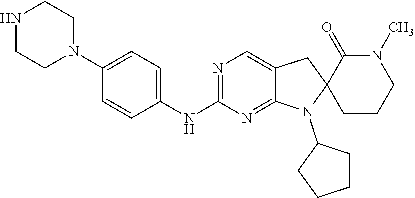 [SMILES: CN1CCCC2(Cc3cnc(Nc4ccc(N5CCNCC5)cc4)nc3N2C2CCCC2)C1=O]

### Synthesis of N-methyl-3-(phenylselanyl)propan-1-amine (64-F)

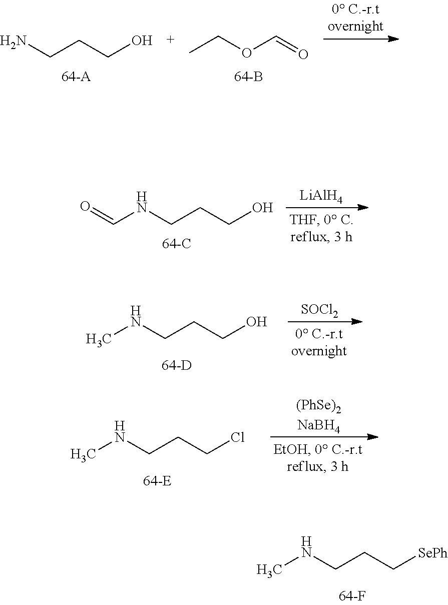

Synthesis of 64-C: (Ref: WO 2004004648): 3-amino-1-propanol 64-A (100 g mL, 1331 mmol) and ethyl formate 64-B (296 g, 3994 mmol) were combined and stirred at room temperature for 24 hours. The excess ethyl formate was removed under reduced pressure to give the title compound 64-C (142 g, crude) which was used such for the next step. MS(ESI) m/z 104.2 [M+H]⁺. ¹H NMR (600 MHz, Chloroform-d) δ 8.16 (s, 1H), 3.67 (t, J=5.7 Hz, 2H), 3.43 (q, J=6.2 Hz, 2H), 1.74 (m, 2H).

Synthesis of 64-D: (Ref: WO 2004004648): 64-C (25 g, 242 mmol) in 250 mL of THF was added to a suspension LAH (10 g, 267 mmol) in THF at 0° C. The mixture was heated at reflux for 3 hours. Water was added (11 mL) followed by 10% aq. NaOH solution (33 mL) and water (11 mL). The resulting solid was removed via filtration through Celite and the solid was washed with additional THF. The filtrate was collected and concentrated under reduced pressure to give the title compound 64-D, which was used for the next step without further purification. (17 g, 78% yield).

Synthesis of 64-E: Prepared according to the procedure described for the preparation of Compound 54-B. (21 g, 86%). MS(ESI) m/z 108.1 [M+H]⁺.

Synthesis of 64-F: Prepared according to the procedure described for the preparation of Compound 54-B. (13.7 g, 41% yield). MS(ESI) m/z 230.1 (M+H)⁺. ¹H NMR (600 MHz, Chloroform-d) δ 7.54-7.44 (m, 2H), 7.31-7.19 (m, 3H), 2.96 (t, J=7.2 Hz, 2H), 2.73 (s, 1H), 2.46 (bs, 5H), 1.99-1.88 (m, 2H).

### Step 2: Synthesis of 2′-chloro-7′-cyclopentyl-1-methyl-5′,7′-dihydrospiro[piperidine-3,6′-pyrrolo[2,3-d]pyrimidin]-2-one (64-H)

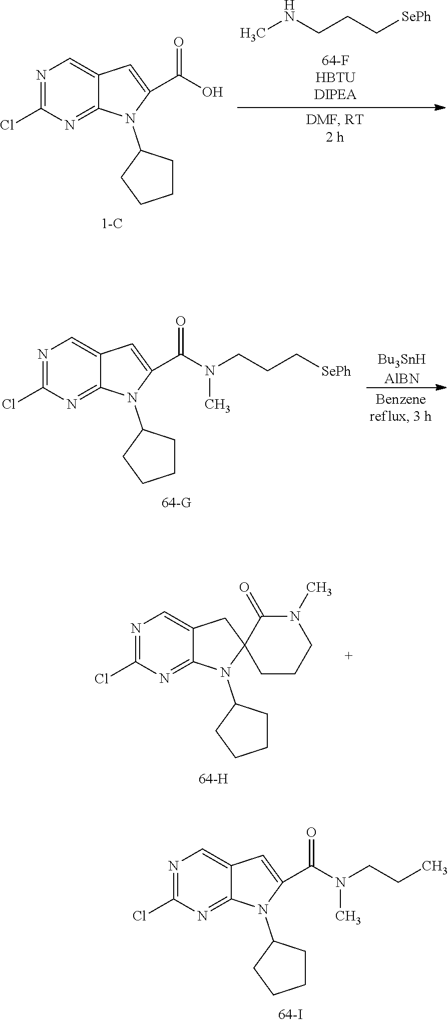

Synthesis of 64-G: Prepared according to the procedure described for the preparation of Compound 1-D. (5.6 g, 79% yield). MS(ESI) m/z 477.1 [M+H]⁺. ¹H NMR (600 MHz, Chloroform-d) δ 8.78 (d, J=12.0 Hz, 1H), 7.54 (d, J=5.1 Hz, 1H), 7.33-7.21 (m, 4H), 6.50 (s, 0.5H), 6.41 (s, 0.5H), 4.90-4.74 (m, 1H), 3.69-3.67 (m, 1H), 3.50-3.40 (m, 1H), 3.06 (d, J=43.3 Hz, 3H), 2.97 (t, J=6.6 Hz, 1H), 2.74 (t, J=6.3 Hz, 1H), 2.33 (s, 2H), 2.16-1.89 (m, 6H), 1.70-1.62 (m, 2H).

### Synthesis of 64-H and 64-I: Prepared according to the procedure described for the preparation of Compound 1-F and 1-G

Compound 64-H: (0.9 g, 24% yield). MS(ESI) m/z 321.11 [M+H]⁺. ¹H NMR (600 MHz, Chloroform-d) δ 7.61 (s, 1H), 3.53-3.44 (m, 2H), 3.33-3.27 (m, 1H), 3.21 (d, J=16.3 Hz, 1H), 2.99 (s, 3H), 2.92-2.87 (m, 1H), 2.32-2.24 (m, 1H), 2.20-2.15 (m, 1H), 2.10-2.05 (m, 1H), 2.00-1.95 (m, 3H), 1.94-1.89 (m, 2H), 1.79-1.69 (m, 2H), 1.58-1.45 (m, 2H).

Compound 64-I: (1.6 g, 42% yield). MS(ESI) m/z 321.1 [M+H]⁺. ¹H NMR (600 MHz, Chloroform-d) δ 8.80 (s, 1H), 6.51 (d, J=12.8 Hz, 1H), 4.93-4.88 (m, 0.5H), 4.83-4.77 (m, 0.5H), 3.55 (t, J=7.4 Hz, 1H), 3.33 (t, J=7.3 Hz, 1H), 3.13 (s, 1.5H), 3.05 (s, 1.5H), 2.46-2.30 (m, 2H), 2.04 (s, 4H), 1.82-1.53 (m, 4H), 1.01 (t, J=7.3 Hz, 1H), 0.83 (t, J=7.3 Hz, 2H).

### Step 3: Synthesis of 7′-cyclopentyl-1-methyl-2′-((4-(piperazin-1-yl)phenyl)amino)-5′,7′-dihydrospiro[piperidine-3,6′-pyrrolo[2,3-d]pyrimidin]-2-one (Compound C8)

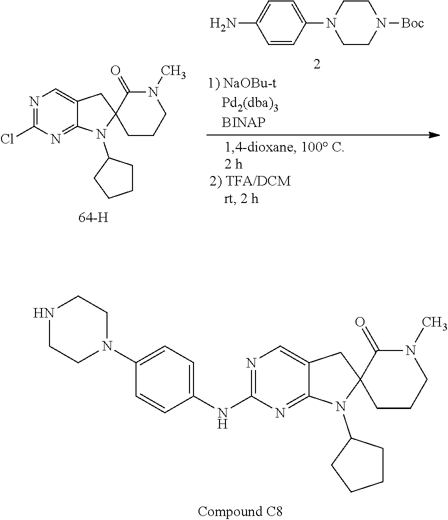 [SMILES: CC(C)(C)OC(=O)N1CCN(c2ccc(N)cc2)CC1 ; CN1CCCC2(Cc3cnc(Cl)nc3N2C2CCCC2)C1=O ; CN1CCCC2(Cc3cnc(Nc4ccc(N5CCNCC5)cc4)nc3N2C2CCCC2)C1=O]

Compound C8 was synthesized following the general procedure A, 14 mg (12% yield). MS(ESI) m/z 462.2 (M+H)⁺.

### Example 65: Compound C7

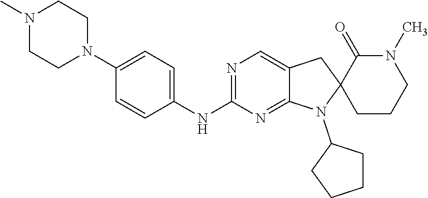

### Synthesis of 7′-cyclopentyl-1-methyl-2′-((4-(4-methylpiperazin-1-yl)phenyl)amino)-5′,7′-dihydrospiro[piperidine-3,6′-pyrrolo[2,3-d]pyrimidin]-2-one (Compound C7)

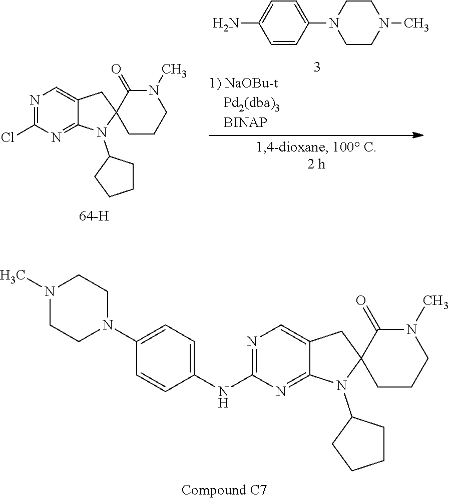

Compound C7 was synthesized following the general procedure B, 14 mg (12% yield). MS(ESI) m/z 476.2 (M+H)⁺.

### Example 66: Compound C25

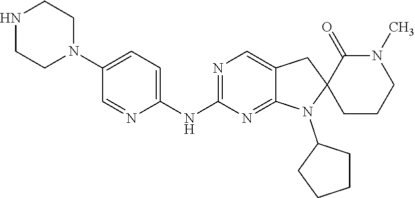 [SMILES: CN1CCCC2(Cc3cnc(Nc4ccc(N5CCNCC5)cn4)nc3N2C2CCCC2)C1=O]

### Synthesis of 7′-cyclopentyl-1-methyl-2′-((5-(piperazin-1-yl)pyridin-2-yl)amino)-5′,7′-dihydrospiro[piperidine-3,6′-pyrrolo[2,3-d]pyrimidin]-2-one (Compound C25)

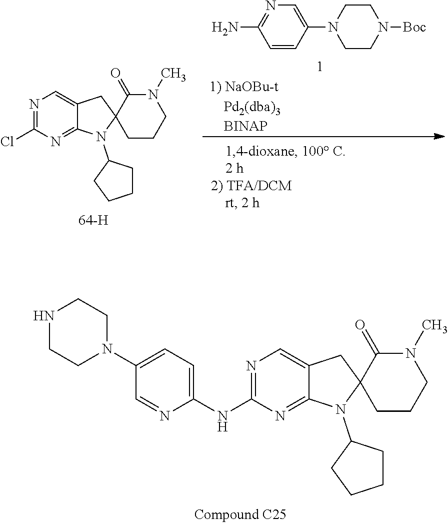 [SMILES: CC(C)(C)OC(=O)N1CCN(c2ccc(N)nc2)CC1 ; CN1CCCC2(Cc3cnc(Cl)nc3N2C2CCCC2)C1=O ; CN1CCCC2(Cc3cnc(Nc4ccc(N5CCNCC5)cn4)nc3N2C2CCCC2)C1=O]

Compound C25 was synthesized following the general procedure A, 72 mg (60% yield). MS(ESI) m/z 463.3 (M+H)⁺. ¹H NMR (600 MHz, DMSO-d₆) δ 8.90 (bs, 2H), 8.00 (d, J=2.8 Hz, 1H), 7.69 (dd, J=9.1, 2.8 Hz, 1H), 7.66 (s, 1H), 7.31 (d, J=7.6 Hz, 1H), 3.72 (p, J=8.8 Hz, 1H), 3.53-3.45 (m, 1H), 3.40-3.36 (m, 4H), 3.30-3.24 (m, 5H), 3.08 (s, 2H), 2.87 (s, 3H), 2.35-2.27 (m, 1H), 2.27-2.19 (m, 1H), 2.08-1.94 (m, 2H), 1.94-1.83 (m, 4H), 1.81-1.72 (m, 2H), 1.58-1.47 (m, 2H).

### Example 67: Compound C26

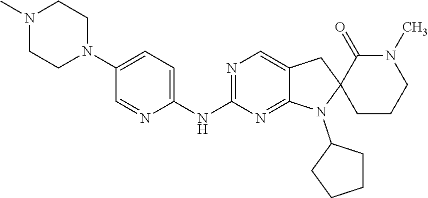

### Synthesis of 7′-cyclopentyl-1-methyl-2′-((5-(4-methylpiperazin-1-yl)pyridin-2-yl)amino)-5′,7′-dihydrospiro[piperidine-3,6′-pyrrolo[2,3-d]pyrimidin]-2-one (Compound C26)

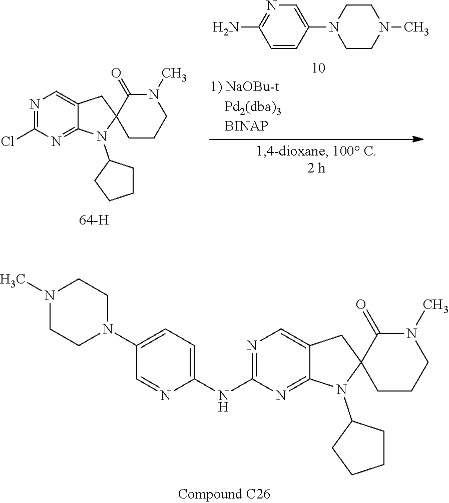

Compound C26 was synthesized following the general procedure B, 20 mg (16% yield). MS(ESI) m/z 477.3 (M+H)⁺. ¹H NMR (600 MHz, DMSO-d₆) δ 8.43 (s, 1H), 8.05 (d, J=9.0 Hz, 1H), 7.91 (d, J=2.8 Hz, 1H), 7.55 (s, 1H), 7.34 (dd, J=9.1, 2.9 Hz, 1H), 3.52-3.40 (m, 3H), 3.27-3.20 (m, 2H), 3.13-3.04 (m, 4H), 2.93 (s, 2H), 2.85 (s, 3H), 2.48-2.43 (m, 4H), 2.37-2.29 (m, 1H), 2.22 (s, 3H), 2.20-2.11 (m, 2H), 1.89-1.80 (m, 5H), 1.72-1.62 (m, 2H), 1.53-1.43 (m, 2H).

### Example 68: Compound C9

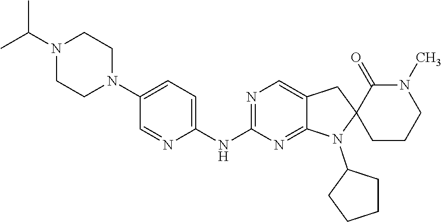 [SMILES: CC(C)N1CCN(c2ccc(Nc3ncc4c(n3)N(C3CCCC3)C3(CCCN(C)C3=O)C4)nc2)CC1]

### Synthesis of 7′-cyclopentyl-2′-((5-(4-isopropylpiperazin-1-yl)pyridin-2-yl)amino)-1-methyl-5′,7′-dihydrospiro[piperidine-3,6′-pyrrolo[2,3-d]pyrimidin]-2-one (Compound C9)

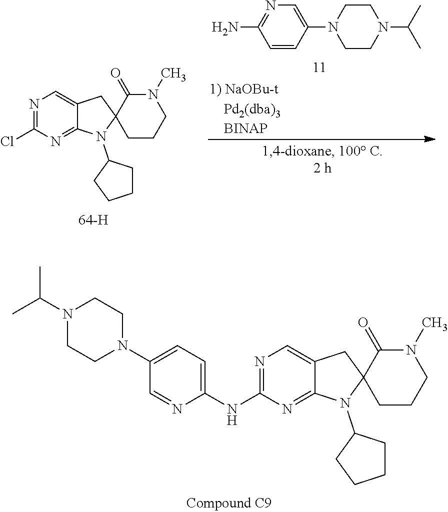 [SMILES: CC(C)N1CCN(c2ccc(N)nc2)CC1 ; CN1CCCC2(Cc3cnc(Cl)nc3N2C2CCCC2)C1=O ; CC(C)N1CCN(c2ccc(Nc3ncc4c(n3)N(C3CCCC3)C3(CCCN(C)C3=O)C4)nc2)CC1]

Compound C9 was synthesized following the general procedure B, 8 mg (6% yield). MS(ESI) m/z 505.3 (M+H)⁺.

### Example 69: Compound C10

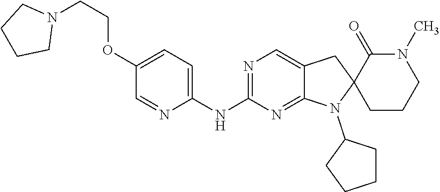 [SMILES: CN1CCCC2(Cc3cnc(Nc4ccc(OCCN5CCCC5)cn4)nc3N2C2CCCC2)C1=O]

### Synthesis of 7′-cyclopentyl-1-methyl-2′-((5-(2-(pyrrolidin-1-yl)ethoxy)pyridin-2-yl)amino)-5′,7′-dihydrospiro[piperidine-3,6′-pyrrolo[2,3-d]pyrimidin]-2-one (Compound C10)

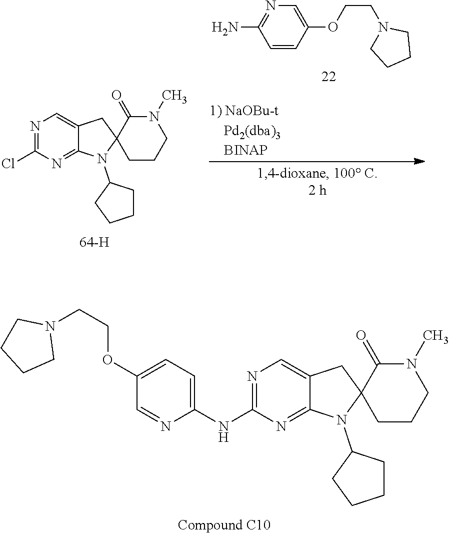 [SMILES: Nc1ccc(OCCN2CCCC2)cn1 ; CN1CCCC2(Cc3cnc(Cl)nc3N2C2CCCC2)C1=O ; CN1CCCC2(Cc3cnc(Nc4ccc(OCCN5CCCC5)cn4)nc3N2C2CCCC2)C1=O]

Compound C10 was synthesized following the general procedure B, 17 mg (14% yield). MS(ESI) m/z 492.3 (M+H)⁺.

### Example 70: Compound C11

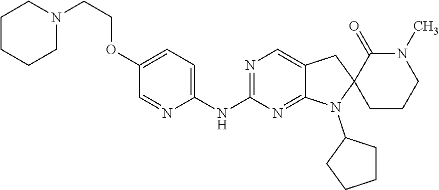 [SMILES: CN1CCCC2(Cc3cnc(Nc4ccc(OCCN5CCCCC5)cn4)nc3N2C2CCCC2)C1=O]

### Synthesis of 7′-cyclopentyl-1-methyl-2′-((5-(2-(piperidin-1-yl)ethoxy)pyridin-2-yl)amino)-5′,7′-dihydrospiro[piperidine-3,6′-pyrrolo[2,3-d]pyrimidin]-2-one (Compound C11)

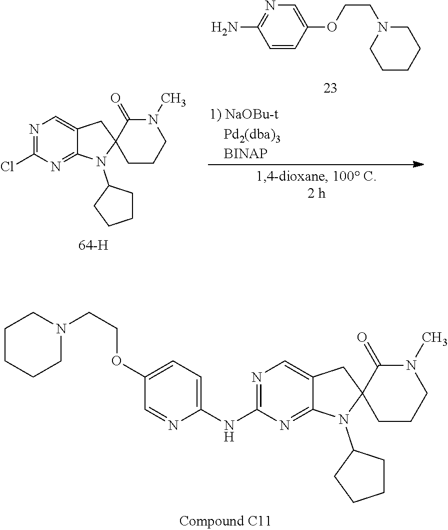 [SMILES: Nc1ccc(OCCN2CCCCC2)cn1 ; CN1CCCC2(Cc3cnc(Cl)nc3N2C2CCCC2)C1=O ; CN1CCCC2(Cc3cnc(Nc4ccc(OCCN5CCCCC5)cn4)nc3N2C2CCCC2)C1=O]

Compound C11 was synthesized following the general procedure B, 4 mg (3% yield). MS(ESI) m/z 506.3 (M+H)⁺.

### Example 71: Compound C65

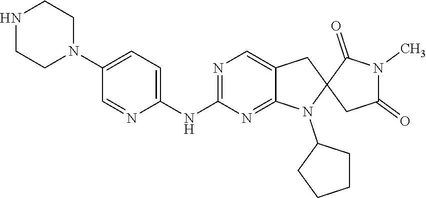 [SMILES: CN1C(=O)CC2(Cc3cnc(Nc4ccc(N5CCNCC5)cn4)nc3N2C2CCCC2)C1=O]

### Step 1: Synthesis of N-methyl-2-(phenylselanyl)acetamide (71-B)

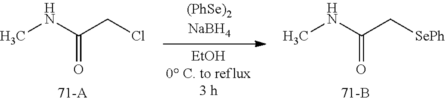

Synthesis of 71-B: (Ref: Kyei et al., Tetrahedron Lett. 45(48): 8931-8934 (2004)): NaBH₄ (8 g, 210 mmol) was added portion wise to a stirred solution of diphenyldiselenide (22 g, 70 mmol) in absolute EtOH (200 mL) under argon at 0° C. After complete addition of NaBH₄, within 15 min the reaction medium became colorless. When the evolution of hydrogen ceased, a solution of 71-A (15 g, 140 mmol) in absolute EtOH (200 mL) was added dropwise at 0° C. The reaction mixture was then stirred at reflux for 3 h. Upon completion of the reaction, brine (20 mL) was added and the solvent was evaporated under vacuum. The residue was dissolved in EtOAc (500 mL) and washed with saturated aqueous sodium hydrogen carbonate, water, and then brine. The organic layer was dried over Na₂SO₄, evaporated under vacuum and then purified by silica gel column chromatography (hexane:ethyl acetate, 3:2) to give 71-B (29 g, 91% yield). MS(ESI) m/z 230.1 (M+H)⁺. ¹H NMR (600 MHz, Chloroform-d) δ 7.52-7.41 (m, 2H), 7.30-7.27 (m, 3H), 6.50 (s, 1H), 3.55 (s, 2H), 2.79 (d, J=4.9 Hz, 3H).

### Step 2: Synthesis of 2′-chloro-7′-cyclopentyl-1-methyl-5′,7′-dihydrospiro[pyrrolidine-3,6′-pyrrolo[2,3-d]pyrimidine]-2,5-dione (71-D)

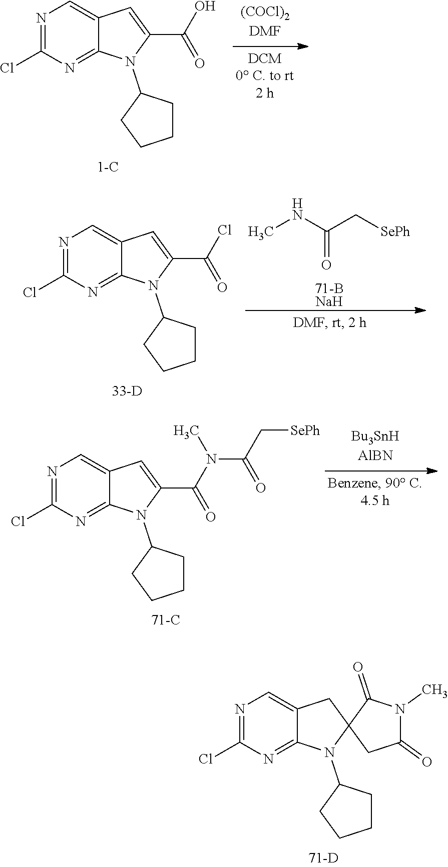

Synthesis of 71-C: (Ref: US 20110218342): 71-B was dissolved in 150 mL of dry THF. This was followed by the addition of NaH (1.5 g, 62.5 mmol) at 0° C. After stirring the reaction for 30 minutes at RT, acid chloride 33-D (dissolved in 150 mL of DCM) was added at 0° C., and the reaction was stirred at RT for overnight. After completion of the reaction, it was quenched by the addition of 30 mL of 1 N HCl. The reaction mixture was extracted with ethyl aceate. The organic layer was washed with saturated sodium bicarbonate, dried over Na₂SO₄, and purified by flash chromatography using silica gel (20% ethyl acetate:hexane) to yield desired product 71-C (4.1 g, 34% yield). MS(ESI) m/z 477.1 (M+H)⁺.

Synthesis of 71-D: (Ref: Kyei et al., Tetrahedron Lett. 45(48): 8931-8934 (2004)): A solution of tributyltin hydride (3.6 mL, 13.2 mmol) and 2,2′-azobis(isobutyronitrile) (300 mg, 20 mole percent) in benzene (150 mL) was added drop wise to a solution of 71-C (4.2 g, 8.8 mmol) in benzene (150 mL) at 90° C. over 1.5 hours. The reaction solution was stirred at the same temperature for three hours. The reaction solution was cooled to room temperature and poured onto water, followed by extraction with ethyl acetate. The combined organic extracts were concentrated on vaccuo and purified on column (1% MeOH in DCM) to give 71-D (1.2 g, 43%). MS(ESI) m/z 321.1 (M+H)⁺. ¹H NMR (600 MHz, Chloroform-d) δ 7.77 (s, 1H), 3.48 (dd, J=16.6, 1.4 Hz, 1H), 3.43-3.34 (m, 1H), 3.11 (s, 3H), 3.09-3.00 (m, 2H), 2.89 (d, J=18.6 Hz, 1H), 2.21-218 (m, 2H), 2.00-1.89 (m, 2H), 1.80-186 (m, 2H), 1.56-1.47 (m, 2H).

### Step 3: Synthesis of 7′-cyclopentyl-1-methyl-2′-((5-(piperazin-1-yl)pyridin-2-yl)amino)-5′,7′-dihydrospiro[pyrrolidine-3,6′-pyrrolo[2,3-d]pyrimidine]-2,5-dione (Compound C65)

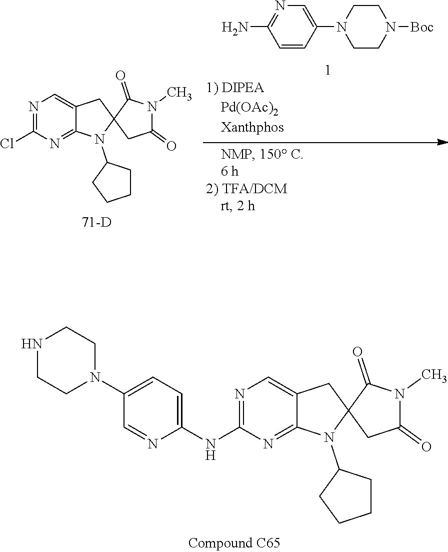 [SMILES: CC(C)(C)OC(=O)N1CCN(c2ccc(N)nc2)CC1 ; CN1C(=O)CC2(Cc3cnc(Cl)nc3N2C2CCCC2)C1=O ; CN1C(=O)CC2(Cc3cnc(Nc4ccc(N5CCNCC5)cn4)nc3N2C2CCCC2)C1=O]

Compound C65 was synthesized following the general procedure I, 2 mg (1% yield). MS(ESI) m/z 463.3 (M+H)⁺.

### Example 72: Compound C66

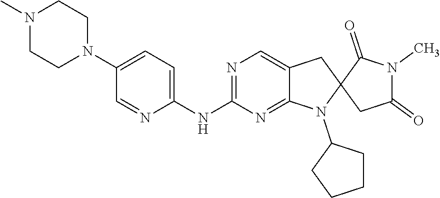

### Synthesis of 7′-cyclopentyl-1-methyl-2′-((5-(4-methylpiperazin-1-yl)pyridin-2-yl)amino)-5′,7′-dihydrospiro[pyrrolidine-3,6′-pyrrolo[2,3-d]pyrimidine]-2,5-dione (Compound C66)

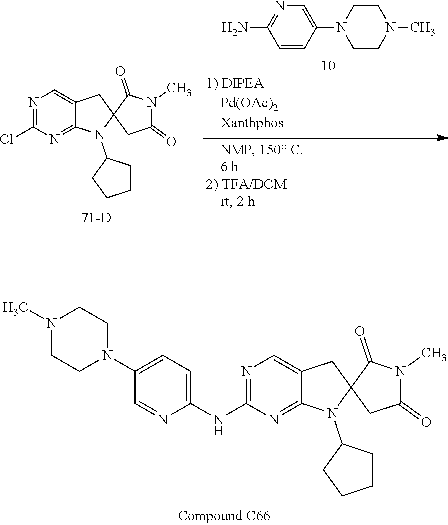

Compound C66 was synthesized following the general procedure I, 16 mg (11% yield). MS(ESI) m/z 477.3 (M+H)⁺.

### Example 73 (SPV-221)

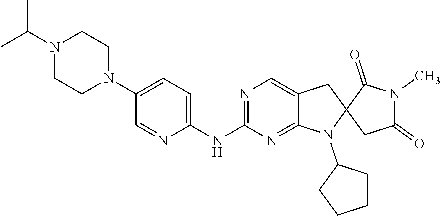 [SMILES: CC(C)N1CCN(c2ccc(Nc3ncc4c(n3)N(C3CCCC3)C3(CC(=O)N(C)C3=O)C4)nc2)CC1]

### Synthesis of 7′-cyclopentyl-2′-((5-(4-isopropylpiperazin-1-yl)pyridin-2-yl)amino)-1-methyl-5′,7′-dihydrospiro[pyrrolidine-3,6′-pyrrolo[2,3-d]pyrimidine]-2,5-dione (Compound C67)

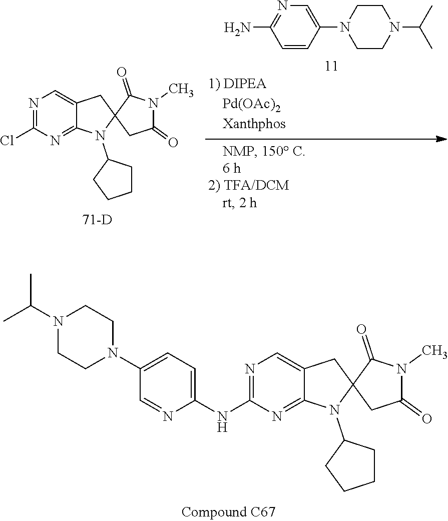 [SMILES: CC(C)N1CCN(c2ccc(N)nc2)CC1 ; CN1C(=O)CC2(Cc3cnc(Cl)nc3N2C2CCCC2)C1=O ; CC(C)N1CCN(c2ccc(Nc3ncc4c(n3)N(C3CCCC3)C3(CC(=O)N(C)C3=O)C4)nc2)CC1]

Compound C67 was synthesized following the general procedure I, 5 mg (3% yield). MS(ESI) m/z 505.3 (M+H)⁺. ¹H NMR (600 MHz, Chloroform-d) δ 8.24 (d, J=9.1 Hz, 1H), 8.13 (s, 1H), 7.99 (d, J=2.7 Hz, 1H), 7.62 (s, 1H), 7.34 (s, 1H), 5.02 (d, J=6.8 Hz, 1H), 4.47-4.42 (m, 1H), 3.48 (d, J=1.9 Hz, 2H), 3.20-3.16 (m, 4H), 3.12 (s, 3H), 2.76-2.72 (m, 1H), 2.72-2.68 (m, 4H), 2.19-2.09 (m, 3H), 1.78 (s, 3H), 1.68 (d, J=3.9 Hz, 3H), 1.10 (d, J=6.5 Hz, 6H).

### Example 74: Compound C68

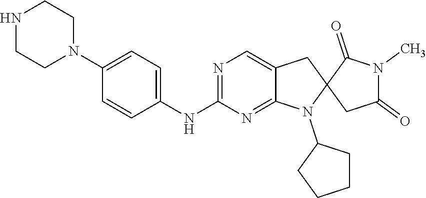 [SMILES: CN1C(=O)CC2(Cc3cnc(Nc4ccc(N5CCNCC5)cc4)nc3N2C2CCCC2)C1=O]

### Synthesis of 7′-cyclopentyl-1-methyl-2′-((4-(piperazin-1-yl)phenyl)amino)-5′,7′-dihydrospiro[pyrrolidine-3,6′-pyrrolo[2,3-d]pyrimidine]-2,5-dione (Compound C68)

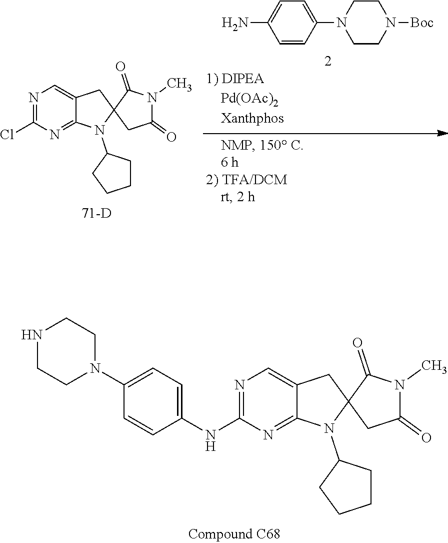 [SMILES: CC(C)(C)OC(=O)N1CCN(c2ccc(N)cc2)CC1 ; CN1C(=O)CC2(Cc3cnc(Cl)nc3N2C2CCCC2)C1=O ; CN1C(=O)CC2(Cc3cnc(Nc4ccc(N5CCNCC5)cc4)nc3N2C2CCCC2)C1=O]

Compound C68 was synthesized following the general procedure I, 17 mg (11% yield). MS(ESI) m/z 462.3 (M+H)⁺. ¹H NMR (600 MHz, Chloroform-d) δ 8.07 (s, 1H), 7.51 (d, J=8.8 Hz, 2H), 7.33 (s, 1H), 6.92 (d, J=9.0 Hz, 2H), 5.00 (d, J=6.3 Hz, 1H), 4.51-4.39 (m, 1H), 3.46 (s, 2H), 3.16-3.09 (m, 6H), 3.10-3.02 (m, 4H), 2.19-2.10 (m, 2H), 1.83-1.73 (m, 3H), 1.71-1.63 (m, 2H), 1.54-1.43 (m, 3H).

### Example 75: Compound C69

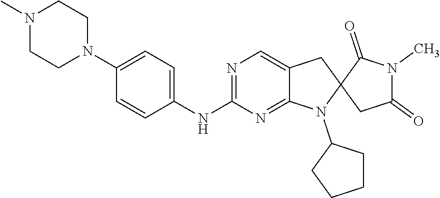

### Synthesis of 7′-cyclopentyl-1-methyl-2′-((4-(4-methylpiperazin-1-yl)phenyl)amino)-5′,7′-dihydrospiro[pyrrolidine-3,6′-pyrrolo[2,3-d]pyrimidine]-2,5-dione (Compound C69)

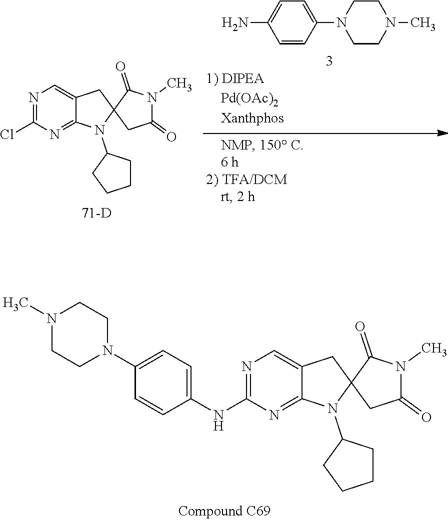

Compound C69 was synthesized following the general procedure I, 3 mg (2% yield). MS(ESI) m/z 476.3 (M+H)⁺. ¹H NMR (600 MHz, Chloroform-d) δ 8.08 (s, 1H), 7.50 (d, J=8.8 Hz, 2H), 7.33 (s, 1H), 6.92 (d, J=8.9 Hz, 2H), 5.00 (s, 1H), 4.49-4.40 (m, 1H), 3.47 (s, 2H), 3.23 (s, 4H), 3.11 (s, 3H), 2.67 (s, 4H), 2.42 (s, 3H), 2.21-2.08 (m, 3H), 1.77 (s, 3H), 1.71-1.64 (m, 3H).

### Example 76: Compound C70

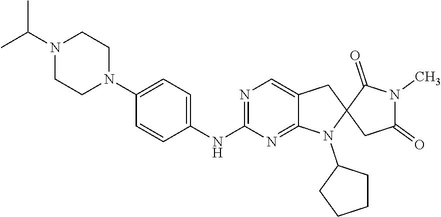 [SMILES: CC(C)N1CCN(c2ccc(Nc3ncc4c(n3)N(C3CCCC3)C3(CC(=O)N(C)C3=O)C4)cc2)CC1]

### Synthesis of 7′-cyclopentyl-2′-((4-(4-isopropylpiperazin-1-yl)phenyl)amino)-1-methyl-5′,7′-dihydrospiro[pyrrolidine-3,6′-pyrrolo[2,3-d]pyrimidine]-2,5-dione (Compound C70)

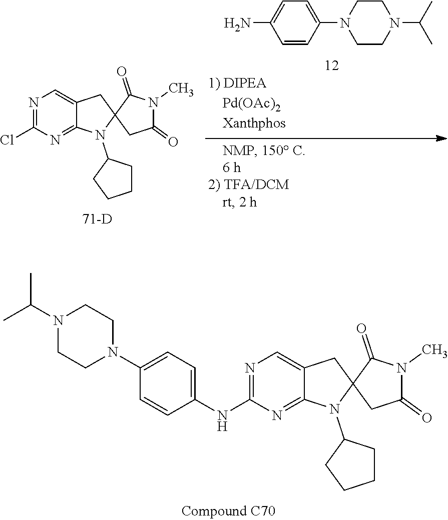 [SMILES: CC(C)N1CCN(c2ccc(N)cc2)CC1 ; CN1C(=O)CC2(Cc3cnc(Cl)nc3N2C2CCCC2)C1=O ; CC(C)N1CCN(c2ccc(Nc3ncc4c(n3)N(C3CCCC3)C3(CC(=O)N(C)C3=O)C4)cc2)CC1]

Compound C70 was synthesized following the general procedure 1, 10 mg (8% yield). MS(ESI) m/z 504.3 (M+H)⁺. ¹H NMR (600 MHz, Chloroform-d) δ 8.08 (s, 1H), 7.49 (d, J=8.6 Hz, 2H), 7.33 (s, 1H), 6.92 (d, J=8.9 Hz, 2H), 4.99 (d, J=6.5 Hz, 1H), 4.52-4.36 (m, 1H), 3.50-3.43 (m, 2H), 3.19 (s, 4H), 3.11 (s, 3H), 2.71 (s, 5H), 2.20-2.03 (m, 2H), 1.80-1.71 (m, 2H), 1.71-1.62 (m, 2H), 1.53-1.45 (m, 3H), 1.11 (s, 6H).

### Example 77: Compound C71

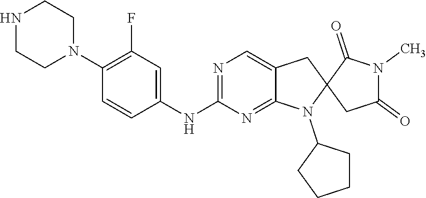 [SMILES: CN1C(=O)CC2(Cc3cnc(Nc4ccc(N5CCNCC5)c(F)c4)nc3N2C2CCCC2)C1=O]

### Synthesis of 7′-cyclopentyl-2′-((3-fluoro-4-(piperazin-1-yl)phenyl)amino)-1-methyl-5′,7′-dihydrospiro[pyrrolidine-3,6′-pyrrolo[2,3-d]pyrimidine]-2,5-dione (Compound C71)

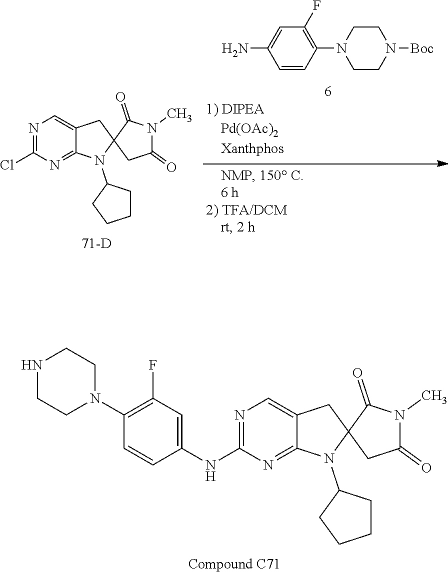 [SMILES: CC(C)(C)OC(=O)N1CCN(c2ccc(N)cc2F)CC1 ; CN1C(=O)CC2(Cc3cnc(Cl)nc3N2C2CCCC2)C1=O ; CN1C(=O)CC2(Cc3cnc(Nc4ccc(N5CCNCC5)c(F)c4)nc3N2C2CCCC2)C1=O]

Compound C71 was synthesized following the general procedure I, 20 mg (17% yield). MS(ESI) m/z 480.3 (M+H)⁺. ¹H NMR (600 MHz, Chloroform-d) δ 8.07 (s, 1H), 7.76 (d, J=17.0 Hz, 1H), 7.33 (s, 1H), 7.06 (d, J=11.2 Hz, 1H), 6.91 (t, J=8.9 Hz, 2H), 5.04 (s, 1H), 4.48-4.43 (m, 1H), 3.47 (s, 2H), 3.12 (s, 3H), 3.09-3.00 (m, 8H), 2.21-2.12 (m, 3H), 1.80-1.75 (m, 3H), 1.72-1.66 (m, 4H).

### Example 78: Compound C72

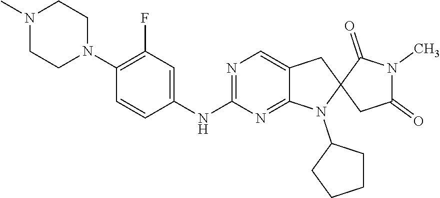

### Synthesis of 7′-cyclopentyl-2′-((3-fluoro-4-(4-methylpiperazin-1-yl)phenyl)amino)-1-methyl-5′,7′-dihydrospiro[pyrrolidine-3,6′-pyrrolo[2,3-d]pyrimidine]-2,5-dione (Compound C72)

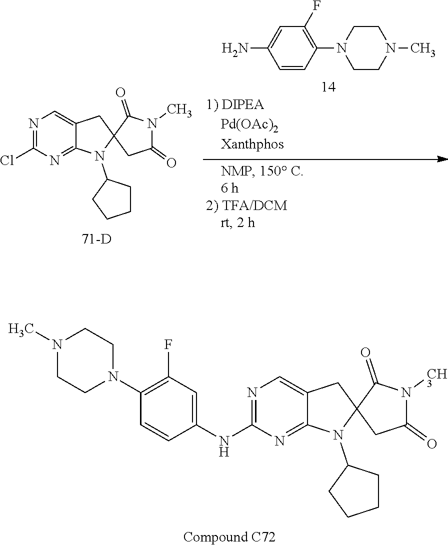

Compound C72 was synthesized following the general procedure I, 5 mg (4% yield). MS(ESI) m/z 494.3 (M+H)⁺. ¹H NMR (600 MHz, Chloroform-d) δ 8.08 (s, 1H), 7.75 (d, J=14.4 Hz, 1H), 7.33 (s, 1H), 7.07-7.03 (m, 1H), 6.95-6.88 (m, 1H), 5.07-5.01 (m, 1H), 4.50-4.39 (m, 1H), 3.47 (s, 2H), 3.12 (s, 6H), 2.65 (s, 4H), 2.39 (s, 3H), 2.21-2.11 (m, 2H), 1.82-1.73 (m, 2H), 1.74-1.65 (m, 3H), 1.55-1.47 (m, 3H).

### Example 79: Compound C73

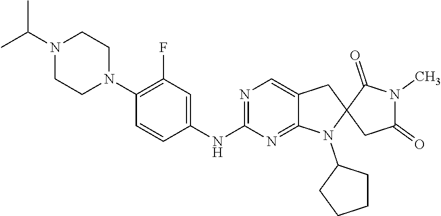 [SMILES: CC(C)N1CCN(c2ccc(Nc3ncc4c(n3)N(C3CCCC3)C3(CC(=O)N(C)C3=O)C4)cc2F)CC1]

### Synthesis of 7′-cyclopentyl-2′-((3-fluoro-4-(4-isopropylpiperazin-1-yl)phenyl)amino)-1-methyl-5′,7′-dihydrospiro[pyrrolidine-3,6′-pyrrolo[2,3-d]pyrimidine]-2,5-dione (Compound C73)

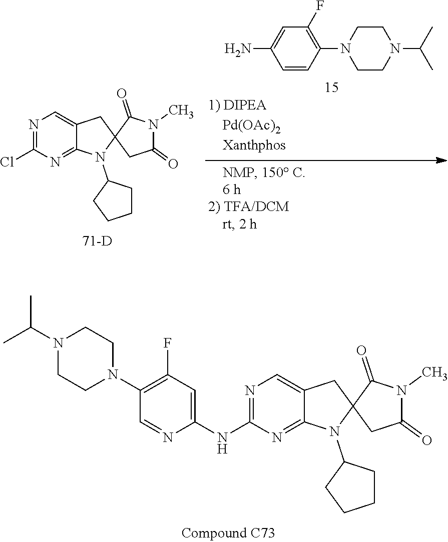 [SMILES: CC(C)N1CCN(c2ccc(N)cc2F)CC1 ; CN1C(=O)CC2(Cc3cnc(Cl)nc3N2C2CCCC2)C1=O ; CC(C)N1CCN(c2cnc(Nc3ncc4c(n3)N(C3CCCC3)C3(CC(=O)N(C)C3=O)C4)cc2F)CC1]

Compound C73 was synthesized following the general procedure I, 4 mg (3% yield). MS(ESI) m/z 522.3 (M+H)⁺. ¹H NMR (600 MHz, Chloroform-d) δ 8.08 (s, 1H), 7.75 (d, J=14.3 Hz, 1H), 7.33 (s, 1H), 7.04 (d, J=6.4 Hz, 1H), 6.91 (t, J=9.1 Hz, 1H), 5.03 (d, J=6.5 Hz, 1H), 4.48-4.40 (m, 1H), 3.47 (s, 2H), 3.15-3.06 (m, 6H), 2.72 (s, 5H), 2.22-2.11 (m, 2H), 1.84-1.74 (m, 2H), 1.73-1.65 (m, 2H), 1.55-1.46 (m, 4H), 1.10 (d, J=6.0 Hz, 6H).

### Example 80: Compound C21

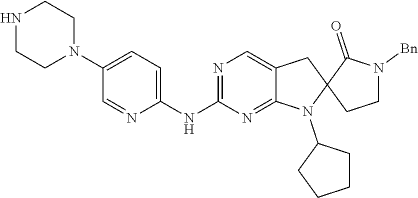 [SMILES: O=C1N(Cc2ccccc2)CCC12Cc1cnc(Nc3ccc(N4CCNCC4)cn3)nc1N2C1CCCC1]

Step 1: Synthesis of 1-benzyl-2′-chloro-7′-cyclopentyl-5′,7′-dihydrospiro[pyrrolidine-3,6′-pyrrolo[2,3-d]pyrimidin]-2-one (80-E)

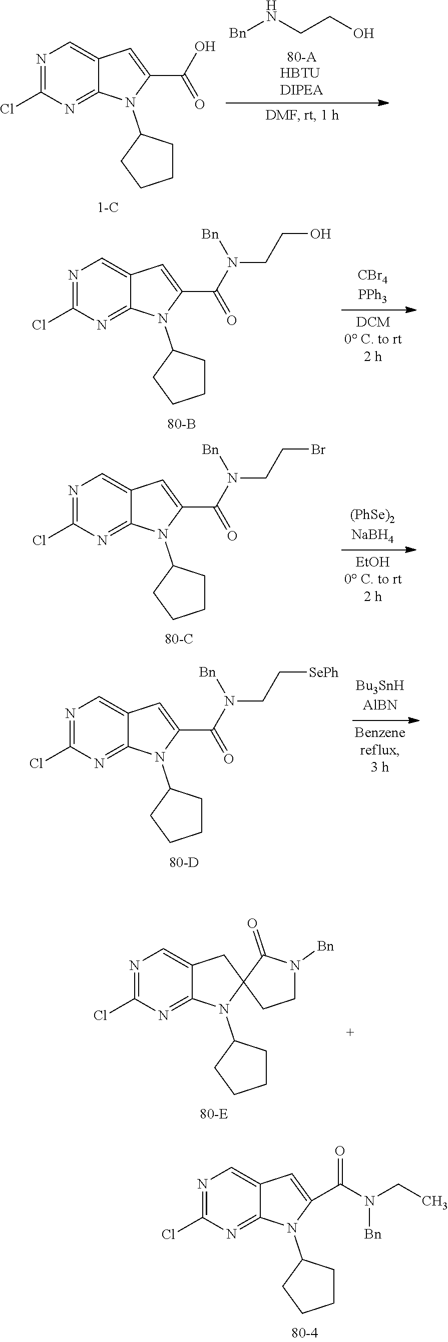

Synthesis of 80-B: Prepared according to the procedure described for the preparation of Compound 1-D. (2.8 g, 94% yield). MS(ESI) m/z 399.1 (M+H)⁺.

Synthesis of 80-C: (Ref: WO 2016112088): 80-B (2.8 g, 7.0 mmol) and Tetrabromomethane (2.8 g, 8.4 mmol) were dissolved in DCM under inert atmosphere. Triphenylphosphine (2.8 g, 10.5 mmol) was added portion wise to this at 0° C. over 30 min and then stirred at room temperature for 2 h. After completion, the reaction was diluted with DCM and washed with saturated aqueous sodium hydrogen carbonate, water, then brine. The organic layer was dried over Na₂SO₄, and the solvent was evaporated under reduced pressure and then purified using column chromatography (ethyl acetate:hexane) to compound 80-D (1.3 g, 40% yield). MS(ESI) m/z 461.1 (M+H)⁺.

Synthesis of 80-D: Prepared according to the procedure described for the preparation of Compound 71-B. (1.1 g 73% yield). MS(ESI) m/z 539.1 (M+H)⁺. ¹H NMR (600 MHz, Chloroform-d) δ 8.74 (s, 0.5H), 8.67 (s, 0.5H), 7.48 (s, 1H), 7.38-7.30 (m, 3H), 7.30-7.27 (m, 2H), 7.19 (d, J=6.9 Hz, 1H), 7.14-7.00 (m, 2H), 6.94 (d, J=6.6 Hz, 1H), 6.49 (s, 0.5H), 6.36 (s, 0.5H), 4.99-4.88 (m, 1H), 4.79-4.60 (m, 3H), 3.77 (s, 1H), 3.48 (s, 1H), 3.21 (s, 1H), 2.82 (d, J=7.0 Hz, 1H), 2.41 (s, 1H), 2.28 (s, 1H), 2.08 (s, 2H), 2.00 (s, 2H), 1.70 (s, 1H).

### Synthesis of 80-E and 80-F: Prepared according to the procedure described for the preparation of Compound 1-F and 1-G

Compound 80-E: 488 mg (61% yield). MS(ESI) m/z 383.1 (M+H)⁺. ¹H NMR (600 MHz, Chloroform-d) δ 7.67 (s, 1H), 7.40-7.30 (m, 3H), 7.26 (s, 1H), 7.25 (s, 1H), 4.51 (d, J=2.1 Hz, 2H), 3.50-3.39 (m, 1H), 3.32-3.19 (m, 3H), 2.91-2.84 (m, 1H), 2.37-2.26 (m, 2H), 2.23-2.15 (m, 1H), 2.16-2.09 (m, 1H), 1.97-1.83 (m, 3H), 1.75-1.67 (m, 1H), 1.55-1.45 (m, 2H).

Compound 80-F: 150 mg (19% yield). MS(ESI) m/z 383.2 (M+H)⁺. ¹H NMR (600 MHz, Chloroform-d) δ 8.80 (s, 0.5H), 8.72 (s, 0.5H), 7.42-7.30 (m, 4H), 7.18 (d, J=5.9 Hz, 1H), 6.56 (s, 0.5H), 6.50 (s, 0.5H), 5.02-4.82 (m, 1H), 4.80 (s, 1H), 4.63 (s, 1H), 3.69-3.53 (m, 1H), 3.40-3.24 (m, 1H), 2.36 (s, 2H), 2.17-1.94 (m, 5H), 1.74-1.63 (m, 2H), 1.16 (s, 2H).

### Step 2: Synthesis of 7′-cyclopentyl-2′-((3-fluoro-4-(4-isopropylpiperazin-1-yl)phenyl)amino)-1-methyl-5′,7′-dihydrospiro[pyrrolidine-3,6′-pyrrolo[2,3-d]pyrimidine]-2,5-dione (Compound C21)

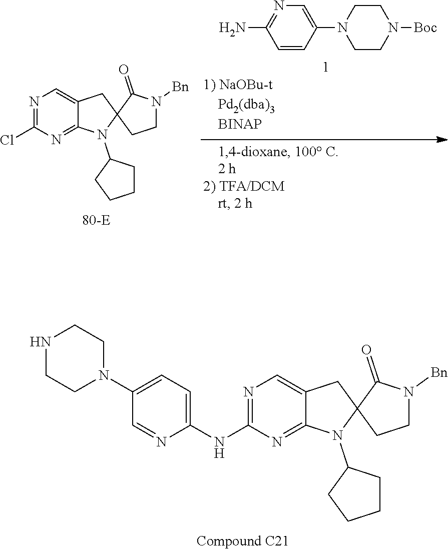 [SMILES: CC(C)(C)OC(=O)N1CCN(c2ccc(N)nc2)CC1 ; O=C1N(Cc2ccccc2)CCC12Cc1cnc(Cl)nc1N2C1CCCC1 ; O=C1N(Cc2ccccc2)CCC12Cc1cnc(Nc3ccc(N4CCNCC4)cn3)nc1N2C1CCCC1]

Compound C21 was synthesized following the general procedure A, 39 mg (29% yield). MS(ESI) m/z 525.3 (M+H)⁺. ¹H NMR (600 MHz, DMSO-d₆) δ 9.04 (bs, 2H), 8.01 (d, J=2.7 Hz, 1H), 7.74 (s, 1H), 7.69 (dd, J=9.1, 2.8 Hz, 1H), 7.43-7.36 (m, 2H), 7.36-7.30 (m, 2H), 7.27 (d, J=7.1 Hz, 2H), 4.56-4.40 (m, 2H), 3.66-3.53 (m, 1H), 3.43-3.37 (m, 5H), 3.31-3.25 (m, 5H), 3.20-3.09 (m, 2H), 2.48-2.41 (m, 1H), 2.34-2.27 (m, 1H), 2.27-2.11 (m, 2H), 1.94-1.85 (m, 2H), 1.85-1.78 (m, 1H), 1.78-1.70 (m, 1H), 1.59-1.40 (m, 2H).

### Example 81: Compound C22

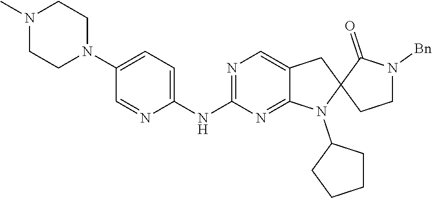 [SMILES: CN1CCN(c2ccc(Nc3ncc4c(n3)N(C3CCCC3)C3(CCN(Cc5ccccc5)C3=O)C4)nc2)CC1]

### Synthesis of 1-benzyl-7′-cyclopentyl-2′-((5-(4-methylpiperazin-1-yl)pyridin-2-yl)amino)-5′,7′-dihydrospiro[pyrrolidine-3,6′-pyrrolo[2,3-d]pyrimidin]-2-one (Compound C22)

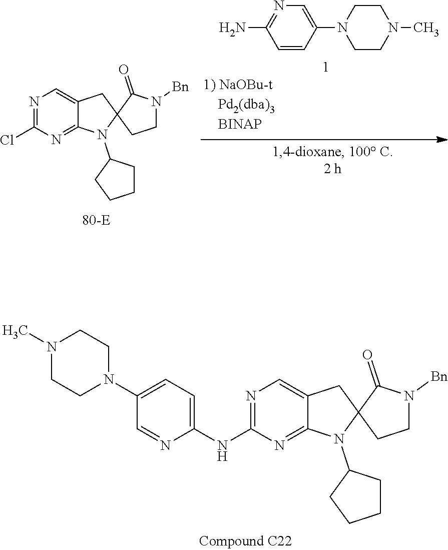 [SMILES: CN1CCN(c2ccc(N)nc2)CC1 ; O=C1N(Cc2ccccc2)CCC12Cc1cnc(Cl)nc1N2C1CCCC1 ; CN1CCN(c2ccc(Nc3ncc4c(n3)N(C3CCCC3)C3(CCN(Cc5ccccc5)C3=O)C4)nc2)CC1]

Compound C22 was synthesized following the general procedure B, 90 mg (64% yield). MS(ESI) m/z 539.3 (M+H)⁺. ¹H NMR (600 MHz, DMSO-d₆) δ 8.49 (s, 1H), 8.05 (d, J=9.1 Hz, 1H), 7.91 (d, J=2.9 Hz, 1H), 7.63 (s, 1H), 7.40-7.36 (m, 2H), 7.36-7.33 (m, 1H), 7.33-7.29 (m, 1H), 7.25 (d, J=7.2 Hz, 2H), 4.53-4.38 (m, 2H), 3.46-3.38 (m, 1H), 3.31-3.20 (m, 3H), 3.12-3.05 (m, 3H), 2.98 (s, 2H), 2.47-2.41 (m, 3H), 2.35-2.24 (m, 2H), 2.19-2.13 (m, 1H), 1.84 (s, 2H), 1.74-1.63 (m, 2H), 1.63-1.51 (m, 2H), 1.51-1.41 (m, 3H), 1.34-1.24 (m, 2H).

### Example 82: Compound C23

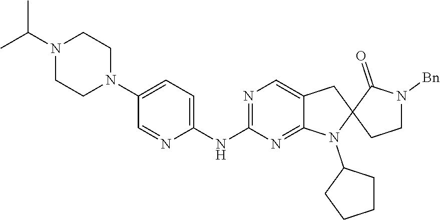 [SMILES: CC(C)N1CCN(c2ccc(Nc3ncc4c(n3)N(C3CCCC3)C3(CCN(Cc5ccccc5)C3=O)C4)nc2)CC1]

### Synthesis of 1-benzyl-7′-cyclopentyl-2′-((5-(4-isopropylpiperazin-1-yl)pyridin-2-yl)amino)-5′,7′-dihydrospiro[pyrrolidine-3,6′-pyrrolo[2,3-d]pyrimidin]-2-one (Compound C23)

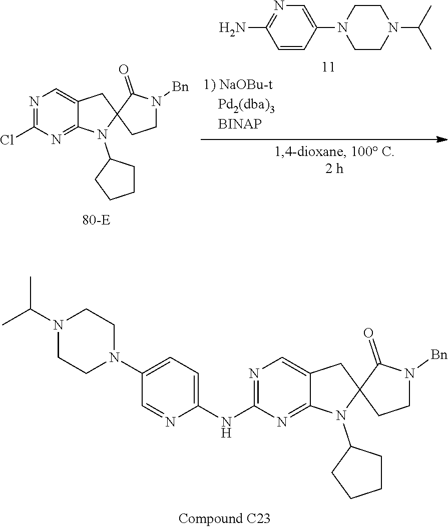 [SMILES: CC(C)N1CCN(c2ccc(N)nc2)CC1 ; O=C1N(Cc2ccccc2)CCC12Cc1cnc(Cl)nc1N2C1CCCC1 ; CC(C)N1CCN(c2ccc(Nc3ncc4c(n3)N(C3CCCC3)C3(CCN(Cc5ccccc5)C3=O)C4)nc2)CC1]

Compound C23 was synthesized following the general procedure B, 2 mg (2% yield). MS(ESI) m/z 567.3 (M+H)⁺. ¹H NMR (600 MHz, DMSO-d₆) δ 8.47 (s, 1H), 8.04 (d, J=9.1 Hz, 1H), 7.91 (d, J=2.9 Hz, 1H), 7.63 (s, 1H), 7.41-7.36 (m, 2H), 7.36-7.29 (m, 2H), 7.25 (d, J=7.1 Hz, 2H), 4.54-4.37 (m, 2H), 3.11-3.03 (m, 3H), 2.98 (s, 2H), 2.72-2.63 (m, 1H), 2.61 (s, 2H), 2.60-2.56 (m, 3H), 2.41-2.37 (m, 2H), 2.36-2.27 (m, 2H), 2.26-2.13 (m, 2H), 1.84 (s, 2H), 1.73-1.64 (m, 2H), 1.51-1.42 (m, 2H), 1.00 (d, J=6.5 Hz, 6H).

### Example 83: Compound C24

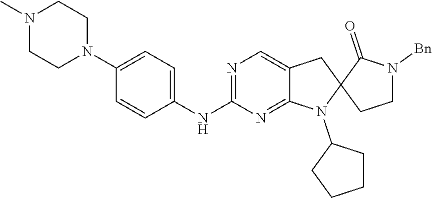 [SMILES: CN1CCN(c2ccc(Nc3ncc4c(n3)N(C3CCCC3)C3(CCN(Cc5ccccc5)C3=O)C4)cc2)CC1]

### Synthesis of 1-benzyl-7′-cyclopentyl-2′-((4-(4-methylpiperazin-1-yl)phenyl)amino)-5′,7′-dihydrospiro[pyrrolidine-3,6′-pyrrolo[2,3-d]pyrimidin]-2-one (Compound C24)

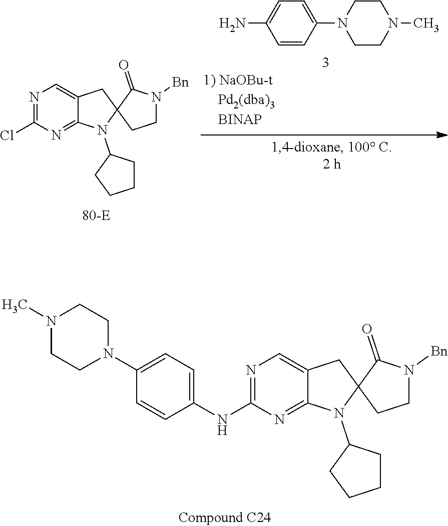 [SMILES: CN1CCN(c2ccc(N)cc2)CC1 ; O=C1N(Cc2ccccc2)CCC12Cc1cnc(Cl)nc1N2C1CCCC1 ; CN1CCN(c2ccc(Nc3ncc4c(n3)N(C3CCCC3)C3(CCN(Cc5ccccc5)C3=O)C4)cc2)CC1]

Compound C24 was synthesized following the general procedure B, 5 mg (4% yield). MS(ESI) m/z 538.3 (M+H)⁺.

### Example 84: Compound C39

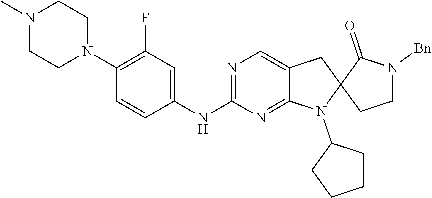 [SMILES: CN1CCN(c2ccc(Nc3ncc4c(n3)N(C3CCCC3)C3(CCN(Cc5ccccc5)C3=O)C4)cc2F)CC1]

Step 1: Synthesis of N-benzyl-2-(phenylselanyl)ethanamine (84-C)

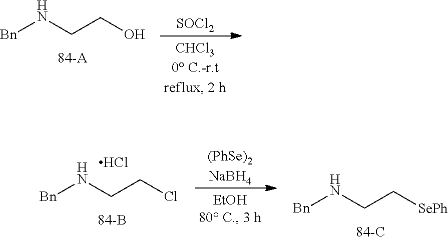

Synthesis of 84-B: (Ref: Habtemariam et al., J. Chem. Soc., Dalton Trans. 8: 1306-1318 (2001)): To a stirred solution of compound 84-A (20 g, 132 mmol) in chloroform (200 mL) at 0° C. was added thionyl chloride (12.5 mL, 172 mmol) in chloroform (100 mL) dropwise and stirred at RT for 30 min and at reflux for 2 h. Cooled the reaction mixture to RT and solid formed was filtered, washed with ether and dried to get compound 84-B (26 g, 96% yield) as a white solid. Lit. reported. MS(ESI) m/z 170.1 [M+H]⁺. ¹H NMR (600 MHz, DMSO-d₆) δ 9.76 (s, 2H), 7.68-7.54 (m, 2H), 7.53-7.36 (m, 3H), 4.19 (s, 2H), 3.96 (t, J=6.5 Hz, 2H), 3.26 (t, J=6.4 Hz, 2H).

Synthesis of 84-C: Prepared according to the procedure described for the preparation of compound 1-B. (30 g, 85% yield) as a white solid. MS(ESI) m/z 292.1 [M+H]⁺. ¹H NMR (600 MHz, Chloroform-d) δ 7.50-7.46 (m, 2H), 7.33-7.26 (m, 4H), 7.26-7.21 (m, 4H), 3.78 (s, 2H), 3.06 (t, J=6.6 Hz, 2H), 2.89 (t, J=6.6 Hz, 2H).

### Step 2: Synthesis of 1-benzyl-7′-cyclopentyl-2′-(methylthio)-5′,7′-dihydrospiro [pyrrolidine-3,6′-pyrrolo[2,3-d]pyrimidin]-2-one (84-E)

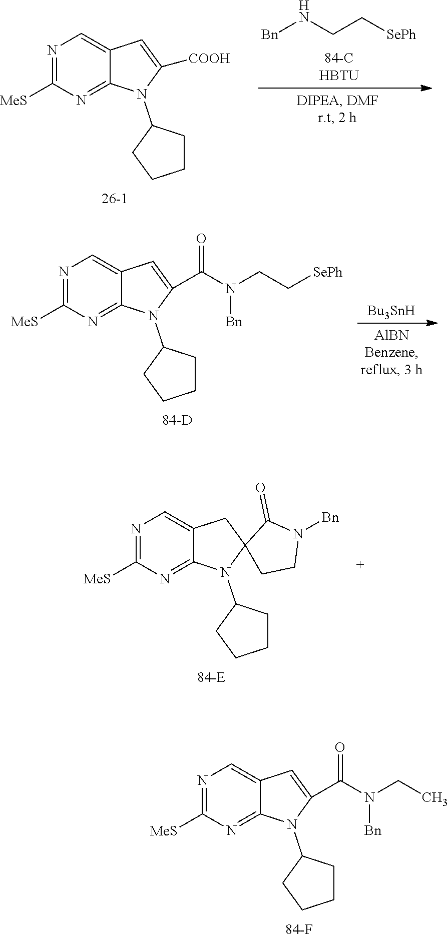

Synthesis of 84-D: Prepared according to the procedure described for the preparation of compound 1-D. (3.5 g, 88% yield). MS(ESI) m/z 292.1 [M+H]⁺. ¹H NMR (600 MHz, Chloroform-d) δ 8.66 (d, J=25.3 Hz, 1H), 7.48 (s, 1H), 7.41-7.22 (m, 5H), 7.21-6.84 (m, 4H), 6.37 (d, J=76.4 Hz, 1H), 4.98-4.57 (m, 3H), 3.76 (s, 1H), 3.54 (s, 1H), 3.21 (s, 1H), 2.82 (s, 1H), 2.61 (s, 3H), 2.57-2.30 (m, 2H), 2.19-1.92 (m, 2H), 1.67 (s, 1H).

Synthesis of 84-E and 84-F: Prepared according to the procedure described for the preparation of compounds 1-F and 1-G.

Compound 84-E: (0.85 g, 41% yield). MS(ESI) m/z 395.2 [M+H]⁺. ¹H NMR (600 MHz, Chloroform-d) δ 7.68 (s, 1H), 7.38-7.32 (m, 4H), 7.25 (s, 1H), 4.55-4.48 (m, 2H), 3.44-3.39 (m, 1H), 3.28-3.20 (m, 4H), 2.87-2.82 (m, 1H), 2.48 (s, 3H), 2.32-2.25 (m, 2H), 2.11-2.09 (m, 1H), 1.90-1.85 (m, 3H), 1.73-1.64 (m, 1H), 1.54-1.50 (m, 2H).

Compound 84-F: MS(ESI) m/z 395.2 [M+H]⁺. ¹HNMR (600 MHz, CDCl₃) δ (ppm): ¹H NMR (600 MHz, Chloroform-d) δ 8.80-8.60 (m, 1H), 7.57-6.87 (m, 5H), 6.53-6.28 (m, 1H), 4.94-4.58 (m, 3H), 3.96-3.44 (m, 2H), 3.40-2.76 (m, 2H), 2.68-2.56 (m, 3H), 2.56-2.34 (m, 2H), 2.16-1.93 (m, 4H), 1.77-1.63 (m, 3H).

### Step 3: Synthesis of 1-benzyl-7′-cyclopentyl-2′-(methylsulfonyl)-5′,7′-dihydrospiro [pyrrolidine-3,6′-pyrrolo[2,3-d]pyrimidin]-2-one (84-G)

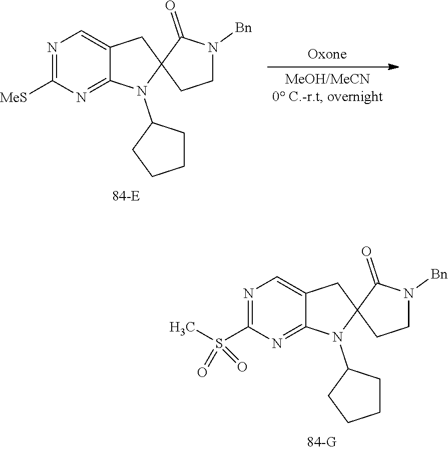 [SMILES: CSc1ncc2c(n1)N(C1CCCC1)C1(CCN(Cc3ccccc3)C1=O)C2 ; CS(=O)(=O)c1ncc2c(n1)N(C1CCCC1)C1(CCN(Cc3ccccc3)C1=O)C2]

Synthesis of 84-G: (Ref: WO 2006066748): To a stirred solution of compound 84-E (0.8 g, 2 mmol) in MeOH/MeCN (40 mL, 1:1) at 0° C. was added Oxone (3.1 g, 10 mmol) dissolved in 20 mL of H₂O dropwise and stirred at RT overnight. Removed solvents under reduced pressure and diluted with water and extracted with 20% MeOH in DCM (3×25 mL) and concentrated to get the crude compound which is purified by column chromatography (0 to 10% MeOH in DCM) to give compound 84-G (550 mg, 65% yield) as a light-yellow solid. MS(ESI) m/z 427.2 [M+H]⁺. ¹H NMR (600 MHz, Chloroform-d) δ 7.92 (s, 1H), 7.40-7.32 (m, 4H), 7.25 (s, 1H), 4.58-4.47 (m, 2H), 3.51-3.43 (m, 1H), 3.39-3.24 (m, 3H), 3.23 (s, 3H), 3.01-2.94 (m, 1H), 2.40-2.30 (m, 2H), 2.30-2.19 (m, 1H), 2.19-2.12 (m, 1H), 1.97-1.92 (m, 2H), 1.91-1.85 (m, 1H), 1.78-1.71 (m, 1H), 1.56-1.50 (m, 2H).

### Step 4: Synthesis of 1-benzyl-7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one (Compound C39)

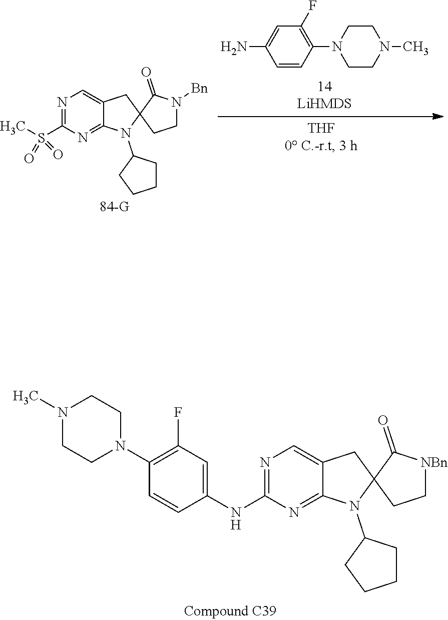 [SMILES: CN1CCN(c2ccc(N)cc2F)CC1 ; CS(=O)(=O)c1ncc2c(n1)N(C1CCCC1)C1(CCN(Cc3ccccc3)C1=O)C2 ; CN1CCN(c2ccc(Nc3ncc4c(n3)N(C3CCCC3)C3(CCN(Cc5ccccc5)C3=O)C4)cc2F)CC1]

Compound C39 was synthesized following the general procedure E, 5 mg (9% yield). MS(ESI) m/z 556.3 [M+H]⁺. ¹H NMR (600 MHz, Chloroform-d) δ 7.67 (dd, J=14.8, 2.2 Hz, 1H), 7.56 (s, 1H), 7.39-7.30 (m, 3H), 7.25 (s, 2H), 6.98 (d, J=7.1 Hz, 1H), 6.88 (t, J=9.1 Hz, 1H), 4.59-4.46 (m, 2H), 3.45-3.32 (m, 1H), 3.31-3.15 (m, 3H), 3.07 (s, 4H), 2.82 (d, J=15.5 Hz, 1H), 2.61 (s, 4H), 2.56-2.42 (m, 1H), 2.36 (s, 4H), 2.33-2.24 (m, 1H), 2.17-2.09 (m, 1H), 1.97-1.85 (m, 3H), 1.58-1.45 (m, 3H).

### Example 85: Compound C41

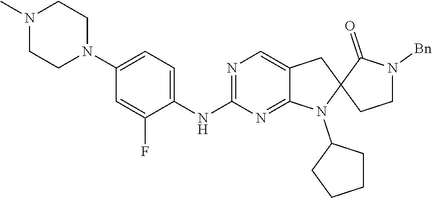 [SMILES: CN1CCN(c2ccc(Nc3ncc4c(n3)N(C3CCCC3)C3(CCN(Cc5ccccc5)C3=O)C4)c(F)c2)CC1]

### Synthesis of 1-benzyl-7′-cyclopentyl-2′-((2-fluoro-4-(4-methylpiperazin-1-yl)phenyl)amino)-5′,7′-dihydrospiro[pyrrolidine-3,6′-pyrrolo[2,3-d]pyrimidin]-2-one (Compound C41)

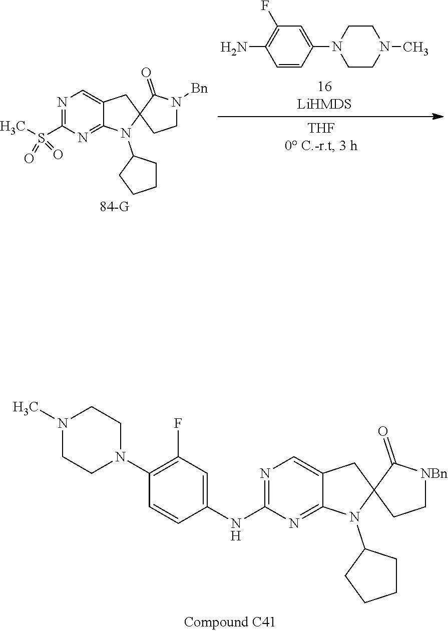 [SMILES: CN1CCN(c2ccc(N)c(F)c2)CC1 ; CS(=O)(=O)c1ncc2c(n1)N(C1CCCC1)C1(CCN(Cc3ccccc3)C1=O)C2 ; CN1CCN(c2ccc(Nc3ncc4c(n3)N(C3CCCC3)C3(CCN(Cc5ccccc5)C3=O)C4)cc2F)CC1]

Compound C41 was synthesized following the general procedure E, 16 mg (12% yield). MS(ESI) m/z 556.3 [M+H]⁺. ¹H NMR (600 MHz, Chloroform-d) δ 7.65 (d, J=14.7 Hz, 1H), 7.39-7.29 (m, 4H), 7.27 (s, 2H), 7.08 (d, J=9.5 Hz, 1H), 6.88 (t, J=9.1 Hz, 1H), 4.68-4.55 (m, 2H), 3.43-3.33 (m, 2H), 3.30-3.26 (m, 1H), 3.23 (d, J=15.8 Hz, 1H), 3.08 (s, 4H), 2.89 (d, J=15.7 Hz, 1H), 2.63 (s, 4H), 2.53-2.44 (m, 1H), 2.37 (s, 3H), 2.34-2.21 (m, 2H), 2.20-2.09 (m, 2H), 1.96-1.91 (m, 2H), 1.75-1.63 (m, 1H), 1.56-1.47 (m, 2H).

### Example 86: Compound C27

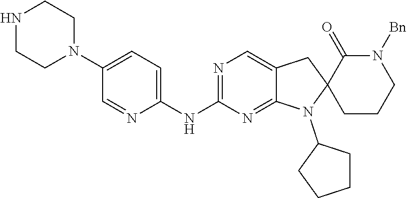 [SMILES: O=C1N(Cc2ccccc2)CCCC12Cc1cnc(Nc3ccc(N4CCNCC4)cn3)nc1N2C1CCCC1]

### Step 1: Synthesis of 1-benzyl-2′-chloro-7′-cyclopentyl-5′,7′-dihydrospiro[piperidine-3,6′-pyrrolo[2,3-d]pyrimidin]-2-one (86-E)

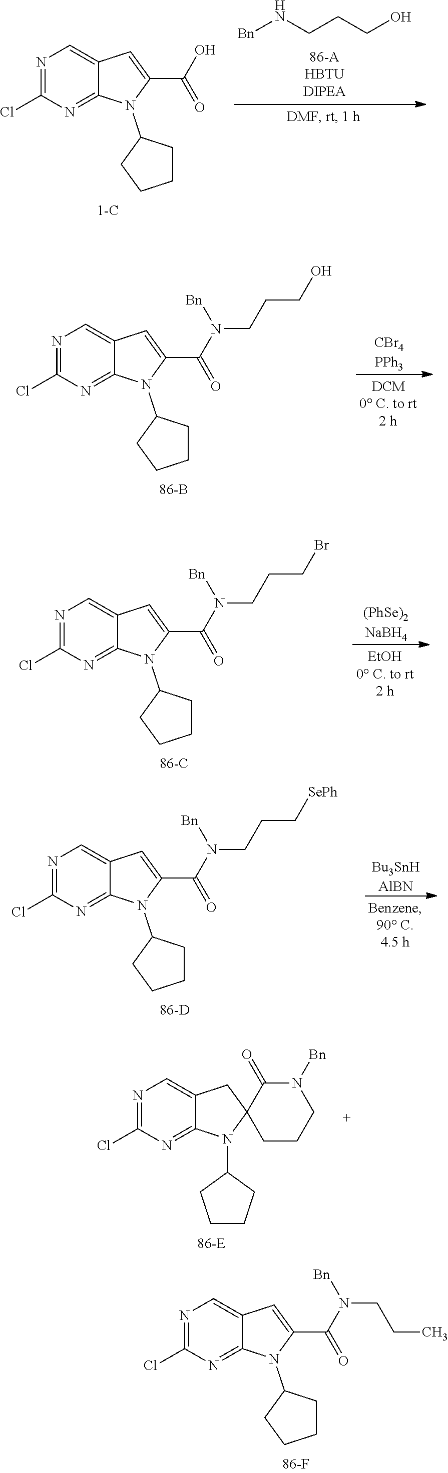

Synthesis of 86-B: Prepared according to the procedure described for the preparation of compound 1-D. (2.6 g, 87% yield). MS(ESI) m/z 413.2 (M+H)⁺. ¹H NMR (600 MHz, Chloroform-d) δ 8.74 (s, 1H), 7.44-7.30 (m, 3H), 7.18 (d, J=7.3 Hz, 2H), 6.55 (s, 1H), 5.05-4.84 (m, 1H), 4.68 (s, 2H), 3.77-3.62 (m, 3H), 2.81 (s, 1H), 2.48-2.30 (m, 2H), 2.15-1.98 (m, 4H), 1.90-1.79 (m, 2H), 1.74-1.66 (m, 3H).

Synthesis of 86-C: Prepared according to the procedure described for the preparation of compound 80-C. (1.0 g, 33% yield).

Synthesis of 86-D: Prepared according to the procedure described for the preparation of Compound 71-B. (1.0 g 87% yield). MS(ESI) m/z 533.1 (M+H)⁺. ¹H NMR (600 MHz, Chloroform-d) δ 8.74 (d, J=17.9 Hz, 1H), 7.48 (s, 1H), 7.41-7.30 (m, 3H), 7.26 (s, 4H), 7.22-7.17 (m, 1H), 7.17-7.09 (m, 1H), 6.47 (d, J=26.9 Hz, 1H), 4.92-4.82 (m, 1H), 4.73 (s, 2H), 4.60 (s, 1H), 3.63 (s, 1H), 3.34 (s, 1H), 2.92 (s, 1H), 2.67 (s, 1H), 2.42-2.22 (m, 2H), 2.05 (s, 2H), 2.01-1.87 (m, 3H), 1.72-1.59 (m, 2H).

Synthesis of 86-E and 86-F: Prepared according to the procedure described for the preparation of compounds 1-F and 1-G.

Compound 86-E: (180 mg, 25% yield). MS(ESI) m/z 397.2 (M+H)⁺. ¹H NMR (600 MHz, Chloroform-d) δ 7.63 (s, 1H), 7.37-7.28 (m, 3H), 7.27 (s, 2H), 4.68-4.53 (m, 2H), 3.55-3.45 (m, 1H), 3.41-3.31 (m, 1H), 3.27 (d, J=15.2 Hz, 2H), 2.95-2.88 (m, 1H), 2.33-2.24 (m, 1H), 2.20-2.04 (m, 2H), 2.01-1.95 (m, 1H), 1.95-1.84 (m, 5H), 1.76-1.68 (m, 1H), 1.55-1.46 (m, 2H).

Compound 86-F: (365 mg, 51% yield). MS(ESI) m/z 397.2 (M+H)⁺. ¹H NMR (600 MHz, Chloroform-d) δ 8.76 (d, J=49.2 Hz, 1H), 7.42-7.29 (m, 4H), 7.22-7.11 (m, 1H), 6.52 (d, J=25.9 Hz, 1H), 4.96-4.86 (m, 1H), 4.80 (s, 1H), 4.64 (s, 1H), 3.55-3.45 (m, 1H), 3.30-3.17 (m, 1H), 2.38 (s, 2H), 2.13-1.95 (m, 3H), 1.78-1.53 (m, 4H), 0.97 (t, J=7.2 Hz, 2H), 0.78 (t, J=6.9 Hz, 2H).

### Step 2: Synthesis of 1-benzyl-7′-cyclopentyl-2′-((5-(piperazin-1-yl)pyridin-2-yl)amino)-5′,7′-dihydrospiro[piperidine-3,6′-pyrrolo[2,3-d]pyrimidin]-2-one (Compound C27)

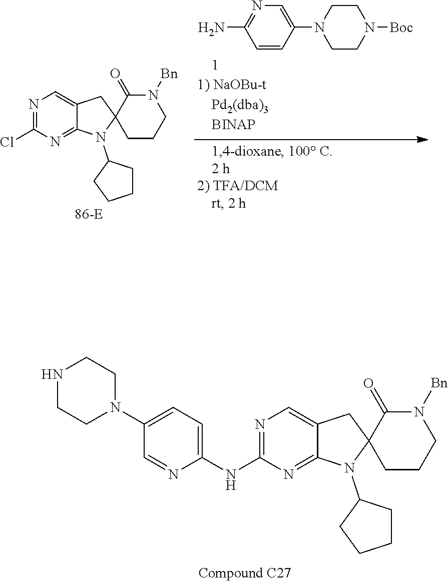 [SMILES: CC(C)(C)OC(=O)N1CCN(c2ccc(N)nc2)CC1 ; O=C1N(Cc2ccccc2)CCCC12Cc1cnc(Cl)nc1N2C1CCCC1 ; O=C1N(Cc2ccccc2)CCCC12Cc1cnc(Nc3ccc(N4CCNCC4)cn3)nc1N2C1CCCC1]

Compound C27 was synthesized following the general procedure A, 72 mg (40% yield). MS(ESI) m/z 539.3 (M+H)⁺. ¹H NMR (600 MHz, DMSO-d₆) δ 8.84 (bs, 2H), 8.01 (s, 1H), 7.69 (s, 2H), 7.40-7.33 (m, 2H), 7.33-7.24 (m, 4H), 4.58-4.50 (m, 2H), 3.72 (p, J=8.8 Hz, 1H), 3.40-3.35 (m, 5H), 3.30-3.26 (m, 5H), 3.14 (d, J=7.4 Hz, 2H), 2.40-2.31 (m, 1H), 2.27-2.18 (m, 1H), 2.09-1.98 (m, 2H), 1.95-1.83 (m, 4H), 1.83-1.72 (m, 2H), 1.59-1.47 (m, 2H).

### Example 87: Compound C28

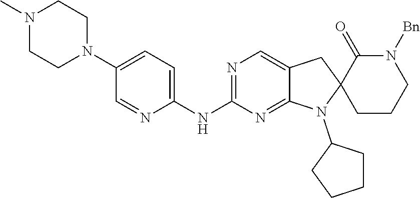 [SMILES: CN1CCN(c2ccc(Nc3ncc4c(n3)N(C3CCCC3)C3(CCCN(Cc5ccccc5)C3=O)C4)nc2)CC1]

### Synthesis of 1-benzyl-7′-cyclopentyl-2′-((5-(4-methylpiperazin-1-yl)pyridin-2-yl)amino)-5′,7′-dihydrospiro[piperidine-3,6′-pyrrolo[2,3-d]pyrimidin]-2-one (Compound C28)

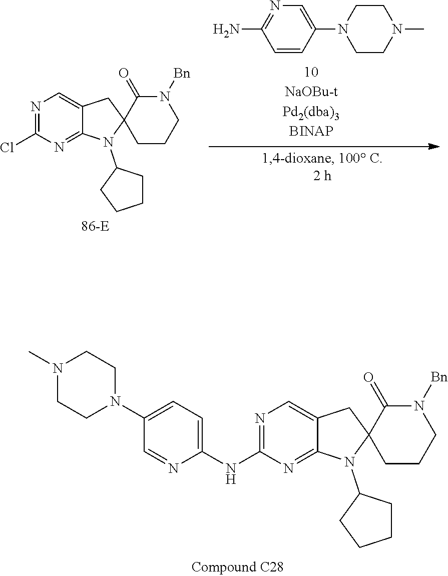 [SMILES: CN1CCN(c2ccc(N)nc2)CC1 ; O=C1N(Cc2ccccc2)CCCC12Cc1cnc(Cl)nc1N2C1CCCC1 ; CN1CCN(c2ccc(Nc3ncc4c(n3)N(C3CCCC3)C3(CCCN(Cc5ccccc5)C3=O)C4)nc2)CC1]

Compound C28 was synthesized following the general procedure B, 17 mg (9% yield). MS(ESI) m/z 553.3 (M+H)⁺. ¹H NMR (600 MHz, DMSO-d₆) δ 8.40 (s, 1H), 8.07 (d, J=9.1 Hz, 1H), 7.91 (d, J=2.9 Hz, 1H), 7.58 (s, 1H), 7.39-7.32 (m, 3H), 7.32-7.23 (m, 3H), 4.53 (s, 2H), 3.54-3.44 (m, 1H), 3.22 (d, J=10.5 Hz, 1H), 3.13-3.06 (m, 4H), 3.06-2.94 (m, 2H), 2.48-2.42 (m, 5H), 2.37-2.29 (m, 1H), 2.22 (s, 3H), 2.21-2.12 (m, 2H), 1.89-1.81 (m, 5H), 1.73-1.62 (m, 2H), 1.55-1.42 (m, 2H).

### Example 88: Compound C40

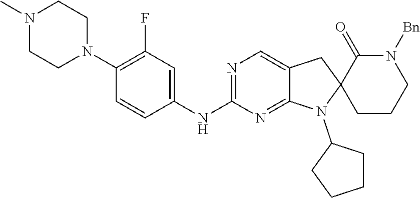 [SMILES: CN1CCN(c2ccc(Nc3ncc4c(n3)N(C3CCCC3)C3(CCCN(Cc5ccccc5)C3=O)C4)cc2F)CC1]

### Step 1: Synthesis of N-benzyl-3-(phenylselanyl)propan-1-amine (88C)

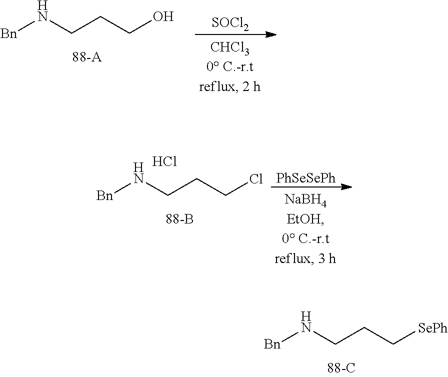

Synthesis of 88-B: Prepared according to the procedure described for the preparation of compound 84-B. (25 g, 94% yield) as a white solid. MS(ESI) m/z 184.1 [M+H]⁺. ¹H NMR (600 MHz, DMSO-d₆) δ 9.51 (s, 1H), 7.58 (d, J=6.8 Hz, 2H), 7.43 (q, J=8.2, 7.2 Hz, 3H), 4.14 (s, 2H), 3.75 (t, J=6.4 Hz, 2H), 3.00 (t, J=7.3 Hz, 2H), 2.22-2.11 (m, 2H).

Synthesis of 65: Prepared according to the procedure described for the preparation of compound 1-B. (20 g, 73% yield) as a white solid. MS(ESI) m/z 305.1 [M+H]⁺. ¹H NMR (600 MHz, Chloroform-d) δ 7.51-7.47 (m, 2H), 7.34-7.27 (m, 4H), 7.27-7.20 (m, 4H), 3.75 (s, 2H), 2.97 (t, J=7.3 Hz, 2H), 2.73 (t, J=6.9 Hz, 2H), 1.90 (p, J=7.1 Hz, 2H).

### Step 2: Synthesis of 1-benzyl-7′-cyclopentyl-2′-(methylthio)-5′,7′-dihydrospiro [piperidine-3,6′-pyrrolo[2,3-d]pyrimidin]-2-one (88-E)

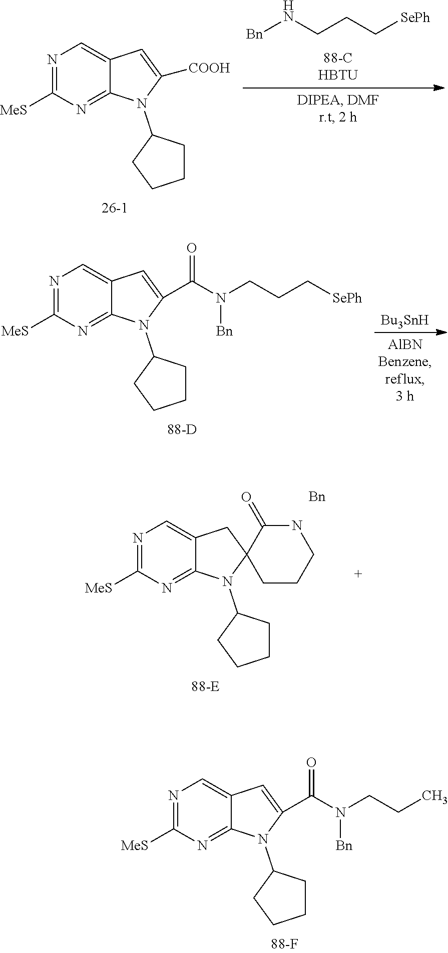

Synthesis of 88-D: Prepared according to the procedure described for the preparation of compound 1-D. (3.6 g, 89% yield) as a gummy liquid. MS(ESI) m/z 565.2 [M+H]⁺. ¹H NMR (600 MHz, Chloroform-d) δ 8.78-8.59 (m, 1H), 7.48 (s, 1H), 7.39-7.28 (m, 5H), 7.17 (d, J=45.3 Hz, 4H), 6.50-6.32 (m, 1H), 4.87-4.61 (m, 3H), 3.61 (s, 1H), 3.39 (s, 1H), 2.92 (s, 1H), 2.67 (s, 1H), 2.60 (s, 3H), 2.47 (s, 2H), 2.14-1.83 (m, 6H), 1.66 (s, 2H).

Synthesis of 88-E and 88-F: Prepared according to the procedure described for the preparation of compounds 1-F and 1-G.

Compound 88-E: (0.75 g, 36% yield). MS(ESI) m/z 409.2 [M+H]⁺. ¹H NMR (600 MHz, Chloroform-d) δ 7.65 (s, 1H), 7.36-7.27 (m, 5H), 4.66-4.57 (m, 2H), 3.49-3.31 (m, 2H), 3.30-3.21 (m, 2H), 2.95-2.85 (m, 1H), 2.48 (s, 3H), 2.47-2.40 (m, 1H), 2.29-2.18 (m, 1H), 2.17-2.06 (m, 1H), 1.92-1.84 (m, 5H), 1.81-1.71 (m, 2H), 1.56-1.45 (m, 2H).

Compound 88-F: (1.1 g, 52% yield). MS(ESI) m/z 409.2 [M+H]⁺. ¹H NMR (600 MHz, Chloroform-d) δ 8.82-8.63 (m, 1H), 7.46-7.28 (m, 4H), 7.23-7.12 (m, 1H), 6.52-6.40 (m, 1H), 4.93-4.65 (m, 3H), 3.49 (s, 1H), 3.26 (s, 1H), 2.60 (s, 3H), 2.55-2.44 (m, 3H), 2.41 (s, 1H), 2.12-1.98 (m, 5H), 1.79-1.63 (m, 4H).

### Step 3: Synthesis of 1-benzyl-7′-cyclopentyl-2′-(methylsulfonyl)-5′,7′-dihydrospiro [piperidine-3,6′-pyrrolo[2,3-d]pyrimidin]-2-one (88-G)

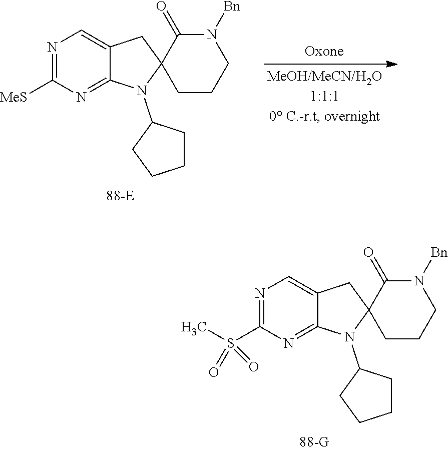 [SMILES: CSc1ncc2c(n1)N(C1CCCC1)C1(CCCN(Cc3ccccc3)C1=O)C2 ; CS(=O)(=O)c1ncc2c(n1)N(C1CCCC1)C1(CCCN(Cc3ccccc3)C1=O)C2]

Synthesis of 88-G: Prepared according to the procedure described for the preparation of compounds 84-G (581 mg, 77% yield). MS(ESI) m/z 441.2 [M+H]⁺. ¹H NMR (600 MHz, Chloroform-d) δ 7.89 (s, 1H), 7.38-7.30 (m, 4H), 7.27 (s, 1H), 4.68-4.52 (m, 3H), 3.54-3.45 (m, 1H), 3.43-3.32 (m, 2H), 3.33-3.27 (m, 1H), 3.23 (s, 3H), 3.05-2.98 (m, 1H), 2.40-2.29 (m, 1H), 2.25-2.10 (m, 2H), 1.97-1.86 (m, 6H), 1.59-1.50 (m, 2H).

### Step 4: Synthesis of 1-benzyl-7′-cyclopentyl-2′-((3-fluoro-4-(4-methylpiperazin-1-yl)phenyl)amino)-5′,7′-dihydrospiro[piperidine-3,6′-pyrrolo[2,3-d]pyrimidin]-2-one (Compound C40)

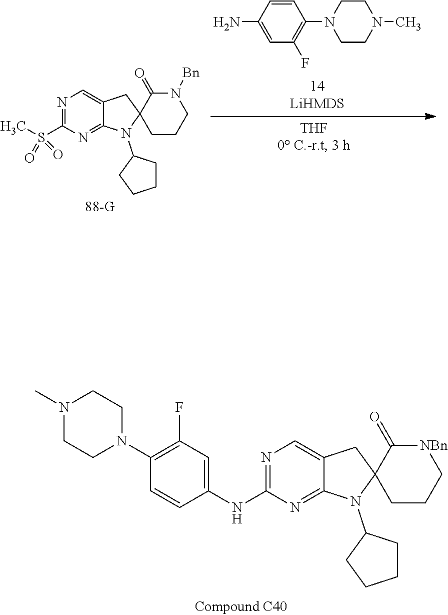 [SMILES: CN1CCN(c2ccc(N)cc2F)CC1 ; CS(=O)(=O)c1ncc2c(n1)N(C1CCCC1)C1(CCCN(Cc3ccccc3)C1=O)C2 ; CN1CCN(c2ccc(Nc3ncc4c(n3)N(C3CCCC3)C3(CCCN(Cc5ccccc5)C3=O)C4)cc2F)CC1]

Compound C40 was synthesized following the general procedure E, 19 mg (14% yield). MS(ESI) m/z 570.3 [M+H]⁺. ¹H NMR (600 MHz, Chloroform-d) δ 7.69 (dd, J=14.9, 2.3 Hz, 1H), 7.51 (s, 1H), 7.37-7.29 (m, 3H), 7.28 (s, 2H), 6.99 (dd, J=8.6, 2.0 Hz, 1H), 6.88 (t, J=9.1 Hz, 1H), 4.66-4.57 (m, 2H), 3.44-3.31 (m, 2H), 3.29-3.19 (m, 2H), 3.07 (s, 4H), 2.89 (d, J=15.5 Hz, 1H), 2.61 (s, 4H), 2.54-2.45 (m, 1H), 2.36 (s, 3H), 2.35-2.26 (m, 1H), 2.19-2.08 (m, 2H), 1.91 (s, 5H), 1.75-1.65 (m, 1H), 1.59-1.45 (m, 2H).

### Example 89: Compound C42

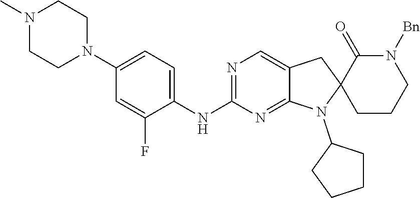 [SMILES: CN1CCN(c2ccc(Nc3ncc4c(n3)N(C3CCCC3)C3(CCCN(Cc5ccccc5)C3=O)C4)c(F)c2)CC1]

### Synthesis of 1-benzyl-7′-cyclopentyl-2′-((2-fluoro-4-(4-methylpiperazin-1-yl)phenyl)amino)-5′,7′-dihydrospiro[piperidine-3,6′-pyrrolo[2,3-d]pyrimidin]-2-one (Compound C42)

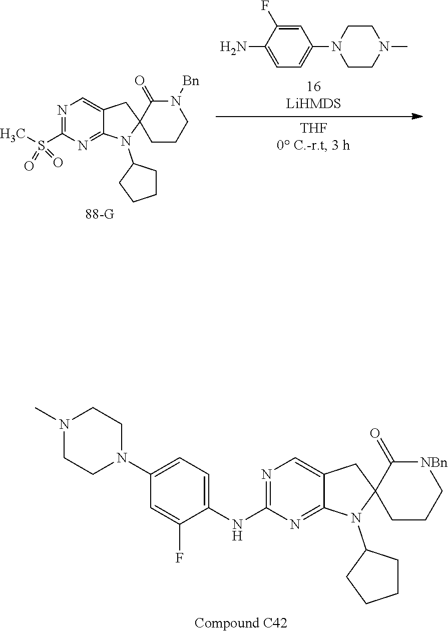 [SMILES: CN1CCN(c2ccc(N)c(F)c2)CC1 ; CS(=O)(=O)c1ncc2c(n1)N(C1CCCC1)C1(CCCN(Cc3ccccc3)C1=O)C2 ; CN1CCN(c2ccc(Nc3ncc4c(n3)N(C3CCCC3)C3(CCCN(Cc5ccccc5)C3=O)C4)c(F)c2)CC1]

Compound C42 was synthesized following the general procedure E, 6 mg (5% yield). MS(ESI) m/z 570.3 [M+H]⁺. ¹H NMR (400 MHz, Chloroform-d) δ 7.71 (d, J=14.9, 1H), 7.50 (s, 1H), 7.34-7.28 (m, 3H), 7.26-7.69 (m, 2H), 6.97-6.85 (m, 1H), 6.88 (t, J=9.1 Hz, 1H), 4.61-4.60 (m, 2H), 3.39-3.31 (m, 2H), 3.29-3.19 (m, 2H), 3.06 (s, 4H), 2.89 (d, J=15.5 Hz, 1H), 2.61 (s, 4H), 2.54-2.45 (m, 1H), 2.36 (s, 3H), 2.35-2.26 (m, 1H), 2.19-2.08 (m, 2H), 1.91 (s, 5H), 1.75-1.65 (m, 1H), 1.59-1.45 (m, 2H).

### Example 90: Compound C29

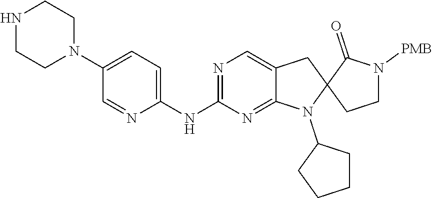

### Step 1: Synthesis of N-(4-methoxybenzyl)-2-(phenylselanyl)ethan-1-amine (90-E)

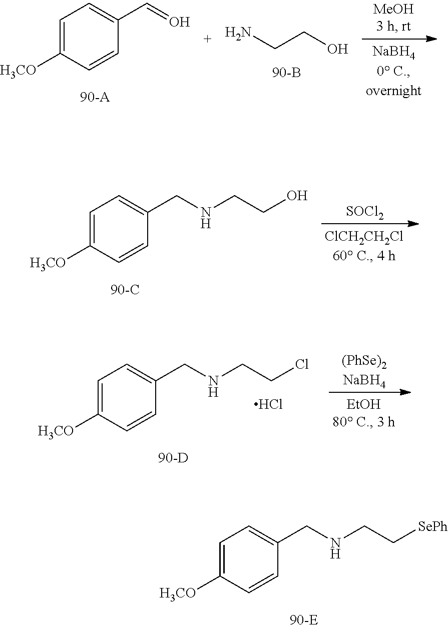

Synthesis of 90-C: (Ref: WO 2013072903): 4-Methoxybenzaldehyde 90-A (15 g, 110.1 mmol) was added to a stirring solution of ethanolamine 90-B (6.7 g, 110.1 mmol) in methanol (150 mL). The resulting solution was allowed to stir for 2 hours at room temperature. Sodium borohydride (6.2 g, 165.1 mmol) was added cautiously at 0° C. over 45 minutes. The reaction mixture was allowed to stir at room temperature for 18 hours. Thereafter the reaction mixture was concentrated under vacuum, taken up into DCM (200 mL), and washed consecutively with saturated sodium carbonate solution (100 mL), water (100 mL) and brine. The organic layer was separated, dried over sodium sulfate, filtered, and evaporated in vacuo to yield 90-C, which was used in the next step without any further purification (crude weight 19.5 g). MS(ESI) m/z 182.2 (M+H)⁺. ¹H NMR (600 MHz, Chloroform-d) δ 7.23 (d, J=8.6 Hz, 2H), 6.89-6.83 (m, 2H), 3.80 (s, 3H), 3.75 (s, 2H), 3.69-3.61 (m, 2H), 2.85-2.76 (m, 2H).

Synthesis of 90-D: (Ref: WO 2010065717): To compound 90-C (16 g, 88.28 mol) in 1,2-dichloroethane (400 mL) was added Thionyl chloride (9 mL, 114.6 mmol dropwise while maintaining the temperature of the batch between 20 and 30° C. Once the addition was complete, the batch was heated at 60° C. for 4 hours. The batch was cooled to room temperature and concentrated using a rotoevaporator. The concentrate was swapped once with MTBE (250 mL), slurried in MTBE (250 mL), filtered, and dried to give compound 90-D (18 g, 87%). MS(ESI) m/z 200.1 (M+H)⁺. ¹H NMR (600 MHz, DMSO-d₆) δ 9.34 (s, 2H), 7.47 (d, J=8.7 Hz, 2H), 6.99 (d, J=8.7 Hz, 2H), 4.12 (s, 2H), 3.91 (t, J=6.3 Hz, 2H), 3.77 (s, 3H), 3.24 (s, 2H).

Synthesis of 90-E: Prepared according to the procedure described for the preparation of compound 1-B. (11 g, 81% yield). MS(ESI) m/z 322.1 (M+H)⁺. ¹H NMR (600 MHz, Chloroform-d) δ 7.48-7.45 (m, 2H), 7.25-7.22 (m, 3H), 7.20 (d, J=8.6 Hz, 2H), 6.85 (d, J=8.6 Hz, 2H), 3.80 (s, 3H), 3.72 (s, 2H), 3.06 (t, J=6.6 Hz, 2H), 2.88 (t, J=6.6 Hz, 2H).

### Step 2: Synthesis of 2′-chloro-7′-cyclopentyl-1-(4-methoxybenzyl)-5′,7′-dihydrospiro[pyrrolidine-3,6′-pyrrolo[2,3-d]pyrimidin]-2-one (90-G)

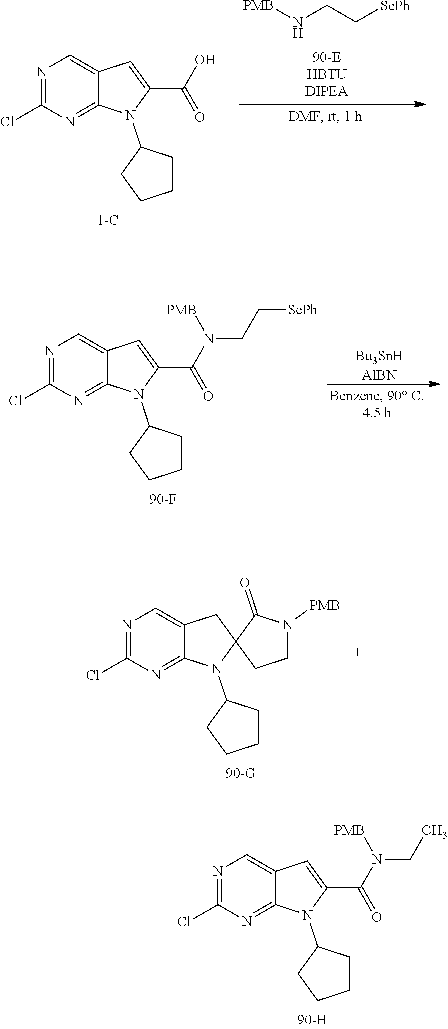

Synthesis of 32: Prepared according to the procedure described for the preparation of compound 1-D. (2.3 g, 90% yield). MS(ESI) m/z 569.1 (M+H)⁺. ¹H NMR (600 MHz, Chloroform-d) δ 8.75 (s, 0.5H), 8.66 (s, 0.5H), 7.48 (s, 1H), 7.27 (s, 1H), 7.20 (s, 2H), 7.11-7.04 (m, 1H), 6.94 (s, 2H), 6.85 (dd, J=19.0, 7.4 Hz, 2H), 6.42 (d, J=101.3 Hz, 1H), 4.98-4.86 (m, 1H), 4.68 (s, 1H), 4.56 (s, 1H), 3.85-3.77 (m, 3H), 3.75 (d, J=11.4 Hz, 1H), 3.44 (s, 1H), 3.18 (s, 1H), 2.86-2.75 (m, 1H), 2.41 (s, 1H), 2.27 (s, 1H), 2.09 (s, 2H), 1.99 (s, 2H), 1.70 (s, 1H), 1.62 (s, 1H).

Synthesis of 90-G and 90-H: Prepared according to the procedure described for the preparation of compound 1-F and 1-G.

Compound 90-G: 730 mg (43% yield). MS(ESI) m/z 413.2 (M+H)⁺. ¹H NMR (600 MHz, Chloroform-d) δ 7.67 (s, 1H), 7.18 (d, J=8.6 Hz, 2H), 6.92-6.84 (m, 2H), 4.45 (d, 2H), 3.81 (s, 3H), 3.48-3.38 (m, 1H), 3.31-3.18 (m, 3H), 2.90-2.83 (m, 1H), 2.35-2.25 (m, 2H), 2.23-2.09 (m, 2H), 1.97-1.83 (m, 3H), 1.76-1.68 (m, 1H), 1.55-1.45 (m, 2H).

Compound 90-H: 175 mg (11% yield). MS(ESI) m/z 413.2 (M+H)⁺. ¹H NMR (600 MHz, Chloroform-d) δ 8.76 (d, J=33.5 Hz, 1H), 7.31 (d, J=7.5 Hz, 1H), 7.09 (d, J=6.9 Hz, 1H), 6.90 (d, J=8.6 Hz, 2H), 6.53 (d, J=14.3 Hz, 1H), 5.00-4.80 (m, 1H), 4.73 (s, 1H), 4.55 (s, 1H), 3.82 (s, 4H), 3.64-3.51 (m, 1H), 3.27 (d, J=7.7 Hz, 1H), 2.45-2.25 (m, 2H), 2.17-1.95 (m, 5H), 1.28-1.19 (m, 2H), 1.15 (s, 2H).

### Step 3: Synthesis of 7′-cyclopentyl-1-(4-methoxybenzyl)-2′-((5-(piperazin-1-yl)pyridin-2-yl)amino)-5′,7′-dihydrospiro[pyrrolidine-3,6′-pyrrolo[2,3-d]pyrimidin]-2-one (Compound C29)

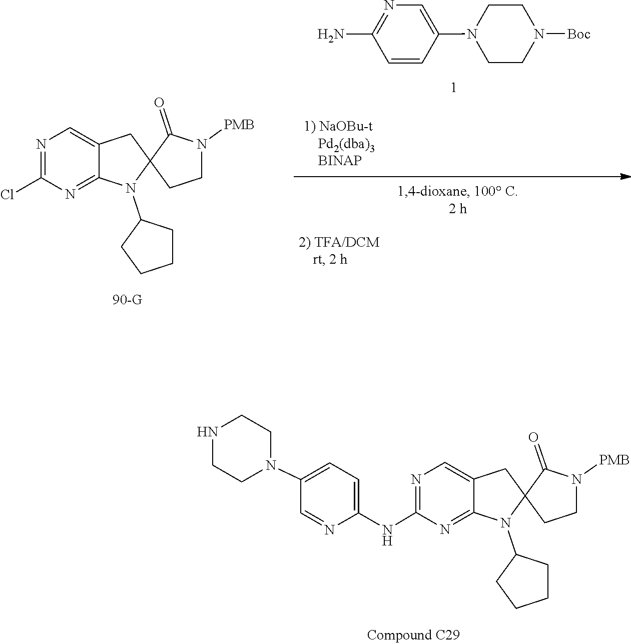

Compound C29 was synthesized following the general procedure A, 4 mg (2% yield). MS(ESI) m/z 555.3 (M+H)⁺. ¹H NMR (600 MHz, DMSO-d₆) δ 8.83 (bs, 2H), 8.01 (d, J=2.7 Hz, 1H), 7.72 (s, 1H), 7.68 (d, J=7.3 Hz, 1H), 7.32 (s, 1H), 7.21 (d, J=8.5 Hz, 2H), 6.94 (d, J=8.6 Hz, 2H), 4.51-4.32 (m, 2H), 3.75 (s, 3H), 3.58-3.51 (m, 1H), 3.38 (s, 4H), 3.29-3.22 (m, 5H), 3.17-3.06 (m, 2H), 2.46-2.37 (m, 2H), 2.30-2.23 (m, 1H), 2.24-2.18 (m, 1H), 2.18-2.10 (m, 1H), 1.87 (s, 2H), 1.84-1.77 (m, 1H), 1.77-1.70 (m, 1H), 1.56-1.44 (m, 2H).

### Example 91: Compound C30

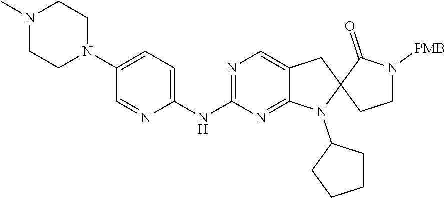

### Synthesis of 7′-cyclopentyl-1-(4-methoxybenzyl)-2′-((5-(4-methylpiperazin-1-yl)pyridin-2-yl)amino)-5′,7′-dihydrospiro[pyrrolidine-3,6′-pyrrolo[2,3-d]pyrimidin]-2-one (Compound C30)

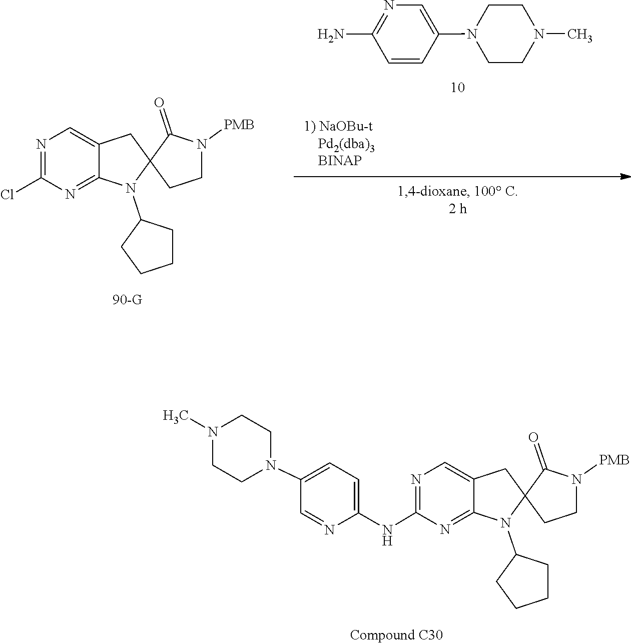

Compound C30 was synthesized following the general procedure B, 10 mg (5% yield). MS(ESI) m/z 569.3 (M+H)⁺. ¹H NMR (600 MHz, DMSO-d₆) δ 8.50 (s, 1H), 8.04 (d, J=8.0 Hz, 1H), 7.92 (s, 1H), 7.62 (s, 1H), 7.35 (d, J=7.4 Hz, 1H), 7.19 (d, J=6.6 Hz, 2H), 6.93 (d, J=6.5 Hz, 2H), 4.47-4.29 (m, 2H), 3.75 (s, 3H), 3.28-3.15 (m, 3H), 3.08 (s, 4H), 2.96 (s, 2H), 2.46 (s, 4H), 2.29 (s, 2H), 2.22 (s, 4H), 2.15 (s, 1H), 1.84 (s, 2H), 1.68 (s, 2H), 1.45 (s, 2H).

### Example 92: Compound C31

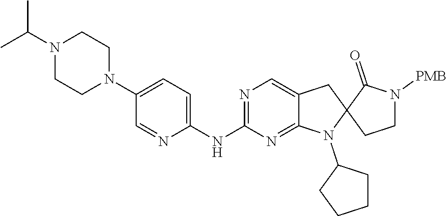

### Synthesis of 7′-cyclopentyl-2′-((5-(4-isopropylpiperazin-1-yl)pyridin-2-yl)amino)-1-(4-methoxybenzyl)-5′,7′-dihydrospiro[pyrrolidine-3,6′-pyrrolo[2,3-d]pyrimidin]-2-one (Compound C31)

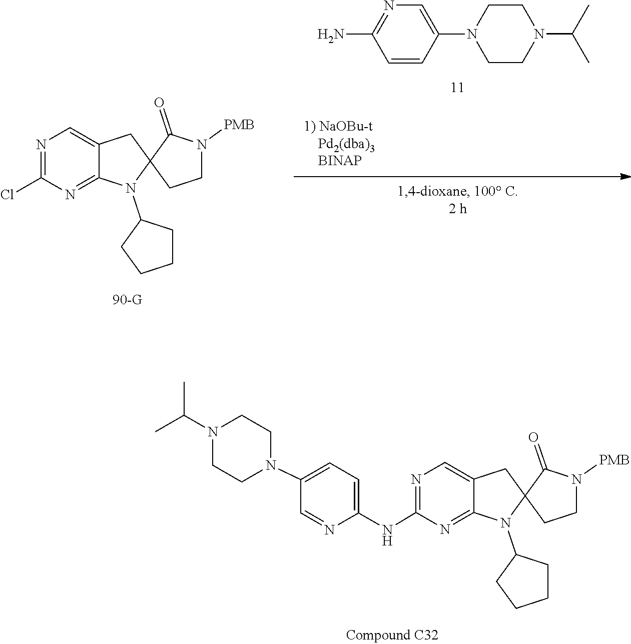

Compound C31 was synthesized following the general procedure B, 31 mg (14% yield). MS(ESI) m/z 597.3 (M+H)⁺. ¹H NMR (600 MHz, DMSO-d₆) δ 8.48 (s, 1H), 8.04 (d, J=9.1 Hz, 1H), 7.91 (d, J=2.9 Hz, 1H), 7.62 (s, 1H), 7.33 (dd, J=9.1, 3.0 Hz, 1H), 7.19 (d, J=8.6 Hz, 2H), 6.93 (d, J=8.6 Hz, 2H), 4.47-4.30 (m, 2H), 3.75 (s, 3H), 3.45-3.34 (m, 2H), 3.29-3.16 (m, 2H), 3.10-3.03 (m, 3H), 2.95 (s, 2H), 2.72-2.63 (m, 1H), 2.60-2.56 (m, 3H), 2.35-2.18 (m, 3H), 2.18-2.11 (m, 1H), 1.83 (s, 2H), 1.73-1.62 (m, 2H), 1.52-1.39 (m, 2H), 1.00 (d, J=6.5 Hz, 6H).

### Example 93: Compound C12

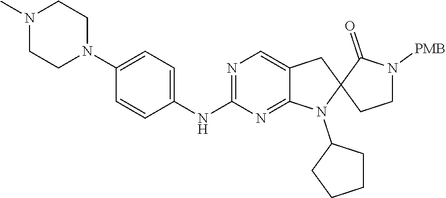

### Synthesis of 7′-cyclopentyl-1-(4-methoxybenzyl)-2′-((4-(4-methylpiperazin-1-yl)phenyl)amino)-5′,7′-dihydrospiro[pyrrolidine-3,6′-pyrrolo[2,3-d]pyrimidin]-2-one (Compound C12)

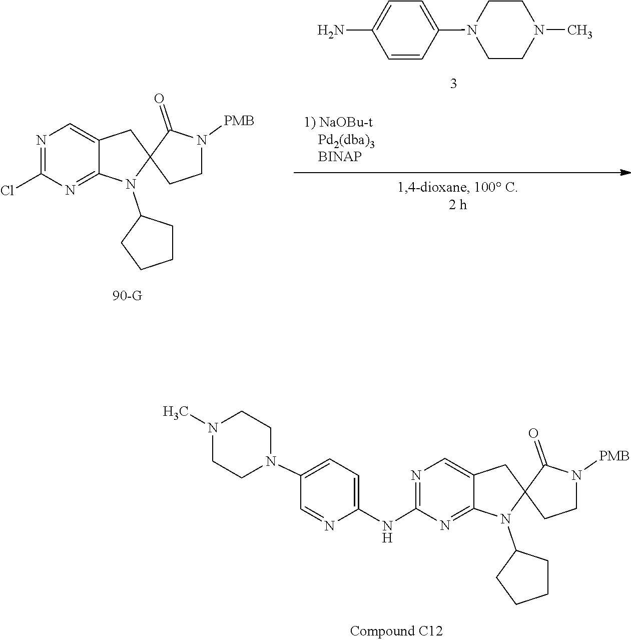

Compound C12 was synthesized following the general procedure B, 7 mg (5% yield). MS(ESI) m/z 568.3 (M+H)⁺.

### Example 94: Compound C16

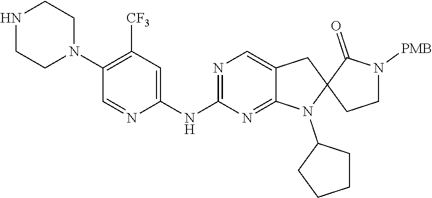

### Synthesis of 7′-cyclopentyl-1-(4-methoxybenzyl)-2′-((5-(piperazin-1-yl)-4-(trifluoromethyl)pyridin-2-yl)amino)-5′,7′-dihydrospiro[pyrrolidine-3,6′-pyrrolo[2,3-d]pyrimidin]-2-one (Compound C16)

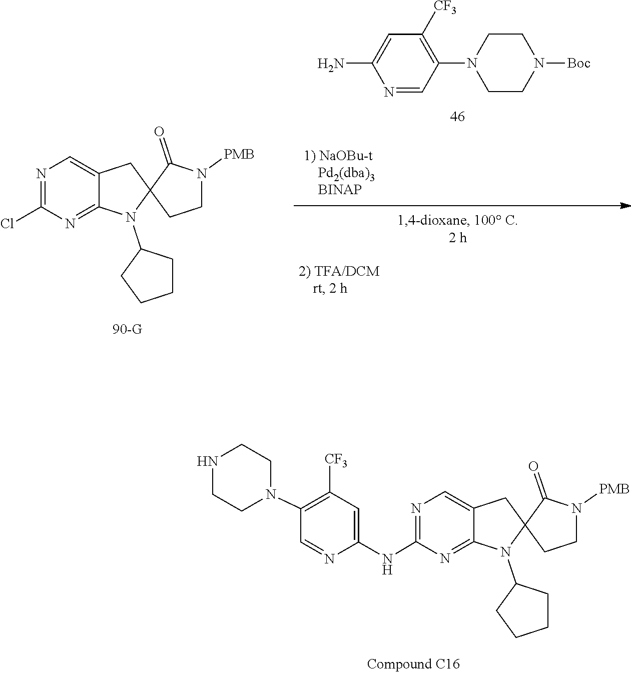

Compound C16 was synthesized following the general procedure A, 23 mg (15% yield). MS(ESI) m/z 623.3 (M+H)⁺.

### Example 95: Compound C32

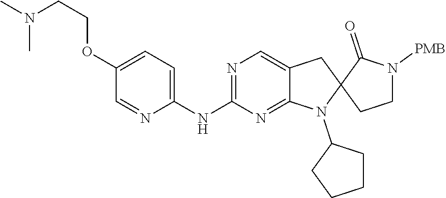

### Synthesis of 7′-cyclopentyl-2′-((5-(2-(dimethylamino)ethoxy)pyridin-2-yl)amino)-1-(4-methoxybenzyl)-5′,7′-dihydrospiro[pyrrolidine-3,6′-pyrrolo[2,3-d]pyrimidin]-2-one (Compound C32)

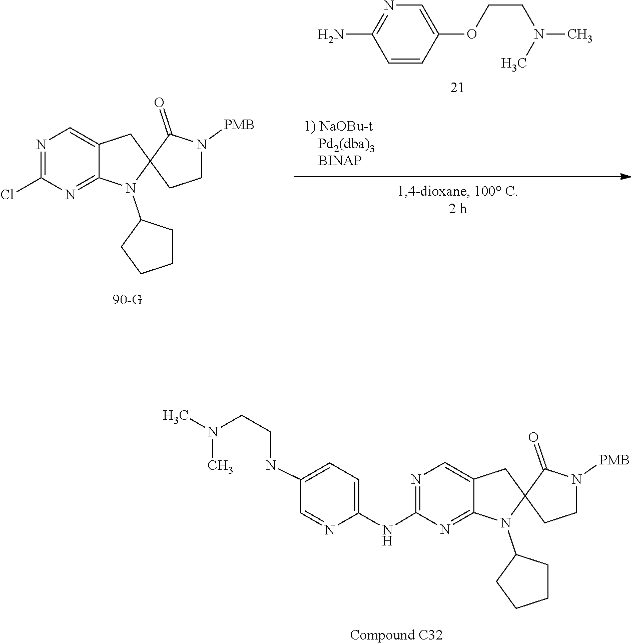

Compound C32 was synthesized following the general procedure B, 43 mg (21% yield). MS(ESI) m/z 558.3 (M+H)⁺.

### Example 96: Compound C98

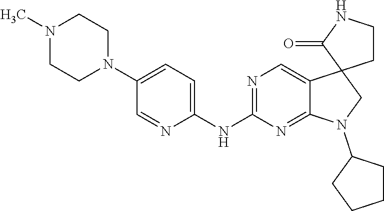 [SMILES: CN1CCN(c2ccc(Nc3ncc4c(n3)N(C3CCCC3)CC43CCNC3=O)nc2)CC1]

### Synthesis of tert-butyl 2′-chloro-7′-cyclopentyl-2-oxo-6′,7′-dihydrospiro[pyrrolidine-3,5′-pyrrolo[2,3-d]pyrimidine]-1-carboxylate (96-H)

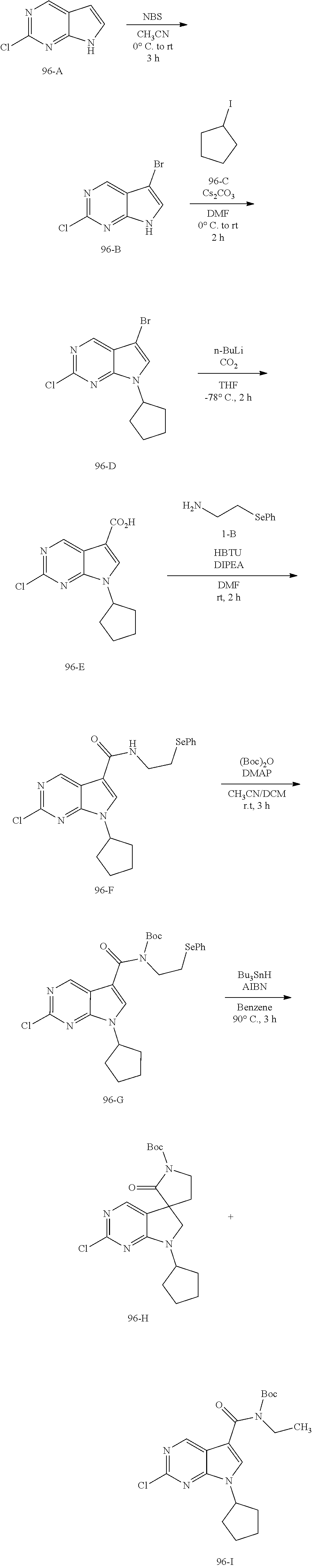

Synthesis of 96-B: (Ref: WO 2012137089): N-Bromo succinimide (32 g, 180 mmol) was added to 96-A (23 g, 150 mmol) in acetonitrile (800 mL) at 12° C. The mixture was stirred at room temperature for 3 hour then sodium metabisulphite (30 g in 200 mL of water) was added. The mixture was stirred for 1 hour then filtered to afford the title compound 96-B as an off-white solid (29 g, 84% yield). MS(ESI) m/z 232.0 (M+H)⁺. ¹H NMR (600 MHz, Chloroform-d) δ 9.56 (s, 1H), 8.82 (s, 1H), 7.37 (s, 1H).

Synthesis of 96-D: (Ref: WO 2012137089): To a mixture of 96-B (3.5 g, 15.1 mol), Cs₂CO3(7.4 g, 22.6 mol) in DMF (50 mL) was added iodo cyclopentane (4.5 g, 22.7 mol). The resulting mixture was stirred at RT for 3 h. The reaction mixture was then poured onto saturated NH₄Cl solution and the solid was filtered and dried to give the desired compound 96-D (2.8 g, 62% yield). MS(ESI) m/z 300.1 (M+H)⁺. ¹H NMR (600 MHz, Chloroform-d) δ 8.72 (s, 1H), 7.29 (s, 1H), 5.27-5.15 (m, 1H), 2.32-2.21 (m, 2H), 1.96-1.88 (m, 2H), 1.87-1.74 (m, 4H).

Synthesis of 96-E: (Ref: WO 2011018894): To a stirred solution of 96-D (2.8 g, 9.3 mmol) in tetrahydrofuran (50 mL) was added 1.6 M n-butyllithium in hexane solution (7 mL, 11.2 mmol) slowly at −78° C. After stirring at −78° C. for 1 hr, solid CO₂ was added to the mixture. The resulting was warmed to room temperature gradually and quenched with 1 M HCl aqueous solution. The mixture was extracted with dichloromethane/methanol (100 mL×4), and the combined organic fraction was dried over sodium sulfate. After removal of the solvent by evaporation, the crude residue was used as such for next step. MS(ESI) m/z 266.1 (M+H)⁺.

Synthesis of 96-F: Prepared according to the procedure described for the preparation of Compound 1-D. (1.3 g, 31% yield, over two steps). MS(ESI) m/z 449.1 (M+H)⁺. ¹H NMR (600 MHz, Chloroform-d) δ 9.18 (s, 1H), 7.61 (s, 1H), 7.58-7.51 (m, 2H), 7.25-7.20 (m, 3H), 6.23 (s, 1H), 5.20 (p, J=7.6 Hz, 1H), 3.77 (q, J=6.1 Hz, 2H), 3.18 (t, J=6.3 Hz, 2H), 2.34-2.23 (m, 2H), 1.98-1.88 (m, 2H), 1.89-1.76 (m, 4H).

Synthesis of 96-G: (Ref: Putey et al., J. Med. Chem. 52(19): 5916-5925 (2009)): A solution of amide 96-F (1.3 g, 2.9 mmol), Boc₂O (1.2 g, 5.8 mmol), and a catalytic amount of DMAP (177 mg, 1.45 mmol) in MeCN/DCM (30 mL/30 mL) was stirred overnight at room temperature for 2 h. After evaporation of the solvent, the residue was partitioned between DCM) and H₂O. The two phases were separated, and the aqueous phase was extracted with DCM (2×25 mL). The combined organic phases were dried over Na₂SO₄ and concentrated in vacuo. The crude residue was purified by flash chromatography (Hexane/EtOAc 4:1) to give 96-G (1.1 g, 70%). MS(ESI) m/z 549.1 (M+H)⁺. ¹H NMR (600 MHz, Chloroform-d) δ 8.96 (s, 1H), 7.81 (s, 1H), 7.57-7.53 (m, 2H), 7.30-7.26 (m, 2H), 7.26-7.23 (m, 1H), 5.19 (p, J=7.3 Hz, 1H), 4.12-4.04 (m, 2H), 3.24-3.15 (m, 2H), 2.36-2.24 (m, 2H), 1.95-1.88 (m, 2H), 1.88-1.77 (m, 4H).

Synthesis of 96-H and 96-I: Prepared according to the procedure described for the preparation of Compounds 1-F and 1-G.

Compound 96-H: (185 mg, 24% yield). MS(ESI) m/z 393.2 (M+H)⁺. ¹H NMR (600 MHz, Chloroform-d) δ 8.04 (d, J=1.6 Hz, 1H), 4.40-4.34 (m, 1H), 4.28-4.23 (m, 1H), 4.15-4.07 (m, 1H), 3.78-3.70 (m, 1H), 2.20-2.14 (m, 1H), 2.07-1.92 (m, 2H), 1.92-1.79 (m, 3H), 1.79-1.69 (m, 1H), 1.66-1.62 (m, 2H), 1.53 (s, 9H).

Compound 96-I: (364 mg, 47% yield). MS(ESI) m/z 393.2 (M+H)⁺. ¹H NMR (600 MHz, Chloroform-d) δ 8.93 (s, 1H), 7.80 (s, 1H), 5.26-5.16 (m, 1H), 3.85 (q, J=7.0 Hz, 2H), 2.33-2.25 (m, 2H), 1.96-1.89 (m, 2H), 1.89-1.77 (m, 4H), 1.38 (s, 9H), 1.35-1.28 (m, 4H).

### Step 3: Synthesis of 7′-cyclopentyl-2′-((5-(4-methylpiperazin-1-yl)pyridin-2-yl)amino)-6′,7′-dihydrospiro[pyrrolidine-3,5′-pyrrolo[2,3-d]pyrimidin]-2-one (Compound C98)

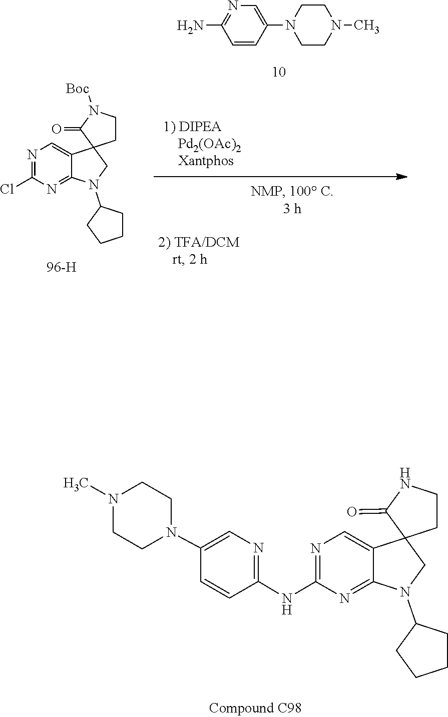 [SMILES: CN1CCN(c2ccc(N)nc2)CC1 ; CC(C)(C)OC(=O)N1CCC2(CN(C3CCCC3)c3nc(Cl)ncc32)C1=O ; CN1CCN(c2ccc(Nc3ncc4c(n3)N(C3CCCC3)CC43CCNC3=O)nc2)CC1]

Compound C98 was synthesized following the general procedure I, 2 mg. MS(ESI) m/z 449.3 (M+H)⁺.

### Example 97: Compound C101

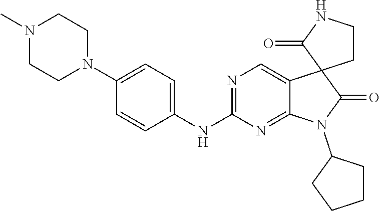 [SMILES: CN1CCN(c2ccc(Nc3ncc4c(n3)N(C3CCCC3)C(=O)C43CCNC3=O)cc2)CC1]

Step 1: Synthesis of tert-butyl 2′-chloro-7′-cyclopentyl-2,6′-dioxo-6′,7′-dihydrospiro[pyrrolidine-3,5′-pyrrolo[2,3-d]pyrimidine]-1-carboxylate (97-F)

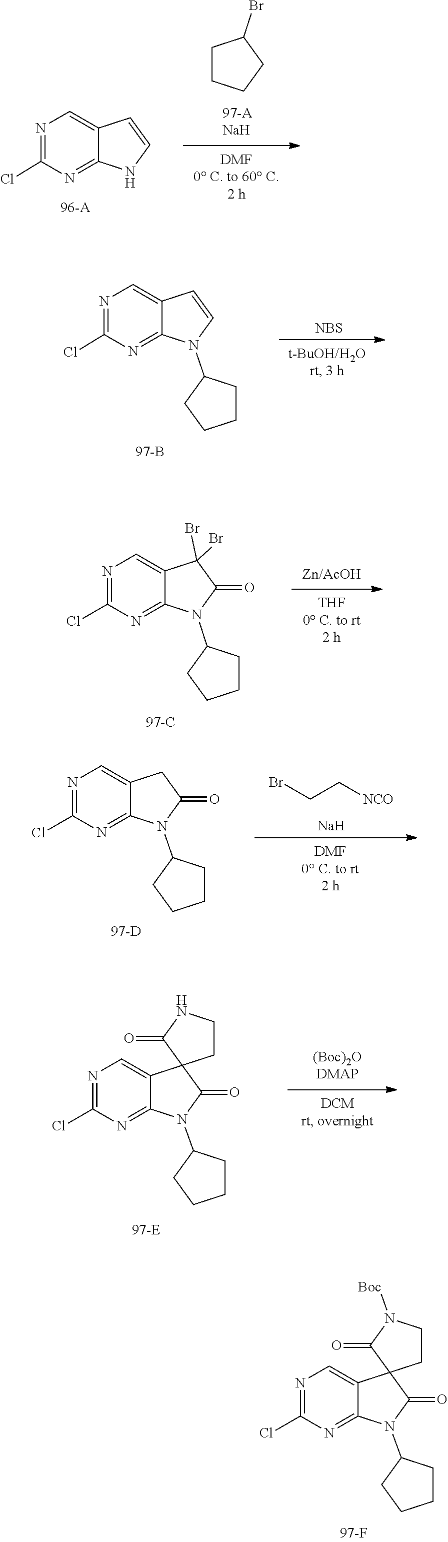

Synthesis of 97-B: (Ref: WO 2005047289): NaH (10.2 g, 244.5 mmol) was added to a solution of 4-Chloro-7H-pyrrolo[2,3-d]pyrimidine 96-A (25 g, 163 mmol) in DMF (250 mL) at 0° C. The resulting mixture was stirred at 0° C. for 30 min, and then warmed to room temperature. At this time, cyclopentylbromide 97-A (50 g, 326 mmol) was added and the reaction was heated to 60° C. After 2 h, the reaction was cooled to 0° C. and quenched slowly with water. The aqueous layer was extracted with EtOAc, the combined organic layers were washed with water and brine, dried over Na₂ SO₄ and concentrated. Purification by flash column chromatography (Hexanes/Ethyl acetate 9:1) afforded 97-B (33 g, 75% yield). MS(ESI) m/z 222.1 (M+H)⁺. ¹H NMR (600 MHz, Chloroform-d) δ 8.77 (s, 1H), 7.28 (d, J=3.7 Hz, 1H), 6.56 (d, J=3.6 Hz, 1H), 5.28-5.15 (m, 1H), 2.31-2.21 (m, 2H), 1.96-1.88 (m, 2H), 1.88-1.74 (m, 4H).

Synthesis of 97-C: (Ref: WO 2016171755): To a solution of 97-B (33 g, 149.2 mmol) in t-BuOH/H₂O (300 mL/90 mL) was added NBS (80 g, 447.6 mmol) and stirred for 3 h at room temperature. The reaction mixture was concentrated and dissolved in EtOAc. The EtOAc layer was washed with NaHCO₃, brine, dried over sodium sulfate and concentrated to give 97-C (64 g) which was used as such for the next step. MS(ESI) m/z 395.2 (M+H)⁺.

Synthesis of 97-D: (Ref: Putey et al., J. Med. Chem. 52(19): 5916-5925 (2009)): To a solution of 97-C (64 g, 149.2) in THF (300 mL), was added acetic acid (600 mL) at 0° C. and followed by Zn dust (39 g, 597 mmol). The resultant mixture was stirred at 0° C. for 5 min., then warmed to room temperature and stirred for 1 h. The reaction mixture was filtered through a pad of Celite and washed with EtOAc. The filtrate was concentrated and purified by ISCO (hexane:ethylacetate 0 to 40%) to give 97-D (21.2 g, 60%, over two steps). MS(ESI) m/z 238.1 (M+H)⁺. ¹H NMR (600 MHz, Chloroform-d) δ 8.15 (s, 1H), 4.84-4.70 (m, 1H), 3.53 (s, 2H), 2.22-2.11 (m, 2H), 2.04-1.95 (m, 2H), 1.95-1.85 (m, 2H), 1.70-1.60 (m, 2H).

Synthesis of 97-E: (Ref: Crosignani et al., J. Med. Chem. 51(7): 2227-2243 (2008)): NaH (262 mg, 6.3 mmol) was added to a solution of 97-D (1 g, 4.2 mmol) in DMF (20 mL) at 0° C. The resulting mixture was stirred at RT 30 min. At this time, 2-bromoethyl isocyanate (0.38 mL, 4.2 mmol) was added at RT and the reaction was stirred for 1 h. The reaction mixture was then diluted with ethyl acetate, washed with water and brine, and concentrated. Purification by flash column chromatography (DCM/MeOH 98:2) afforded the title compound 97-E (800 mg, 62% yield). MS(ESI) m/z 307.1 (M+H)⁺. ¹H NMR (600 MHz, Chloroform-d) δ 8.53 (s, 1H), 8.20 (s, 1H), 4.98-4.88 (m, 1H), 4.85-4.76 (m, 2H), 4.00-3.93 (m, 2H), 2.30-2.20 (m, 2H), 2.05-1.97 (m, 2H), 1.96-1.89 (m, 2H), 1.74-1.59 (m, 2H).

Synthesis of 97-F: (Ref: Putey et al., J. Med. Chem. 52(19): 5916-5925 (2009)): A solution of 97-E (400 mg, 1.3 mmol), Boc₂O (600 mg, 2.6 mmol), and a catalytic amount of DMAP (48 mg, 0.39 mmol) in DCM (20 mL) was stirred overnight at room temperature. After evaporation of the solvent, the residue was partitioned between EtOAc (25 mL) and H₂O (10 mL). The two phases were separated, and the aqueous phase was extracted with EtOAc (2×25 mL). The combined organic phases were dried over Na₂SO₄ and concentrated in vacuo. The crude residue was purified by flash chromatography (Hexane/EtOAc 1:1) to give 97-F (450 mg, 85% yield). MS(ESI) m/z 407.1 (M+H)⁺. ¹H NMR (600 MHz, Chloroform-d) δ 8.28 (s, 1H), 4.93-4.80 (m, 1H), 4.57 (t, J=7.6 Hz, 2H), 4.18 (t, J=7.6 Hz, 2H), 2.27-2.18 (m, 2H), 2.04-1.96 (m, 2H), 1.93-1.85 (m, 2H), 1.66-1.60 (m, 2H), 1.53 (s, 9H).

### Step 2: Synthesis of 7′-cyclopentyl-2′-((4-(4-methylpiperazin-1-yl)phenyl)amino)spiro[pyrrolidine-3,5′-pyrrolo[2,3-d]pyrimidine]-2,6′(7′H)-dione (Compound C101)

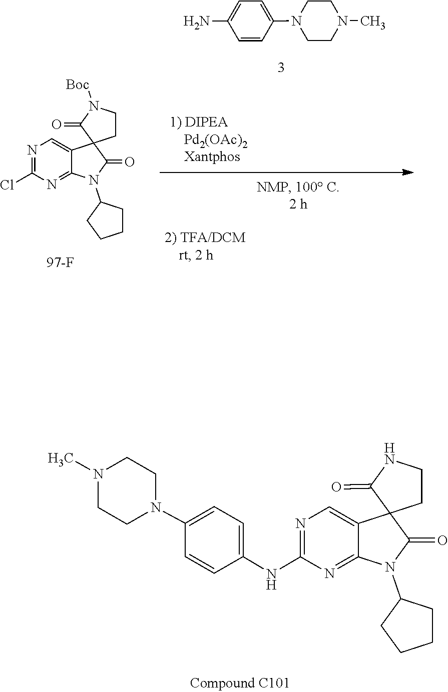 [SMILES: CN1CCN(c2ccc(N)cc2)CC1 ; CC(C)(C)OC(=O)N1CCC2(C1=O)C(=O)N(C1CCCC1)c1nc(Cl)ncc12 ; CN1CCN(c2ccc(Nc3ncc4c(n3)N(C3CCCC3)C(=O)C43CCNC3=O)cc2)CC1]

Compound C101 was synthesized following the general procedure I, 3 mg (2% yield). MS(ESI) m/z 462.3 (M+H)⁺. ¹H NMR (600 MHz, Chloroform-d) δ 8.17 (s, 1H), 7.81 (s, 1H), 7.56 (d, J=8.7 Hz, 2H), 6.91 (d, J=8.9 Hz, 2H), 4.86 (p, J=8.8 Hz, 1H), 4.78 (t, J=8.3 Hz, 2H), 3.96 (t, J=8.3 Hz, 2H), 3.35 (s, 3H), 3.03 (s, 5H), 2.68 (s, 2H), 2.23 (q, J=10.3, 8.6 Hz, 2H), 1.91 (s, 3H), 1.57 (s, 4H).

### Example 98: Compound C102

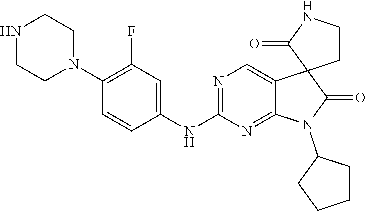 [SMILES: O=C1NCCC12C(=O)N(C1CCCC1)c1nc(Nc3ccc(N4CCNCC4)c(F)c3)ncc12]

### Synthesis of 7′-cyclopentyl-2′-((3-fluoro-4-(piperazin-1-yl)phenyl)amino)spiro[pyrrolidine-3,5′-pyrrolo[2,3-d]pyrimidine]-2,6′(7′H)-dione (Compound C102)

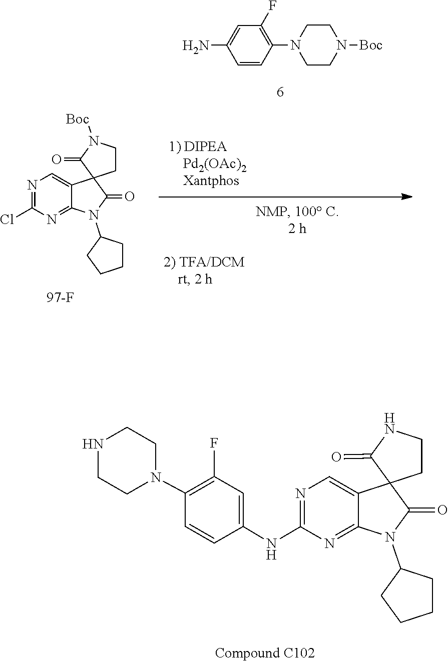 [SMILES: CC(C)(C)OC(=O)N1CCN(c2ccc(N)cc2F)CC1 ; CC(C)(C)OC(=O)N1CCC2(C1=O)C(=O)N(C1CCCC1)c1nc(Cl)ncc12 ; O=C1NCCC12C(=O)N(C1CCCC1)c1nc(Nc3ccc(N4CCNCC4)c(F)c3)ncc12]

Compound C102 was synthesized following the general procedure I, 10 mg (9% yield). MS(ESI) m/z 466.3 (M+H)⁺.

### Example 99: Compound C103

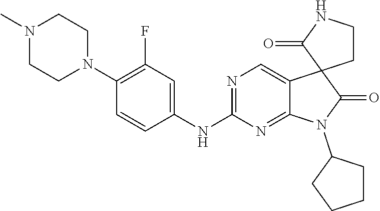 [SMILES: CN1CCN(c2ccc(Nc3ncc4c(n3)N(C3CCCC3)C(=O)C43CCNC3=O)cc2F)CC1]

### Synthesis of 7′-cyclopentyl-2′-((3-fluoro-4-(4-methylpiperazin-1-yl)phenyl)amino)spiro[pyrrolidine-3,5′-pyrrolo[2,3-d]pyrimidine]-2,6′(7′H)-dione (Compound C103)

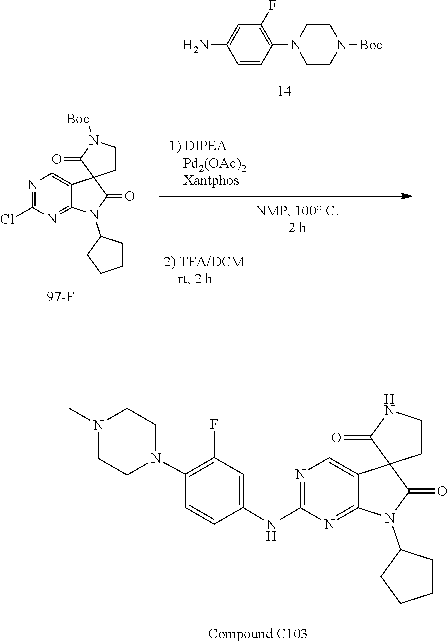 [SMILES: CC(C)(C)OC(=O)N1CCN(c2ccc(N)cc2F)CC1 ; CC(C)(C)OC(=O)N1CCC2(C1=O)C(=O)N(C1CCCC1)c1nc(Cl)ncc12 ; CN1CCN(c2ccc(Nc3ncc4c(n3)N(C3CCCC3)C(=O)C43CCNC3=O)cc2F)CC1]

Compound 99 was synthesized following the general procedure I, 6 mg (6% yield). MS(ESI) m/z 480.3 (M+H)⁺. ¹H NMR (600 MHz, Chloroform-d) δ 8.24 (s, 1H), 8.03 (s, 1H), 7.77-7.68 (m, 1H), 7.11 (d, J=8.7 Hz, 1H), 6.92 (t, J=9.1 Hz, 1H), 4.95-4.82 (m, 1H), 4.75 (t, J=8.2 Hz, 1H), 3.93 (t, J=8.2 Hz, 1H), 3.20 (s, 2H), 2.91 (s, 3H), 2.57 (s, 2H), 2.41-2.26 (m, 2H), 2.05-1.96 (m, 1H), 1.96-1.88 (m, 1H), 1.81-1.40 (m, 10H).

### Example 100: Compound C104

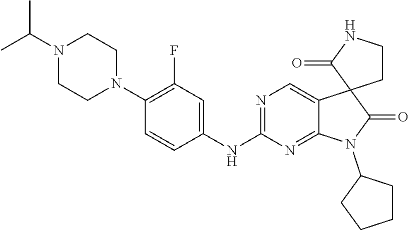 [SMILES: CC(C)N1CCN(c2ccc(Nc3ncc4c(n3)N(C3CCCC3)C(=O)C43CCNC3=O)cc2F)CC1]

### Synthesis of 7′-cyclopentyl-2′-((3-fluoro-4-(4-isopropylpiperazin-1-yl)phenyl)amino)spiro[pyrrolidine-3,5′-pyrrolo[2,3-d]pyrimidine]-2,6′(7′H)-dione (Compound C104)

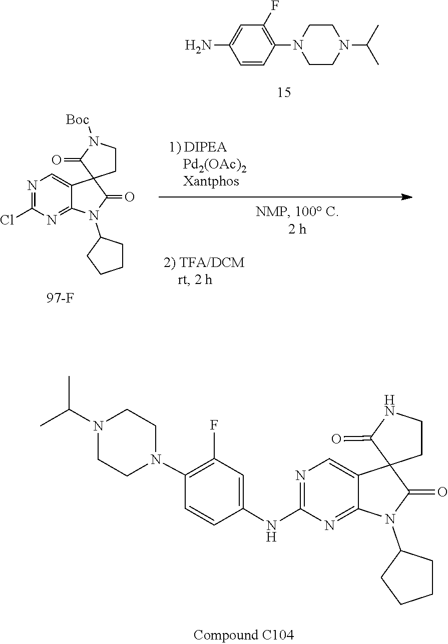 [SMILES: CC(C)N1CCN(c2ccc(N)cc2F)CC1 ; CC(C)(C)OC(=O)N1CCC2(C1=O)C(=O)N(C1CCCC1)c1nc(Cl)ncc12 ; CC(C)N1CCN(c2ccc(Nc3ncc4c(n3)N(C3CCCC3)C(=O)C43CCNC3=O)cc2F)CC1]

Compound C104 was synthesized following the general procedure I, 5 mg (5% yield). MS(ESI) m/z 508.3 (M+H)⁺. ¹H NMR (600 MHz, Chloroform-d) δ 8.25 (s, 1H), 8.08 (s, 1H), 7.74 (dd, J=14.6, 2.5 Hz, 1H), 7.09 (dd, J=9.2, 2.4 Hz, 1H), 6.92 (t, J=9.0 Hz, 1H), 4.89 (p, J=9.0 Hz, 1H), 4.75 (t, J=8.2 Hz, 2H), 3.92 (t, J=8.2 Hz, 2H), 3.27 (s, 4H), 3.06 (s, 4H), 2.39-2.28 (m, 2H), 2.06-1.97 (m, 2H), 1.97-1.87 (m, 2H), 1.72-1.65 (m, 4H), 1.28 (s, 6H).

### Example 101: Compound C105

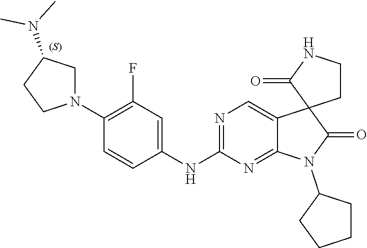

### Synthesis of 7′-cyclopentyl-2′-((4-((S)-3-(dimethylamino)pyrrolidin-1-yl)-3-fluorophenyl)amino)spiro[pyrrolidine-3,5′-pyrrolo[2,3-d]pyrimidine]-2,6′(7′H)-dione (Compound C105)

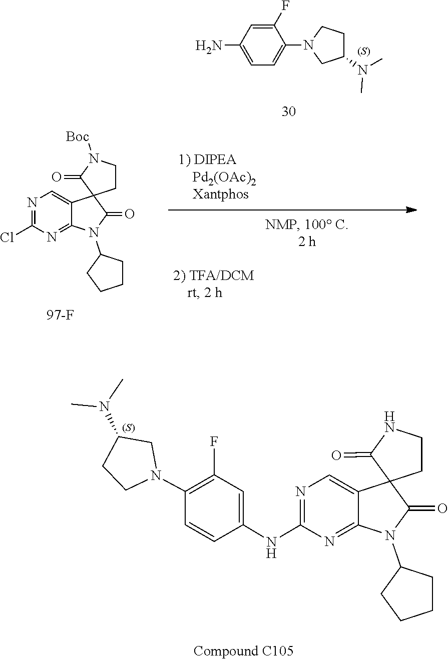

Compound C105 was synthesized following the general procedure I, 5 mg (3% yield). MS(ESI) m/z 494.3 (M+H)⁺.

### Example 102: Compound C106

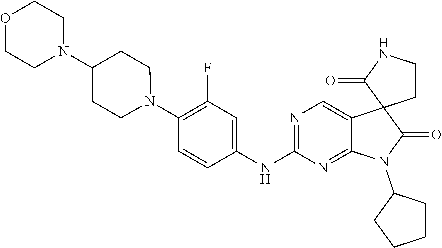 [SMILES: O=C1NCCC12C(=O)N(C1CCCC1)c1nc(Nc3ccc(N4CCC(N5CCOCC5)CC4)c(F)c3)ncc12]

### Synthesis of 7′-cyclopentyl-2′-((3-fluoro-4-(4-morpholinopiperidin-1-yl)phenyl)amino)spiro[pyrrolidine-3,5′-pyrrolo[2,3-d]pyrimidine]-2,6′(7′H)-dione (Compound C106)

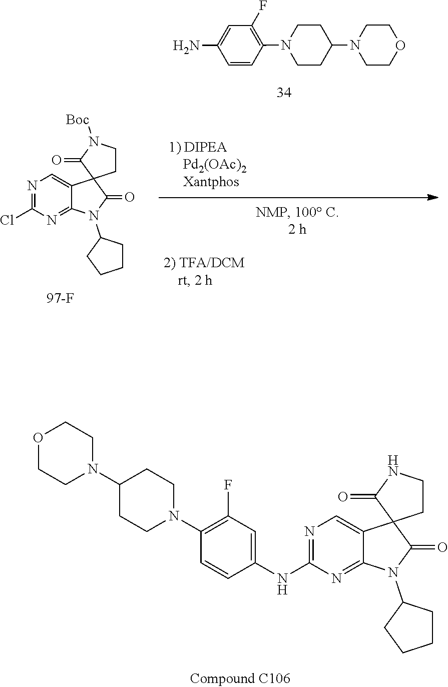 [SMILES: Nc1ccc(N2CCC(N3CCOCC3)CC2)c(F)c1 ; CC(C)(C)OC(=O)N1CCC2(C1=O)C(=O)N(C1CCCC1)c1nc(Cl)ncc12 ; O=C1NCCC12C(=O)N(C1CCCC1)c1nc(Nc3ccc(N4CCC(N5CCOCC5)CC4)c(F)c3)ncc12]

Compound C106 was synthesized following the general procedure I, 8 mg (5% yield). MS(ESI) m/z 550.3 (M+H)⁺. ¹H NMR (600 MHz, Chloroform-d) δ 8.24 (s, 1H), 8.11 (s, 1H), 7.72 (dd, J=14.8, 2.5 Hz, 1H), 7.06-7.01 (m, 1H), 6.90 (t, J=9.1 Hz, 1H), 4.89 (p, J=9.0 Hz, 1H), 4.73 (t, J=8.1 Hz, 2H), 3.90 (t, J=8.1 Hz, 2H), 3.75 (t, J=4.7 Hz, 4H), 3.45 (d, J=11.6 Hz, 2H), 2.70-2.64 (m, 2H), 2.61 (t, J=4.7 Hz, 3H), 2.41-2.30 (m, 3H), 2.07-1.97 (m, 2H), 1.97-1.86 (m, 4H), 1.81-1.72 (m, 2H), 1.71-1.64 (m, 3H).

### Example 103: Compound C99

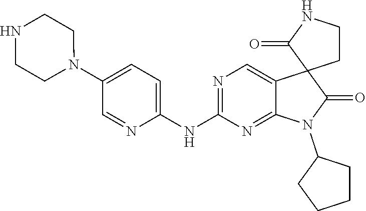 [SMILES: O=C1NCCC12C(=O)N(C1CCCC1)c1nc(Nc3ccc(N4CCNCC4)cn3)ncc12]

### Step 1: Synthesis of tert-butyl 7′-cyclopentyl-2′-(methylsulfinyl)-2,6′-dioxo-6′,7′-dihydrospiro[pyrrolidine-3,5′-pyrrolo[2,3-d]pyrimidine]-1-carboxylate (103-M)

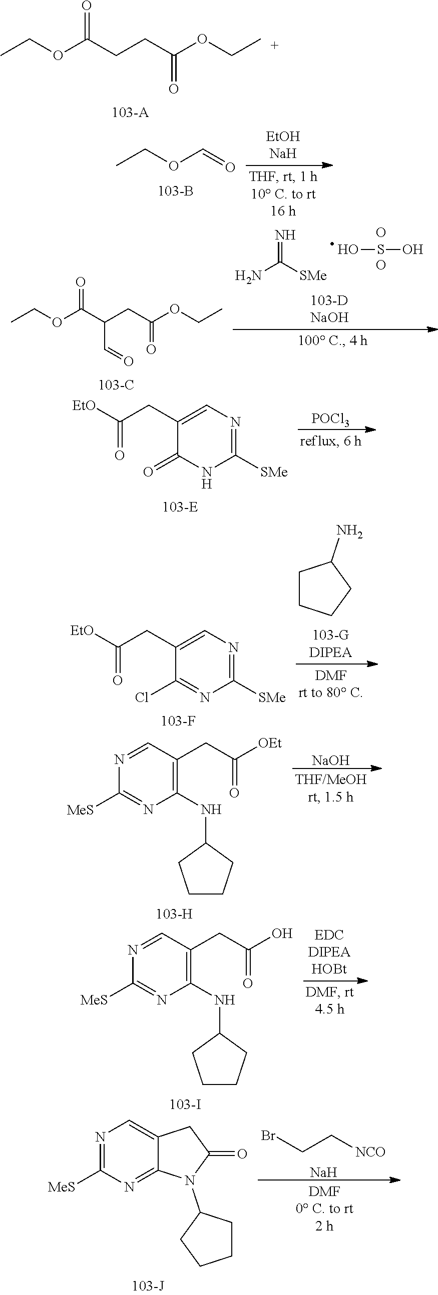

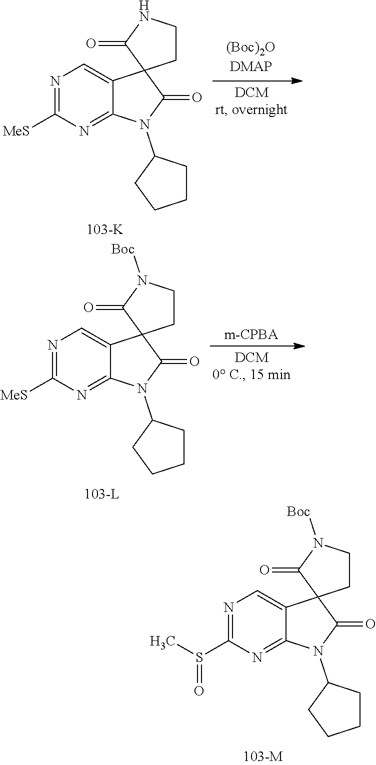

Synthesis of 103-C: (Ref: WO 2009152027): Ethanol (41 g, 0.861 mol) was added dropwise to a suspension of sodium hydride (60 wt %, 34.5 g, 0.861 mol) in tetrahydrofuran (400 mL); the mixture was stirred at room temperature for 1 hour. Upon cooling to 10° C. (cold water bath), solutions of diethyl succinate 103-A (150 g, 0.861 mol) in tetrahydrofuran (150 mL) followed by ethyl formate 103-B (64 g, 0.861 mol) in tetrahydrofuran (150 mL) were added dropwise. After stirring for 16 h at room temperature, water (500 mL) was added and the resulting solution was washed with diethyl ether (2×250 mL). The water phase was acidified with 50% aq. H₂SO₄ and the resulting suspension was extracted with diethyl ether (2×250 mL). The combined organic layer was washed with water (3×200 mL), dried with sodium sulfate, filtrated through Celite, concentrated under reduced pressure and used as such for next step 103-C (120 g, 69% yield. MS(ESI) m/z 203.2 (M+H)⁺.

Synthesis of 103-E: (Ref: WO 2009152027): To a solution of diethyl 2-formylsuccinate 103-C (120 g, 0.59 mol) and S-methylthiourea semi-sulfate 103-D (82.1 g, 0.59 mol) in 500 mL of water, a solution of sodium hydroxide (35.4 g, 0.885 mol) in water (100 mL) was added, The reaction mixture was stirred at 100° C. for 1.5 hours, cooled to room temperature, and acidified with slow addition of acetic acid. After stirring at room temperature for 15 min, the resulting precipitate was filtered, thoroughly washed with water, and finally dried by evaporation with acetonitrile (3 times) to afford ethyl [2-(methylthio)-4-oxo-1,4-dihydropyrimidin-5-yl]acetate 103-E (63 g, 47% yield). MS(ESI) m/z 229.1 (M+H)⁺.

Synthesis of 103-F: (Ref: WO 2009152027): A mixture of ethyl [2-(methylthio)-4-oxo-1,4-dihydropyrimidin-5-yl]acetate 103-E (63 g, 0.26 mol) in POCl₃ (350 mL) was refluxed for 6 hours and then concentrated under reduced pressure. The residue was twice re-evaporated with benzene and purified by column chromatography on silica gel (ethyl acetate-hexanes, 1:3) to afford ethyl [4-chloro-2-(methylthio)pyrimidin-5-yl]acetate 103-F (62 g, 97% yield). MS(ESI) m/z 247.1 (M+H)⁺.

Synthesis of 103-H: A mixture of 103-F (17.5 g, 71 mmol), 103-G (15.1 g, 177.5 mmol) and N, N-diisopropylethyl amine (50 mL, 284 mmol) in anhydrous DMF (100 mL) was stirred at RT for overnight and then at 80° C. for 2 h. The reaction mixture was then diluted with ethyl acetate and washed with 2× water and 4× brine, dried over sodium sulfate, filtered and concentrated. The obtained crude 103-H residue is used as such for next step. MS(ESI) m/z 296.2 (M+H)⁺.

Synthesis of 103-1: To 103-H in THF/MeOH (140 mL, 70 mL/70 mL), was added 2N NaOH (70 mL) at RT and stirred for 1 h. The reaction mixture is concentrated, taken in 100 mL of water and washed 2×EtOAc. The aqueous layer is acidified to pH-2 with 2 N HCl and the solid is collected by filtration, washed with water and dried to give 103-I (11.5 g, 61% yield, over two steps). MS(ESI) m/z 268.1 (M+H)⁺. ¹H NMR (600 MHz, Chloroform-d) δ 7.76 (s, 1H), 4.53-4.38 (m, 1H), 3.35 (s, 2H), 2.53 (s, 3H), 2.13-1.96 (m, 2H), 1.86-1.70 (m, 2H), 1.67-1.50 (m, 4H).

Synthesis of 103-J: 103-I (11.5 g, 43 mmol), EDC (12.3 g, 64.5 mmol), HOBt (9.9 g, 64.5 mmol), and diisopropylethylamine (11.2 mL, 64.5 mmol) are dissolved in dimethylformamide (120 mL) and the mixture was stirred for 2 h to achieve complete conversion. The reaction mixture was diluted with ethyl acetate and washed with saturated aqueous sodium hydrogen carbonate, water, then 4× brine. The organic phase is dried (Na₂SO₄), filtered and concentrated. Purification by chromatography on silica gel (hexane:ethyl acetate, 3:2) provided the desired product 103-J (9 g, 84% yield). MS(ESI) m/z 250.1 (M+H)⁺. ¹H NMR (600 MHz, Chloroform-d) δ 8.10 (s, 1H), 4.78 (p, J=8.8 Hz, 1H), 3.52-3.42 (m, 2H), 2.55 (s, 3H), 2.28-2.13 (m, 2H), 2.02-1.93 (m, 2H), 1.93-1.83 (m, 2H), 1.69-1.60 (m, 2H).

Synthesis of 103-K: (Ref: Crosignani et al., J. Med. Chem. 51(7): 2227-2243(2008)): NaH (1.5 g, 24 mmol) was added to a solution of 103-J (4 g, 16 mmol) in DMF (20 mL) at 0° C. The resulting mixture was stirred at RT for 30 min. At this time 2-bromoethyl isocyanate (1.8 mL, 17.6 mmol) was added at RT and the reaction was stirred for 2 h. The reaction mixture was then diluted with ethyl acetate and washed with water and brine, concentrated and used as such for next step (103-K). MS(ESI) m/z 319.1 (M+H)⁺. ¹H NMR (600 MHz, Chloroform-d) δ 8.41 (s, 1H), 8.23 (s, 1H), 4.92 (p, J=8.7 Hz, 1H), 4.76 (t, J=8.3 Hz, 2H), 3.92 (t, J=8.3 Hz, 2H), 2.58 (s, 3H), 2.40-2.23 (m, 2H), 2.06-1.95 (m, 2H), 1.97-1.88 (m, 2H), 1.73-1.63 (m, 2H).

Synthesis of 103-L: (Ref: Putey et al., J. Med. Chem. 52(19): 5916-5925 (2009)): A solution of 103-K (5.1 g, 16 mmol), Boc₂O (7 g, 32 mmol), and a catalytic amount of DMAP (586 mg, 4.8 mmol) in DCM (100 mL) was stirred overnight at room temperature. The reaction mixture was washed with H₂O, brine, dried over Na₂SO₄ and concentrated in vacuo. The crude residue was purified by flash chromatography (Hexane/EtOAc 4:1) to give 103-L (3.8 g, 57% yield, over two steps). MS(ESI) m/z 419.2 (M+H)⁺. ¹H NMR (600 MHz, Chloroform-d) δ 8.31 (s, 1H), 4.98-4.82 (m, 1H), 4.51 (t, J=7.5 Hz, 2H), 4.15 (t, J=7.5 Hz, 2H), 2.57 (s, 3H), 2.30-2.22 (m, 2H), 2.02-1.94 (m, 2H), 1.92-1.83 (m, 2H), 1.70-1.60 (m, 2H), 1.52 (s, 9H).

Synthesis of 103-M: To a solution of 103-L (2 g, 4.8 mmol) in 400 mL DCM was added m-CPBA (1.2 g, 5.3 mmol) in one portion at 0° C. After stirring for 15 min, reaction mixture was washed with saturated NaHCO₃ and brine. The organic layer was dried over sodium sulfate, concentrated, dried, and used as such for next step 103-M (2.1 g, 95% yield). MS(ESI) m/z 435.2 (M+H)⁺. ¹H NMR (600 MHz, Chloroform-d) δ 8.55 (s, 1H), 7.27 (s, 1H), 5.01-4.89 (m, 1H), 4.70-4.57 (m, 2H), 4.28-4.15 (m, 2H), 2.93 (s, 3H), 2.27-2.18 (m, 2H), 1.99 (s, 2H), 1.95-1.87 (m, 2H), 1.66 (s, 2H), 1.54 (s, 9H).

### Step 2: Synthesis of 7′-cyclopentyl-2′-((5-(piperazin-1-yl)pyridin-2-yl)amino)spiro[pyrrolidine-3,5′-pyrrolo[2,3-d]pyrimidine]-2,6′(7′H)-dione (Compound C99)

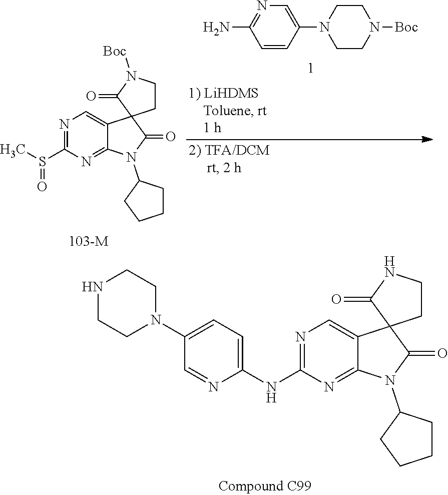 [SMILES: CC(C)(C)OC(=O)N1CCN(c2ccc(N)nc2)CC1 ; CS(=O)c1ncc2c(n1)N(C1CCCC1)C(=O)C21CCN(C(=O)OC(C)(C)C)C1=O ; O=C1NCCC12C(=O)N(C1CCCC1)c1nc(Nc3ccc(N4CCNCC4)cn3)ncc12]

Compound C99 was synthesized following the general procedure D, 9 mg (10% yield). MS(ESI) m/z 449.3 (M+H)⁺.

### Example 104: Compound C100

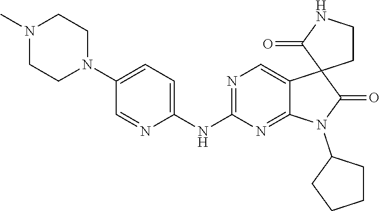 [SMILES: CN1CCN(c2ccc(Nc3ncc4c(n3)N(C3CCCC3)C(=O)C43CCNC3=O)nc2)CC1]

### Synthesis of 7′-cyclopentyl-2′-((5-(4-methylpiperazin-1-yl)pyridin-2-yl)amino)spiro[pyrrolidine-3,5′-pyrrolo[2,3-d]pyrimidine]-2,6′(7′H)-dione (Compound C100)

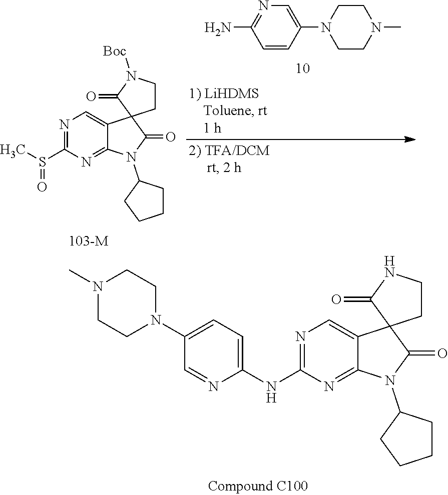 [SMILES: CN1CCN(c2ccc(N)nc2)CC1 ; CS(=O)c1ncc2c(n1)N(C1CCCC1)C(=O)C21CCN(C(=O)OC(C)(C)C)C1=O ; CN1CCN(c2ccc(Nc3ncc4c(n3)N(C3CCCC3)C(=O)C43CCNC3=O)nc2)CC1]

Compound C100 was synthesized following the general procedure D, 10 mg (11% yield). MS(ESI) m/z 463.3 (M+H)⁺. ¹H NMR (600 MHz, Chloroform-d) δ 8.26 (s, 1H), 8.21 (s, 1H), 8.08 (s, 1H), 7.98 (s, 1H), 7.33 (dd, J=9.1, 3.0 Hz, 1H), 4.90 (p, J=8.9 Hz, 1H), 4.76 (t, J=8.2 Hz, 2H), 3.93 (t, J=8.2 Hz, 2H), 3.36 (s, 4H), 3.06 (s, 4H), 2.73 (s, 3H), 2.38-2.25 (m, 2H), 2.00 (d, J=7.4 Hz, 2H), 1.93 (d, J=8.4 Hz, 2H), 1.69 (d, J=5.7 Hz, 2H).

### Example 105: Compound C14

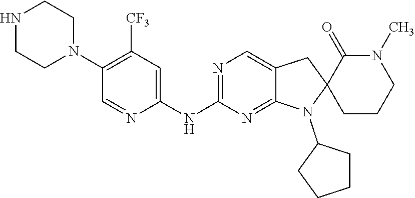 [SMILES: CN1CCCC2(Cc3cnc(Nc4cc(C(F)(F)F)c(N5CCNCC5)cn4)nc3N2C2CCCC2)C1=O]

### Synthesis of 7′-cyclopentyl-1-methyl-2′-((5-(piperazin-1-yl)-4-(trifluoromethyl)pyridin-2-yl)amino)-5′,7′-dihydrospiro[piperidine-3,6′-pyrrolo[2,3-d]pyrimidin]-2-one (Compound C14)

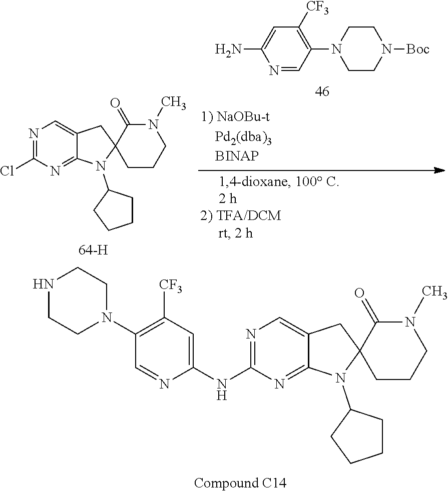 [SMILES: CC(C)(C)OC(=O)N1CCN(c2cnc(N)cc2C(F)(F)F)CC1 ; CN1CCCC2(Cc3cnc(Cl)nc3N2C2CCCC2)C1=O ; CN1CCCC2(Cc3cnc(Nc4cc(C(F)(F)F)c(N5CCNCC5)cn4)nc3N2C2CCCC2)C1=O]

Compound C14 was synthesized following the general procedure A, 45 mg (34% yield). MS(ESI) m/z 531.3 (M+H)⁺.

### Separation of two enantiomers of tert-butyl 7′-cyclopentyl-2′-(methylthio)-2-oxo-5′,7′-dihydrospiro[pyrrolidine-3,6′-pyrrolo[2,3-d]pyrimidine]-1-carboxylate (26-L)

### Analytical Chiral Column Chromatography

### Column: Chiralcel OZ-H 4.6×250 mm 5 μm; Mobile Phase: Acetonitrile

Flow Rate: 1.0 mL/min; wavelengths: 254 nm and 280 nm

### Preparative Chiral Chromatography Conditions

Column: Chiralcel OZ-H 21×250 mm, 5 micron; Mobile phase: Acetonitrile; Flow rate: 4 mL/min; Wavelength: 254 nm

### Retention Times (t_{R}): 4.5 min. (Enantiomer-1, E1), 6.9 min. (Enantiomer-2, E2)

Standard chromatographic techniques were used to isolate the required two enantiomers, E1 and E2 as off-white solids (concentrated and lyophilized).
About 8 g of racemic 26-L was used to isolate the following amounts:
Enantiomer-1 (E1): Yield: 3.65 g (46%), Purity: >98%; Optical purity: % ee >96%
Enantiomer-2 (E2): Yield: 4.35 g (44%); Purity: >98%; Optical purity: % ee >96%

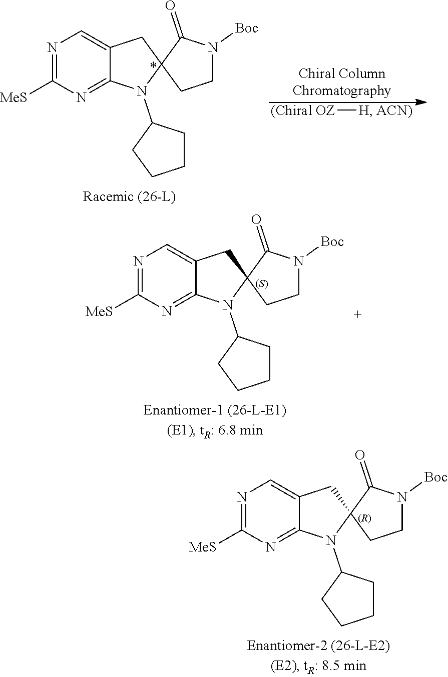

### Synthesis of tert-butyl 7′-cyclopentyl-2′-(methylsulfinyl)-2-oxo-5′,7′-dihydrospiro[pyrrolidine-3,6′-pyrrolo[2,3-d]pyrimidine]-1-carboxylate (121-I-E1sulfoxide)

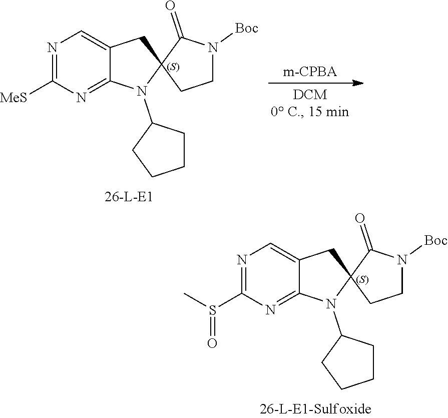

E1-sulfoxide (diastereomers) was synthesized according to the procedure (using 1.1 equivalent of m-CPBA) described for making racemic sulfoxide 26-M. Yield: 850 mg (97%)

MS(ESI) m/z 421.2 (M+H)⁺. ¹H NMR (600 MHz, Chloroform-d) δ 7.98 (d, J=10.1 Hz, 1H), 3.99-3.87 (m, 1H), 3.67-3.49 (m, 2H), 3.30 (d, J=17.3, 1H), 3.05 (dd, J=16.8, 3.0 Hz, 2.86 (s, 3H), 2.44-2.23 (m, 2H), 2.21-2.10 (m, 2H), 2.00-1.85 (m, 3H), 1.81-1.69 (m, 1H), 1.66 (s, 2H), 1.56 (s, 9H).

### Synthesis of tert-butyl 7′-cyclopentyl-2′-(methylsulfinyl)-2-oxo-5′,7′-dihydrospiro[pyrrolidine-3,6′-pyrrolo[2,3-d]pyrimidine]-1-carboxylate (121-I-E2sulfoxide)

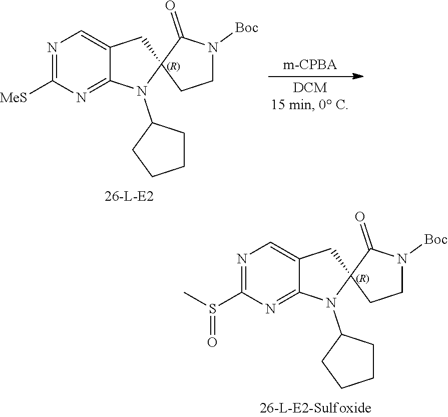

E2-sulfoxide (diastereomers) was synthesized according to the procedure (using 1.1 equivalent of m-CPBA) described for making racemic sulfoxide 26-M. Yield: 850 mg (97%). MS(ESI) m/z 421.2 (M+H)⁺.

### Example-106 (VP-NR10-130-E1, SPV-454)

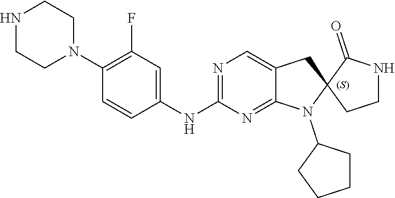

### Synthesis of (S)-7′-cyclopentyl-2′-((3-fluoro-4-(piperazin-1-yl)phenyl)amino)-5′,7′-dihydrospiro[pyrrolidine-3,6′-pyrrolo[2,3-d]pyrimidin]-2-one

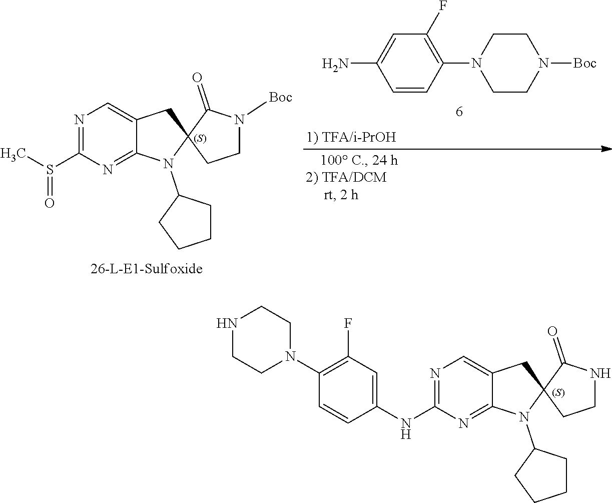

was synthesized following the general procedure C, 15 mg (33% yield). MS(ESI) m/z 452.2 (M+H)⁺. ¹H NMR (600 MHz, DMSO-d₆) δ 8.82 (s, 1H), 7.94 (s, 1H), 7.70 (dd, J=15.8, 2.4 Hz, 1H), 7.53 (d, J=1.4 Hz, 1H), 7.17 (dd, J=8.7, 2.4 Hz, 1H), 6.81 (dd, J=10.0, 8.8 Hz, 1H), 3.40 (d, J=8.5 Hz, 3H), 3.22-3.13 (m, 7H), 2.92-2.82 (m, 3H), 2.31-2.16 (m, 4H), 2.08 (ddd, J=12.9, 6.7, 2.0 Hz, 1H), 1.79-1.75 (m, 2H), 1.67-1.61 (m, 2H), 1.50-1.39 (m, 2H).

### Example-107 (VP-NR10-131-E1, SPV-455)

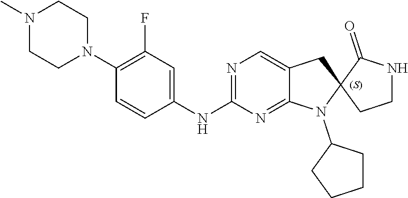

### Synthesis of (S)-7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one

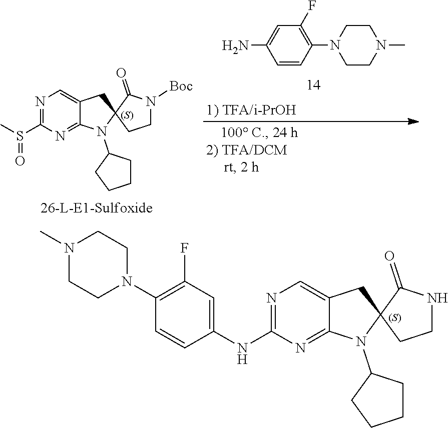

was synthesized following the general procedure C, 16 mg (34% yield). MS(ESI) m/z 466.2 (M+H)⁺. ¹H NMR (600 MHz, DMSO-d₆) δ 9.18 (s, 1H), 8.62 (s, 2H), 8.03 (s, 1H), 7.90 (s, 1H), 7.50 (d, J=8.9 Hz, 2H), 6.87 (d, J=9.0 Hz, 2H), 5.27 (p, J=8.7 Hz, 1H), 3.22-3.11 (m, 10H), 2.96 (d, J=15.8 Hz, 1H), 2.67 (d, J=15.8 Hz, 1H), 2.32 (ddd, J=12.8, 7.8, 5.0 Hz, 1H), 2.02-1.92 (m, 2H), 1.88-1.75 (m, 3H), 1.75-1.66 (m, 2H), 1.51-1.41 (m, 2H).

### Example-108 (VP-NR10-132-E2, SPV-456)

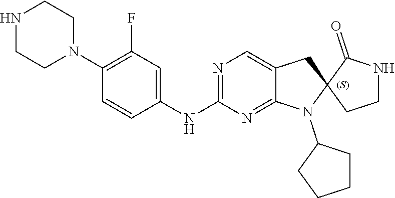

### Synthesis of (S)-7′-cyclopentyl-2′-((3-fluoro-4-(piperazin-1-yl)phenyl)amino)-5′,7′-dihydrospiro[pyrrolidine-3,6′-pyrrolo[2,3-d]pyrimidin]-2-one

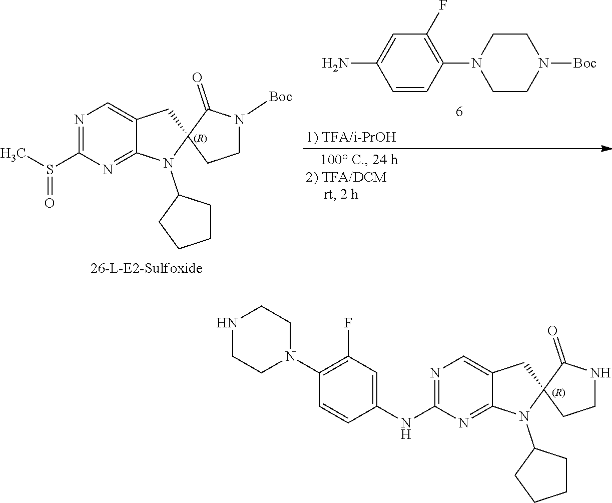

was synthesized following the general procedure C, 12 mg (27% yield). MS(ESI) m/z 452.2 (M+H)⁺. ¹H NMR (600 MHz, DMSO-d₆) δ 9.18 (s, 1H), 8.62 (s, 2H), 8.03 (s, 1H), 7.90 (s, 1H), 7.50 (d, J=8.9 Hz, 2H), 6.87 (d, J=9.0 Hz, 2H), 5.27 (p, J=8.7 Hz, 1H), 3.22-3.11 (m, 10H), 2.96 (d, J=15.8 Hz, 1H), 2.67 (d, J=15.8 Hz, 1H), 2.32 (ddd, J=12.8, 7.8, 5.0 Hz, 1H), 2.02-1.92 (m, 2H), 1.88-1.75 (m, 3H), 1.75-1.66 (m, 2H), 1.51-1.41 (m, 2H).

### Example-109 (VP-NR10-133-E2, SPV-457)

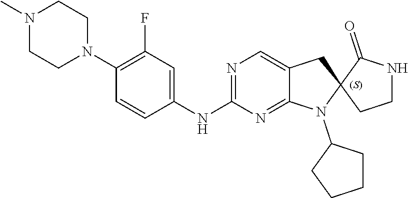

### Synthesis of (S)-7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one

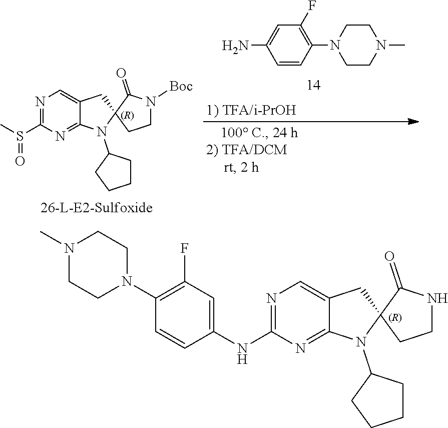

was synthesized following the general procedure C, 16 mg (34% yield). MS(ESI) m/z 466.2 (M+H)⁺. ¹H NMR (600 MHz, DMSO-d₆) δ 8.14 (s, 1H), 7.54 (s, 1H), 7.50 (d, J=14.2 Hz, 1H), 7.09 (dd, J=8.7, 2.4 Hz, 1H), 7.03 (dd, J=19.9, 10.9 Hz, 1H), 3.50 (t, J=8.9 Hz, 2H), 3.25-3.13 (m, 6H), 3.01 (d, J=16.8 Hz, 1H), 2.93 (d, J=16.6, 1.8 Hz, 1H), 2.80 (s, 3H), 2.44 (s, 2H), 2.38-2.30 (m, 1H), 2.20-2.12 (m, 2H), 2.10-1.99 (m, 2H), 1.73-1.67 (m, 1H), 1.67-1.57 (m, 3H), 1.45-1.35 (m, 2H).

### Example-110 (VP-7-302A-E1, SPV-487)

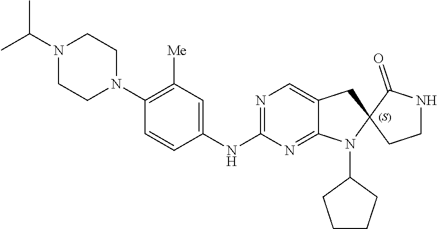

### (S)-7′-cyclopentyl-2′-((4-(4-isopropylpiperazin-1-yl)-3-methylphenyl)amino)-5′,7′-dihydrospiro[pyrrolidine-3,6′-pyrrolo[2,3-d]pyrimidin]-2-one

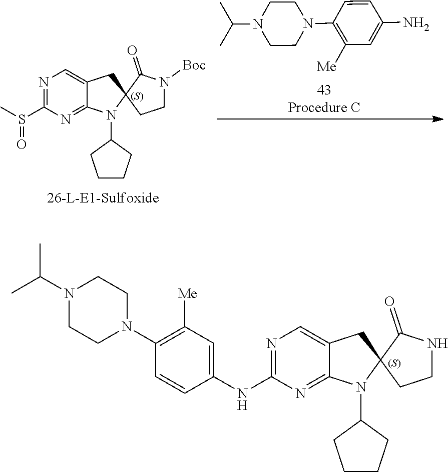

was synthesized following the general procedure C, 20 mg (41% yield). MS(ESI) m/z 490.3 (M+H)⁺. 1H NMR (600 MHz, DMSO-d₆) δ 8.22 (s, 1H), 7.57 (s, 1H), 7.30 (d, J=2.6 Hz, 1H), 7.25 (dd, J=8.6, 2.6 Hz, 1H), 7.07 (d, J=8.5 Hz, 1H), 3.59-3.53 (m, 2H), 3.49 (d, J=11.5 Hz, 2H), 3.30-3.24 (m, 3H), 3.24-3.16 (m, 4H), 3.10-3.04 (m, 1H), 3.03-2.96 (m, 3H), 2.46-2.37 (m, 2H), 2.28 (s, 3H), 2.24 (ddd, J=13.2, 6.8, 2.2 Hz, 1H), 2.20-2.11 (m, 1H), 2.10-2.00 (m, 1H), 1.78-1.72 (m, 1H), 1.72-1.66 (m, 1H), 1.46-1.37 (m, 2H), 1.31 (d, J=6.5 Hz, 6H).

### Example-111 (VP-7-296B-E1, SPV-490)

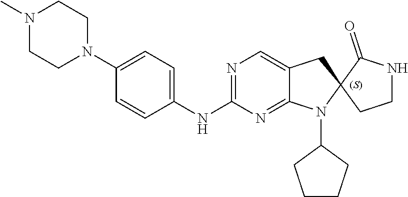

### (S)-7′-cyclopentyl-2′-((4-(4-methylpiperazin-1-yl)phenyl)amino)-5′,7′-dihydrospiro[pyrrolidine-3,6′-pyrrolo[2,3-d]pyrimidin]-2-one

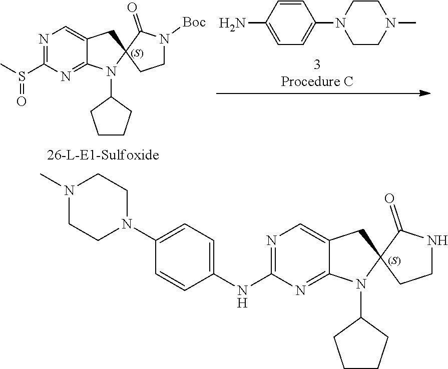

was synthesized following the general procedure C, 12 mg (27% yield). MS(ESI) m/z 448.3 (M+H)⁺. ¹H NMR (600 MHz, DMSO-d₆) δ 8.22 (s, 1H), 7.52 (s, 1H), 7.33 (s, 2H), 7.03 (d, J=8.5 Hz, 2H), 3.81 (s, 2H), 3.55 (dd, J=29.3, 7.7 Hz, 3H), 3.06 (d, J=16.9 Hz, 1H), 3.02-2.96 (m, 1H), 2.91 (s, 2H), 2.87 (s, 3H), 2.45-2.37 (m, 2H), 2.24 (dd, J=13.6, 6.6 Hz, 1H), 2.14 (s, 2H), 2.05 (s, 1H), 1.75 (d, J=8.7 Hz, 2H), 1.71-1.64 (m, 2H), 1.60 (s, 2H), 1.47-1.37 (m, 2H).

### Example-112 (VP-7-299A-E1, SPV-491)

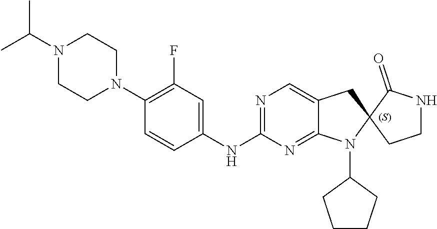

### Synthesis of (S)-7′-cyclopentyl-2′-((3-fluoro-4-(4-isopropylpiperazin-1-yl)phenyl)amino)-5′,7′-dihydrospiro[pyrrolidine-3,6′-pyrrolo[2,3-d]pyrimidin]-2-one

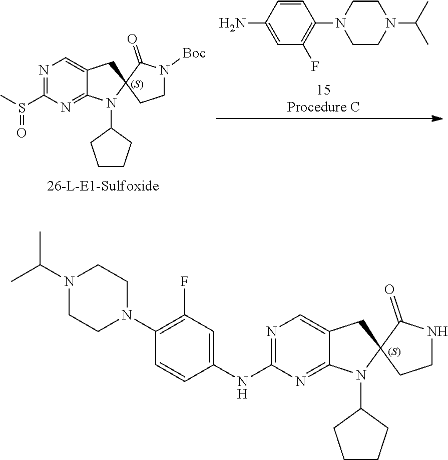

was synthesized following the general procedure C, 20 mg (41% yield). MS(ESI) m/z 494.3 (M+H)⁺. ¹H NMR (600 MHz, DMSO-d₆) δ 8.24 (s, 1H), 7.61 (d, J=2.0 Hz, 1H), 7.53 (d, J=14.2 Hz, 1H), 7.18-7.10 (m, 2H), 3.62-3.54 (m, 3H), 3.55-3.48 (m, 5H), 3.24-3.19 (m, 2H), 3.12-2.99 (m, 5H), 2.46-2.37 (m, 2H), 2.25 (ddd, J=13.2, 6.8, 2.2 Hz, 1H), 2.22-2.15 (m, 1H), 2.13-2.07 (m, 1H), 1.81-1.75 (m, 1H), 1.74-1.67 (m, 1H), 1.50-1.41 (m, 2H), 1.30 (d, J=6.6 Hz, 6H).

### Example-113 (VP-7-304A-E1, SPV-493)

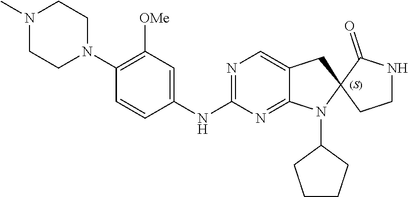

### Synthesis of (S)-7′-cyclopentyl-2′-((3-methoxy-4-(4-methylpiperazin-1-yl)phenyl)amino)-5′,7′-dihydrospiro[pyrrolidine-3,6′-pyrrolo[2,3-d]pyrimidin]-2-one

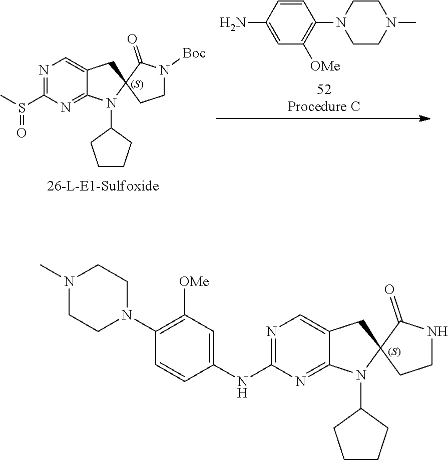

was synthesized following the general procedure C, 20 mg (42% yield). MS(ESI) m/z 478.3 (M+H)⁺. ¹H NMR (600 MHz, DMSO-d₆) δ 8.10 (s, 1H), 7.48 (s, 1H), 7.04 (s, 2H), 6.85 (d, J=8.3 Hz, 1H), 3.71 (s, 3H), 3.53-3.47 (m, 1H), 3.47-3.34 (m, 3H), 3.24-3.17 (m, 5H), 2.97 (d, J=16.4 Hz, 1H), 2.90 (d, J=16.4 Hz, 2H), 2.79 (s, 3H), 2.36-2.28 (m, 2H), 2.14 (ddd, J=13.2, 6.8, 2.1 Hz, 1H), 2.09-2.06 (m, 1H), 2.01-1.98 (m, 1H), 1.71-1.58 (m, 3H), 1.53 (s, 2H), 1.39-1.29 (m, 2H).

### Example-114 (VP-7-311A-E1, SPV-494)

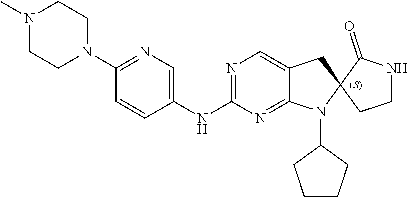

### (S)-7′-cyclopentyl-2′-((6-(4-methylpiperazin-1-yl)pyridin-3-yl)amino)-5′,7′-dihydrospiro[pyrrolidine-3,6′-pyrrolo[2,3-d]pyrimidin]-2-one

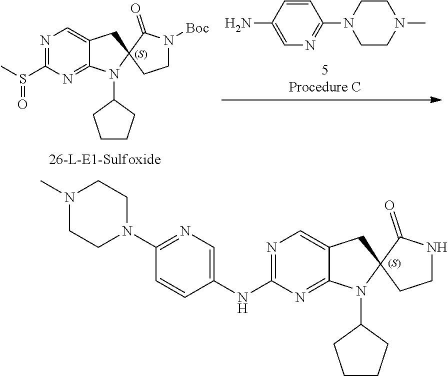

was synthesized following the general procedure C, 21 mg (47% yield). MS(ESI) m/z 449.3 (M+H)⁺. ¹H NMR (600 MHz, DMSO-d₆) δ 8.22 (d, J=3.0 Hz, 2H), 7.73-7.68 (m, 1H), 7.54 (s, 1H), 7.01 (d, J=9.0 Hz, 1H), 3.59-3.53 (m, 2H), 3.32-3.25 (m, 5H), 3.16 (s, 2H), 3.06 (d, J=16.8, 1H), 2.99 (d, J=16.7, 1H), 2.85 (s, 3H), 2.45-2.39 (m, 1H), 2.27-2.20 (m, 1H), 2.12-2.09 (m, 1H), 2.02-1.98 (m, 1H), 1.78-1.70 (m, 2H), 1.70-1.63 (m, 2H), 1.55 (s, 1H), 1.40 (d, J=22.3 Hz, 3H).

### Example-115 (VP-8-3A-E1, SPV-495)

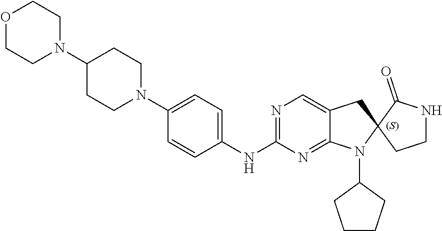

### (S)-7′-cyclopentyl-2′-((4-(4-morpholinopiperidin-1-yl)phenyl)amino)-5′,7′-dihydrospiro[pyrrolidine-3,6′-pyrrolo[2,3-d]pyrimidin]-2-one

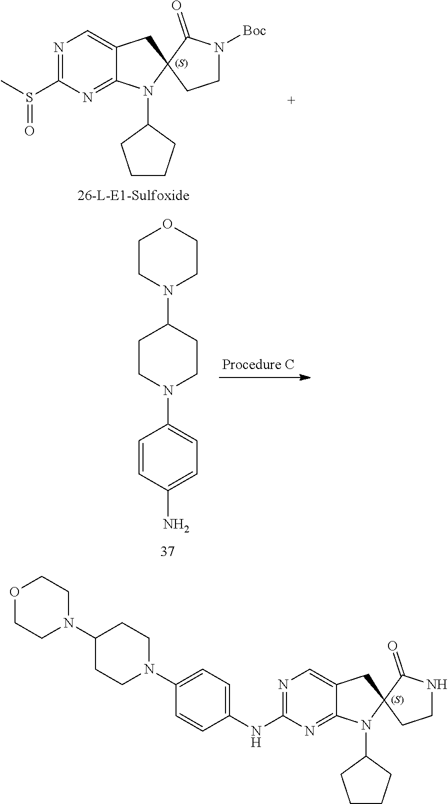

was synthesized following the general procedure C, 34 mg (66% yield). MS(ESI) m/z 518.3 (M+H)⁺. ¹H NMR (600 MHz, DMSO-d₆) δ 8.84 (s, 1H), 8.04 (s, 1H), 7.54 (s, 1H), 7.47 (d, J=8.4 Hz, 2H), 6.88-6.83 (m, 2H), 3.71-3.55 (m, 6H), 3.51-3.45 (m, 1H), 3.29-3.21 (m, 3H), 2.97 (d, J=16.0 Hz, 1H), 2.91 (d, J=15.4 Hz, 1H), 2.81-2.63 (m, 3H), 2.61-2.56 (m, 2H), 2.50 (s, 2H), 2.36-2.27 (m, 2H), 2.21-2.12 (m, 2H), 1.91 (d, J=12.2 Hz, 2H), 1.77 (s, 1H), 1.73-1.66 (m, 2H), 1.57-1.43 (m, 4H).

### Example-116 (VP-8-4B-E1, SPV-496)

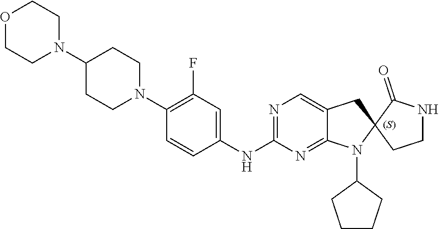

### Synthesis of (S)-7′-cyclopentyl-2′-((3-fluoro-4-(4-morpholinopiperidin-1-yl)phenyl)amino)-5′,7′-dihydrospiro[pyrrolidine-3,6′-pyrrolo[2,3-d]pyrimidin]-2-one

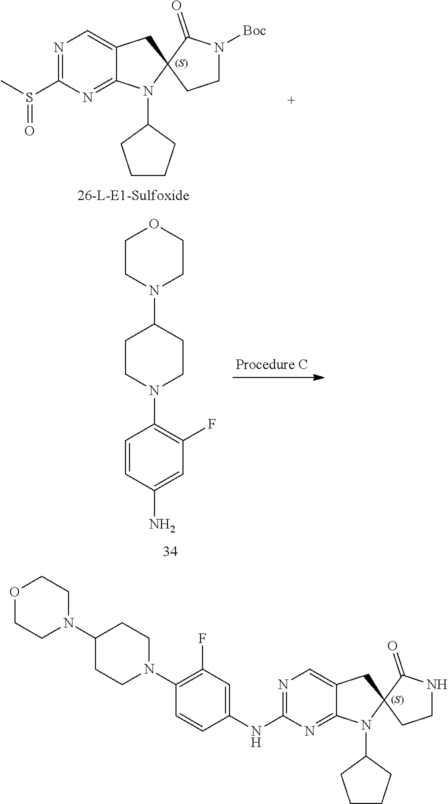

was synthesized following the general procedure C, 10 mg (19% yield). MS(ESI) m/z 536.3 (M+H)⁺. ¹H NMR (600 MHz, DMSO-d₆) δ 7.97 (s, 1H), 7.67 (d, J=15.4 Hz, 1H), 7.53 (s, 1H), 7.18-7.13 (m, 1H), 6.88 (t, J=9.3 Hz, 1H), 3.61 (s, 4H), 3.42 (dd, J=17.9, 8.9 Hz, 1H), 3.22-3.15 (m, 3H), 2.92 (d, J=16.0 Hz, 1H), 2.86 (dd, J=15.8, 1.6 Hz, 1H), 2.61-2.50 (m, 3H), 2.43 (s, 4H), 2.33-2.21 (m, 3H), 2.16 (s, 1H), 2.12-2.06 (m, 1H), 1.99 (s, 3H), 1.74 (s, 1H), 1.70-1.55 (m, 4H), 1.49-1.39 (m, 2H).

### Example-117 (VP-8-5B-E1, SPV-497)

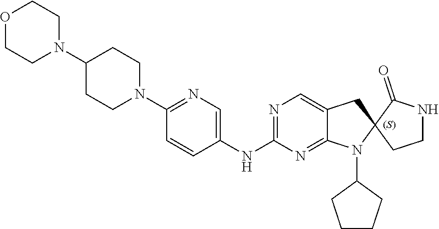

### Synthesis of (S)-7′-cyclopentyl-2′-((6-(4-morpholinopiperidin-1-yl)pyridin-3-yl)amino)-5′,7′-dihydrospiro[pyrrolidine-3,6′-pyrrolo[2,3-d]pyrimidin]-2-one

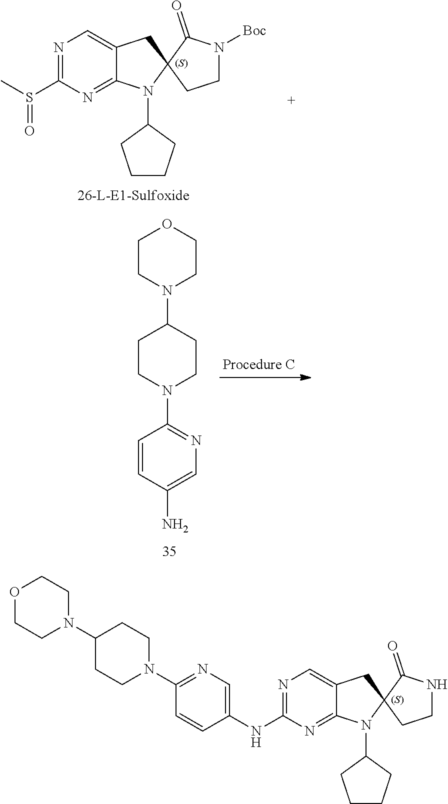

was synthesized following the general procedure C, 13 mg (25% yield). MS(ESI) m/z 519.3 (M+H)⁺. ¹H NMR (600 MHz, DMSO-d₆) δ 8.30 (s, 1H), 8.03 (s, 1H), 7.79 (d, J=7.8 Hz, 1H), 7.54 (s, 1H), 6.80 (d, J=9.1 Hz, 1H), 4.23 (d, J=13.1 Hz, 2H), 3.78-3.73 (m, 1H), 3.62 (s, 4H), 3.50-3.44 (m, 2H), 3.30-3.20 (m, 4H), 3.13-3.08 (m, 1H), 2.96 (d, J=16.0 Hz, 1H), 2.90 (d, J=15.8, 1H), 2.77-2.69 (m, 3H), 2.35-2.29 (m, 1H), 2.28-2.21 (m, 1H), 2.18-2.12 (m, 2H), 1.92-1.87 (m, 2H), 1.77-1.69 (m, 3H), 1.67 (dt, J=9.4, 2.8 Hz, 1H), 1.50-1.39 (m, 3H).

### Example-118 (VP-8-303A-E1, SPV-501)

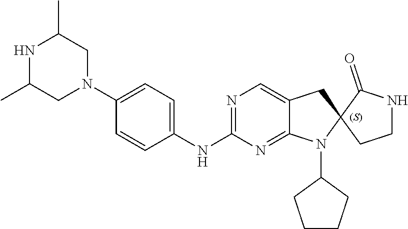

### Synthesis of (3S)-7′-cyclopentyl-2′-((4-(3,5-dimethylpiperazin-1-yl)phenyl)amino)-5′,7′-dihydrospiro[pyrrolidine-3,6′-pyrrolo[2,3-d]pyrimidin]-2-one

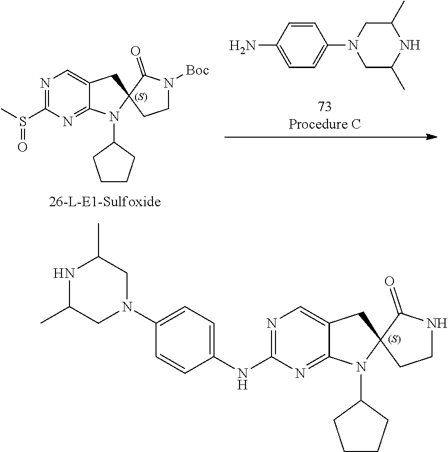

was synthesized following the general procedure C, 6 mg (13% yield). MS(ESI) m/z 462.3 (M+H)⁺. ¹H NMR (600 MHz, DMSO-d₆) δ 8.94 (s, 1H), 8.30 (s, 1H), 8.12 (s, 1H), 7.46 (s, 1H), 7.29 (s, 1H), 6.95 (d, J=8.5 Hz, 2H), 3.78-3.72 (m, 2H), 3.49 (t, J=8.8 Hz, 1H), 3.32 (s, 2H), 3.21-3.17 (m, 1H), 2.98 (d, J=16.9 Hz, 1H), 2.91 (d, J=16.5 Hz, 1H), 2.51 (t, J=12.2 Hz, 2H), 2.37-2.29 (m, 2H), 2.18-2.12 (m, 1H), 2.09 (s, 1H), 2.02-1.97 (m, 1H), 1.67 (d, J=8.7 Hz, 1H), 1.61 (d, J=8.3 Hz, 1H), 1.56 (s, 1H), 1.34 (td, J=16.0, 7.8 Hz, 2H), 1.20 (d, J=6.5 Hz, 6H).

### Example-119 (VP-8-208A-E1, SPV-503)

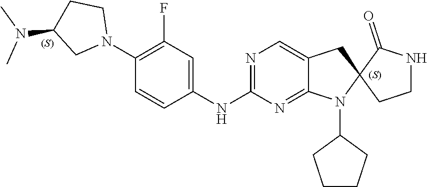

### Synthesis of (S)-7′-cyclopentyl-2′-((4-((S)-3-(dimethylamino)pyrrolidin-1-yl)-3-fluorophenyl)amino)-5′,7′-dihydrospiro[pyrrolidine-3,6′-pyrrolo[2,3-d]pyrimidin]-2-one

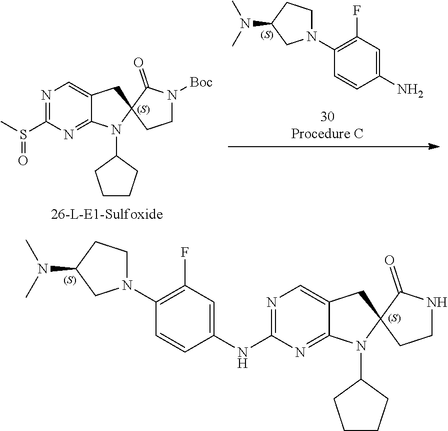

was synthesized following the general procedure C, 7 mg (15% yield). MS(ESI) m/z 480.3 (M+H)⁺. ¹H NMR (600 MHz, DMSO-d₆) δ 8.87 (s, 1H), 8.01 (s, 1H), 7.75 (d, J=16.4 Hz, 1H), 7.59 (s, 1H), 7.22 (d, J=8.6 Hz, 1H), 6.75 (t, J=9.5 Hz, 1H), 3.52-3.45 (m, 1H), 3.39 (s, 2H), 3.29-3.20 (m, 3H), 2.97 (dd, J=15.8, 1.2 Hz, 1H), 2.91 (dd, J=15.7, 1.5 Hz, 1H), 2.85-2.77 (m, 1H), 2.68 (s, 3H), 2.63-2.59 (m, 1H), 2.41-2.29 (m, 3H), 2.29-2.20 (m, 2H), 2.18-2.11 (m, 2H), 2.01 (s, 2H), 1.87-1.80 (m, 2H), 1.76-1.65 (m, 2H), 1.56-1.44 (m, 2H).

### Example-120 (VP-8-211A-E1, SPV-504)

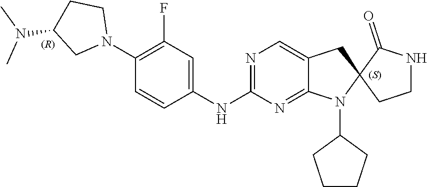

### Synthesis of (S)-7′-cyclopentyl-2′-((4-((R)-3-(dimethylamino)pyrrolidin-1-yl)-3-fluorophenyl)amino)-5′,7′-dihydrospiro[pyrrolidine-3,6′-pyrrolo[2,3-d]pyrimidin]-2-one

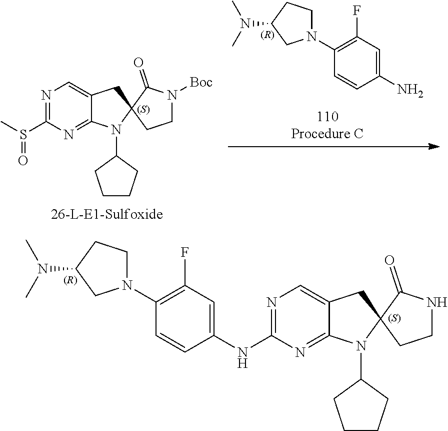

was synthesized following the general procedure C, 6 mg (13% yield). MS(ESI) m/z 480.3 (M+H)⁺. ¹H NMR (600 MHz, DMSO-d₆) δ 7.99 (d, J=17.9 Hz, 2H), 7.51 (s, 1H), 7.12 (d, J=8.7 Hz, 1H), 6.73 (t, J=9.5 Hz, 1H), 6.46 (s, 1H), 3.48-3.32 (m, 4H), 3.22-3.14 (m, 3H), 2.93 (d, J=16.3 Hz, 1H), 2.87 (d, J=16.3 Hz, 1H), 2.75 (s, 6H), 2.33-2.23 (m, 3H), 2.16-2.07 (m, 2H), 2.07-1.99 (m, 2H), 1.71 (s, 1H), 1.69-1.60 (m, 2H), 1.48-1.36 (m, 3H).

### Example-121 (VP-8-213C-E1, SPV-505)

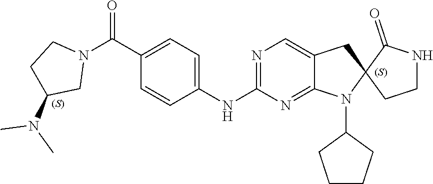

### Synthesis of (S)-7′-cyclopentyl-2′-((4-((S)-3-(dimethylamino)pyrrolidine-1-carbonyl)phenyl)amino)-5′,7′-dihydrospiro[pyrrolidine-3,6′-pyrrolo[2,3-d]pyrimidin]-2-one

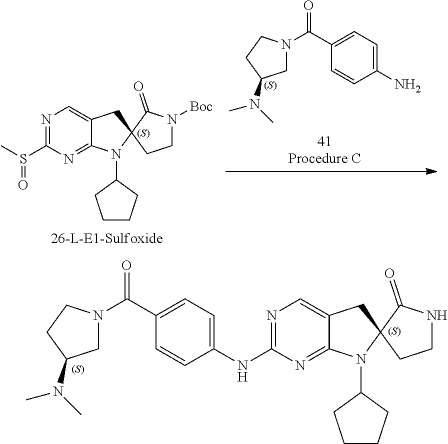

was synthesized following the general procedure C, 6 mg (12% yield). MS(ESI) m/z 490.3 (M+H)⁺. ¹H NMR (600 MHz, DMSO-d₆) δ 7.95 (s, 1H), 7.71 (d, J=8.3 Hz, 2H), 7.57 (s, 1H), 7.35 (s, 2H), 3.66-3.37 (m, 1H), 3.21-3.03 (m, 4H), 2.93 (d, J=16.6 Hz, 2H), 2.87 (d, J=16.1 Hz, 2H), 2.66-2.52 (m, 1H), 2.43 (s, 6H), 2.35-2.16 (m, 2H), 2.09 (dd, J=13.0, 6.6 Hz, 1H), 2.05 (s, 2H), 1.78 (d, J=7.1 Hz, 2H), 1.65 (d, J=11.0 Hz, 3H), 1.46 (dq, J=23.3, 7.9 Hz, 1H), 1.10-1.04 (m, 1H).

### Example-122 (VP-8-216D-E1, SPV-506)

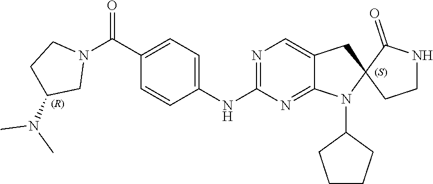

### Synthesis of (S)-7′-cyclopentyl-2′-((4-((R)-3-(dimethylamino)pyrrolidine-1-carbonyl)phenyl)amino)-5′,7′-dihydrospiro[pyrrolidine-3,6′-pyrrolo[2,3-d]pyrimidin]-2-one

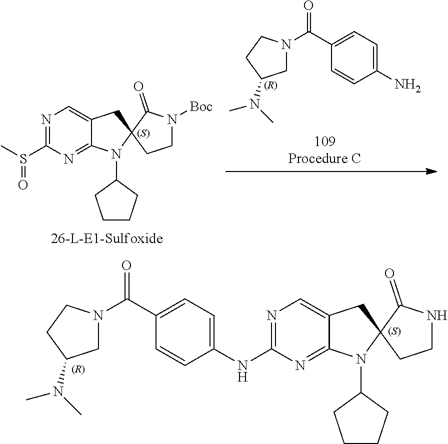

was synthesized following the general procedure C, 4 mg (8% yield). MS(ESI) m/z 490.3 (M+H)⁺.

### Example-123 (VP-8-185B-E1, SPV-507)

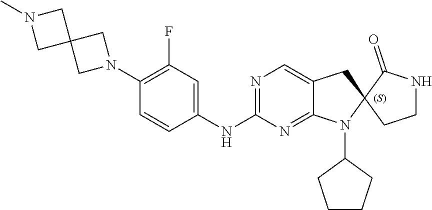

### Synthesis of (S)-7′-cyclopentyl-2′-((3-fluoro-4-(6-methyl-2,6-diazaspiro[3.3]heptan-2-yl)phenyl)amino)-5′,7′-dihydrospiro[pyrrolidine-3,6′-pyrrolo[2,3-d]pyrimidin]-2-one

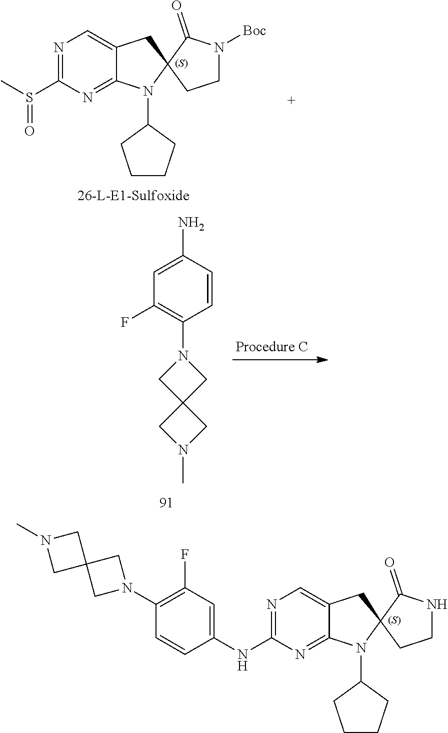

was synthesized following the general procedure C, 2 mg (4% yield). MS(ESI) m/z 478.3 (M+H)⁺. ¹H NMR (600 MHz, DMSO-d₆) δ 8.58 (s, 1H), 7.93 (s, 1H), 7.59 (dd, J=14.8, 2.4 Hz, 1H), 7.50 (d, J=1.5 Hz, 1H), 7.06 (dd, J=8.6, 2.4 Hz, 1H), 6.59 (t, J=9.4 Hz, 1H), 3.49 (s, 1H), 3.39 (p, J=9.0 Hz, 2H), 3.17 (ddd, J=18.7, 9.9, 2.3 Hz, 3H), 3.13 (d, J=5.7 Hz, 2H), 2.88 (d, J=15.8 Hz, 1H), 2.83 (d, J=16.7 Hz, 1H), 2.44 (s, 5H), 2.31-2.15 (m, 4H), 2.07 (ddd, J=13.1, 6.8, 2.1 Hz, 1H), 1.81-1.72 (m, 2H), 1.68-1.60 (m, 2H), 1.50-1.36 (m, 2H).

### Example-124 (VP-8-188B-E1, SPV-508)

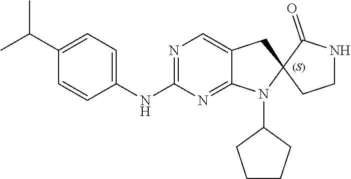

### Synthesis of (S)-7′-cyclopentyl-2′-((4-isopropylphenyl)amino)-5′,7′-dihydrospiro[pyrrolidine-3,6′-pyrrolo[2,3-d]pyrimidin]-2-one

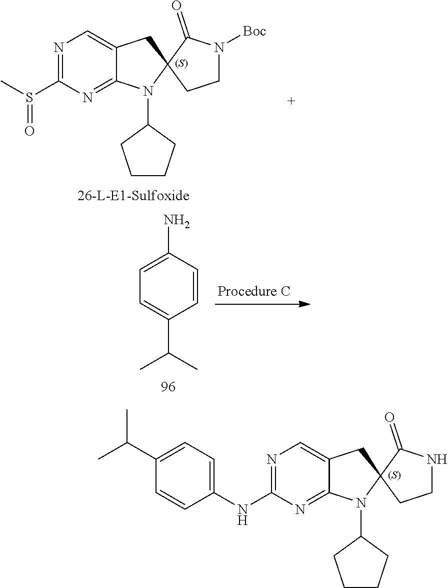

was synthesized following the general procedure C, 8 mg (20% yield). MS(ESI) m/z 392.3 (M+H)⁺. ¹H NMR (600 MHz, DMSO-d₆) δ 8.16 (s, 1H), 7.55 (s, 1H), 7.41 (d, J=8.1 Hz, 2H), 7.21 (d, J=8.1 Hz, 2H), 3.54 (p, J=8.9 Hz, 1H), 3.31-3.21 (m, 3H), 3.04 (d, J=16.6 Hz, 1H), 2.97 (d, J=17.1 Hz, 1H), 2.87 (hept, J=6.8 Hz, 1H), 2.42-2.35 (m, 1H), 2.25-2.14 (m, 2H), 2.14-2.05 (m, 1H), 1.78-1.71 (m, 1H), 1.71-1.58 (m, 2H), 1.51-1.35 (m, 2H), 1.19 (d, J=6.9 Hz, 6H).

### Example-125 (VP-8-198A-E1, SPV-509)

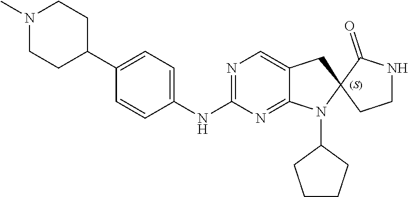

### Synthesis of (S)-7′-cyclopentyl-2′-((4-(1-methylpiperidin-4-yl)phenyl)amino)-5′,7′-dihydrospiro[pyrrolidine-3,6′-pyrrolo[2,3-d]pyrimidin]-2-one

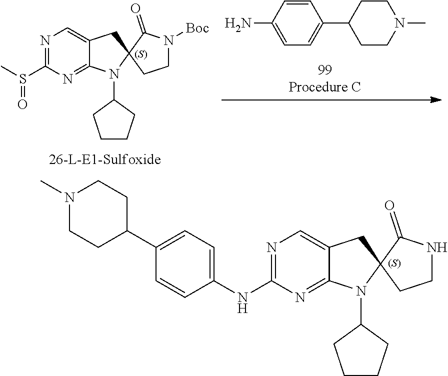

was synthesized following the general procedure C, 7 mg (16% yield). MS(ESI) m/z 447.3 (M+H)⁺. ¹H NMR (600 MHz, DMSO-d₆) δ 8.85 (s, 1H), 8.01 (s, 1H), 7.67 (d, J=8.6 Hz, 2H), 7.60 (s, 1H), 7.08 (d, J=8.5 Hz, 2H), 3.52-3.45 (m, 1H), 3.40 (d, J=11.3 Hz, 2H), 3.28-3.21 (m, 3H), 2.97 (d, J=16.4 Hz, 1H), 2.91 (d, J=15.7 Hz, 1H), 2.74 (s, 3H), 2.71-2.63 (m, 1H), 2.40-2.29 (m, 3H), 2.29-2.21 (m, 1H), 2.18-2.11 (m, 1H), 1.94 (d, J=13.9 Hz, 2H), 1.87-1.75 (m, 3H), 1.75-1.65 (m, 2H), 1.58-1.44 (m, 3H).

### Example-126 (VP-8-199B-E1, SPV-510)

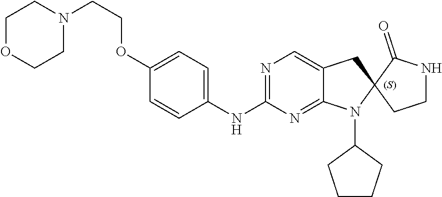

### Synthesis of (S)-7′-cyclopentyl-2′-((4-(2-morpholinoethoxy)phenyl)amino)-5′,7′-dihydrospiro[pyrrolidine-3,6′-pyrrolo[2,3-d]pyrimidin]-2-one

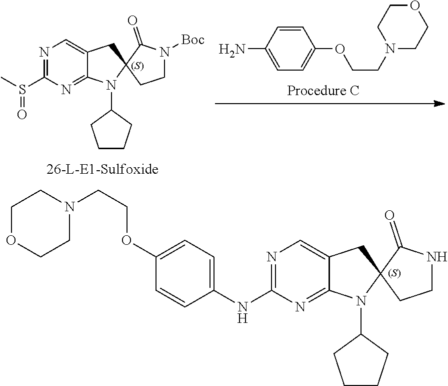

was synthesized following the general procedure C, 5 mg (10% yield). MS(ESI) m/z 479.3 (M+H)⁺. ¹H NMR (600 MHz, DMSO-d₆) δ 8.06 (s, 1H), 7.55 (s, 1H), 7.51 (d, J=8.3 Hz, 2H), 6.88 (d, J=8.5 Hz, 2H), 4.09 (s, 2H), 3.62 (s, 3H), 3.55-3.45 (m, 1H), 3.29-3.19 (m, 3H), 2.99 (d, J=16.3 Hz, 1H), 2.93 (d, J=17.0 Hz, 1H), 2.61 (p, J=1.9 Hz, 1H), 2.50 (s, 3H), 2.41-2.31 (m, 2H), 2.31-2.21 (m, 2H), 2.21-2.12 (m, 2H), 1.80-1.63 (m, 4H), 1.54-1.39 (m, 2H).

### Example-127 (VP-8-202D-E1, SPV 512)

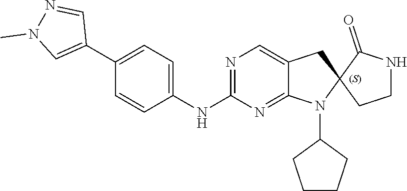

### Synthesis of (S)-7′-cyclopentyl-2′-((4-(1-methyl-1H-pyrazol-4-yl)phenyl)amino)-5′,7′-dihydrospiro[pyrrolidine-3,6′-pyrrolo[2,3-d]pyrimidin]-2-one

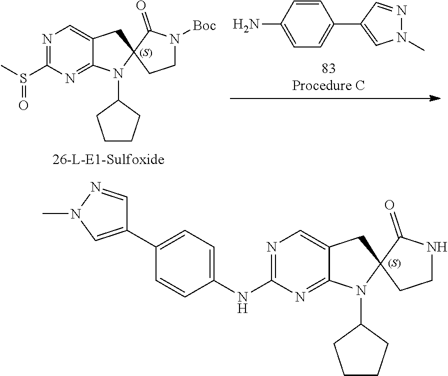

was synthesized following the general procedure C, 12 mg (28% yield). MS(ESI) m/z 430.3 (M+H)⁺. ¹H NMR (600 MHz, DMSO-d₆) δ 8.92 (s, 1H), 8.01 (s, 2H), 7.76 (s, 1H), 7.69 (d, J=8.6 Hz, 2H), 7.61 (s, 1H), 7.40 (d, J=8.6 Hz, 2H), 3.84 (s, 3H), 3.50 (p, J=9.1 Hz, 1H), 3.28-3.20 (m, 2H), 2.98 (d, J=16.1 Hz, 1H), 2.92 (d, J=16.8 Hz, 1H), 2.41-2.30 (m, 2H), 2.30-2.23 (m, 1H), 2.19-2.13 (m, 1H), 1.90-1.80 (m, 2H), 1.77-1.68 (m, 2H), 1.58-1.46 (m, 2H).

### Example-128 (VP-8-204A-E1, SPV-513)

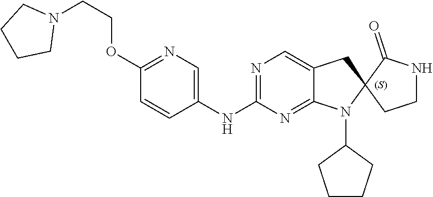

### Synthesis of (S)-7′-cyclopentyl-2′-((6-(2-(pyrrolidin-1-yl)ethoxy)pyridin-3-yl)amino)-5′,7′-dihydrospiro[pyrrolidine-3,6′-pyrrolo[2,3-d]pyrimidin]-2-one

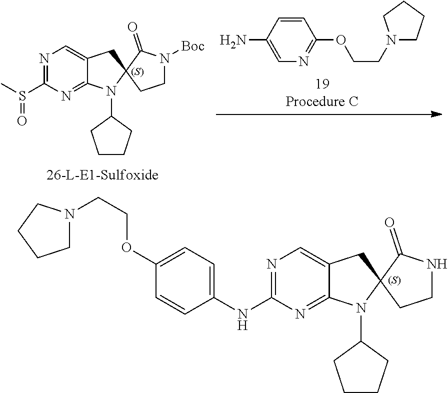

was synthesized following the general procedure C, 4 mg (9% yield). MS(ESI) m/z 464.3 (M+H)⁺.

### Example-129 (VP-NR11-142B-E1, SPV-515)

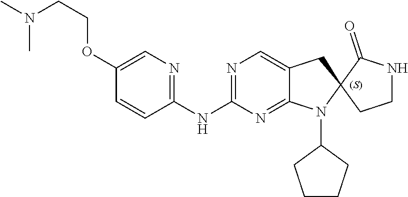

### Synthesis of (S)-7′-cyclopentyl-2′-((5-(2-(dimethylamino)ethoxy)pyridin-2-yl)amino)-5′,7′-dihydrospiro[pyrrolidine-3,6′-pyrrolo[2,3-d]pyrimidin]-2-one

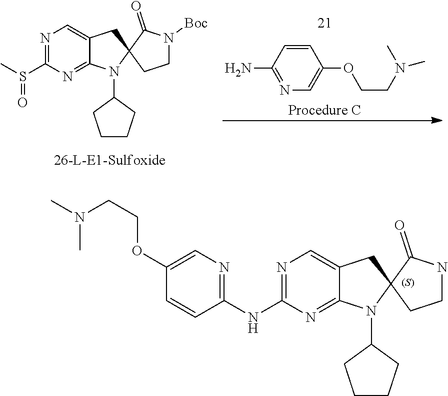

was synthesized following the general procedure C, 2 mg (5% yield). MS(ESI) m/z 438.3 (M+H)⁺. ¹H NMR (600 MHz, DMSO-d₆) δ 8.59 (s, 1H), 8.10 (d, J=9.1 Hz, 1H), 8.02 (s, 1H), 7.95 (d, J=3.1 Hz, 1H), 7.62 (d, J=1.4 Hz, 1H), 7.35 (dd, J=9.1, 3.1 Hz, 1H), 4.07 (t, J=5.8 Hz, 2H), 3.48 (p, J=9.0 Hz, 1H), 3.28-3.20 (m, 2H), 2.99 (d, J=16.2 Hz, 1H), 2.93 (d, J=15.7 Hz, 1H), 2.63-2.57 (m, 2H), 2.50 (s, 3H), 2.38 (p, J=1.9 Hz, 1H), 2.37-2.29 (m, 2H), 2.21 (s, 3H), 2.18-2.13 (m, 1H), 1.88-1.81 (m, 2H), 1.74-1.67 (m, 2H), 1.54-1.45 (m, 2H).

### Example-130 (VP-NR10-108A, SPV-439)

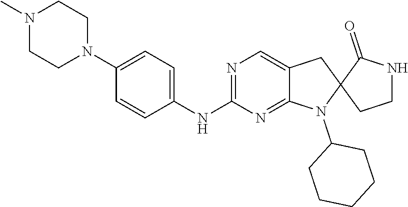 [SMILES: CN1CCN(c2ccc(Nc3ncc4c(n3)N(C3CCCCC3)C3(CCNC3=O)C4)cc2)CC1]

### Step 1: Synthesis of ethyl 2-(cyclohexylamino)acetate hydrochloride (130-C)

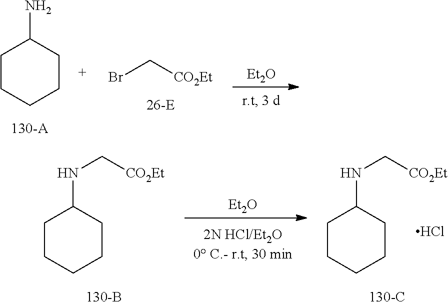

Synthesis of 130-B: (Ref: WO 199948858): Prepared using the procedure described for the synthesis of compound 26-F.

Synthesis of 130-C: Prepared using the procedure described for the synthesis of compound 26-G. MS(ESI) m/z 186.1 [M+H]⁺.

(5.5 g, 87% yield)

### Step 2: Synthesis of 7-cyclohexyl-2-(methylthio)-7H-pyrrolo[2,3-d]pyrimidine-6-carboxylic Acid (130-E)

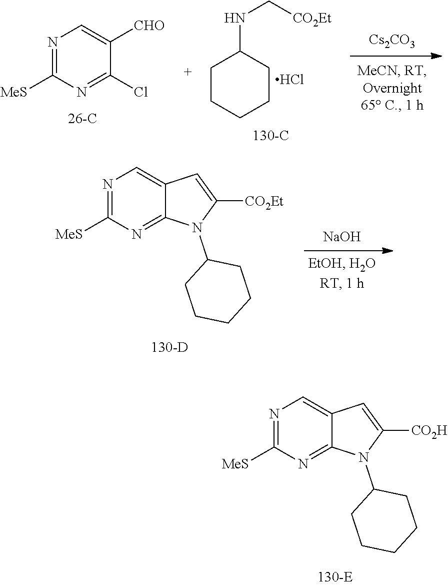

Synthesis of 130-D: Prepared using the procedure described for the synthesis of compound 26-H. (used as such for next step). MS(ESI) m/z 320.1 [M+H]⁺. ¹H NMR (600 MHz, Chloroform-d) δ 8.80 (s, 1H), 7.21 (s, 1H), 5.30 (tt, J=12.1, 3.9 Hz, 1H), 4.37 (q, J=7.1 Hz, 2H), 2.72 (qd, J=12.4, 3.2 Hz, 2H), 2.63 (s, 3H), 1.96-1.84 (m, 4H), 1.76 (d, J=13.4 Hz, 1H), 1.51-1.37 (m, 5H), 1.37-1.24 (m, 1H).

Synthesis of 130-E: Prepared using the procedure described for the synthesis of compound 26-I. (12.5 g, 81% yield, over two steps). MS(ESI) m/z 278.1 [M+H]⁺. ¹H NMR (600 MHz, DMSO-d₆) δ 8.94 (s, 1H), 7.26 (s, 1H), 5.30 (tt, J=12.2, 3.9 Hz, 1H), 2.70-2.60 (m, 2H), 2.58 (s, 3H), 1.93-1.66 (m, 5H), 1.41-1.20 (m, 3H).

### Step 3: Synthesis of tert-butyl 7′-cyclohexyl-2′-(methylsulfinyl)-2-oxo-5′,7′-dihydrospiro[pyrrolidine-3,6′-pyrrolo[2,3-d]pyrimidine]-1-carboxylate (130-I)

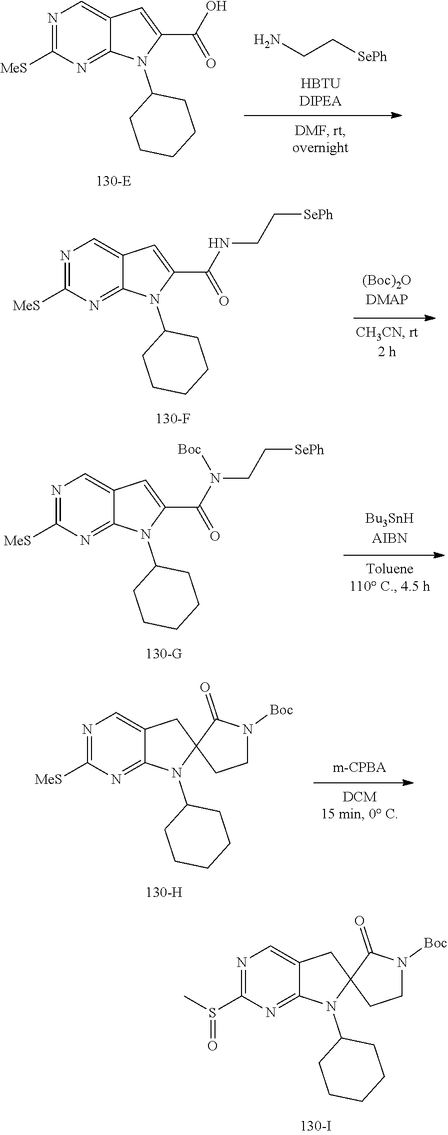

Synthesis of 130-F: Prepared using the procedure described for the synthesis of compound 1-D.

(used as such for next step). MS(ESI) m/z 475.2 (M+H)⁺. ¹H NMR (600 MHz, Chloroform-d) δ 8.74 (s, 1H), 7.62-7.53 (m, 2H), 7.29 (dd, J=5.3, 1.9 Hz, 3H), 6.52 (s, 2H), 4.96 (tt, J=12.6, 3.7 Hz, 1H), 3.77-3.66 (m, 2H), 3.16 (t, J=6.4 Hz, 2H), 2.72-2.65 (m, 2H), 2.63 (s, 3H), 1.89 (d, J=11.7 Hz, 4H), 1.74 (d, J=13.2 Hz, 1H), 1.48-1.38 (m, 2H), 1.34-1.24 (m, 1H).

Synthesis of 130-G: Prepared using the procedure described for the synthesis of compound 1-E.

(19 g, 77% yield, over two steps). MS(ESI) m/z 575.2 (M+H)⁺.

Synthesis of 130-H: Prepared using the procedure described for the synthesis of compound 1-F and 1-G. (7.4 g, 54% yield). MS(ESI) m/z 419.1 (M+H)⁺. ¹H NMR (600 MHz, Chloroform-d) δ 7.74-7.65 (m, 1H), 3.98-3.84 (m, 1H), 3.57 (td, J=10.8, 6.3 Hz, 1H), 3.18 (d, 1H), 2.99-2.86 (m, 2H), 2.50 (s, 5H), 2.36-2.27 (m, 1H), 2.09-2.03 (m, 1H), 2.00 (d, J=12.8 Hz, 1H), 1.88-1.78 (m, 2H), 1.56 (s, 9H), 1.29-1.14 (m, 4H).

Synthesis of 130-I: Prepared using the procedure described for the synthesis of compound 26-M.

MS(ESI) m/z 435.2 (M+H)⁺.

### Step 4: Synthesis of 7′-cyclohexyl-2′-((4-(4-methylpiperazin-1-yl)phenyl)amino)-5′,7′-dihydrospiro[pyrrolidine-3,6′-pyrrolo[2,3-d]pyrimidin]-2-one

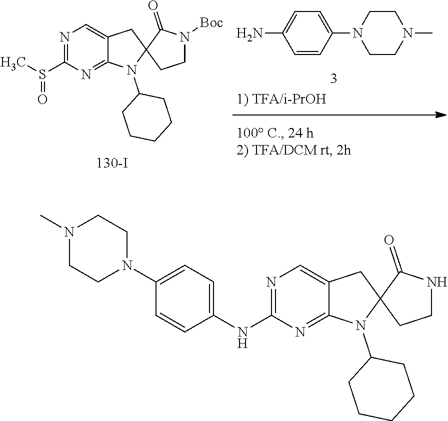 [SMILES: CS(=O)c1ncc2c(n1)N(C1CCCCC1)C1(CCN(C(=O)OC(C)(C)C)C1=O)C2 ; CN1CCN(c2ccc(N)cc2)CC1 ; CN1CCN(c2ccc(Nc3ncc4c(n3)N(C3CCCCC3)C3(CCNC3=O)C4)cc2)CC1]

was synthesized following the general procedure C, 2 mg (4% yield). MS(ESI) m/z 462.3 (M+H)⁺.

### Example-131 (VP-NR10-109A, SPV-440)

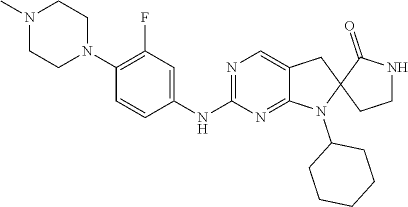 [SMILES: CN1CCN(c2ccc(Nc3ncc4c(n3)N(C3CCCCC3)C3(CCNC3=O)C4)cc2F)CC1]

### Synthesis of 7′-cyclohexyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one

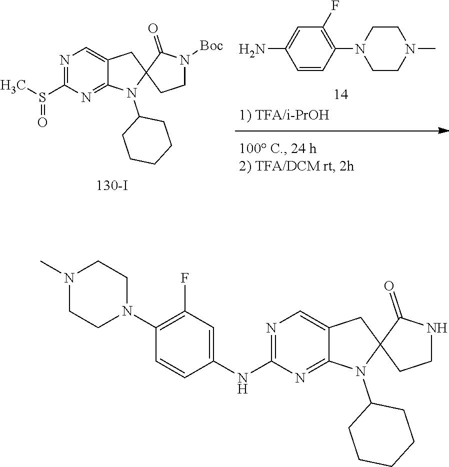 [SMILES: CS(=O)c1ncc2c(n1)N(C1CCCCC1)C1(CCN(C(=O)OC(C)(C)C)C1=O)C2 ; CN1CCN(c2ccc(N)cc2F)CC1 ; CN1CCN(c2ccc(Nc3ncc4c(n3)N(C3CCCCC3)C3(CCNC3=O)C4)cc2F)CC1]

was synthesized following the general procedure C, 19 mg (40% yield). MS(ESI) m/z 480.3 (M+H)⁺. ¹H NMR (600 MHz, DMSO-d₆) δ 8.98 (s, 1H), 7.95 (s, 1H), 7.84 (dd, J=15.7, 2.4 Hz, 1H), 7.52 (d, J=1.4 Hz, 1H), 7.20 (dd, J=8.8, 2.4 Hz, 1H), 6.92-6.86 (m, 1H), 3.21-3.13 (m, 3H), 3.05-2.99 (m, 3H), 2.98 (s, 3H), 2.94-2.88 (m, 2H), 2.88-2.82 (m, 2H), 2.56 (d, J=9.3 Hz, 3H), 2.40-2.22 (m, 3H), 2.09-2.03 (m, 1H), 1.81-1.76 (m, 1H), 1.69 (d, J=12.0 Hz, 2H), 1.55 (d, J=11.5 Hz, 2H), 1.28-1.10 (m, 3H).

### Example-132 (VP-NR10-110A, SPV-441)

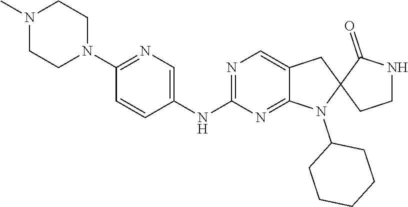 [SMILES: CN1CCN(c2ccc(Nc3ncc4c(n3)N(C3CCCCC3)C3(CCNC3=O)C4)cn2)CC1]

### Synthesis of 7′-cyclohexyl-2′-((6-(4-methylpiperazin-1-yl)pyridin-3-yl)amino)-5′,7′-dihydrospiro[pyrrolidine-3,6′-pyrrolo[2,3-d]pyrimidin]-2-one

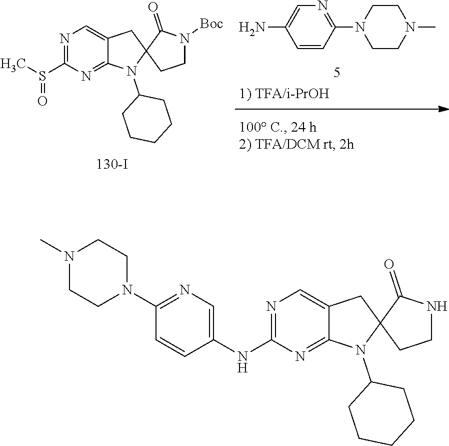 [SMILES: CS(=O)c1ncc2c(n1)N(C1CCCCC1)C1(CCN(C(=O)OC(C)(C)C)C1=O)C2 ; CN1CCN(c2ccc(N)cn2)CC1 ; CN1CCN(c2ccc(Nc3ncc4c(n3)N(C3CCCCC3)C3(CCNC3=O)C4)cn2)CC1]

was synthesized following the general procedure C, 15 mg (32% yield). MS(ESI) m/z 463.3 (M+H)⁺. ¹H NMR (600 MHz, DMSO-d₆) δ 8.63 (s, 1H), 8.37 (d, J=2.7 Hz, 1H), 7.93 (s, 1H), 7.85 (dd, J=9.0, 2.7 Hz, 1H), 7.47 (d, J=1.6 Hz, 1H), 6.73 (d, J=9.1 Hz, 1H), 3.21-3.13 (m, 3H), 2.93-2.80 (m, 3H), 2.65 (s, 4H), 2.37 (s, 2H), 2.36-2.34 (m, 1H), 2.34-2.29 (m, 1H), 2.29-2.26 (m, 1H), 2.26-2.21 (m, 1H), 2.08-2.02 (m, 1H), 1.84 (s, 3H), 1.76-1.71 (m, 1H), 1.67 (dd, J=9.2, 5.8 Hz, 2H), 1.55-1.48 (m, 2H), 1.26-1.16 (m, 1H), 1.16-1.06 (m, 2H).

### Example-133 (VP-NR10-124A, SPV-442)

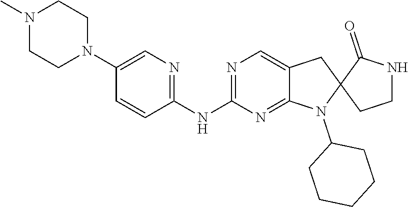 [SMILES: CN1CCN(c2ccc(Nc3ncc4c(n3)N(C3CCCCC3)C3(CCNC3=O)C4)nc2)CC1]

### Synthesis of 7′-cyclohexyl-2′-((5-(4-methylpiperazin-1-yl)pyridin-2-yl)amino)-5′,7′-dihydrospiro[pyrrolidine-3,6′-pyrrolo[2,3-d]pyrimidin]-2-one

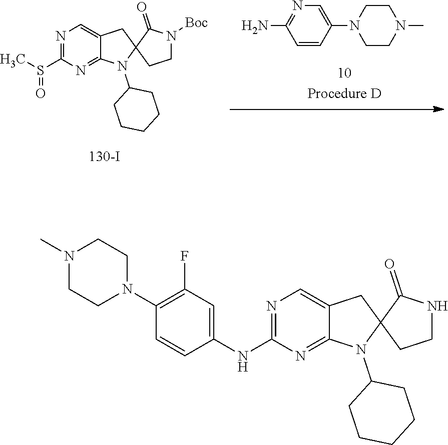 [SMILES: CS(=O)c1ncc2c(n1)N(C1CCCCC1)C1(CCN(C(=O)OC(C)(C)C)C1=O)C2 ; CN1CCN(c2ccc(N)nc2)CC1 ; CN1CCN(c2ccc(Nc3ncc4c(n3)N(C3CCCCC3)C3(CCNC3=O)C4)cc2F)CC1]

was synthesized following the general procedure C, 6 mg (13% yield). MS(ESI) m/z 463.3 (M+H)⁺. ¹H NMR (600 MHz, DMSO-d₆) δ 8.18 (s, 1H), 7.94 (s, 1H), 7.62 (s, 1H), 7.60 (s, 1H), 7.29 (s, 1H), 3.07 (d, J=17.0 Hz, 3H), 2.99 (d, J=17.1 Hz, 2H), 2.70 (s, 4H), 2.55 (s, 1H), 2.44 (s, 3H), 2.34-2.27 (m, 3H), 2.18 (dd, J=13.6, 6.7 Hz, 1H), 1.77 (d, J=12.1 Hz, 1H), 1.71 (d, J=11.2 Hz, 3H), 1.62-1.54 (m, 3H), 1.29 (d, J=13.1 Hz, 1H), 1.19-1.12 (m, 3H).

### Separation of two enantiomers of tert-butyl 7′-cyclohexyl-2′-(methylthio)-2-oxo-5′,7′-dihydrospiro[pyrrolidine-3,6′-pyrrolo[2,3-d]pyrimidine]-1-carboxylate (130-H)

### Analytical Chiral Column Chromatography

### Column: ChiralPak OZ-H 4.6×250 mm 5 μm; Mobile Phase: Acetonitrile

Flow Rate: 1.0 mL/min; wavelengths: 254 nm and 280 nm

### Preparative Chiral Chromatography Conditions

Column: Chiralpak OZ-H 21×250 mm, 5 micron; Mobile phase: Acetonitrile; Flow rate: 3 mL/min; Wavelength: 254 nm

### Retention Times (t_{R}): 5.00 min. (Enantiomer-1, E1), 6.60 min. (Enantiomer-2, E2)

Standard chromatographic techniques were used to isolate the required two enantiomers, E1 and E2 as off-white solids (concentrated and lyophilized).

About 1.4 g of racemic 130-H was used to isolate the following amounts:

Enantiomer-1 (E1): Yield: 0.6 g (43%), Purity: >98%; Optical purity: % ee >96%
Enantiomer-2 (E2): Yield: 0.6 g (43%); Purity: >98%; Optical purity: % ee >96%

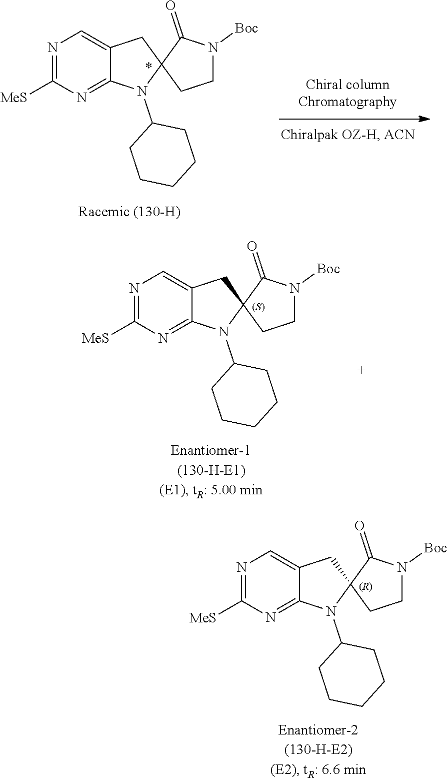

### Synthesis of tert-butyl 7′-cyclohexyl-2′-(methylthio)-2-oxo-5′,7′-dihydrospiro[pyrrolidine-3,6′-pyrrolo[2,3-d]pyrimidine]-1-carboxylate (130-H-E1sulfoxide)

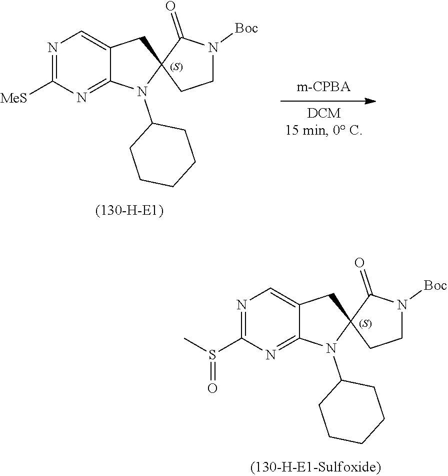

E1-sulfoxide (diastereomers) was synthesized according to the procedure described for making racemic sulfoxide 130-I. Yield: 600 mg (97%)

MS(ESI) m/z 421.2 (M+H)⁺. ¹H NMR (600 MHz, Chloroform-d) δ 7.68 (d, J=1.4 Hz, 1H), 3.89 (ddd, J=11.1, 9.1, 1.1 Hz, 1H), 3.57 (td, J=10.9, 6.5 Hz, 1H), 3.18 (dd, J=16.1, 1.5 Hz, 1H), 2.96-2.88 (m, 2H), 2.50 (s, 5H), 2.30 (ddd, J=12.9, 10.7, 9.0 Hz, 1H), 2.06 (ddd, J=12.9, 6.4, 1.0 Hz, 1H), 2.00 (d, J=11.0 Hz, 1H), 1.84-1.79 (m, 2H), 1.68-1.61 (m, 1H), 1.56 (s, 10H), 1.22 (dddd, J=24.5, 16.0, 12.9, 9.7 Hz, 3H).

### Synthesis of tert-butyl 7′-cyclohexyl-2′-(methylsulfinyl)-2-oxo-5′,7′-dihydrospiro[pyrrolidine-3,6′-pyrrolo[2,3-d]pyrimidine]-1-carboxylate (121-I-E2sulfoxide)

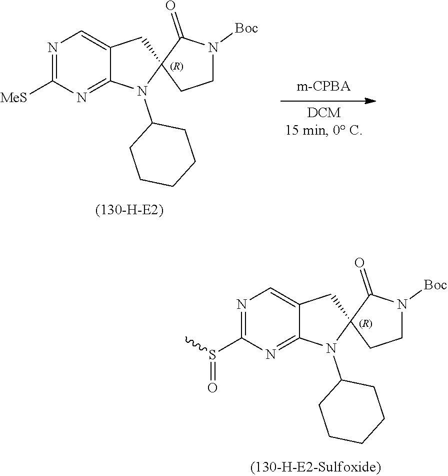

E2-sulfoxide (diastereomers) was synthesized according to the procedure described for making racemic sulfoxide 130-I. Yield: 590 mg (95%)

MS(ESI) m/z 421.2 (M+H)⁺.

### Example-134 (VP-8-273A-E1, SPV-489)

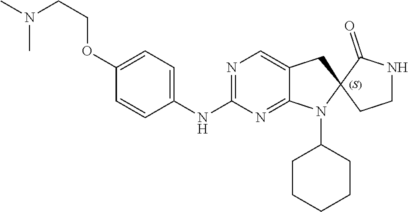

### Synthesis of (S)-7′-cyclohexyl-2′-((4-(2-(dimethylamino)ethoxy)phenyl)amino)-5′,7′-dihydrospiro[pyrrolidine-3,6′-pyrrolo[2,3-d]pyrimidin]-2-one

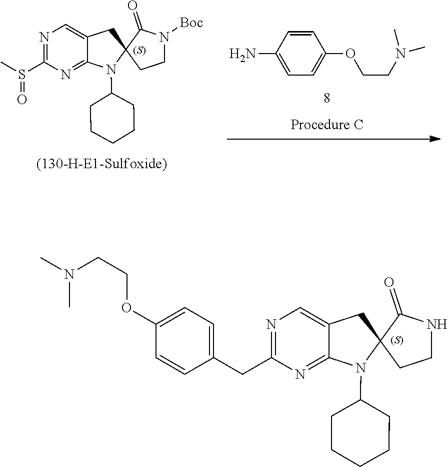

was synthesized following the general procedure C, 8 mg (18% yield). MS(ESI) m/z 451.3 (M+H)⁺.

### Separation of two enantiomers of tert-butyl 2′-chloro-7′-cyclopentyl-2-oxo-5′,7′-dihydrospiro[pyrrolidine-3,6′-pyrrolo[2,3-d]pyrimidine]-1-carboxylate (33-F)

### Analytical Chiral Column Chromatography

### Column: ChiralPak OZ-H 4.6×250 mm 5 μm; Mobile Phase: Acetonitrile

Flow Rate: 1.0 mL/min; wavelengths: 254 nm and 280 nm

### Preparative Chiral Chromatography Conditions

Column: Chiralpak OZ-H 21×250 mm, 5 micron; Mobile phase: Acetonitrile; Flow rate: 3 mL/min; Wavelength: 254 nm

### Retention Times (t_{R}): 5.50 min. (Enantiomer-1, E1), 7.17 min. (Enantiomer-2, E2)

Standard chromatographic techniques were used to isolate the required two enantiomers, E1 and E2 as off-white solids (concentrated and lyophilized).

About 1 g of racemic 33-F was used to isolate the following amounts:

Enantiomer-1 (E1): Yield: 0.43 g (43%), Purity: >98%; Optical purity: % ee >98%
Enantiomer-2 (E2): Yield: 0.42 g (42%); Purity: >98%; Optical purity: % ee >98%

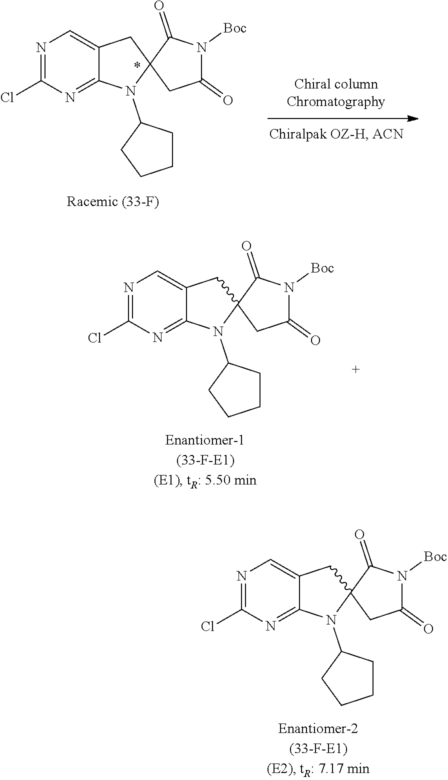 [SMILES: CC(C)(C)OC(=O)N1C(=O)CC2(Cc3cnc(Cl)nc3N2C2CCCC2)C1=O ; CC(C)(C)OC(=O)N1C(=O)CC2(Cc3cnc(Cl)nc3N2C2CCCC2)C1=O ; CC(C)(C)OC(=O)N1C(=O)CC2(Cc3cnc(Cl)nc3N2C2CCCC2)C1=O]

### Example-135 (VP-8-9A-E1, SPV-498)

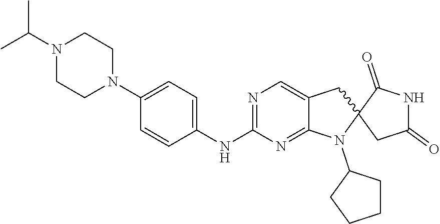 [SMILES: CC(C)N1CCN(c2ccc(Nc3ncc4c(n3)N(C3CCCC3)C3(CC(=O)NC3=O)C4)cc2)CC1]

### Synthesis of 7′-cyclopentyl-2′-((4-(4-isopropylpiperazin-1-yl)phenyl)amino)-5′,7′-dihydrospiro[pyrrolidine-3,6′-pyrrolo[2,3-d]pyrimidine]-2,5-dione

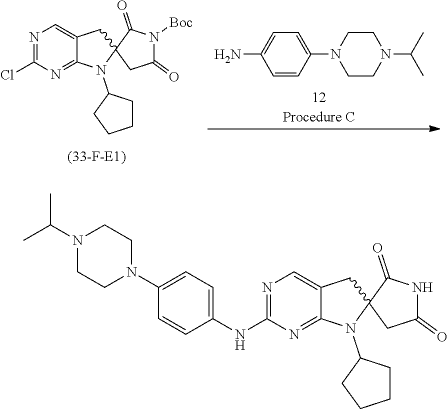 [SMILES: CC(C)(C)OC(=O)N1C(=O)CC2(Cc3cnc(Cl)nc3N2C2CCCC2)C1=O ; CC(C)N1CCN(c2ccc(N)cc2)CC1 ; CC(C)N1CCN(c2ccc(Nc3ncc4c(n3)N(C3CCCC3)C3(CC(=O)NC3=O)C4)cc2)CC1]

was synthesized following the general procedure C, 5 mg (10% yield). MS(ESI) m/z 490.3 (M+H)⁺. ¹H NMR (600 MHz, DMSO-d₆) δ 11.64 (s, 1H), 9.32 (s, 1H), 7.61 (s, 1H), 7.48 (s, 1H), 6.96 (d, J=8.5 Hz, 2H), 3.77 (s, 2H), 3.62-3.45 (m, 5H), 3.29-3.23 (m, 3H), 3.15 (d, J=16.8 Hz, 3H), 3.07 (s, 1H), 3.03 (s, 1H), 2.92 (s, 1H), 2.17 (s, 1H), 1.80-1.76 (m, 1H), 1.71 (s, 1H), 1.62-1.59 (m, 1H), 1.44 (s, 2H), 1.29 (d, J=6.6 Hz, 6H).

### Example-136 (VP-8-11A-E1, SPV-499)

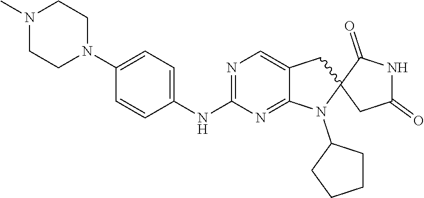 [SMILES: CN1CCN(c2ccc(Nc3ncc4c(n3)N(C3CCCC3)C3(CC(=O)NC3=O)C4)cc2)CC1]

### Synthesis of 7′-cyclopentyl-2′-((4-(4-methylpiperazin-1-yl)phenyl)amino)-5′,7′-dihydrospiro[pyrrolidine-3,6′-pyrrolo[2,3-d]pyrimidine]-2,5-dione

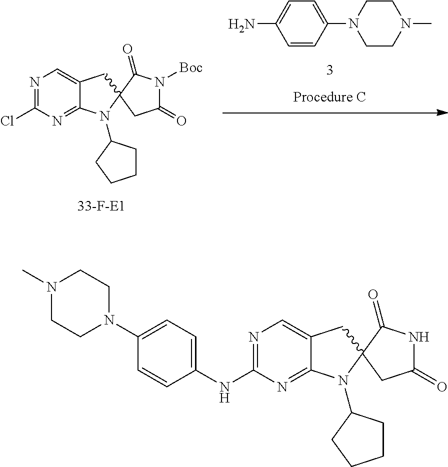 [SMILES: CN1CCN(c2ccc(N)cc2)CC1 ; CC(C)(C)OC(=O)N1C(=O)CC2(Cc3cnc(Cl)nc3N2C2CCCC2)C1=O ; CN1CCN(c2ccc(Nc3ncc4c(n3)N(C3CCCC3)C3(CC(=O)NC3=O)C4)cc2)CC1]

was synthesized following the general procedure C, 11 mg (24% yield). MS(ESI) m/z 462.3 (M+H)⁺.

### Example-137 (VP-8-304A-E1, SPV-502)

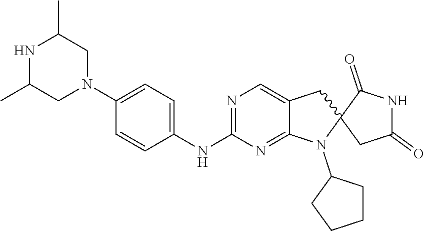 [SMILES: CC1CN(c2ccc(Nc3ncc4c(n3)N(C3CCCC3)C3(CC(=O)NC3=O)C4)cc2)CC(C)N1]

### Synthesis of 7′-cyclopentyl-2′-((4-(3,5-dimethylpiperazin-1-yl)phenyl)amino)-5′,7′-dihydrospiro[pyrrolidine-3,6′-pyrrolo[2,3-d]pyrimidine]-2,5-dione

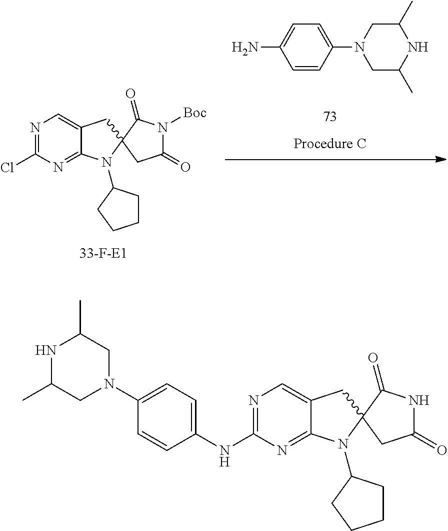 [SMILES: CC1CN(c2ccc(N)cc2)CC(C)N1 ; CC(C)(C)OC(=O)N1C(=O)CC2(Cc3cnc(Cl)nc3N2C2CCCC2)C1=O ; CC1CN(c2ccc(Nc3ncc4c(n3)N(C3CCCC3)C3(CC(=O)NC3=O)C4)cc2)CC(C)N1]

was synthesized following the general procedure C, 6 mg (13% yield). MS(ESI) m/z 476.3 (M+H)⁺. ¹H NMR (600 MHz, DMSO-d₆) δ 8.55 (s, 1H), 7.53 (d, J=1.4 Hz, 1H), 7.45-7.41 (m, 2H), 6.75-6.71 (m, 2H), 3.48-3.41 (m, 2H), 3.17-3.12 (m, 3H), 3.06-2.97 (m, 2H), 2.96 (s, 1H), 2.91 (s, 1H), 2.83-2.75 (m, 3H), 2.21-2.13 (m, 2H), 1.98 (t, J=10.7 Hz, 2H), 1.77-1.71 (m, 2H), 1.71-1.67 (m, 1H), 1.57-1.51 (m, 1H), 1.45-1.36 (m, 2H), 0.94 (d, J=6.3 Hz, 6H).

### Example-138 (VP-8-135A-E1, SPV-514)

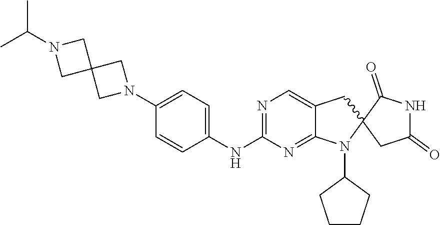 [SMILES: CC(C)N1CC2(CN(c3ccc(Nc4ncc5c(n4)N(C4CCCC4)C4(CC(=O)NC4=O)C5)cc3)C2)C1]

### Synthesis of 7′-cyclopentyl-2′-((4-(6-isopropyl-2,6-diazaspiro[3.3]heptan-2-yl)phenyl)amino)-5′,7′-dihydrospiro[pyrrolidine-3,6′-pyrrolo[2,3-d]pyrimidin]-2-one

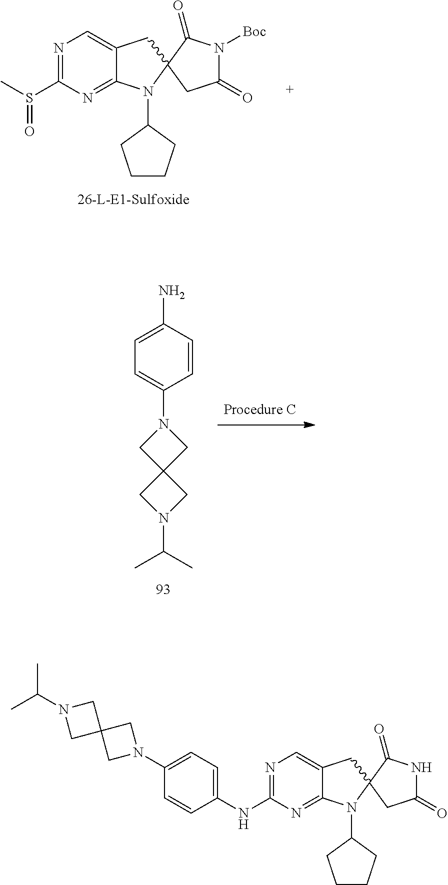 [SMILES: CS(=O)c1ncc2c(n1)N(C1CCCC1)C1(CC(=O)N(C(=O)OC(C)(C)C)C1=O)C2 ; CC(C)N1CC2(CN(c3ccc(N)cc3)C2)C1 ; CC(C)N1CC2(CN(c3ccc(Nc4ncc5c(n4)N(C4CCCC4)C4(CC(=O)NC4=O)C5)cc3)C2)C1]

was synthesized following the general procedure C, 8 mg (16% yield). MS(ESI) m/z 502.2 (M+H)⁺.

### Example-139 (VP-8-12A-E2, SPV-500)

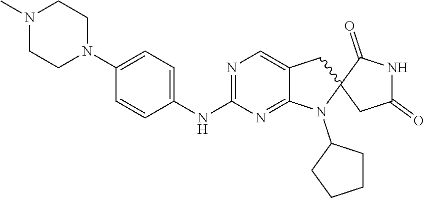 [SMILES: CN1CCN(c2ccc(Nc3ncc4c(n3)N(C3CCCC3)C3(CC(=O)NC3=O)C4)cc2)CC1]

### Synthesis of 7′-cyclopentyl-2′-((4-(4-methylpiperazin-1-yl)phenyl)amino)-5′,7′-dihydrospiro[pyrrolidine-3,6′-pyrrolo[2,3-d]pyrimidine]-2,5-dione

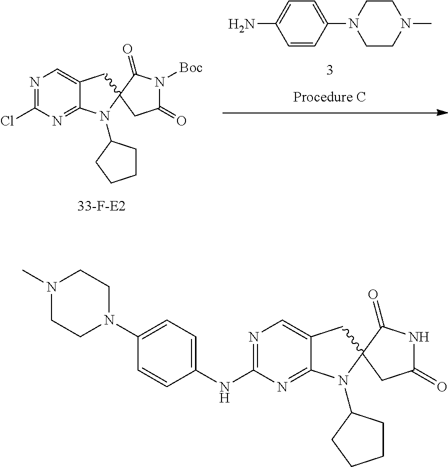 [SMILES: CN1CCN(c2ccc(N)cc2)CC1 ; CC(C)(C)OC(=O)N1C(=O)CC2(Cc3cnc(Cl)nc3N2C2CCCC2)C1=O ; CN1CCN(c2ccc(Nc3ncc4c(n3)N(C3CCCC3)C3(CC(=O)NC3=O)C4)cc2)CC1]

was synthesized following the general procedure C, 7 mg (15% yield). MS(ESI) m/z 462.3 (M+H)⁺.

### Example 4

### Inhibition Assays

Inhibition assays were performed using microfluidic LabChip 300 Drug Discovery System from Caliper Life Sciences. The assays were performed in six-point to 12-point concentration-response format (IC₅₀) (Nanosyn, Santa Clara, Calif.). The assay has a total volume of 10 μL consisting of 5 μL of enzyme and buffer and 5 μL of substrate and buffer. The test compounds were diluted in 100% dimethylsulfoxide (DMSO) using 3-fold dilution steps. The compounds were diluted in 100% dimethylsulfoxide (DMSO) and plated at 100-fold greater than the screening composition and serially diluted using 3-fold dilution steps. Compounds were tested in a single well for each dilution and the final concentration of DMSO was maintained at 1%.

The assay buffer contained 100 mM HEPES, pH 7.5, 0.01% Triton X-100, 0.1% bovine serum albumin (BSA), 5 mM MgCl₂; 1 mM dithiothreitol (DTT), 10 μM sodium orthovanadate, and 10 μM β-glycerophosphate.

The assay was conducted by adding 5 μL of enzyme, adenosine triphosphate (ATP) and buffer to an assay plate. A 100 nL aliquot of the diluted compounds were added to the plate. The assay was initiated by the addition of 5 μL of phosphoenolpyruvate substrate (PEP) in buffer. Specific concentrations for the enzyme, ATP, and PEP are shown in Table 1. The assays was incubated at 25° C. for 3 min for CDK2, CDK4 and CDK6 or 17 min for CDK9. The reaction was terminated by the addition of EDTA. The plates were analyzed using a Caliper LabChip 3000.

TABLE 1
Inhibition Assay Components and Concentrations

| | | ENZ Stock | ENZ Assay | ATP | PEP | Assay |
|---|---|---|---|---|---|---|
| Enzyme | Vendor | Conc (μM) | Conc (nM) | Conc (μM) | Conc (μM) | Time |
| CDK2-CYCLINA | Millipore | 1.8 | 0.1 | 50 | 1 | 3 |
| CDK2-CYCLINE | Millipore | 0.93 | 0.15 | 100 | 1 | 3 |
| CDK4-CYCLIND1 | Invitrogen | 3.5 | 1 | 200 | 1 | 3 |
| CDK6-CYCLIND3 | Carna | 3.13 | 2 | 300 | 1 | 3 |
| CDK9-CYCLINT1 | Carna | 2.86 | 5 | 10 | 1 | 17 |

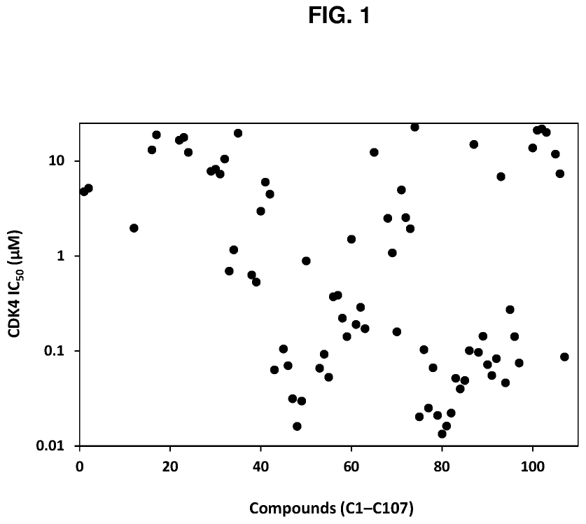

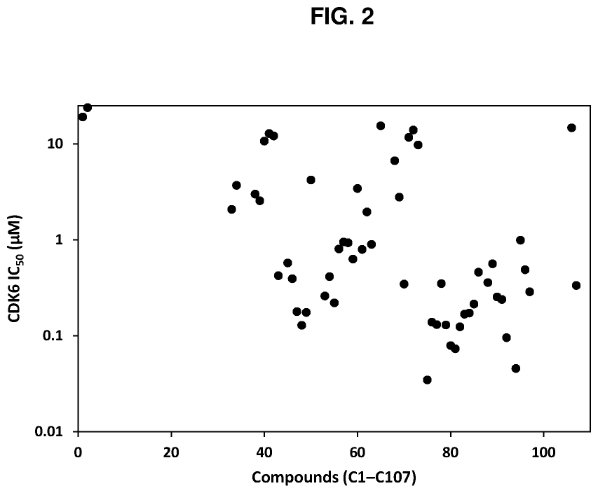

Results for controls and compounds described herein are shown in the table below. The results are graphically shown in FIG. 1 (CDK4) and FIG. 2 (CDK6).

TABLE 2
Inhibition Assay Results

| Compound | CDK4 IC50 (μM) | CDK6 IC50 (μM) |
|---|---|---|
| Staurosporine | 0.0535 | 0.0619 |
| Ribociclib | 0.00908 | 0.0243 |
| C1 | 4.74 | 19.1 |
| C2 | 5.16 | 23.8 |
| C3 | >30 | >30 |
| C4 | >30 | >30 |
| C5 | >30 | >30 |
| C6 | >30 | >30 |
| C7 | >30 | >30 |
| C8 | >30 | >30 |
| C9 | >30 | >30 |
| C10 | >30 | >30 |
| C11 | >30 | >30 |
| C12 | 1.96 | >30 |
| C13 | >30 | >30 |
| C14 | >30 | >30 |
| C15 | >30 | >30 |
| C16 | 13.1 | >30 |
| C17 | 18.8 | >30 |
| C18 | >30 | >30 |
| C19 | >30 | >30 |
| C20 | >30 | >30 |
| C21 | >30 | >30 |
| C22 | 16.5 | >30 |
| C23 | 17.7 | >30 |
| C24 | 12.3 | >30 |
| C25 | >30 | >30 |
| C26 | >30 | >30 |
| C27 | >30 | >30 |
| C28 | >30 | >30 |
| C29 | 7.78 | >30 |
| C30 | 8.21 | >30 |
| C31 | 7.28 | >30 |
| C32 | 10.5 | >30 |
| C33 | 0.694 | 2.08 |
| C34 | 1.16 | 3.7 |
| C35 | 19.6 | >30 |
| C36 | 29.3 | >30 |
| C37 | >30 | >30 |
| C38 | 0.631 | 2.99 |
| C39 | 0.53 | 2.55 |
| C40 | 2.96 | 10.7 |
| C41 | 5.98 | 12.8 |
| C42 | 4.46 | 12.1 |
| C43 | 0.0633 | 0.423 |
| C44 | >30 | >30 |
| C45 | 0.105 | 0.574 |
| C46 | 0.0701 | 0.394 |
| C47 | 0.0315 | 0.179 |
| C48 | 0.0161 | 0.129 |
| C49 | 0.0298 | 0.175 |
| C50 | 0.888 | 4.21 |
| C51 | >30 | >30 |
| C52 | >30 | >30 |
| C53 | 0.0658 | 0.26 |
| C54 | 0.0926 | 0.414 |
| C55 | 0.053 | 0.221 |
| C56 | 0.372 | 0.803 |
| C57 | 0.387 | 0.952 |
| C58 | 0.222 | 0.932 |
| C59 | 0.142 | 0.63 |
| C60 | 1.5 | 3.42 |
| C61 | 0.19 | 0.793 |
| C62 | 0.288 | 1.95 |
| C63 | 0.172 | 0.899 |
| C64 | >30 | >30 |
| C65 | 12.3 | 15.4 |
| C66 | 29.8 | >30 |
| C67 | 26.7 | >30 |
| C68 | 2.49 | 6.68 |
| C69 | 1.08 | 2.79 |
| C70 | 0.159 | 0.346 |
| C71 | 4.94 | 11.7 |
| C72 | 2.53 | 13.9 |
| C73 | 1.94 | 9.74 |
| C74 | 22.7 | 84.5 |
| C75 | 0.0203 | 0.0348 |
| C76 | 0.103 | 0.139 |
| C77 | 0.0251 | 0.131 |
| C78 | 0.0667 | 0.35 |
| C79 | 0.021 | 0.13 |
| C80 | 0.0134 | 0.0792 |
| C81 | 0.0162 | 0.0733 |
| C82 | 0.0222 | 0.124 |
| C83 | 0.0516 | 0.168 |
| C84 | 0.0399 | 0.173 |
| C85 | 0.0489 | 0.214 |
| C86 | 0.101 | 0.461 |
| C87 | 14.9 | 78.1 |
| C88 | 0.097 | 0.359 |
| C89 | 0.143 | 0.562 |
| C90 | 0.072 | 0.255 |
| C91 | 0.0554 | 0.239 |
| C92 | 0.0831 | 0.0955 |
| C93 | 6.84 | 31.8 |
| C94 | 0.0462 | 0.0456 |
| C95 | 0.273 | 0.993 |
| C96 | 0.142 | 0.488 |
| C97 | 0.0747 | 0.287 |
| C98 | 57.6 | 100 |
| C99 | 59.4 | 100 |
| C100 | 13.7 | 33.4 |
| C101 | 21 | 43.4 |
| C102 | 21.8 | 25.5 |
| C103 | 20 | 50.7 |
| C104 | 51 | 100 |
| C105 | 11.8 | 29.7 |
| C106 | 7.35 | 14.7 |
| C107 | 0.0866 | 0.335 |

### Example 5

Additional inhibition assays were performed as described above. The data is shown in Tables 3a and 3b.

TABLE 3a
Inhibition Assay Results

| | CDK1/ | CDK2 | CDK4 | CDK6 | CDK7 | CDK9 |
|---|---|---|---|---|---|---|
| | B | CyclinA | CyclinD1 | CyclinD3 | CyclinH | CyclinT1 |
| Com- | IC₅₀ | IC₅₀ | IC₅₀ | IC₅₀ | IC₅₀ | IC50 |
| pound | (μM) | (μM) | (μM) | (μM) | (μM) | (μM) |
| C197 | | 2.1 | 0.0517 | 0.258 | 22.1 | 0.0466 |
| | | | (0.028) | | | |
| C198 | | 0.247 | 0.0144 | 0.0706 | 0.168 | 0.0234 |
| C199 | | | 0.0332 | 0.103 | | |
| C200 | | | 0.0138 | 0.069 | | |
| | | | (0.0102) | | | |
| C201 | | | 0.0393 | 0.115 | | |
| C202 | | | 0.368 | 0.704 | | |
| C203 | | | 0.0245 | 0.104 | | |
| C204 | | | 2.03 | 11.8 | | |
| C205 | | | 0.0413 | 0.179 | | |
| C206 | | | 5.14 | 15.8 | | |
| C121 | >10 | >10 | 0.374 | | >10 | 0.132 |
| C108 | 6.35 | >10 | 0.124 | | >10 | 0.021 |
| C115 | 9.11 | >10 | 0.116 | | >10 | 0.149 |
| C118 | 3.82 | 4.9 | 0.0264 | | >10 | 0.016 |
| C123 | >10 | >10 | 0.955 | | >10 | 0.292 |
| C129 | 8.52 | >10 | 0.103 | | >10 | 0.0628 |
| C131 | >10 | >10 | 0.273 | | >10 | 0.298 |
| C132 | 6.93 | 9.66 | 0.0561 | | >10 | 0.0402 |
| C133 | >10 | >10 | 0.202 | | >10 | 0.125 |
| C134 | 1.78 | 2.28 | 0.0284 | | 8.1 | 0.0707 |
| C136 | 1.27 | 1.54 | 0.0257 | | 5.74 | 0.0805 |
| C137 | 1.65 | 3.33 | 0.202 | | 0.585 | 0.367 |
| C207 | >10 | 9.57 | 0.341 | | >10 | 0.148 |
| C208 | 2.02 | 2.28 | 0.0668 | | >10 | 0.0799 |
| C173 | 8.41 | >10 | 0.109 | | >10 | 0.0336 |
| C176 | 7.58 | >10 | 0.142 | | >10 | 0.027 |
| C178 | 1.05 | 0.681 | 0.0453 | | >10 | 0.00865 |
| C181 | 3.55 | 1.94 | 0.0679 | | >10 | 0.0602 |
| C149 | >10 | >10 | 0.593 | | >10 | 0.0852 |
| C209 | >10 | 5.01 | 1.38 | | >10 | 0.153 |
| C164 | >10 | >10 | 0.167 | | >10 | 0.117 |
| C165 | >10 | >10 | 0.702 | | >10 | 0.0914 |
| C168 | >10 | >10 | 1.31 | | >10 | 0.0231 |
| C170 | >10 | >10 | 0.233 | | >10 | 0.0282 |
| C150 | >10 | >10 | 0.522 | | >10 | 0.27 |
| C210 | >10 | >10 | 3.96 | | >10 | >10 |

TABLE 3b
Inhibition Assay Results

| | CDK1/ | CDK2 | CDK4 | CDK6 | CDK7 | CDK9 |
|---|---|---|---|---|---|---|
| | B | CyclinA | CyclinD1 | CyclinD3 | CyclinH | CyclinT1 |
| Com- | IC₅₀ | IC₅₀ | IC₅₀ | IC₅₀ | IC₅₀ | IC50 |
| pound | (μM) | (μM) | (μM) | (μM) | (μM) | (μM) |
| C203 | | | 0.0245 | 0.104 | | |
| C205 | | | 0.0413 | 0.179 | | |
| C204 | | | 2.03 | 11.8 | | |
| C206 | | | 5.14 | 15.8 | | |
| C121 | >10 | >10 | 0.374 | | >10 | 0.132 |
| C115 | 9.11 | >10 | 0.116 | | >10 | 0.149 |
| C118 | 3.82 | 4.9 | 0.0264 | | >10 | 0.016 |
| C123 | >10 | >10 | 0.955 | | >10 | 0.292 |
| C129 | 8.52 | >10 | 0.103 | | >10 | 0.0628 |
| C131 | >10 | >10 | 0.273 | | >10 | 0.298 |
| C132 | 6.93 | 9.66 | 0.0561 | | >10 | 0.0402 |
| C133 | >10 | >10 | 0.202 | | >10 | 0.125 |
| C207 | >10 | 9.57 | 0.341 | | >10 | 0.148 |
| C173 | 8.41 | >10 | 0.109 | | >10 | 0.0336 |
| C176 | 7.58 | >10 | 0.142 | | >10 | 0.027 |
| C178 | 1.05 | 0.681 | 0.0453 | | >10 | 0.00865 |
| C181 | 3.55 | 1.94 | 0.0679 | | >10 | 0.0602 |
| C149 | >10 | >10 | 0.593 | | >10 | 0.0852 |
| C209 | >10 | 5.01 | 1.38 | | >10 | 0.153 |
| C164 | >10 | >10 | 0.167 | | >10 | 0.117 |
| C165 | >10 | >10 | 0.702 | | >10 | 0.0914 |
| C168 | >10 | >10 | 1.31 | | >10 | 0.0231 |
| C170 | >10 | >10 | 0.233 | | >10 | 0.0282 |
| C210 | >10 | >10 | 3.96 | | >10 | >10 |
| C199 | | | 0.0332 | 0.103 | | |
| C200 | | | 0.0138 | 0.069 | | |
| | | | (0.0102) | | | |
| C201 | | | 0.0393 | 0.115 | | |
| C202 | | | 0.368 | 0.704 | | |
| C108 | 6.35 | >10 | 0.124 | | >10 | 0.021 |
| C134 | 1.78 | 2.28 | 0.0284 | | 8.1 | 0.0707 |
| C136 | 1.27 | 1.54 | 0.0257 | | 5.74 | 0.0805 |
| C208 | 2.02 | 2.28 | 0.0668 | | >10 | 0.0799 |
| C150 | >10 | >10 | 0.522 | | >10 | 0.27 |
| C137 | 1.65 | 3.33 | 0.202 | | 0.585 | 0.367 |
| C197 | | 2.1 | 0.0517 | 0.258 | 22.1 | 0.0466 |
| | | | (0.028) | | | |
| C198 | | 0.247 | 0.0144 | 0.0706 | 0.168 | 0.0234 |

### Example 6 (VP-8-69E1, compound 211)

Crystal Structure of VP-8-69E1, compound 211

A colorless block-like specimen of C20H₂₈N₄O₃S, approximate dimensions 0.083 mm×0.110 mm×0.129 mm, was used for the X-ray crystallographic analysis. The X-ray intensity data were measured (Δ=1.54178 Å).

The total exposure time was 2.28 hours. The frames were integrated with the Bruker SAINT software package using a narrow-frame algorithm. The integration of the data using a monoclinic unit cell yielded a total of 21553 reflections to a maximum θ angle of 74.43° (0.80 Å resolution), of which 4116 were independent (average redundancy 5.236, completeness=100.0%, Rᵢₙₜ=2.48%, R_{sig}=2.72%) and 4082 (99.17%) were greater than 2σ(F²). The final cell constants of a=9.0911(2) Å, b=10.0647(2) Å, c=11.5175(2) Å, β=97.5515(7)°, volume=1044.70(4) Å3, are based upon the refinement of the XYZ-centroids of 9826 reflections above 20 σ(I) with 7.743°<2θ<148.7°. Data were corrected for absorption effects using the Multi-Scan method (SADABS). The ratio of minimum to maximum apparent transmission was 0.946.

The calculated minimum and maximum transmission coefficients (based on crystal size) are 0.8200 and 0.8780.

The final anisotropic full-matrix least-squares refinement on F² with 257 variables converged at R1=2.23%, for the observed data and wR2=5.63% for all data. The goodness-of-fit was 1.043. The largest peak in the final difference electron density synthesis was 0.159 e−/Å³ and the largest hole was −0.205 e−/Å³ with an RMS deviation of 0.041 e−/Å³. On the basis of the final model, the calculated density was 1.286 g/cm³ and F(000), 432 e⁻.

TABLE 4
Crystal Data for VP-8-69E1

| Chemical formula | C₂₀H₂₈N₄O₄S | |
|---|---|---|
| Temperature | 100(2) K | |
| Formula weight | 404.52 g/mol | |
| Wavelength | 1.54178 Å | |
| Crystal size | 0.083 × 0.110 × 0.129 mm | |
| Crystal habit | Colorless block | |
| Crystal system | monoclinic | |
| Space group | P2₁ | |
| Unit cell dimensions | a = 9.0911(2) Å | α = 90° |
| | b = 10.0647(2) Å | β = 97.5515(7)° |
| | c = 11.5175(2) Å | γ = 90° |

| | |
|---|---|
| Volume | 1044.70(4) Å³ |
| Z | 2 |
| Density (calculated) | 1.286 g/cm³ |
| Absorption coefficient | 1.606 mm⁻¹ |
| F(000) | 423 |

TABLE 5
Data Collection and Structure Refinement for VP-8-69E1

| Theta range for data | 3.87 to 74.43° | |
|---|---|---|
| collection | | |
| Index ranges | −11 <= h <= 11, −12 <= | |
| | k <= 12, −14 <= l <= 14 | |
| Reflections collected | 21553 | |
| Independent reflections | 4116 [R(int) = 0.0248] | |
| Coverage of independent | 100.0% | |
| reflections | | |
| Absorption correction | Multi-Scan | |
| Max. and min. transmission | 0.8780 and 0.8200 | |
| Refinement method | Full-matrix least-squares on F² | |
| Refinement program | SHELXL-2018/3 (Sheldrick, 2018) | |
| Function minimized | Σ w(Fo² − Fc²)² | |
| Data/restraints/parameters | 4116/1/257 | |
| Goodness-of-fit on F² | 1.043 | |
| Final R indices | 4082 data; I > 2σ(I) | |
| Weighting scheme | w == 1/[σ²(Fo²) + (0.0285P)² + 0.1755P] | |
| | where P = (Fo² + 2Fc²)/3 | |
| Absolute structure parameter | 0.038(4) | R1 = 0.0223, |
| | | wR² = 0.0563 |
| Largest diff. peak and hole | 0.159 and −0.205 eÅ⁻³ | R1 = 0.0225, |
| | | wR² = 0.0563 |

| | |
|---|---|
| R.M.S. deviation from mean | 0.041 eÅ⁻³ |

The absolute stereochemistry of the structure (S at C1) was determined based on anomalous dispersion in the diffraction data, based on methods by Flack and Parsons.² The value of this parameter refined to approximately 4.9% (Flack X=0.049(13) using TWIN/BASF refinement), suggesting that a minor component of R enantiomer is present in the specimen. 2 Parson et al., Acta Cryst. B69: 249-259 (2013); Parsons et al., Acta Cryst. A68: 736-749 (2012)

TABLE 6
Atomic Coordinates and Equivalent Isotropic
Displacement Parameters (Å²) for VP-8-69E1.

| Atom | x/a | y/b | z/c | U(eq) |
|---|---|---|---|---|
| S1 | 0.51245(4) | 0.66883(4) | 0.45706(3) | 0.01743(10) |
| C1 | 0.02616(16) | 0.80653(16) | 0.17242(13) | 0.0119(3) |
| N1 | 0.20718(14) | 0.80461(13) | 0.04491(11) | 0.0126(3) |
| O2 | 0.26786(13) | 0.67052(13) | 0.89640(10) | 0.0199(2) |
| C2 | 0.87983(17) | 0.87001(18) | 0.11080(14) | 0.0165(3) |
| N2 | 0.98002(14) | 0.72206(13) | 0.26478(11) | 0.0124(3) |
| N3 | 0.76353(14) | 0.68890(13) | 0.35910(11) | 0.0122(3) |
| C3 | 0.76962(17) | 0.83355(15) | 0.19165(13) | 0.0123(3) |
| O1 | 0.05916(14) | 0.61617(12) | 0.04801(11) | 0.0199(3) |
| O3 | 0.37583(12) | 0.87232(11) | 0.93474(9) | 0.0141(2) |
| C5 | 0.62698(17) | 0.86923(17) | 0.19805(14) | 0.0144(3) |
| N4 | 0.54897(15) | 0.81918(14) | 0.28162(12) | 0.0150(3) |
| C4 | 0.83553(16) | 0.74327(15) | 0.27688(13) | 0.0106(3) |
| C8 | 0.22162(17) | 0.93445(15) | 0.10587(14) | 0.0136(3) |
| C6 | 0.62260(17) | 0.73258(16) | 0.35461(13) | 0.0124(3) |
| C7 | 0.09710(17) | 0.72566(16) | 0.08183(14) | 0.0130(3) |
| C10 | 0.28398(17) | 0.77252(16) | 0.95111(14) | 0.0137(3) |
| C9 | 0.14515(18) | 0.90900(16) | 0.21490(14) | 0.0147(3) |
| C15 | 0.10772(18) | 0.6602(2) | 0.46732(14) | 0.0249(4) |
| C14 | 0.1536(3) | 0.5237(3) | 0.5221(2) | 0.0396(6) |
| C13 | 0.1634(2) | 0.4288(2) | 0.4198(2) | 0.0325(5) |
| C12 | 0.0526(2) | 0.48584(18) | 0.32122(18) | 0.0251(4) |
| C11 | 0.08578(17) | 0.63447(16) | 0.33409(14) | 0.0151(3) |
| C20 | 0.6409(2) | 0.56741(18) | 0.55087(15) | 0.0201(3) |
| C19 | 0.56028(19) | 0.99311(17) | 0.85736(15) | 0.0192(3) |
| C18 | 0.37869(19) | 0.85919(18) | 0.72209(14) | 0.0196(3) |
| C17 | 0.57521(19) | 0.74376(18) | 0.86186(16) | 0.0209(3) |
| C16 | 0.47322(17) | 0.86346(17) | 0.84085(13) | 0.0142(3) |

U(eq) is defined as one third of the trace of the orthogonalized Uᵢⱼ tensor.

TABLE 7
Bond lengths (Å) for VP-8-69E1

| | | | |
|---|---|---|---|
| S1—C6 | 1.7657(16) | S1—C20 | 1.8002(18) |
| C1—N2 | 1.4655(19) | C1—C9 | 1.528(2) |
| C1—C7 | 1.531(2) | C1—C2 | 1.560(2) |
| N1—C7 | 1.388(2) | N1—C10 | 1.399(2) |
| N1—C8 | 1.4810(19) | O2—C10 | 1.203(2) |
| C2—C3 | 1.500(2) | C2—H2A | 0.99 |
| C2—H2B | 0.99 | N2—C4 | 1.356(2) |
| N2—C11 | 1.462(2) | N3—C4 | 1.337(2) |
| N3—C6 | 1.349(2) | C3—C5 | 1.357(2) |
| C3—C4 | 1.412(2) | O1—C7 | 1.204(2) |
| O3—C10 | 1.3353(19) | O3—C16 | 1.4876(17) |
| C5—N4 | 1.365(2) | C5—H5 | 0.95 |
| N4—C6 | 1.329(2) | C8—C9 | 1.535(2) |
| C8—H8A | 0.99 | C8—H8B | 0.99 |
| C9—H9A | 0.99 | C9—H9B | 0.99 |
| C15—C11 | 1.543(2) | C15—C14 | 1.546(3) |
| C15—H15A | 0.99 | C15—H15B | 0.99 |
| C14—C13 | 1.529(4) | C14—H14A | 0.99 |
| C14—H14B | 0.99 | C13—C12 | 1.527(3) |
| C13—H13A | 0.99 | C13—H13B | 0.99 |
| C12—C11 | 1.529(2) | C12—H12A | 0.99 |
| C12—H12B | 0.99 | C11—H11 | 1.0 |
| C20—H20A | 0.98 | C20—H20B | 0.98 |
| C20—H20C | 0.98 | C19—C16 | 1.525(2) |
| C19—H19A | 0.98 | C19—H19B | 0.98 |
| C19—H19C | 0.98 | C18—C16 | 1.517(2) |
| C18—H18A | 0.98 | C18—H18B | 0.98 |
| C18—H18C | 0.98 | C17—C16 | 1.520(2) |
| C17—H17A | 0.98 | C17—H17B | 0.98 |
| C17—H17C | 0.98 | | |

TABLE 8
Bond angles (°) for VP-8-69E1

| | | | |
|---|---|---|---|
| C6—S1—C20 | 103.13(8) | N2—C1—C9 | 114.67(12) |
| N2—C1—C7 | 111.88(12) | C9—C1—C7 | 103.48(12) |
| N2—C1—C2 | 105.05(12) | C9—C1—C2 | 113.26(13) |
| C7—C1—C2 | 108.55(12) | C7—N1—C10 | 123.89(14) |
| C7—N1—C8 | 112.49(12) | C10—N1—C8 | 123.20(13) |
| C3—C2—C1 | 102.50(12) | C3—C2—H2A | 111.3 |
| C1—C2—H2A | 111.3 | C3—C2—H2B | 111.3 |
| C1—C2—H2B | 111.3 | H2A—C2—H2B | 109.2 |
| C4—N2—C11 | 127.97(13) | C4—N2—C1 | 110.91(12) |
| C11—N2—C1 | 121.05(12) | C4—N3—C6 | 112.98(13) |
| C5—C3—C4 | 116.99(14) | C5—C3—C2 | 133.62(14) |
| C4—C3—C2 | 109.39(13) | C10—O3—C16 | 120.04(12) |
| C3—C5—N4 | 121.98(15) | C3—C5—H5 | 119.0 |
| N4—C5—H5 | 119.0 | C6—N4—C5 | 115.02(13) |
| N3—C4—N2 | 125.41(14) | N3—C4—C3 | 123.59(14) |
| N2—C4—C3 | 110.99(13) | N1—C8—C9 | 102.66(12) |
| N1—C8—H8A | 111.2 | C9—C8—H8A | 111.2 |
| N1—C8—H8B | 111.2 | C9—C8—H8B | 111.2 |
| H8A—C8—H8B | 109.1 | N4—C6—N3 | 129.40(14) |
| N4—C6—S1 | 112.23(11) | N3—C6—S1 | 118.37(12) |
| O1—C7—N1 | 127.76(15) | O1—C7—C1 | 125.35(15) |
| N1—C7—C1 | 106.84(13) | O2—C10—O3 | 127.10(14) |
| O2—C10—N1 | 124.23(15) | O3—C10—N1 | 108.67(13) |
| C1—C9—C8 | 103.21(12) | C1—C9—H9A | 111.1 |
| C8—C9—H9A | 111.1 | C1—C9—H9B | 111.1 |
| C8—C9—H9B | 111.1 | H9A—C9—H9B | 109.1 |
| C11—C15—C14 | 104.47(17) | C11—C15—H15A | 110.9 |
| C14—C15—H15A | 110.9 | C11—C15—H15B | 110.9 |
| C14—C15—H15B | 110.9 | H15A—C15—H15B | 108.9 |
| C13—C14—C15 | 106.28(16) | C13—C14—H14A | 110.5 |
| C15—C14—H14A | 110.5 | C13—C14—H14B | 110.5 |
| C15—C14—H14B | 110.5 | H14A—C14—H14B | 108.7 |
| C12—C13—C14 | 103.78(16) | C12—C13—H13A | 111.0 |
| C14—C13—H13A | 111.0 | C12—C13—H13B | 111.0 |
| C14—C13—H13B | 111.0 | H13A—C13—H13B | 109.0 |
| C13—C12—C11 | 101.15(15) | C13—C12—H12A | 111.5 |
| C11—C12—H12A | 111.5 | C13—C12—H12B | 111.5 |
| C11—C12—H12B | 111.5 | H12A—C12—H12B | 109.4 |

TABLE 9
Bond angles (°) for VP-8-69E1 (Cont'd).

| | | | |
|---|---|---|---|
| N2—C11—C12 | 115.45(14) | N2—C11—C15 | 115.58(14) |
| C12—C11—C15 | 104.97(15) | N2—C11—H11 | 106.7 |
| C12—C11—H11 | 106.7 | C15—C11—H11 | 106.7 |
| S1—C20—H20A | 109.5 | S1—C20—H20B | 109.5 |
| H20A—C20—H20B | 109.5 | S1—C20—H20C | 109.5 |
| H20A—C20—H20C | 109.5 | H20B—C20—H20C | 109.5 |
| C16—C19—H19A | 109.5 | C16—C19—H19B | 109.5 |
| H19A—C19—H19B | 109.5 | C16—C19—H19C | 109.5 |
| H19A—C19—H19C | 109.5 | H19B—C19—H19C | 109.5 |
| C16—C18—H18A | 109.5 | C16—C18—H18B | 109.5 |
| H18A—C18—H18B | 109.5 | C16—C18—H18C | 109.5 |
| H18A—C18—H18C | 109.5 | H18B—C18—H18C | 109.5 |
| C16—C17—H17A | 109.5 | C16—C17—H17B | 109.5 |
| H17A—C17—H17B | 109.5 | C16—C17—H17C | 109.5 |
| H17A—C17—H17C | 109.5 | H17B—C17—H17C | 109.5 |
| O3—C16—C18 | 109.65(12) | O3—C16—C17 | 109.91(13) |
| C18—C16—C17 | 112.48(14) | O3—C16—C19 | 101.93(12) |
| C18—C16—C19 | 111.09(14) | C17—C16—C19 | 111.26(13) |

TABLE 10
Torsion angles (°) for VP-8-69E1

| | | | |
|---|---|---|---|
| N2—C1—C2—C3 | 10.56(16) | C9—C1—C2—C3 | −115.30(14) |
| C7—C1—C2—C3 | 130.38(13) | C9—C1—N2—C4 | 115.59(14) |
| C7—C1—N2—C4 | −126.97(13) | C2—C1—N2—C4 | −9.40(16) |
| C9—C1—N2—C11 | −61.60(18) | C7—C1—N2—C11 | 55.84(18) |
| C2—C1—N2—C11 | 173.42(14) | C1—C2—C3—C5 | 171.11(17) |
| C1—C2—C3—C4 | −8.69(17) | C4—C3—C5—N4 | −1.0(2) |
| C2—C3—C5—N4 | 179.23(16) | C3—C5—N4—C6 | −0.7(2) |
| C6—N3—C4—N2 | 176.73(14) | C6—N3—C4—C3 | −2.0(2) |
| C11—N2—C4—N3 | 2.1(3) | C1—N2—C4—N3 | −174.84(14) |
| C11—N2—C4—C3 | −179.00(14) | C1—N2—C4—C3 | 4.06(17) |
| C5—C3—C4—N3 | 2.5(2) | C2—C3—C4—N3 | −177.67(14) |
| C5—C3—C4—N2 | −176.43(14) | C2—C3—C4—N2 | 3.41(18) |
| C7—N1—C8—C9 | 18.94(16) | C10—N1—C8—C9 | −168.17(14) |
| C5—N4—C6—N3 | 1.2(2) | C5—N4—C6—S1 | −178.21(11) |
| C4—N3—C6—N4 | 0.1(2) | C4—N3—C6—S1 | 179.52(11) |
| C20—S1—C6—N4 | −175.34(12) | C20—S1—C6—N3 | 5.19(14) |
| C10—N1—C7—O1 | 6.6(3) | C8—N1—C7—O1 | 179.46(15) |
| C10—N1—C7—C1 | −171.20(13) | C8—N1—C7—C1 | 1.63(16) |
| N2—C1—C7—O1 | 36.4(2) | C9—C1—C7—O1 | 160.40(15) |
| C2—C1—C7—O1 | −79.02(19) | N2—C1—C7—N1 | −145.67(13) |
| C9—C1—C7—N1 | −21.70(15) | C2—C1—C7—N1 | 98.88(15) |
| C16—O3—C10—O2 | −0.5(2) | C16—O3—C10—N1 | 179.17(12) |
| C7—N1—C10—O2 | −3.8(2) | C8—N1—C10—O2 | −175.83(15) |
| C7—N1—C10—O3 | 176.61(14) | C8—N1—C10—O3 | 4.53(19) |
| N2—C1—C9—C8 | 154.58(13) | C7—C1—C9—C8 | 32.45(15) |
| C2—C1—C9—C8 | −84.88(15) | N1—C8—C9—C1 | −31.21(15) |
| C11—C15—C14—C13 | 2.7(2) | C15—C14—C13—C12 | −28.3(2) |
| C14—C13—C12—C11 | 42.61(19) | C4—N2—C11—C12 | 66.7(2) |
| C1—N2—C11—C12 | −116.66(16) | C4—N2—C11—C15 | −56.4(2) |
| C1—N2—C11—C15 | 120.31(16) | C13—C12—C11—N2 | −169.76(14) |
| C13—C12—C11—C15 | −41.28(17) | C14—C15—C11—N2 | 152.37(15) |
| C14—C15—C11—C12 | 23.97(17) | C10—O3—C16—C18 | 64.35(18) |
| C10—O3—C16—C17 | −59.80(17) | C10—O3—C16—C19 | −177.88(13) |

TABLE 11
Anisotropic atomic displacement parameters (Å2) for VP-8-69E1.

| Atom | U₁₁ | U₂₂ | U₃₃ | U₂₃ | U₁₃ | U₁₂ |
|---|---|---|---|---|---|---|
| S1 | 0.01284(17) | 0.0242(2) | 0.01648(17) | 0.00303(16) | 0.00664(13) | −0.00036(16) |
| C1 | 0.0115(7) | 0.0120(7) | 0.0124(7) | 0.0008(6) | 0.0026(6) | −0.0005(6) |
| N1 | 0.0124(6) | 0.0113(6) | 0.0148(6) | −0.0018(5) | 0.0043(5) | −0.0019(5) |
| O2 | 0.0248(6) | 0.0157(5) | 0.0214(5) | −0.0057(5) | 0.0112(4) | −0.0046(5) |
| C2 | 0.0121(7) | 0.0204(8) | 0.0178(7) | 0.0070(7) | 0.0043(6) | 0.0036(6) |
| N2 | 0.0102(6) | 0.0143(6) | 0.0131(6) | 0.0038(5) | 0.0034(5) | 0.0018(5) |
| N3 | 0.0115(6) | 0.0133(7) | 0.0120(5) | −0.0004(5) | 0.0028(4) | −0.0002(5) |
| C3 | 0.0138(7) | 0.0110(7) | 0.0124(7) | 0.0003(6) | 0.0026(6) | 0.0004(5) |
| O1 | 0.0233(6) | 0.0161(5) | 0.0221(6) | −0.0064(5) | 0.0096(5) | −0.0086(5) |
| O3 | 0.0152(5) | 0.0143(5) | 0.0143(5) | −0.0006(4) | 0.0075(4) | −0.0020(4) |
| C5 | 0.0133(7) | 0.0150(7) | 0.0147(7) | 0.0014(6) | 0.0007(6) | 0.0028(6) |
| N4 | 0.0121(6) | 0.0170(7) | 0.0161(6) | 0.0002(5) | 0.0031(5) | 0.0016(5) |
| C4 | 0.0102(7) | 0.0104(7) | 0.0112(6) | −0.0023(6) | 0.0014(5) | −0.0004(5) |
| C8 | 0.0125(7) | 0.0110(7) | 0.0178(7) | −0.0017(6) | 0.0043(6) | −0.0020(6) |
| C6 | 0.0130(7) | 0.0131(7) | 0.0117(7) | −0.0020(6) | 0.0030(5) | −0.0013(6) |
| C7 | 0.0118(7) | 0.0147(7) | 0.0125(7) | 0.0010(6) | 0.0019(5) | −0.0008(6) |
| C10 | 0.0133(7) | 0.0138(7) | 0.0144(7) | 0.0015(6) | 0.0031(6) | 0.0002(6) |
| C9 | 0.0155(7) | 0.0141(7) | 0.0152(7) | −0.0026(6) | 0.0042(6) | −0.0019(6) |
| C15 | 0.0173(8) | 0.0418(11) | 0.0153(7) | 0.0041(8) | 0.0012(6) | 0.0087(8) |
| C14 | 0.0324(11) | 0.0564(15) | 0.0304(11) | 0.0261(10) | 0.0061(9) | 0.0084(10) |
| C13 | 0.0213(9) | 0.0276(10) | 0.0494(12) | 0.0231(9) | 0.0070(8) | 0.0053(8) |
| C12 | 0.0181(8) | 0.0172(8) | 0.0399(10) | 0.0076(8) | 0.0035(7) | 0.0020(7) |
| C11 | 0.0097(7) | 0.0188(8) | 0.0171(7) | 0.0048(6) | 0.0023(5) | 0.0036(5) |
| C20 | 0.0206(8) | 0.0247(9) | 0.0154(7) | 0.0046(7) | 0.0038(6) | −0.0013(7) |
| C19 | 0.0211(8) | 0.0200(8) | 0.0180(8) | 0.0015(7) | 0.0078(6) | −0.0056(7) |
| C18 | 0.0223(8) | 0.0233(8) | 0.0136(7) | 0.0012(7) | 0.0037(6) | −0.0017(7) |
| C17 | 0.0171(8) | 0.0210(8) | 0.0256(8) | 0.0016(7) | 0.0068(6) | 0.0043(7) |
| C16 | 0.0140(7) | 0.0172(7) | 0.0128(7) | 0.0014(6) | 0.0066(6) | −0.0003(6) |

TABLE 12
Hydrogen atomic coordinates and isotropic atomic
displacement parameters (Å2) for VP-8-69E1.

| Atom | x/a | y/b | z/c | U(eq) |
|---|---|---|---|---|
| H2A | −0.1106 | 0.9676 | 0.1042 | 0.02 |
| H2B | −0.1484 | 0.8320 | 0.0317 | 0.02 |
| H5 | −0.4200 | 0.9309 | 0.1427 | 0.017 |
| H8A | 0.1709 | 1.0057 | 0.0566 | 0.016 |
| H8B | 0.3271 | 0.9589 | 0.1276 | 0.016 |
| H9A | 0.2161 | 0.8732 | 0.2800 | 0.018 |
| H9B | 0.1002 | 0.9914 | 0.2412 | 0.018 |
| H15A | 0.0145 | 0.6918 | 0.4938 | 0.03 |
| H15B | 0.1864 | 0.7271 | 0.4887 | 0.03 |
| HUA | 0.2508 | 0.5302 | 0.5719 | 0.047 |
| HUB | 0.0789 | 0.4919 | 0.5709 | 0.047 |
| H13A | 0.2650 | 0.4275 | 0.3975 | 0.039 |
| H13B | 0.1355 | 0.3373 | 0.4398 | 0.039 |
| H12A | −0.0510 | 0.4656 | 0.3330 | 0.03 |
| H12B | 0.0712 | 0.4522 | 0.2437 | 0.03 |
| H11 | 0.1842 | 0.6493 | 0.3064 | 0.018 |
| H20A | −0.2847 | 0.6243 | 0.5956 | 0.03 |
| H20B | −0.3098 | 0.5052 | 0.5031 | 0.03 |
| H20C | −0.4130 | 0.5175 | 0.6050 | 0.03 |
| H19A | 0.4910 | 1.0681 | −0.1473 | 0.029 |
| H19B | 0.6246 | 1.0016 | −0.2042 | 0.029 |
| H19C | 0.6212 | 0.9929 | −0.0658 | 0.029 |
| H18A | 0.3202 | 0.7771 | −0.2847 | 0.029 |
| H18B | 0.4430 | 0.8618 | −0.3398 | 0.029 |
| H18C | 0.3118 | 0.9359 | −0.2859 | 0.029 |
| H17A | 0.6179 | 0.7409 | −0.0555 | 0.031 |
| H17B | 0.6551 | 0.7509 | −0.1872 | 0.031 |
| H17C | 0.5184 | 0.6623 | −0.1582 | 0.031 |

### Example 7 (VP-7-235E1, compound 212)

Crystal Structure of VP-7-235E1, compound 212

A colorless block-like specimen of C21H₃₀N₄O₃S, approximate dimensions 0.096 mm×0.113 mm×0.197 mm, was used for the X-ray crystallographic analysis. The X-ray intensity data were measured on a Bruker D8 VENTURE κ-geometry diffractometer system equipped with a Incoatec IμS 3.0 microfocus sealed tube (Cu Kα, λ=1.54178 Å) and a multilayer mirror monochromator.

The total exposure time was 3.12 hours. The frames were integrated with the Bruker SAINT software package using a narrow-frame algorithm. The integration of the data using a monoclinic unit cell yielded a total of 19791 reflections to a maximum θ angle of 77.41° (0.79 Å resolution), of which 4459 were independent (average redundancy 4.438, completeness=99.8%, Rᵢₙₜ=2.52%, R_{sig}=2.72%) and 4404 (98.77%) were greater than 2σ(F²). The final cell constants of a=9.3160(4) Å, b=10.3119(5) Å, c=11.3154(5) Å, β=97.0712(14°), volume=1078.75(8) Å³, are based upon the refinement of the XYZ-centroids of 659 reflections above 20 σ(I) with 9.637°<2θ<107.4°. Data were corrected for absorption effects using the Multi-Scan method (SADABS). The ratio of minimum to maximum apparent transmission was 0.912. The calculated minimum and maximum transmission coefficients (based on crystal size) are 0.7470 and 0.8640.

The structure was solved and refined using the Bruker SHELXTL Software Package, using the space group P₂₁, with Z=2 for the formula unit, C21H₃₀N₄O₃S. The final anisotropic full-matrix least-squares refinement on F² with 267 variables converged at R1=2.36%, for the observed data and wR2=6.16% for all data. The goodness-of-fit was 1.027. The largest peak in the final difference electron density synthesis was 0.357 e−/Å³ and the largest hole was −0.196 e−/Å³ with an RMS deviation of 0.038 e−/Å³. On the basis of the final model, the calculated density was 1.289 g/cm³ and F(000), 448 e⁻.

TABLE 13
Crystal Data for VP-7-235E1

| Chemical formula | C₂₁H₃₀N₄O₃S | |
|---|---|---|
| Temperature | 100(2) K | |
| Formula weight | 418.55 g/mol | |
| Wavelength | 1.54178 Å | |
| Crystal size | 0.096 × 0.113 × 0.197 mm | |
| Crystal habit | Colorless block | |
| Crystal system | monoclinic | |
| Space group | P2₁ | |
| Unit cell dimensions | a = 9.3160(4) Å | α = 90° |
| | b = 10.3119(5) Å | β = 97.0712(14)° |
| | c = 11.3154(5) Å | γ = 90° |

| | |
|---|---|
| Volume | 1078.75(8) Å³ |
| Z | 2 |
| Density (calculated) | 1.289 g/cm³ |
| Absorption coefficient | 1.572 mm⁻¹ |
| F(000) | 448 |

TABLE 14
Data Collection and Structure Refinement for VP-7-235E1

| Diffractometer | Bruker D8 VENTURE κ-geometry | |
|---|---|---|
| | diffractometer | |
| Radiation source | Incoatec IμS 3.0 microfocus sealed tube | |
| | (Cu Kα, λ = 1.54178 Å) | |
| Theta range for data | 3.94 to 77.41° | |
| collection | | |
| Index ranges | −11 <= h <= 10, −13 <= | |
| | k <= 13, −14 <= l <= 14 | |
| Reflections collected | 19791 | |
| Independent reflections | 4459 [R(int) = 0.0252] | |
| Coverage of independent | 99.8% | |
| reflections | | |
| Absorption correction | Multi-Scan | |
| Max. and min. transmission | 0.8640 and 0.7470 | |
| Structure solution technique | Direct methods | |
| Structure solution program | XT, VERSION 2014/5 (Sheldrick, 2015) | |
| Refinement method | Full-matrix least-squares on F² | |
| Refinement program | SHELXL-2018/3 (Sheldrick, 2018) | |
| Function minimized | Σ w(Fo² − Fc²)² | |
| Data/restraints/parameters | 4459/1/267 | |
| Goodness-of-fit on F² | 1.027 | |
| Final R indices | 4404 data; I > 2σ(I) | |
| Weighting scheme | w = 1/[σ²(Fo²) + (0.0333P)² + 0.1933P] | |
| | where P = (Fo² + 2Fc²)/3 | |
| Absolute structure parameter | 0.053(13) | R1 = 0.0236, |
| | | wR² = 0.0615 |
| Largest diff. peak and hole | 0.357 and −0.196 eÅ⁻³ | R1 = 0.0239, |
| | | wR² = 0.0616 |

| | |
|---|---|
| R.M.S. deviation from mean | 0.038 eÅ⁻³ |

The absolute stereochemistry of the structure (S at C1) was determined based on anomalous dispersion in the diffraction data using methods by Flack and Parsons.2 The value of this parameter refined to approximately 5.3% (Flack X 0.053(13) using TWIN/BASF refinement), suggesting that a minor component of R enantiomer is present in the specimen. 2 Parson et al., Acta Cryst. B69: 249-259 (2013); Parsons et al., Acta Cryst. A68: 736-749 (2012)

TABLE 15
Atomic Coordinates and Equivalent Isotropic
Displacement Parameters (Å²) for VP-7-235E1.

| Atom | x/a | y/b | z/c | U(eq) |
|---|---|---|---|---|
| S1 | 0.99691(4) | 0.11634(4) | 0.05915(4) | 0.01948(11) |
| O1 | 0.48394(12) | 0.11171(13) | 0.49849(10) | 0.0181(2) |
| O2 | 0.23114(13) | 0.13354(12) | 0.60888(11) | 0.0187(3) |
| O3 | 0.11608(12) | 0.32377(12) | 0.55671(10) | 0.0155(2) |
| N1 | 0.29543(14) | 0.26058(14) | 0.45706(12) | 0.0130(3) |
| N2 | 0.53661(14) | 0.16090(13) | 0.25464(12) | 0.0131(3) |
| N3 | 0.75101(14) | 0.13118(14) | 0.16075(12) | 0.0129(3) |
| N4 | 0.93575(15) | 0.29414(15) | 0.20550(13) | 0.0171(3) |
| C1 | 0.47574(16) | 0.25843(16) | 0.32842(14) | 0.0127(3) |
| C2 | 0.60397(17) | 0.35267(17) | 0.36892(16) | 0.0171(3) |
| C3 | 0.71708(17) | 0.30982(17) | 0.29351(14) | 0.0136(3) |
| C4 | 0.67142(16) | 0.19525(16) | 0.23224(13) | 0.0115(3) |
| C5 | 0.85082(18) | 0.35550(17) | 0.27794(15) | 0.0163(3) |
| C6 | 0.87943(16) | 0.18845(17) | 0.15199(14) | 0.0136(3) |
| C7 | 0.42215(16) | 0.19639(16) | 0.43861(14) | 0.0127(3) |
| C8 | 0.26166(17) | 0.36850(17) | 0.37216(15) | 0.0145(3) |
| C9 | 0.34110(17) | 0.32808(17) | 0.26756(15) | 0.0156(3) |
| C10 | 0.21391(17) | 0.22994(16) | 0.54935(15) | 0.0133(3) |
| C11 | 0.45740(16) | 0.04217(16) | 0.21588(14) | 0.0129(3) |
| C12 | 0.53867(18) | 0.92123(17) | 0.26656(16) | 0.0177(3) |
| C13 | 0.4557(2) | 0.79744(17) | 0.22735(18) | 0.0218(4) |
| C14 | 0.4227(2) | 0.79147(19) | 0.09208(18) | 0.0257(4) |
| C15 | 0.3376(2) | 0.91029(19) | 0.04401(17) | 0.0231(4) |
| C16 | 0.42036(19) | 0.03536(17) | 0.08034(15) | 0.0175(3) |
| C17 | 0.00191(17) | 0.30713(17) | 0.63707(15) | 0.0165(3) |
| C18 | 0.90669(18) | 0.19280(18) | 0.59289(17) | 0.0206(4) |
| C19 | 0.92061(19) | 0.43525(18) | 0.62010(17) | 0.0203(4) |
| C20 | 0.0691(2) | 0.2921(2) | 0.76588(17) | 0.0242(4) |
| C21 | 0.8814(2) | 0.0051(2) | 0.96945(17) | 0.0229(4) |

U(eq) is defined as one third of the trace of the orthogonalized Uᵢⱼ tensor.

TABLE 16
Bond lengths (Å) for VP-7-235E1

| | | | |
|---|---|---|---|
| S1—C6 | 1.7712(16) | S1—C21 | 1.799(2) |
| O1—C7 | 1.207(2) | O2—C10 | 1.200(2) |
| O3—C10 | 1.3388(19) | O3—C17 | 1.4918(18) |
| N1—C7 | 1.391(2) | N1—C10 | 1.401(2) |
| N1—C8 | 1.479(2) | N2—C4 | 1.358(2) |
| N2—C1 | 1.465(2) | N2—C11 | 1.468(2) |
| N3—C4 | 1.337(2) | N3—C6 | 1.349(2) |
| N4—C6 | 1.324(2) | N4—C5 | 1.363(2) |
| C1—C9 | 1.533(2) | C1—C7 | 1.538(2) |
| C1—C2 | 1.564(2) | C2—C3 | 1.502(2) |
| C2—H2A | 0.99 | C2—H2B | 0.99 |
| C3—C5 | 1.364(2) | C3—C4 | 1.409(2) |
| C5—H5 | 0.95 | C8—C9 | 1.529(2) |
| C8—H8A | 0.99 | C8—H8B | 0.99 |
| C9—H9A | 0.99 | C9—H9B | 0.99 |
| C11—C16 | 1.532(2) | C11—C12 | 1.533(2) |
| C11—H11 | 1.0 | C12—C13 | 1.530(2) |
| C12—H12A | 0.99 | C12—H12B | 0.99 |
| C13—C14 | 1.525(3) | C13—H13A | 0.99 |
| C13—H13B | 0.99 | C14—C15 | 1.523(3) |
| C14—H14A | 0.99 | C14—H14B | 0.99 |
| C15—C16 | 1.533(2) | C15—H15A | 0.99 |
| C15—H15B | 0.99 | C16—H16A | 0.99 |
| C16—H16B | 0.99 | C17—C20 | 1.522(3) |
| C17—C18 | 1.522(2) | C17—C19 | 1.523(2) |
| C18—H18A | 0.98 | C18—H18B | 0.98 |
| C18—H18C | 0.98 | C19—H19A | 0.98 |
| C19—H19B | 0.98 | C19—H19C | 0.98 |
| C20—H20A | 0.98 | C20—H20B | 0.98 |
| C20—H20C | 0.98 | C21—H21A | 0.98 |
| C21—H21B | 0.98 | C21—H21C | 0.98 |

TABLE 17
Bond angles (°) for VP-7-235E1

| | | | |
|---|---|---|---|
| C6—S1—C21 | 103.42(8) | C10—O3—C17 | 119.79(13) |
| C7—N1—C10 | 123.54(14) | C7—N1—C8 | 112.16(13) |
| C10—N1—C8 | 124.24(13) | C4—N2—C1 | 111.03(13) |
| C4—N2—C11 | 127.15(13) | C1—N2—C11 | 121.80(12) |
| C4—N3—C6 | 113.02(14) | C6—N4—C5 | 115.51(14) |
| N2—C1—C9 | 114.73(13) | N2—C1—C7 | 111.31(13) |
| C9—C1—C7 | 103.27(12) | N2—C1—C2 | 104.96(12) |
| C9—C1—C2 | 113.19(14) | C7—C1—C2 | 109.46(13) |
| C3—C2—C1 | 102.40(13) | C3—C2—H2A | 111.3 |
| C1—C2—H2A | 111.3 | C3—C2—H2B | 111.3 |
| C1—C2—H2B | 111.3 | H2A—C2—H2B | 109.2 |
| C5—C3—C4 | 116.84(14) | C5—C3—C2 | 133.47(16) |
| C4—C3—C2 | 109.63(13) | N3—C4—N2 | 125.32(15) |
| N3—C4—C3 | 123.76(14) | N2—C4—C3 | 110.91(14) |
| N4—C5—C3 | 121.64(16) | N4—C5—H5 | 119.2 |
| C3—C5—H5 | 119.2 | N4—C6—N3 | 129.21(15) |
| N4—C6—S1 | 112.21(11) | N3—C6—S1 | 118.57(13) |
| O1—C7—N1 | 127.93(15) | O1—C7—C1 | 125.16(14) |
| N1—C7—C1 | 106.85(13) | N1—C8—C9 | 102.31(13) |
| N1—C8—H8A | 111.3 | C9—C8—H8A | 111.3 |
| N1—C8—H8B | 111.3 | C9—C8—H8B | 111.3 |
| H8A—C8—H8B | 109.2 | C8—C9—C1 | 103.13(13) |
| C8—C9—H9A | 111.1 | C1—C9—H9A | 111.1 |
| C8—C9—H9B | 111.1 | C1—C9—H9B | 111.1 |
| H9A—C9—H9B | 109.1 | O2—C10—O3 | 127.48(15) |
| O2—C10—N1 | 123.90(15) | O3—C10—N1 | 108.61(14) |
| N2—C11—C16 | 112.35(13) | N2—C11—C12 | 111.08(13) |
| C16—C11—C12 | 112.17(14) | N2—C11—H11 | 107.0 |
| C16—C11—H11 | 107.0 | C12—C11—H11 | 107.0 |
| C13—C12—C11 | 111.21(14) | C13—C12—H12A | 109.4 |
| C11—C12—H12A | 109.4 | C13—C12—H12B | 109.4 |
| C11—C12—H12B | 109.4 | H12A—C12—H12B | 108.0 |
| C14—C13—C12 | 110.92(15) | C14—C13—H13A | 109.5 |
| C12—C13—H13A | 109.5 | C14—C13—H13B | 109.5 |
| C12—C13—H13B | 109.5 | H13A—C13—H13B | 108.0 |
| C15—C14—C13 | 110.97(15) | C15—C14—H14A | 109.4 |
| C13—C14—H14A | 109.4 | C15—C14—H14B | 109.4 |

TABLE 18
Bond angles (°) for VP-7-235E1 (Cont'd).

| | | | |
|---|---|---|---|
| C13—C14—H14B | 109.4 | H14A—C14—H14B | 108.0 |
| C14—C15—C16 | 111.01(15) | C14—C15—H15A | 109.4 |
| C16—C15—H15A | 109.4 | C14—C15—H15B | 109.4 |
| C16—C15—H15B | 109.4 | H15A—C15—H15B | 108.0 |
| C11—C16—C15 | 110.56(14) | C11—C16—H16A | 109.5 |
| C15—C16—H16A | 109.5 | C11—C16—H16B | 109.5 |
| C15—C16—H16B | 109.5 | H16A—C16—H16B | 108.1 |
| O3—C17—C20 | 110.82(13) | O3—C17—C18 | 108.62(13) |
| C20—C17—C18 | 112.95(15) | O3—C17—C19 | 101.78(13) |
| C20—C17—C19 | 110.40(15) | C18—C17—C19 | 111.70(14) |
| C17—C18—H18A | 109.5 | C17—C18—H18B | 109.5 |
| H18A—C18—H18B | 109.5 | C17—C18—H18C | 109.5 |
| H18A—C18—H18C | 109.5 | H18B—C18—H18C | 109.5 |
| C17—C19—H19A | 109.5 | C17—C19—H19B | 109.5 |
| H19A—C19—H19B | 109.5 | C17—C19—H19C | 109.5 |
| H19A—C19—H19C | 109.5 | H19B—C19—H19C | 109.5 |
| C17—C20—H20A | 109.5 | C17—C20—H20B | 109.5 |
| H20A—C20—H20B | 109.5 | C17—C20—H20C | 109.5 |
| H20A—C20—H20C | 109.5 | H20B—C20—H20C | 109.5 |
| S1—C21—H21A | 109.5 | S1—C21—H21B | 109.5 |
| H21A—C21—H21B | 109.5 | S1—C21—H21C | 109.5 |
| H21A—C21—H21C | 109.5 | H21B—C21—H21C | 109.5 |

TABLE 19
Torsion angles (°) for VP-7-234E1

| | | | |
|---|---|---|---|
| C4—N2—C1—C9 | 115.72(15) | C11—N2—C1—C9 | −65.73(19) |
| C4—N2—C1—C7 | −127.47(14) | C11—N2—C1—C7 | 51.08(19) |
| C4—N2—C1—C2 | −9.15(18) | C11—N2—C1—C2 | 169.40(14) |
| N2—C1—C2—C3 | 10.05(17) | C9—C1—C2—C3 | −115.78(15) |
| C7—C1—C2—C3 | 129.61(14) | C1—C2—C3—C5 | 174.98(18) |
| C1—C2—C3—C4 | −8.11(18) | C6—N3—C4—N2 | 178.33(15) |
| C6—N3—C4—C3 | −0.8(2) | C1—N2—C4—N3 | −175.00(15) |
| C11—N2—C4—N3 | 6.5(3) | C1—N2—C4—C3 | 4.20(18) |
| C11—N2—C4—C3 | −174.25(15) | C5—C3—C4—N3 | −0.4(2) |
| C2—C3—C4—N3 | −177.84(15) | C5—C3—C4—N2 | −179.57(15) |
| C2—C3—C4—N2 | 2.95(19) | C6—N4—C5—C3 | 0.1(2) |
| C4—C3—C5—N4 | 0.7(2) | C2—C3—C5—N4 | 177.47(18) |
| C5—N4—C6—N3 | −1.5(3) | C5—N4—C6—S1 | 179.58(12) |
| C4—N3—C6—N4 | 1.8(2) | C4—N3—C6—S1 | −179.32(11) |
| C21—S1—C6—N4 | −165.45(13) | C21—S1—C6—N3 | 15.50(15) |
| C10—N1—C7—O1 | −3.4(3) | C8—N1—C7—O1 | 173.68(17) |
| C10—N1—C7—C1 | 179.37(14) | C8—N1—C7—C1 | −3.60(17) |
| N2—C1—C7—O1 | 40.7(2) | C9—C1—C7—O1 | 164.31(16) |
| C2—C1—C7—O1 | −74.9(2) | N2—C1—C7—N1 | −141.92(13) |
| C9—C1—C7—N1 | −18.32(16) | C2—C1—C7—N1 | 102.51(15) |
| C7—N1—C8—C9 | 23.99(17) | C10—N1—C8—C9 | −159.00(14) |
| N1—C8—C9—C1 | −33.90(15) | N2—C1—C9—C8 | 153.46(13) |
| C7—C1—C9—C8 | 32.15(16) | C2—C1—C9—C8 | −86.11(16) |
| C17—O3—C10—O2 | −7.1(2) | C17—O3—C10—N1 | 171.94(13) |
| C7—N1—C10—O2 | −12.6(2) | C8—N1—C10—O2 | 170.70(16) |
| C7—N1—C10—O3 | 168.28(14) | C8—N1—C10—O3 | −8.4(2) |
| C4—N2—C11—C16 | −65.6(2) | C1—N2—C11—C16 | 116.14(16) |
| C4—N2—C11—C12 | 61.0(2) | C1—N2—C11—C12 | −117.30(15) |
| N2—C11—C12—C13 | 179.43(14) | C16—C11—C12—C13 | −53.90(18) |
| C11—C12—C13—C14 | 54.87(19) | C12—C13—C14—C15 | −57.1(2) |
| C13—C14—C15—C16 | 57.8(2) | N2—C11—C16—C15 | −179.86(13) |
| C12—C11—C16—C15 | 54.17(18) | C14—C15—C16—C11 | −55.91(19) |
| C10—O3—C17—C20 | 60.99(19) | C10—O3—C17—C18 | −63.65(18) |
| C10—O3—C17—C19 | 178.39(14) | | |

TABLE 20
Anisotropic atomic displacement parameters (Å2) for VP-7-235E1.

| Atom | U₁₁ | U₂₂ | U₃₃ | U₂₃ | U₁₃ | U₁₂ |
|---|---|---|---|---|---|---|
| S1 | 0.01462(18) | 0.0245(2) | 0.0211(2) | −0.00416(17) | 0.00933(14) | 0.00064(16) |
| O1 | 0.0189(5) | 0.0179(6) | 0.0182(5) | 0.0034(5) | 0.0047(4) | 0.0057(5) |
| O2 | 0.0185(5) | 0.0169(6) | 0.0217(6) | 0.0032(5) | 0.0072(4) | 0.0019(5) |
| O3 | 0.0143(5) | 0.0158(6) | 0.0181(6) | −0.0001(5) | 0.0085(4) | 0.0020(4) |
| N1 | 0.0117(6) | 0.0127(6) | 0.0152(6) | 0.0002(5) | 0.0042(5) | 0.0016(5) |
| N2 | 0.0109(6) | 0.0133(6) | 0.0160(6) | −0.0034(5) | 0.0058(5) | −0.0010(5) |
| N3 | 0.0119(6) | 0.0145(7) | 0.0130(6) | −0.0005(5) | 0.0039(4) | 0.0005(5) |
| N4 | 0.0138(6) | 0.0209(7) | 0.0174(7) | −0.0018(6) | 0.0051(5) | −0.0027(5) |
| C1 | 0.0124(7) | 0.0119(7) | 0.0142(7) | −0.0023(6) | 0.0035(6) | 0.0000(6) |
| C2 | 0.0137(7) | 0.0167(8) | 0.0220(8) | −0.0067(7) | 0.0071(6) | −0.0041(6) |
| C3 | 0.0131(7) | 0.0147(8) | 0.0133(7) | −0.0004(6) | 0.0030(5) | 0.0001(6) |
| C4 | 0.0103(7) | 0.0128(7) | 0.0113(7) | 0.0014(6) | 0.0012(5) | 0.0007(6) |
| C5 | 0.0140(7) | 0.0176(8) | 0.0177(8) | −0.0026(7) | 0.0036(6) | −0.0032(6) |
| C6 | 0.0123(7) | 0.0166(8) | 0.0124(7) | 0.0019(6) | 0.0036(5) | 0.0021(6) |
| C7 | 0.0107(7) | 0.0134(7) | 0.0143(7) | −0.0034(6) | 0.0026(5) | −0.0008(6) |
| C8 | 0.0125(7) | 0.0144(8) | 0.0171(8) | 0.0021(6) | 0.0041(6) | 0.0024(6) |
| C9 | 0.0141(7) | 0.0176(8) | 0.0157(8) | 0.0020(6) | 0.0043(6) | 0.0026(6) |
| C10 | 0.0112(7) | 0.0133(8) | 0.0156(8) | −0.0031(6) | 0.0021(6) | −0.0011(6) |
| C11 | 0.0116(7) | 0.0126(8) | 0.0152(8) | −0.0020(6) | 0.0039(6) | −0.0029(6) |
| C12 | 0.0174(8) | 0.0145(8) | 0.0212(8) | −0.0005(7) | 0.0018(6) | 0.0001(6) |
| C13 | 0.0215(8) | 0.0119(8) | 0.0325(10) | −0.0012(7) | 0.0051(7) | −0.0012(7) |
| C14 | 0.0257(9) | 0.0189(9) | 0.0328(10) | −0.0116(8) | 0.0049(7) | −0.0035(7) |
| C15 | 0.0237(8) | 0.0222(9) | 0.0226(9) | −0.0072(7) | −0.0005(7) | −0.0070(7) |
| C16 | 0.0191(8) | 0.0173(8) | 0.0159(8) | −0.0018(7) | 0.0020(6) | −0.0035(6) |
| C17 | 0.0143(7) | 0.0187(8) | 0.0182(8) | 0.0004(7) | 0.0092(6) | 0.0005(6) |
| C18 | 0.0151(7) | 0.0170(8) | 0.0306(9) | 0.0000(7) | 0.0069(7) | 0.0003(7) |
| C19 | 0.0197(8) | 0.0184(8) | 0.0250(9) | −0.0014(7) | 0.0114(7) | 0.0029(7) |
| C20 | 0.0231(8) | 0.0326(10) | 0.0185(8) | 0.0006(8) | 0.0084(7) | 0.0025(8) |
| C21 | 0.0242(9) | 0.0251(9) | 0.0204(9) | −0.0075(7) | 0.0063(7) | 0.0032(7) |

TABLE 21
Hydrogen atomic coordinates and isotropic atomic
displacement parameters (Å2) for VP-7-235E1.

| Atom | x/a | y/b | z/c | U(eq) |
|---|---|---|---|---|
| H2A | 0.5759 | 0.4442 | 0.3527 | 0.02 |
| H2B | 0.6381 | 0.3424 | 0.4548 | 0.02 |
| H5 | 0.8857 | 0.4320 | 0.3186 | 0.02 |
| H8A | 0.2987 | 0.4521 | 0.4066 | 0.017 |
| H8B | 0.1562 | 0.3759 | 0.3478 | 0.017 |
| H9A | 0.3680 | 0.4046 | 0.2223 | 0.019 |
| H9B | 0.2810 | 0.2691 | 0.2130 | 0.019 |
| H11 | 0.3636 | 0.0448 | 0.2503 | 0.016 |
| H12A | 0.6354 | −0.0815 | 0.2390 | 0.021 |
| H12B | 0.5524 | −0.0739 | 0.3546 | 0.021 |
| H13A | 0.5139 | −0.2791 | 0.2562 | 0.026 |
| H13B | 0.3640 | −0.2050 | 0.2632 | 0.026 |
| H14A | 0.5144 | −0.2134 | 0.0565 | 0.031 |
| H14B | 0.3661 | −0.2878 | 0.0690 | 0.031 |
| H15A | 0.2428 | −0.0882 | 0.0752 | 0.028 |
| H15B | 0.3195 | −0.0949 | −0.0439 | 0.028 |
| H16A | 0.5106 | 0.0382 | 0.0424 | 0.021 |
| H16B | 0.3606 | 0.1112 | 0.0522 | 0.021 |
| H18A | −0.1269 | 0.2040 | 0.5080 | 0.031 |
| H18B | −0.1768 | 0.1885 | 0.6376 | 0.031 |
| H18C | −0.0375 | 0.1123 | 0.6046 | 0.031 |
| H19A | −0.0128 | 0.5071 | 0.6430 | 0.03 |
| H19B | −0.1582 | 0.4363 | 0.6700 | 0.03 |
| H19C | −0.1194 | 0.4448 | 0.5363 | 0.03 |
| H20A | 0.1189 | 0.2084 | 0.7759 | 0.036 |
| H20B | −0.0070 | 0.2960 | 0.8184 | 0.036 |
| H20C | 0.1386 | 0.3623 | 0.7864 | 0.036 |
| H21A | 0.8236 | −0.0444 | 0.0205 | 0.034 |
| H21B | 0.9408 | −0.0546 | −0.0714 | 0.034 |
| H21C | 0.8169 | 0.0536 | −0.0897 | 0.034 |

What is claimed:

1. A compound or a pharmaceutically acceptable salt thereof comprising Formula I or II: 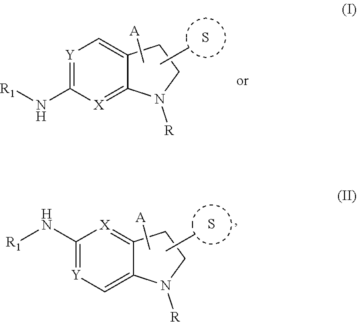
wherein
A is none, Me, CO, or gem-difluoro;
X and Y are independently, C, N, or C—F; [symbol]
is 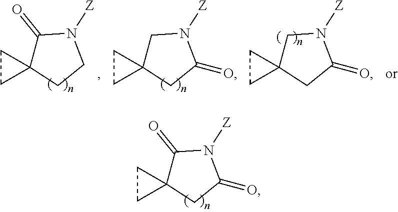
bonded in a spiro-manner, 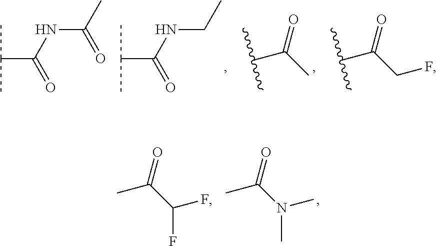
CN; where Z is H, Me, or CH₂CH₂OH, CH₂CH₂CN, CH₂CH₂F, COCH₃, COCH═CH; and n is 0, 1, 2, or 3;
R is —(CH₂)ₙ, 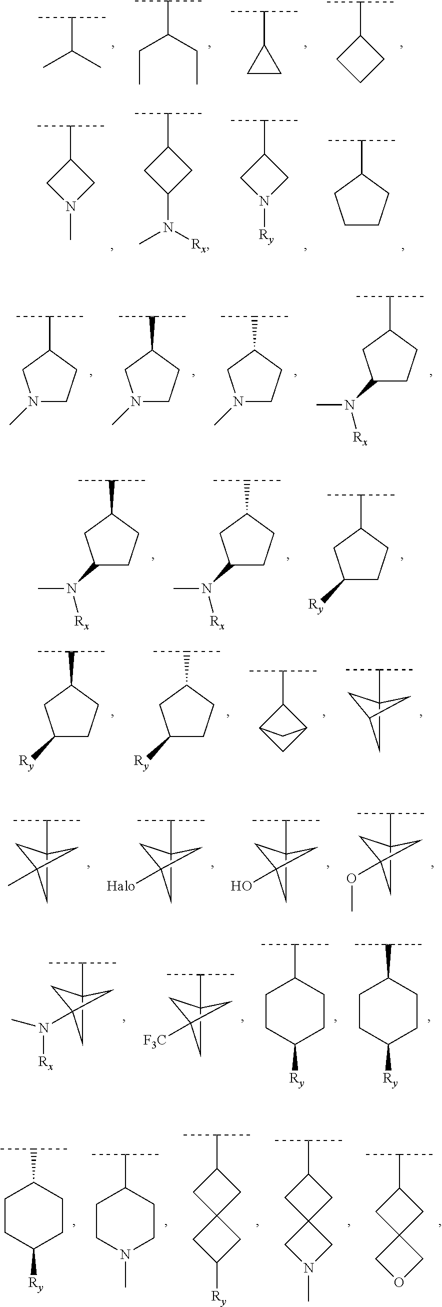 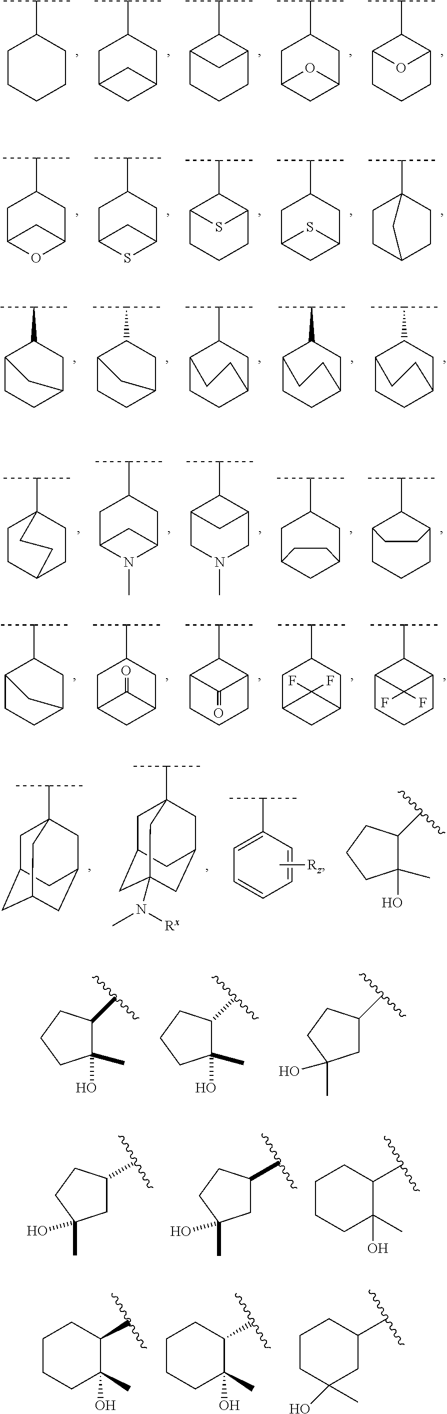 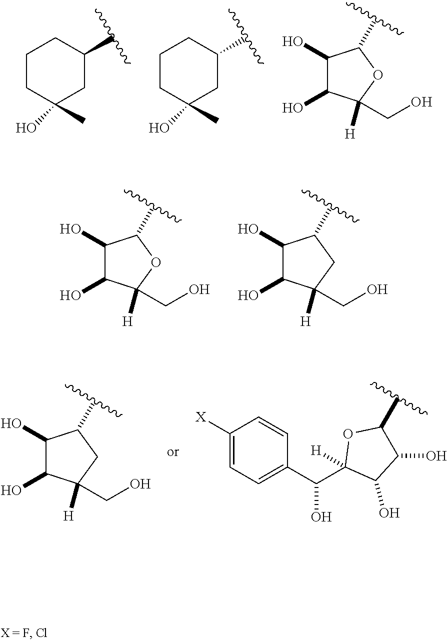
where Rₓ is H or Me, R_{y} is Me, OH, or OMe; R_{z} is H, Me, OMe, OH, N, F, or CF₃; and n is 1 to 11; and
R₁ is: 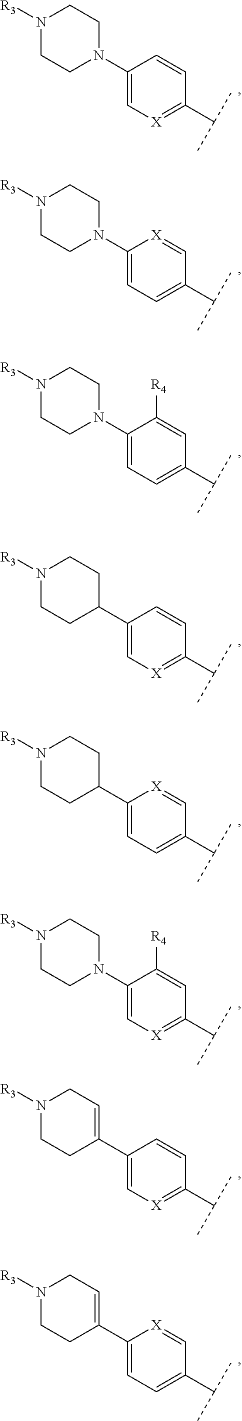 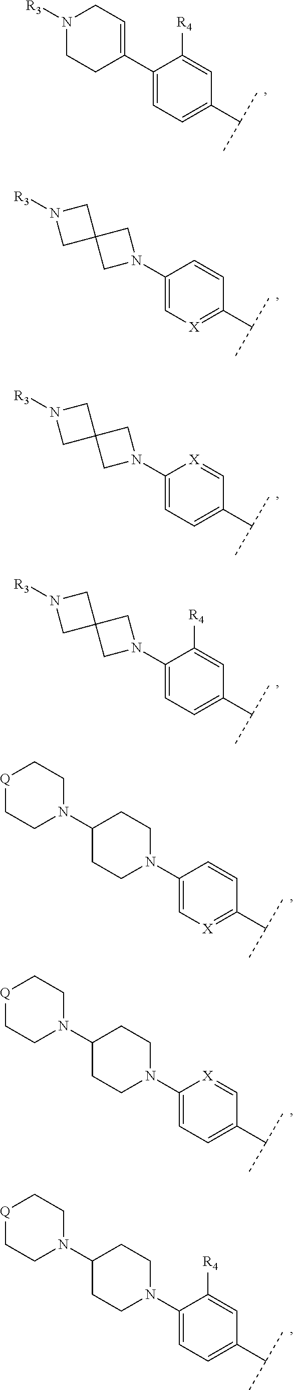 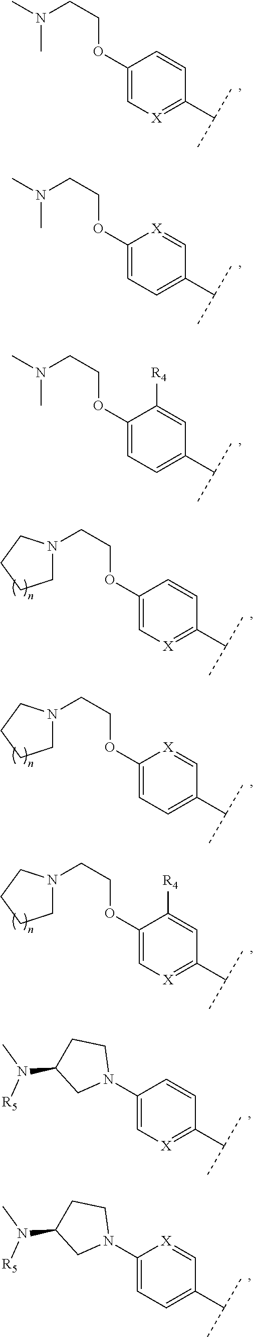 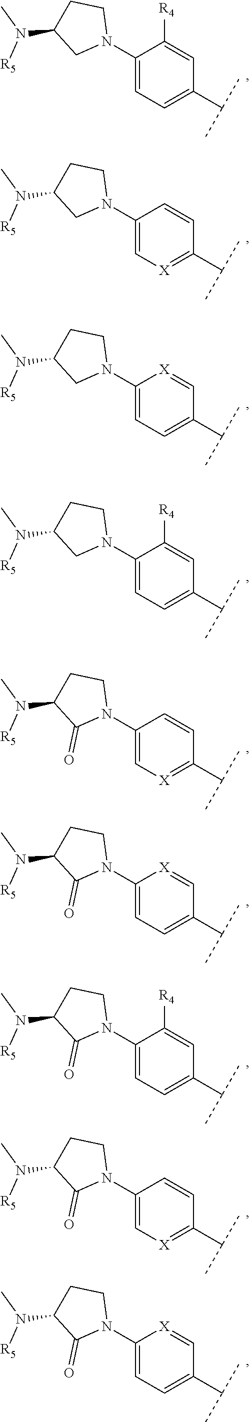 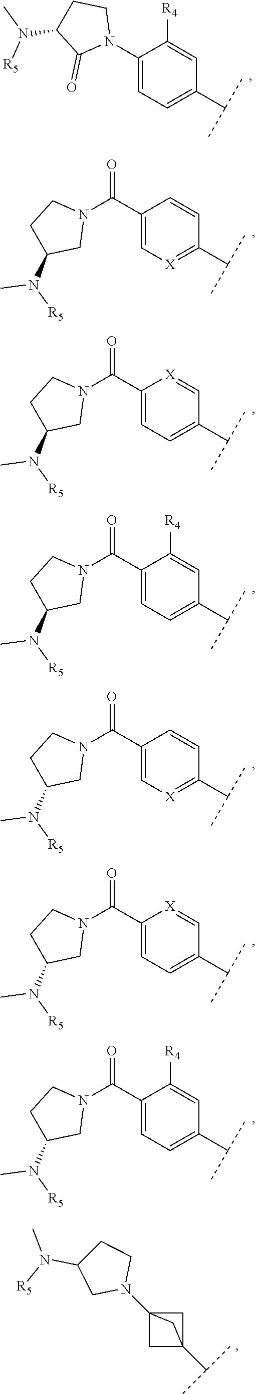 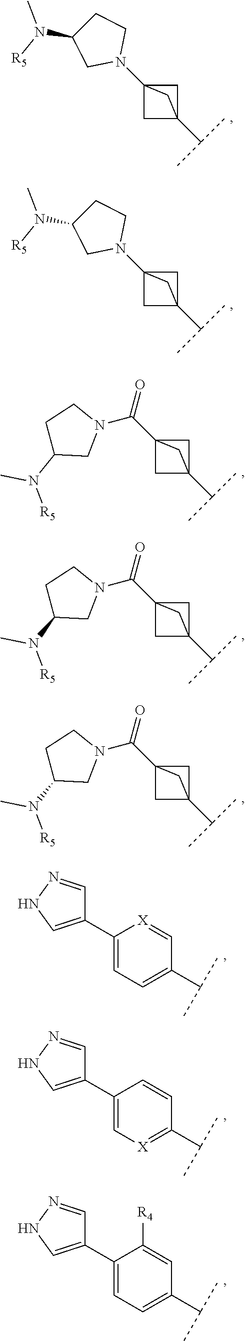 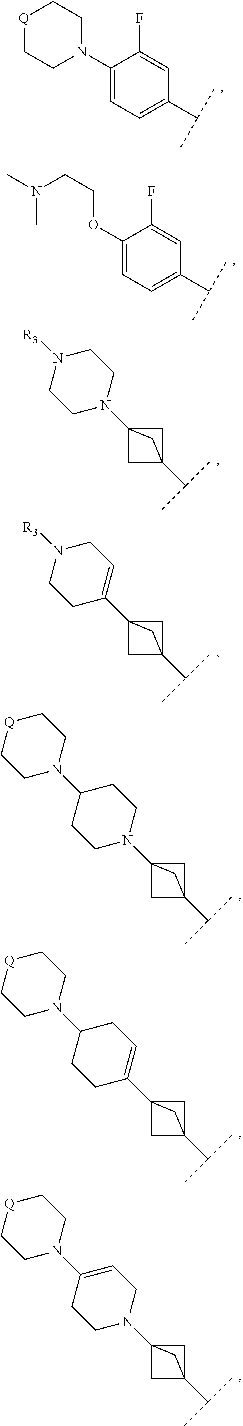 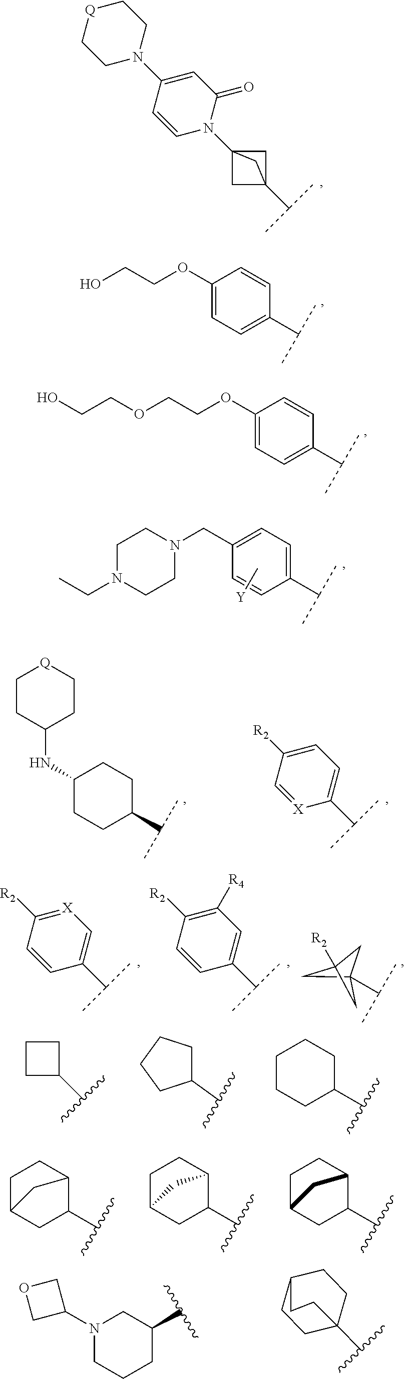 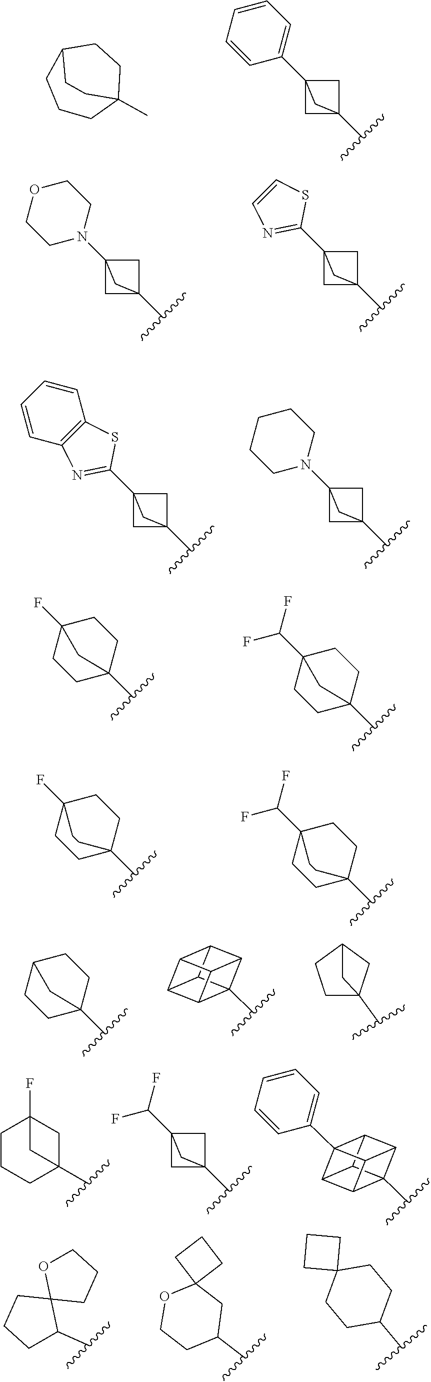 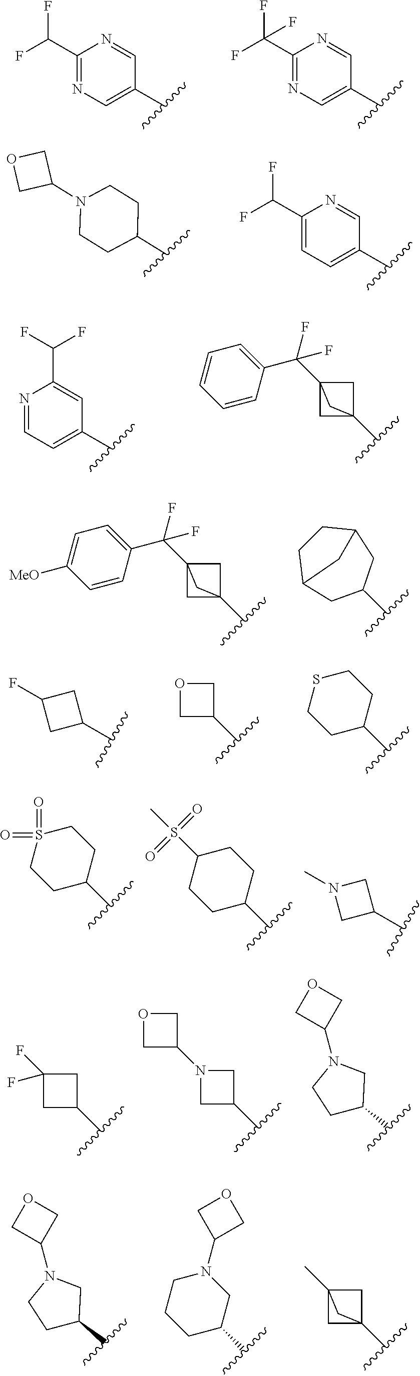 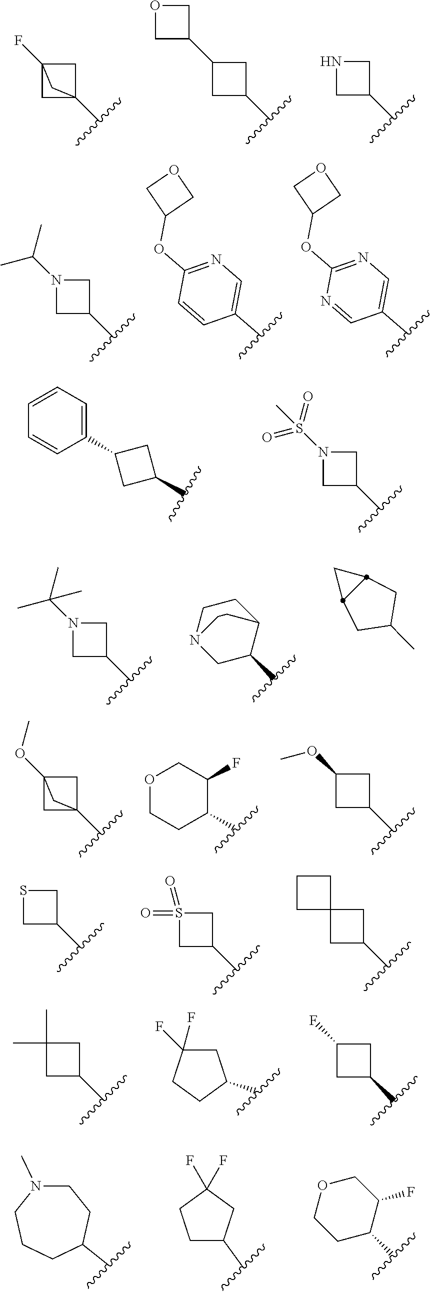 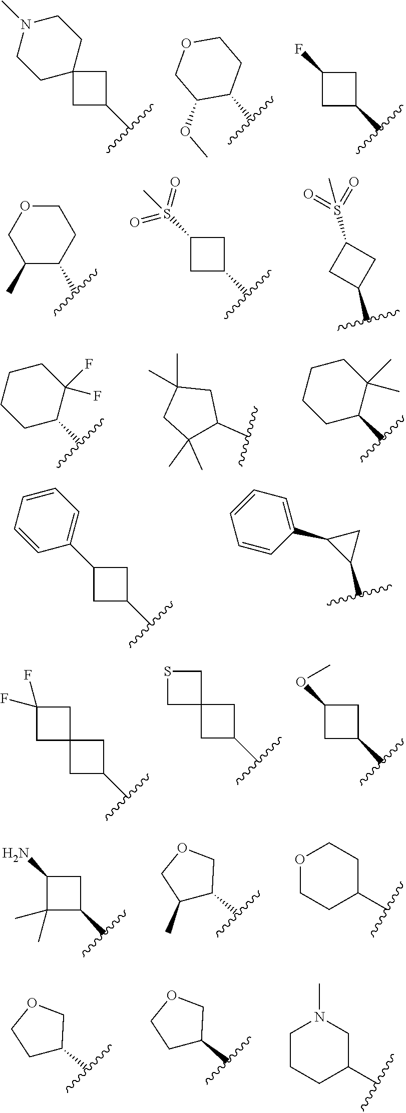 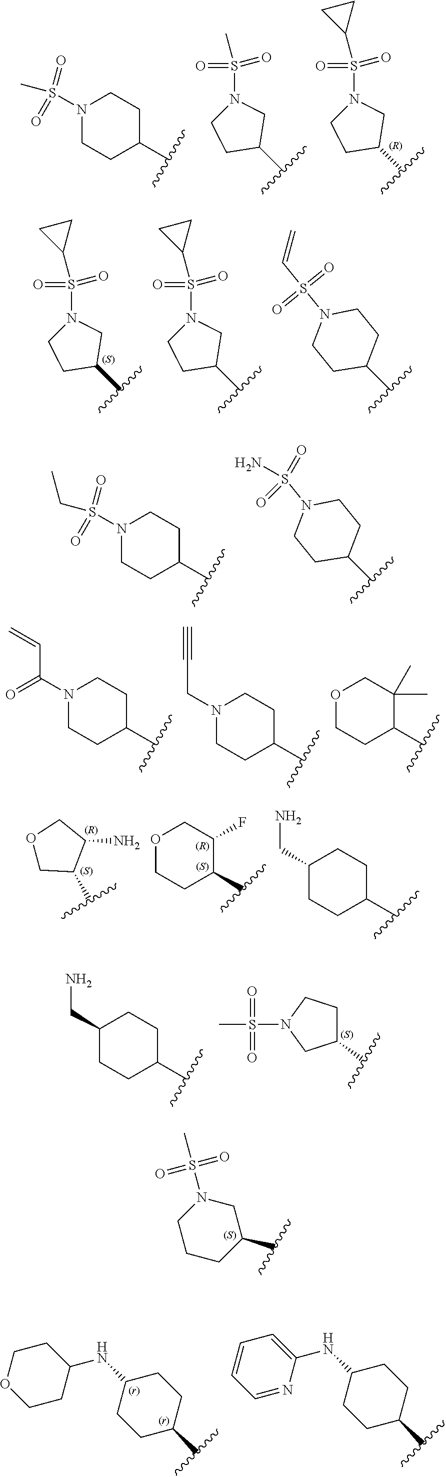 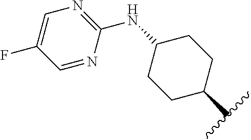 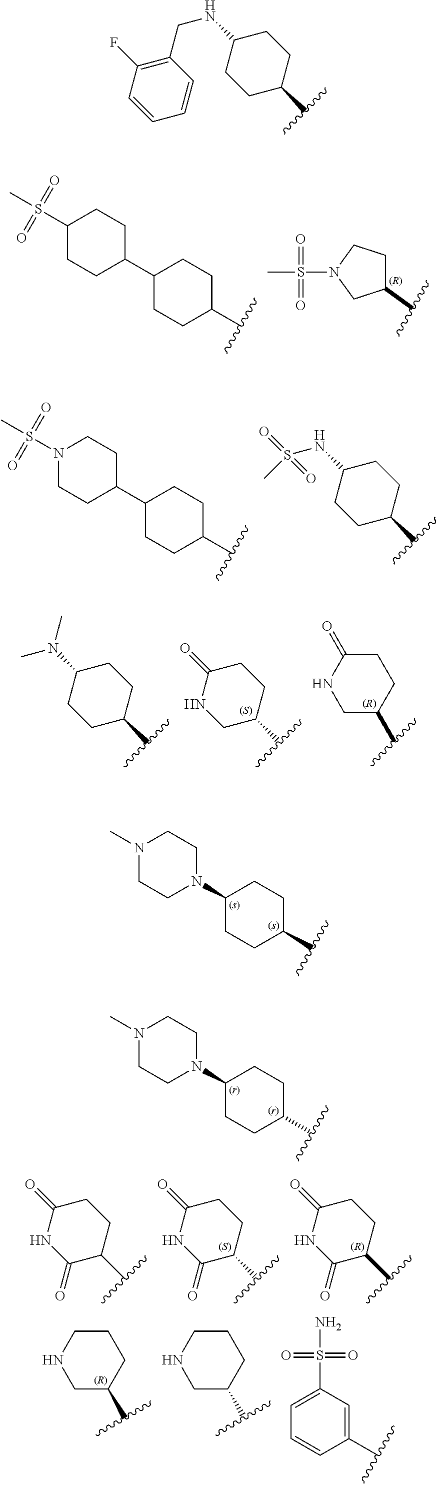 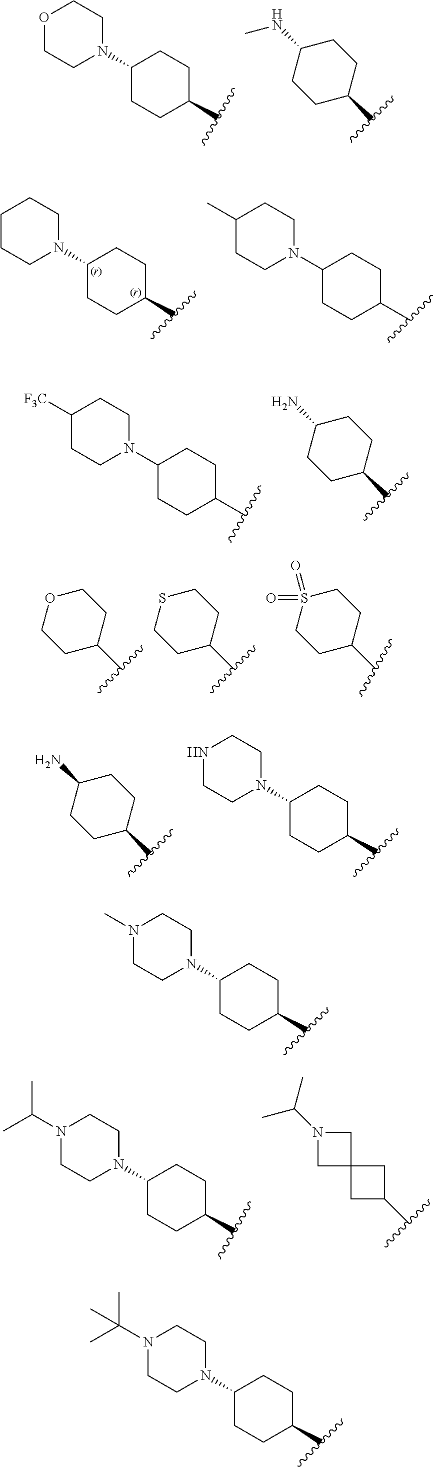 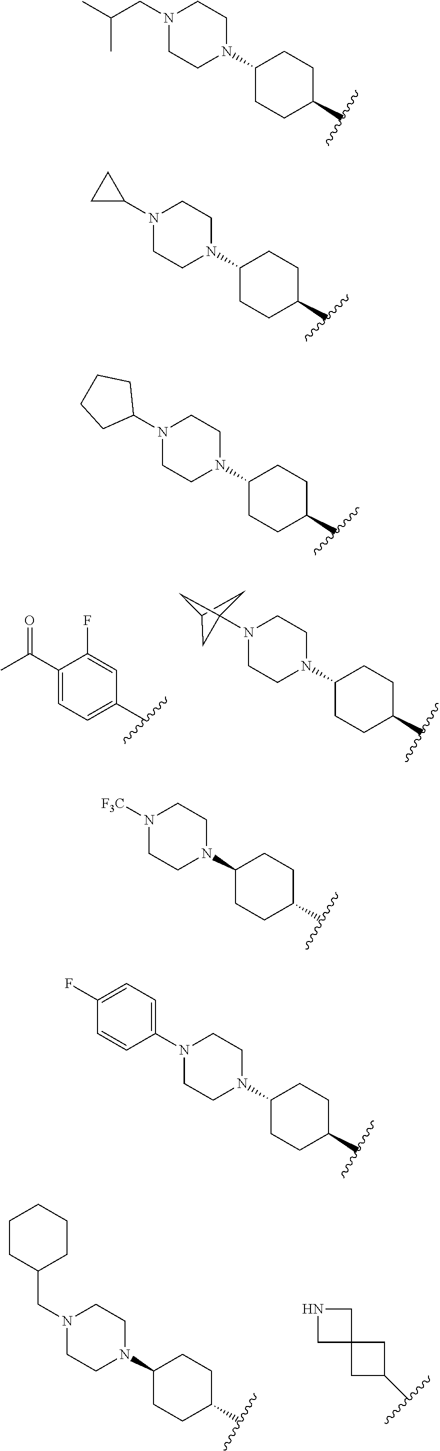 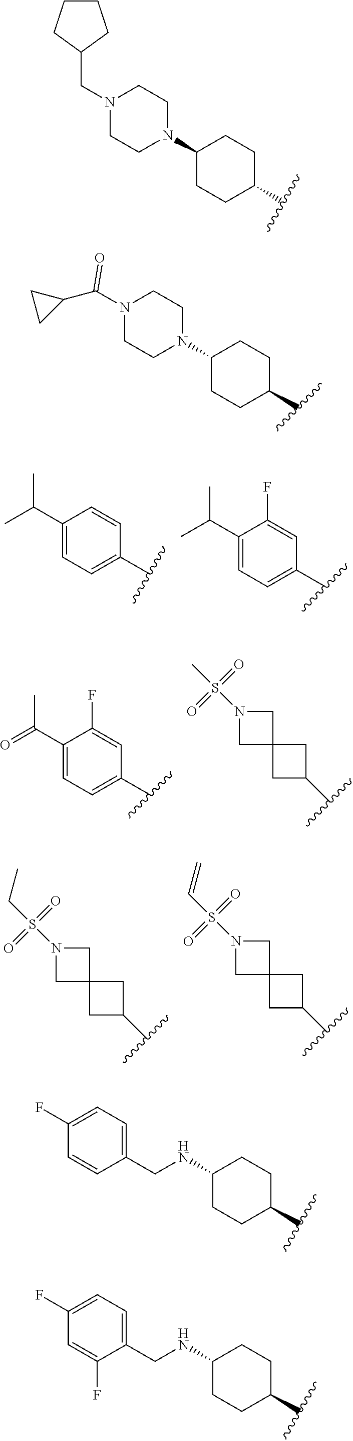 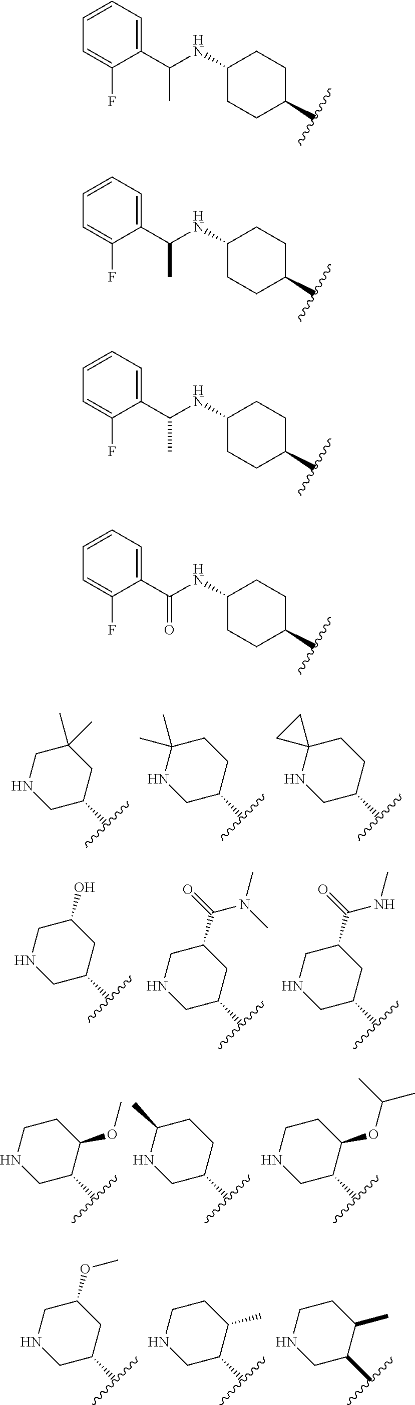 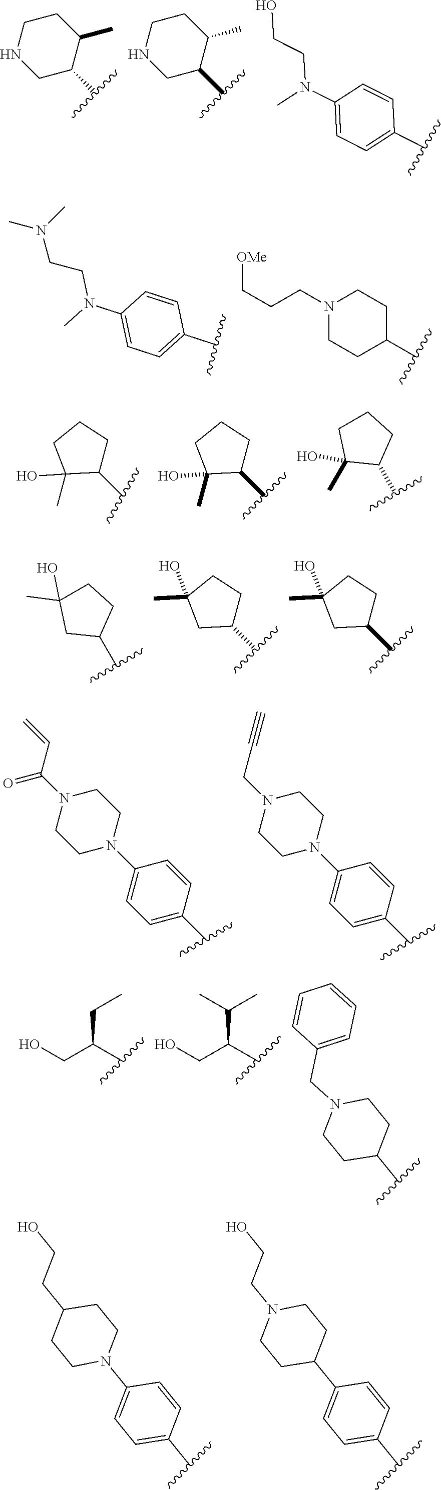 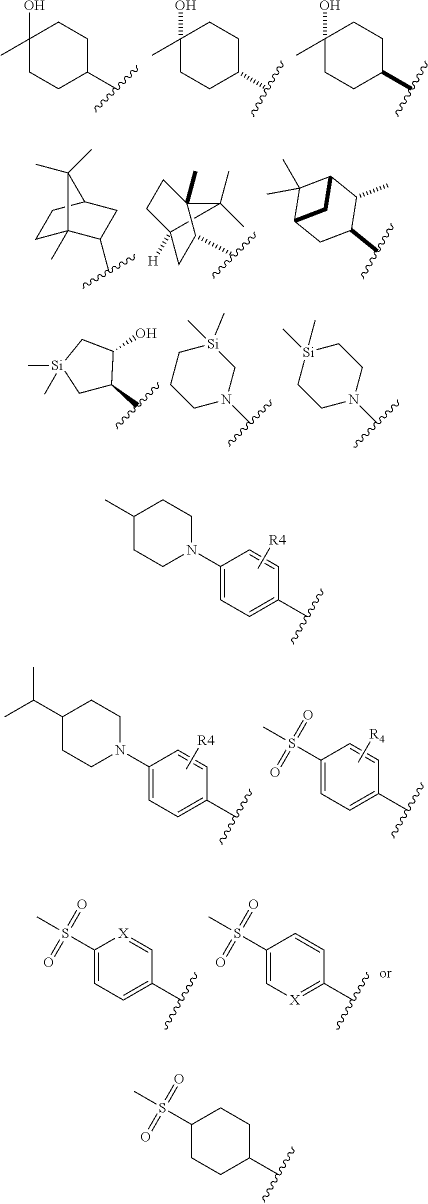
where R₃ is H, Me, isopropyl, isobutyl, CH₂CH₂OH, or bicyclo[1.1.1]pentane;
R₄ is H, Me, OMe, CF₃, or CHF₂;
R₅ is H or Me;
X is C—H, N, or C—F;
Y is H, N, F, Me, OMe, CF₃, or CHF₂;
Q is O or S;
n is 1, 2, or 3; and
R₂ is: 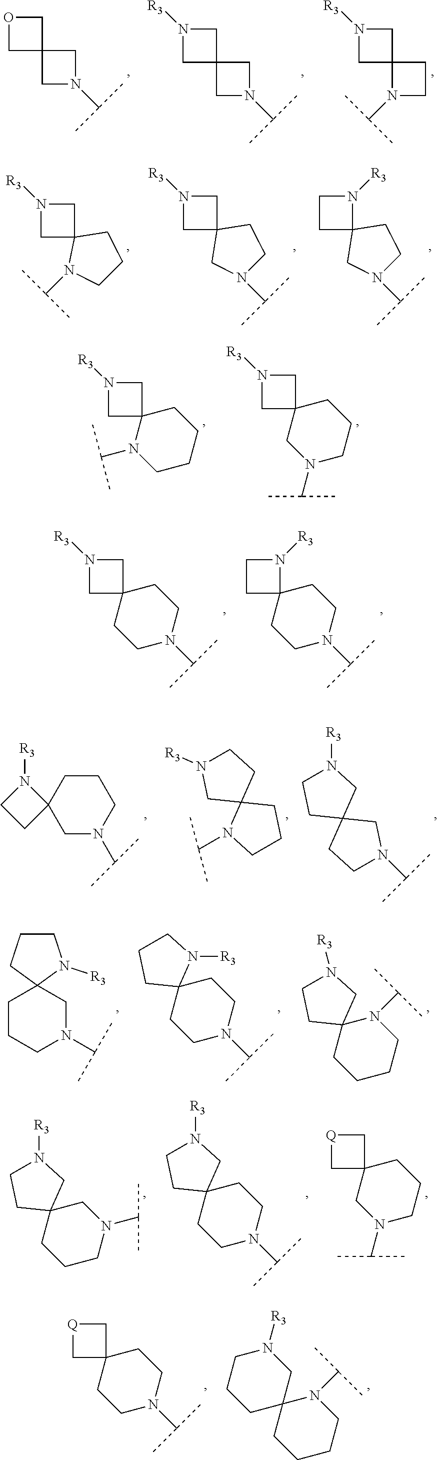 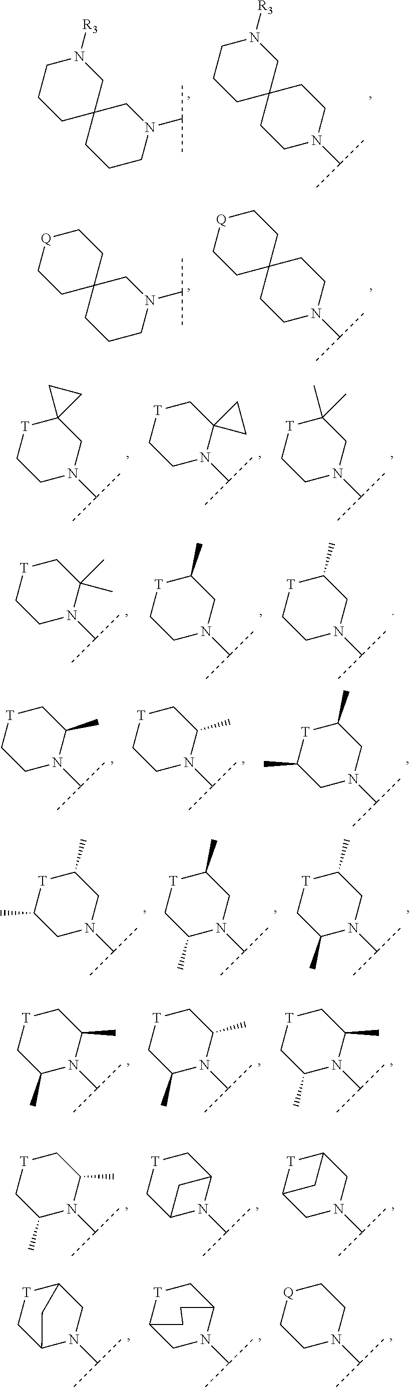 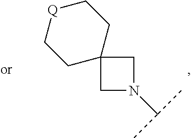
where T is O, NH, NMe, N-isopropyl, or N-isobutyl.

2. The compound of claim 1, wherein [symbol] is 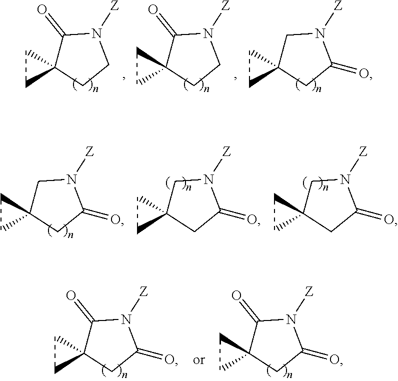
bonded in a spiro-manner, 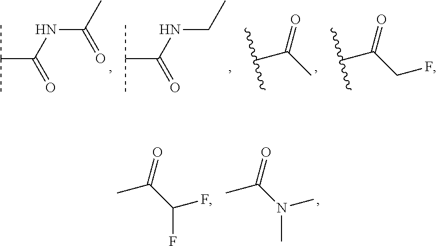
CN; where Z is H, Me, or CH₂CH₂OH, CH₂CH₂CN, CH₂CH₂F, COCH₃, COCH═CH; and n is 0, 1, 2, or 3.

4. The compound of claim 1, wherein R is

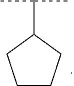

5. The compound of claim 1, wherein R₁ is: 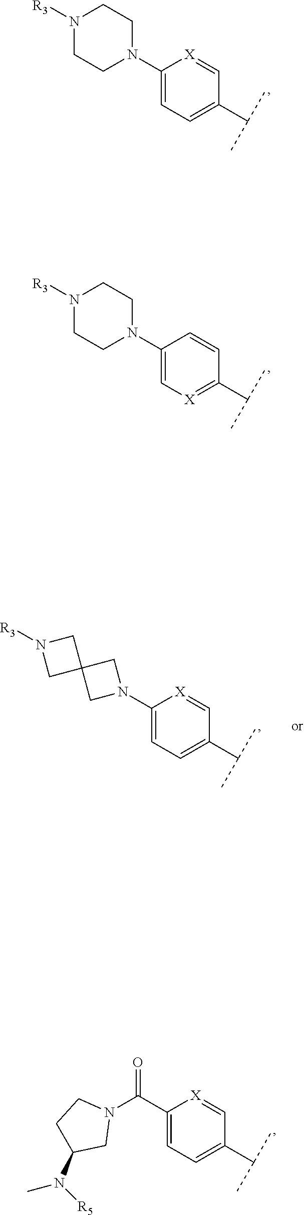
where R₃ is H, Me, isopropyl, isobutyl, CH₂CH₂OH, or bicyclo[1.1.1]pentane; R₅ is H or Me; and X is C—H, N, or C—F.

6. The compound of claim 1, wherein the compound comprises Formula I: 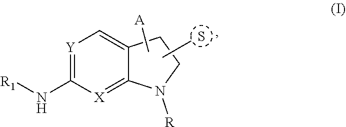
wherein A is none; and X and Y are independently, C or N.

7. The compound of claim 6, wherein [symbol] is 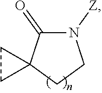
bonded in a spiro-manner; where Z is H, Me, or CH₂CH₂OH; and n is 0, 1, 2, or 3.

8. The compound of claim 6, wherein R is

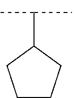

9. The compound of claim 6, wherein R₁ is: 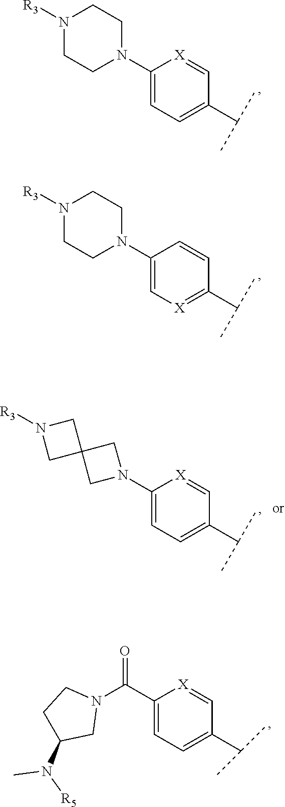
where R₃ is H, Me, isopropyl, isobutyl, CH₂CH₂OH, or bicyclo[1.1.1]pentane; H₅ is H or Me; and X is C—H, N, or C—F.

10. A compound or a pharmaceutically acceptable salt thereof comprising Formulae III-VIII: 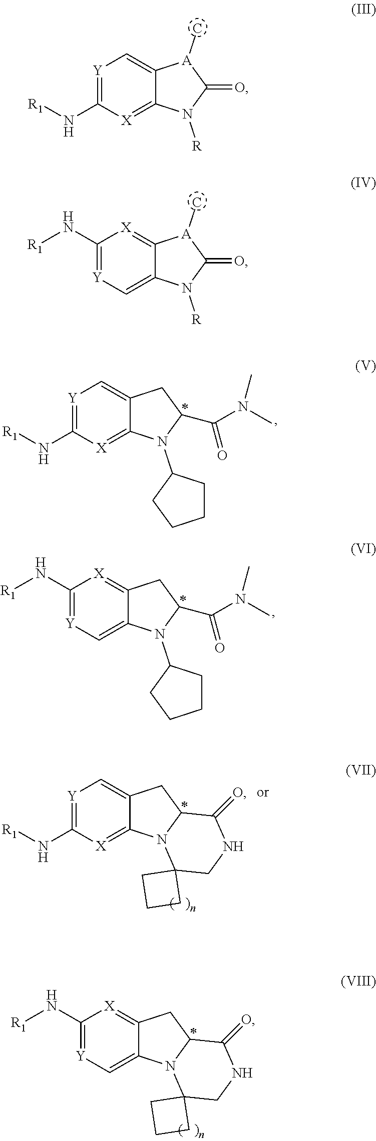
wherein: A is C or N;
X and Y are independently N, C, or C—F; [symbol]
is 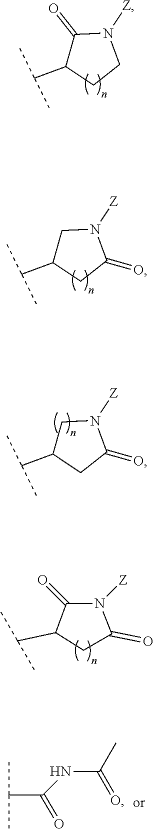 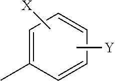
X=Y=H; X=Cl, F, Me, OMe, Y=H: X=Y=Cl, F, Me, OMe, where Z is H, Me, or CH₂CH₂OH; and n is 0, 1, 2, or 3;
R is (CH₂)ₙ, 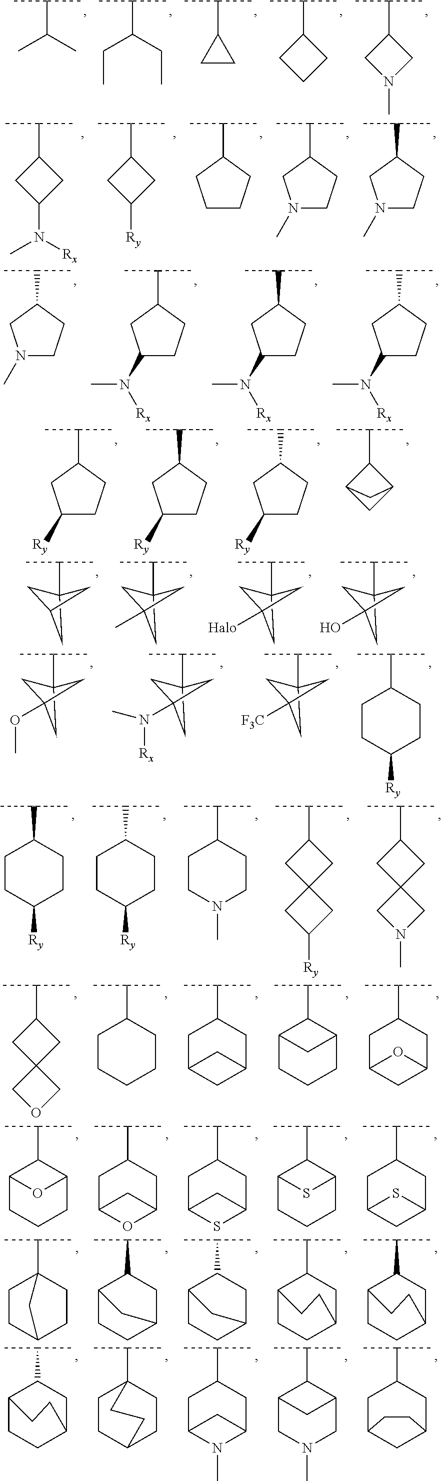 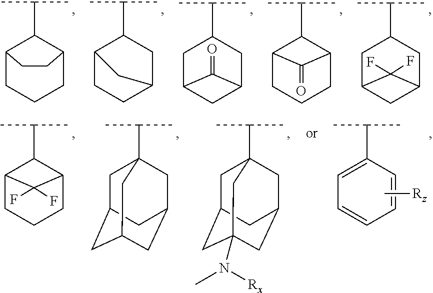
where Rₓ is H or Me, R_{y} is Me, OH, or OMe; R_{z} is H, Me, OMe, OH, N, F, or CF₃; and n is 1 to 11; and
R₁ is: 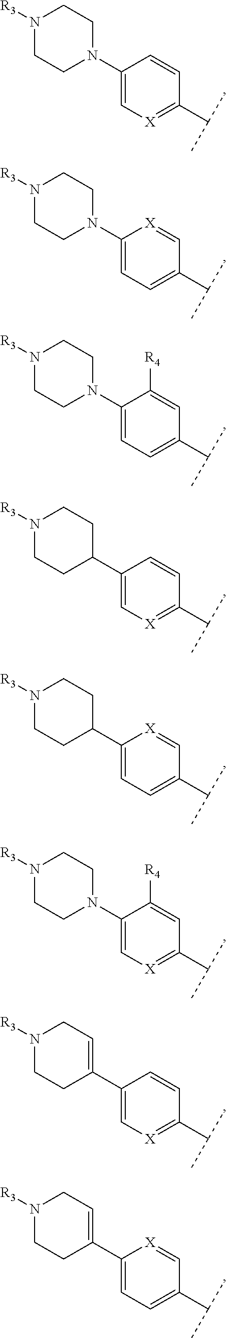 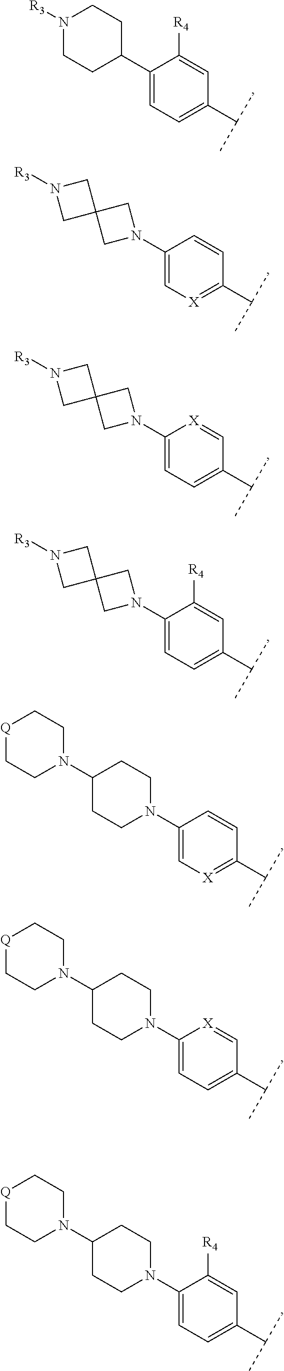 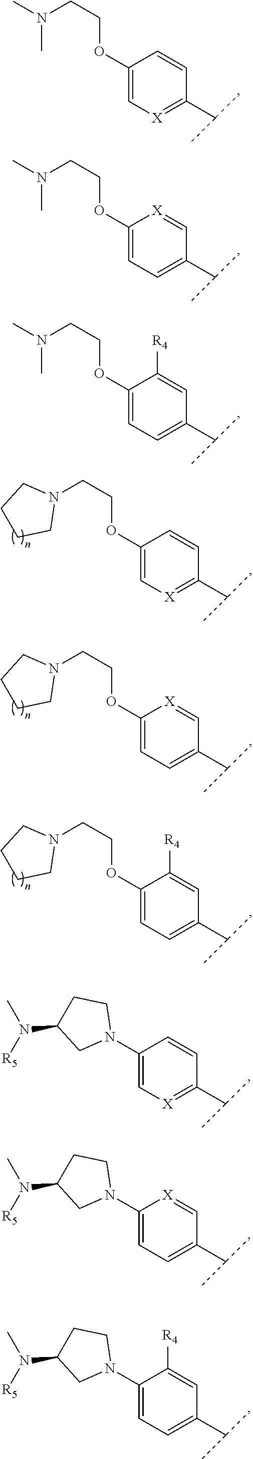 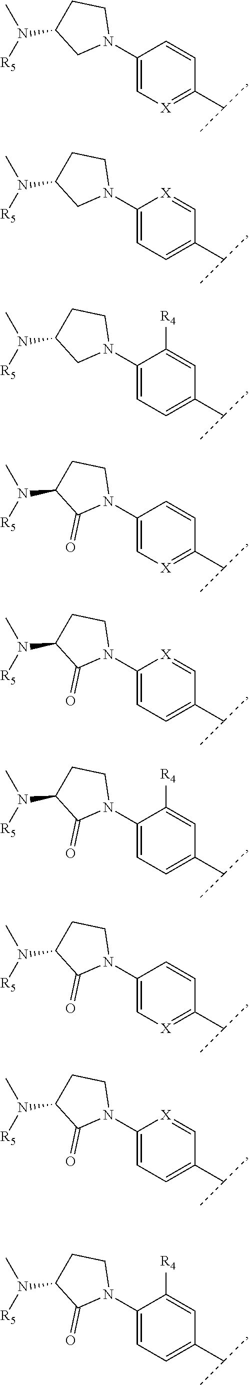 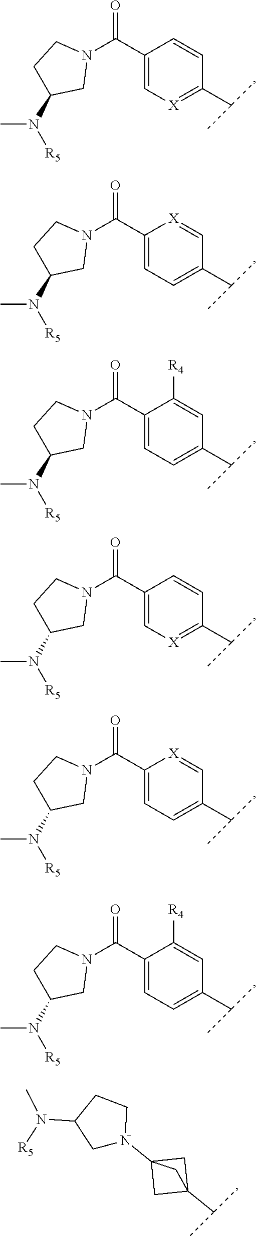 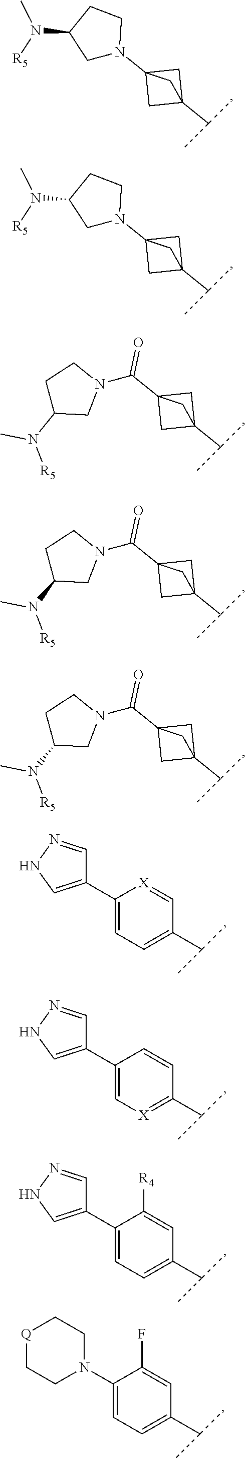 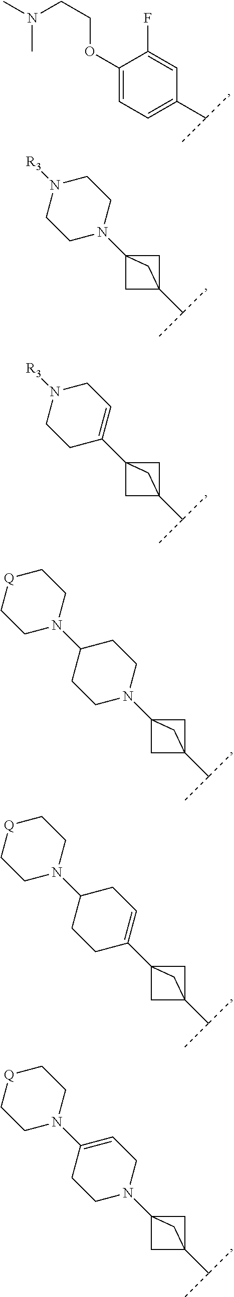 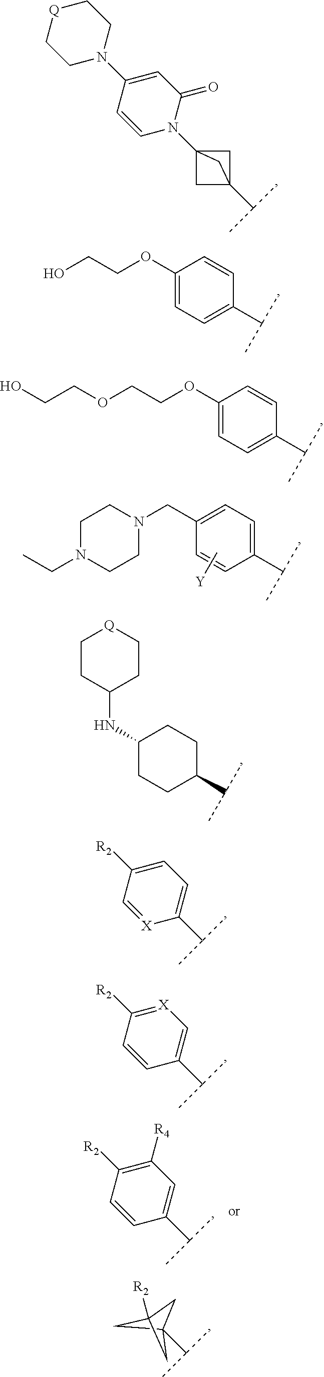
where R₃ is H, Me, isopropyl, isobutyl, CH₂CH₂OH, or bicyclo[1.1.1]pentane;
R₄ is H, Me, OMe, CF₃, or CHF₂;
R₅ is H or Me;
X is C—H, N, or C—F;
Y is H, N, F, Me, OMe, CF₃, or CHF₂;
Q is O or S;
n is 1, 2, or 3; and 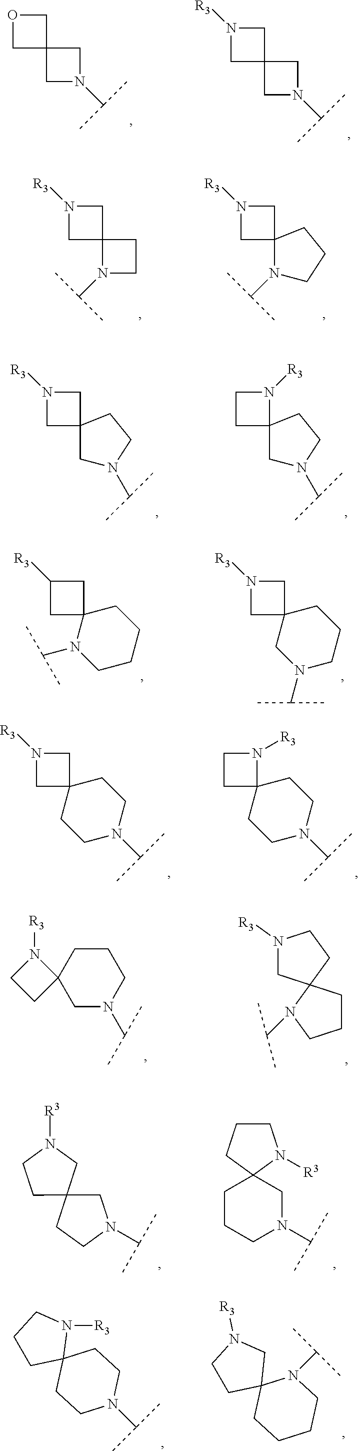 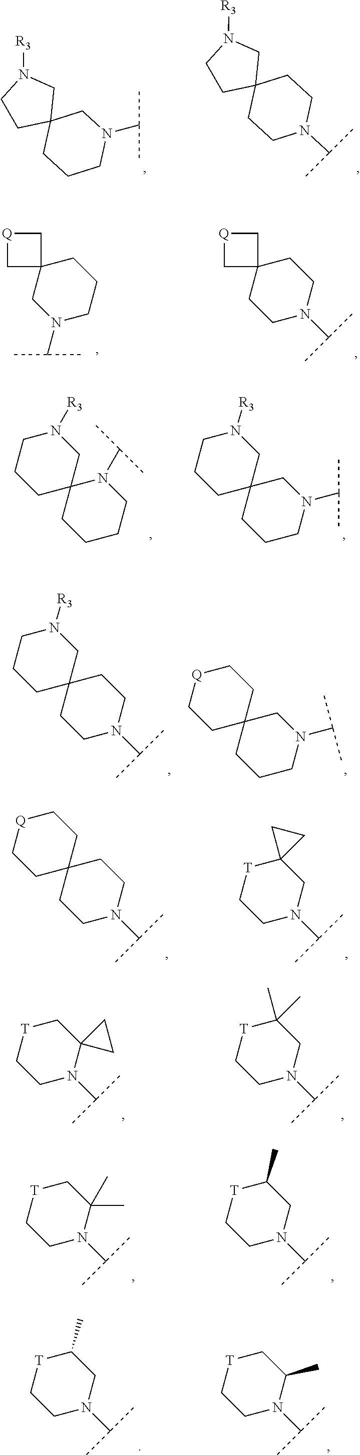 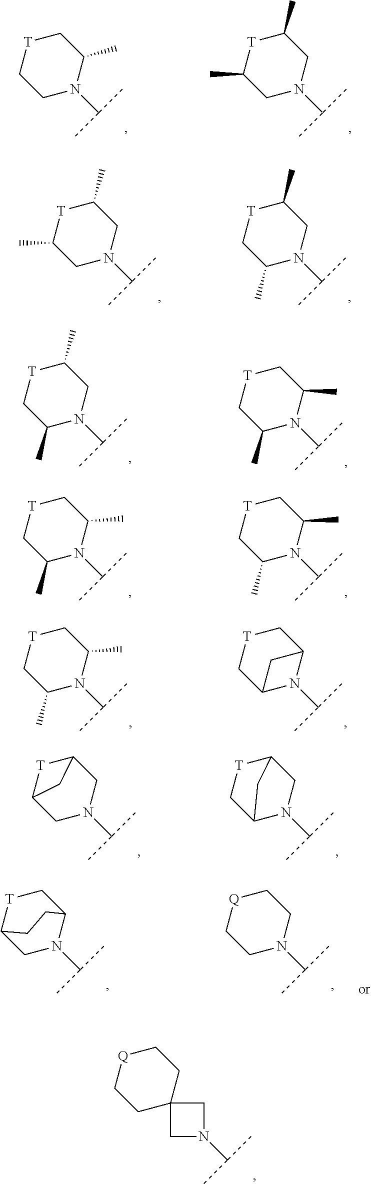
where T is O, NH, NMe, N-isopropyl, or N-isobutyl.

11. The compound of claim 10, wherein the compound comprises Formula (III): 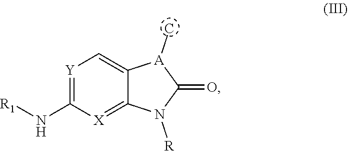
wherein: A is C or N; and X and Y are independently N, C, or C—F.

12. The compound of claim 10, wherein [symbol] is: 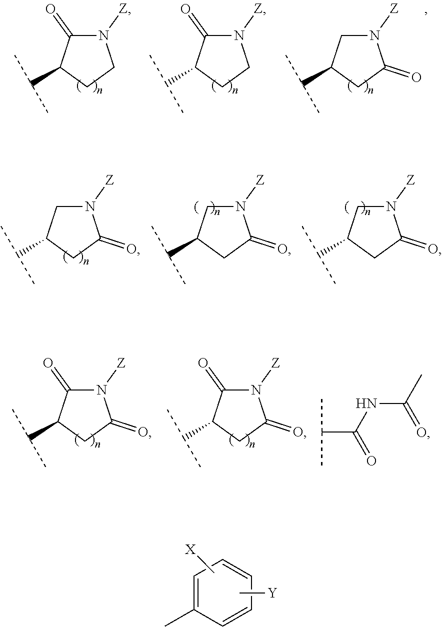
X=Y=H; X=Cl, F, Me, OMe, Y=H: X=Y=Cl, F, Me, OMe, where Z is H, Me, or CH₂CH₂OH; and n is 0, 1, 2, or 3.

13. The compound of claim 10, wherein R is

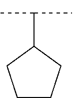

14. The compound of claim 10, wherein R₁ is: 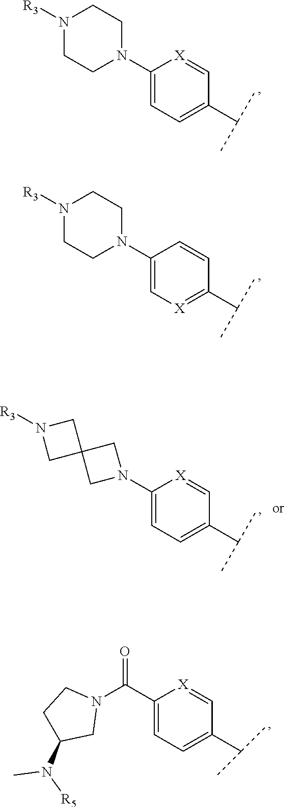
where R₃ is H, Me, isopropyl, isobutyl, CH₂CH₂OH, or bicyclo[1.1.1]pentane; R₅ is H or Me; and X is C—H, N, or C—F.

15. The compound of claim 10, wherein the compound comprises Formula (III): 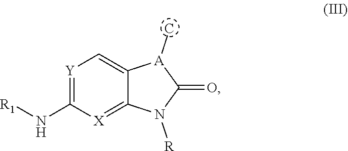
wherein: A is C;
X and Y are independently N; [symbol]
is: 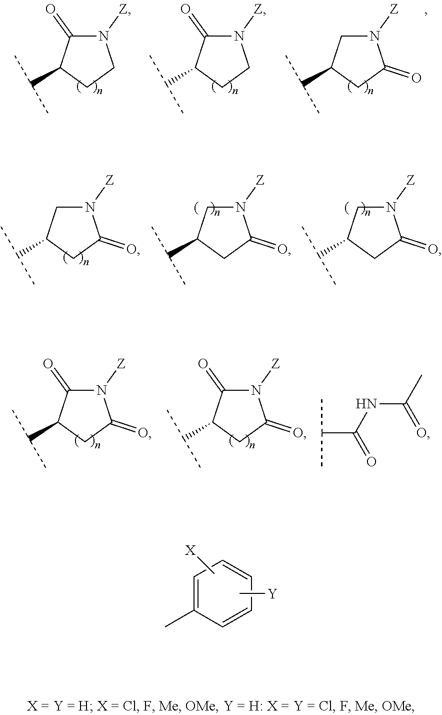
where Z is H, Me, or CH₂CH₂OH; and n is 0, 1, 2, or 3;
R is 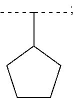
and
R₁ is: 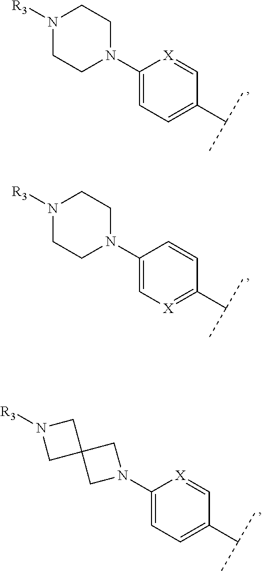
or 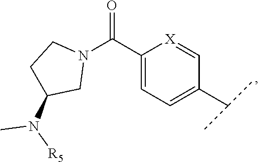
where R₃ is H, Me, isopropyl, isobutyl, CH₂CH₂OH, or bicyclo[1.1.1]pentane; R₅ is H or Me; and X is C—H, N, or C—F.

16. A compound or a pharmaceutically acceptable salt thereof comprising Formulae IX-XX: 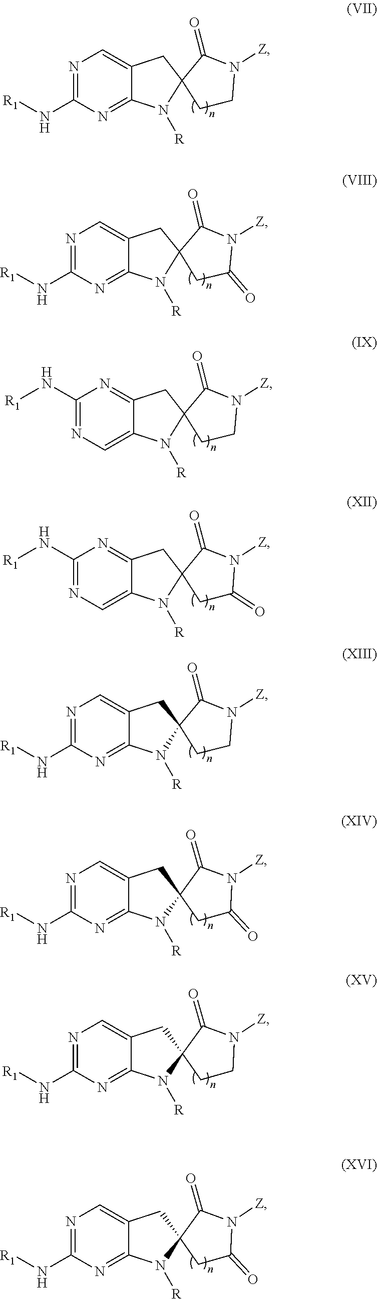 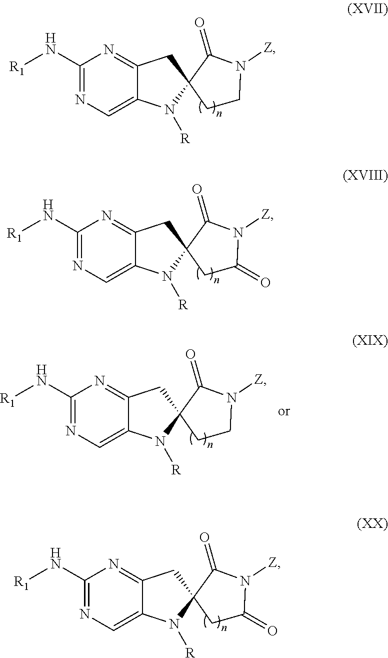
where Z is H, Me, or CH₂CH₂OH, and n is 0, 1, 2, or 3;
R is (CH₂)ₙ, 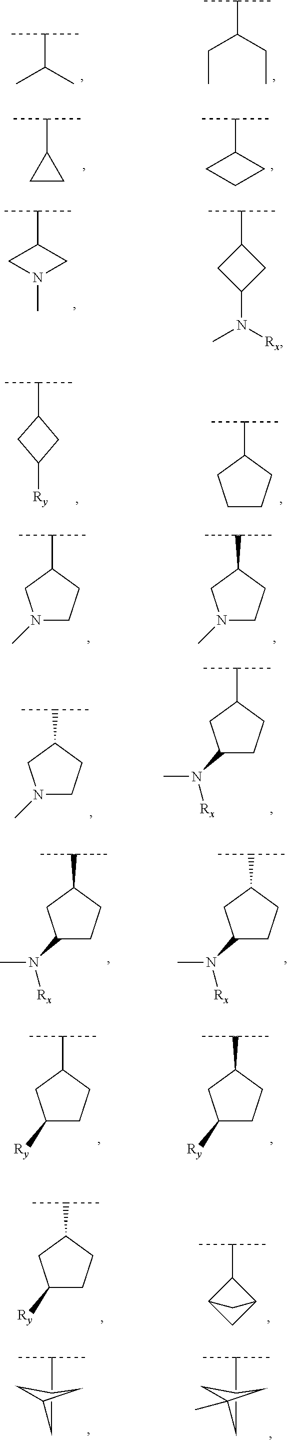 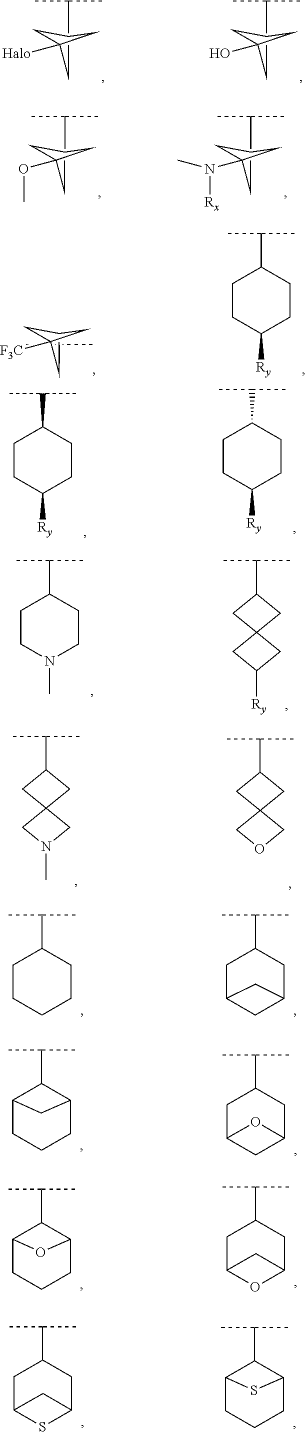 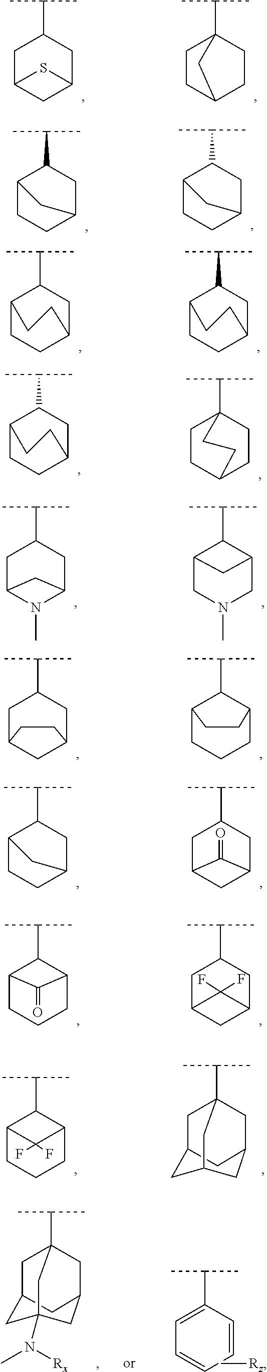
where Rₓ is H or Me, R_{y} is Me, OH, or OMe; R_{z} is H, Me, OMe, OH, N, F, or CF₃; and n is 1 to 11; and
R₁ is 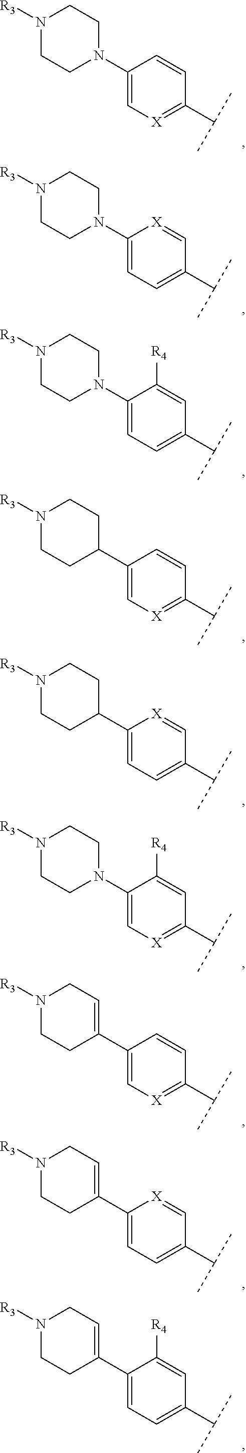 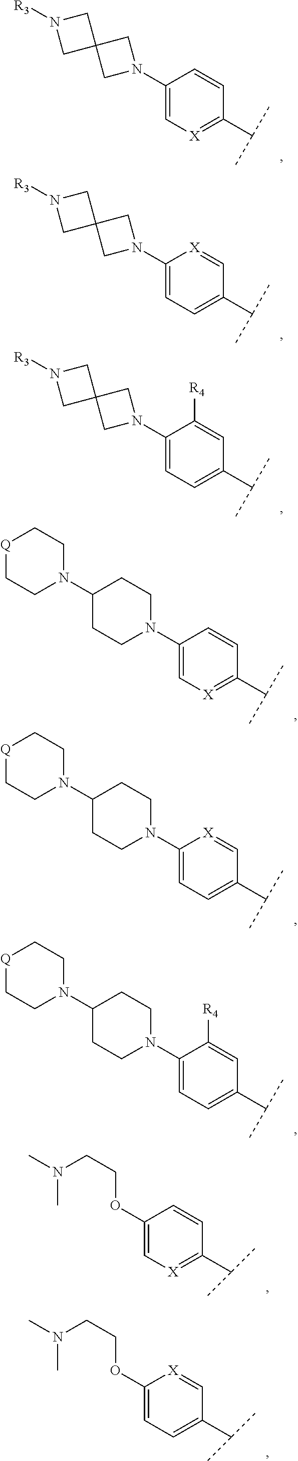 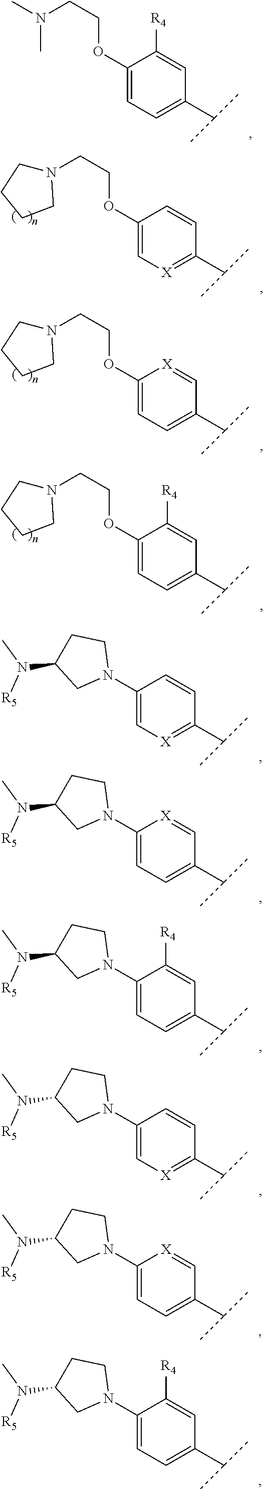 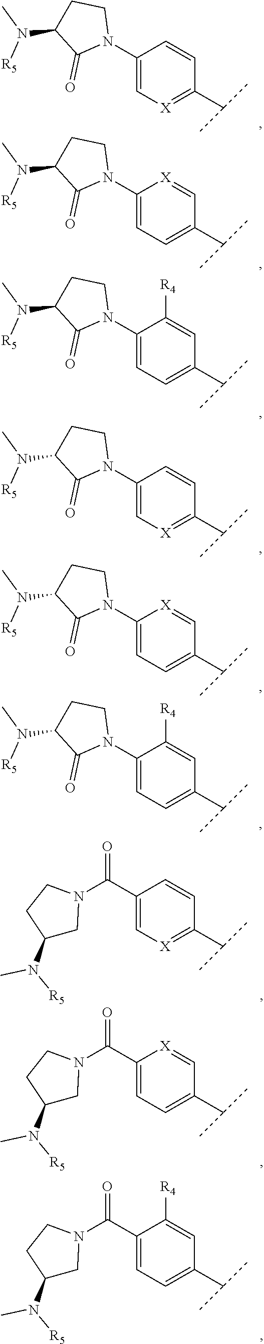 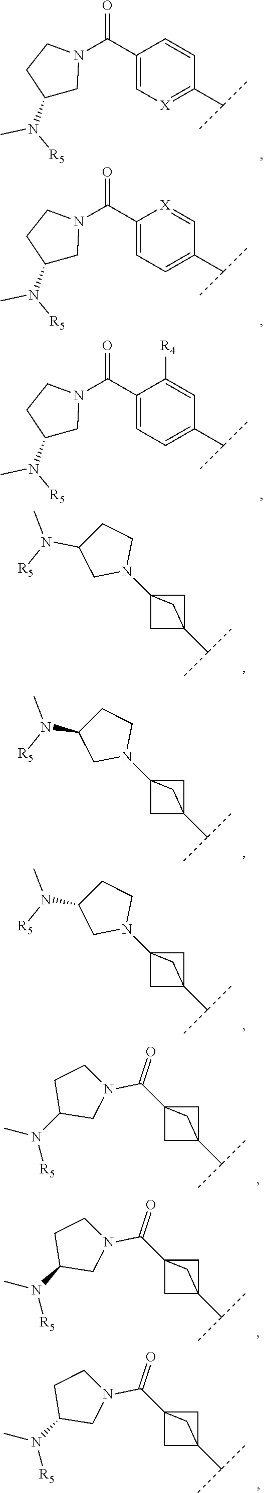 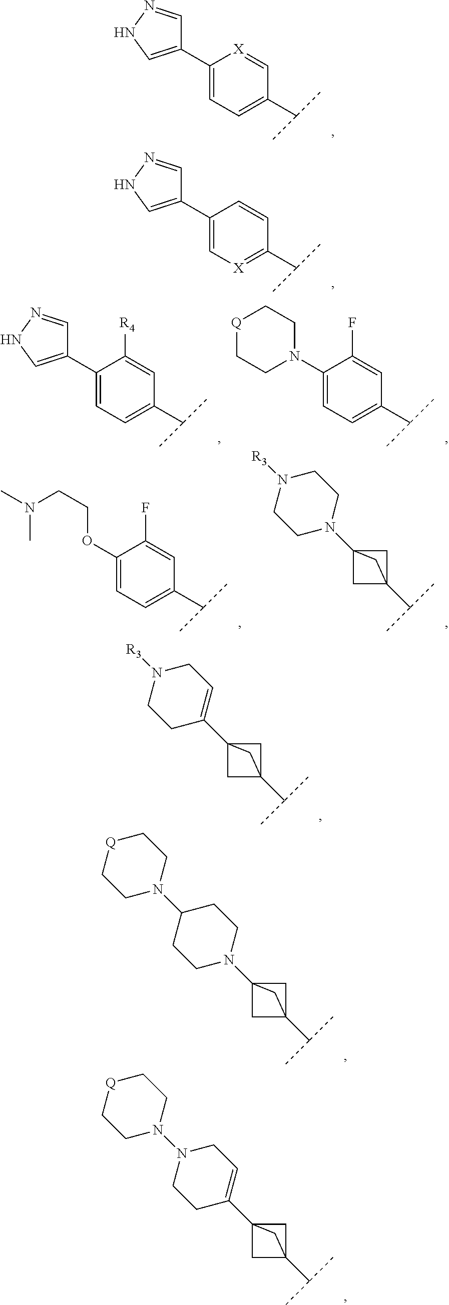 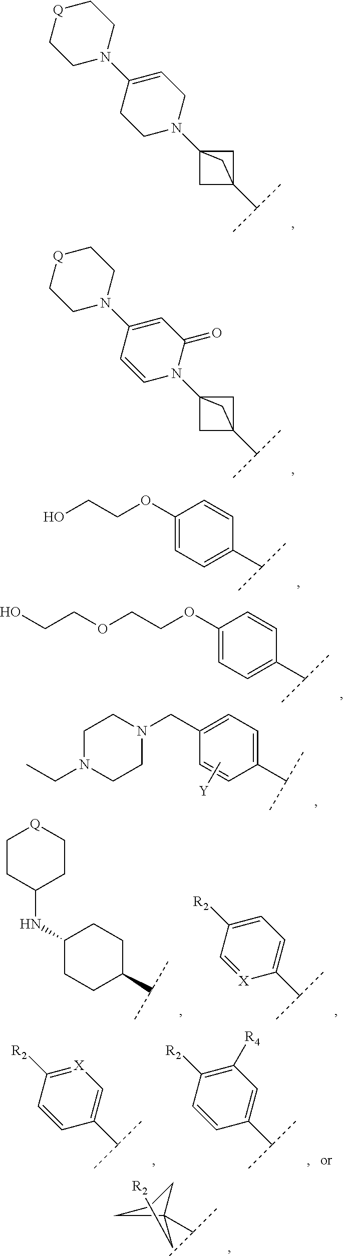
where R₃ is H, Me, isopropyl, isobutyl, CH₂CH₂OH, or bicyclo[1.1.1]pentane;
R₄ is H, Me, OMe, CF₃, or CHF₂;
R₅ is H or Me;
X is C—H, N, or C—F;
Y is H, N, F, Me, OMe, CF₃, or CHF₂;
Q is O or S;
n is 1, 2, or 3; and
R₂ is: 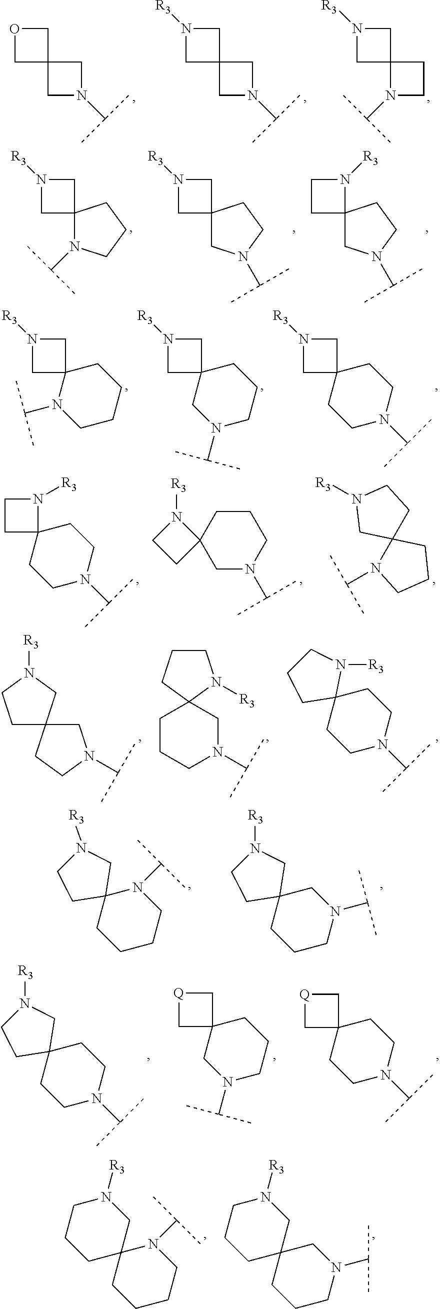 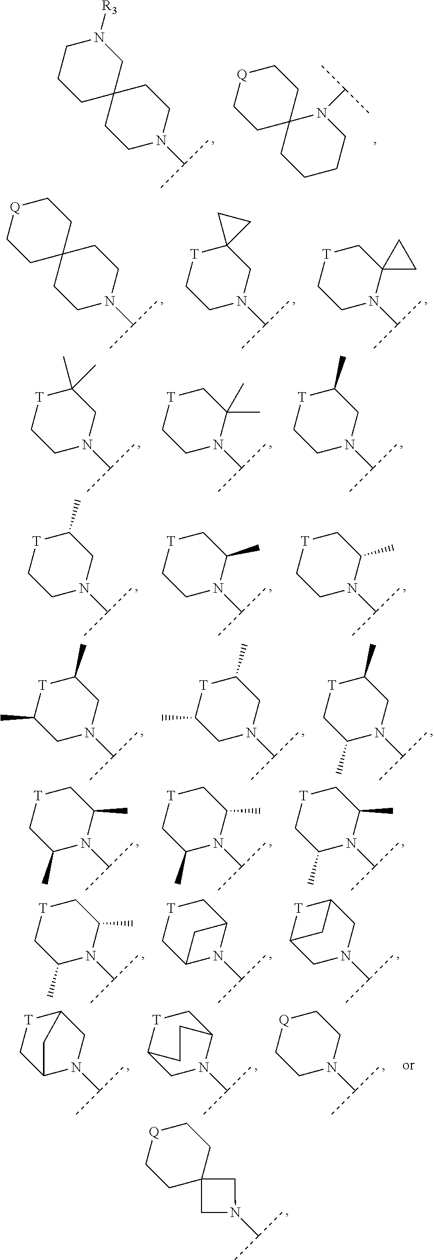
where T is O, NH, NMe, N-isopropyl, or N-isobutyl.

17. The compound of claim 16, wherein the compound comprises Formulae (IX), (XIII), or (XV):

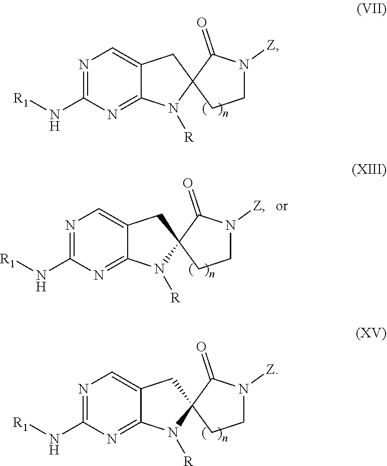

18. The compound of claim 17, wherein R is

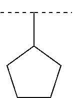

19. The compound of claim 17, wherein R₁ is: 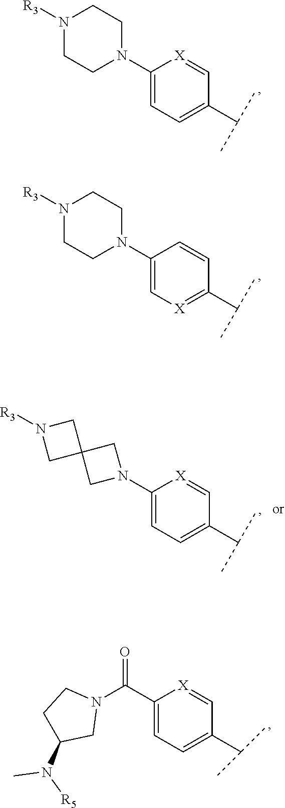
where R₃ is H, Me, isopropyl, isobutyl, CH₂CH₂OH, or bicyclo[1.1.1]pentane; R₅ is H or Me; and X is C—H, N, or C—F.

20. A compound selected from C1-C210 or pharmaceutically acceptable salts thereof:

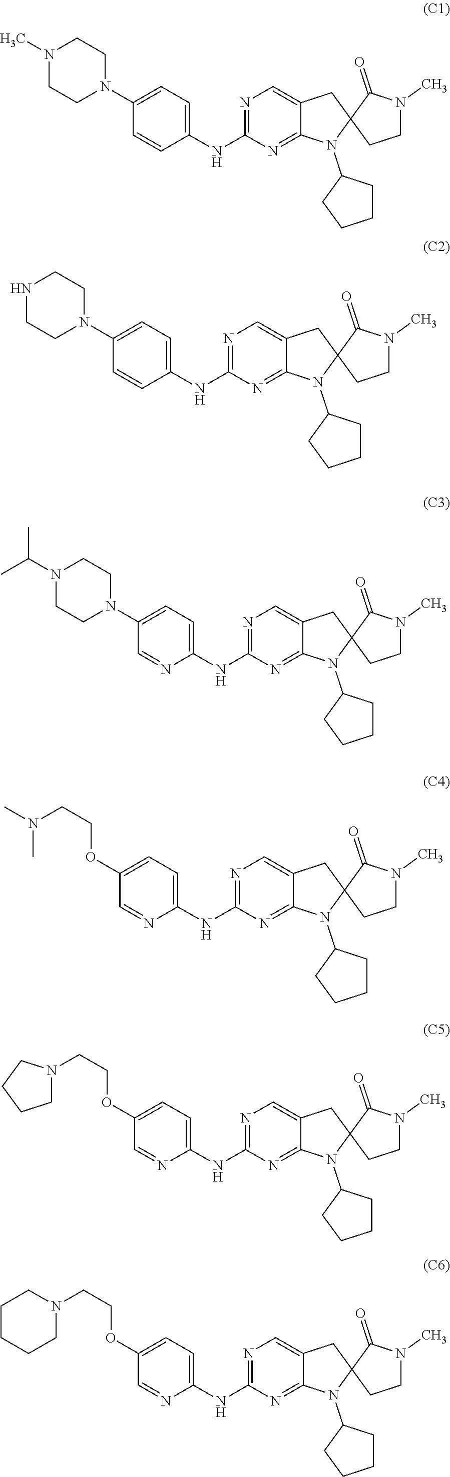

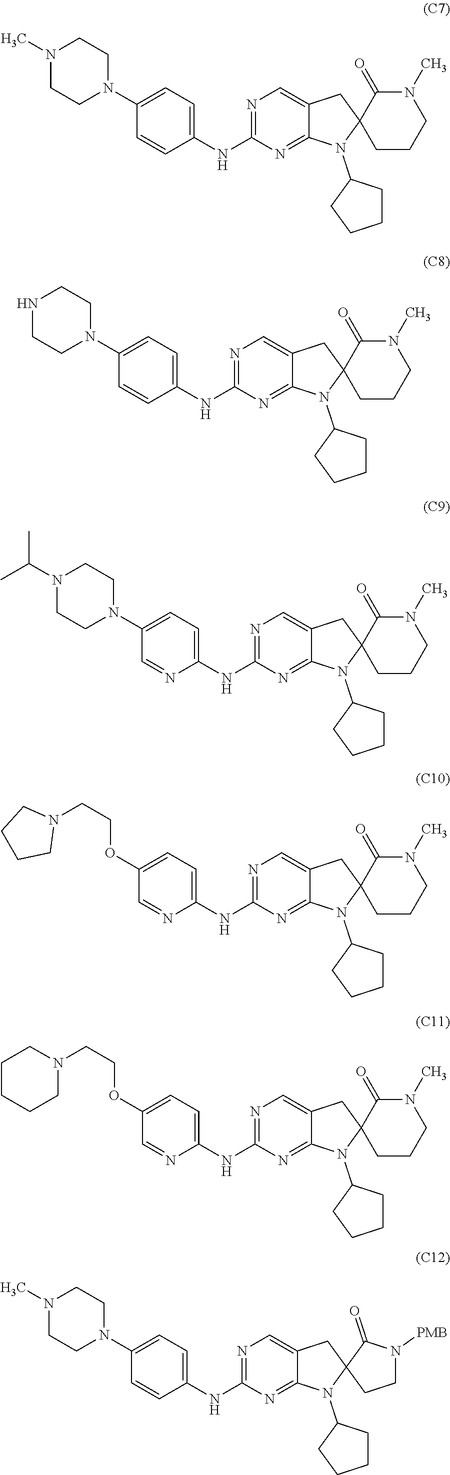

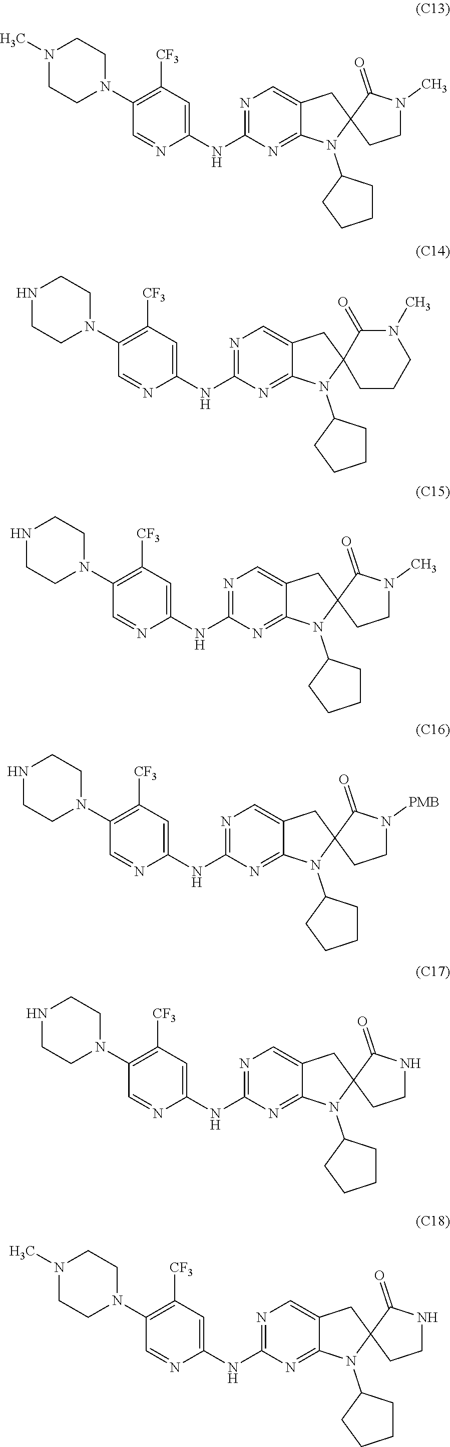

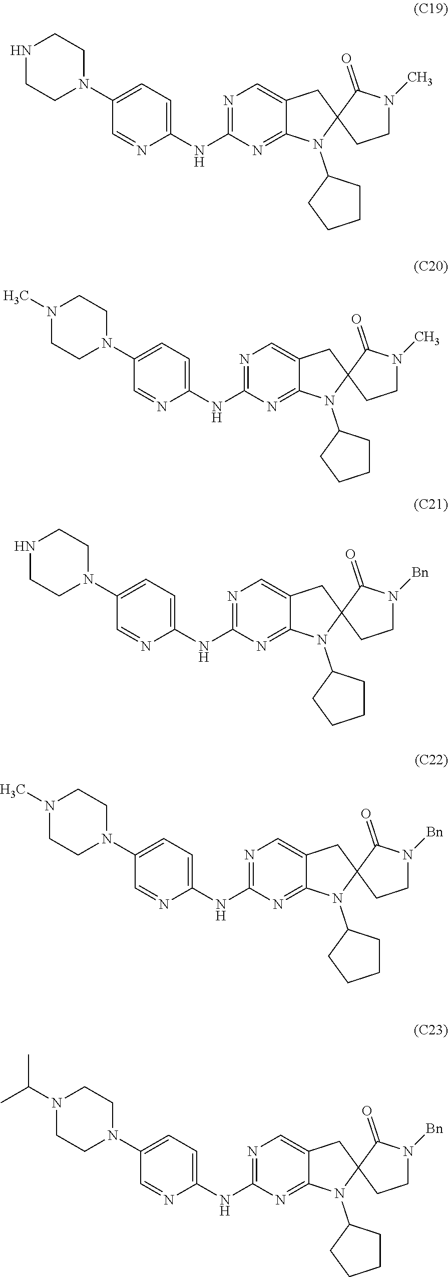

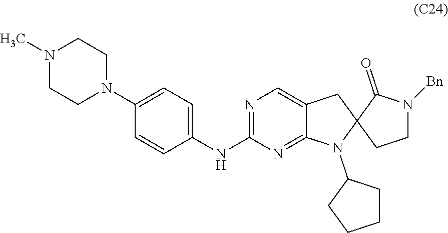 [SMILES: CN1CCN(c2ccc(Nc3ncc4c(n3)N(C3CCCC3)C3(CCN(Cc5ccccc5)C3=O)C4)cc2)CC1]

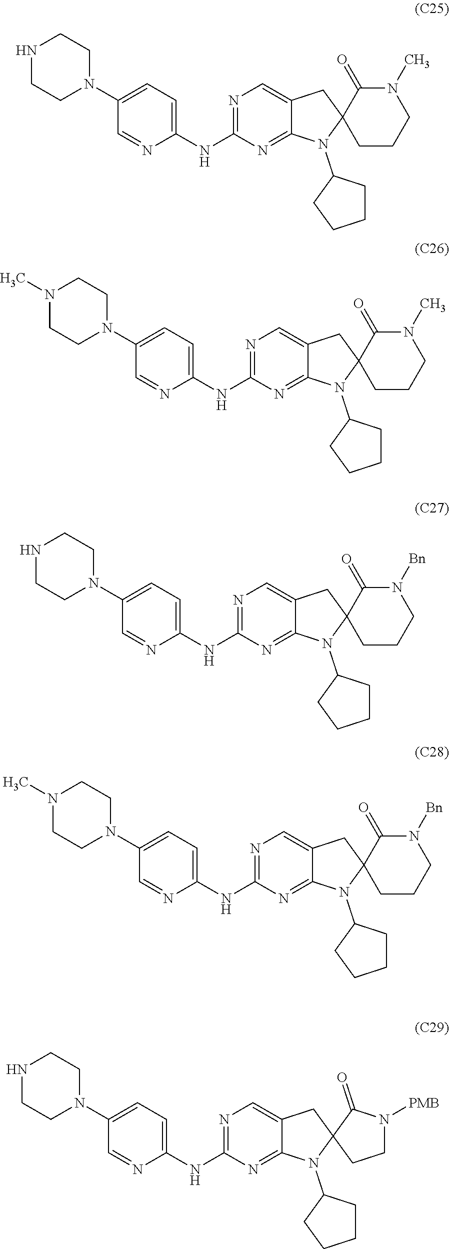

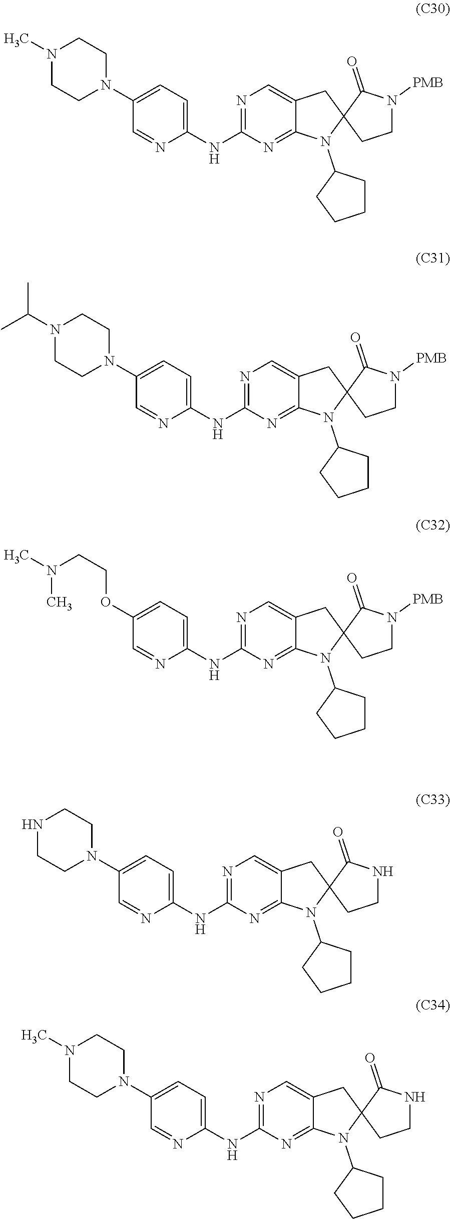

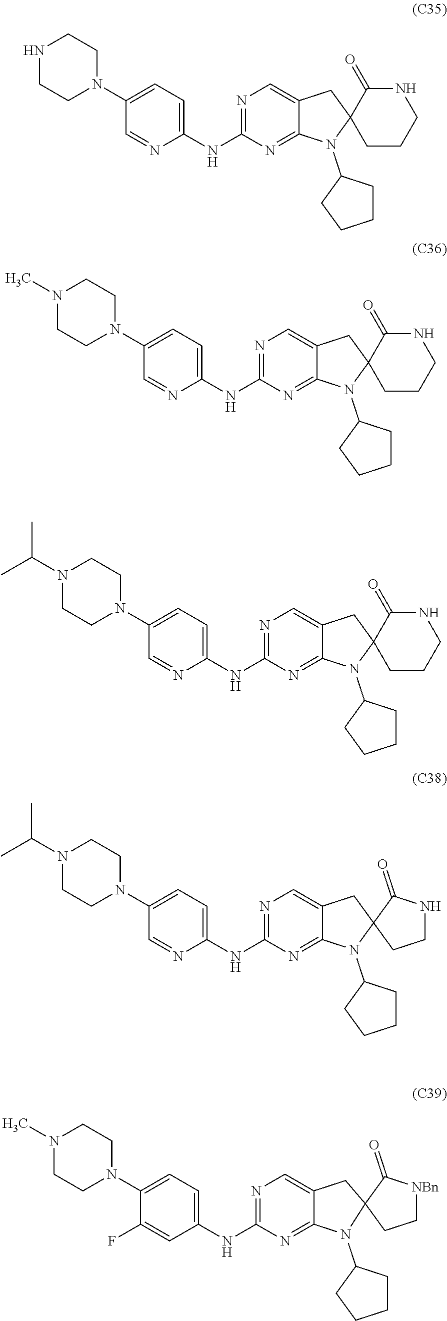 [SMILES: O=C1NCCCC12Cc1cnc(Nc3ccc(N4CCNCC4)cn3)nc1N2C1CCCC1 ; CN1CCN(c2ccc(Nc3ncc4c(n3)N(C3CCCC3)C3(CCCNC3=O)C4)nc2)CC1 ; CC(C)N1CCN(c2ccc(Nc3ncc4c(n3)N(C3CCCC3)C3(CCCNC3=O)C4)nc2)CC1 ; CC(C)N1CCN(c2ccc(Nc3ncc4c(n3)N(C3CCCC3)C3(CCNC3=O)C4)nc2)CC1 ; CN1CCN(c2ccc(Nc3ncc4c(n3)N(C3CCCC3)C3(CCN(Cc5ccccc5)C3=O)C4)cc2F)CC1]

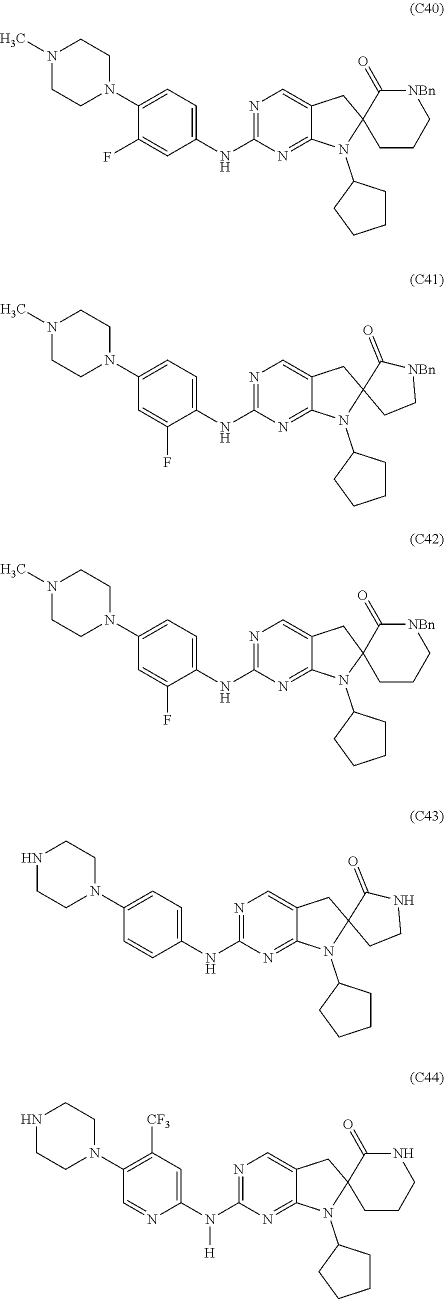 [SMILES: CN1CCN(c2ccc(Nc3ncc4c(n3)N(C3CCCC3)C3(CCCN(Cc5ccccc5)C3=O)C4)cc2F)CC1 ; CN1CCN(c2ccc(Nc3ncc4c(n3)N(C3CCCC3)C3(CCN(Cc5ccccc5)C3=O)C4)c(F)c2)CC1 ; CN1CCN(c2ccc(Nc3ncc4c(n3)N(C3CCCC3)C3(CCCN(Cc5ccccc5)C3=O)C4)c(F)c2)CC1 ; O=C1NCCC12Cc1cnc(Nc3ccc(N4CCNCC4)cc3)nc1N2C1CCCC1 ; O=C1NCCCC12Cc1cnc(Nc3cc(C(F)(F)F)c(N4CCNCC4)cn3)nc1N2C1CCCC1]

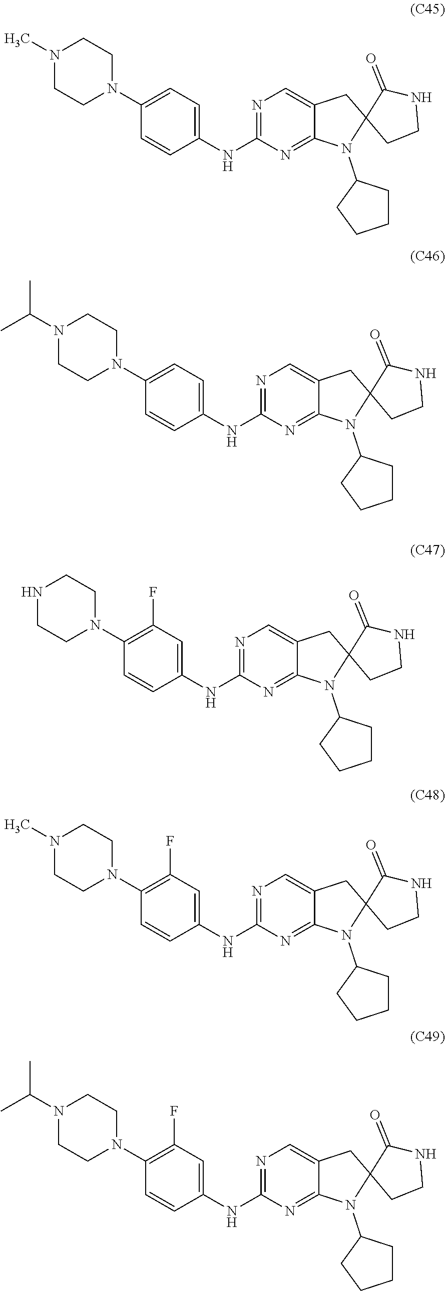 [SMILES: CN1CCN(c2ccc(Nc3ncc4c(n3)N(C3CCCC3)C3(CCNC3=O)C4)cc2)CC1 ; CC(C)N1CCN(c2ccc(Nc3ncc4c(n3)N(C3CCCC3)C3(CCNC3=O)C4)cc2)CC1 ; O=C1NCCC12Cc1cnc(Nc3ccc(N4CCNCC4)c(F)c3)nc1N2C1CCCC1 ; CN1CCN(c2ccc(Nc3ncc4c(n3)N(C3CCCC3)C3(CCNC3=O)C4)cc2F)CC1 ; CC(C)N1CCN(c2ccc(Nc3ncc4c(n3)N(C3CCCC3)C3(CCNC3=O)C4)cc2F)CC1]

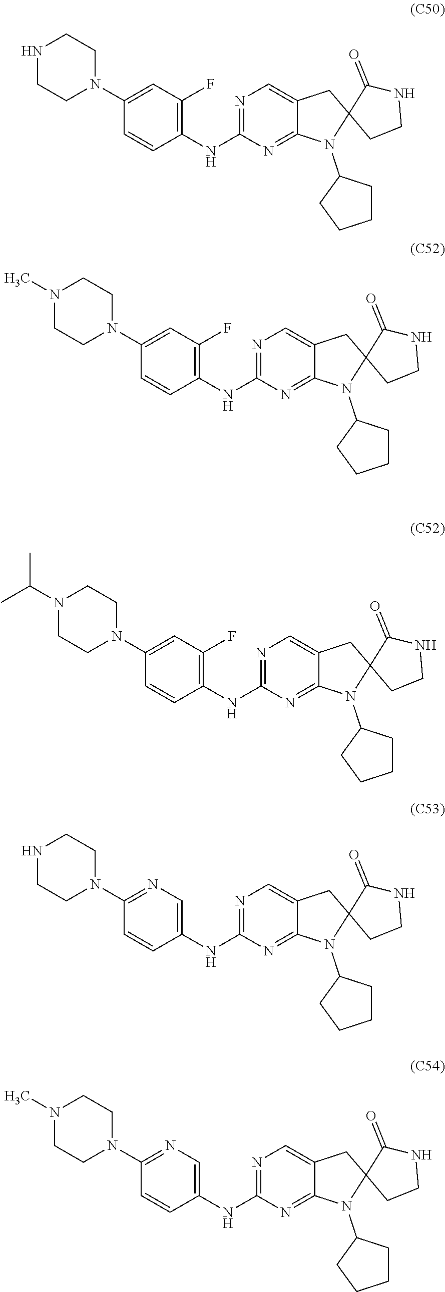 [SMILES: O=C1NCCC12Cc1cnc(Nc3ccc(N4CCNCC4)cc3F)nc1N2C1CCCC1 ; CN1CCN(c2ccc(Nc3ncc4c(n3)N(C3CCCC3)C3(CCNC3=O)C4)c(F)c2)CC1 ; CC(C)N1CCN(c2ccc(Nc3ncc4c(n3)N(C3CCCC3)C3(CCNC3=O)C4)c(F)c2)CC1 ; O=C1NCCC12Cc1cnc(Nc3ccc(N4CCNCC4)nc3)nc1N2C1CCCC1 ; CN1CCN(c2ccc(Nc3ncc4c(n3)N(C3CCCC3)C3(CCNC3=O)C4)cn2)CC1]

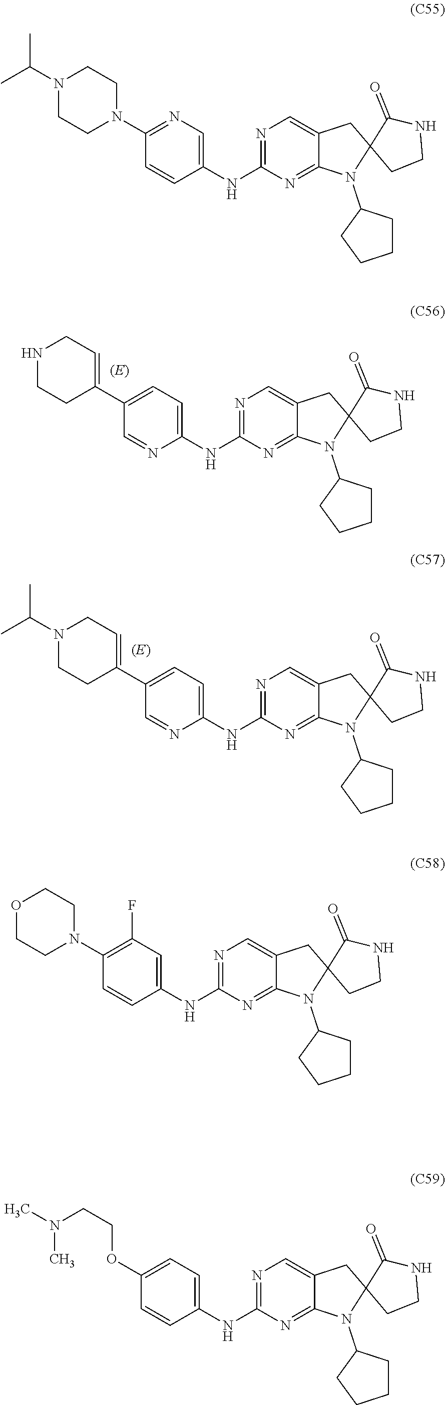 [SMILES: CC(C)N1CCN(c2ccc(Nc3ncc4c(n3)N(C3CCCC3)C3(CCNC3=O)C4)cn2)CC1 ; O=C1NCCC12Cc1cnc(Nc3ccc(C4=CCNCC4)cn3)nc1N2C1CCCC1 ; CC(C)N1CC=C(c2ccc(Nc3ncc4c(n3)N(C3CCCC3)C3(CCNC3=O)C4)nc2)CC1 ; O=C1NCCC12Cc1cnc(Nc3ccc(N4CCOCC4)c(F)c3)nc1N2C1CCCC1 ; CN(C)CCOc1ccc(Nc2ncc3c(n2)N(C2CCCC2)C2(CCNC2=O)C3)cc1]

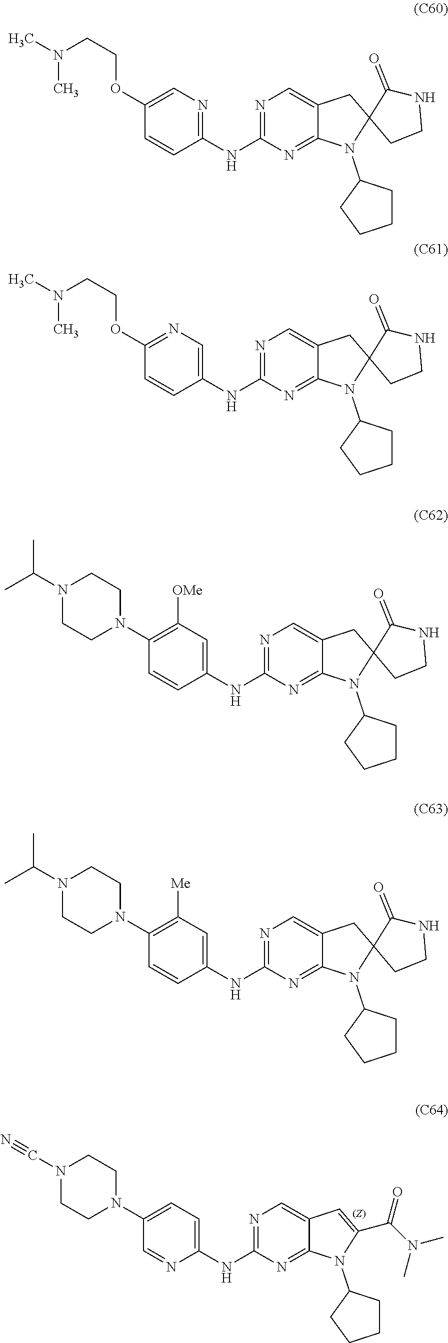 [SMILES: CN(C)CCOc1ccc(Nc2ncc3c(n2)N(C2CCCC2)C2(CCNC2=O)C3)nc1 ; CN(C)CCOc1ccc(Nc2ncc3c(n2)N(C2CCCC2)C2(CCNC2=O)C3)cn1 ; COc1cc(Nc2ncc3c(n2)N(C2CCCC2)C2(CCNC2=O)C3)ccc1N1CCN(C(C)C)CC1 ; Cc1cc(Nc2ncc3c(n2)N(C2CCCC2)C2(CCNC2=O)C3)ccc1N1CCN(C(C)C)CC1 ; CN(C)C(=O)c1cc2cnc(Nc3ccc(N4CCN(C#N)CC4)cn3)nc2n1C1CCCC1]

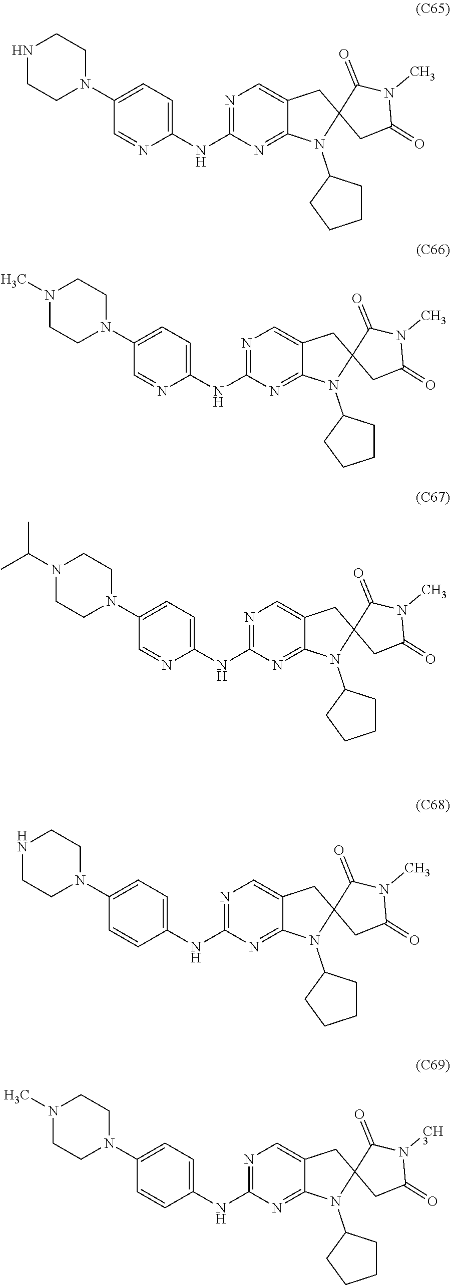 [SMILES: CN1C(=O)CC2(Cc3cnc(Nc4ccc(N5CCNCC5)cn4)nc3N2C2CCCC2)C1=O ; CN1CCN(c2ccc(Nc3ncc4c(n3)N(C3CCCC3)C3(CC(=O)N(C)C3=O)C4)nc2)CC1 ; CC(C)N1CCN(c2ccc(Nc3ncc4c(n3)N(C3CCCC3)C3(CC(=O)N(C)C3=O)C4)nc2)CC1 ; CN1C(=O)CC2(Cc3cnc(Nc4ccc(N5CCNCC5)cc4)nc3N2C2CCCC2)C1=O ; CN1CCN(c2ccc(Nc3ncc4c(n3)N(C3CCCC3)C3(CC(=O)N(C)C3=O)C4)cc2)CC1]

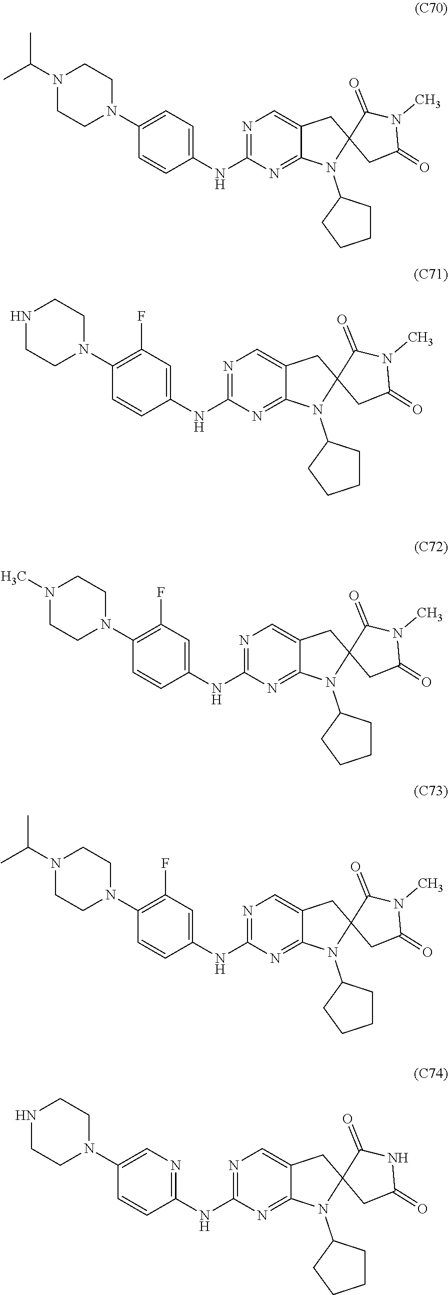 [SMILES: CC(C)N1CCN(c2ccc(Nc3ncc4c(n3)N(C3CCCC3)C3(CC(=O)N(C)C3=O)C4)cc2)CC1 ; CN1C(=O)CC2(Cc3cnc(Nc4ccc(N5CCNCC5)c(F)c4)nc3N2C2CCCC2)C1=O ; CN1CCN(c2ccc(Nc3ncc4c(n3)N(C3CCCC3)C3(CC(=O)N(C)C3=O)C4)cc2F)CC1 ; CC(C)N1CCN(c2ccc(Nc3ncc4c(n3)N(C3CCCC3)C3(CC(=O)N(C)C3=O)C4)cc2F)CC1 ; O=C1CC2(Cc3cnc(Nc4ccc(N5CCNCC5)cn4)nc3N2C2CCCC2)C(=O)N1]

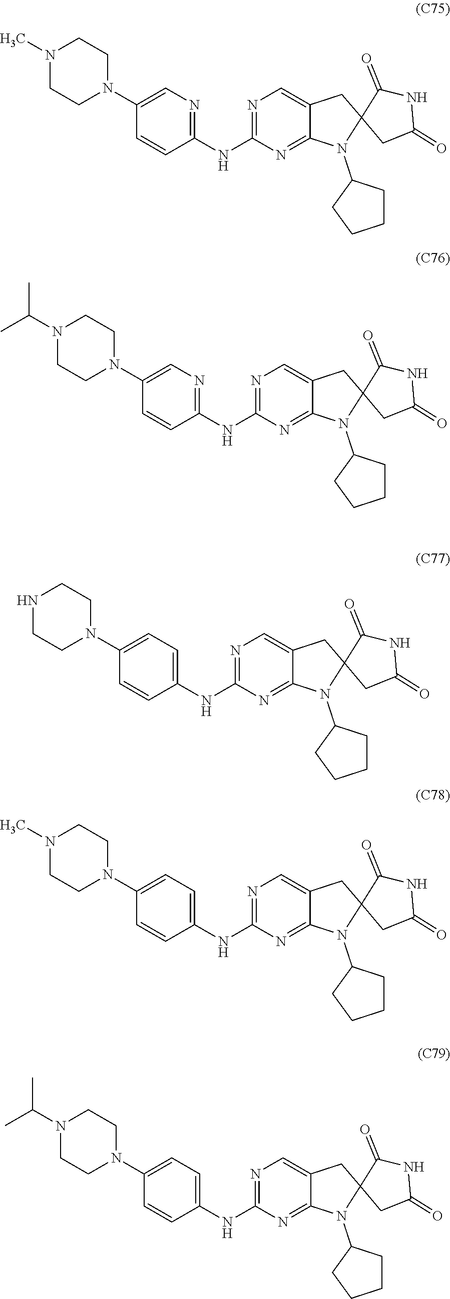 [SMILES: CN1CCN(c2ccc(Nc3ncc4c(n3)N(C3CCCC3)C3(CC(=O)NC3=O)C4)nc2)CC1 ; CC(C)N1CCN(c2ccc(Nc3ncc4c(n3)N(C3CCCC3)C3(CC(=O)NC3=O)C4)nc2)CC1 ; O=C1CC2(Cc3cnc(Nc4ccc(N5CCNCC5)cc4)nc3N2C2CCCC2)C(=O)N1 ; CN1CCN(c2ccc(Nc3ncc4c(n3)N(C3CCCC3)C3(CC(=O)NC3=O)C4)cc2)CC1 ; CC(C)N1CCN(c2ccc(Nc3ncc4c(n3)N(C3CCCC3)C3(CC(=O)NC3=O)C4)cc2)CC1]

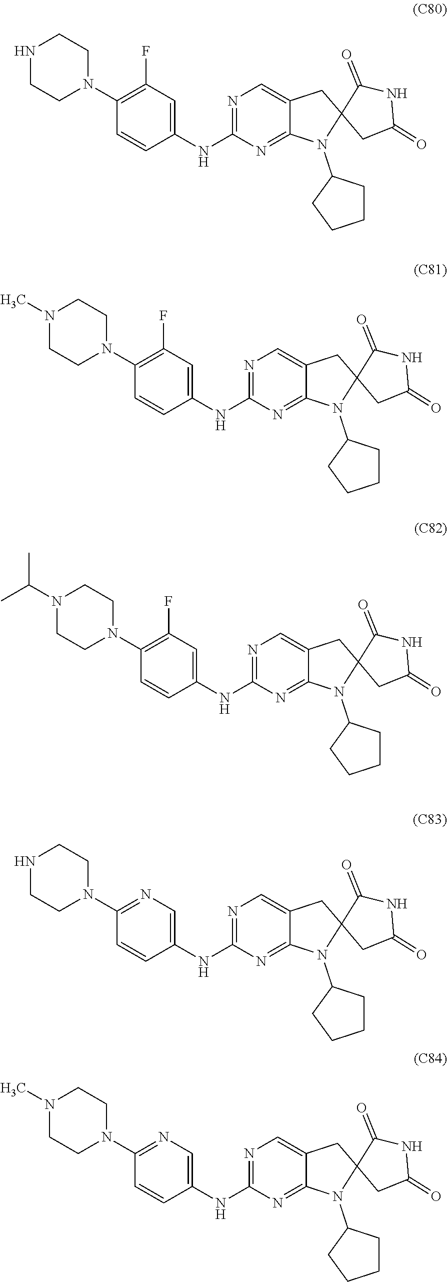 [SMILES: O=C1CC2(Cc3cnc(Nc4ccc(N5CCNCC5)c(F)c4)nc3N2C2CCCC2)C(=O)N1 ; CN1CCN(c2ccc(Nc3ncc4c(n3)N(C3CCCC3)C3(CC(=O)NC3=O)C4)cc2F)CC1 ; CC(C)N1CCN(c2ccc(Nc3ncc4c(n3)N(C3CCCC3)C3(CC(=O)NC3=O)C4)cc2F)CC1 ; O=C1CC2(Cc3cnc(Nc4ccc(N5CCNCC5)nc4)nc3N2C2CCCC2)C(=O)N1 ; CN1CCN(c2ccc(Nc3ncc4c(n3)N(C3CCCC3)C3(CC(=O)NC3=O)C4)cn2)CC1]

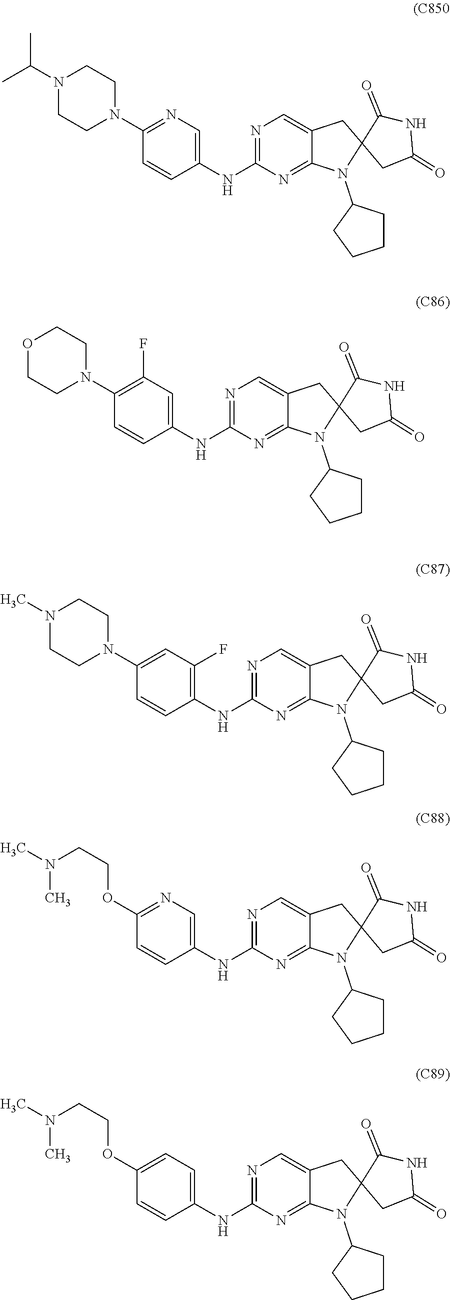 [SMILES: CC(C)N1CCN(c2ccc(Nc3ncc4c(n3)N(C3CCCC3)C3(CC(=O)NC3=O)C4)cn2)CC1 ; O=C1CC2(Cc3cnc(Nc4ccc(N5CCOCC5)c(F)c4)nc3N2C2CCCC2)C(=O)N1 ; CN1CCN(c2ccc(Nc3ncc4c(n3)N(C3CCCC3)C3(CC(=O)NC3=O)C4)c(F)c2)CC1 ; CN(C)CCOc1ccc(Nc2ncc3c(n2)N(C2CCCC2)C2(CC(=O)NC2=O)C3)cn1 ; CN(C)CCOc1ccc(Nc2ncc3c(n2)N(C2CCCC2)C2(CC(=O)NC2=O)C3)cc1]

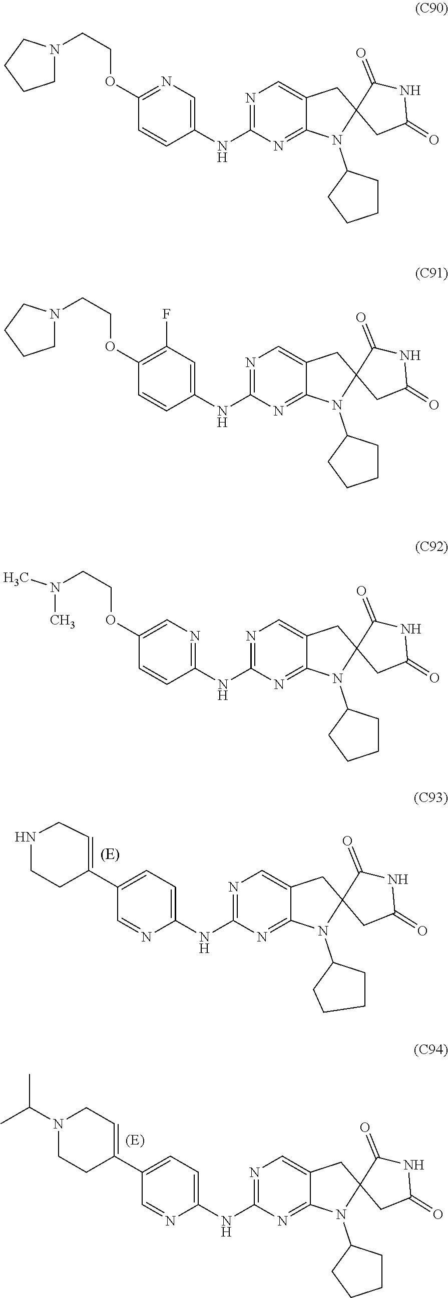 [SMILES: O=C1CC2(Cc3cnc(Nc4ccc(OCCN5CCCC5)nc4)nc3N2C2CCCC2)C(=O)N1 ; O=C1CC2(Cc3cnc(Nc4ccc(OCCN5CCCC5)c(F)c4)nc3N2C2CCCC2)C(=O)N1 ; CN(C)CCOc1ccc(Nc2ncc3c(n2)N(C2CCCC2)C2(CC(=O)NC2=O)C3)nc1 ; O=C1CC2(Cc3cnc(Nc4ccc(C5=CCNCC5)cn4)nc3N2C2CCCC2)C(=O)N1 ; CC(C)N1CC=C(c2ccc(Nc3ncc4c(n3)N(C3CCCC3)C3(CC(=O)NC3=O)C4)nc2)CC1]

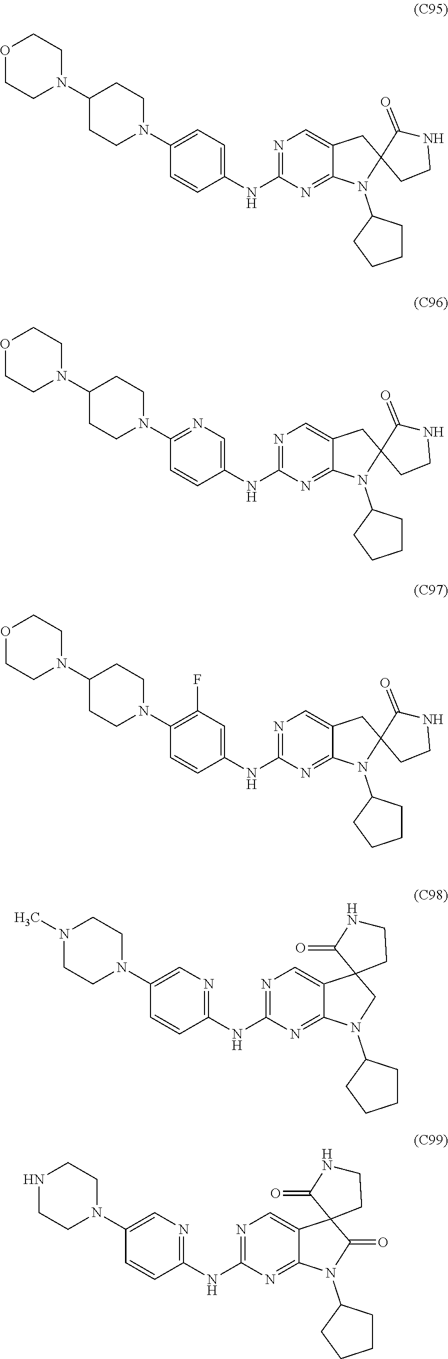 [SMILES: O=C1NCCC12Cc1cnc(Nc3ccc(N4CCC(N5CCOCC5)CC4)cc3)nc1N2C1CCCC1 ; O=C1NCCC12Cc1cnc(Nc3ccc(N4CCC(N5CCOCC5)CC4)nc3)nc1N2C1CCCC1 ; O=C1NCCC12Cc1cnc(Nc3ccc(N4CCC(N5CCOCC5)CC4)c(F)c3)nc1N2C1CCCC1 ; CN1CCN(c2ccc(Nc3ncc4c(n3)N(C3CCCC3)CC43CCNC3=O)nc2)CC1 ; O=C1NCCC12C(=O)N(C1CCCC1)c1nc(Nc3ccc(N4CCNCC4)cn3)ncc12]

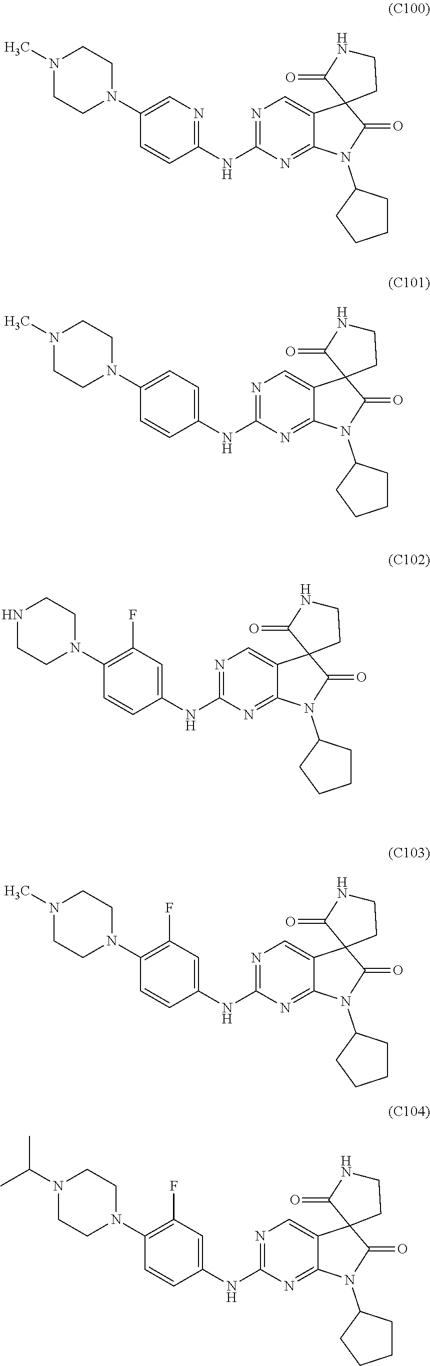 [SMILES: CN1CCN(c2ccc(Nc3ncc4c(n3)N(C3CCCC3)C(=O)C43CCNC3=O)nc2)CC1 ; CN1CCN(c2ccc(Nc3ncc4c(n3)N(C3CCCC3)C(=O)C43CCNC3=O)cc2)CC1 ; O=C1NCCC12C(=O)N(C1CCCC1)c1nc(Nc3ccc(N4CCNCC4)c(F)c3)ncc12 ; CN1CCN(c2ccc(Nc3ncc4c(n3)N(C3CCCC3)C(=O)C43CCNC3=O)cc2F)CC1 ; CC(C)N1CCN(c2ccc(Nc3ncc4c(n3)N(C3CCCC3)C(=O)C43CCNC3=O)cc2F)CC1]

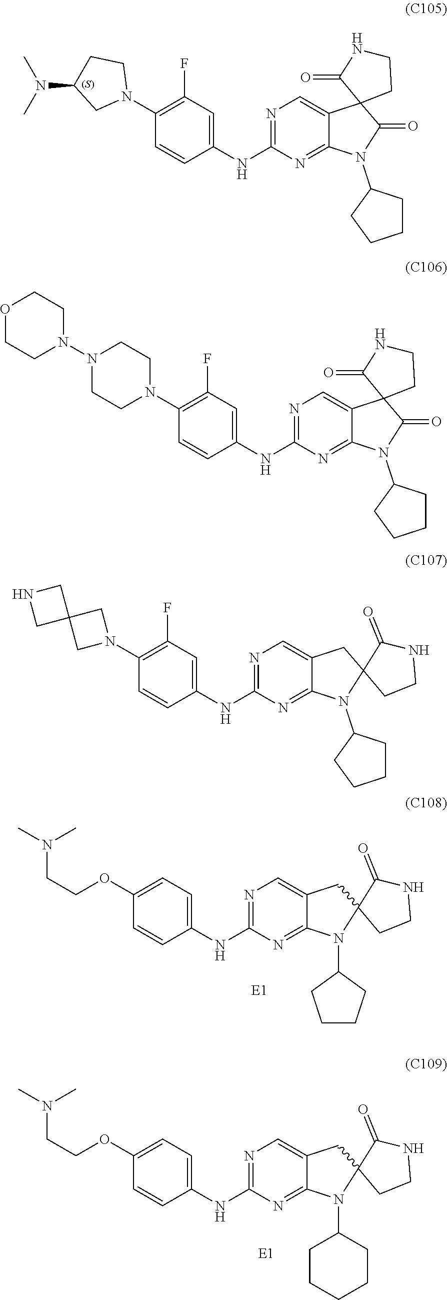

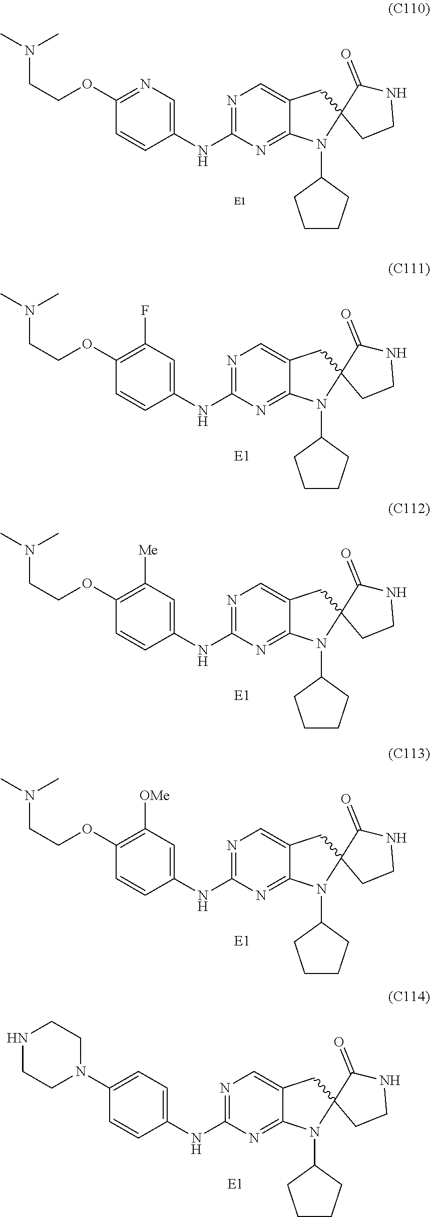 [SMILES: CN(C)CCOc1ccc(Nc2ncc3c(n2)N(C2CCCC2)C2(CCNC2=O)C3)cn1 ; CN(C)CCOc1ccc(Nc2ncc3c(n2)N(C2CCCC2)C2(CCNC2=O)C3)cc1F ; Cc1cc(Nc2ncc3c(n2)N(C2CCCC2)C2(CCNC2=O)C3)ccc1OCCN(C)C ; COc1cc(Nc2ncc3c(n2)N(C2CCCC2)C2(CCNC2=O)C3)ccc1OCCN(C)C ; O=C1NCCC12Cc1cnc(Nc3ccc(N4CCNCC4)cc3)nc1N2C1CCCC1]

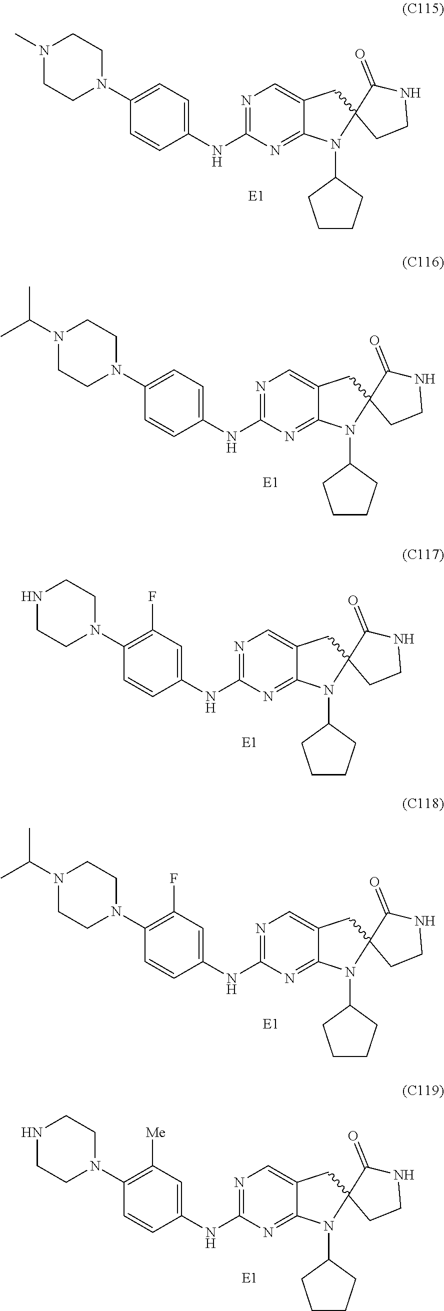 [SMILES: CN1CCN(c2ccc(Nc3ncc4c(n3)N(C3CCCC3)C3(CCNC3=O)C4)cc2)CC1 ; CC(C)N1CCN(c2ccc(Nc3ncc4c(n3)N(C3CCCC3)C3(CCNC3=O)C4)cc2)CC1 ; O=C1NCCC12Cc1cnc(Nc3ccc(N4CCNCC4)c(F)c3)nc1N2C1CCCC1 ; CC(C)N1CCN(c2ccc(Nc3ncc4c(n3)N(C3CCCC3)C3(CCNC3=O)C4)cc2F)CC1 ; Cc1cc(Nc2ncc3c(n2)N(C2CCCC2)C2(CCNC2=O)C3)ccc1N1CCNCC1]

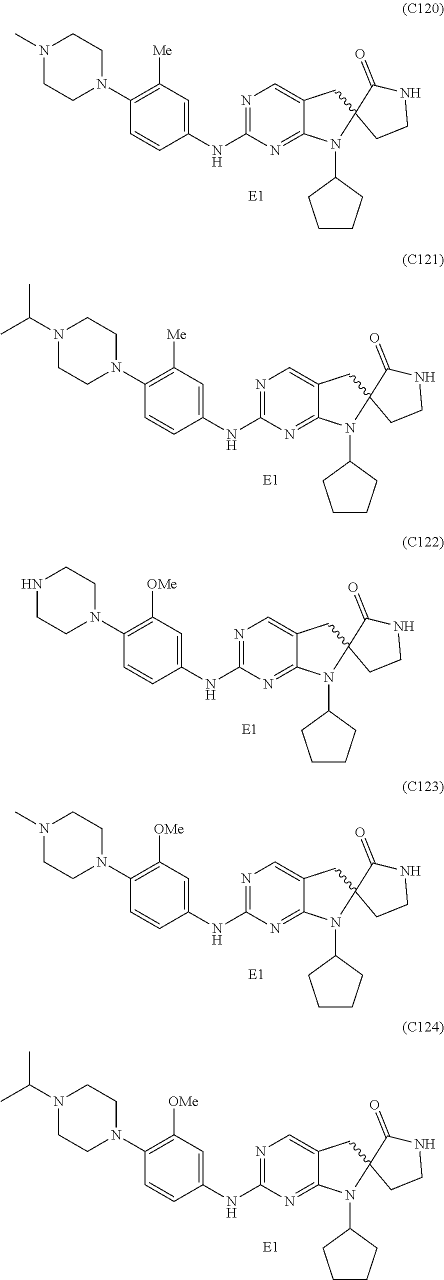 [SMILES: Cc1cc(Nc2ncc3c(n2)N(C2CCCC2)C2(CCNC2=O)C3)ccc1N1CCN(C)CC1 ; Cc1cc(Nc2ncc3c(n2)N(C2CCCC2)C2(CCNC2=O)C3)ccc1N1CCN(C(C)C)CC1 ; COc1cc(Nc2ncc3c(n2)N(C2CCCC2)C2(CCNC2=O)C3)ccc1N1CCNCC1 ; COc1cc(Nc2ncc3c(n2)N(C2CCCC2)C2(CCNC2=O)C3)ccc1N1CCN(C)CC1 ; COc1cc(Nc2ncc3c(n2)N(C2CCCC2)C2(CCNC2=O)C3)ccc1N1CCN(C(C)C)CC1]

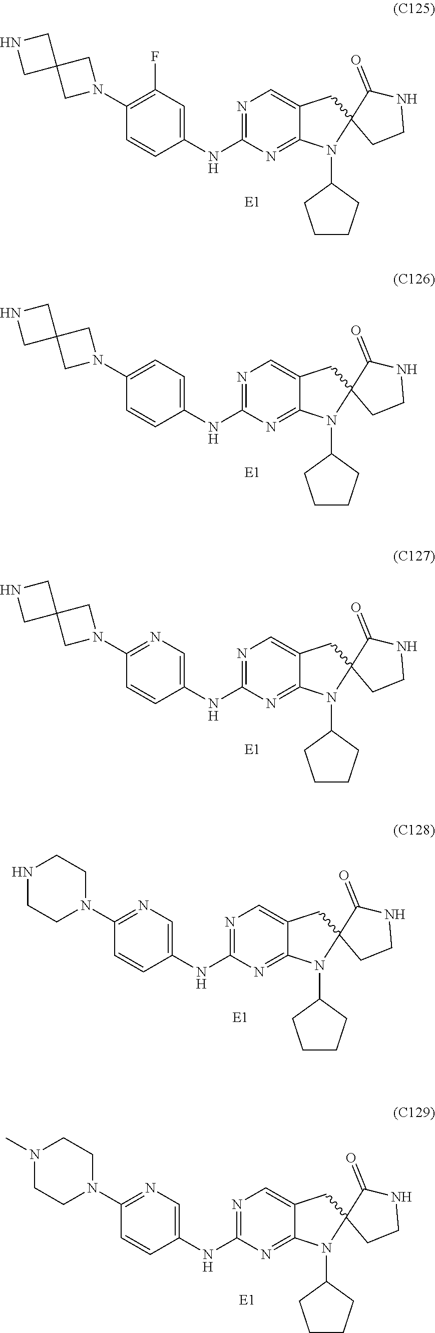 [SMILES: O=C1NCCC12Cc1cnc(Nc3ccc(N4CC5(CNC5)C4)c(F)c3)nc1N2C1CCCC1 ; O=C1NCCC12Cc1cnc(Nc3ccc(N4CC5(CNC5)C4)cc3)nc1N2C1CCCC1 ; O=C1NCCC12Cc1cnc(Nc3ccc(N4CC5(CNC5)C4)nc3)nc1N2C1CCCC1 ; O=C1NCCC12Cc1cnc(Nc3ccc(N4CCNCC4)nc3)nc1N2C1CCCC1 ; CN1CCN(c2ccc(Nc3ncc4c(n3)N(C3CCCC3)C3(CCNC3=O)C4)cn2)CC1]

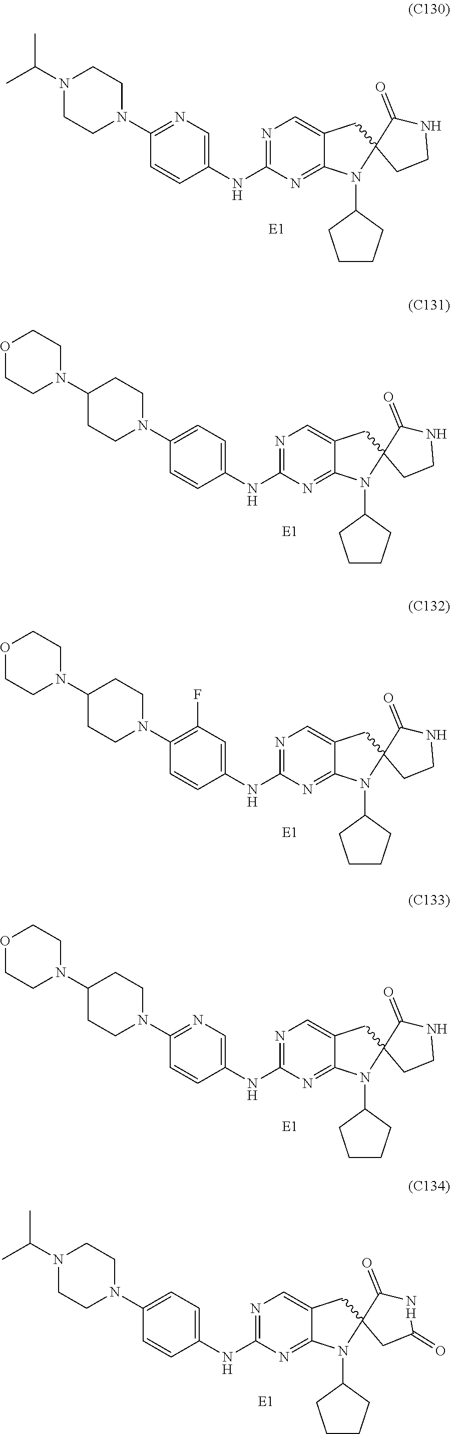 [SMILES: CC(C)N1CCN(c2ccc(Nc3ncc4c(n3)N(C3CCCC3)C3(CCNC3=O)C4)cn2)CC1 ; O=C1NCCC12Cc1cnc(Nc3ccc(N4CCC(N5CCOCC5)CC4)cc3)nc1N2C1CCCC1 ; O=C1NCCC12Cc1cnc(Nc3ccc(N4CCC(N5CCOCC5)CC4)c(F)c3)nc1N2C1CCCC1 ; O=C1NCCC12Cc1cnc(Nc3ccc(N4CCC(N5CCOCC5)CC4)nc3)nc1N2C1CCCC1 ; CC(C)N1CCN(c2ccc(Nc3ncc4c(n3)N(C3CCCC3)C3(CC(=O)NC3=O)C4)cc2)CC1]

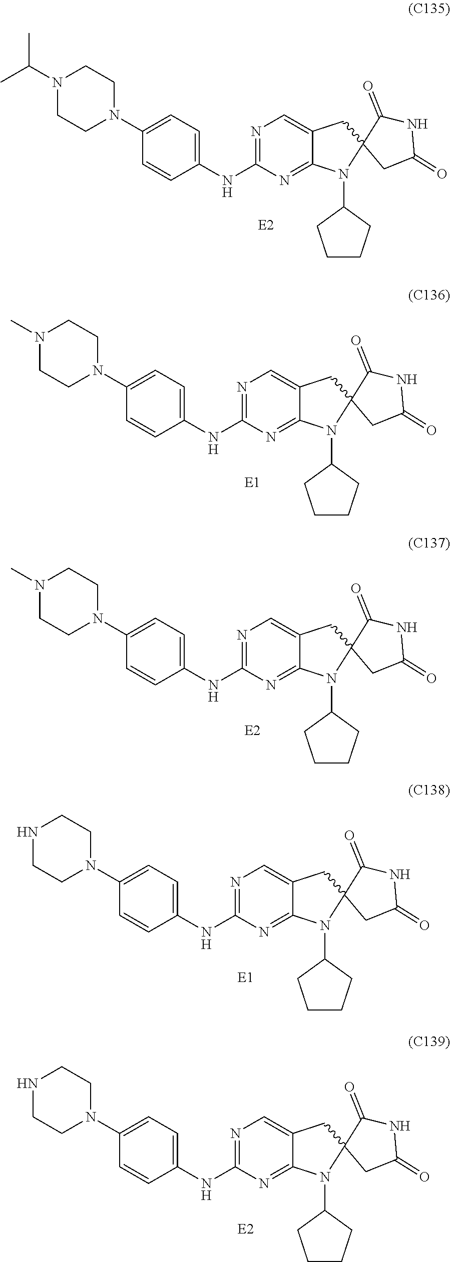 [SMILES: CC(C)N1CCN(c2ccc(Nc3ncc4c(n3)N(C3CCCC3)C3(CC(=O)NC3=O)C4)cc2)CC1 ; CN1CCN(c2ccc(Nc3ncc4c(n3)N(C3CCCC3)C3(CC(=O)NC3=O)C4)cc2)CC1 ; CN1CCN(c2ccc(Nc3ncc4c(n3)N(C3CCCC3)C3(CC(=O)NC3=O)C4)cc2)CC1 ; O=C1CC2(Cc3cnc(Nc4ccc(N5CCNCC5)cc4)nc3N2C2CCCC2)C(=O)N1 ; O=C1CC2(Cc3cnc(Nc4ccc(N5CCNCC5)cc4)nc3N2C2CCCC2)C(=O)N1]

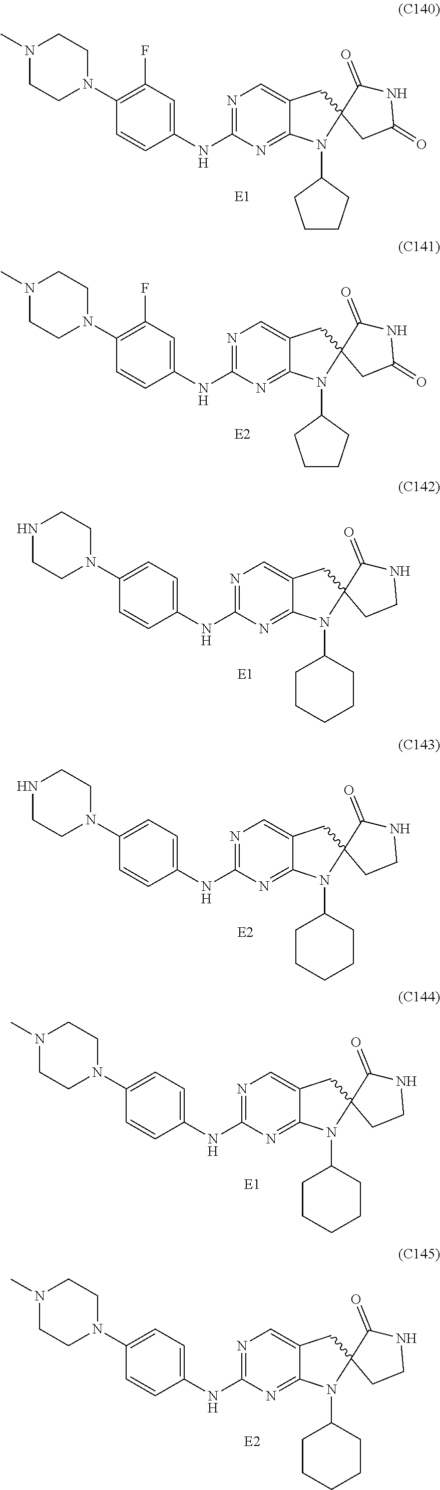 [SMILES: CN1CCN(c2ccc(Nc3ncc4c(n3)N(C3CCCC3)C3(CC(=O)NC3=O)C4)cc2F)CC1 ; CN1CCN(c2ccc(Nc3ncc4c(n3)N(C3CCCC3)C3(CC(=O)NC3=O)C4)cc2F)CC1 ; O=C1NCCC12Cc1cnc(Nc3ccc(N4CCNCC4)cc3)nc1N2C1CCCCC1 ; O=C1NCCC12Cc1cnc(Nc3ccc(N4CCNCC4)cc3)nc1N2C1CCCCC1 ; CN1CCN(c2ccc(Nc3ncc4c(n3)N(C3CCCCC3)C3(CCNC3=O)C4)cc2)CC1 ; CN1CCN(c2ccc(Nc3ncc4c(n3)N(C3CCCCC3)C3(CCNC3=O)C4)cc2)CC1]

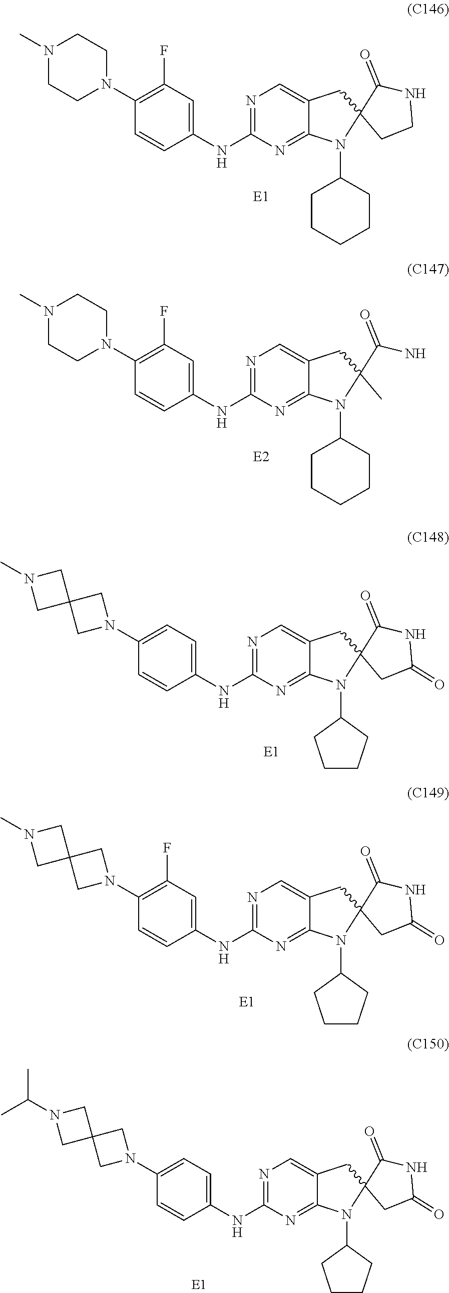 [SMILES: CN1CCN(c2ccc(Nc3ncc4c(n3)N(C3CCCCC3)C3(CCNC3=O)C4)cc2F)CC1 ; CN1CCN(c2ccc(Nc3ncc4c(n3)N(C3CCCCC3)C(C)(C(N)=O)C4)cc2F)CC1 ; CN1CC2(C1)CN(c1ccc(Nc3ncc4c(n3)N(C3CCCC3)C3(CC(=O)NC3=O)C4)cc1)C2 ; CN1CC2(C1)CN(c1ccc(Nc3ncc4c(n3)N(C3CCCC3)C3(CC(=O)NC3=O)C4)cc1F)C2 ; CC(C)N1CC2(CN(c3ccc(Nc4ncc5c(n4)N(C4CCCC4)C4(CC(=O)NC4=O)C5)cc3)C2)C1]

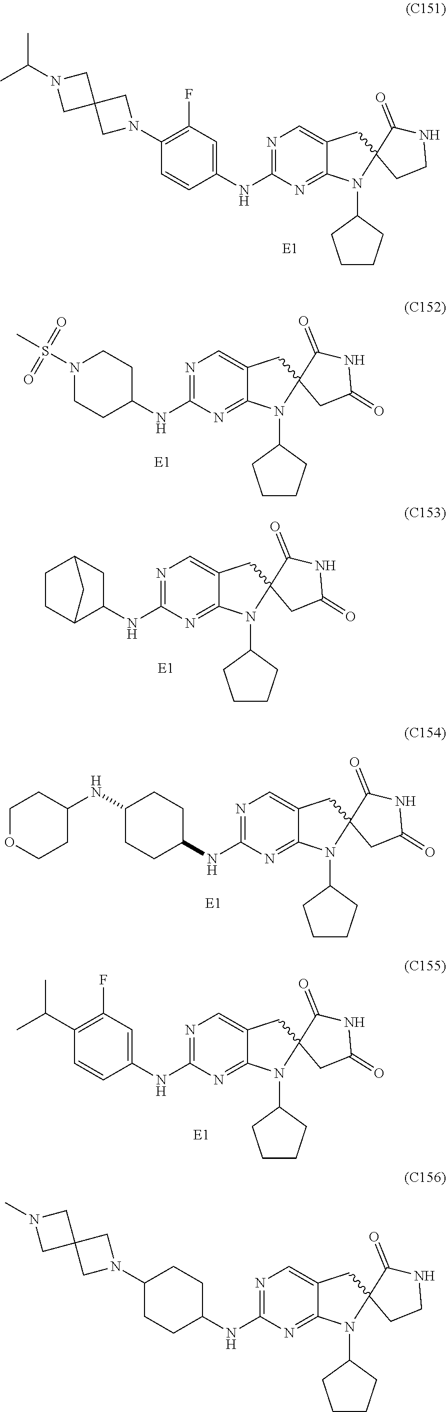

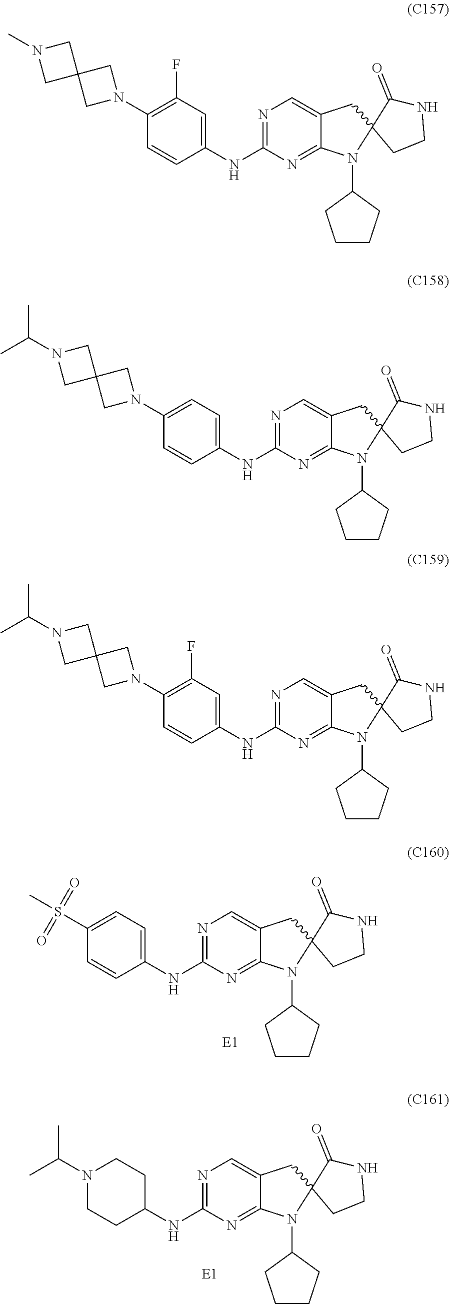 [SMILES: CN1CC2(C1)CN(c1ccc(Nc3ncc4c(n3)N(C3CCCC3)C3(CCNC3=O)C4)cc1F)C2 ; CC(C)N1CC2(CN(c3ccc(Nc4ncc5c(n4)N(C4CCCC4)C4(CCNC4=O)C5)cc3)C2)C1 ; CC(C)N1CC2(CN(c3ccc(Nc4ncc5c(n4)N(C4CCCC4)C4(CCNC4=O)C5)cc3F)C2)C1 ; CS(=O)(=O)c1ccc(Nc2ncc3c(n2)N(C2CCCC2)C2(CCNC2=O)C3)cc1 ; CC(C)N1CCC(Nc2ncc3c(n2)N(C2CCCC2)C2(CCNC2=O)C3)CC1]

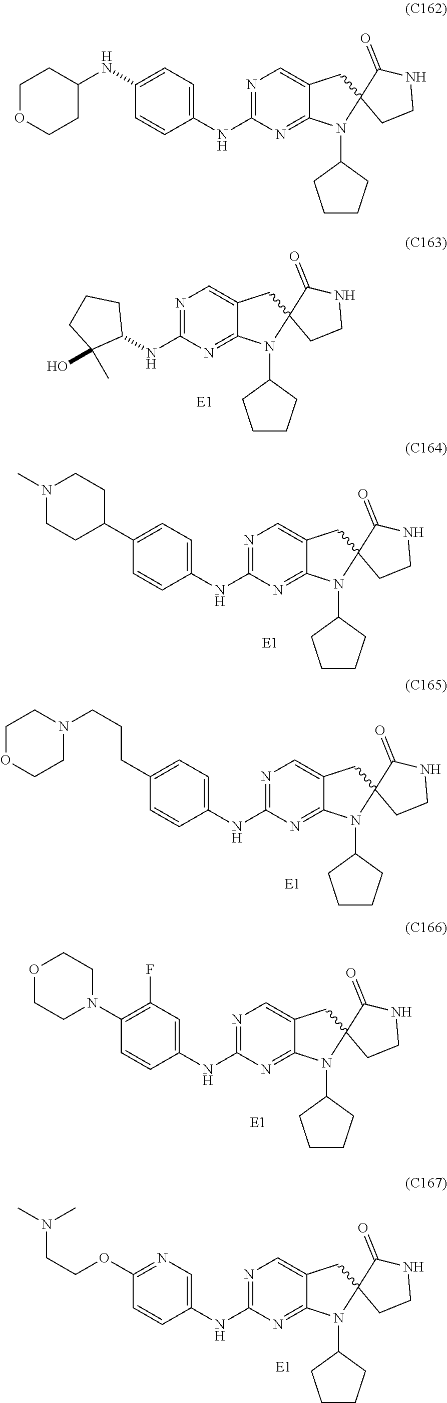

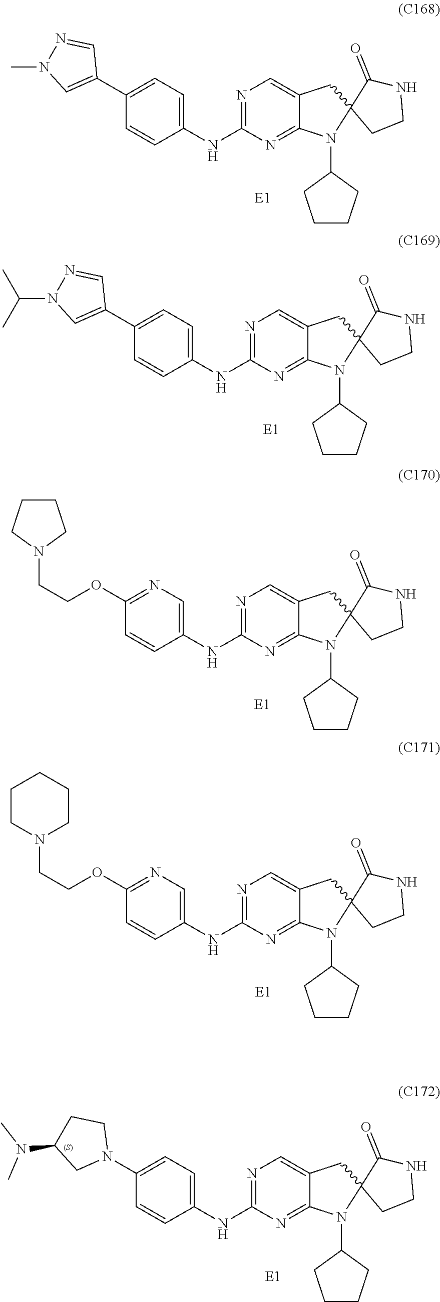

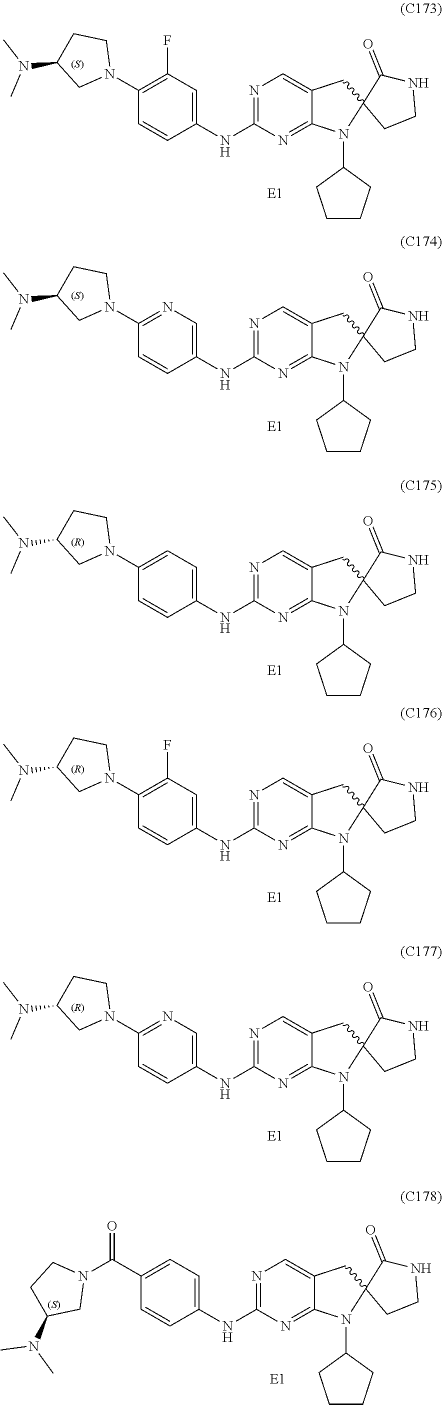

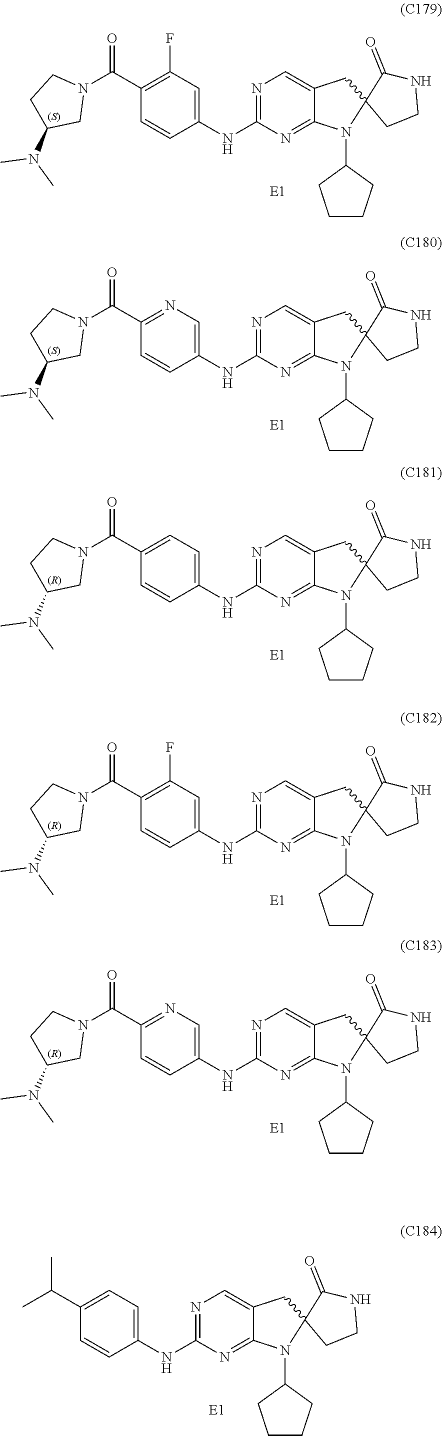

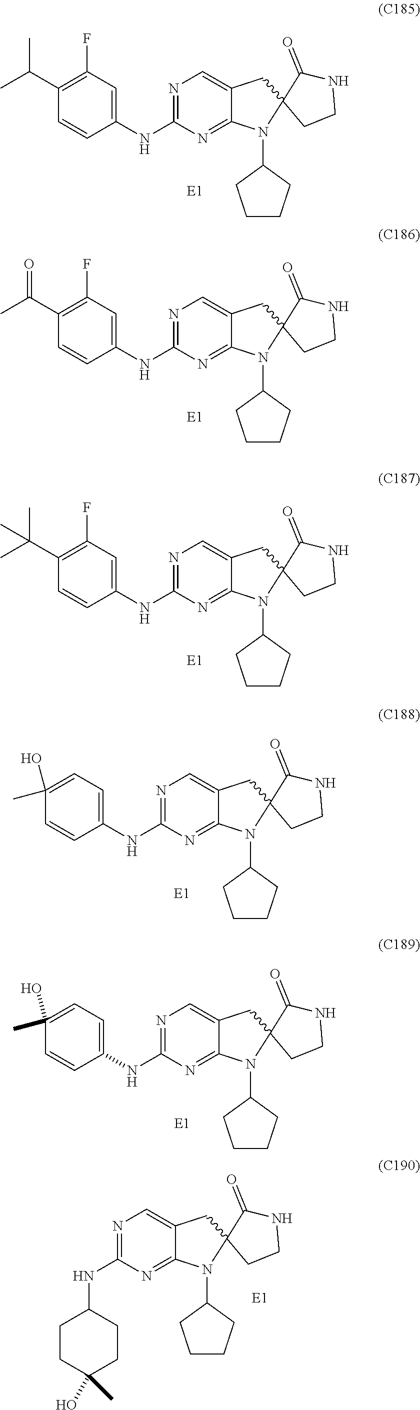

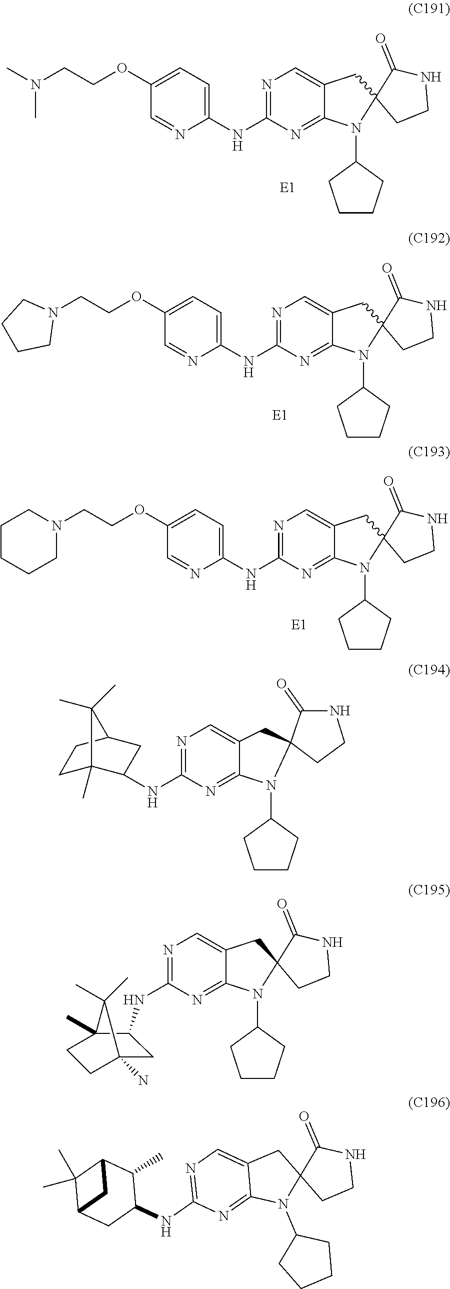

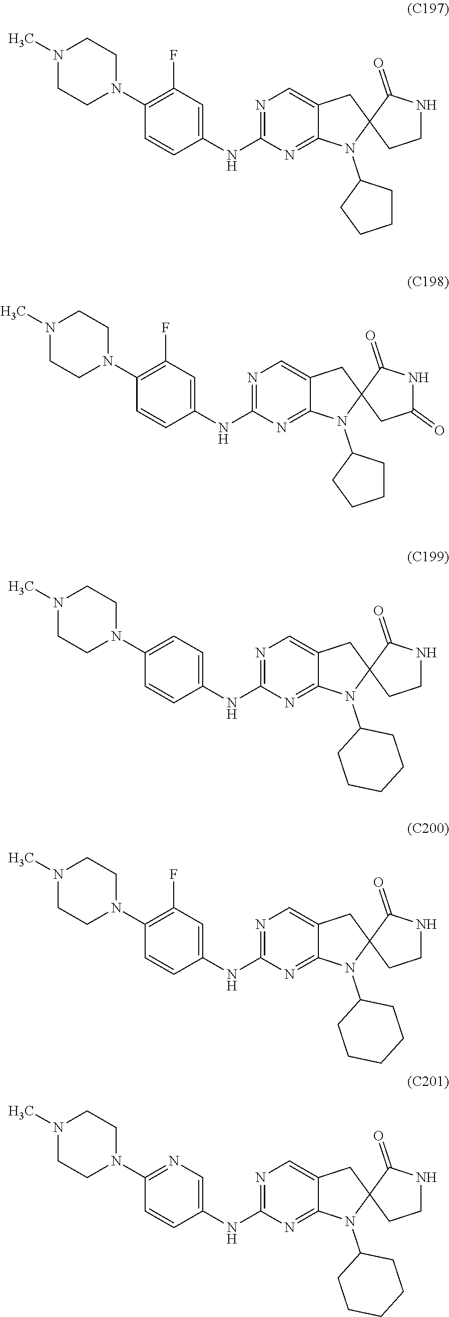 [SMILES: CN1CCN(c2ccc(Nc3ncc4c(n3)N(C3CCCC3)C3(CCNC3=O)C4)cc2F)CC1 ; CN1CCN(c2ccc(Nc3ncc4c(n3)N(C3CCCC3)C3(CC(=O)NC3=O)C4)cc2F)CC1 ; CN1CCN(c2ccc(Nc3ncc4c(n3)N(C3CCCCC3)C3(CCNC3=O)C4)cc2)CC1 ; CN1CCN(c2ccc(Nc3ncc4c(n3)N(C3CCCCC3)C3(CCNC3=O)C4)cc2F)CC1 ; CN1CCN(c2ccc(Nc3ncc4c(n3)N(C3CCCCC3)C3(CCNC3=O)C4)cn2)CC1]

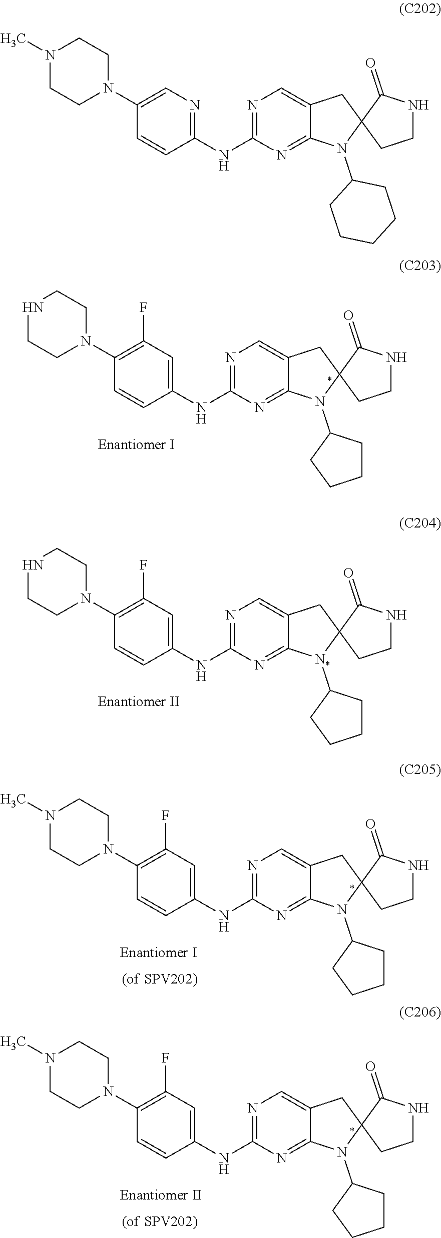 [SMILES: CN1CCN(c2ccc(Nc3ncc4c(n3)N(C3CCCCC3)C3(CCNC3=O)C4)nc2)CC1 ; O=C1NCCC12Cc1cnc(Nc3ccc(N4CCNCC4)c(F)c3)nc1N2C1CCCC1 ; O=C1NCCC12Cc1cnc(Nc3ccc(N4CCNCC4)c(F)c3)nc1N2C1CCCC1 ; CN1CCN(c2ccc(Nc3ncc4c(n3)N(C3CCCC3)C3(CCNC3=O)C4)cc2F)CC1 ; CN1CCN(c2ccc(Nc3ncc4c(n3)N(C3CCCC3)C3(CCNC3=O)C4)cc2F)CC1]

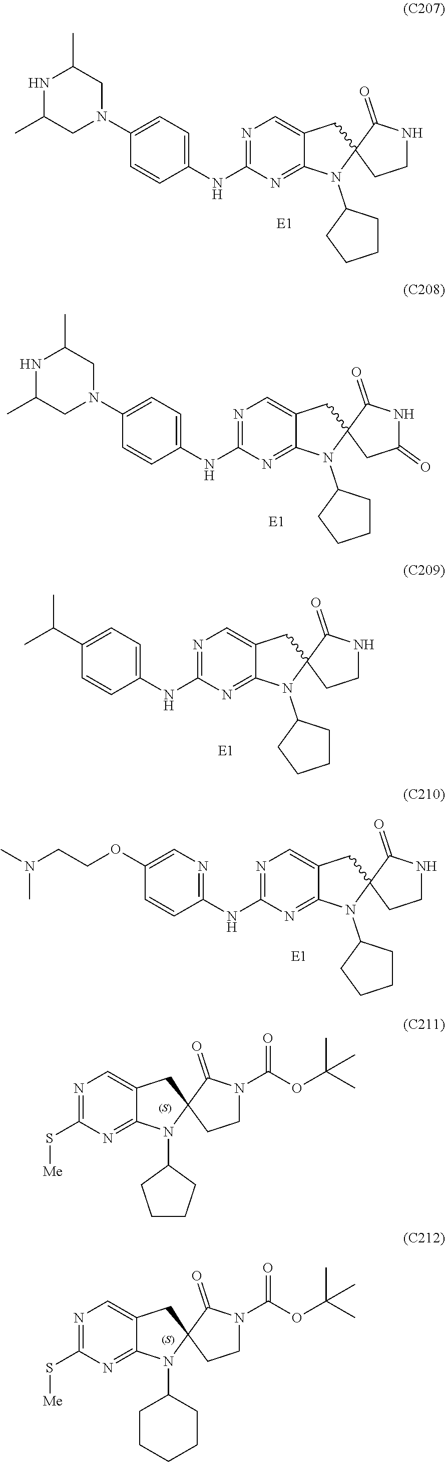

21. A pharmaceutical composition comprising one or more of Compounds 01-0212 or a pharmaceutically acceptable salt thereof and one or more pharmaceutically acceptable excipients.

22. A method of treating cancer or providing a chemotherapeutic protective effect comprising administering an effective amount of one or more of Compounds 01-0212 to a subject in need thereof.

23. A compound selected from:
7′-cyclopentyl-1-methyl-2′-((4-(4-methylpiperazin-1-yl)phenyl)amino)-5′,7′-dihydrospiro[pyrrolidine-3,6′-pyrrolo[2,3-d]pyrimidin]-2-one (C1);
7′-cyclopentyl-1-methyl-2′-((4-(piperazin-1-yl)phenyl)amino)-5′,7′-dihydrospiro[pyrrolidine-3,6′-pyrrolo[2,3-d]pyrimidin]-2-one (C2);
7′-cyclopentyl-2′-((5-(4-isopropylpiperazin-1-yl)pyridin-2-yl)amino)-1-methyl-5′,7′-dihydrospiro[pyrrolidine-3,6′-pyrrolo[2,3-d]pyrimidin]-2-one (C3);
7′-cyclopentyl-2′-((5-(2-(dimethylamino)ethoxy)pyridin-2-yl)amino)-1-methyl-5′,7′-dihydrospiro[pyrrolidine-3,6′-pyrrolo[2,3-d]pyrimidin]-2-one (C4);
7′-cyclopentyl-1-methyl-2′-((5-(2-(pyrrolidin-1-yl)ethoxy)pyridin-2-yl)amino)-5′,7′-dihydrospiro[pyrrolidine-3,6′-pyrrolo[2,3-d]pyrimidin]-2-one (C5);
7′-cyclopentyl-1-methyl-2′-((5-(2-(piperidin-1-yl)ethoxy)pyridin-2-yl)amino)-5′,7′-dihydrospiro[pyrrolidine-3,6′-pyrrolo[2,3-d]pyrimidin]-2-one (C6);
7′-cyclopentyl-1-methyl-2′-((4-(4-methylpiperazin-1-yl)phenyl)amino)-5′,7′-dihydrospiro[piperidine-3,6′-pyrrolo[2,3-d]pyrimidin]-2-one (C7);
7′-cyclopentyl-1-methyl-2′-((4-(piperazin-1-yl)phenyl)amino)-5′,7′-dihydrospiro[piperidine-3,6′-pyrrolo[2,3-d]pyrimidin]-2-one (C8);
7′-cyclopentyl-2′-((5-(4-isopropylpiperazin-1-yl)pyridin-2-yl)amino)-1-methyl-5′,7′-dihydrospiro[piperidine-3,6′-pyrrolo[2,3-d]pyrimidin]-2-one (C9);
7′-cyclopentyl-1-methyl-2′-((5-(2-(pyrrolidin-1-yl)ethoxy)pyridin-2-yl)amino)-5′,7′-dihydrospiro[piperidine-3,6′-pyrrolo[2,3-d]pyrimidin]-2-one (C10);
7′-cyclopentyl-1-methyl-2′-((5-(2-(piperidin-1-yl)ethoxy)pyridin-2-yl)amino)-5′,7′-dihydrospiro[piperidine-3,6′-pyrrolo[2,3-d]pyrimidin]-2-one (C11);
7′-cyclopentyl-1-(4-methoxybenzyl)-2′-((4-(4-methylpiperazin-1-yl)phenyl)amino)-5′,7′-dihydrospiro[pyrrolidine-3,6′-pyrrolo[2,3-d]pyrimidin]-2-one (C12);
7′-cyclopentyl-1-methyl-2′-((5-(4-methylpiperazin-1-yl)-4-(trifluoromethyl)pyridin-2-yl)amino)-5′,7′-dihydrospiro[pyrrolidine-3,6′-pyrrolo[2,3-d]pyrimidin]-2-one (C13);
7′-cyclopentyl-1-methyl-2′-((5-(piperazin-1-yl)-4-(trifluoromethyl)pyridin-2-yl)amino)-5′,7′-dihydrospiro[piperidine-3,6′-pyrrolo[2,3-d]pyrimidin]-2-one (C14);
7′-cyclopentyl-1-methyl-2′-((5-(piperazin-1-yl)-4-(trifluoromethyl)pyridin-2-yl)amino)-5′,7′-dihydrospiro[pyrrolidine-3,6′-pyrrolo[2,3-d]pyrimidin]-2-one (C15);
7′-cyclopentyl-1-(4-methoxybenzyl)-2′-((5-(piperazin-1-yl)-4-(trifluoromethyl)pyridin-2-yl)amino)-5′,7′-dihydrospiro[pyrrolidine-3,6′-pyrrolo[2,3-d]pyrimidin]-2-one (C16);
7′-cyclopentyl-2′-((5-(piperazin-1-yl)-4-(trifluoromethyl)pyridin-2-yl)amino)-5′,7′-dihydrospiro[pyrrolidine-3,6′-pyrrolo[2,3-d]pyrimidin]-2-one (C17);
7′-cyclopentyl-2′-((5-(4-methylpiperazin-1-yl)-4-(trifluoromethyl)pyridin-2-yl)amino)-5′,7′-dihydrospiro[pyrrolidine-3,6′-pyrrolo[2,3-d]pyrimidin]-2-one (C18);
7′-cyclopentyl-1-methyl-2′-((5-(piperazin-1-yl)pyridin-2-yl)amino)-5′,7′-dihydrospiro[pyrrolidine-3,6′-pyrrolo[2,3-d]pyrimidin]-2-one (C19);
7′-cyclopentyl-1-methyl-2′-((5-(4-methylpiperazin-1-yl)pyridin-2-yl)amino)-5′,7′-dihydrospiro[pyrrolidine-3,6′-pyrrolo[2,3-d]pyrimidin]-2-one (C20);
1-benzyl-7′-cyclopentyl-2′-((5-(piperazin-1-yl)pyridin-2-yl)amino)-5′,7′-dihydrospiro[pyrrolidine-3,6′-pyrrolo[2,3-d]pyrimidin]-2-one (C21);
1-benzyl-7′-cyclopentyl-2′-((5-(4-methylpiperazin-1-yl)pyridin-2-yl)amino)-5′,7′-dihydrospiro[pyrrolidine-3,6′-pyrrolo[2,3-d]pyrimidin]-2-one (C22);
1-benzyl-7′-cyclopentyl-2′-((5-(4-isopropylpiperazin-1-yl)pyridin-2-yl)amino)-5′,7′-dihydrospiro[pyrrolidine-3,6′-pyrrolo[2,3-d]pyrimidin]-2-one (C23);
1-benzyl-7′-cyclopentyl-2′-((4-(4-methylpiperazin-1-yl)phenyl)amino)-5′,7′-dihydrospiro[pyrrolidine-3,6′-pyrrolo[2,3-d]pyrimidin]-2-one (C24);
7′-cyclopentyl-1-methyl-2′-((5-(piperazin-1-yl)pyridin-2-yl)amino)-5′,7′-dihydrospiro[piperidine-3,6′-pyrrolo[2,3-d]pyrimidin]-2-one (C25);
7′-cyclopentyl-1-methyl-2′-((5-(4-methylpiperazin-1-yl)pyridin-2-yl)amino)-5′,7′-dihydrospiro[piperidine-3,6′-pyrrolo[2,3-d]pyrimidin]-2-one (C26);
1-benzyl-7′-cyclopentyl-2′-((5-(piperazin-1-yl)pyridin-2-yl)amino)-5′,7′-dihydrospiro[piperidine-3,6′-pyrrolo[2,3-d]pyrimidin]-2-one (C27);
1-benzyl-7′-cyclopentyl-2′-((5-(4-methylpiperazin-1-yl)pyridin-2-yl)amino)-5′,7′-dihydrospiro[piperidine-3,6′-pyrrolo[2,3-d]pyrimidin]-2-one (C28);
7′-cyclopentyl-1-(4-methoxybenzyl)-2′-((5-(piperazin-1-yl)pyridin-2-yl)amino)-5′,7′-dihydrospiro[pyrrolidine-3,6′-pyrrolo[2,3-d]pyrimidin]-2-one (C29);
7′-cyclopentyl-1-(4-methoxybenzyl)-2′-((5-(4-methylpiperazin-1-yl)pyridin-2-yl)amino)-5′,7′-dihydrospiro[pyrrolidine-3,6′-pyrrolo[2,3-d]pyrimidin]-2-one (C30);
7′-cyclopentyl-2′-((5-(4-isopropylpiperazin-1-yl)pyridin-2-yl)amino)-1-(4-methoxybenzyl)-5′,7′-dihydrospiro[pyrrolidine-3,6′-pyrrolo[2,3-d]pyrimidin]-2-one (C31);
7′-cyclopentyl-2′-((5-(2-(dimethylamino)ethoxy)pyridin-2-yl)amino)-1-(4-methoxybenzyl)-5′,7′-dihydrospiro[pyrrolidine-3,6′-pyrrolo[2,3-d]pyrimidin]-2-one (C32);
7′-cyclopentyl-2′-((5-(piperazin-1-yl)pyridin-2-yl)amino)-5′,7′-dihydrospiro[pyrrolidine-3,6′-pyrrolo[2,3-d]pyrimidin]-2-one (C33);
7′-cyclopentyl-2′-((5-(4-methylpiperazin-1-yl)pyridin-2-yl)amino)-5′,7′-dihydrospiro[pyrrolidine-3,6′-pyrrolo[2,3-d]pyrimidin]-2-one (C34);
7′-cyclopentyl-2′-((5-(piperazin-1-yl)pyridin-2-yl)amino)-5′,7′-dihydrospiro[piperidine-3,6′-pyrrolo[2,3-d]pyrimidin]-2-one (C35);
7′-cyclopentyl-2′-((5-(4-methylpiperazin-1-yl)pyridin-2-yl)amino)-5′,7′-dihydrospiro[piperidine-3,6′-pyrrolo[2,3-d]pyrimidin]-2-one (C36);
7′-cyclopentyl-2′-((5-(4-isopropylpiperazin-1-yl)pyridin-2-yl)amino)-5′,7′-dihydrospiro[piperidine-3,6′-pyrrolo[2,3-d]pyrimidin]-2-one (C37);
7′-cyclopentyl-2′-((5-(4-isopropylpiperazin-1-yl)pyridin-2-yl)amino)-5′,7′-dihydrospiro[pyrrolidine-3,6′-pyrrolo[2,3-d]pyrimidin]-2-one (C38);
1-benzyl-7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one (C39);
1-benzyl-7′-cyclopentyl-2′-((3-fluoro-4-(4-methylpiperazin-1-yl)phenyl)amino)-5′,7′-dihydrospiro[piperidine-3,6′-pyrrolo[2,3-d]pyrimidin]-2-one (C40);
1-benzyl-7′-cyclopentyl-2′-((2-fluoro-4-(4-methylpiperazin-1-yl)phenyl)amino)-5′,7′-dihydrospiro[pyrrolidine-3,6′-pyrrolo[2,3-d]pyrimidin]-2-one (C41);
1-benzyl-7′-cyclopentyl-2′-((2-fluoro-4-(4-methylpiperazin-1-yl)phenyl)amino)-5′,7′-dihydrospiro[piperidine-3,6′-pyrrolo[2,3-d]pyrimidin]-2-one (C42);
7′-cyclopentyl-2′-((4-(piperazin-1-yl)phenyl)amino)-5′,7′-dihydrospiro[pyrrolidine-3,6′-pyrrolo[2,3-d]pyrimidin]-2-one (C43);
7′-cyclopentyl-2′-((5-(piperazin-1-yl)-4-(trifluoromethyl)pyridin-2-yl)amino)-5′,7′-dihydrospiro[piperidine-3,6′-pyrrolo[2,3-d]pyrimidin]-2-one (C44);
7′-cyclopentyl-2′-((4-(4-methylpiperazin-1-yl)phenyl)amino)-5′,7′-dihydrospiro[pyrrolidine-3,6′-pyrrolo[2,3-d]pyrimidin]-2-one (C45);
7′-cyclopentyl-2′-((4-(4-isopropylpiperazin-1-yl)phenyl)amino)-5′,7′-dihydrospiro[pyrrolidine-3,6′-pyrrolo[2,3-d]pyrimidin]-2-one (C46);
7′-cyclopentyl-2′-((3-fluoro-4-(piperazin-1-yl)phenyl)amino)-5′,7′-dihydrospiro[pyrrolidine-3,6′-pyrrolo[2,3-d]pyrimidin]-2-one (C47);
7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one (C48);
7′-cyclopentyl-2′-((3-fluoro-4-(4-isopropylpiperazin-1-yl)phenyl)amino)-5′,7′-dihydrospiro[pyrrolidine-3,6′-pyrrolo[2,3-d]pyrimidin]-2-one (C49);
7′-cyclopentyl-2′-((2-fluoro-4-(piperazin-1-yl)phenyl)amino)-5′,7′-dihydrospiro[pyrrolidine-3,6′-pyrrolo[2,3-d]pyrimidin]-2-one (C50);
7′-cyclopentyl-2′-((2-fluoro-4-(4-methylpiperazin-1-yl)phenyl)amino)-5′,7′-dihydrospiro[pyrrolidine-3,6′-pyrrolo[2,3-d]pyrimidin]-2-one (C51);
7′-cyclopentyl-2′-((2-fluoro-4-(4-isopropylpiperazin-1-yl)phenyl)amino)-5′,7′-dihydrospiro[pyrrolidine-3,6′-pyrrolo[2,3-d]pyrimidin]-2-one (C52);
7′-cyclopentyl-2′-((6-(piperazin-1-yl)pyridin-3-yl)amino)-5′,7′-dihydrospiro[pyrrolidine-3,6′-pyrrolo[2,3-d]pyrimidin]-2-one (C53);
7′-cyclopentyl-2′-((6-(4-methylpiperazin-1-yl)pyridin-3-yl)amino)-5′,7′-dihydrospiro[pyrrolidine-3,6′-pyrrolo[2,3-d]pyrimidin]-2-one (C54);
7′-cyclopentyl-2′-((6-(4-isopropylpiperazin-1-yl)pyridin-3-yl)amino)-5′,7′-dihydrospiro[pyrrolidine-3,6′-pyrrolo[2,3-d]pyrimidin]-2-one (C55);
7′-cyclopentyl-2′-((1′,2′,3′,6′-tetrahydro-[3,4′-bipyridin]-6-yl)amino)-5′,7′-dihydrospiro[pyrrolidine-3,6′-pyrrolo[2,3-d]pyrimidin]-2-one (C56);
7′-cyclopentyl-2′-((1′-isopropyl-1′,2′,3′,6′-tetrahydro-[3,4′-bipyridin]-6-yl)amino)-5′,7′-dihydrospiro[pyrrolidine-3,6′-pyrrolo[2,3-d]pyrimidin]-2-one (C57);
7′-cyclopentyl-2′-((3-fluoro-4-morpholinophenyl)amino)-5′,7′-dihydrospiro[pyrrolidine-3,6′-pyrrolo[2,3-d]pyrimidin]-2-one (C58);
7′-cyclopentyl-2′-((4-(2-(dimethylamino)ethoxy)phenyl)amino)-5′,7′-dihydrospiro[pyrrolidine-3,6′-pyrrolo[2,3-d]pyrimidin]-2-one (C59);
7′-cyclopentyl-2′-((5-(2-(dimethylamino)ethoxy)pyridin-2-yl)amino)-5′,7′-dihydrospiro[pyrrolidine-3,6′-pyrrolo[2,3-d]pyrimidin]-2-one (C60);
7′-cyclopentyl-2′-((6-(2-(dimethylamino)ethoxy)pyridin-3-yl)amino)-5′,7′-dihydrospiro[pyrrolidine-3,6′-pyrrolo[2,3-d]pyrimidin]-2-one (C61);
7′-cyclopentyl-2′-((4-(4-isopropylpiperazin-1-yl)-3-methoxyphenyl)amino)-5′,7′-dihydrospiro[pyrrolidine-3,6′-pyrrolo[2,3-d]pyrimidin]-2-one (C62);
7′-cyclopentyl-2′-((4-(4-isopropylpiperazin-1-yl)-3-methylphenyl)amino)-5′,7′-dihydrospiro[pyrrolidine-3,6′-pyrrolo[2,3-d]pyrimidin]-2-one (C63);
2-((5-(4-cyanopiperazin-1-yl)pyridin-2-yl)amino)-7-cyclopentyl-N,N-dimethyl-7H-pyrrolo[2,3-d]pyrimidine-6-carboxamide (C64);
7′-cyclopentyl-1-methyl-2′-((5-(piperazin-1-yl)pyridin-2-yl)amino)-5′,7′-dihydrospiro[pyrrolidine-3,6′-pyrrolo[2,3-d]pyrimidine]-2,5-dione (C65);
7′-cyclopentyl-1-methyl-2′-((5-(4-methylpiperazin-1-yl)pyridin-2-yl)amino)-5′,7′-dihydrospiro[pyrrolidine-3,6′-pyrrolo[2,3-d]pyrimidine]-2,5-dione (C66);
7′-cyclopentyl-2′-((5-(4-isopropylpiperazin-1-yl)pyridin-2-yl)amino)-1-methyl-5′,7′-dihydrospiro[pyrrolidine-3,6′-pyrrolo[2,3-d]pyrimidine]-2,5-dione (C67);
7′-cyclopentyl-1-methyl-2′-((4-(piperazin-1-yl)phenyl)amino)-5′,7′-dihydrospiro[pyrrolidine-3,6′-pyrrolo[2,3-d]pyrimidine]-2,5-dione (C68);
7′-cyclopentyl-1-methyl-2′-((4-(4-methylpiperazin-1-yl)phenyl)amino)-5′,7′-dihydrospiro[pyrrolidine-3,6′-pyrrolo[2,3-d]pyrimidine]-2,5-dione (C69);
7′-cyclopentyl-2′-((4-(4-isopropylpiperazin-1-yl)phenyl)amino)-1-methyl-5′,7′-dihydrospiro[pyrrolidine-3,6′-pyrrolo[2,3-d]pyrimidine]-2,5-dione (C70);
7′-cyclopentyl-2′-((3-fluoro-4-(piperazin-1-yl)phenyl)amino)-1-methyl-5′,7′-dihydrospiro[pyrrolidine-3,6′-pyrrolo[2,3-d]pyrimidine]-2,5-dione (C72);
7′-cyclopentyl-2′-((3-fluoro-4-(4-methylpiperazin-1-yl)phenyl)amino)-1-methyl-5′,7′-dihydrospiro[pyrrolidine-3,6′-pyrrolo[2,3-d]pyrimidine]-2,5-dione (C72);
7′-cyclopentyl-2′-((3-fluoro-4-(4-isopropylpiperazin-1-yl)phenyl)amino)-1-methyl-5′,7′-dihydrospiro[pyrrolidine-3,6′-pyrrolo[2,3-d]pyrimidine]-2,5-dione (C73);
7′-cyclopentyl-2′-((5-(piperazin-1-yl)pyridin-2-yl)amino)-5′,7′-dihydrospiro[pyrrolidine-3,6′-pyrrolo[2,3-d]pyrimidine]-2,5-dione (C74);
7′-cyclopentyl-2′-((5-(4-methylpiperazin-1-yl)pyridin-2-yl)amino)-5′,7′-dihydrospiro[pyrrolidine-3,6′-pyrrolo[2,3-d]pyrimidine]-2,5-dione (C75);
7′-cyclopentyl-2′-((5-(4-isopropylpiperazin-1-yl)pyridin-2-yl)amino)-5′,7′-dihydrospiro[pyrrolidine-3,6′-pyrrolo[2,3-d]pyrimidine]-2,5-dione (C76);
7′-cyclopentyl-2′-((4-(piperazin-1-yl)phenyl)amino)-5′,7′-dihydrospiro[pyrrolidine-3,6′-pyrrolo[2,3-d]pyrimidine]-2,5-dione (C77);
7′-cyclopentyl-2′-((4-(4-methylpiperazin-1-yl)phenyl)amino)-5′,7′-dihydrospiro[pyrrolidine-3,6′-pyrrolo[2,3-d]pyrimidine]-2,5-dione (C78);
7′-cyclopentyl-2′-((4-(4-isopropylpiperazin-1-yl)phenyl)amino)-5′,7′-dihydrospiro[pyrrolidine-3,6′-pyrrolo[2,3-d]pyrimidine]-2,5-dione (C79);
7′-cyclopentyl-2′-((3-fluoro-4-(piperazin-1-yl)phenyl)amino)-5′,7′-dihydrospiro[pyrrolidine-3,6′-pyrrolo[2,3-d]pyrimidine]-2,5-dione (C80);
7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidine]-2,5-dione (C81);
7′-cyclopentyl-2′-((3-fluoro-4-(4-isopropylpiperazin-1-yl)phenyl)amino)-5′,7′-dihydrospiro[pyrrolidine-3,6′-pyrrolo[2,3-d]pyrimidine]-2,5-dione (C82);
7′-cyclopentyl-2′-((6-(piperazin-1-yl)pyridin-3-yl)amino)-5′,7′-dihydrospiro[pyrrolidine-3,6′-pyrrolo[2,3-d]pyrimidine]-2,5-dione (C83);
7′-cyclopentyl-2′-((6-(4-methylpiperazin-1-yl)pyridin-3-yl)amino)-5′,7′-dihydrospiro[pyrrolidine-3,6′-pyrrolo[2,3-d]pyrimidine]-2,5-dione (C84);
7′-cyclopentyl-2′-((6-(4-isopropylpiperazin-1-yl)pyridin-3-yl)amino)-5′,7′-dihydrospiro[pyrrolidine-3,6′-pyrrolo[2,3-d]pyrimidine]-2,5-dione (C85);
7′-cyclopentyl-2′-((3-fluoro-4-morpholinophenyl)amino)-5′,7′-dihydrospiro[pyrrolidine-3,6′-pyrrolo[2,3-d]pyrimidine]-2,5-dione (C86);
7′-cyclopentyl-2′-((2-fluoro-4-(4-methylpiperazin-1-yl)phenyl)amino)-5′,7′-dihydrospiro[pyrrolidine-3,6′-pyrrolo[2,3-d]pyrimidine]-2,5-dione (C87);
7′-cyclopentyl-2′-((6-(2-(dimethylamino)ethoxy)pyridin-3-yl)amino)-5′,7′-dihydrospiro[pyrrolidine-3,6′-pyrrolo[2,3-d]pyrimidine]-2,5-dione (C88);
7′-cyclopentyl-2′-((4-(2-(dimethylamino)ethoxy)phenyl)amino)-5′,7′-dihydrospiro[pyrrolidine-3,6′-pyrrolo[2,3-d]pyrimidine]-2,5-dione (C89);
7′-cyclopentyl-2′-((6-(2-(pyrrolidin-1-yl)ethoxy)pyridin-3-yl)amino)-5′,7′-dihydrospiro[pyrrolidine-3,6′-pyrrolo[2,3-d]pyrimidine]-2,5-dione (C90);
7′-cyclopentyl-2′-((3-fluoro-4-(2-(pyrrolidin-1-yl)ethoxy)phenyl)amino)-5′,7′-dihydrospiro[pyrrolidine-3,6′-pyrrolo[2,3-d]pyrimidine]-2,5-dione (C91);
7′-cyclopentyl-2′-((5-(2-(dimethylamino)ethoxy)pyridin-2-yl)amino)-5′,7′-dihydrospiro[pyrrolidine-3,6′-pyrrolo[2,3-d]pyrimidine]-2,5-dione (C92);
7′-cyclopentyl-2′-((1′,2′,3′,6′-tetrahydro-[3,4′-bipyridin]-6-yl)amino)-5′,7′-dihydrospiro[pyrrolidine-3,6′-pyrrolo[2,3-d]pyrimidine]-2,5-dione (C93);
7′-cyclopentyl-2′-((1′-isopropyl-1′,2′,3′,6′-tetrahydro-[3,4′-bipyridin]-6-yl)amino)-5′,7′-dihydrospiro[pyrrolidine-3,6′-pyrrolo[2,3-d]pyrimidine]-2,5-dione (C94);
7′-cyclopentyl-2′-((4-(4-morpholinopiperidin-1-yl)phenyl)amino)-5′,7′-dihydrospiro[pyrrolidine-3,6′-pyrrolo[2,3-d]pyrimidin]-2-one (C95);
7′-cyclopentyl-2′-((6-(4-morpholinopiperidin-1-yl)pyridin-3-yl)amino)-5′,7′-dihydrospiro[pyrrolidine-3,6′-pyrrolo[2,3-d]pyrimidin]-2-one (C96);
7′-cyclopentyl-2′-((3-fluoro-4-(4-morpholinopiperidin-1-yl)phenyl)amino)-5′,7′-dihydrospiro[pyrrolidine-3,6′-pyrrolo[2,3-d]pyrimidin]-2-one (C97);
7′-cyclopentyl-2′-((5-(4-methylpiperazin-1-yl)pyridin-2-yl)amino)-6′,7′-dihydrospiro[pyrrolidine-3,5′-pyrrolo[2,3-d]pyrimidin]-2-one (C98);
7′-cyclopentyl-2′-((5-(piperazin-1-yl)pyridin-2-yl)amino)spiro[pyrrolidine-3,5′-pyrrolo[2,3-d]pyrimidine]-2,6′(7′H)-dione (C99);
7′-cyclopentyl-2′-((5-(4-methylpiperazin-1-yl)pyridin-2-yl)amino)spiro[pyrrolidine-3,5′-pyrrolo[2,3-d]pyrimidine]-2,6′(7′H)-dione (C100);
7′-cyclopentyl-2′-((4-(4-methylpiperazin-1-yl)phenyl)amino)spiro[pyrrolidine-3,5′-pyrrolo[2,3-d]pyrimidine]-2,6′(7′H)-dione (C101);
7′-cyclopentyl-2′-((3-fluoro-4-(piperazin-1-yl)phenyl)amino)spiro[pyrrolidine-3,5′-pyrrolo[2,3-d]pyrimidine]-2,6′(7′H)-dione (C102);
7′-cyclopentyl-2′-((3-fluoro-4-(4-methylpiperazin-1-yl)phenyl)amino)spiro[pyrrolidine-3,5′-pyrrolo[2,3-d]pyrimidine]-2,6′(7′H)-dione (C103);
7′-cyclopentyl-2′-((3-fluoro-4-(4-isopropylpiperazin-1-yl)phenyl)amino)spiro[pyrrolidine-3,5′-pyrrolo[2,3-d]pyrimidine]-2,6′(7′H)-dione (C104);
7′-cyclopentyl-2′-((4-((S)-3-(dimethylamino)pyrrolidin-1-yl)-3-fluorophenyl)amino)spiro[pyrrolidine-3,5′-pyrrolo[2,3-d]pyrimidine]-2,6′(7′H)-dione (C105);
7′-cyclopentyl-2′-((3-fluoro-4-(4-morpholinopiperidin-1-yl)phenyl)amino)spiro[pyrrolidine-3,5′-pyrrolo[2,3-d]pyrimidine]-2,6′(7′H)-dione (C106);
7′-cyclopentyl-2′-((3-fluoro-4-(2,6-diazaspiro[3.3]heptan-2-yl)phenyl)amino)-5′,7′-dihydrospiro[pyrrolidine-3,6′-pyrrolo[2,3-d]pyrimidin]-2-one (C107);
7′-cyclopentyl-2′-((4-(2-(dimethylamino)ethoxy)phenyl)amino)-5′,7′-dihydrospiro[pyrrolidine-3,6′-pyrrolo[2,3-d]pyrimidin]-2-one (C108);
7′-cyclohexyl-2′-((4-(2-(dimethylamino)ethoxy)phenyl)amino)-5′,7′-dihydrospiro[pyrrolidine-3,6′-pyrrolo[2,3-d]pyrimidin]-2-one (C109);
7′-cyclopentyl-2′-((6-(2-(dimethylamino)ethoxy)pyridin-3-yl)amino)-5′,7′-dihydrospiro[pyrrolidine-3,6′-pyrrolo[2,3-d]pyrimidin]-2-one (C110);
7′-cyclopentyl-2′-((4-(2-(dimethylamino)ethoxy)-3-fluorophenyl)amino)-5′,7′-dihydrospiro[pyrrolidine-3,6′-pyrrolo[2,3-d]pyrimidin]-2-one (C111);
7′-cyclopentyl-2′-((4-(2-(dimethylamino)ethoxy)-3-methylphenyl)amino)-5′,7′-dihydrospiro[pyrrolidine-3,6′-pyrrolo[2,3-d]pyrimidin]-2-one (C112);
7′-cyclopentyl-2′-((4-(2-(dimethylamino)ethoxy)-3-methoxyphenyl)amino)-5′,7′-dihydrospiro[pyrrolidine-3,6′-pyrrolo[2,3-d]pyrimidin]-2-one (C113);
7′-cyclopentyl-2′-((4-(piperazin-1-yl)phenyl)amino)-5′,7′-dihydrospiro[pyrrolidine-3,6′-pyrrolo[2,3-d]pyrimidin]-2-one (C114);
7′-cyclopentyl-2′-((4-(4-methylpiperazin-1-yl)phenyl)amino)-5′,7′-dihydrospiro[pyrrolidine-3,6′-pyrrolo[2,3-d]pyrimidin]-2-one (C115);
7′-cyclopentyl-2′-((4-(4-isopropylpiperazin-1-yl)phenyl)amino)-5′,7′-dihydrospiro[pyrrolidine-3,6′-pyrrolo[2,3-d]pyrimidin]-2-one (C116);
7′-cyclopentyl-2′-((3-fluoro-4-(piperazin-1-yl)phenyl)amino)-5′,7′-dihydrospiro[pyrrolidine-3,6′-pyrrolo[2,3-d]pyrimidin]-2-one (C117);
7′-cyclopentyl-2′-((3-fluoro-4-(4-isopropylpiperazin-1-yl)phenyl)amino)-5′,7′-dihydrospiro[pyrrolidine-3,6′-pyrrolo[2,3-d]pyrimidin]-2-one (C118);
7′-cyclopentyl-2′-((3-methyl-4-(piperazin-1-yl)phenyl)amino)-5′,7′-dihydrospiro[pyrrolidine-3,6′-pyrrolo[2,3-d]pyrimidin]-2-one (C119);
7′-cyclopentyl-2′-((3-methyl-4-(4-methylpiperazin-1-yl)phenyl)amino)-5′,7′-dihydrospiro[pyrrolidine-3,6′-pyrrolo[2,3-d]pyrimidin]-2-one (C120);
7′-cyclopentyl-2′-((4-(4-isopropylpiperazin-1-yl)-3-methylphenyl)amino)-5′,7′-dihydrospiro[pyrrolidine-3,6′-pyrrolo[2,3-d]pyrimidin]-2-one (C121);
7′-cyclopentyl-2′-((3-methoxy-4-(piperazin-1-yl)phenyl)amino)-5′,7′-dihydrospiro[pyrrolidine-3,6′-pyrrolo[2,3-d]pyrimidin]-2-one (C122);
7′-cyclopentyl-2′-((3-methoxy-4-(4-methylpiperazin-1-yl)phenyl)amino)-5′,7′-dihydrospiro[pyrrolidine-3,6′-pyrrolo[2,3-d]pyrimidin]-2-one (C123);
7′-cyclopentyl-2′-((4-(4-isopropylpiperazin-1-yl)-3-methoxyphenyl)amino)-5′,7′-dihydrospiro[pyrrolidine-3,6′-pyrrolo[2,3-d]pyrimidin]-2-one (C124);
7′-cyclopentyl-2′-((3-fluoro-4-(2,6-diazaspiro[3.3]heptan-2-yl)phenyl)amino)-5′,7′-dihydrospiro[pyrrolidine-3,6′-pyrrolo[2,3-d]pyrimidin]-2-one (C125);
2′-((4-(2,6-diazaspiro[3.3]heptan-2-yl)phenyl)amino)-7′-cyclopentyl-5′,7′-dihydrospiro[pyrrolidine-3,6′-pyrrolo[2,3-d]pyrimidin]-2-one (C126);
2′-((6-(2,6-diazaspiro[3.3]heptan-2-yl)pyridin-3-yl)amino)-7′-cyclopentyl-5′,7′-dihydrospiro[pyrrolidine-3,6′-pyrrolo[2,3-d]pyrimidin]-2-one (C127);
7′-cyclopentyl-2′-((6-(piperazin-1-yl)pyridin-3-yl)amino)-5′,7′-dihydrospiro[pyrrolidine-3,6′-pyrrolo[2,3-d]pyrimidin]-2-one (C128);
7′-cyclopentyl-2′-((6-(4-methylpiperazin-1-yl)pyridin-3-yl)amino)-5′,7′-dihydrospiro[pyrrolidine-3,6′-pyrrolo[2,3-d]pyrimidin]-2-one (C129);
7′-cyclopentyl-2′-((6-(4-isopropylpiperazin-1-yl)pyridin-3-yl)amino)-5′,7′-dihydrospiro[pyrrolidine-3,6′-pyrrolo[2,3-d]pyrimidin]-2-one (C130);
7′-cyclopentyl-2′-((4-(4-morpholinopiperidin-1-yl)phenyl)amino)-5′,7′-dihydrospiro[pyrrolidine-3,6′-pyrrolo[2,3-d]pyrimidin]-2-one (C131);
7′-cyclopentyl-2′-((3-fluoro-4-(4-morpholinopiperidin-1-yl)phenyl)amino)-5′,7′-dihydrospiro[pyrrolidine-3,6′-pyrrolo[2,3-d]pyrimidin]-2-one (C132);
7′-cyclopentyl-2′-((6-(4-morpholinopiperidin-1-yl)pyridin-3-yl)amino)-5′,7′-dihydrospiro[pyrrolidine-3,6′-pyrrolo[2,3-d]pyrimidin]-2-one (C133);
7′-cyclopentyl-2′-((4-(4-isopropylpiperazin-1-yl)phenyl)amino)-5′,7′-dihydrospiro[pyrrolidine-3,6′-pyrrolo[2,3-d]pyrimidine]-2,5-dione (C134);
7′-cyclopentyl-2′-((4-(4-isopropylpiperazin-1-yl)phenyl)amino)-5′,7′-dihydrospiro[pyrrolidine-3,6′-pyrrolo[2,3-d]pyrimidine]-2,5-dione (C135);
7′-cyclopentyl-2′-((4-(4-methylpiperazin-1-yl)phenyl)amino)-5′,7′-dihydrospiro[pyrrolidine-3,6′-pyrrolo[2,3-d]pyrimidine]-2,5-dione (C136);
7′-cyclopentyl-2′-((4-(4-methylpiperazin-1-yl)phenyl)amino)-5′,7′-dihydrospiro[pyrrolidine-3,6′-pyrrolo[2,3-d]pyrimidine]-2,5-dione (C137);
7′-cyclopentyl-2′-((4-(piperazin-1-yl)phenyl)amino)-5′,7′-dihydrospiro[pyrrolidine-3,6′-pyrrolo[2,3-d]pyrimidine]-2,5-dione (C138);
7′-cyclopentyl-2′-((4-(piperazin-1-yl)phenyl)amino)-5′,7′-dihydrospiro[pyrrolidine-3,6′-pyrrolo[2,3-d]pyrimidine]-2,5-dione (C139);
7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidine]-2,5-dione (C140);
7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidine]-2,5-dione (C141);
7′-cyclohexyl-2′-((4-(piperazin-1-yl)phenyl)amino)-5′,7′-dihydrospiro[pyrrolidine-3,6′-pyrrolo[2,3-d]pyrimidin]-2-one (C142);
7′-cyclohexyl-2′-((4-(piperazin-1-yl)phenyl)amino)-5′,7′-dihydrospiro[pyrrolidine-3,6′-pyrrolo[2,3-d]pyrimidin]-2-one (C143);
7′-cyclohexyl-2′-((4-(4-methylpiperazin-1-yl)phenyl)amino)-5′,7′-dihydrospiro[pyrrolidine-3,6′-pyrrolo[2,3-d]pyrimidin]-2-one (C144);
7′-cyclohexyl-2′-((4-(4-methylpiperazin-1-yl)phenyl)amino)-5′,7′-dihydrospiro[pyrrolidine-3,6′-pyrrolo[2,3-d]pyrimidin]-2-one (C145);
7′-cyclohexyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one (C146);
7′-cyclohexyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one (C147);
7′-cyclopentyl-2′-((4-(6-methyl-2,6-diazaspiro[3.3]heptan-2-yl)phenyl)amino)-5′,7′-dihydrospiro[pyrrolidine-3,6′-pyrrolo[2,3-d]pyrimidine]-2,5-dione (C148);
7′-cyclopentyl-2′-((3-fluoro-4-(6-methyl-2,6-diazaspiro[3.3]heptan-2-yl)phenyl)amino)-5′,7′-dihydrospiro[pyrrolidine-3,6′-pyrrolo[2,3-d]pyrimidine]-2,5-dione (C149);
7′-cyclopentyl-2′-((4-(6-isopropyl-2,6-diazaspiro[3.3]heptan-2-yl)phenyl)amino)-5′,7′-dihydrospiro[pyrrolidine-3,6′-pyrrolo[2,3-d]pyrimidine]-2,5-dione (C150);
7′-cyclopentyl-2′-((3-fluoro-4-(6-isopropyl-2,6-diazaspiro[3.3]heptan-2-yl)phenyl)amino)-5′,7′-dihydrospiro[pyrrolidine-3,6′-pyrrolo[2,3-d]pyrimidine]-2,5-dione (C151);
7′-cyclopentyl-2′-((1-(methylsulfonyl)piperidin-4-yl)amino)-5′,7′-dihydrospiro[pyrrolidine-3,6′-pyrrolo[2,3-d]pyrimidine]-2,5-dione (C152);
2′-(bicyclo[2.2.1]heptan-2-ylamino)-7′-cyclopentyl-5′,7′-dihydrospiro[pyrrolidine-3,6′-pyrrolo[2,3-d]pyrimidine]-2,5-dione (C153);
7′-cyclopentyl-2′-(((1 r,4r)-4-((tetrahydro-2H-pyran-4-yl)amino)cyclohexyl)amino)-5′,7′-dihydrospiro[pyrrolidine-3,6′-pyrrolo[2,3-d]pyrimidine]-2,5-dione (C154);
7′-cyclopentyl-2′-((3-fluoro-4-isopropylphenyl)amino)-5′,7′-dihydrospiro[pyrrolidine-3,6′-pyrrolo[2,3-d]pyrimidine]-2,5-dione (C155);
7′-cyclopentyl-2′-((4-(6-methyl-2,6-diazaspiro[3.3]heptan-2-yl)phenyl)amino)-5′,7′-dihydrospiro[pyrrolidine-3,6′-pyrrolo[2,3-d]pyrimidin]-2-one (C156);
7′-cyclopentyl-2′-((3-fluoro-4-(6-methyl-2,6-diazaspiro[3.3]heptan-2-yl)phenyl)amino)-5′,7′-dihydrospiro[pyrrolidine-3,6′-pyrrolo[2,3-d]pyrimidin]-2-one (C157);
7′-cyclopentyl-2′-((4-(6-isopropyl-2,6-diazaspiro[3.3]heptan-2-yl)phenyl)amino)-5′,7′-dihydrospiro[pyrrolidine-3,6′-pyrrolo[2,3-d]pyrimidin]-2-one (C158);
7′-cyclopentyl-2′-((3-fluoro-4-(6-isopropyl-2,6-diazaspiro[3.3]heptan-2-yl)phenyl)amino)-5′,7′-dihydrospiro[pyrrolidine-3,6′-pyrrolo[2,3-d]pyrimidin]-2-one (C159);
7′-cyclopentyl-2′-((4-(methylsulfonyl)phenyl)amino)-5′,7′-dihydrospiro[pyrrolidine-3,6′-pyrrolo[2,3-d]pyrimidin]-2-one (C160);
7′-cyclopentyl-2′-((1-isopropylpiperidin-4-yl)amino)-5′,7′-dihydrospiro[pyrrolidine-3,6′-pyrrolo[2,3-d]pyrimidin]-2-one (C161);
7′-cyclopentyl-2′-(((1 r,4r)-4-((tetrahydro-2H-pyran-4-yl)amino)cyclohexyl)amino)-5′,7′-dihydrospiro[pyrrolidine-3,6′-pyrrolo[2,3-d]pyrimidin]-2-one (C162);
7′-cyclopentyl-2′-(((1 S,2S)-2-hydroxy-2-methylcyclopentyl)amino)-5′,7′-dihydrospiro[pyrrolidine-3,6′-pyrrolo[2,3-d]pyrimidin]-2-one (C163);
7′-cyclopentyl-2′-((4-(1-methylpiperidin-4-yl)phenyl)amino)-5′,7′-dihydrospiro[pyrrolidine-3,6′-pyrrolo[2,3-d]pyrimidin]-2-one (C164);
7′-cyclopentyl-2′-((4-(2-morpholinoethoxy)phenyl)amino)-5′,7′-dihydrospiro[pyrrolidine-3,6′-pyrrolo[2,3-d]pyrimidin]-2-one (C165);
7′-cyclopentyl-2′-((3-fluoro-4-morpholinophenyl)amino)-5′,7′-dihydrospiro[pyrrolidine-3,6′-pyrrolo[2,3-d]pyrimidin]-2-one (C166);
7′-cyclopentyl-2′-((6-(2-(dimethylamino)ethoxy)pyridin-3-yl)amino)-5′,7′-dihydrospiro[pyrrolidine-3,6′-pyrrolo[2,3-d]pyrimidin]-2-one (C167);
7′-cyclopentyl-2′-((4-(1-methyl-1H-pyrazol-4-yl)phenyl)amino)-5′,7′-dihydrospiro[pyrrolidine-3,6′-pyrrolo[2,3-d]pyrimidin]-2-one (C168);
7′-cyclopentyl-2′-((4-(1-isopropyl-1H-pyrazol-4-yl)phenyl)amino)-5′,7′-dihydrospiro[pyrrolidine-3,6′-pyrrolo[2,3-d]pyrimidin]-2-one (C169);
7′-cyclopentyl-2′-((6-(2-(pyrrolidin-1-yl)ethoxy)pyridin-3-yl)amino)-5′,7′-dihydrospiro[pyrrolidine-3,6′-pyrrolo[2,3-d]pyrimidin]-2-one (C170);
7′-cyclopentyl-2′-((6-(2-(piperidin-1-yl)ethoxy)pyridin-3-yl)amino)-5′,7′-dihydrospiro[pyrrolidine-3,6′-pyrrolo[2,3-d]pyrimidin]-2-one (C171);
7′-cyclopentyl-2′-((4-((S)-3-(dimethylamino)pyrrolidin-1-yl)phenyl)amino)-5′,7′-dihydrospiro[pyrrolidine-3,6′-pyrrolo[2,3-d]pyrimidin]-2-one (C172);
7′-cyclopentyl-2′-((4-((S)-3-(dimethylamino)pyrrolidin-1-yl)-3-fluorophenyl)amino)-5′,7′-dihydrospiro[pyrrolidine-3,6′-pyrrolo[2,3-d]pyrimidin]-2-one (C173);
7′-cyclopentyl-2′-((6-((S)-3-(dimethylamino)pyrrolidin-1-yl)pyridin-3-yl)amino)-5′,7′-dihydrospiro[pyrrolidine-3,6′-pyrrolo[2,3-d]pyrimidin]-2-one (C174);
7′-cyclopentyl-2′-((4-((R)-3-(dimethylamino)pyrrolidin-1-yl)phenyl)amino)-5′,7′-dihydrospiro[pyrrolidine-3,6′-pyrrolo[2,3-d]pyrimidin]-2-one (C175);
7′-cyclopentyl-2′-((4-((R)-3-(dimethylamino)pyrrolidin-1-yl)-3-fluorophenyl)amino)-5′,7′-dihydrospiro[pyrrolidine-3,6′-pyrrolo[2,3-d]pyrimidin]-2-one (C176);
7′-cyclopentyl-2′-((6-((R)-3-(dimethylamino)pyrrolidin-1-yl)pyridin-3-yl)amino)-5′,7′-dihydrospiro[pyrrolidine-3,6′-pyrrolo[2,3-d]pyrimidin]-2-one (C177);
7′-cyclopentyl-2′-((4-((S)-3-(dimethylamino)pyrrolidine-1-carbonyl)phenyl)amino)-5′,7′-dihydrospiro[pyrrolidine-3,6′-pyrrolo[2,3-d]pyrimidin]-2-one (C178);
7′-cyclopentyl-2′-((4-((S)-3-(dimethylamino)pyrrolidine-1-carbonyl)-3-fluorophenyl)amino)-5′,7′-dihydrospiro[pyrrolidine-3,6′-pyrrolo[2,3-d]pyrimidin]-2-one (C179);
7′-cyclopentyl-2′-((6-((S)-3-(dimethylamino)pyrrolidine-1-carbonyl)pyridin-3-yl)amino)-5′,7′-dihydrospiro[pyrrolidine-3,6′-pyrrolo[2,3-d]pyrimidin]-2-one (C180);
7′-cyclopentyl-2′-((4-((R)-3-(dimethylamino)pyrrolidine-1-carbonyl)phenyl)amino)-5′,7′-dihydrospiro[pyrrolidine-3,6′-pyrrolo[2,3-d]pyrimidin]-2-one (C181);
7′-cyclopentyl-2′-((4-((R)-3-(dimethylamino)pyrrolidine-1-carbonyl)-3-fluorophenyl)amino)-5′,7′-dihydrospiro[pyrrolidine-3,6′-pyrrolo[2,3-d]pyrimidin]-2-one (C182);
7′-cyclopentyl-2′-((6-((R)-3-(dimethylamino)pyrrolidine-1-carbonyl)pyridin-3-yl)amino)-5′,7′-dihydrospiro[pyrrolidine-3,6′-pyrrolo[2,3-d]pyrimidin]-2-one (C183);
7′-cyclopentyl-2′-((4-isopropylphenyl)amino)-5′,7′-dihydrospiro[pyrrolidine-3,6′-pyrrolo[2,3-d]pyrimidin]-2-one (C184);
7′-cyclopentyl-2′-((3-fluoro-4-isopropylphenyl)amino)-5′,7′-dihydrospiro[pyrrolidine-3,6′-pyrrolo[2,3-d]pyrimidin]-2-one (C185);
2′-((4-acetyl-3-fluorophenyl)amino)-7′-cyclopentyl-5′,7′-dihydrospiro[pyrrolidine-3,6′-pyrrolo[2,3-d]pyrimidin]-2-one (C186);
2′-((4-(tert-butyl)-3-fluorophenyl)amino)-7′-cyclopentyl-5′,7′-dihydrospiro[pyrrolidine-3,6′-pyrrolo[2,3-d]pyrimidin]-2-one (C187);
7′-cyclopentyl-2′-((4-hydroxy-4-methylcyclohexyl)amino)-5′,7′-dihydrospiro[pyrrolidine-3,6′-pyrrolo[2,3-d]pyrimidin]-2-one (C188);
7′-cyclopentyl-2′-(((1s,4s)-4-hydroxy-4-methylcyclohexyl)amino)-5′,7′-dihydrospiro[pyrrolidine-3,6′-pyrrolo[2,3-d]pyrimidin]-2-one (C189);
7′-cyclopentyl-2′-(((1 r,4r)-4-hydroxy-4-methylcyclohexyl)amino)-5′,7′-dihydrospiro[pyrrolidine-3,6′-pyrrolo[2,3-d]pyrimidin]-2-one (C190);
7′-cyclopentyl-2′-((5-(2-(dimethylamino)ethoxy)pyridin-2-yl)amino)-5′,7′-dihydrospiro[pyrrolidine-3,6′-pyrrolo[2,3-d]pyrimidin]-2-one (C191);
7′-cyclopentyl-2′-((5-(2-(pyrrolidin-1-yl)ethoxy)pyridin-2-yl)amino)-5′,7′-dihydrospiro[pyrrolidine-3,6′-pyrrolo[2,3-d]pyrimidin]-2-one (C192);
7′-cyclopentyl-2′-((5-(2-(piperidin-1-yl)ethoxy)pyridin-2-yl)amino)-5′,7′-dihydrospiro[pyrrolidine-3,6′-pyrrolo[2,3-d]pyrimidin]-2-one (C193);
(S)-7′-cyclopentyl-2′-(((1S,2S,4R)-1,7,7-trimethylbicyclo[2.2.1]heptan-2-yl)amino)-5′,7′-dihydrospiro[pyrrolidine-3,6′-pyrrolo[2,3-d]pyrimidin]-2-one (C194);
(S)-7′-cyclopentyl-2′-(((1R,2S,4R)-1,7,7-trimethylbicyclo[2.2.1]heptan-2-yl)amino)-5′,7′-dihydrospiro[pyrrolidine-3,6′-pyrrolo[2,3-d]pyrimidin]-2-one (C195);
(S)-7′-cyclopentyl-2′-(((1S,2S,3S,5R)-2,6,6-trimethylbicyclo[3.1.1]heptan-3-yl)amino)-5′,7′-dihydrospiro[pyrrolidine-3,6′-pyrrolo[2,3-d]pyrimidin]-2-one (C196);
7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one (C197);
7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidine]-2,5-dione (C198);
7′-cyclohexyl-2′-((4-(4-methylpiperazin-1-yl)phenyl)amino)-5′,7′-dihydrospiro[pyrrolidine-3,6′-pyrrolo[2,3-d]pyrimidin]-2-one (C199);
7′-cyclohexyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one (C200);
7′-cyclohexyl-2′-((6-(4-methylpiperazin-1-yl)pyridin-3-yl)amino)-5′,7′-dihydrospiro[pyrrolidine-3,6′-pyrrolo[2,3-d]pyrimidin]-2-one (C201);
7′-cyclohexyl-2′-((5-(4-methylpiperazin-1-yl)pyridin-2-yl)amino)-5′,7′-dihydrospiro[pyrrolidine-3,6′-pyrrolo[2,3-d]pyrimidin]-2-one (C202);
7′-cyclopentyl-2′-((3-fluoro-4-(piperazin-1-yl)phenyl)amino)-5′,7′-dihydrospiro[pyrrolidine-3,6′-pyrrolo[2,3-d]pyrimidin]-2-one (C203);
7′-cyclopentyl-2′-((3-fluoro-4-(piperazin-1-yl)phenyl)amino)-5′,7′-dihydrospiro[pyrrolidine-3,6′-pyrrolo[2,3-d]pyrimidin]-2-one (C204);
7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one (C205);
7′-cyclopentyl-2′-((3-fluoro-4-(4-methylpiperazin-1-yl)phenyl)amino)-5′,7′-dihydrospiro[pyrrolidine-3,6′-pyrrolo[2,3-d]pyrimidin]-2-one (C206);
7′-cyclopentyl-2′-((4-(3,5-dimethylpiperazin-1-yl)phenyl)amino)-5′,7′-dihydrospiro[pyrrolidine-3,6′-pyrrolo[2,3-d]pyrimidin]-2-one (C207);
7′-cyclopentyl-2′-((4-(3,5-dimethylpiperazin-1-yl)phenyl)amino)-5′,7′-dihydrospiro[pyrrolidine-3,6′-pyrrolo[2,3-d]pyrimidine]-2,5-dione (C208);
7′-cyclopentyl-2′-((4-isopropylphenyl)amino)-5′,7′-dihydrospiro[pyrrolidine-3,6′-pyrrolo[2,3-d]pyrimidin]-2-one (C209);
7′-cyclopentyl-2′-((5-(2-(dimethylamino)ethoxy)pyridin-2-yl)amino)-5′,7′-dihydrospiro[pyrrolidine-3,6′-pyrrolo[2,3-d]pyrimidin]-2-one (C210);
tert-butyl(S)-7′-cyclopentyl-2′-(methylthio)-2-oxo-5′,7′-dihydrospiro[pyrrolidine-3,6′-pyrrolo[2,3-d]pyrimidine]-1-carboxylate (C211);
tert-butyl(S)-7′-cyclohexyl-2′-(methylthio)-2-oxo-5′,7′-dihydrospiro[pyrrolidine-3,6′-pyrrolo[2,3-d]pyrimidine]-1-carboxylate (C212); or
pharmaceutically acceptable salts thereof.
